(19) United States
(12) Patent Application Publication
(10) Pub. No.: US 2011/0166121 A1
(43) Pub. Date: Jul. 7, 2011

(54) FUSED HETEROCYCLIC COMPOUND

(75) Inventors: Chang Seok Lee, Daejeon (KR); Tae Hee Lee, Daejeon (KR); Sook Kyung Yoon, Daejeon (KR); Jeung Soon Choi, Daejeon (KR); Yong Jin Jang, Daejeon (KR); Sung Wook Kim, Daejeon (KR); Hye Kyung Chang, Daejeon (KR); Mi Jeong Park, Daejeon (KR); Tae Hun Kim, Daejeon (KR); Young Ha Ahn, Daejeon (KR); Hee Dong Park, Daejeon (KR); Hyun Jung Park, Daejeon (KR); Dong Chul Lim, Daejeon (KR); Joo Youn Lee, Daejeon (KR); Sung Hack Lee, Daejeon (KR); Wan Su Park, Daejeon (KR); Yeong Soo Oh, Daejeon (KR)

(73) Assignee: LG LIFE SCIENCES LTD., Seoul (KR)

(21) Appl. No.: 13/059,832

(22) PCT Filed: Sep. 8, 2009
(86) PCT No.: PCT/KR2009/005073

§ 371 Date: Mar. 23, 2011

(30) Foreign Application Priority Data
Sep. 8, 2008 (KR) 10-2008-0088483

(51) Int. Cl. A61K 31/397; C07D 487/04; A61K 31/4985; A61K 31/496; A61K 31/519; A61P 9/00; A61P 7/00

(52) U.S. Cl. 514/210.18; 544/278; 514/249; 514/252.16; 514/260.1

(57) ABSTRACT

The present invention relates to a fused heterocyclic compound having the Formula 1, which is useful as a platelet aggregation inhibitor, a method for preparing the same, and a pharmaceutical composition for inhibiting platelet aggregation comprising the same.

### TECHNICAL FIELD

The present invention relates to a fused heterocyclic compound having the following Formula 1 or a pharmaceutically acceptable salt thereof, which is useful as a platelet aggregation inhibitor.

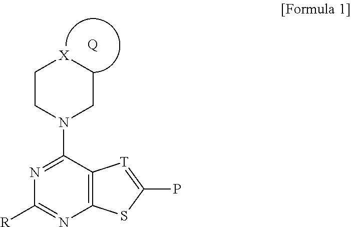

wherein P, Q, R, T and X are as defined herein.

The present invention also relates to a method for preparing a fused heterocyclic compound having Formula 1 or a pharmaceutically acceptable salt thereof.

The present invention also relates to a pharmaceutical composition for inhibiting platelet aggregation comprising a fused heterocyclic compound having Formula 1 or a pharmaceutically acceptable salt thereof as active ingredient.

### BACKGROUND ART

For a long time, platelet has been regarded as an essential element for hemostasis. Hemostasis is a body protection process that stops bleeding from impaired blood vessels. However, an abnormal hemostasis in blood vessels might generate blood clots. Platelet is an important cause of the generation and growth of blood clots in blood vessels. In case platelet is activated by an irregular blood flow condition in blood vessels with disease or by release of a mediator from impaired blood vessel endothelial cells or other circulation cells, it might increase the size of blood clots so that blood clots would close arterial blood vessels at the impaired region of blood vessels. Vein blood clots can be partially and easily separated as an embolus, which migrates through a circulatory organ and may cause occlusion of other vessels. Arterial blood clots cause serious disorders by local occlusion, whereas vein blood clots generally cause long-distance occlusion or occlusion by embolus. These conditions may result in pathological phenomena, such as vascular ischaemic events, acute coronary syndrome without ST-segment elevation (NSTEMI), ST elevation MI (STEMI), peripheral arterial disease, acute coronary syndrome (ACS), phlebothrombosis, thrombophlebitis, arterial embolism, coronary and cerebral arterial thrombosis, unstable angina, myocardial infarction, stroke, cerebral embolism, renal embolism or pulmonary embolism.

Hematogenous reconstruction, such as percutaneous coronary intervention (with or without stent), coronary artery bypass graft surgery (CABG), percutaneous transluminal coronary angioplasty (PTCA) or stent insertion, has rapidly been propagated and used for the treatment of coronary arterial stenosis such as angina or myocardial infarction, or aortic stenosis. However, these treatment methods may harm blood vessel tissues including endothelial cells, resulting in acute coronary occlusion and further restenosis that occurs in a chronic state. It is known that platelet plays an important role in various thrombotic occlusions after hematogenous reconstruction. Thus, there is a need for developing a platelet inhibitor that exhibits high efficacy and stability.

A variety of platelet-growth inhibitors, such as aspirin, cilostazol, prostaglandin I₂, prostaglandin E₁, ticlopidin, dipyridamole, thienopyridine, disintegrin and the like, have been used for the prevention or treatment of circulatory organ diseases. Among them, aspirin and dipyridamole have been used as preventive antithrombotic agents and other agents have been used for clinical purposes. It has been known that agents such as aspirin exhibit only a limited effect, whereas strong agents such as disintegrin, thienopyridine and ticlopidin have substantial side effects.

Recently, a GPIIb/IIIa antagonist has been developed that inhibits the final stage of platelet aggregation and has a strong platelet-aggregation inhibitory activity (U.S. Pat. No. 6,037,343, U.S. Pat. No. 6,040,317). However, its use was limited to only intravenous drip injection at the acute phase of thrombosis.

It has been reported that adenosine 5′-diphosphate (ADP) acts as an important mediator for platelet activation and aggregation (Curr. Opin. Drug Discovery & Development 2001, 4(5) 665-670). ADP induces platelet aggregation, morphological modification, secretion, Ca²⁺ influx and intracellular migration, and adenylyl cyclase inhibition. At least three types of P2 receptor exist in human platelet. P2X1 receptor is involved in rapid calcium influx and can be activated by ATP and ADP. However, its direct role in the platelet-aggregation process is not clear. P2Y1 receptor is involved in calcium migration, morphological modification and initiation of aggregation. P2Y12 receptor is involved in adenylyl cyalase inhibition, a complete induction in response to ADP and stability of aggregation. Both P2Y1 and P2Y12 receptors should be activated for platelet aggregation by ADP. An antagonist that can independently or doubly inhibit these receptors' function will be useful as an anti-platelet formulation.

A variety of platelet receptor antagonists have been reported to exhibit platelet-aggregation inhibition and antithrombotic effects. The most effective known antagonists include thienopyridine, ticlopidin, clopidogrel and CS-747, which have been clinically used as antithrombotic agents (Anesthesia 2003, 58, 28-35; The Lancet 1996, 348, 1329-39; Drugs of the Future 2001, 26(9), 835-840). It has been reported that these drugs irreversibly inhibit ADP-receptor, P2Y12, via activated metabolites.

Adenosine 5′-triphosphate (ATP) derivative, AR-C69931MX (Cangrelor), which is an endogenous antagonist, is a selective P2Y12 antagonist which reversibly inhibits ADP-associated platelet aggregation and is under phase II clinical trial (Curr. Opin. Invest. Drug, 2001, 2(2), 250-255).

In addition, triazolo[4,5-d]pyrimidine derivative (WO 00/034283) and quinoline and piperazine derivative (WO 02/098856 and WO 03/022214) have been reported as compounds having P2Y12 inhibitory activity.

Examples of thienopyrimidine-based P2Y12 receptor antagonist include WO 03/022214 by Pfizer. The compounds disclosed in this document have a thienopyrimidine ring structure, wherein non-fused piperazine ring is substituted.

At the present, the use of known anti-platelet agents and anticoagulants is restrictive because of low efficacy and significant problems with hemorrhaging. Thus, there is an increased need for P2Y12 receptor antagonist having high efficacy and suitable for oral administration.

### DISCLOSURE OF INVENTION

### Technical Problem

The purpose of the present invention is to provide compounds having such valuable pharmaceutical characteristics.

The present inventors newly designed and synthesized compounds having novel chemical structures as inhibitors which are more effective and highly selective to platelet aggregation, and then measured their binding and inhibiting ability to platelet activated by ADP. As a result, the present inventors found that the compounds having the following Formula 1 met with the above purpose, and completed the present invention.

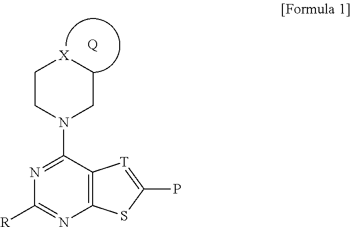

wherein P, Q, R, T and X are as defined below.

Accordingly, the present invention seeks to provide novel, fused heterocyclic compounds having the above Formula 1 or a pharmaceutically acceptable salt thereof which are useful as a platelet aggregation inhibitor.

Furthermore, the present invention seeks to provide a pharmaceutical composition comprising a compound of the above Formula 1 or a pharmaceutically acceptable salt thereof as active ingredient together with pharmaceutically acceptable carrier, for inhibiting platelet aggregation, more concretely, for anti-inflammation or apoptosis inhibition.

### Solution to Problem

The present invention relates to a novel compound having the following Formula 1 or pharmaceutically acceptable salt thereof:

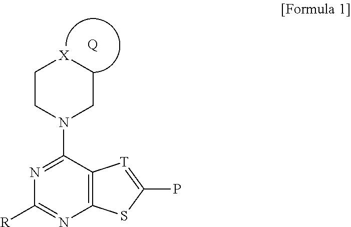

wherein

X represents N or C,

T represents N or C,

the ring Q represents a 3- to 7-membered aromatic ring which comprises 0 to 3 nitrogen atoms as ring members and is optionally benzo-fused, wherein the aromatic ring may be optionally substituted with oxo; alkyl; halogenoalkyl; hydroxyalkyl; alkoxy; aryl; or 3- to 7-membered heteroaryl comprising 1 to 3 heteroatoms selected from nitrogen atom, oxygen atom and sulfur atom,

P represents alkyl being optionally substituted with halogen, and

R represents a group selected from the following groups:

(i)-alkyl-R¹

wherein R¹ is selected from hydroxy; carboxy; carbamoyl; thiocarbamoyl; alkoxycarbonyl; aryloxy being optionally substituted with carboxy or alkoxycarbonyl; arylcarbonyloxy; 3- to 7-membered heteroaryl comprising 1 to 3 heteroatoms selected from nitrogen atom, oxygen atom and sulfur atom, and being optionally substituted with carboxy or alkoxycarbonyl; and 3- to 7-membered heterocycle comprising 1 to 3 heteroatoms selected from nitrogen atom, oxygen atom and sulfur atom, and being optionally substituted with hydroxy.

(ii) —NR²R³

wherein each of R² and R³ is independently selected from hydrogen; alkyl being optionally substituted with amino (said amino is optionally substituted with formyl, alkylcarbonyl, alkoxycarbonyl or carbamoyl), cyano, carbamoyl, hydroxy, carboxy, hydroxyaryl, alkoxy, alkoxycarbonyl, hydroxyalkoxy, 3- to 7-membered heterocycle comprising 1 to 3 heteroatoms selected from nitrogen atom, oxygen atom and sulfur atom (said heterocycle is optionally substituted with oxo, aralkyl, alkylcarbonyl or alkoxycarbonyl), or 3- to 7-membered heteroaryl comprising 1 to 3 heteroatoms selected from nitrogen atom, oxygen atom and sulfur atom; alkylcarbonyl; formyl; alkoxycarbonyl; carbamoyl; cycloalkyl being optionally substituted with hydroxy or hydroxyalkoxy; 3- to 7-membered heterocycle comprising 1 to 3 heteroatoms selected from nitrogen atom, oxygen atom and sulfur atom (said heterocycle is optionally substituted with alkylcarbonyl); aryl; aralkyl; 3- to 7-membered heteroaryl comprising 1 to 3 heteroatoms selected from nitrogen atom, oxygen atom and sulfur atom, and being optionally substituted with carboxy or alkoxycarbonyl.

(iii) —O—R⁴

wherein R⁴ is selected from the following groups:

(a) hydrogen,

(b) alkyl being optionally substituted with hydroxy; alkoxy; amino(said amino is optionally substituted with alkyl, hydroxyalkyl, alkylcarbonyloxyalkyl, formyl, alkylcarbonyl, carbamoyl, alkylaminocarbonyl or alkoxycarbonyl); halogen; cyano; carbamoyl; hydrazidocarbonyl; carboxy; oxo; alkylcarbonyloxyalkoxy; aryl being optionally substituted with halogen; 3- to 7-membered heteroaryl comprising 1 to 3 heteroatoms selected from nitrogen atom, oxygen atom and sulfur atom, and being optionally substituted with carboxyalkyl or alkoxycarbonylalkyl; 3- to 7-membered heterocycle comprising 1 to 3 heteroatoms selected from nitrogen atom, oxygen atom and sulfur atom, and being optionally substituted with oxo, alkylcarbonyl or alkoxycarbonyl; 3- to 7-membered heteroarylcarbonylamino comprising 1 to 3 heteroatoms selected from nitrogen atom, oxygen atom and sulfur atom in the heteroaryl, and being optionally substituted with halogen; 3- to 7-membered heterocyclylcarbonyl comprising 1 to 3 heteroatoms selected from nitrogen atom, oxygen atom and sulfur atom in the heterocycle; 3- to 7-membered heterocyclylcarbonylamino comprising 1 to 3 heteroatoms selected from nitrogen atom, oxygen atom and sulfur atom in the heterocycle; aryloxycarbonylamino being optionally substituted with halogen; cycloalkylaminocarbonyl; or arylcarbonylamino being optionally substituted with halogen,

(c) cycloalkyl being optionally benzo-fused,

(d) alkylaminoalkyl being optionally substituted with alkoxycarbonyl or carboxy,

(e) cycloalkylcarbonylaminoalkyl,

(f) cycloalkylsulfonylaminoalkyl,

(g) alkylcarbonylaminoalkyl being optionally substituted with hydroxy, halogen, amino, alkoxy, alkylsulfonyl or aminosulfonyl,

(h) alkylsulfonylaminoalkyl being optionally substituted with halogen,

(i) aryl being optionally substituted with cyano; formyl; carboxy; alkoxycarbonyl; hydroxyalkyl; carboxyalkyl; alkoxycarbonylalkyl; carboxyalkoxy; alkoxycarbonylalkoxy; or 3- to 7-membered heterocycle comprising 1 to 3 heteroatoms selected from nitrogen atom, oxygen atom and sulfur atom,

(j) 3- to 7-membered heterocycle comprising 1 to 3 heteroatoms selected from nitrogen atom, oxygen atom and sulfur atom, and being optionally substituted with alkyl or alkylcarbonyl,

(k) 3- to 7-membered heteroaryl comprising 1 to 3 heteroatoms selected from nitrogen atom, oxygen atom and sulfur atom.

(iv) —S—R⁵

wherein R⁵ is selected from aryl, aralkyl or 3- to 7-membered heteroaryl comprising 1 to 3 heteroatoms selected from nitrogen atom, oxygen atom and sulfur atom.

(v) —C(═O)—R⁶

wherein R⁶ is selected from hydroxy; alkoxy; amino; alkylamino being optionally substituted with cyano, hydroxy, carboxy, alkoxycarbonyl or aryl; arylamino; and 3- to 7-membered heterocycle comprising 1 to 3 heteroatoms selected from nitrogen atom, oxygen atom and sulfur atom, and being optionally substituted with hydroxy, carboxy, alkyl or alkoxycarbonyl.

(vi) 3- to 7-membered heteroaryl comprising 1 to 4 heteroatoms selected from nitrogen atom, oxygen atom and sulfur atom, said heteroaryl being optionally substituted with one or more substituents selected from alkyl; amino; alkoxy; alkoxycarbonyl; aryl; carboxy; and nitro where the substituent is unsubstituted or mono- or disubstituted with hydroxy, cyano, carboxy, alkoxy, formyl, alkylcarbonyl, alkoxycarbonyl, cycloalkyl, aryl or amino.

(vii) saturated or partially unsaturated, single or fused 3- to 10-membered heterocycle comprising 1 to 4 heteroatoms selected from nitrogen atom, oxygen atom and sulfur atom, said heterocycle being connected to the backbone through a ring member nitrogen and being optionally substituted with one or more substituents selected from the following groups:

(a) hydroxy, halogen, oxo, cyano, carboxy, hydroxyimino, hydrazidocarbonyl,

(b) amino being unsubstituted or independently mono- or disubstituted with alkyl (said alkyl is optionally substituted with hydroxy), formyl, alkylcarbonyl or alkoxycarbonyl,

(c) carbamoyl being unsubstituted or mono- or disubstituted with alkyl, cycloalkyl, hydroxy, hydroxyalkyl, aminoalkyl or aralkylsulfonyl,

(d) alkoxyimino being optionally substituted with aryl,

(e) alkyl being optionally substituted with hydroxy, halogen or amino (said amino is optionally substituted with alkylcarbonyl or alkoxycarbonyl),

(f) alkoxy,

(g) alkylcarbonyl being optionally substituted with hydroxy or halogen,

(h) alkoxycarbonyl being optionally substituted with alkylcarbonyloxy,

(i) alkylsulfonyl,

(j) alkylcarbonyloxy,

(k) alkylcarbonylamino being optionally substituted with hydroxy; amino; cyano; halogen; alkoxy; or 3- to 7-membered heteroaryl comprising 1 to 3 heteroatoms selected from nitrogen atom, oxygen atom and sulfur atom, and being optionally substituted with amino,

(l) cycloalkylcarbonylamino,

(m) 3- to 7-membered heteroarylcarbonylamino comprising 1 to 3 heteroatoms selected from nitrogen atom, oxygen atom and sulfur atom in the heteroaryl, and being optionally substituted with halogen,

(n) alkylsulfonylamino,

(O) aryl being optionally substituted with hydroxy,

(p) cycloalkyl,

(q) cycloalkylalkyl,

(r) aryloxycarbonylamino being optionally substituted with halogen,

(s) arylcarbonylamino being optionally substituted with halogen,

(t) cycloalkylaminocarbonylamino,

(u) arylaminocarbonylamino being optionally substituted with halogen,

(v) 3- to 7-membered heteroarylsulfonylaminocarbonylamino comprising 1 to 3 heteroatoms selected from nitrogen atom, oxygen atom and sulfur atom in the heteroaryl, and being optionally substituted with halogen, and

(w) 3- to 7-membered heterocyclylcarbonyl comprising 1 to 3 heteroatoms selected from nitrogen atom, oxygen atom and sulfur atom in the heterocycle;

(viii) azido.

Among the compounds of the Formula 1, preferable compounds are those wherein P, Q, R, T and X are defined as follows:

X represents N or C,

T represents N or C,

the ring Q represents a 3- to 7-membered aromatic ring which comprises 0 to 3 nitrogen atoms as ring members and is optionally benzo-fused, wherein the aromatic ring may be optionally substituted with oxo; alkyl; halogenoalkyl; hydroxyalkyl; alkoxy; aryl; or 3- to 7-membered heteroaryl comprising 1 to 3 heteroatoms selected from nitrogen atom, oxygen atom and sulfur atom,

P represents C₁-C₆-alkyl being optionally substituted with halogen, and

R represents a group selected from the following groups:

(i) —C₁-C₆-alkyl-R¹

wherein R¹ is selected from hydroxy; carboxy; carbamoyl; thiocarbamoyl; C₁-C₆-alkoxycarbonyl; C₆-C₁₀-aryloxy being optionally substituted with carboxy or C₁-C₆-alkoxycarbonyl; C₆-C₁₀-arylcarbonyloxy; 5- to 6-membered heteroaryl comprising 1 to 2 heteroatoms selected from nitrogen atom and sulfur atom, and being optionally substituted with carboxy or C₁-C₆-alkoxycarbonyl; and 5- to 6-membered heterocycle comprising 1 to 2 nitrogen atoms, and being optionally substituted with hydroxy.

(ii) —NR²R³

wherein each of R² and R³ is independently selected from hydrogen; C₁-C₆-alkyl being optionally substituted with amino (said amino is optionally substituted with carbamoyl), hydroxy, carboxy, hydroxy-C₆-C₁₀-aryl, C₁-C₆-alkoxy, C₁-C₆-alkoxycarbonyl, hydroxyl-C₁-C₆-alkoxy, or 5- to 6-membered heterocycle comprising 1 to 2 heteroatoms selected from nitrogen atom and sulfur atom (said heterocycle is optionally substituted with oxo or C₆-C₁₀-aryl-C₁-C₆-alkyl); C₃-C₆-cycloalkyl being optionally substituted with hydroxy or hydroxy-C₁-C₆-alkoxy; 4- to 6-membered heterocycle comprising 1 to 2 nitrogen atoms; C₆-C₁₀-aryl; C₆-C₁₀-aryl-C₁-C₆-alkyl; 5- to 6-membered heteroaryl comprising 1 to 2 heteroatoms selected from nitrogen atom and sulfur atom, and being optionally substituted with carboxy or C₁-C₆-alkoxycarbonyl.

(iii) —O—R⁴

wherein R⁴ is selected from the following groups:

(a) hydrogen,

(b) C₁-C₆-alkyl being optionally substituted with hydroxy; C₁-C₆-alkoxy; amino (said amino is optionally substituted with formyl or C₁-C₆-alkylcarbonyl); oxo; C₁-C₆-alkylcarbonyloxy-C₁-C₆-alkoxy; C₆-C₁₀-aryl being optionally substituted with halogen; 5- to 6-membered heteroaryl comprising 1 to 2 heteroatoms selected from nitrogen atom and oxygen, and being optionally substituted with carboxy-C₁-C₆-alkyl or C₁-C₆-alkoxycarbonyl-C₁-C₆-alkyl; 4- to 6-membered heterocycle comprising 1 to 2 heteroatoms selected from nitrogen atom and oxygen atom, and being optionally substituted with oxo; or 5- to 6-membered heteroarylcarbonylamino comprising 1 to 2 nitrogen atoms in the heteroaryl,

(c) C₃-C₆-cycloalkyl being optionally benzo-fused,

(d) C₁-C₆-alkylamino-C₁-C₆-alkyl being optionally substituted with C₁-C₆-alkoxycarbonyl or carboxy,

(e) C₃-C₆-cycloalkylcarbonylamino-C₁-C₆-alkyl,

(f) C₃-C₆-cycloalkylsulfonylamino-C₁-C₆-alkyl,

(g) C₁-C₆-alkylcarbonylamino-C₁-C₆-alkyl being optionally substituted with hydroxy, halogen, amino, C₁-C₆-alkoxy, C₁-C₆-alkylsulfonyl or aminosulfonyl,

(h) C₁-C₆-alkylsulfonylamino-C₁-C₆-alkyl being optionally substituted with halogen,

(i) C₆-C₁₀-aryl being optionally substituted with cyano; formyl; carboxy; C₁-C₆-alkoxycarbonyl; hydroxyl-C₁-C₆-alkyl; carboxy-C₁-C₆-alkyl; C₁-C₆-alkoxycarbonyl-C₁-C₆-alkyl; carboxy-C₁-C₆-alkoxy; C₁-C₆-alkoxycarbonyl-C₁-C₆-alkoxy; or 5- to 6-membered heterocycle comprising 1 to 2 nitrogen atoms,

(j) 4- to 6-membered heterocycle comprising 1 to 2 heteroatoms selected from nitrogen atom and oxygen atom, and being optionally substituted with C₁-C₆-alkyl,

(k) 5- to 6-membered heteroaryl comprising 1 to 2 nitrogen atoms.

(iv) —S—R⁵

wherein R⁵ is selected from C₆-C₁₀-aryl, C₆-C₁₀-aryl-C₁-C₆-alkyl or 5- to 6-membered heteroaryl comprising 1 to 2 nitrogen atoms.

(v) —C(═O)—R⁶

wherein R⁶ is selected from hydroxy; C₁-C₆-alkoxy; amino; C₁-C₆-alkylamino being optionally substituted with cyano, hydroxy, carboxy, C₁-C₆-alkoxycarbonyl or C₆-C₁₀-aryl; C₆-C₁₀-arylamino; and 5- to 6-membered heterocycle comprising 1 to 2 nitrogen atoms, and being optionally substituted with hydroxy, carboxy, C₁-C₆-alkyl or C₁-C₆-alkoxycarbonyl.

(vi) 5- to 6-membered heteroaryl comprising 2 to 4 heteroatoms selected from nitrogen atom, oxygen atom and sulfur atom, said heteroaryl being optionally substituted with one or more substituents selected from C₁-C₆-alkyl; amino; carboxy; C₁-C₆-alkoxy; C₁-C₆-alkoxycarbonyl; and C₆-C₁₀-aryl where the substituent is unsubstituted or mono- or disubstituted with hydroxy, cyano, carboxy, C₁-C₆-alkoxy, C₁-C₆-alkoxycarbonyl, C₃-C₆-cycloalkyl or C₆-C₁₀-aryl.

(vii) saturated or partially unsaturated, single or fused 3- to 10-membered heterocycle comprising 1 to 4 heteroatoms selected from nitrogen atom, oxygen atom and sulfur atom, said heterocycle being connected to the backbone through a ring member nitrogen and being optionally substituted with one or more substituents selected from the following groups:

(a) hydroxy, oxo, cyano, carboxy, hydroxyimino,

(b) amino being unsubstituted or mono- or disubstituted with C₁-C₆-alkyl (said alkyl is optionally substituted with hydroxy) or C₁-C₆-alkoxycarbonyl,

(c) carbamoyl being unsubstituted or mono- or disubstituted with C₁-C₆-alkyl, hydroxy, hydroxy-C₁-C₆-alkyl, amino-C₁-C₆-alkyl or C₆-C₁₀-aryl-C₁-C₆-alkylsulfonyl,

(d) C₁-C₆-alkoxyimino being optionally substituted with C₆-C₁₀-aryl,

(e) C₁-C₆-alkyl being optionally substituted with hydroxy, halogen or amino,

(f) C₁-C₆-alkoxy,

(g) C₁-C₆-alkylcarbonyl being optionally substituted with hydroxy or halogen,

(h) C₁-C₆-alkoxycarbonyl being optionally substituted with C₁-C₆-alkylcarbonyloxy,

(i) C₁-C₆-alkylsulfonyl,

(j) C₁-C₆-alkylcarbonyloxy,

(k) C₁-C₆-alkylcarbonylamino being optionally substituted with hydroxy; amino; cyano; halogen; C₁-C₆-alkoxy; or 5- to 6-membered heteroaryl comprising 1 to 2 heteroatoms selected from nitrogen atom and sulfur atom, and being optionally substituted with amino,

(l) C₃-C₆-cycloalkylcarbonylamino,

(m) 5- to 6-membered heteroarylcarbonylamino comprising 1 to 2 heteroatoms selected from nitrogen atom and oxygen atom in the heteroaryl,

(n) C₁-C₆-alkylsulfonylamino,

(o) C₆-C₁₀-aryl being optionally substituted with hydroxy,

(p) C₃-C₆-cycloalkyl, and

(q) C₃-C₆-cycloalkyl-C₁-C₆-alkyl.

Among the compounds of the Formula 1, especially preferable compounds are those wherein P, Q, R, T and X are defined as follows:

T represents N or C,

P represents C₁-C₄-alkyl being optionally substituted with fluorine,

the substituent

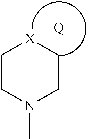

is optionally substituted with 1 to 2 substituents selected from the group consisting of oxo; C₁-C₄-alkyl being optionally substituted with fluorine; hydroxy-C₁-C₄-alkyl; C₁-C₄-alkoxy; phenyl; and furyl, and represents a heterocycle selected from the following structures:

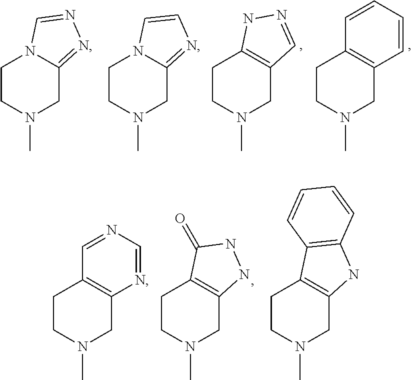

and

R represents a group selected from the following groups:

(i) —C₁-C₄-alkyl-R¹

wherein R¹ is selected from hydroxy; carboxy; carbamoyl; thiocarbamoyl; C₁-C₄-alkoxycarbonyl; phenyloxy being optionally substituted with carboxy or C₁-C₄-alkoxycarbonyl; benzoyloxy; thiazolyl comprising 1 to 2 heteroatoms selected from nitrogen atom and sulfur atom, and being optionally substituted with carboxy or C₁-C₄-alkoxycarbonyl; and pyrrolidinyl being optionally substituted with hydroxy.

(ii) —NR²R³

wherein each of R² and R³ is independently selected from hydrogen; C₁-C₄-alkyl being optionally substituted with amino (said amino is optionally substituted with carbamoyl), hydroxy, carboxy, hydroxyphenyl, C₁-C₄-alkoxy, C₁-C₄-alkoxycarbonyl, hydroxy-C₁-C₄-alkoxy, or pyrrolidinyl or thiazolidinyl being optionally substituted with oxo or benzyl; C₃-C₆-cycloalkyl being optionally substituted with hydroxy or hydroxy-C₁-C₄-alkoxy; 4- to 5-membered heterocycle comprising 1 nitrogen atom; pyrazolyl; phenyl; benzyl; pyrimidinyl; and thiazolyl being optionally substituted with carboxy or C₁-C₄-alkoxycarbonyl.

(iii) —O—R⁴

wherein R⁴ is selected from the following groups:

(a) hydrogen,

(b) C₁-C₄-alkyl being optionally substituted with hydroxy; C₁-C₄-alkoxy; amino (said amino is optionally substituted with formyl or C₁-C₄-alkylcarbonyl); oxo; C₁-C₄-alkylcarbonyloxy-C₁-C₄-alkoxy; phenyl being optionally substituted with halogen; pyridyl; oxazolyl being optionally substituted with carboxy-C₁-C₄-alkyl or C₁-C₄-alkoxycarbonyl-C₁-C₄-alkyl; 5-membered heterocycle comprising 1 heteroatom selected from nitrogen atom and oxygen atom, and being optionally substituted with oxo; or pyridylcarbonylamino,

(c) C₅-C₆-cycloalkyl being optionally benzo-fused,

(d) C₁-C₄-alkylamino-C₁-C₄-alkyl being optionally substituted with C₁-C₄-alkoxycarbonyl or carboxy,

(e) C₅-C₆-cycloalkylcarbonylamino-C₁-C₄-alkyl,

(f) C₅-C₆-cycloalkylsulfonylamino-C₁-C₄-alkyl,

(g) C₁-C₄-alkylcarbonylamino-C₁-C₄-alkyl being optionally substituted with hydroxy, halogen, amino, C₁-C₄-alkoxy, C₁-C₄-alkylsulfonyl or aminosulfonyl,

(h) C₁-C₄-alkylsulfonylamino-C₁-C₄-alkyl being optionally substituted with halogen,

(i) phenyl being optionally substituted with cyano; formyl; carboxy; C₁-C₄-alkoxycarbonyl; hydroxy-C₁-C₄-alkyl; carboxy-C₁-C₄-alkyl; C₁-C₄-alkoxycarbonyl-C₁-C₄-alkyl; carboxy-C₁-C₄-alkoxy; C₁-C₄-alkoxycarbonyl-C₁-C₄-alkoxy; or piperazinyl,

(j) tetrahydrofuryl; pyrrolidinyl being optionally substituted with C₁-C₄-alkyl; or acetidinyl,

(k) pyridyl.

(iv) —S—R⁵

wherein R⁵ is selected from phenyl, benzyl and pyrimidinyl.

(v) —C(═O)—R⁶

wherein R⁶ is selected from hydroxy; C₁-C₄-alkoxy; amino; C₁-C₄-alkylamino being optionally substituted with cyano, hydroxy, carboxy, C₁-C₄-alkoxycarbonyl or phenyl; phenylamino; and pyrrolidinyl, piperidinyl and piperazinyl being optionally substituted with hydroxy, carboxy, C₁-C₄-alkyl or C₁-C₄-alkoxycarbonyl.

(vi) oxadiazolyl, isoxadiazolyl, tetrazolyl, thiazolyl or pyrazolyl being optionally substituted with one or more substituents selected from C₁-C₄-alkyl; amino; carboxy; C₁-C₄-alkoxy; C₁-C₄-alkoxycarbonyl; and phenyl where the substituent is unsubstituted or mono- or disubstituted with hydroxy, cyano, carboxy, C₁-C₄-alkoxy, C₁-C₄-alkoxycarbonyl, C₃-C₆-cycloalkyl or phenyl.

(vii) heterocycle selected from the following structures and optionally substituted with one or more substituents selected from the groups (a) to (q):

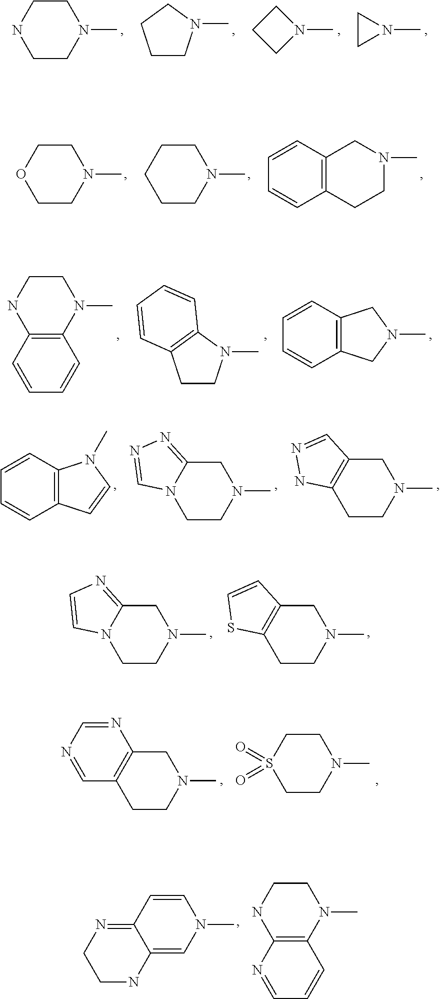

(a) hydroxy, oxo, cyano, carboxy, hydroxyimino,

(b) amino being unsubstituted or mono- or disubstituted with C₁-C₄-alkyl (said alkyl is optionally substituted with hydroxy) or C₁-C₄-alkoxycarbonyl,

(c) carbamoyl being unsubstituted or mono- or disubstituted with C₁-C₄-alkyl, hydroxy, hydroxy-C₁-C₄-alkyl, amino-C₁-C₄-alkyl or benzylsulfonyl,

(d) C₁-C₄-alkoxyimino being optionally substituted with phenyl,

(e) C₁-C₄-alkyl being optionally substituted with hydroxy, halogen or amino,

(f) C₁-C₄-alkoxy,

(g) C₁-C₄-alkylcarbonyl being optionally substituted with hydroxy or halogen,

(h) C₁-C₄-alkoxycarbonyl being optionally substituted with C₁-C₄-alkylcarbonyloxy,

(i) C₁-C₄-alkylsulfonyl,

(j) C₁-C₄-alkylcarbonyloxy,

(k) C₁-C₆-alkylcarbonylamino being optionally substituted with hydroxy; amino; cyano; halogen; C₁-C₄-alkoxy; or thiazolyl, imidazolyl or pyridyl being optionally substituted with amino,

(l) C₃-C₆-cycloalkylcarbonylamino,

(m) pyridylcarbonylamino or furylcarbonylamino,

(n) C₁-C₄-alkylsulfonylamino,

(o) phenyl being optionally substituted with hydroxy,

(p) C₃-C₆-cycloalkyl, and

(q) C₃-C₆-cycloalkyl-C₁-C₄-alkyl.

The most preferred compound among the compounds of Formula 1 according to the present invention can be selected from the following listed compounds:
4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propane-1,2-diol
7-[2-(4-methyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine
7-(2-piperazin-1-yl-6-propyl-thieno[2,3-d]pyrimidin-4-yl)-3-trifluoromethyl-5,6,7,8-tetrahydro-[1,2,4]triazolo[4,3-a]pyrazine
2-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanol
1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone
4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazine-1-carboxylic acid ethyl ester
7-[2-(4-ethanesulfonyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine
2-hydroxy-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone
2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-ethanol
(1-benzyl-pyrrolidin-3-ylmethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
C-{1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methylamine
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamine
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamine
7-(2-morpholin-4-yl-6-propyl-thieno[2,3-d]pyrimidin-4-yl)-3-trifluoromethyl-5,6,7,8-tetrahydro-[1,2,4]triazolo[4,3-a]pyrazine
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol
1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one
1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one oxime
1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-methyloxime
1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-benzyloxime

Acetic Acid
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester

Acetic Acid
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester
7-[2-(3-methoxy-pyrrolidin-1-yl)-6-propyl-thieno[2,3-d]pyrimidin-4-yl]-3-trifluoromethyl-5,6,7,8-tetrahydro-[1,2,4]triazolo[4,3-a]pyrazine
1-methyl-4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-ol
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ol
{1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-2-yl}-methanol
{1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-methanol
{1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-2-yl}-methanol
Cyclopentyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
Benzyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
(1S,2S,3S,5R)-3-(2-hydroxy-ethoxy)-5-[6-propyl-4-(3-trifluoromethyl-5,6-dihydro-8H-[1,2,4]triazolo[4,3-a]pyrazin-7-yl)-thieno[2,3-d]pyrimidin-2-ylamino]-cyclopentane-1,2-diol
2-{(2-hydroxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethanol
4-[6-propyl-4-(3-trifluoromethyl-5,6-dihydro-8H-[1,2,4]triazolo[4,3-a]pyrazin-7-yl)-thieno[2,3-d]pyrimidin-2-yl]-morpholin-2-one
Phenyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
[6-propyl-4-(3-trifluoromethyl-5,6-dihydro-8H-[1,2,4]triazolo[4,3-a]pyrazin-7-yl)-thieno[2,3-d]pyrimidin-2-yl]-pyrimidin-2-yl-amine
4-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-phenol
3-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-phenol
7-[2-(4-cyclopentyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine
7-[2-(4-cyclopentylmethyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine
7-[2-(4-cyclohexylmethyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine
3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid ethyl ester
3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid ethyl ester
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid isopropyl ester
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid
2,2-dimethyl-propionyloxymethyl ester
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid methyl ester
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid isopropylester
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid
2,2-dimethyl-propionyloxymethyl ester
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid methyl ester
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid
1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carbonitrile
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methyl ester
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid isopropylester
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid
2,2-dimethyl-propionyloxymethyl ester
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methyl ester
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid isopropylester
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid
2,2-dimethyl-propionyloxymethyl ester
Dimethyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
2-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline
6,7-dimethoxy-2-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline
1-ethyl-6,7-dimethoxy-2-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline
1-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-quinoxaline
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-ylamine
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine
7-[2-(2,3-dihydro-indol-1-yl)-6-propyl-thieno[2,3-d]pyrimidin-4-yl]-3-trifluoromethyl-5,6,7,8-tetrahydro-[1,2,4]triazolo[4,3-a]pyrazine
7-[2-(1,3-dihydro-isoindol-2-yl)-6-propyl-thieno[2,3-d]pyrimidin-4-yl]-3-trifluoromethyl-5,6,7,8-tetrahydro-[1,2,4]triazolo[4,3-a]pyrazine
7-(2-indol-1-yl-6-propyl-thieno[2,3-d]pyrimidin-4-yl)-3-trifluoromethyl-5,6,7,8-tetrahydro-[1,2,4]triazolo[4,3-a]pyrazine
7,7′-(6-propylthieno[2,3-d]pyrimidin-2,4-diyl)bis[3-(trifluoromethyl)-5,6,7,8-tetrahydro[1,2,4]triazolo[4,3-a]pyrazine]
7-[2-(5,6-dihydro-8H-[1,2,4]triazolo[4,3-a]pyrazin-7-yl)-6-propyl-thieno[2,3-d]pyrimidin-4-yl]-3-trifluoromethyl-5,6,7,8-tetrahydro-[1,2,4]triazolo[4,3-a]pyrazine
7-[6-propyl-2-(1,4,6,7-tetrahydro-pyrazolo[4,3-c]pyridin-5-yl)-thieno[2,3-d]pyrimidin-4-yl]-3-trifluoromethyl-5,6,7,8-tetrahydro-[1,2,4]triazolo[4,3-a]pyrazine
7-[2-(5,6-dihydro-8H-imidazo[1,2-a]pyrazin-7-yl)-6-propyl-thieno[2,3-d]pyrimidin-4-yl]-3-trifluoromethyl-5,6,7,8-tetrahydro-[1,2,4]triazolo[4,3-a]pyrazine
7-[6-propyl-2-(2-trifluoromethyl-5,6-dihydro-8H-imidazo[1,2-a]pyrazin-7-yl)-thieno[2,3-d]pyrimidin-4-yl]-3-trifluoromethyl-5,6,7,8-tetrahydro-[1,2,4]triazolo[4,3-a]pyrazine
7-[2-(6,7-dihydro-4H-thieno[3,2-c]pyridin-5-yl)-6-propyl-thieno[2,3-d]pyrimidin-4-yl]-3-trifluoromethyl-5,6,7,8-tetrahydro-[1,2,4]triazolo[4,3-a]pyrazine
2-methyl-7-[6-propyl-4-(3-trifluoromethyl-5,6-dihydro-8H-[1,2,4]triazolo[4,3-a]pyrazin-7-yl)-thieno[2,3-d]pyrimidin-2-yl]-4-trifluoromethyl-5,6,7,8-tetrahydro-pyrido[3,4-d]pyrimidine
3-(4-hydroxy-phenyl)-2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid
2,2,2-trifluoro-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone
Azetidin-3-yl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

Acetic Acid
1-[6-propyl-4-(3-trifluoromethyl-5,6-dihydro-8H-[1,2,4]triazolo[4,3-a]pyrazin-7-yl)-thieno[2,3-d]pyrimidin-2-yl]-azetidin-3-yl ester
1-[6-propyl-4-(3-trifluoromethyl-5,6-dihydro-8H-[1,2,4]triazolo[4,3-a]pyrazin-7-yl)-thieno[2,3-d]pyrimidin-2-yl]-azetidin-3-ol
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid amide
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methylamide
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid dimethylamide
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid hydroxyamide
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid
(2-hydroxy-ethyl)-amide
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid (2-amino-ethyl)-amide
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid amide
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid methylamide
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid dimethylamide
1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid hydroxyamide
1-[6-propyl-4-(3-trifluoromethyl-5,6-dihydro-8H-[1,2,4]triazolo[4,3-a]pyrazin-7-yl)-thieno[2,3-d]pyrimidin-2-yl]-aziridine-2-carboxylic acid methyl ester
Dimethyl-{1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amine
[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl-amine
2-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamino}-ethanol
2-hydroxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-amino-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-methoxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-cyano-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
3,3,3-trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide
N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2,2,2-trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-hydroxy-2-methyl-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

Cyclopropanecarboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide
3-hydroxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide
3-amino-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

Pyridine-2-carboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

Furan-2-carboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide
N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methanesulfonamide
4-amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol
4-amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one
4-amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one oxime
4-amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-methyl-oxime
{(2-hydroxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-acetic acid
2-(2-amino-thiazol-4-yl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-(1H-imidazol-4-yl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-pyridin-2-yl-acetamide
(2-ethoxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
2-{2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-ethoxy}-ethanol
7-[2-(1,1-dioxo-1lambda*6*-thiomorpholin-4-yl)-6-propyl-thieno[2,3-d]pyrimidin-4-yl]-3-trifluoromethyl-5,6,7,8-tetrahydro-[1,2,4]triazolo[4,3-a]pyrazine
(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine
[6-propyl-4-(3-trifluoromethyl-5,6-dihydro-8H-[1,2,4]triazolo[4,3-a]pyrazin-7-yl)-thieno[2,3-d]pyrimidin-2-yl]-(S)-pyrrolidin-3-yl-amine
C-phenyl-N-{1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-carbonyl}-methanesulfonamide
3-{2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-thiazolidin-4-one
(R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine
[6-propyl-4-(3-trifluoromethyl-5,6-dihydro-8H-[1,2,4]triazolo[4,3-a]pyrazin-7-yl)-thieno[2,3-d]pyrimidin-2-yl]-(R)-pyrrolidin-3-yl-amine
2-[6-propyl-4-(3-trifluoromethyl-5,6-dihydro-8H-[1,2,4]triazolo[4,3-a]pyrazin-7-yl)thieno[2,3-d]pyrimidin-2-ylamino]thiazole-4-carboxylic acid ethyl ester
2-[6-propyl-4-(3-trifluoromethyl-5,6-dihydro-8H-[1,2,4]triazolo[4,3-a]pyrazin-7-yl)thieno[2,3-d]pyrimidin-2-ylamino]thiazole-4-carboxylic acid
6-[6-propyl-4-(3-trifluoromethyl-5,6-dihydro-8H-[1,2,4]triazolo[4,3-a]pyrazin-7-yl)thieno[2,3-d]pyrimidin-2-yl]-2,3,4,6-tetrahydro-pyrido[3,4-b]pyrazine
1-[6-propyl-4-(3-trifluoromethyl-5,6-dihydro-8H-[1,2,4]triazolo[4,3-a]pyrazin-7-yl)thieno[2,3-d]pyrimidin-2-yl]-1,2,3,4-tetrahydro-pyrido[2,3-b]pyrazine
4-[6-propyl-4-(3-trifluoromethyl-5,6-dihydro-8H-[1,2,4]triazolo[4,3-a]pyrazin-7-yl)thieno[2,3-d]pyrimidin-2-yl]-1,2,3,4-tetrahydro-pyrido[2,3-b]pyrazine
4-[6-methyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
4-[6-ethyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
3-[6-ethyl-4-(3-trifluoromethyl-5,6-dihydro-8H-[1,2,4]triazolo[4,3-a]pyrazin-7-yl)-thieno[2,3-d]pyrimidin-2-ylamino]-propane-1,2-diol
(1S,2S,3R,5S)-3-[6-ethyl-4-(3-trifluoromethyl-5,6-dihydro-8H-[1,2,4]triazolo[4,3-a]pyrazin-7-yl)-thieno[2,3-d]pyrimidin-2-ylamino]-5-(2-hydroxy-ethoxy)-cyclopentane-1,2-diol
2-[6-ethyl-4-(3-trifluoromethyl-5,6-dihydro-8H-[1,2,4]triazolo[4,3-a]pyrazin-7-yl)-thieno[2,3-d]pyrimidin-2-ylamino]-ethanol
2-[[6-ethyl-4-(3-trifluoromethyl-5,6-dihydro-8H-[1,2,4]triazolo[4,3-a]pyrazin-7-yl)-thieno[2,3-d]pyrimidin-2-yl]-(2-hydroxy-ethyl)-amino]-ethanol
7-(6-ethyl-2-piperazin-1-yl-thieno[2,3-d]pyrimidin-4-yl)-3-trifluoromethyl-5,6,7,8-tetrahydro-[1,2,4]triazolo[4,3-a]pyrazine
N*1*-[6-ethyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine
[6-ethyl-4-(3-trifluoromethyl-5,6-dihydro-8H-[1,2,4]triazolo[4,3-a]pyrazin-7-yl)-thieno[2,3-d]pyrimidin-2-ylamino]-acetic acid
7-[6-ethyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine
2-[6-isopropyl-4-(3-trifluoromethyl-5,6-dihydro-8H-[1,2,4]triazolo[4,3-a]pyrazin-7-yl)-thieno[2,3-d]pyrimidin-2-ylamino]-ethanol
4-[6-isopropyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
7-[6-isopropyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine
4-[6-butyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
2-[6-butyl-4-(3-trifluoromethyl-5,6-dihydro-8H-[1,2,4]triazolo[4,3-a]pyrazin-7-yl)-thieno[2,3-d]pyrimidin-2-ylamino]-ethanol
7-[6-butyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine
4-[6-isobutyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one
(R)-1-[6-isobutyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol
4-[4-(3-trifluoromethyl-5,6-dihydro-8H-[1,2,4]triazolo[4,3-a]pyrazin-7-yl)-6-(3,3,3-trifluoro-propyl)-thieno[2,3-d]pyrimidin-2-yl]-piperazin-2-one
(R)-1-[4-(3-trifluoromethyl-5,6-dihydro-8H-[1,2,4]triazolo[4,3-a]pyrazin-7-yl)-6-(3,3,3-trifluoro-propyl)-thieno[2,3-d]pyrimidin-2-yl]-pyrrolidin-3-ol
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propane-1,2-diol
2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-propane-1,3-diol
2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethanol
7-[2-(2-methoxy-ethoxy)-6-propyl-thieno[2,3-d]pyrimidin-4-yl]-3-trifluoromethyl-5,6,7,8-tetrahydro-[1,2,4]triazolo[4,3-a]pyrazine
7-[6-propyl-2-(tetrahydro-furan-3-yloxy)-thieno[2,3-d]pyrimidin-4-yl]-3-trifluoromethyl-5,6,7,8-tetrahydro-[1,2,4]triazolo[4,3-a]pyrazine
7-[6-propyl-2-(tetrahydro-furan-2-ylmethoxy)-thieno[2,3-d]pyrimidin-4-yl]-3-trifluoromethyl-5,6,7,8-tetrahydro-[1,2,4]triazolo[4,3-a]pyrazine
7-[2-(1-methyl-pyrrolidin-3-yloxy)-6-propyl-thieno[2,3-d]pyrimidin-4-yl]-3-trifluoromethyl-5,6,7,8-tetrahydro-[1,2,4]triazolo[4,3-a]pyrazine
[6-propyl-4-(3-trifluoromethyl-5,6-dihydro-8H-[1,2,4]triazolo[4,3-a]pyrazin-7-yl)-thieno[2,3-d]pyrimidin-2-yloxy]-acetic acid methyl ester
[6-propyl-4-(3-trifluoromethyl-5,6-dihydro-8H-[1,2,4]triazolo[4,3-a]pyrazin-7-yl)-thieno[2,3-d]pyrimidin-2-yloxy]-acetic acid
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propan-1-ol
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid isopropyl ester
2,2-dimethyl-propionic acid 3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionyloxymethyl ester
7-[2-(3,3-dimethoxy-propoxy)-6-propyl-thieno[2,3-d]pyrimidin-4-yl]-3-trifluoromethyl-5,6,7,8-tetrahydro-[1,2,4]triazolo[4,3-a]pyrazine
7-(2-cyclopentyloxy-6-propyl-thieno[2,3-d]pyrimidin-4-yl)-3-trifluoromethyl-5,6,7,8-tetrahydro-[1,2,4]triazolo[4,3-a]pyrazine
7-(2-benzyloxy-6-propyl-thieno[2,3-d]pyrimidin-4-yl)-3-trifluoromethyl-5,6,7,8-tetrahydro-[1,2,4]triazolo[4,3-a]pyrazine
7-(2-butoxy-6-propyl-thieno[2,3-d]pyrimidin-4-yl)-3-trifluoromethyl-5,6,7,8-tetrahydro-[1,2,4]triazolo[4,3-a]pyrazine
{2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-oxazol-4-yl}-acetic acid ethyl ester
{2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-oxazol-4-yl}-acetic acid
7-[2-(oxazol-4-ylmethoxy)-6-propyl-thieno[2,3-d]pyrimidin-4-yl]-3-trifluoromethyl-5,6,7,8-tetrahydro-[1,2,4]triazolo[4,3-a]pyrazine
7-{6-propyl-2-[2-(2,3,5-trifluoro-phenyl)-ethoxy]-thieno[2,3-d]pyrimidin-4-yl}-3-trifluoromethyl-5,6,7,8-tetrahydro-[1,2,4]triazolo[4,3-a]pyrazine
7-[2-(indan-2-yloxy)-6-propyl-thieno[2,3-d]pyrimidin-4-yl]-3-trifluoromethyl-5,6,7,8-tetrahydro-[1,2,4]triazolo[4,3-a]pyrazine
7-[6-propyl-2-(pyridin-2-ylmethoxy)-thieno[2,3-d]pyrimidin-4-yl]-3-trifluoromethyl-5,6,7,8-tetrahydro-[1,2,4]triazolo[4,3-a]pyrazine
7-[6-propyl-2-(pyridin-3-ylmethoxy)-thieno[2,3-d]pyrimidin-4-yl]-3-trifluoromethyl-5,6,7,8-tetrahydro-[1,2,4]triazolo[4,3-a]pyrazine
7-[6-propyl-2-(pyridin-4-ylmethoxy)-thieno[2,3-d]pyrimidin-4-yl]-3-trifluoromethyl-5,6,7,8-tetrahydro-[1,2,4]triazolo[4,3-a]pyrazine
7-[2-(azetidin-3-yloxy)-6-propyl-thieno[2,3-d]pyrimidin-4-yl]-3-trifluoromethyl-5,6,7,8-tetrahydro-[1,2,4]triazolo[4,3-a]pyrazine
6-propyl-4-(3-trifluoromethyl-5,6-dihydro-8H-[1,2,4]triazolo[4,3-a]pyrazin-7-yl)-thieno[2,3-d]pyrimidin-2-ol
7-(2-phenoxy-6-propyl-thieno[2,3-d]pyrimidin-4-yl)-3-trifluoromethyl-5,6,7,8-tetrahydro-[1,2,4]triazolo[4,3-a]pyrazine
7-[6-propyl-2-(pyridin-3-yloxy)-thieno[2,3-d]pyrimidin-4-yl]-3-trifluoromethyl-5,6,7,8-tetrahydro-[1,2,4]triazolo[4,3-a]pyrazine
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid
2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester
2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid
4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester
4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid
7-[6-propyl-2-(pyridin-2-yloxy)-thieno[2,3-d]pyrimidin-4-yl]-3-trifluoromethyl-5,6,7,8-tetrahydro-[1,2,4]triazolo[4,3-a]pyrazine
3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzonitrile
4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzonitrile
{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-acetic acid methyl ester
{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-acetic acid
3-{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-propionic acid methyl ester
3-{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-propionic acid
{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenoxy}-acetic acid methyl ester
{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenoxy}-acetic acid
7-[2-(3-piperazin-1-yl-phenoxy)-6-propyl-thieno[2,3-d]pyrimidin-4-yl]-3-trifluoromethyl-5,6,7,8-tetrahydro-[1,2,4]triazolo[4,3-a]pyrazine
7-[2-(4-piperazin-1-yl-phenoxy)-6-propyl-thieno[2,3-d]pyrimidin-4-yl]-3-trifluoromethyl-5,6,7,8-tetrahydro-[1,2,4]triazolo[4,3-a]pyrazine
4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-benzaldehyde
{4-[6-propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-methanol
3-[6-ethyl-4-(3-trifluoromethyl-5,6-dihydro-8H-[1,2,4]triazolo[4,3-a]pyrazin-7-yl)-thieno[2,3-d]pyrimidin-2-yloxy]-propane-1,2-diol
2-[6-ethyl-4-(3-trifluoromethyl-5,6-dihydro-8H-[1,2,4]triazolo[4,3-a]pyrazin-7-yl)-thieno[2,3-d]pyrimidin-2-yloxy]-ethanol
7-(2-benzylsulfanyl-6-propyl-thieno[2,3-d]pyrimidin-4-yl)-3-trifluoromethyl-5,6,7,8-tetrahydro-[1,2,4]triazolo[4,3-a]pyrazine
7-(2-phenylsulfanyl-6-propyl-thieno[2,3-d]pyrimidin-4-yl)-3-trifluoromethyl-5,6,7,8-tetrahydro-[1,2,4]triazolo[4,3-a]pyrazine
7-[6-propyl-2-(pyrimidin-2-ylsulfanyl)-thieno[2,3-d]pyrimidin-4-yl]-3-trifluoromethyl-5,6,7,8-tetrahydro-[1,2,4]triazolo[4,3-a]pyrazine
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethylamino}-acetic acid ethyl ester
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethylamino}-acetic acid
(Cyclopropanecarbonyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-acetic acid ethyl ester
(Cyclopropanecarbonyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-acetic acid
2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethylamine

Cyclopropanecarboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide
2-hydroxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
2,2,2-trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
1-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-pyrrolidin-2-one
2-methoxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-methanesulfonamide
2-amino-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
2-methanesulfonyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-4-sulfamoyl-butylamide

Cyclopropanesulfonic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide
C,C,C-trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-methanesulfonamide

Pyridin-2-carboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide
2-[6-propyl-4-(3-trifluoromethyl-5,6-dihydro-8H-[1,2,4]triazolo[4,3-a]pyrazin-7-yl)thieno[2,3-d]pyrimidin-2-yl]thiazole-4-carboxylic acid ethyl ester
2-[6-propyl-4-(3-trifluoromethyl-5,6-dihydro-8H-[1,2,4]triazolo[4,3-a]pyrazin-7-yl)thieno[2,3-d]pyrimidin-2-yl]thiazole-4-carboxylic acid
(R)-2-[6-propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazole-4-carboxylic acid methyl ester
{(R)-2-[6-propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazol-4-yl}acetic acid isopropyl ester
{(R)-2-[6-propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazol-4-yl}acetic acid
{2-[6-propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}methanol
{2-[6-propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}acetic acid ethyl ester
{2-[6-propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}acetic acid
[6-propyl-4-(3-trifluoromethyl-5,6-dihydro-8H-[1,2,4]triazolo[4,3-a]pyrazin-7-yl)thieno[2,3-d]pyrimidin-2-yl]acetic acid ethyl ester
[6-propyl-4-(3-trifluoromethyl-5,6-dihydro-8H-[1,2,4]triazolo[4,3-a]pyrazin-7-yl)thieno[2,3-d]pyrimidin-2-yl]acetic acid
2-[6-propyl-4-(3-trifluoromethyl-5,6-dihydro-8H-[1,2,4]triazolo[4,3-a]pyrazin-7-yl)-thieno[2,3-d]pyrimidin-2-yl]-acetamide
2-[6-propyl-4-(3-trifluoromethyl-5,6-dihydro-8H-[1,2,4]triazolo[4,3-a]pyrazin-7-yl)-thieno[2,3-d]pyrimidin-2-yl]-thioacetamide
2-[6-propyl-4-(3-trifluoromethyl-5,6-dihydro-8H-[1,2,4]triazolo[4,3-a]pyrazin-7-yl)-thieno[2,3-d]pyrimidin-2-ylmethyl]-thiazole-4-carboxylic acid ethyl ester
2-[6-propyl-4-(3-trifluoromethyl-5,6-dihydro-8H-[1,2,4]triazolo[4,3-a]pyrazin-7-yl)-thieno[2,3-d]pyrimidin-2-ylmethyl]-thiazole-4-carboxylic acid
2-{2-[6-propyl-4-(3-trifluoromethyl-5,6-dihydro-8H-[1,2,4]triazolo[4,3-a]pyrazin-7-yl)-thieno[2,3-d]pyrimidin-2-yl]-thiazol-4-yl}-ethanol
4-[2-propyl-7-(3-trifluoromethyl-5,6-dihydro-8H-[1,2,4]triazolo[4,3-a]pyrazin-7-yl)-thiazolo[5,4-d]pyrimidin-5-yl]-piperazin-2-one
(S)-1-[2-propyl-7-(3-trifluoromethyl-5,6-dihydro-8H-[1,2,4]triazolo[4,3-a]pyrazin-7-yl)-thiazolo[5,4-d]pyrimidin-5-yl]-pyrrolidin-3-ylamine
(S)-1-[2-propyl-7-(3-trifluoromethyl-5,6-dihydro-8H-[1,2,4]triazolo[4,3-a]pyrazin-7-yl)-thiazolo[5,4-d]pyrimidin-5-yl]-piperidine-3-carboxylic acid ethyl ester
(S)-1-[2-propyl-7-(3-trifluoromethyl-5,6-dihydro-8H-[1,2,4]triazolo[4,3-a]pyrazin-7-yl)-thiazolo[5,4-d]pyrimidin-5-yl]-piperidine-3-carboxylic acid
6-propyl-4-(3-trifluoromethyl-5,6-dihydro-8H-[1,2,4]triazolo[4,3-a]pyrazin-7-yl)thieno[2,3-d]pyrimidine-2-carboxylic acid ethyl ester
6-propyl-4-(3-trifluoromethyl-5,6-dihydro-8H-[1,2,4]triazolo[4,3-a]pyrazin-7-yl)thieno[2,3-d]pyrimidine-2-carboxylic acid
6-propyl-4-(3-trifluoromethyl-5,6-dihydro-8H-[1,2,4]triazolo[4,3-a]pyrazin-7-yl)thieno[2,3-d]pyrimidine-2-carboxylic acid amide
{[6-ethyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid
6-ethyl-4-(3-trifluoromethyl-5,6-dihydro-8H-[1,2,4]triazolo[4,3-a]pyrazin-7-yl)thieno[2,3-d]pyrimidine-2-carboxylic acid (2-hydroxy-ethyl)amide
3-{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}propionic acid ethyl ester
3-{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}propionic acid
{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid ethyl ester
{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid
6-propyl-4-(3-trifluoromethyl-5,6-dihydro-8H-[1,2,4]triazolo[4,3-a]pyrazin-7-yl)thieno[2,3-d]pyrimidine-2-carboxylic acid (2,3-dihydroxy-propyl)amide
6-propyl-4-(3-trifluoromethyl-5,6-dihydro-8H-[1,2,4]triazolo[4,3-a]pyrazin-7-yl)thieno[2,3-d]pyrimidine-2-carboxylic acid (2-cyanoethyl)amide
(3-hydroxy-pyrrolidin-1-yl)-[6-propyl-4-(3-trifluoromethyl-5,6-dihydro-8H-[1,2,4]triazolo[4,3-a]pyrazin-7-yl)thieno[2,3-d]pyrimidin-2-yl]methanone
(4-methyl-piperazin-1-yl)-[6-propyl-4-(3-trifluoromethyl-5,6-dihydro-8H-[1,2,4]triazolo[4,3-a]pyrazin-7-yl)thieno[2,3-d]pyrimidin-2-yl]methanone
1-[6-propyl-4-(3-trifluoromethyl-5,6-dihydro-8H-[1,2,4]triazolo[4,3-a]pyrazin-7-yl)thieno[2,3-d]pyrimidine-2-carbonyl]piperidin-3-carboxylic acid ethyl ester
1-[6-propyl-4-(3-trifluoromethyl-5,6-dihydro-8H-[1,2,4]triazolo[4,3-a]pyrazin-7-yl)thieno[2,3-d]pyrimidine-2-carbonyl]piperidin-3-carboxylic acid
6-propyl-4-(3-trifluoromethyl-5,6-dihydro-8H-[1,2,4]triazolo[4,3-a]pyrazin-7-yl)thieno[2,3-d]pyrimidine-2-carboxylic acid phenylamide
6-propyl-4-(3-trifluoromethyl-5,6-dihydro-8H-[1,2,4]triazolo[4,3-a]pyrazin-7-yl)thieno[2,3-d]pyrimidine-2-carboxylic acid benzylamide
[6-ethyl-4-(3-trifluoromethyl-5,6-dihydro-8H-[1,2,4]triazolo[4,3-a]pyrazin-7-yl)thieno[2,3-d]pyrimidin-2-yl]methanol
6-propyl-4-(3-trifluoromethyl-5,6-dihydro-8H-[1,2,4]triazolo[4,3-a]pyrazin-7-yl)thieno[2,3-d]pyrimidin-2-yl]methanol
(R)-1-[6-ethyl-4-(3-trifluoromethyl-5,6-dihydro-8H-[1,2,4]triazolo[4,3-a]pyrazin-7-yl)thieno[2,3-d]pyrimidin-2-ylmethyl]pyrrolidin-3-ol
(R)-1-[6-propyl-4-(3-trifluoromethyl-5,6-dihydro-8H-[1,2,4]triazolo[4,3-a]pyrazin-7-yl)thieno[2,3-d]pyrimidin-2-ylmethyl]pyrrolidin-3-ol

Benzoic Acid
6-propyl-4-(3-trifluoromethyl-5,6-dihydro-8H-[1,2,4]triazolo[4,3-a]pyrazin-7-yl)thieno[2,3-d]pyrimidin-2-ylmethyl ester
7-(2-phenoxymethyl-6-propyl-thieno[2,3-d]pyrimidin-4-yl)-3-trifluoromethyl-5,6,7,8-tetrahydro-[1,2,4]triazolo[4,3-a]pyrazine
2-[6-propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid methyl ester
2-[6-propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid
3-[6-propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid
7-(2-(3-ethyl-[1,2,4]oxadiazol-5-yl)-6-propyl-thieno[2,3-d]pyrimidin-4-yl)-3-trifluoromethyl-5,6,7,8-tetrahydro-[1,2,4]triazolo[4,3-a]pyrazine
7-[2-(3-phenyl-[1,2,4]oxadiazol-5-yl)-6-propyl-thieno[2,3-d]pyrimidin-4-yl)-3-trifluoromethyl-5,6,7,8-tetrahydro-[1,2,4]triazolo[4,3-a]pyrazine
7-{2-[3-(2-methoxy-ethyl)-[1,2,4]oxadiazol-5-yl]-6-propyl-thieno[2,3-d]pyrimidin-4-yl}-3-trifluoromethyl-5,6,7,8-tetrahydro-[1,2,4]triazolo[4,3-a]pyrazine
2-{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,2,4]oxadiazol-3-yl}ethanol
7-{2-[3-(2,2-dimethoxy-ethyl)-[1,2,4]oxadiazol-5-yl]-6-propyl-thieno[2,3-d]pyrimidin-4-yl}-3-trifluoromethyl-5,6,7,8-tetrahydro-[1,2,4]triazolo[4,3-a]pyrazine
7-[2-(5-methyl-[1,3,4]oxadiazol-2-yl)-6-propyl-thieno[2,3-d]pyrimidin-4-yl)-3-trifluoromethyl-5,6,7,8-tetrahydro-[1,2,4]triazolo[4,3-a]pyrazine
7-[2-(5-phenyl-[1,3,4]oxadiazol-2-yl)-6-propyl-thieno[2,3-d]pyrimidin-4-yl)-3-trifluoromethyl-5,6,7,8-tetrahydro-[1,2,4]triazolo[4,3-a]pyrazine
{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetonitrile
7-[2-(5-methoxymethyl-[1,3,4]oxadiazol-2-yl)-6-propyl-thieno[2,3-d]pyrimidin-4-yl)-3-trifluoromethyl-5,6,7,8-tetrahydro-[1,2,4]triazolo[4,3-a]pyrazine
{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}methanol
7-{2-[5-(2-methoxyethyl)-[1,3,4]oxadiazol-2-yl]-6-propyl-thieno[2,3-d]pyrimidin-4-yl}-3-trifluoromethyl-5,6,7,8-tetrahydro-[1,2,4]triazolo[4,3-a]pyrazine
2-{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}ethanol
5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-ylamine
{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetic acid ethyl ester
{5-[6-propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetic acid
7-[2-(5-benzyl-[1,3,4]oxadiazol-2-yl)-6-propyl-thieno[2,3-d]pyrimidin-4-yl)-3-trifluoromethyl-5,6,7,8-tetrahydro-[1,2,4]triazolo[4,3-a]pyrazine
7-[2-(5-cyclohexylmethyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine
7-[6-propyl-2-(1H-tetrazol-5-yl)-thieno[2,3-d]pyrimidin-4-yl]-3-trifluoromethyl-5,6,7,8-tetrahydro-[1,2,4]triazolo[4,3-a]pyrazine
4-[2-ethyl-7-(3-trifluoromethyl-5,6-dihydro-8H-[1,2,4]triazolo[4,3-a]pyrazin-7-yl)-thiazolo[5,4-d]pyrimidin-5-yl]-piperazin-2-one
2-[2-ethyl-7-(3-trifluoromethyl-5,6-dihydro-8H-[1,2,4]triazolo[4,3-a]pyrazin-7-yl)-thiazolo[5,4-d]pyrimidin-5-ylamino]-ethanol
2-ethyl-5-(4-methyl-piperazin-1-yl)-7-(3-trifluoromethyl-5,6-dihydro-8H-[1,2,4]triazolo[4,3-a]pyrazin-7-yl)-thiazolo[5,4-d]pyrimidine
4-[6-ethyl-4-(3-pentafluoroethyl-5,6-dihydro-8H-[1,2,4]triazolo[4,3-a]pyrazin-7-yl)-thieno[2,3-d]pyrimidin-2-yl]-piperazin-2-one
3-[6-ethyl-4-(3-pentafluoroethyl-5,6-dihydro-8H-[1,2,4]triazolo[4,3-a]pyrazin-7-yl)-thieno[2,3-d]pyrimidin-2-ylamino]-propane-1,2-diol
3-[6-ethyl-4-(3-pentafluoroethyl-5,6-dihydro-8H-[1,2,4]triazolo[4,3-a]pyrazin-7-yl)-thieno[2,3-d]pyrimidin-2-yloxy]-propane-1,2-diol
4-[4-(3-pentafluoroethyl-5,6-dihydro-8H-[1,2,4]triazolo[4,3-a]pyrazin-7-yl)-6-propyl-thieno[2,3-d]pyrimidin-2-yl]-piperazin-2-one
3-pentafluoroethyl-7-(2-piperazin-1-yl-6-propyl-thieno[2,3-d]pyrimidin-4-yl)-5,6,7,8-tetrahydro-[1,2,4]triazolo[4,3-a]pyrazine
7-[2-(4-methyl-piperazin-1-yl)-6-propyl-thieno[2,3-d]pyrimidin-4-yl]-3-pentafluoroethyl-5,6,7,8-tetrahydro-[1,2,4]triazolo[4,3-a]pyrazine
2-[4-(3-pentafluoroethyl-5,6-dihydro-8H-[1,2,4]triazolo[4,3-a]pyrazin-7-yl)-6-propyl-thieno[2,3-d]pyrimidin-2-ylamino]-ethanol
3-[4-(3-pentafluoroethyl-5,6-dihydro-8H-[1,2,4]triazolo[4,3-a]pyrazin-7-yl)-6-propyl-thieno[2,3-d]pyrimidin-2-yloxy]-propane-1,2-diol
2-[4-(3-pentafluoroethyl-5,6-dihydro-8H-[1,2,4]triazolo[4,3-a]pyrazin-7-yl)-6-propyl-thieno[2,3-d]pyrimidin-2-yloxy]-ethanol
4-[4-(5,6-dihydro-8H-[1,2,4]triazolo[4,3-a]pyrazin-7-yl)-6-ethyl-thieno[2,3-d]pyrimidin-2-yl]-piperazin-2-one
3-[4-(5,6-dihydro-8H-[1,2,4]triazolo[4,3-a]pyrazin-7-yl)-6-ethyl-thieno[2,3-d]pyrimidin-2-yloxy]-propane-1,2-diol
4-[6-ethyl-4-(3-methyl-5,6-dihydro-8H-[1,2,4]triazolo[4,3-a]pyrazin-7-yl)-thieno[2,3-d]pyrimidin-2-yl]-piperazin-2-one
3-[6-ethyl-4-(3-methyl-5,6-dihydro-8H-[1,2,4]triazolo[4,3-a]pyrazin-7-yl)-thieno[2,3-d]pyrimidin-2-yloxy]-propane-1,2-diol
4-[4-(3-methyl-5,6-dihydro-8H-[1,2,4]triazolo[4,3-a]pyrazin-7-yl)-6-propyl-thieno[2,3-d]pyrimidin-2-yl]-piperazin-2-one
3-[4-(3-methyl-5,6-dihydro-8H-[1,2,4]triazolo[4,3-a]pyrazin-7-yl)-6-propyl-thieno[2,3-d]pyrimidin-2-yloxy]-propane-1,2-diol
2-[4-(3-methyl-5,6-dihydro-8H-[1,2,4]triazolo[4,3-a]pyrazin-7-yl)-6-propyl-thieno[2,3-d]pyrimidin-2-yloxy]-ethanol
3-[4-(3-methyl-5,6-dihydro-8H-[1,2,4]triazolo[4,3-a]pyrazin-7-yl)-6-propyl-thieno[2,3-d]pyrimidin-2-yloxy]-propane-1-ol
2-[4-(3-methyl-5,6-dihydro-8H-[1,2,4]triazolo[4,3-a]pyrazin-7-yl)-6-propyl-thieno[2,3-d]pyrimidin-2-yloxymethyl]-propane-1,3-diol
4-[6-ethyl-4-(3-phenyl-5,6-dihydro-8H-[1,2,4]triazolo[4,3-a]pyrazin-7-yl)-thieno[2,3-d]pyrimidin-2-yl]-piperazin-2-one
4-[4-(5,6-dihydro-8H-imidazo[1,2-a]pyrazin-7-yl)-6-ethyl-thieno[2,3-d]pyrimidin-2-yl]-piperazin-2-one
3-[4-(5,6-dihydro-8H-imidazo[1,2-a]pyrazin-7-yl)-6-ethyl-thieno[2,3-d]pyrimidin-2-yloxy]-propane-1,2-diol
4-[6-ethyl-4-(2-trifluoromethyl-5,6-dihydro-8H-imidazo[1,2-a]pyrazin-7-yl)-thieno[2,3-d]pyrimidin-2-yl]-piperazin-2-one
3-[6-ethyl-4-(2-trifluoromethyl-5,6-dihydro-8H-imidazo[1,2-a]pyrazin-7-yl)-thieno[2,3-d]pyrimidin-2-yloxy]-propane-1,2-diol
4-[6-ethyl-4-(1,4,6,7-tetrahydro-pyrazolo[4,3-c]pyridin-5-yl)-thieno[2,3-d]pyrimidin-2-yl]-piperazin-2-one
3-[6-ethyl-4-(1,4,6,7-tetrahydro-pyrazolo[4,3-c]pyridin-5-yl)-thieno[2,3-d]pyrimidin-2-yloxy]-propane-1,2-diol
4-[6-ethyl-4-(3-trifluoromethyl-1,4,6,7-tetrahydro-pyrazolo[4,3-c]pyridin-5-yl)-thieno[2,3-d]pyrimidin-2-yl]-piperazin-2-one
4-[4-(3,4-dihydro-1H-isoquinolin-2-yl)-6-ethyl-thieno[2,3-d]pyrimidin-2-yl]-piperazin-2-one
4-[4-(6,7-dimethoxy-3,4-dihydro-1H-isoquinolin-2-yl)-6-ethyl-thieno[2,3-d]pyrimidin-2-yl]-piperazin-2-one
4-[6-ethyl-4-(1,3,4,9-tetrahydro-beta-carbolin-2-yl)-thieno[2,3-d]pyrimidin-2-yl]-piperazin-2-one
4-[6-ethyl-4-(4-trifluoromethyl-5,8-dihydro-6H-pyrido[3,4-d]pyrimidin-7-yl)-thieno[2,3-d]pyrimidin-2-yl]-piperazin-2-one
4-[4-(2,4-bis-trifluoromethyl-5,8-dihydro-6H-pyrido[3,4-d]pyrimidin-7-yl)-6-ethyl-thieno[2,3-d]pyrimidin-2-yl]-piperazin-2-one
3-[4-(2,4-bis-trifluoromethyl-5,8-dihydro-6H-pyrido[3,4-d]pyrimidin-7-yl)-6-ethyl-thieno[2,3-d]pyrimidin-2-yloxy]-propane-1,2-diol
4-[4-(2,4-bis-trifluoromethyl-5,8-dihydro-6H-pyrido[3,4-d]pyrimidin-7-yl)-6-propylthieno[2,3-d]pyrimidin-2-yl]-piperazin-2-one
4-[6-ethyl-4-(2-methyl-4-trifluoromethyl-5,8-dihydro-6H-pyrido[3,4-d]pyrimidin-7-yl)-thieno[2,3-d]pyrimidin-2-yl]-piperazin-2-one
3-[6-ethyl-4-(2-methyl-4-trifluoromethyl-5,8-dihydro-6H-pyrido[3,4-d]pyrimidin-7-yl)-thieno[2,3-d]pyrimidin-2-yloxy]-propane-1,2-diol
4-[4-(2-methyl-4-trifluoromethyl-5,8-dihydro-6H-pyrido[3,4-d]pyrimidin-7-yl)-6-propyl-thieno[2,3-d]pyrimidin-2-yl]-piperazin-2-one
4-[6-ethyl-4-(2-phenyl-4-trifluoromethyl-5,8-dihydro-6H-pyrido[3,4-d]pyrimidin-7-yl)-thieno[2,3-d]pyrimidin-2-yl]-piperazin-2-one
4-[4-(2-phenyl-4-trifluoromethyl-5,8-dihydro-6H-pyrido[3,4-d]pyrimidin-7-yl)-6-propyl-thieno[2,3-d]pyrimidin-2-yl]-piperazin-2-one
4-[6-ethyl-4-(2-furan-3-yl-4-trifluoromethyl-5,8-dihydro-6H-pyrido[3,4-d]pyrimidin-7-yl)-thieno[2,3-d]pyrimidin-2-yl]-piperazin-2-one
3-[6-ethyl-4-(2-furan-3-yl-4-trifluoromethyl-5,8-dihydro-6H-pyrido[3,4-d]pyrimidin-7-yl)-thieno[2,3-d]pyrimidin-2-ylamino]-propane-1,2-diol
6-[6-ethyl-2-(3-oxo-piperazin-1-yl)-thieno[2,3-d]pyrimidin-4-yl]-2-methyl-1,2,4,5,6,7-hexahydro-pyrazolo[3,4-c]pyridin-3-one
6-[2-(2,3-dihydroxy-propoxy)-6-ethyl-thieno[2,3-d]pyrimidin-4-yl]-2-methyl-1,2,4,5,6,7-hexahydro-pyrazolo[3,4-c]pyridin-3-one
6-[6-ethyl-2-(3-oxo-piperazin-1-yl)-thieno[2,3-d]pyrimidin-4-yl]-2-(2-hydroxy-ethyl)-1,2,4,5,6,7-hexahydro-pyrazolo[3,4-c]pyridin-3-one
6-[6-ethyl-2-(3-oxo-piperazin-1-yl)-thieno[2,3-d]pyrimidin-4-yl]-2-phenyl-1,2,4,5,6,7-hexahydro-pyrazolo[3,4-c]pyridin-3-one
Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide
Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-acetamide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-butyramide
Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-isobutyramide
Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-acetamide
Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-butyramide
Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-isobutyramide
Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-acetamide
Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-butyramide
Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-isobutyramide

Hydrochloric Acid Salt of
1-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-butan-1-one

Hydrochloric Acid Salt of
2-Methyl-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-propan-1-one
N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
Acetic acid 4-acetylamino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester
N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide
N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide
N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2,2-dimethyl-propionamide
2-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
2-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-methyl-propionamide
3-Hydroxy-2-hydroxymethyl-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-methyl-propionamide
3-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2,2-dimethyl-propionamide
(S)-5-[6-Propyl-4-(3-trifluoromethyl-5,6-dihydro-8H-[1,2,4]triazolo[4,3-a]pyrazin-7-yl)-thieno[2,3-d]pyrimidin-2-yl]-hexahydro-pyrrolo[3,4-d]oxazol-2-one

Dihydrochloric Acid Salt of
1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3,4-diamine
N-{4-Acetylamino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-butyramide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-isobutyramide
2-Hydroxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
2-Hydroxy-2-methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide
3-Hydroxy-2-hydroxymethyl-2-methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide
3-Hydroxy-2,2-dimethyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide

Hydrochloric Acid Salt of
7-[6-Propyl-2-((R)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine

Hydrochloric Acid Salt of
7-[6-Propyl-2-((S)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine
Hydrochloric acid salt of N-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine
N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-acetamide
N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-butyramide
N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-isobutyramide
N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-acetamide
N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-butyramide
N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-isobutyramide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-acetamide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-butyramide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-isobutyramide
1-{3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-ethanone
1-{3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-butan-1-one
2-Methyl-1-{3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-propan-1-one
1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-ethanone
1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-butan-1-one
2-Methyl-1-{(S)-3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-propan-1-one
1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-ethanone
1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-butan-1-one
2-Methyl-1-{(R)-3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-propan-1-one
1-((S)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-ethanone
1-((S)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-butan-1-one
2-Methyl-1-((S)-3-{[6-propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-propan-1-one
1-((R)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-ethanone
1-((R)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-butan-1-one
2-Methyl-1-((R)-3-{[6-propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-propan-1-one
1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-ethanone
1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-butan-1-one
2-Methyl-1-{3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-propan-1-one
1-{(R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone
1-{(R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one
2-Methyl-1-{(R)-2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one
1-{(S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone
1-{(S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one
2-Methyl-1-{(S)-2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one
1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone
1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one
2-Methyl-1-{(R)-3-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one
1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone
1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one
2-Methyl-1-{(S)-3-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one
{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid ethyl ester
{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester
{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid ethyl ester
{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid 4-fluoro-phenyl ester

Cyclopentanecarboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide
{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid ethyl ester
{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid 4-fluoro-phenyl ester

Cyclopentanecarboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

Cyclohexanecarboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

Cyclohexanecarboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

5-Chloro-thiophene-2-carboxylic Acid
{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

5-Chloro-thiophene-2-carboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide
3,4,5-Trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-benzamide
3,4,5-Trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-benzamide
1-Cyclopentyl-3-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-urea
1-(3,4-Difluoro-phenyl)-3-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-urea
1-[(5-chloro-2-thienyl)sulfonyl]-3-[(3S)-1-[6-propyl-4-[3-(trifluoromethyl)-6,8-dihydro-5H-[1,2,4]triazolo[4,3-a]pyrazin-7-yl]thieno[2,3-d]pyrimidin-2-yl]pyrrolidin-3-yl]urea
{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester
{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester
{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester
N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide
N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide
N-Methyl-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
1-Methyl-3-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-urea

Pyrrolidine-1-carboxylic Acid
{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide
{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-urea
N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide
N-{4-Chloro-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
7-[2-(2-Methyl-3a,4,6,6a-tetrahydro-pyrrolo[3,4-d]oxazol-5-yl)-6-propyl-thieno[2,3-d]pyrimidin-4-yl]-3-trifluoromethyl-5,6,7,8-tetrahydro-[1,2,4]triazolo[4,3-a]pyrazine
N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-formamide
{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-carbamic acid methyl ester
1-[6-Propyl-4-(3-trifluoromethyl-5,6-dihydro-8H-[1,2,4]triazolo[4,3-a]pyrazin-7-yl)-thieno[2,3-d]pyrimidin-2-yl]-imidazolidin-2-one
7-[6-Propyl-2-(3a,4,6,6a-tetrahydro-pyrrolo[3,4-d]oxazol-5-yl)-thieno[2,3-d]pyrimidin-4-yl]-3-trifluoromethyl-5,6,7,8-tetrahydro-[1,2,4]triazolo[4,3-a]pyrazine
7-(2-Methoxy-6-propyl-thieno[2,3-d]pyrimidin-4-yl)-3-trifluoromethyl-5,6,7,8-tetrahydro-[1,2,4]triazolo[4,3-a]pyrazine
7-(2-Ethoxy-6-propyl-thieno[2,3-d]pyrimidin-4-yl)-3-trifluoromethyl-5,6,7,8-tetrahydro-[1,2,4]triazolo[4,3-a]pyrazine
7-(2-Azido-6-propyl-thieno[2,3-d]pyrimidin-4-yl)-3-trifluoromethyl-5,6,7,8-tetrahydro-[1,2,4]triazolo[4,3-a]pyrazine
6-Propyl-4-(3-trifluoromethyl-5,6-dihydro-8H-[1,2,4]triazolo[4,3-a]pyrazin-7-yl)-thieno[2,3-d]pyrimidin-2-ylamine
N-[6-Propyl-4-(3-trifluoromethyl-5,6-dihydro-8H-[1,2,4]triazolo[4,3-a]pyrazin-7-yl)-thieno[2,3-d]pyrimidin-2-yl]-acetamide
N-(2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-acetamide
N-(2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-formamide
(2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-carbamic acid methyl ester
7-(6-Propyl-2-[1,2,4]triazol-1-yl-thieno[2,3-d]pyrimidin-4-yl)-3-trifluoromethyl-5,6,7,8-tetrahydro-[1,2,4]triazolo[4,3-a]pyrazine
7-[6-Propyl-2-(2-[1,2,4]triazol-1-yl-ethoxy)-thieno[2,3-d]pyrimidin-4-yl]-3-trifluoromethyl-5,6,7,8-tetrahydro-[1,2,4]triazolo[4,3-a]pyrazine
[2-(1H-Imidazol-4-yl)-ethyl]-[6-propyl-4-(3-trifluoromethyl-5,6-dihydro-8H-[1,2,4]triazolo[4,3-a]pyrazin-7-yl)-thieno[2,3-d]pyrimidin-2-yl]-amine
2-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-imidazol-4-yl}-ethylamine
7-[2-(3-Nitro-pyrrole-1-yl)-6-propyl-thieno[2,3-d]pyrimidin-4-yl]-3-trifluoromethyl-5,6,7,8-tetrahydro-[1,2,4]triazolo[4,3-a]pyrazine
1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-ylamine
N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-yl}-acetamide
2-{[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrazin-2-yl-amino}-ethanol
3-(2-Hydroxy-ethyl)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-imidazolidine-2,4-dione
1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-ylamine
[6-Propyl-4-(3-trifluoromethyl-5,6-dihydro-8H-[1,2,4]triazolo[4,3-a]pyrazin-7-yl)-thieno[2,3-d]pyrimidin-2-yl]-(1H-pyrazol-3-yl)-amine
N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-yl}-acetamide
7-[2-(4-Methyl-pyrazol-1-yl)-6-propyl-thieno[2,3-d]pyrimidin-4-yl]-3-trifluoromethyl-5,6,7,8-tetrahydro-[1,2,4]triazolo[4,3-a]pyrazine
1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-4-ylamine
N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-4-yl}-acetamide
[6-Propyl-4-(3-trifluoromethyl-5,6-dihydro-8H-[1,2,4]triazolo[4,3-a]pyrazin-7-yl)-thieno[2,3-d]pyrimidin-2-yl]-(1H-pyrazol-4-yl)-amine
3-[4-(8-Oxo-3-trifluoromethyl-5,6-dihydro-8H-[1,2,4]triazolo[4,3-a]pyrazin-7-yl)-6-propyl-thieno[2,3-d]pyrimidin-2-yloxy]-propionic acid
7-{6-Propyl-2-[(S)-3-(pyrrolidine-1-carbonyl)-pyrrolidin-1-yl]-thieno[2,3-d]pyrimidin-4-yl}-3-trifluoromethyl-6,7-dihydro-5H-[1,2,4]triazolo[4,3-a]pyrazin-8-one
3-[6-Propionyl-4-(3-trifluoromethyl-5,6-dihydro-8H-[1,2,4]triazolo[4,3-a]pyrazin-7-yl)-thieno[2,3-d]pyrimidin-2-yloxy]-propionic acid
(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid butylamide
(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid butyl-methyl-amide
(S)-1-[6-Propyl-4-(3-trifluoromethyl-5,6-dihydro-8H-[1,2,4]triazolo[4,3-a]pyrazin7-yl)-thieno[2,3-d]pyrimidin-2-yl]-pyrrolidine-3-carboxylic acid cyclopentylamide
(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid cyclohexylmethyl-amide
N-Methyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide
N,N-Dimethyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide
3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-1-pyrrolidin-1-yl-propan-1-one
N-Cyclopentyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide
3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionamide
3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionitrile

Hydrochloric Acid Salt of
(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid hydrazide

Hydrochloric Acid Salt of
3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid hydrazide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-formamide
N-{1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide
N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-acetamide
N-{1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
N-{1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide
N-{1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid methyl ester
{1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester
{1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester
7-[2-(2-Fluoro-ethoxy)-6-propyl-thieno[2,3-d]pyrimidin-4-yl]-3-trifluoromethyl-5,6,7,8-tetrahydro-[1,2,4]triazolo[4,3-a]pyrazine
7-[6-Propyl-2-(2,2,2-trifluoro-ethoxy)-thieno[2,3-d]pyrimidin-4-yl]-3-trifluoromethyl-5,6,7,8-tetrahydro-[1,2,4]triazolo[4,3-a]pyrazine
1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-pyrrolidin-2-one
{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid methyl ester
N-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-acetamide
{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-carbamic acid methyl ester
N-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-acetamide
N-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-formamide
N-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-formamide
7-[6-Propyl-2-(3-pyrrol-1-yl-propoxy)-thieno[2,3-d]pyrimidin-4-yl]-3-trifluoromethyl-5,6,7,8-tetrahydro-[1,2,4]triazolo[4,3-a]pyrazine
N-Methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide
(2-Hydroxy-ethyl)-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester
Methyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester
N-(2-Hydroxy-ethyl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide
N-Methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
(2-Hydroxy-ethyl)-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester
3-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-oxazolidin-2-one

Acetic Acid
2-(acetyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-ethyl ester
N-(2-Hydroxy-ethyl)-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide
N-(2-Hydroxy-ethyl)-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide
3-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-propionitrile
3-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-propionamide
3-{2-[6-Propyl-4-(3-trifluoromethyl-5,6-dihydro-8H-[1,2,4]triazolo[4,3-a]pyrazin 7-yl)-thieno[2,3-d]pyrimidin-2-yloxy]-ethyl}-imidazolidine-2,4-dione
2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethanol
[6-Propyl-4-(3-trifluoromethyl-5,6-dihydro-8H-[1,2,4]triazolo[4,3-a]pyrazin-7-yl)-thieno[2,3-d]pyrimidin-2-yl]-carbamic acid methyl ester

Because the compounds according to the present invention may have a chiral carbon center, they can exist as R or S stereoisomer, racemate, diastereomer mixture and each diastereomer, and all of these stereoisomers and mixtures are included in the range of the present invention.

The compounds according to the present invention may also form pharmaceutically acceptable salts. These pharmaceutically acceptable salts include acid addition salts formed by acids which form nontoxic acid addition salts comprising pharmaceutically acceptable anion, for example, inorganic acids such as hydrochloric acid, sulfuric acid, nitric acid, phosphoric acid, hydrobromic acid, hydroiodic acid and the like; organic carboxylic acids such as tartaric acid, formic acid, citric acid, acetic acid, trichloroacetic acid, trifluoroacetic acid, gluconic acid, benzoic acid, lactic acid, fumaric acid, maleic acid and the like; sulfonic acids such as methanesulfonic acid, benzenesulfonic acid, p-toluenesulfonic acid or naphthalenesulfonic acid and the like; more preferably, acid addition salts formed by sulfuric acid, methanesulfonic acid or hydrohalogenic acid and the like. The compound of Formula 1 according to the present invention can be converted into their salts by conventional methods.

The compounds according to the present invention can include hydrate or solvate of the compound of the above Formula 1 or pharmaceutically acceptable salt thereof.

“Hydrate” means the compound or its salt of the present invention which contains stoichiometric or non-stoichiometric amount of water bound thereto by non-covalent intermolecular force.

“Solvate” means the compound or its salt of the present invention which containes stoichiometric or non-stoichiometric amount of solvent bound thereto by non-covalent intermolecular force. Preferable solvents are volatile, nontoxic, and/or suitable solvents to be administered to a human being.

Hereinafter, a method for preparing the compounds according to the present invention is explained based on the following reaction schemes in detail. However, the method explained by the following reaction schemes is only a part of the methods used in the present invention. For more specific preparation method, the preparation examples can be referred to, and the unit operation order, reagents, reacting conditions, solvents, etc. can be modified without limit if necessary.

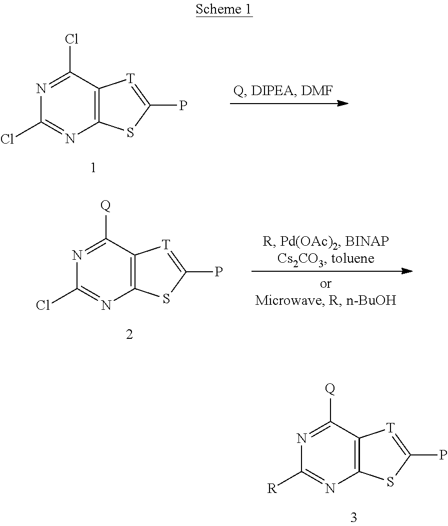

wherein, P, Q, R and T are the same as defined above.

In Scheme 1, the compound 2 is obtained by reacting the fused dichloro heterocyclic compound 1 with Q corresponding compound, for example, an amine or alcohol, in N,N-dimethylformamide. When R corresponding compound is amine, the compound 3 is obtained by reacting compound 2 with the R corresponding compound in a microwave reactor, and when R corresponding compound is alcohol, the compound 3 is obtained by reacting the compound 2 with the R corresponding compound through catalytic reaction using palladium acetate (II) and BINAP.

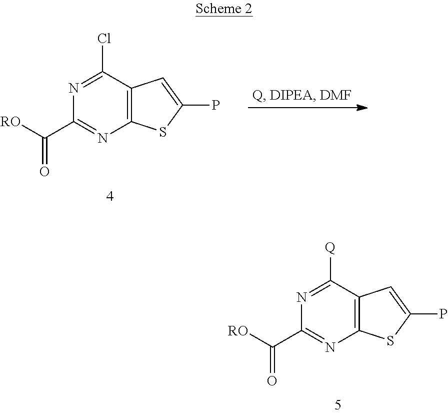

In Scheme 2, the compound 5 is obtained by reacting the compound 4 with Q corresponding compound in dimethylformamide.

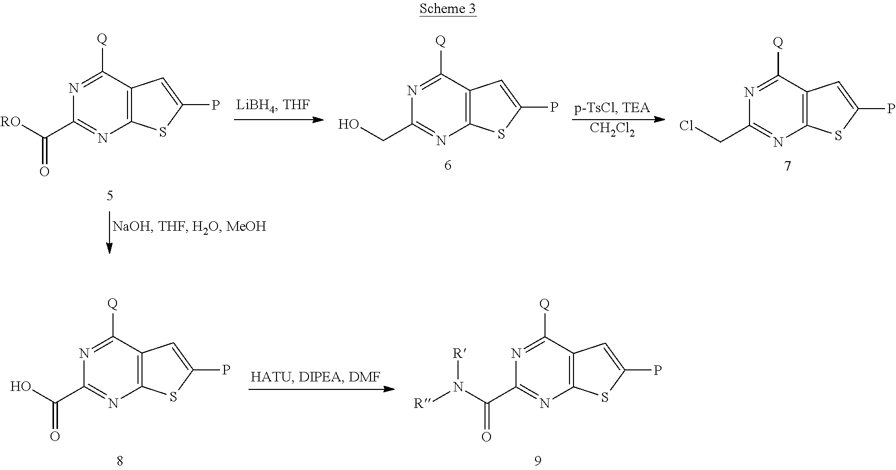

In Scheme 3, the alcohol compound 6 is obtained by reacting the ester compound 5 with a reducing agent, for example, lithium borohydride or lithium aluminiumhydride, in tetrahydrofuran solution, and the compound 7 is obtained by reacting the compound 6 with para-toluenesulfonylchloride. The compound 8 is obtained by hydrolyzing the compound 5 with sodium hydroxide, and the compound 9 is obtained by reacting the compound 8 with amine, for example, primary or secondary amine, using the binding agent HATU in N,N-dimethylformamide.

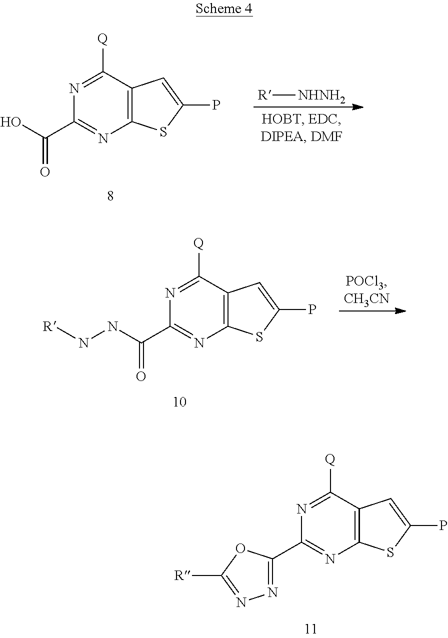

In Scheme 4, the compound 10 is obtained by reacting the compound 8 with mono-substituted hydrazine using the binding agents HOBT and EDC, and the compound 11 is obtained by reacting the compound 10 with phosphorous oxychloride in acetonitrile.

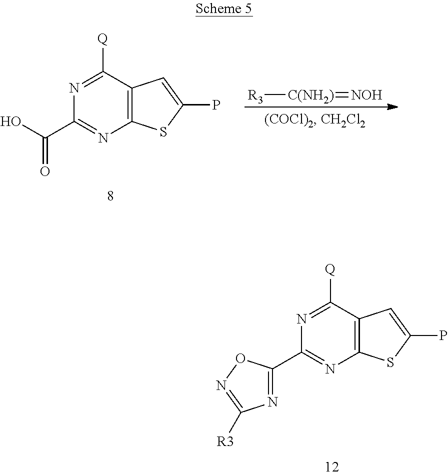

In Scheme 5, the compound 12 is obtained by reacting the compound 8 with N-hydroxy alkylamidine, for example, N-hydroxy-propionamidine, in oxalyl chloride and methylene chloride.

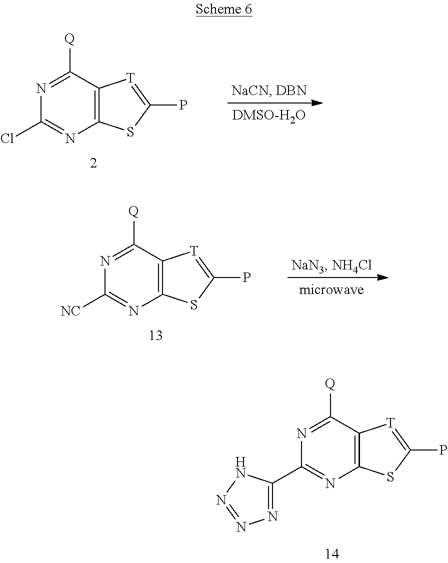

In Scheme 6, the tetrazole-substituted compound 14 can be obtained by cyanating the compound 2 followed by using sodium azide.

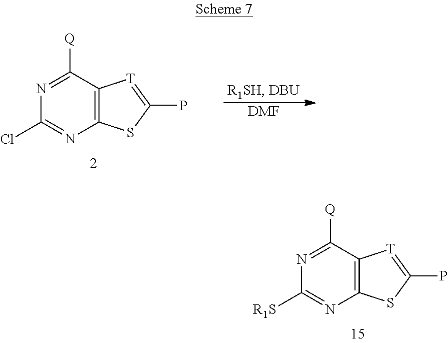

The thiol-substituted compound 15 is obtained by using thiol and DBU.

Column chromatography is generally used to isolate the reaction mixture, and the final compound can be further isolated by recrystallization, or normal or reverse phase HPLC (Waters, Delta Pack, 300×50 mmI.D., C18 5 μm, 100 A). In case of the isolation using recryatallization or HPLC, the compound can be obtained as a form of trifluoroacetic acid salt, and ion exchange resin can be used to obtain hydrochloric acid salt.

After completing the reaction according to the above method of the present invention, the product can be isolated and purified by conventional post-treatment methods such as chromatography, recrystallization and the like.

The compound of Formula 1 according to the present invention has a broad spectrum of inhibitory activity against platelet aggregation as demonstrated by the following experimental results. Especially, it acts against P2Y12 which is a platelet ADP-receptor, so that it can act antagonistically against ADP and accordingly inhibit thrombus formation.

Thus, the present invention provides a pharmaceutical composition for inhibiting platelet aggregation, specifically for preventing and/or treating vascular disease related to platelet aggregation in blood vessels such as peripheral blood vessels and cardiac blood vessels, comprising the compound of Formula 1 or pharmaceutically acceptable salt thereof as an active ingredient, together with the pharmaceutically acceptable carrier.

More specifically, the composition of the present invention has a preventive or therapeutic effect for inhibition of circulatory disease closely related to thrombus formation resulting from platelet aggregation; acceleration of platelet separation; antithrombotic; reconstructive surgery including skins and muscle flap; mechanically induced platelet activation in the organism, such as cardiopulmonary bypass and extra-corporeal membranous oxygenation; or primary arterial thrombotic complication of atherosclerosis, such as stable or unstable angina pectoris, thrombotic or embolic apoplexy, transient ischemic attack, peripheral vascular disease, myocardial infarction with or without thrombolytic agent, arterial complication resulting from the involvement of atherosclerotic disease, such as transplant surgery of angioplasia, including coronary angioplasia, endarterectomy stent indwelled coronary vascular and other vascular, thrombotic complication of surgery or mechanical injury such as tissue saving accompanying with trauma resulting from accidents or surgery, disseminated intravascular coagulation, thrombotic thrombocytopenic purpura, hemolytic uremic syndrome, thrombotic complication of sepsis, adult respiratory distress syndrome (ARDS), heparin induced thrombocytopenia and preeclampsia, state with disseminated thrombotic, platelet consumption ingredient such as eclampsia, deep venous thrombosis, intravenous thrombosis such as intravenous embolic disease, hematological states such as myeloproliferative disorder including thrombocytopenia, drepanocytemia, shunt occlusion in kidney dialysis or plasmapheresis, etc., secondary thrombosis of vascular injury or inflammation such as vasculitis, arteritis, glomerulonephritis, inflammatory bowel disease and organ transplantation rejection, status hemicranicus, Raynaud's phenomenon, states that can cause original inflammatory disease progress in vessel walls such as platelet atheroma plague formation and progression, coarctation and restenosis, other inflammatory states such as asthma wherein platelet and platelet-induced factors are included in the progress of immunological disease, central nervous disease, or tumor growth and extension.

More specifically, the composition of the present invention has a preventive or therapeutic effect for phlebothrombosis, thrombophlebitis, arterial embolism, coronary artery and cerebral artery thrombosis, myocardial infarction, stroke, cerebral embolism, kidney embolism, pulmonary embolism, thrombotic apoplexy, transient ischemic attack, peripheral vascular disease and stable and unstable angina pectoris.

The composition of the present invention may be formulated into various pharmaceutical administration forms according to the purpose. In preparing the pharmaceutical composition according to the present invention, an effective amount of the compound of Formula 1 or its pharmaceutically acceptable salt is mixed with a pharmaceutically acceptable carrier that may take a wide variety of forms depending on the formulation to be prepared.

The present composition for inhibiting platelet aggregation may be formulated as a parenteral injection, or percutaneous or oral preparation depending on its application purpose. It is preferable to formulate the composition in a unit dosage form for easy administration and uniform dosage.

For the oral preparation, any usual pharmaceutical carrier may be used. For example, water, glycols, oils, alcohols and the like may be used for oral liquid preparations such as suspensions, syrups, elixirs and solutions; or starches, sugars, kaolin, lubricants, binders, disintegrating agents and the like may be used for solid preparations such as powders, pills, capsules and tablets.

For the parenteral preparation, sterile water is usually used as the carrier, and other ingredients such as solubility aids may also be used. Injections, for example, sterilized aqueous or oily suspension for injection, can be prepared according to the known procedure using suitable dispersing agent, wetting agent, or suspending agent. Solvents that can be used for preparing injections include water, Ringer's fluid, and isotonic NaCl solution, and sterilized fixing oil may also be used conventionally as the solvent or suspending media. Any non-stimulative fixing oil including mono-, di-glyceride may be used for this purpose. Fatty acid such as oleic acid may also be used for injections. For the percutaneous preparation, the carrier may include a penetration enhancing agent and/or a suitable wetting agent, optionally combined with suitable additives having no skin irritation. Said additives may facilitate the administration through the skin and/or assist preparation of a desired composition. These percutaneous preparations are administered in various manners, e.g., as a transdermal patch, a spot-on, or an ointment.

When the compound of the present invention is used for clinical purpose, it is preferably administered to the subject patient in an amount ranging from 0.001 to 100 mg per kg of body weight a day and the total daily dosage may be administered once or over several times. However, in some cases, a lower administration dosage than the above range may be more preferable, whereas in other cases, a higher administration dosage than the above range may be used if there is no harmful side effect. In addition, specific administration dosage for an individual patient can be varied with specific compound used, body weight, sex, hygienic condition, or diet of subject patient, time or method of administration, excretion rate, mixing ratio of agent, severity of disease to be treated, etc.

### Advantageous Effects of Invention

The compound of Formula 1 according to the present invention has a broad spectrum of inhibitory activity against platelet aggregation as demonstrated by the following experimental results. Especially, it acts against P2Y12, which is a platelet ADP-receptor, so that it can act antagonistically against ADP and accordingly inhibit thrombus formation.

Therefore, the present invention provides a pharmaceutical composition for inhibiting platelet aggregation, specifically a preventive and therapeutic composition for vascular disease, such as peripheral vascular and cardiovascular disease, related to platelet aggregation, comprising the compound of Formula 1 or pharmaceutically acceptable salt thereof as an active ingredient, together with the pharmaceutically acceptable carrier.

### MODE FOR THE INVENTION

The present invention is further explained in detail through the following Preparation Examples and Examples. However, the scope of the present invention is not limited thereto.

### Preparation Example 1-1-1

### 2,4-Dichloro-6-propyl-thieno[2,3-d]pyrimidine

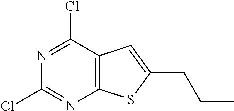

The synthesis was carried out according to a known method (WO 2006/079916).

### Preparation Example 1-1-2

### 3-Trifluoromethyl-5,6,7,8-tetrahydro-[1,2,4]triazolo[4,3-a]pyrazine; hydrochloride

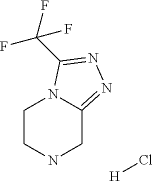

The synthesis was carried out according to a known method (Organic Letters 2005, 7(6), 1039-1042).

### Preparation Example 1-1-3

### 7-(2-Chloro-6-propyl-thieno[2,3-d]pyrimidin-4-yl)-3-trifluoromethyl-5,6,7,8-tetrahydro-[1,2,4]triazolo[4,3-a]pyrazine

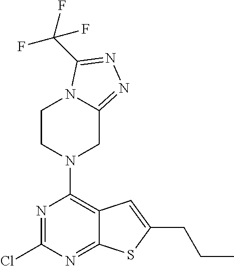

The compound obtained in Preparation Example 1-1-1 (225 mg, 0.91 mmol) and the compound obtained in Preparation Example 1-1-2 (250 mg, 1.09 mmol) were diluted in N,N-dimethylformamide (5 mL) and diisopropylethylamine (353 mg, 2.73 mmol) was added thereto, and the mixture was stirred for 16 hours. The reaction mixture was distilled under reduced pressure, diluted with dichloromethane, and washed with water. The organic layer was dried with anhydrous magnesium sulfate, distilled under reduced pressure, and then solidified and rinsed with diethyl ether to obtain the title compound (289 mg, 79%).

¹H NMR (400 MHz, CDCl₃); δ 7.04 (1H, s), 5.36 (2H, s), 4.38 (4H, m), 2.89 (2H, t), 1.77 (2H, m), 1.02 (3H, t)

### Example 1-1

### 4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

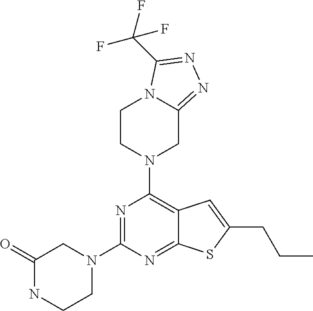

The compound obtained in Preparation Example 1-1-3 (161 mg, 0.4 mmol) and piperiazin-2-one (80 mg, 0.8 mmol) were diluted in butanol (3 mL), and the mixture was heated in a microwave reactor to 150° C. and stirred for 2 hours. The reaction solution was cooled to room temperature and distilled under reduced pressure, diluted with dichloromethane, and washed with water. The organic layer was dried with anhydrous magnesium sulfate, distilled under reduced pressure, and then solidified and rinsed with diethyl ether to obtain the title compound (176 mg, 94%).

¹H NMR (400 MHz, CDCl₃); δ 6.83 (1H, s), 6.60 (1H, br s), 5.22 (2H, s), 4.42 (2H, s), 4.35 (2H, t), 4.24 (2H, t), 4.05 (2H, t), 3.48 (2H, m), 2.79 (2H, t), 1.72 (2H, m), 1.00 (3H, t)

### Example 1-2

### 3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-propane-1,2-diol

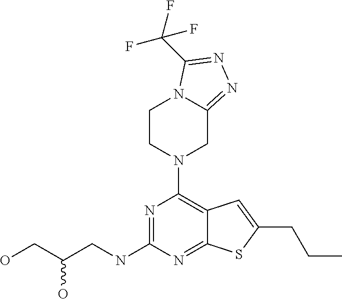

Except that 3-amino-propan-1,2-diol (18 mg, 0.2 mmol) was used instead of piperiazin-2-one, the same procedure as in Example 1-1 was carried out to obtain the title compound (44 mg, 96%).

¹H NMR (400 MHz, CDCl₃); δ 6.77 (1H, s), 5.67 (1H, br s), 5.14 (2H, s), 4.31 (2H, t), 4.20 (2H, t), 3.88 (1H, m), 3.70˜3.50 (6H, m), 2.74 (2H, t), 1.69 (2H, m), 0.98 (3H, t)

### Example 1-3

### 7-[2-(4-Methyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine

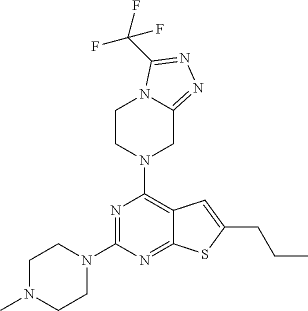

Except that 1-methylpiperazine (20 mg, 0.2 mmol) was used instead of piperiazin-2-one, the same procedure as in Example 1-1 was carried out to obtain the title compound (44 mg, 94%).

¹H NMR (400 MHz, CDCl₃); δ 6.77 (1H, s), 5.16 (2H, s), 4.34 (2H, t), 4.19 (2H, t), 3.82 (4H, t), 2.77 (2H, t), 2.47 (4H, t), 2.35 (3H, s), 1.73 (2H, m), 0.99 (3H, t)

### Preparation Example 1-4-1

### 4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazine-1-carboxylic acid tert-butyl ester

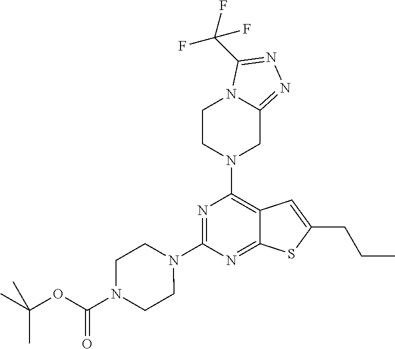

Except that piperiazine-1-carboxylic acid tert-butyl ester (37 mg, 0.2 mmol) was used instead of piperiazin-2-one, the same procedure as in Example 1-1 was carried out to obtain the title compound (37 mg, 67%).

¹H NMR (400 MHz, CDCl₃); δ 6.79 (1H, s), 5.17 (2H, s), 4.34 (2H, t), 4.21 (2H, t), 3.78 (4H, t), 3.49 (4H, t), 2.78 (2H, t), 1.73 (2H, m), 1.49 (9H, s), 1.00 (3H, t)

### Example 1-4

### 7-(2-piperazin-1-yl-6-propyl-thieno[2,3-d]pyrimidin-4-yl)-3-trifluoromethyl-5,6,7,8-tetrahydro-[1,2,4]triazolo[4,3-a]pyrazine

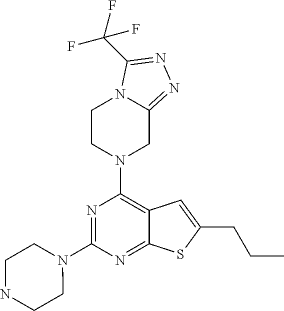

The compound obtained in Preparation Example 1-4-1 (37 mg, 0.067 mmol) was dissolved in 4.0 M hydrochloric acid dioxane solution (5 mL) and stirred for 1 hour. The resulting mixture was distilled under reduced pressure to remove the solvent, and then solidified and rinsed with diethyl ether to obtain the title compound (30 mg, 86%).

¹H NMR (400 MHz, DMSO, d₆); δ 9.11 (2H, br s), 7.28 (1H, s), 5.15 (2H, s), 4.36 (2H, t), 4.23 (2H, t), 3.94 (4H, br s), 3.14 (4H, br s), 2.79 (2H, t), 1.66 (2H, m), 0.95 (3H, t)

### Example 1-5

### 2-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanol

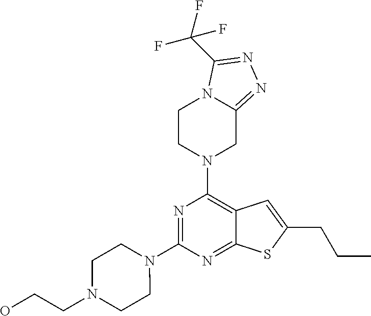

Except that 2-piperiazin-1-yl-ethanol (26 mg, 0.2 mmol) was used instead of piperiazin-2-one, the same procedure as in Example 1-1 was carried out to obtain the title compound (46 mg, 92%).

¹H NMR (400 MHz, CDCl₃); δ 6.78 (1H, s), 5.16 (2H, s), 4.34 (2H, t), 4.20 (2H, t), 3.82 (4H, t), 3.68 (2H, t), 2.78 (2H, t), 2.64˜2.54 (6H, m), 1.72 (2H, m), 0.99 (3H, t)

### Example 1-6

### 1-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone

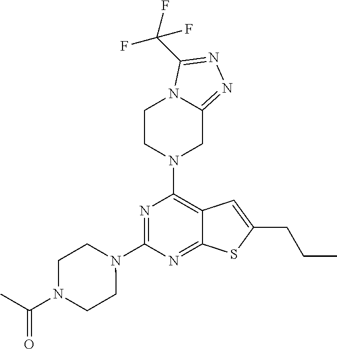

Except that 1-piperiazin-1-yl-ethanone (26 mg, 0.2 mmol) was used instead of piperiazin-2-one, the same procedure as in Example 1-1 was carried out to obtain the title compound (48 mg, 98%).

¹H NMR (400 MHz, CDCl₃); δ 6.80 (1H, s), 5.18 (2H, s), 4.35 (2H, t), 4.21 (2H, t), 3.83 (2H, t), 3.79 (2H, t), 3.69 (2H, t), 3.53 (2H, t), 2.79 (2H, t), 2.16 (3H, s), 1.73 (2H, m), 1.00 (3H, t)

### Example 1-7

### 4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazine-1-carboxylic acid ethyl ester

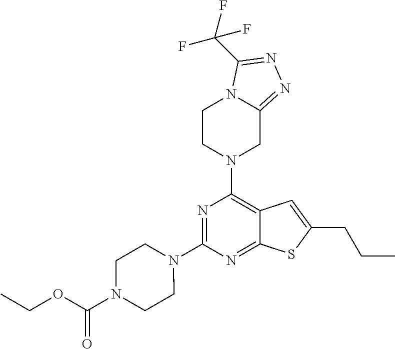

Except that piperazine-1-carboxylic acid ethyl ester (32 mg, 0.2 mmol) was used instead of piperiazin-2-one, the same procedure as in Example 1-1 was carried out to obtain the title compound (49 mg, 94%).

¹H NMR (400 MHz, CDCl₃); δ 6.79 (1H, s), 5.17 (2H, s), 4.34 (2H, t), 4.26 ˜4.12 (4H, m), 3.79 (2H, t), 3.55 (2H, t), 2.78 (2H, t), 1.72 (2H, m), 1.30 (3H, t), 0.98 (3H, t)

### Example 1-8

### 7-[2-(4-Ethanesulfonyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine

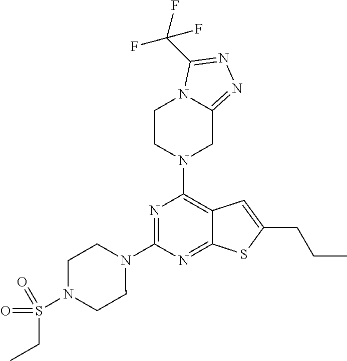

Except that 1-ethanesulfonyl-piperazine (36 mg, 0.2 mmol) was used instead of piperiazin-2-one, the same procedure as in Example 1-1 was carried out to obtain the title compound (44 mg, 82%).

¹H NMR (400 MHz, CDCl₃): δ 6.80 (1H, s), 5.18 (2H, s), 4.35 (2H, t), 4.22 (2H, t), 3.90 (4H, t), 3.35 (4H, t), 2.97 (2H, q), 2.79 (2H, t), 1.73 (2H, m), 1.39 (3H, t), 0.99 (3H, t)

### Preparation Example 1-9-1

### 4-(2-Hydroxy-acetyl)-piperazine-1-carboxylic acid tert-butyl ester

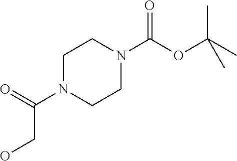

piperazine-1-carboxylic acid tert-butyl ester (186 mg, 1.0 mmol), hydroxyacetic acid (91 mg, 1.2 mmol) and HATU (456 mg, 1.2 mmol) were dissolved in N,N-dimethylformamide (5.0 mL) and diisopropylethylamine (646 mg, 5.0 mmol) was added thereto, and the mixture was stirred for 16 hours. The reaction mixture was distilled under reduced pressure, diluted with dichloromethane, and washed with water. The organic layer was dried with anhydrous magnesium sulfate, distilled under reduced pressure, and purified by column chromatography using 1:2 mixture solvent of hexane and ethyl acetate to obtain the title compound (180 mg, 74%).

¹H NMR (400 MHz, CDCl₃); δ 4.18 (2H, s), 3.65 (2H, t), 4.46 (4H, t), 3.5 (2H, t), 2.80 (1H, s), 1.47 (9H, s)

### Example 1-9

### 2-Hydroxy-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone

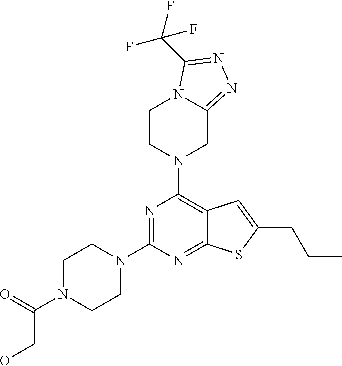

Except that the compound obtained in Preparation Example 1-9-1 (49 mg, 0.2 mmol) instead of piperiazin-2-one was dissolved in 4.0 M hydrochloric acid dioxane solution (5 mL) and stirred for 1 hour and the resulting mixture was distilled under reduced pressure to remove the solvent, the same procedure as in Example 1-1 was carried out to obtain the title compound (23 mg, 45%).

¹H NMR (400 MHz, CDCl₃); δ 6.80 (1H, s), 5.18 (2H, s), 4.34 (2H, t), 4.21 (4H, t), 3.85 (4H, t), 3.75 (2H, t), 3.63 (1H, br s), 3.35 (2H, t), 2.79 (2H, q), 1.73 (2H, m), 1.00 (3H, t)

### Example 1-10

### 2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethanol

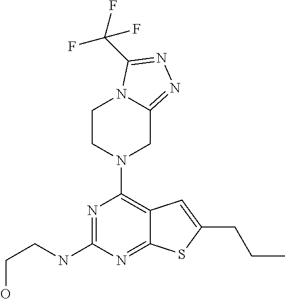

Except that ethanolamine (12 mg, 0.2 mmol) was used instead of piperiazin-2-one, the same procedure as in Example 1-1 was carried out to obtain the title compound (42 mg, 98%).

¹H NMR (400 MHz, CDCl₃); δ 6.79 (1H, s), 5.37 (1H, t), 5.17 (2H, s), 4.33 (2H, t), 4.20 (2H, t), 3.83 (2H, t), 3.59 (2H, q), 2.77 (2H, t), 1.71 (2H, m), 0.99 (3H, t)

### Example 1-11

### (1-Benzyl-pyrrolidin-3-ylmethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

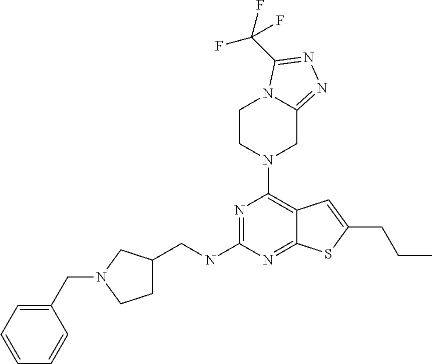

Except that C-(1-benzyl-pyrrolidin-3-yl)-methylamine (38 mg, 0.2 mmol) was used instead of piperiazin-2-one, the same procedure as in Example 1-1 was carried out to obtain the title compound (20 mg, 36%).

¹H NMR (400 MHz, CDCl₃); δ 7.40˜7.18 (5H, m), 6.78 (1H, s), 5.44 (1H, br s), 5.18 (2H, s), 4.32 (2H, t), 4.19 (2H, t), 3.65 (2H, m), 3.41 (2H, t), 2.77 (2H, t), 2.66 (1H, m), 2.51 (2H, m), 2.05 (1H, m), 1.72 (2H, m), 1.62 (1H, m), 0.99 (3H, t)

### Preparation Example 1-12-1

### {1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-carbamic acid tert-butyl ester

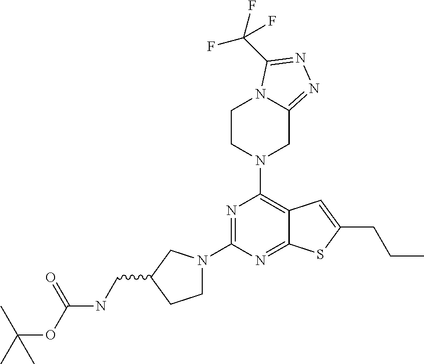

Except that pyrrolidin-3-ylmethyl-carbamic acid tert-butyl ester (40 mg, 0.2 mmol) was used instead of piperiazin-2-one, the same procedure as in Example 1-1 was carried out to obtain the title compound (35 mg, 74%).

¹H NMR (400 MHz, CDCl₃); δ 6.76 (1H, s), 5.18 (2H, s), 4.71 (1H, br s), 4.35 (2H, t), 4.20 (2H, t), 3.74 (2H, m), 3.53 (1H, m), 3.27 (2H, m), 3.16 (1H, m), 2.77 (2H, t), 2.50 (1H, m), 2.12 (1H, m), 1.78˜1.68 (3H, m), 1.46 (9H, s), 0.99 (3H, t)

### Example 1-12

### C-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methylamine

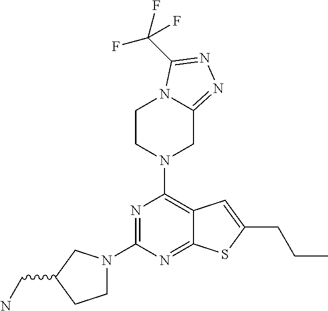

Except that the compound obtained in Preparation Example 1-12-1 (35 mg, 0.075 mmol) was used instead of the compound obtained in Preparation Example 1-4-1, the same procedure as in Example 1-4 was carried out to obtain the title compound (37 mg, 93%).

¹H NMR (400 MHz, DMSO, d₆); δ 8.16 (3H, br s), 7.32 (1H, s), 5.23 (2H, s), 4.68 (2H, br s), 4.30 (2H, br s), 3.78˜3.30 (4H, m), 2.93 (2H, t), 2.80 (2H, t), 2.59 (1H, s), 2.16 (1H, m), 1.82 (1H, m), 1.67 (2H, m), 0.95 (3H, t)

### Preparation Example 1-13-1

### {(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid tert-butyl ester

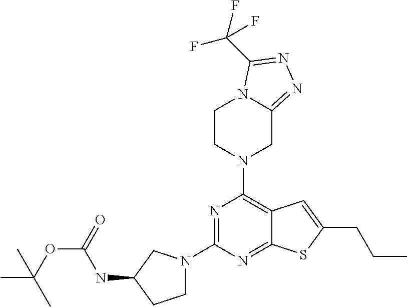

Except that (R)-pyrrolidin-3-yl-carbamic acid tert-butyl ester (37 mg, 0.2 mmol) was used instead of piperiazin-2-one, the same procedure as in Example 1-1 was carried out to obtain the title compound (39 mg, 87%).

¹H NMR (400 MHz, CDCl₃); δ 6.78 (1H, s), 5.18 (2H, s), 4.72 (1H, br s) 4.34 (2H, t), 4.32 (1H, m), 4.20 (2H, t), 3.84 (1H, dd), 3.73˜3.42 (4H, m), 2.77 (2H, t), 2.24 (1H, m), 1.94 (1H, m), 1.72 (2H, m), 1.45 (9H, s), 0.99 (3H, t)

### Example 1-13

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamine

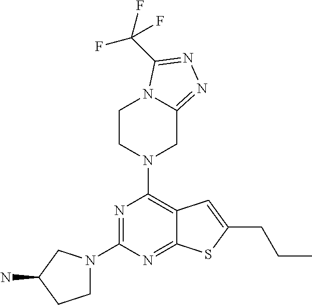

Except that the compound obtained in Preparation Example 1-13-1 (39 mg, 0.086 mmol) was used instead of the compound obtained in Preparation Example 1-4-1, the same procedure as in Example 1-4 was carried out to obtain the title compound (39 mg, 87%).

¹H NMR (400 MHz, DMSO, d₆); δ 8.42 (3H, br s), 7.33 (1H, s), 5.23 (2H, s), 4.38 (2H, br s), 4.30 (2H, br s), 4.0˜3.62 m), 3.48 (1H, m), 2.81 (2H, t), 2.31 (1H, m), 2.14 (1H, m), 1.67 (1H, m), 0.95 (3H, t)

### Preparation Example 1-14-1

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid tert-butyl ester

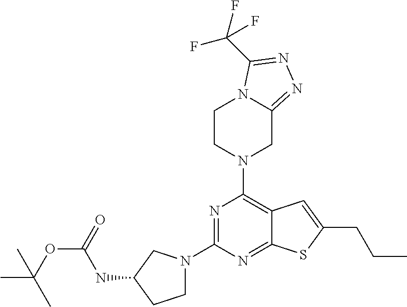

Except that (S)-pyrrolidin-3-yl-carbamic acid tert-butyl ester (37 mg, 0.2 mmol) was used instead of piperiazin-2-one, the same procedure as in Example 1-1 was carried out to obtain the title compound (39 mg, 87%).

¹H NMR (400 MHz, CDCl₃); δ 6.78 (1H, s), 5.18 (2H, s), 4.72 (1H, br s) 4.34 (2H, t), 4.32 (1H, m), 4.20 (2H, t), 3.84 (1H, dd), 3.73˜3.42 (4H, m), 2.77 (2H, t), 2.24 (1H, m), 1.94 (1H, m), 1.72 (2H, m), 1.45 (9H, s), 0.99 (3H, t)

### Example 1-14

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamine

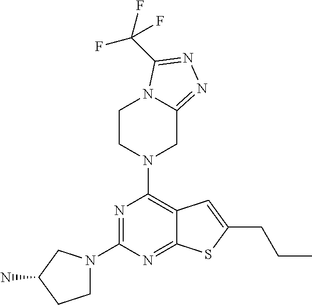

Except that the compound obtained in Preparation Example 1-14-1 (39 mg, 0.086 mmol) was used instead of the compound obtained in Preparation Example 1-4-1, the same procedure as in Example 1-4 was carried out to obtain the title compound (39 mg, 87%).

¹H NMR (400 MHz, DMSO, d₆); δ 8.42 (3H, br s), 7.33 (1H, s), 5.23 (2H, s), 4.38 (2H, br s), 4.30 (2H, br s), 4.0˜3.62 (4H, m), 3.48 (1H, m), 2.81 (2H, t), 2.31 (1H, m), 2.14 (1H, m), 1.67 (1H, m), 0.95 (3H, t)

### Example 1-15

### 7-(2-Morpholin-4-yl-6-propyl-thieno[2,3-d]pyrimidin-4-yl)-3-trifluoromethyl-5,6,7,8-tetrahydro-[1,2,4]triazolo[4,3-a]pyrazine

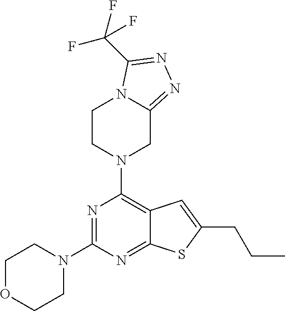

Except that morpholine (17 mg, 0.2 mmol) was used instead of piperiazin-2-one, the same procedure as in Example 1-1 was carried out to obtain the title compound (44 mg, 98%).

¹H NMR (400 MHz, CDCl₃); δ6.79 (1H, s), 5.17 (2H, s), 4.34 (2H, t), 4.20 (2H, t), 3.77 (8H, s), 2.78 (2H, t), 1.72 (2H, m), 1.00 (3H, t)

### Example 1-16

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol

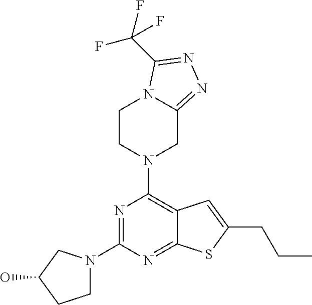

Except that (S)-pyrrolidin-3-ol (17 mg, 0.2 mmol) was used instead of piperiazin-2-one, the same procedure as in Example 1-1 was carried out to obtain the title compound (44 mg, 98%).

¹H NMR (400 MHz, CDCl₃); δ 6.75 (1H, s), 5.15 (2H, s), 4.58 (1H, br s), 4.32 (2H, t), 4.18 (2H, t), 3.76˜3.62 (4H, m), 2.76 (2H, t), 2.26 (1H, br s), 2.18˜2.02 (2H, m), 1.71 (2H, m), 0.99 (3H, t)

### Example 1-17

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol

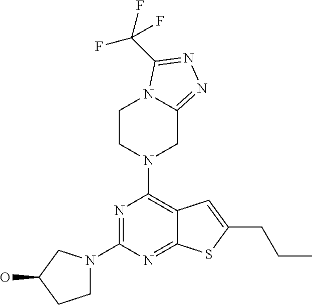

Except that (R)-pyrrolidin-3-ol (17 mg, 0.2 mmol) was used instead of piperiazin-2-one, the same procedure as in Example 1-1 was carried out to obtain the title compound (45 mg, 100%).

¹H NMR (400 MHz, CDCl₃); δ 6.75 (1H, s), 5.15 (2H, s), 4.58 (1H, br s), 4.32 (2H, t), 4.18 (2H, t), 3.76˜3.62 (4H, m), 2.76 (2H, t), 2.26 (1H, br s), 2.18-2.02 (2H, m), 1.71 (2H, m), 0.99 (3H, t)

### Example 1-18

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one

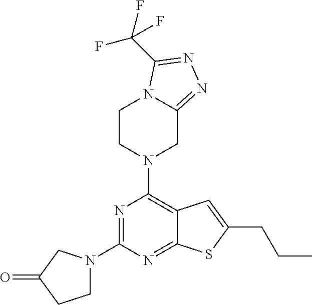

The compound obtained in Example 1-17 (113 mg, 0.25 mmol) was dissolved in dichloromethane (5 mL) and N-methylmorpholine N-oxide (44 mg, 0.375 mmol) and TPAP (4 mg, 0.013 mmol) was added thereto, and the mixture was stirred for 1 hour. The reaction mixture was distilled under reduced pressure to remove the solvent, and purified by column chromatography using 3:97 mixture solvent of methanol and dichloromethane to obtain the title compound (67 mg, 59%).

¹H NMR (400 MHz, CDCl₃); δ 6.82 (1H, s), 5.22 (2H, s), 4.36 (2H, t), 4.24 (2H, t), 4.06-3.59 (4H, m), 2.80 (2H, t), 2.72 (2H, t), 1.74 (2H, m), 1.01 (3H, t)

### Example 1-19

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one oxime

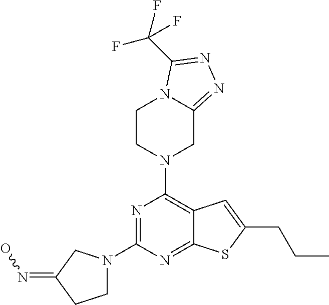

The compound obtained in Example 1-18 (23 mg, 0.05 mmol), hydroxylamine; hydrochloric acid salt (4 mg, 0.06 mmol) and sodium carbonate (3 mg, 0.03 mmol) were diluted in ethanol (2 mL) and water (1 mL), and the mixture was stirred for 16 hours. The mixture was distilled under reduced pressure to remove the solvent, and purified by column chromatography using 5:95 mixture solvent of methanol and dichloromethane to obtain the title compound (21 mg, 91%).

¹H NMR (100 MHz, CDCl₃+CD₃OD)); δ 6.84 (1H, s), 5.22 (2H, s), 4.42˜4.21 m), 3.84 (2H, dd), 2.94 (1H, t), 2.81 (3H, m), 1.73 (2H, m), 1.00 (3H, t)

### Example 1-20

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-methyloxime

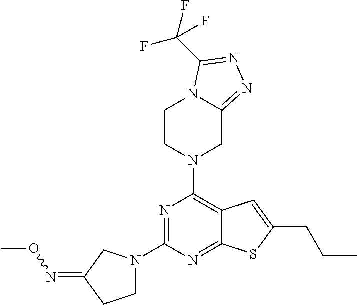

Except that methoxylamine; hydrochloric acid salt (5 mg, 0.056 mmol) was used instead of hydroxylamine; hydrochloric acid salt, the same procedure as in Example 1-19 was carried out to obtain the title compound (20 mg, 91%).

¹H NMR (400 MHz, CDCl₃); δ 6.80 (1H, d), 5.20 (2H, s), 4.50˜4.18 (6H, m), 3.92 (3H, s), 3.82 (2H, m), 2.90-2.75 (4H, m), 1.72 (2H, m), 1.00 (3H, t)

### Example 1-21

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-benzyloxime

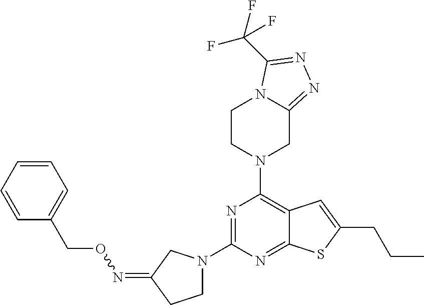

Except that O-benzylhydroxylamine; hydrochloric acid salt (9 mg, 0.056 mmol) was used instead of hydroxylamine; hydrochloric acid salt, the same procedure as in Example 1-19 was carried out to obtain the title compound (24 mg, 89%).

¹H NMR (400 MHz, CDCl₃); δ 7.42˜7.28 (5H, m), 6.79 (1H, d), 5.19 (2H, d), 5.14 (2H, d), 4.40˜4.16 (6H, m), 3.82 (2H, dd), 2.91 (1H, t), 2.87˜2.73 (3H, m), 1.72 (2H, m), 1.00 (3H, t)

### Example 1-22

### Acetic Acid

### (S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester

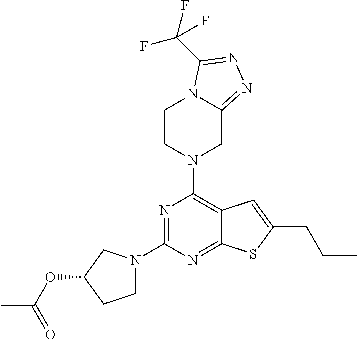

The compound obtained in Example 1-16 (41 mg, 0.09 mmol) was dissolved in dichloromethane (5 mL) and cooled to 0° C., and diisopropylethylamine (35 mg, 0.27 mmol) and acetic anhydride (18 mg, 0.18 mmol) were added thereto. A catalytic amount of 4-dimethylaminopyridine was added and the temperature was raised to room temperature and the mixture was stirred for 16 hours. The mixture was distilled under reduced pressure to remove the solvent, and purified by column chromatography using 3:2 mixture solvent of hexane and ethyl acetate to obtain the title compound (42 mg, 95%).

¹H NMR (400 MHz, CDCl₃); δ 6.78 (1H, s), 5.41 (1H, m), 5.19 (2H, s), 4.35 (2H, t), 4.25 (2H, t), 3.86-3.59 (4H, m), 2.77 (2H, t), 2.28-2.12 (2H, m), 2.05 (3H, s), 1.72 (2H, m), 0.99 (3H, t)

### Example 1-23

### Acetic Acid

### (R)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester

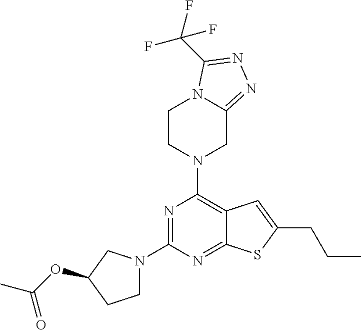

Except that the compound obtained in Example 1-17 (42 mg, 0.093 mmol) was used instead of the compound obtained in Example 1-16, the same procedure as in Example 1-22 was carried out to obtain the title compound (43 mg, 93%).

¹H NMR (400 MHz, CDCl₃); δ 6.78 (1H, s), 5.41 (1H, m), 5.19 (2H, s), 4.35 (2H, t), 4.25 (2H, t), 3.86˜3.59 (4H, m), 2.77 (2H, t), 2.28˜2.12 (2H, m), 2.05 (3H, s), 1.72 (2H, m), 0.99 (3H, t)

### Preparation Example 1-24-1

### 3-Methoxy-pyrrolidine-1-carboxylic acid tert-butyl ester

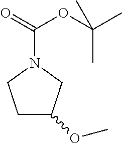

3-Hydroxy-pyrrolidine-1-carboxylic acid tert-butyl ester (187 mg, 1.0 mmol) was dissolved in N,N-dimethylformamide (5 mL) and cooled to 0° C., and 60% sodium hydride 60 mg (1.5 mmol) was added thereto and the mixture was stirred for 15 minutes. Iodomethane (284 mg, 2.0 mmol) was added thereto, and the mixture was stirred at room temperature for 1 hour and 30 minutes and cooled to 0° C., and then the reaction was terminated with water. The reaction mixture was diluted with ethyl acetate and washed with water and brine. The organic layer was dried with anhydrous magnesium sulfate, distilled under reduced pressure, and purified by column chromatography using 2:1 mixture solvent of hexane and ethyl acetate to obtain the title compound (170 mg, 85%).

¹H NMR (400 MHz, CDCl₃); δ 3.92 (1H, br s), 3.52˜3.34 (4H, m), 3.33 (2H, t), 2.04˜1.85 (2H, m), 1.46 (9H, s)

### Example 1-24

### 7-[2-(3-Methoxy-pyrrolidin-1-yl)-6-propyl-thieno[2,3-d]pyrimidin-4-yl]-3-trifluoromethyl-5,6,7,8-tetrahydro-[1,2,4]triazolo[4,3-a]pyrazine

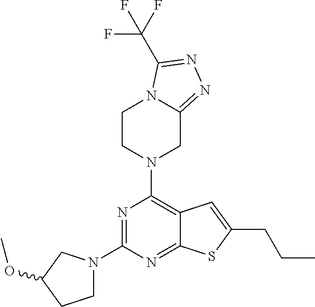

The compound obtained in Preparation Example 1-24-1 (40 mg, 0.2 mmol) was dissolved in 4.0 M hydrochloric acid dioxane solution and stirred for 1 hour. The mixture was distilled under reduced pressure to remove the solvent, and the compound obtained in Preparation Example 1-6 (40 mg, 0.1 mmol) and diisopropylethylamine (26 mg, 0.2 mmol) were added thereto and diluted with butanol (3 mL). The mixture was heated in a microwave reactor to 150° C. and stirred for 2 hours. The reaction solution was cooled to room temperature and distilled under reduced pressure, and purified by column chromatography using 1:1 mixture solvent of hexane and ethyl acetate to obtain the title compound (38 mg, 81%).

¹H NMR (400 MHz, CDCl₃); δ 6.76 (1H, s), 5.17 (2H, s), 4.34 (2H, t), 4.19 (2H, t), 4.05 (1H, m), 3/83.58 (4H, m), 3.37 (3H, s), 2.77 (2H, t), 2.19-2.00 (2H, m), 1.72 (2H, m), 0.99 (3H, t)

### Preparation Example 1-25-1

### 3-Oxo-piperazine-1-carboxylic acid tert-butyl ester

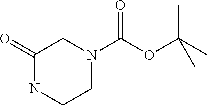

piperazin-2-one (500 mg, 5.0 mmol) was dissolved in methanol (15 mL), and di t-butyl dicarbonate (1.09 g, 5.0 mmol) was added thereto and the mixture was stirred for 16 hours. The mixture was distilled under reduced pressure to remove the solvent, and purified by column chromatography using 5:95 mixture solvent of methanol and dichloromethane to obtain the title compound (0.99 g, 99%).

¹H NMR (500 MHz, CDCl₃); δ 6.01 (1H, br s), 4.09 (2H, s), 3.63 (2H, t), 3.38 (2H, br s), 1.47 (9H, s)

### Preparation Example 1-25-2

### 4-Methyl-3-oxo-piperazine-1-carboxylic acid tert-butyl ester

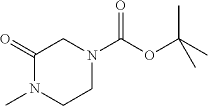

Except that the compound obtained in Preparation Example 1-25-1 (501 mg, 2.5 mmol) was used instead of pyrrolidin-3-ol, the same procedure as in Preparation Example 1-24-1 was carried out to obtain the title compound (462 mg, 86%).

¹H NMR (400 MHz, CDCl₃); δ 4.07 (2H, s), 3.65 (2H, t), 3.34 (2H, t), 3.00 (3H, s), 1.47 (9H, s)

### Example 1-25

### 1-Methyl-4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

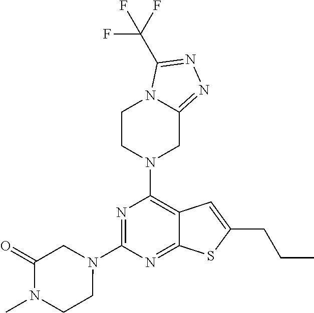

Except that the compound obtained in Preparation Example 1-25-2 (43 mg, 0.2 mmol) was used instead of the compound obtained in Preparation Example 1-24-1, the same procedure as in Example 1-24 was carried out to obtain the title compound (42 mg, 88%).

¹H NMR (400 MHz, CDCl₃); δ 6.83 (1H, s), 5.22 (2H, s), 4.39 (2H, s), 4.35 (2H, t), 4.23 (2H, t), 4.06 (2H, t), 3.44 (2H, t), 3.03 (3H, s), 2.79 (2H, t), 1.73 (2H, m), 1.00 (3H, t)

### Example 1-26

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-ol

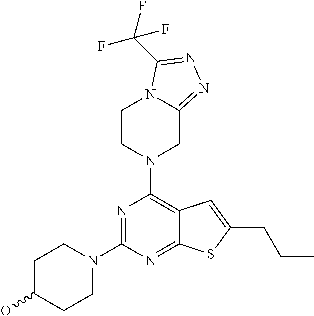

The compound obtained in Preparation Example 1-1-3 (40 mg, 0.1 mmol), piperidin-4-ol; hydrochloric acid salt (28 mg, 0.2 mmol) and diisopropylethylamine 26 mg (0.2 mmol) were diluted with butanol (3 mL), and the mixture was heated in a microwave reactor to 150° C. and stirred for 2 hours. The reaction solution was distilled under reduced pressure to remove the solvent, and purified by column chromatography using 7:93 mixture solvent of methanol and dichloromethane to obtain the title compound (37 mg, 79%).

¹H NMR (400 MHz, CDCl₃); δ 6.76 (1H, s), 5.16 (2H, s), 4.43˜4.30 (4H, m), 4.19 (2H, t), 3.94 (1H, m), 3.29 (2H, m), 2.77 (2H, t), 1.95 (2H, m), 1.72 (2H, m), 1.53 (2H, m), 0.99 (3H, t)

### Example 1-27

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ol

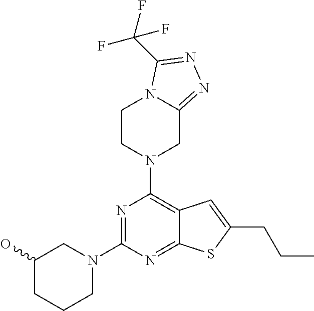

Except that piperidin-3-ol; hydrochloric acid salt (28 mg, 0.2 mmol) was used instead of piperidin-4-ol; hydrochloric acid salt, the same procedure as in Example 1-26 was carried out to obtain the title compound (43 mg, 91%).

¹H NMR (400 MHz, CDCl₃); δ 6.76 (1H, s), 5.15 (2H, m), 4.33 (2H, t), 4.27˜4.09 (3H, m), 3.93 (1H, m), 3.83 (1H, m), 3.55 (2H, m), 2.77 (2H, t), 2.26 (1H, br s), 1.95 (1H, m), 1.84 (1H, m), 1.71 (2H, m), 1.65 (1H, m), 1.54 (1H, m), 0.99 (3H, t)

### Example 1-28

### {1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-2-yl}-methanol

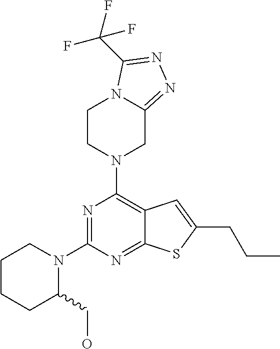

Except that piperidin-2-yl-methanol (23 mg, 0.2 mmol) was used instead of piperazin-2-one, the same procedure as in Example 1-1 was carried out to obtain the title compound (41 mg, 85%).

¹H NMR (400 MHz, CDCl₃); δ 6.75 (1H, s), 5.14 (2H, m), 4.97 (1H, m), 4.66 (1H, d), 4.34 (2H, m), 4.18 (2H, m), 3.92 (1H, dd), 3.72 (1H, dd), 3.03 (1H, t), 2.77 (br s), 2.76 (2H, t), 1.88˜1.44 (8H, m), 0.99 (3H, t)

### Example 1-29

### {1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-methanol

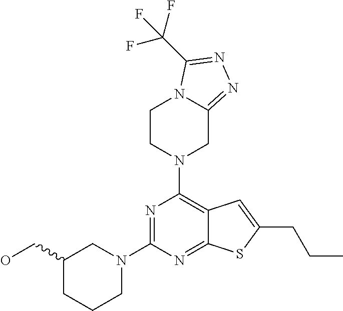

Except that piperidin-3-yl-methanol (23 mg, 0.2 mmol) was used instead of piperazin-2-one, the same procedure as in Example 1-1 was carried out to obtain the title compound (46 mg, 96%).

¹H NMR (400 MHz, CDCl₃); δ 6.76 (1H, s), 5.16 (2H, m), 4.33 (2H, t), 4.20 (2H, m), 4.10 (1H, d), 3.97 (1H, m), 3.68˜3.42 (4H, m), 2.76 (2H, t), 2.73 (1H, br s), 1.83 (1H, m), 1.78˜1.62 (4H, m), 1.50 (1H, m), 1.40 (1H, m), 0.99 (3H, t)

### Example 1-30

### {1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-2-yl}-methanol

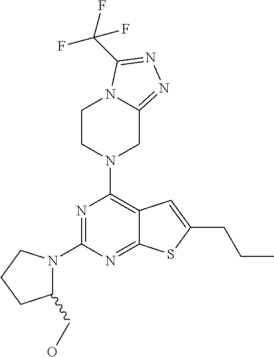

Except that pyrrolidin-2-yl-methanol (20 mg, 0.2 mmol) was used instead of piperazin-2-one, the same procedure as in Example 1-1 was carried out to obtain the title compound (46 mg, 98%).

¹H NMR (400 MHz, CDCl₃); δ 6.78 (1H, s), 5.19 (2H, s), 4.34 (2H, t), 4.33˜4.14 (3H, m), 3.78˜3.56 (4H, m), 2.77 (2H, t), 2.17˜1.63 (7H, m), 0.99 (3H, t)

### Example 1-31

### Cyclopentyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

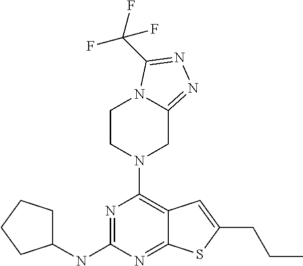

Except that cyclopentylamine (17 mg, 0.2 mmol) was used instead of piperazin-2-one, the same procedure as in Example 1-1 was carried out to obtain the title compound (38 mg, 84%).

¹H NMR (400 MHz, CDCl₃); δ 6.77 (1H, s), 5.18 (2H, s), 4.90 (1H, d), 4.34 (2H, t), 4.27 (1H, m), 4.19 (2H, t), 2.77 (2H, t), 2.04 (2H, m), 1.78˜1.58 (6H, m), 0.99 (3H, t)

### Example 1-32

### Benzyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

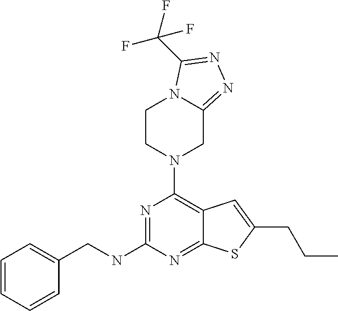

Except that benzylamine (21 mg, 0.2 mmol) was used instead of piperazin-2-one, the same procedure as in Example 1-1 was carried out to obtain the title compound (35 mg, 74%).

¹H NMR (500 MHz, CDCl₃); δ 7.36˜7.29 (4H, m), 7.24 (1H, t), 6.79 (1H, s), 5.40 (1H, br s), 5.16 (2H, s), 4.61 (2H, d), 4.10 (4H, s), 2.77 (2H, t), 1.71 (2H, m), 0.99 (3H, t)

### Preparation Example 1-33-1

### 2-((3aR,4S,6R,6aS)-6-Amino-2,2-dimethyl-tetrahydro-cyclopenta[1,3]dioxol-4-yloxy)-ethanol

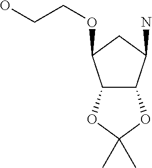

The synthesis was carried out according to a known method (WO 2001/092263).

### Preparation Example 1-33-2

### 2-{(3aR,4S,6R,6aS)-2,2-Dimethyl-6-[6-propyl-4-(3-trifluoromethyl-5,6-dihydro-8H-[1,2,4]triazolo[4,3-a]pyrazin-7-yl)-thieno[2,3-d]pyrimidin-2-ylmethyl]-tetrahydro-cyclopenta[1,3]dioxol-4-yloxy}-ethanol

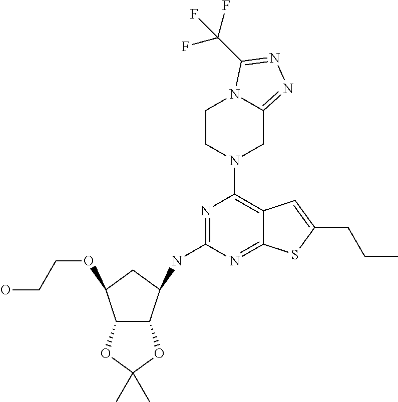

Except that the compound obtained in Preparation Example 1-33-1 (20 mg, 0.09 mmol) was used instead of piperazin-2-one, the same procedure as in Example 1-1 was carried out to obtain the title compound (7 mg, 13%).

¹H NMR (500 MHz, CDCl₃); δ 6.80 (1H, s), 5.34 (2H, s), 4.61 (1H, d), 4.51˜4.85 (5H, m), 3.95 (1H, d), 3.82˜3.39 (5H, m), 2.87 (1H, t), 2.76 (2H, t), 2.25 (1H, m), 1.94 (1H, d), 1.71 (2H, m), 1.45 (3H, s), 1.25 (3H, s), 1.00 (3H, t)

### Example 1-33

### (1S,2S,3S,5R)-3-(2-Hydroxy-ethoxy)-5-[6-propyl-4-(3-trifluoromethyl-5,6-dihydro-8H-[1,2,4]triazolo[4,3-a]pyrazin-7-yl)-thieno[2,3-d]pyrimidin-2-ylamino]-cyclopentane-1,2-diol

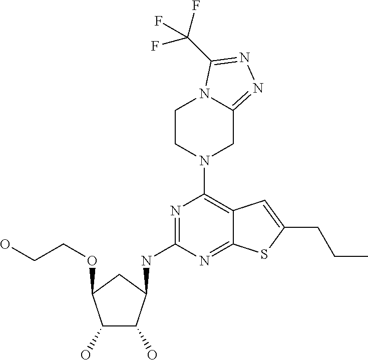

The compound obtained in Preparation Example 1-33-2 (7 mg, 0.012 mmol) was dissolved in 4.0 M hydrochloric acid dioxane solution (3 mL) and stirred for 1 hour. The mixture was distilled under reduced pressure to remove the solvent, and purified by column chromatography using ethyl acetate to obtain the title compound (2.3 mg, 35%).

¹H NMR (500 MHz, CDCl₃); δ 6.82 (1H, s), 5.20 (2H, m), 4.32 (2H, br s), 4.23 (2H, br s), 4.09 (2H, br s), 4.03 (1H, t), 3.91 (1H, m), 3.75 (2H, t), 3.69 (1H, m), 3.63 (1H, m), 2.78 (2H, t), 2.65 (1H, m), 2.03 (1H, d), 1.71 (2H, m), 0.99 (3H, t)

### Example 1-34

### 2-{(2-Hydroxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethanol

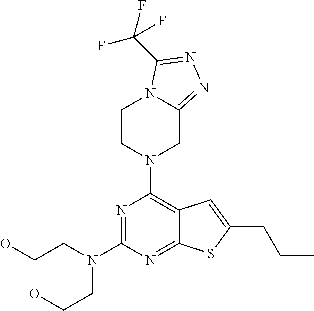

Except that 2-(2-hydroxy-ethylamino)-ethanol (78 mg, 0.74 mmol) was used instead of piperazin-2-one, the same procedure as in Example 1-1 was carried out to obtain the title compound (107 mg, 91%).

¹H NMR (500 MHz, CDCl₃); δ 6.78 (1H, s), 5.15 (2H, s), 4.31 (2H, t), 4.20 (2H, t), 3.88 (4H, t), 3.80 (4H, t), 3.50 (2H, br s), 2.77 (2H, t), 1.72 (2H, m), 0.99 (3H, t)

### Example 1-35

### 4-[6-Propyl-4-(3-trifluoromethyl-5,6-dihydro-8H-[1,2,4]triazolo[4,3-a]pyrazin-7-yl)-thieno[2,3-d]pyrimidin-2-yl]-morpholin-2-one

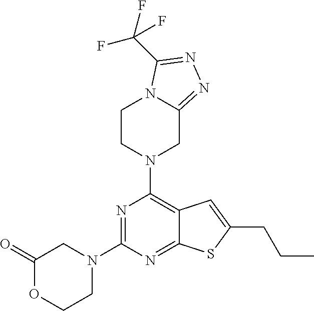

The compound obtained in Example 1-34 (17 mg, 0.036 mmol) was dissolved in dichloromethane (3 mL) and TPAP (1.3 mg, 0.004 mmol) and 4-methyl morpholin N-oxide (17 mg, 0.15 mmol) were added thereto, and the mixture was stirred for 2 hours. The reaction solution was distilled under reduced pressure to remove the solvent, and purified by column chromatography using 2:3 mixture solvent of hexane and ethyl acetate to obtain the title compound (7.2 mg, 42%).

¹H NMR (500 MHz, CDCl₃); δ 6.83 (1H, s), 5.22 (2H, s), 4.57 (2H, s), 4.52 (2H, t), 4.35 (2H, t), 4.24 (2H, t), 4.00 (2H, t), 2.79 (2H, t), 1.73 (2H, m), 0.99 (3H, t)

### Example 1-36

### Phenyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

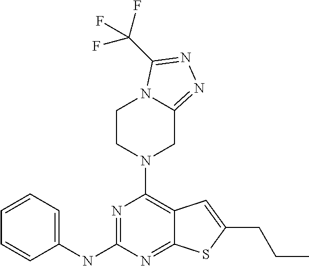

The compound obtained in Preparation Example 1-1-3 (50 mg, 0.124 mmol), aniline (23.6 mg, 0.248 mmol), palladium acetate (II) (2.79 mg, 0.012 mmol), BINAP (11.59 mg, 0.019 mmol) and cesium carbonate (61 mg, 0.186 mmol) were diluted with toluene (5 mL) and stirred for 2 hours under reflux. The reaction solution was cooled to room temperature, filtered by using celite, distilled under reduced pressure to remove the solvent, and purified by column chromatography using 1:10 mixture solvent of methanol and dichloromethane to obtain the title compound (37 mg, 65%).

¹H NMR (500N1 Hz, CDCl₃); δ 7.57 (2H, d), 7.34 (2H, t), 7.03 (1H, t), 6.91 (1H, s), 6.86 (1H, s), 5.24 (2H, s), 4.35 (2H, m), 4.24 (2H, m), 2.81 (2H, t), 1.75 (2H, m), 1.00 (3H, t)

### Example 1-37

### [6-Propyl-4-(3-trifluoromethyl-5,6-dihydro-8H-[1,2,4]triazolo[4,3-a]pyrazin-7-yl)-thieno[2,3-d]pyrimidin-2-yl]-pyrimidin-2-yl-amine

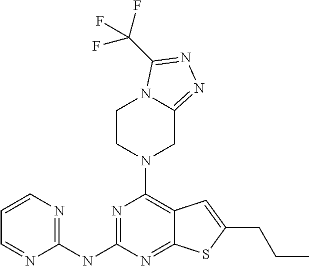

A procedure similar with Example 1-36 was carried out by using the compound obtained in Preparation Example 1-1-3 (50 mg, 0.124 mmol) and pyrimidin-2-ylamine (23.6 mg, 0.248 mmol) to obtain the title compound (13 mg, 23%).

¹H NMR (400 MHz, CDCl₃); δ 8.55 (2H, d), 7.83 (1H, br s), 6.94 (1H, s), 6.90 (1H, t), 5.31 (2H, s), 4.47 (2H, m), 4.31 (2H, m), 2.85 (2H, t), 1.75 (2H, q), 1.01 (3H, t)

### Example 1-38

### 4-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-phenol

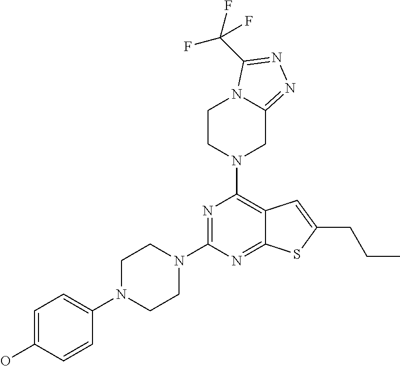

A procedure similar with Example 1-1 was carried out by using the compound obtained in Preparation Example 1-1-3 (40 mg, 0.099 mmol) and 4-piperazin-1-yl-phenol (53 mg, 0.298 mmol) to obtain the title compound (50 mg, 93%).

¹H NMR (400 MHz, CDCl₃); δ 6.88 (2H, d), 6.78 (2H, d), 5.18 (2H, s), 4.73 (1H, br), 4.35 (2H, t), 4.21 (2H, t), 3.94 (4H, m), 3.10 (4H, m), 2.78 (2H, t), 1.71 (2H, m), 0.99 (3H, t)

### Example 1-39

### 3-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-phenol

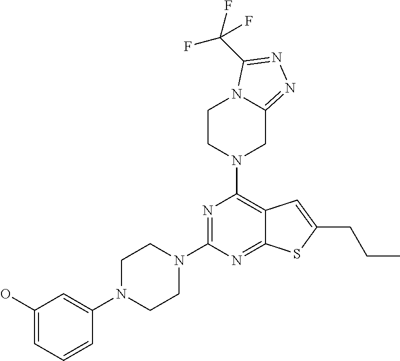

A procedure similar with Example 1-1 was carried out by using the compound obtained in Preparation Example 1-1-3 (40 mg, 0.099 mmol) and 3-piperazin-1-yl-phenol (53 mg, 0.298 mmol) to obtain the title compound (36 mg, 67%).

¹H NMR (400 MHz, CDCl₃); δ 7.14 (1H, t), 6.79 (1H, s), 6.54 (1H, m), 6.45 (1H, m), 6.36 (1H, m), 5.19 (2H, s), 5.01 (1H, s), 4.34 (2H, t), 4.22 (2H, t), 3.93 (4H, m), 3.22 (4H, m), 2.79 (2H, t), 1.72 (3H, m), 1.00 (3H, t)

### Preparation Example 1-40-1

### 4-Cyclopentyl-piperazine-1-carboxylic acid tert-butyl ester

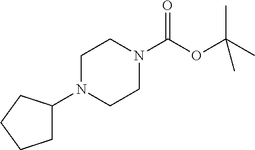

Piperazine-1-carboxylic acid tert-butyl ester (100 mg, 0.537 mmol) was dissolved in N,N-dimethylformamide (5 mL) and 60% sodium hydride (24 mg, 0.644 mmol) was added thereto. The mixture was stirred at 0° C. for 30 minutes and bromo-cyclopentane (96 mg, 0.591 mmol) was added thereto and stirred at 60° C. for 16 hours. The reaction solution was distilled under reduced pressure and purified by column chromatography using 1:5 mixture solvent of methanol and dichloromethane to obtain the title compound (40 mg, 29%).

¹H NMR (400 MHz, CDCl₃); δ 7.14 (1H, t), 6.79 (1H, s), 6.54 (1H, m), 6.45 (1H, m), 6.36 (1H, m), 5.19 (2H, s), 5.01 (1H, s), 4.34 (2H, t), 4.22 (2H, t), 3.93 (4H, m), 3.22 (4H, m), 2.79 (2H, t), 1.72 (3H, m), 1.00 (3H, t)

### Preparation Example 1-40-2

### 1-Cyclopentyl-piperazine; hydrochloride

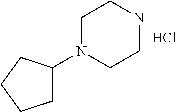

The compound obtained in Preparation Example 1-40-1 (40 mg, 0.157 mmol) was dissolved in dichloromethane (1 mL) and 4.0 M hydrochloric acid dioxane solution (2 mL) was added thereto, and then stirred at room temperature for 1 hour. The reaction solution was distilled under reduced pressure to obtain the title compound (35 mg, 98%).

Mass: M+H 155

### Example 1-40

### 7-[2-(4-Cyclopentyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine

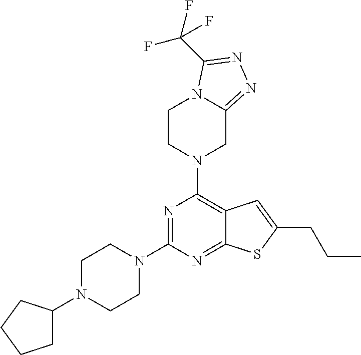

A procedure similar with Example 1-26 was carried out by using the compound obtained in Preparation Example 1-1-3 (66 mg, 0.163 mmol) and the compound obtained in Preparation Example 1-40-2 (37 mg, 0.163 mmol) to obtain the title compound (30 mg, 35%).

¹H NMR (400 MHz, CDCl₃); δ 6.75 (1H, s), 5.16 (2H, s), 4.34 (2H, m), 4.18 (2H, m), 3.81 (4H, m), 3.65 (1H, t), 2.77 (2H, t), 2.51 (4H, m), 1.98 (2H, m), 1.71 (4H, m), 1.56 (2H, m), 1.46 (2H, m), 1.01 (3H, t)

### Preparation Example 1-41-1

### 4-Cyclopentylmethyl-piperazine-1-carboxylic acid tert-butyl ester

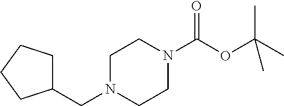

A procedure similar with Preparation Example 1-40-1 was carried out by using piperazine-1-carboxylic acid tert-butyl ester (150 mg, 0.805 mmol) and toluene-4-sulfonic acid cyclopentylmethyl ester (296 mg, 1.21 mmol) to obtain the title compound (100 mg, 46%).

¹H NMR (400 MHz, CDCl₃); δ 3.42 (4H, m), 2.36 (4H, m), 2.25 (2H, d), 2.05 (1H, m), 1.71-1.78 (2H, m), 1.49-1.61 (4H, m), 1.45 (9H, s), 1.18 (2H, m)

### Preparation Example 1-41-2

### 1-Cyclopentylmethyl-piperazine; hydrochloride

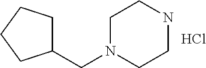

A procedure similar with Preparation Example 1-40-2 was carried out by using the compound obtained in Preparation Example 1-41-1 (100 mg, 0.373 mmol) to obtain the title compound (80 mg, 89%).

Mass: M+H 169

### Example 1-41

### 7-[2-(4-Cyclopentylmethyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine

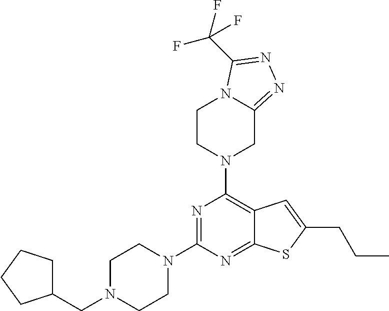

A procedure similar with Example 1-26 was carried out by using the compound obtained in Preparation Example 1-1-3 (50 mg, 0.124 mmol) and the compound obtained in Preparation Example 1-41-2 (59.9 mg, 0.248 mmol) to obtain the title compound (50 mg, 75%).

¹H NMR (400 MHz, CDCl₃); δ 6.76 (1H, s), 5.16 (2H, s), 4.34 (2H, m), 4.18 (2H, m), 3.79 (4H, m), 2.77 (2H, t), 2.48 (4H, m), 2.29 (2H, d), 2.11 (1H, m), 1.74 (2H, m), 1.71 (2H, m), 1.57 (3H, m), 1.23 (3H, m), 0.99 (3H, t)

### Preparation Example 1-42-1

### 4-Cyclohexylmethyl-piperazine-1-carboxylic acid tert-butyl ester

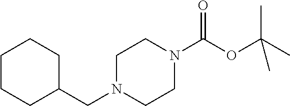

A procedure similar with Preparation Example 1-40-1 was carried out by using piperazine-1-carboxylic acid tert-butyl ester (150 mg, 0.805 mmol) and bromomethylhexane (143 mg, 0.805 mmol) to obtain the title compound (100 mg, 65%).

¹H NMR (500 MHz, CDCl₃); δ 3.39 (4H, m), 2.31 (4H, m), 2.09 (2H, d), 1.67˜1.75 (7H, m), 1.46 (9H, s), 1.16˜1.22 (2H, m), 0.85 (2H, m)

### Preparation Example 1-42-2

### 1-Cyclohexylmethyl-piperazine; hydrochloride

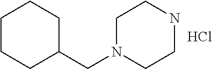

A procedure similar with Preparation Example 1-40-2 was carried out by using the compound obtained in Preparation Example 1-42-1 (100 mg, 0.354 mmol) to obtain the title compound (100 mg, 110%).

Mass: M+H 169

### Example 1-42

### 7-[2-(4-Cyclohexylmethyl-piperazin-1-yl)-6-propyl-thieno[2,3-d]pyrimidin-4-yl]-3-trifluoromethyl-5,6,7,8-tetrahydro-[1,2,4]triazolo[4,3-a]pyrazine

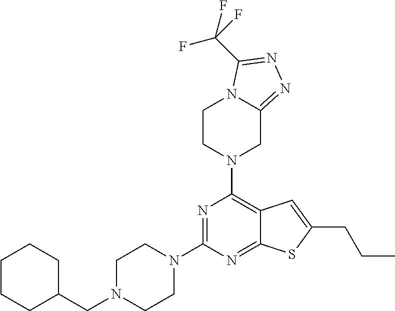

A procedure similar with Example 1-26 was carried out by using the compound obtained in Preparation Example 1-1-3 (50 mg, 0.124 mmol) and the compound obtained in Preparation Example 1-42-2 (63.4 mg, 0.248 mmol) to obtain the title compound (40 mg, 59%).

¹H NMR (400 MHz, CDCl₃); δ 6.76 (1H, s), 5.16 (2H, s), 4.34 (2H, m), 4.18 (2H, m), 3.79 (4H, m), 2.77 (2H, t), 2.44 (4H, m), 2.15 (2H, d), 1.78 (2H, m), 1.70 (6H, m), 1.55 (1H, m), 1.24 (2H, m), 1.01 (3H, t), 0.88 (2H, m)

### Example 1-43

### 3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid ethyl ester

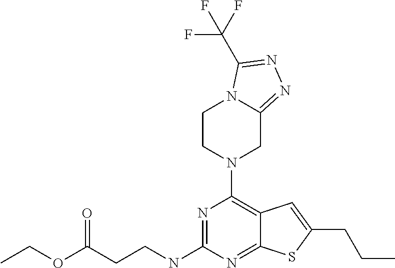

The compound obtained in Preparation Example 1-1-3 (24 mg, 0.06 mmol), 3-amino-propionic acid ethyl ester; hydrochloric acid salt (19 mg, 0.12 mmol), palladium acetate (II) (1.3 mg, 0.006 mmol), BINAP (4.5 mg, 0.007 mmol) and cesium carbonate (59 mg, 0.18 mmol) were diluted with toluene (5 mL) and stirred for 3 hours under reflux. The reaction solution was cooled to room temperature, filtered by using celite, distilled under reduced pressure to remove the solvent, and purified by column chromatography using 1:1 mixture solvent of hexane and ethyl acetate to obtain the title compound (7 mg, 24%).

¹H NMR (500 MHz, CDCl₃); δ 6.80 (1H, s), 5.22 (2H, s), 4.34 (2H, t), 4.24 (2H, t), 4.14 (2H, q), 3.73 (2H, q), 2.78 (2H, t), 2.62 (2H, t), 1.72 (2H, m), 1.25 (3H, t), 0.99 (3H, t)

### Example 1-44

### 3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid

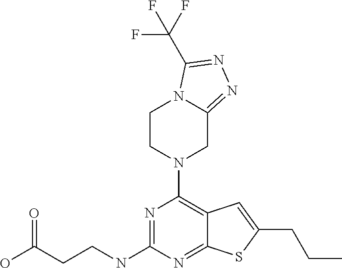

The compound obtained in Example 1-43 (3.8 mg, 0.008 mmol) was dissolved in tetrahydrofuran (3 mL) and methanol (0.5 mL) and 1.0 M sodium hydroxide solution (0.024 mL, 0.024 mmol) was added thereto. The mixture was stirred for 16 hours, acidified with 1.0 M hydrochloric acid solution, distilled under reduced pressure to remove the solvent, diluted with ethyl acetate, and washed with water and brine. The organic layer was dried with anhydrous magnesium sulfate, distilled under reduced pressure, and purified by column chromatography using 5:95 mixture solvent of methanol and dichloromethane to obtain the title compound (228 mg, 98%).

¹H NMR (500 MHz, 40 wt. % NaOD in D₂O); δ 7.04 (1H, s), 5.17 (2H, s), 4.40 (2H, t), 4.25 (2H, t), 3.64 (2H, t), 2.80 (2H, t), 2.59 (2H, t), 1.72 (2H, m), 0.99 (3H, t)

### Example 1-45

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid ethyl ester

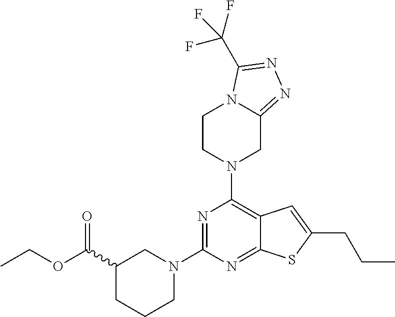

The compound obtained in Preparation Example 1-1-3 (403 mg, 1.0 mmol), piperidine-3-carboxylic acid ethyl ester (314 mg, 2.0 mmol), palladium acetate (II) (22 mg, 0.1 mmol), BINAP (75 mg, 0.12 mmol) and cesium carbonate (489 mg, 1.5 mmol) were diluted with toluene (10 mL) and stirred for 3 hours under reflux. The reaction solution was cooled to room temperature, filtered by using celite, distilled under reduced pressure to remove the solvent, and purified by column chromatography using 3:2 mixture solvent of hexane and ethyl acetate to obtain the title compound (246 mg, 47%).

¹H NMR (400 MHz, CDCl₃); δ 6.77 (1H, s), 5.17 (2H, m), 4.71 (1H, dd), 4.49 (1H, d), 4.36 (2H, t), 4.28˜4.12 (4H, m), 3.22 (1H, dd), 3.05 (1H, t), 2.77 (2H, t), 2.51 (1H, m), 2.09 (1H, m), 1.87˜1.66 (41-1H, m), 1.53 (1H, m), 1.28 (3H, t), 0.99 (3H, t)

### Example 1-46

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid

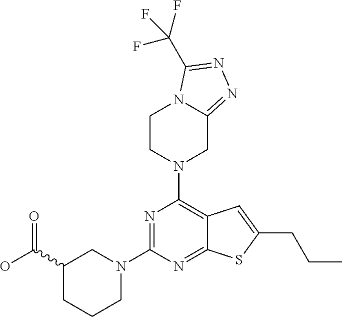

The compound obtained in Example 1-45 (246 mg, 0.47 mmol) was dissolved in tetrahydrofuran (5 mL) and methanol (1 mL) and 1.0 M sodium hydroxide solution (1.4 mL, 1.4 mmol) was added thereto. The mixture was stirred for 16 hours, acidified with 6.0 M hydrochloric acid solution, distilled under reduced pressure to remove the solvent, diluted with ethyl acetate, and washed with water and brine. The organic layer was dried with anhydrous magnesium sulfate, distilled under reduced pressure, and purified by column chromatography using 5:95 mixture solvent of methanol and dichloromethane to obtain the title compound (228 mg, 98%).

¹H NMR (400 MHz, CDCl₃); δ 6.78 (1H, s), 5.19 (2H, s), 4.70 (1H, d), 4.45 (1H, d), 4.36 (2H, t), 4.20 (2H, m), 3.30 (1H, dd), 3.11 (1H, t), 2.77 (2H, t), 2.57 (1H, m), 2.10 (1H, m), 1.88-1.68 (4H, m), 1.54 (1H, m), 1.28 (3H, t), 0.99 (3H, t)

### Example 1-47

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid isopropyl ester

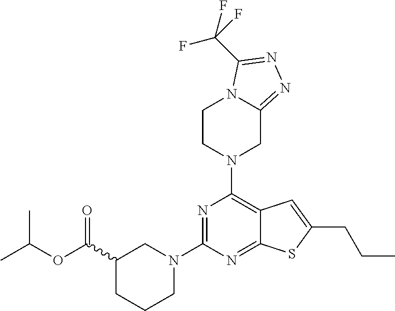

The compound obtained in Example 1-46 (50 mg, 0.1 mmol) was dissolved in N,N-dimethylformamide (5 mL), and potassium carbonate (28 mg, 0.2 mmol) and 2-iodopropane (21 mg, 0.15 mmol) were added thereto. The mixture was stirred at 60° C. for 3 hours, cooled to room temperature, distilled under reduced pressure to remove the solvent, diluted with ethyl acetate, and washed with water and brine. The organic layer was dried with anhydrous magnesium sulfate, distilled under reduced pressure, and purified by column chromatography using 3:2 mixture solvent of hexane and ethyl acetate to obtain the title compound (40 mg, 74%).

¹H NMR (400 MHz, CDCl₃); δ 6.77 (1H, s), 5.17 (2H, m), 5.03 (1H, m), 4.68 (1H, dd), 4.47 (1H, d), 4.36 (2H, t), 4.27-4.08 (2H, m), 3.24 (1H, dd), 3.07 (1H, m), 2.77 (2H, t), 2.46 (1H, m), 2.07 (1H, m), 1.83-1.67 (4H, m), 1.53 (1H, m), 1.25 (3H, d) 1.23 (2H, d), 0.99 (3H, t)

### Example 1-48

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid 2,2-dimethyl-propionyloxymethyl ester

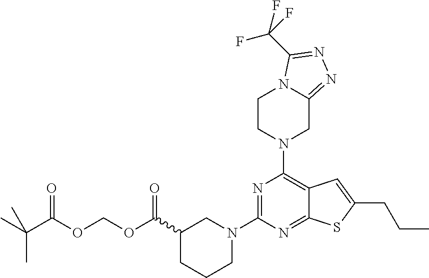

The compound obtained in Example 1-46 (50 mg, 0.1 mmol) was dissolved in N,N-dimethylformamide (5 mL), and potassium carbonate (28 mg, 0.2 mmol) and chloromethyl pyvalate (23 mg, 0.15 mmol) were added thereto. The mixture was stirred at 60° C. for 3 hours, cooled to room temperature, distilled under reduced pressure to remove the solvent, diluted with ethyl acetate, and washed with water and brine. The organic layer was dried with anhydrous magnesium sulfate, distilled under reduced pressure, and purified by column chromatography using 3:2 mixture solvent of hexane and ethyl acetate to obtain the title compound (49 mg, 80%).

¹H NMR (400 MHz, CDCl₃); δ 6.79 (1H, s), 5.77 (2H, s), 5.19 (2H, m), 4.63 (1H, dd), 4.43 (1H, t), 4.39 (2H, t), 4.28˜4.14 (2H, m), 3.32 (1H, dd), 3.14 (1H, t), 2.77 (2H, t), 2.58 (1H, m), 2.05 (1H, m), 1.87˜1.66 (4H, m), 1.54 (1H, m), 1.19 (9H, s), 0.99 (3H, t)

### Example 1-49

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid methyl ester

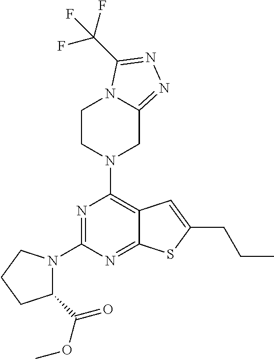

Except that (S)-pyrrolidine-2-carboxylic acid methyl ester; hydrochloric acid salt (500 mg, 3.0 mmol) was used instead of piperidin-3-carboxylic acid ethyl ester, the same procedure as in Example 1-45 was carried out to obtain the title compound (310 mg, 42%).

¹H NMR (400 MHz, CDCl₃); δ 6.78 (1H, s), 5.18 (2H, s), 4.53 (1H, br s), 4.34 (1H, m), 4.28 (1H, br s), 4.14 (2H, br s), 3.82 (1H, m), 3.72 (1H, m), 3.70 (3H, s), 2.77 (2H, t), 2.34 (1H, m), 2.19˜1.95 (3H, m), 1.72 (2H, m), 0.99 (3H, t)

### Example 1-50

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid

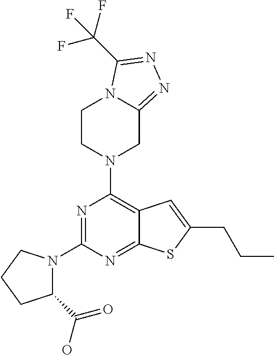

Except that the compound obtained in Example 1-49 (310 mg, 0.626 mmol) was used instead of the compound obtained in Example 1-45, the same procedure as in Example 1-46 was carried out to obtain the title compound (255 mg, 85%).

¹H NMR (400 MHz, CDCl₃); δ 6.81 (1H, s), 5.20 (2H, m), 4.50 (1H, br s), 4.32 (2H, br s), 4.22 (2H, br s), 3.66 (2H, d), 2.77 (2H, t), 2.46 (1H, br s), 2.19-1.94 (3H, m), 1.71 (2H, m), 1.00 (3H, t)

### Example 1-51

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid isopropyl ester

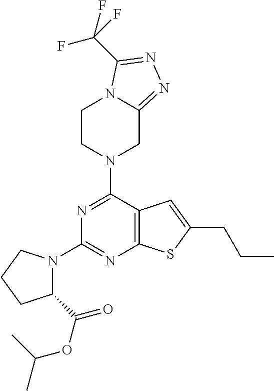

Except that the compound obtained in Example 1-50 (48 mg, 0.1 mmol) was used instead of the compound obtained in Example 1-46, the same procedure as in Example 1-47 was carried out to obtain the title compound (48 mg, 92%).

¹H NMR (400 MHz, CDCl₃); δ 6.78 (1H, s), 5.18 (2H, s), 5.00 (1H, m), 4.49 (1H, dd), 4.42-4.02 (4H, m), 3.81 (1H, m), 3.72 (1H, br s), 2.77 (2H, t), 2.32 (1H, m), 2.18-1.94 (3H, m), 1.71 (2H, m), 1.33˜1.05 (6H, m), 0.99 (3H, t)

### Example 1-52

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid 2,2-dimethyl-propionyloxymethyl ester

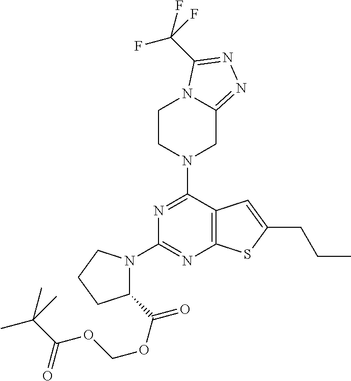

Except that the compound obtained in Example 1-50 (48 mg, 0.1 mmol) was used instead of the compound obtained in Example 1-46, the same procedure as in Example 1-48 was carried out to obtain the title compound (57 mg, 95%).

¹H NMR (400 MHz, CDCl₃); δ 6.81 (1H, br s), 5.84 (1H, d), 5.64 (1H, br s), 5.21 (2H, s), 4.55 (1H, br s), 4.35 (2H, t), 4.15 (2H, br s), 3.87-3.60 (2H, m), 2.77 (2H, t), 2.36 (1H, m), 2.18˜1.96 (3H, m), 1.71 (2H, m), 1.05 (9H, br s), 0.99 (3H, t)

### Example 1-53

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid methyl ester

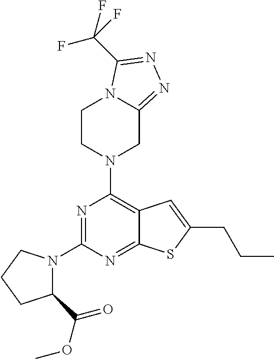

Except that (R)-pyrrolidine-2-carboxylic acid methyl ester; hydrochloric acid salt (33 mg, 0.2 mmol) was used instead of piperidin-3-carboxylic acid ethyl ester, the same procedure as in Example 1-45 was carried out to obtain the title compound (25 mg, 50%).

¹H NMR (400 MHz, CDCl₃); δ 6.78 (1H, s), 5.18 (2H, s), 4.53 (1H, br s), 4.34 (1H, m), 4.28 (1H, br s), 4.14 (2H, br s), 3.82 (1H, m), 3.72 (1H, m), 3.70 (3H, s), 2.77 (2H, t), (1H, m), 2.19˜1.95 (3H, m), 1.72 (2H, m), 0.99 (3H, t)

### Example 1-54

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-2-carboxylic acid

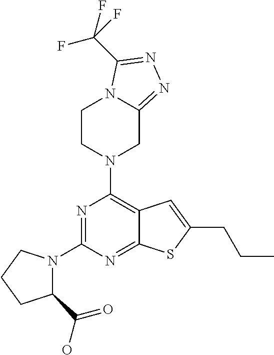

Except that the compound obtained in Example 1-53 (25 mg, 0.05 mmol) was used instead of the compound obtained in Example 1-45, the same procedure as in Example 1-46 was carried out to obtain the title compound (24 mg, 70%).

¹H NMR (400 MHz, CDCl₃); δ 6.81 (1H, s), 5.20 (2H, m), 4.50 (1H, br s), 4.32 (2H, br s), 4.22 (2H, br s), 3.66 (2H, d), 2.77 (2H, t), 2.46 (1H, br s), 2.19˜1.94 (3H, m), 1.71 (2H, m), 1.00 (3H, t)

### Preparation Example 1-55-1

### 3-Methanesulfonyloxy-pyrrolidine-1-carboxylic acid tert-butyl ester

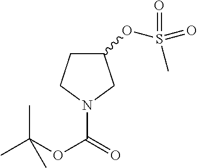

3-Hydroxypyrrolidine-1-carboxylic acid tert-butyl ester (0.94 g, 5.0 mmol) was dissolved in dichloromethane (10 mL) and cooled to 0° C., and then diisopropylethylamine (0.97 g, 7.5 mmol) and methanesulfonylchloride (0.63 g, 5.5 mmol) were added thereto. The mixture was stirred at room temperature for 16 hours, distilled under reduced pressure to remove the solvent, diluted with ethyl acetate, and washed with water and brine. The organic layer was dried with anhydrous magnesium sulfate, distilled under reduced pressure, and purified by column chromatography using 2:1 mixture solvent of hexane and ethyl acetate to obtain the title compound (1.27 g, 95%).

### Preparation Example 1-55-2

### 3-Cyano-pyrrolidine-1-carboxylic acid tert-butyl ester

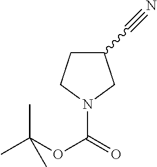

The compound obtained in Preparation Example 1-55-1 (1.27 g, 4.79 mmol) was dissolved in N,N-dimethylformamide (15 mL), and lithium cyanide (0.47 g, 14.37 mmol) was added thereto. The mixture was stirred at 80° C. for 16 hours, cooled to room temperature, distilled under reduced pressure to remove the solvent, diluted with ethyl acetate, and washed with water and brine. The organic layer was dried with anhydrous magnesium sulfate, distilled under reduced pressure, and purified by column chromatography using 2:1 mixture solvent of hexane and ethyl acetate to obtain the title compound (0.66 g, 70%).

¹H NMR (400 MHz, CDCl₃); δ 3.67 (1H, br s), 3.58 (2H, br s), 3.45 (1H, br s), 3.09 (1H, m), 2.25 (2H, m), 1.47 (9H, s)

### Example 1-55

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carbonitrile

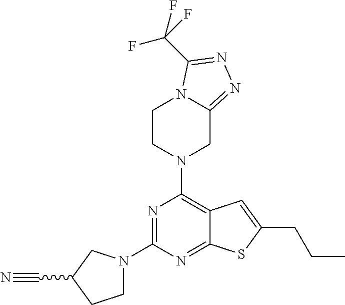

Except that the compound obtained in Preparation Example 1-55-2 (39 mg, 0.2 mmol) was used instead of the compound obtained in Preparation Example 1-24-1, the same procedure as in Example 1-24 was carried out to obtain the title compound (14 mg, 30%).

¹H NMR (400 MHz, CDCl₃); δ 6.80 (1H, s), 5.19 (2H, s), 4.35 (2H, t), 4.22 (2H, t), 3.90 (2H, m), 3.82 (1H, m), 3.69 (1H, m), 3.23 (1H, m), 2.79 (2H, t), 2.37 (2H, m), 1.73 (2H, m), 1.00 (3H, t)

### Preparation Example 1-56-1

### (R)-3-Methanesulfonyloxy-pyrrolidine-1-carboxylic acid tert-butyl ester

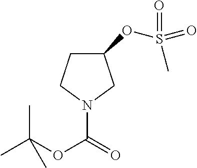

Except that (R)-3-hydroxypyrrolidine-1-carboxylic acid tert-butyl ester (7.78 g, 41.55 mmol) was used instead of 3-hydroxypyrrolidine-1-carboxylic acid tert-butyl ester, the same procedure as in Preparation Example 1-55-1 was carried out to obtain the title compound (10.88 g, 99%).

¹H NMR (400 MHz, CDCl₃); δ 5.26 (1H, m), 3.76˜3.40 (4H, m), 3.05 (3H, s), 2.28 (1H, m), 2.14 (1H, m), 1.47 (9H, s)

### Preparation Example 1-56-2

### (S)-3-Cyano-pyrrolidine-1-carboxylic acid tert-butyl ester

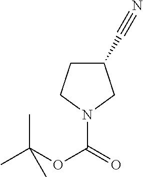

Except that the compound obtained in Preparation Example 1-56-1 (10.88 g, 41.0 mmol) was used instead of the compound obtained in Preparation Example 1-55-1, the same procedure as in Preparation Example 1-55-2 was carried out to obtain the title compound (10.88 g, 99%).

¹H NMR (400 MHz, CDCl₃); δ 3.67 (1H, br s), 3.58 (2H, br s), 3.45 (1H, br s), 3.09 (1H, m), 2.25 (2H, m), 1.47 (9H, s)

### Preparation Example 1-56-3

### (S)-Pyrrolidine-1,3-dicarboxylic acid 1-tert-butyl ester 3-methyl ester

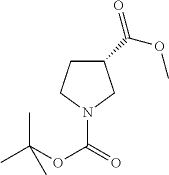

The compound obtained in Preparation Example 1-56-2 (0.66 g, 3.35 mmol) was mixed with aqueous solution of concentrated hydrochloric acid (5 mL), and the mixture was stirred at 100° C. for 3 hours. The mixture was cooled to room temperature, distilled under reduced pressure to remove the solvent, dissolved in methanol (10 mL), and cooled to 0° C. Chlorotrimethylsilane (1.45 g, 13.39 mmol) was added thereto, and the mixture was stirred at room temperature for 16 hours, cooled to 0° C. again, and diisopropylethylamine (2.59 g, 20.09 mmol) and di-t-butyl dicarbonate (0.8 g, 3.68 mmol) were added thereto. The mixture was stirred at room temperature for 16 hours, distilled under reduced pressure to remove the solvent, diluted with ethyl acetate, and washed with water and brine. The organic layer was dried with anhydrous magnesium sulfate, distilled under reduced pressure, and purified by column chromatography using 3:1 mixture solvent of hexane and ethyl acetate to obtain the title compound (0.66 g, 70%).

¹H NMR (500 MHz, CDCl₃); δ 3.71 (3H, s), 3.67˜3.40 (3H, m), 3.34 (1H, m), 3.04 (1H, m), 2.12 (2H, br s), 1.45 (9H, s)

### Example 1-56

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methyl ester

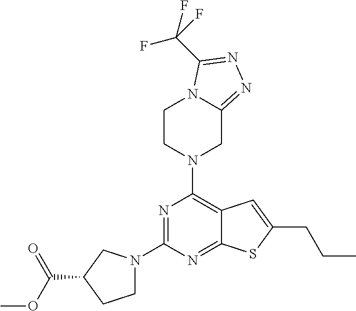

The compound obtained in Preparation Example 1-56-3 (229 mg, 1.0 mmol) was dissolved in 4.0 M hydrochloric acid dioxane solution (5 mL). The mixture was stirred for 1 hour and distilled under reduced pressure to remove the solvent. The compound obtained in Preparation Example 1-1-3 (200 mg, 0.5 mmol), palladium acetate (II) (11 mg, 0.05 mmol), BINAP (24 mg, 0.06 mmol) and cesium carbonate (489 mg, 1.5 mmol) were added thereto, diluted with toluene (5 mL) and stirred for 3 hours under reflux. The reaction solution was cooled to room temperature, filtered by using celite, distilled under reduced pressure to remove the solvent, and purified by column chromatography using 1:1 mixture solvent of hexane and ethyl acetate to obtain the title compound (154 mg, 62%).

¹H NMR (500 MHz, CDCl₃); δ 6.76 (1H, s), 5.16 (2H, s), 4.33 (2H, t), 4.19 (2H, t), 3.87 (1H, dd), 3.80˜3.63 (5H, m), 3.58 (1H, m), 3.17 (1H, m), 2.76 (2H, t), 2.25 (2H, m), 1.70 (2H, m), 0.98 (3H, t)

### Example 1-57

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid

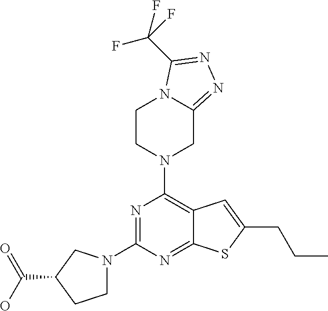

Except that the compound obtained in Example 1-56 (154 mg, 0.31 mmol) was used instead of the compound obtained in Example 1-45, the same procedure as in Example 1-46 was carried out to obtain the title compound (143 mg, 95%).

¹H NMR (400 MHz, CDCl₃+CD₃OD); δ 6.74 (1H, s), 5.12 (2H, s), 4.30 (2H, t), 4.16 (2H, t), 3.84˜3.64 (3H, m), 3.54 (1H, m), 3.12 (1H, m), 2.72 (2H, t), 2.21 (2H, m), 1.66 (2H, m), 0.94 (3H, t)

### Example 1-58

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid isopropyl ester

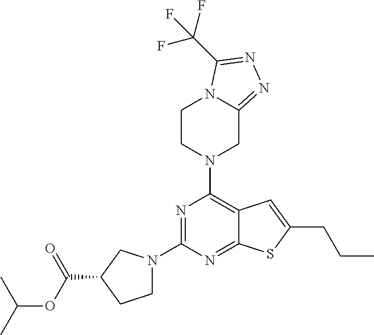

Except that the compound obtained in Example 1-57 (50 mg, 0.1 mmol) was used instead of the compound obtained in Example 1-46, the same procedure as in Example 1-47 was carried out to obtain the title compound (40 mg, 77%).

¹H NMR (400 MHz, CDCl₃); δ 6.77 (1H, s), 5.17 (2H, s), 5.04 (1H, m), 4.35 (2H, t), 4.20 (2H, t), 3.88 (1H, dd), 3.76 (2H, m), 3.59 (1H, m), 3.13 (1H, m), 2.77 (2H, t), 2.25 (2H, m), 1.72 (2H, m), 1.26 (6H, d), 0.99 (3H, t)

### Example 1-59

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid 2,2-dimethyl-propionyloxymethyl ester

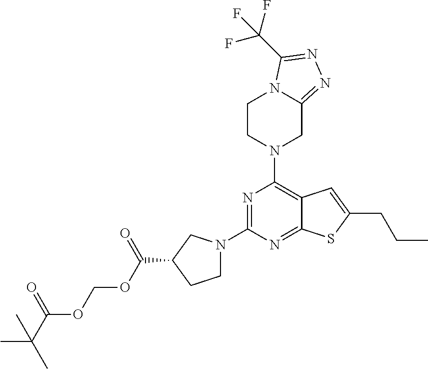

Except that the compound obtained in Example 1-57 (50 mg, 0.1 mmol) was used instead of the compound obtained in Example 1-46, the same procedure as in Example 1-48 was carried out to obtain the title compound (51 mg, 85%).

¹H NMR (400 MHz, CDCl₃); δ 6.78 (1H, s), 5.79 (2H, q), 5.18 (2H, s), 4.35 (2H, t), 4.21 (2H, t), 3.86 (2H, m), 3.74 (1H, m), 3.61 (1H, m), 3.22 (1H, m), 2.77 (2H, t), 2.27 (2H, m), 1.72 (2H, m), 1.20 (9H, s), 0.99 (3H, t)

### Preparation Example 1-60-1

### (S)-3-Methanesulfonyloxy-pyrrolidine-1-carboxylic acid tert-butyl ester

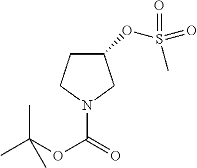

Except that (S)-3-hydroxypyrrolidine-1-carboxylic acid tert-butyl ester (13.46 g, 73 mmol) was used instead of 3-hydroxypyrrolidine-1-carboxylic acid tert-butyl ester, the same procedure as in Preparation Example 1-55-1 was carried out to obtain the title compound (19.42 g, 100%).

¹H NMR (400 MHz, CDCl₃); δ 5.26 (1H, m), 3.76-3.40 (4H, m), 3.05 (3H, s), 2.28 (1H, m), 2.14 (1H, m), 1.47 (9H, s)

### Preparation Example 1-60-2

### (R)-3-Cyano-pyrrolidine-1-carboxylic acid tert-butyl ester

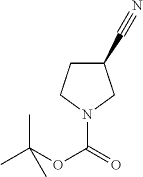

Except that the compound obtained in Preparation Example 1-60-1 (19.42 g, 73 mmol) was used instead of the compound obtained in Preparation Example 1-55-1, the same procedure as in Preparation Example 1-55-2 was carried out to obtain the title compound (9.64 g, 67%).

¹H NMR (400 MHz, CDCl₃); δ 3.67 (1H, br s), 3.58 (2H, br s), 3.45 (1H, br s), 3.09 (1H, m), 2.25 (2H, m), 1.47 (9H, s)

### Preparation Example 1-60-3

### (R)-Pyrrolidine-1,3-dicarboxylic acid 1-tert-butyl ester 3-methyl ester

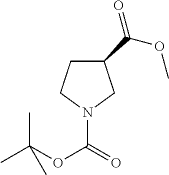

Except that the compound obtained in Preparation Example 1-60-2 (9.64 g, 49.12 mmol) was used instead of the compound obtained in Preparation Example 1-56-2, the same procedure as in Preparation Example 1-56-3 was carried out to obtain the title compound (9.98 g, 89%).

¹H NMR (500 MHz, CDCl₃); δ 3.71 (3H, s), 3.67˜3.40 (3H, m), 3.34 (1H, m), 3.04 (1H, m), 2.12 (2H, br s), 1.45 (9H, s)

### Example 1-60

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methyl ester

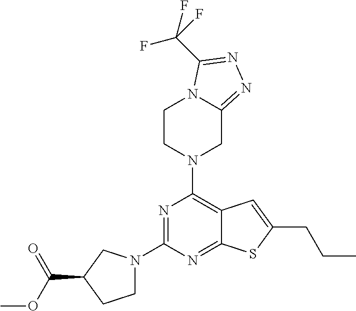

Except that the compound obtained in Preparation Example 1-60-3 (9.98 g, 43.53 mmol) was used instead of the compound obtained in Preparation Example 1-56-3, the same procedure as in Example 1-56 was carried out to obtain the title compound (11.14 g, 77%).

¹H NMR (500 MHz, CDCl₃); δ 6.76 (1H, s), 5.16 (2H, s), 4.33 (2H, t), 4.19 (2H, t), 3.87 (1H, dd), 3.80˜3.63 (5H, m), 3.58 (1H, m), 3.17 (1H, m), 2.76 (2H, t), 2.25 (2H, m), 1.70 (2H, m), 0.98 (3H, t)

### Example 1-61

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid

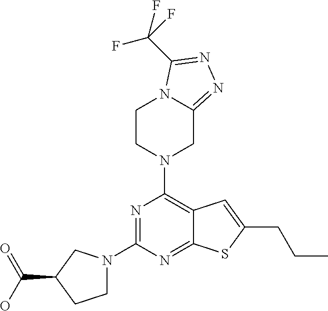

Except that the compound obtained in Example 1-60 (10.94 g, 22.08 mmol) was used instead of the compound obtained in Example 1-45, the same procedure as in Example 1-46 was carried out to obtain the title compound (9.7 g, 91%).

¹H NMR (400 MHz, CDCl₃+CD₃OD); δ 6.74 (1H, s), 5.12 (2H, s), 4.30 (2H, t), 4.16 (2H, t), 3.84˜3.64 (3H, m), 3.54 (1H, m), 3.12 (1H, m), 2.72 (2H, t), 2.21 (2H, m), 1.66 (2H, m), 0.94 (3H, t)

### Example 1-62

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid isopropyl ester

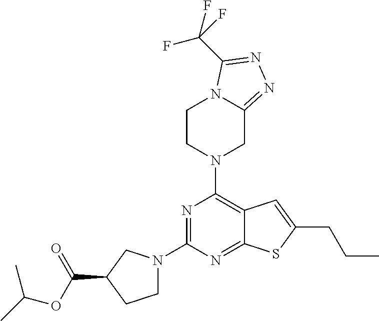

Except that the compound obtained in Example 1-61 (200 mg, 0.415 mmol) was used instead of the compound obtained in Example 1-46, the same procedure as in Example 1-47 was carried out to obtain the title compound (216 mg, 99%).

¹H NMR (400 MHz, CDCl₃); δ 6.77 (1H, s), 5.17 (2H, s), 5.04 (1H, m), 4.35 (2H, t), 4.20 (2H, t), 3.88 (1H, dd), 3.76 (2H, m), 3.59 (1H, m), 3.13 (1H, m), 2.77 (2H, t), 2.25 (2H, m), 1.72 (2H, m), 1.26 (6H, d), 0.99 (3H, t)

### Example 1-63

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid 2,2-dimethyl-propionyloxymethyl ester

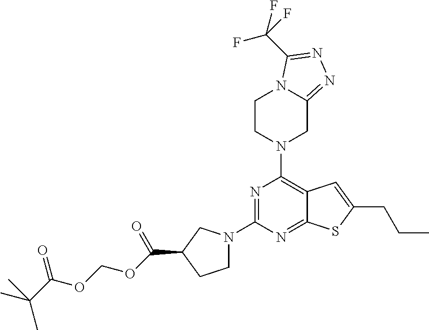

Except that the compound obtained in Example 1-61 (200 mg, 0.415 mmol) was used instead of the compound obtained in Example 1-46, the same procedure as in Example 1-48 was carried out to obtain the title compound (228 mg, 92%).

¹H NMR (400 MHz, CDCl₃); δ 6.78 (1H, s), 5.79 (2H, q), 5.18 (2H, s), 4.35 (2H, t), 4.21 (2H, t), 3.86 (2H, m), 3.74 (1H, m), 3.61 (1H, m), 3.22 (1H, m), 2.77 (2H, t), 2.27 (2H, m), 1.72 (2H, m), 1.20 (9H, s), 0.99 (3H, t)

### Example 1-64

### Dimethyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

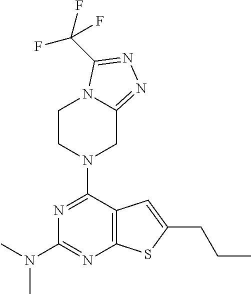

Except that dimethylamine; hydrochloric acid salt (16 mg, 0.2 mmol) was used instead of piperidin-4-ol; hydrochloric acid salt, the same procedure as in Example 1-26 was carried out to obtain the title compound (39 mg, 95%).

¹H NMR (400 MHz, CDCl₃); δ 6.76 (1H, s), 5.16 (2H, s), 4.35 (2H, t), 4.19 (2H, t), 3.17 (6H, s), 2.77 (2H, t), 1.72 (2H, m), 0.99 (3H, t)

### Example 1-65

### 2-[6-Propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline

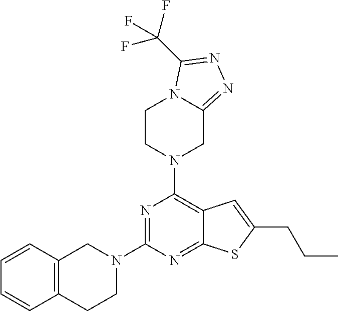

Except that 1,2,3,4-tetrahydroisoquinoline (27 mg, 0.2 mmol) was used instead of piperazin-2-one, the same procedure as in Example 1-1 was carried out to obtain the title compound (46 mg, 92%).

¹H NMR (400 MHz, CDCl₃); δ 7.25˜7.14 (4H, m), 6.77 (1H, s), 5.19 (2H, s), 4.91 (2H, s), 4.36 (2H, t), 4.21 (2H, t), 4.05 (2H, t), 2.92 (2H, t), 2.78 (2H, t), 1.72 (2H, m), 1.00 (3H, t)

### Example 1-66

### 6,7-Dimethoxy-2-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline

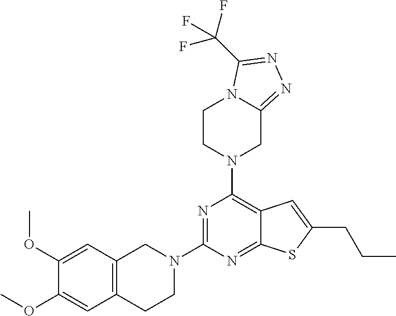

Except that 6,7-dimethoxy-1,2,3,4-tetrahydroisoquinoline (39 mg, 0.2 mmol) was used instead of piperazin-2-one, the same procedure as in Example 1-1 was carried out to obtain the title compound (55 mg, 98%).

¹H NMR (400 MHz, CDCl₃); δ 6.76 (1H, s), 6.73 (1H, s), 6.65 (1H, s), 5.19 (2H, s), 4.84 (2H, s), 4.37 (2H, t), 4.21 (2H, t), 4.04 (2H, t), 3.90 (3H, s), 3.86 (3H, s), 2.84 (2H, t), 2.78 (2H, t), 1.72 (2H, m), 1.00 (3H, t)

### Example 1-67

### 1-Ethyl-6,7-dimethoxy-2-[6-propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-isoquinoline

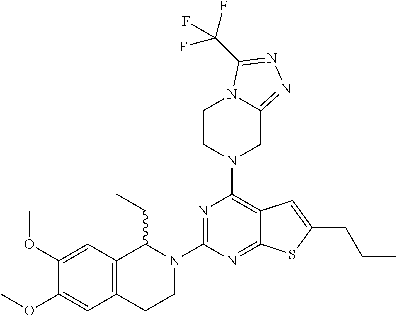

Except that 1-ethyl-6,7-dimethoxy-1,2,3,4-tetrahydroisoquinoline (44 mg, 0.2 mmol) was used instead of piperazin-2-one, the same procedure as in Example 1-1 was carried out to obtain the title compound (41 mg, 69%).

¹H NMR (400 MHz, CDCl₃); δ 6.73 (1H, s), 6.70 (1H, s), 6.60 (1H, s), 5.61 (1H, br s), 5.14 (2H, m), 4.61 (1H, br s), 4.38 (1H, m), 4.35 (2H, t), 4.16 (2H, m), 3.90 (3H, s), 3.84 (3H, s), 3.46 (1H, m), 2.88 (1H, t), 2.77 (2H, t), 2.71 (1H, m), 1.90 (2H, m), 1.72 (2H, q), 1.06-0.92 (6H, m)

### Preparation Example 1-68-1

### 1,2,3,4-Tetrahydroquinoxaline

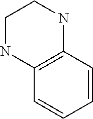

The synthesis was carried out according to a known method (J. Heterocyclic Chem., 42, 1031 (2005)).

### Example 1-68

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1,2,3,4-tetrahydro-quinoxaline

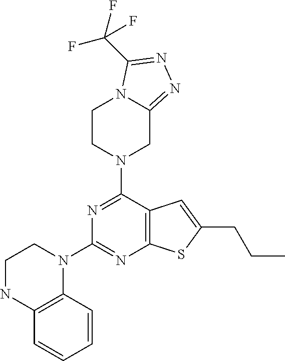

14 mg (28%) of the title compound was obtained according to the same method as Example 1-1 except that 27 mg (0.2 mmol) of the compound obtained from Preparation Example 1-68-1 was used instead of piperazine-2-one.

¹H NMR (400 MHz, CDCl₃); δ 6.62 (1H, d), 6.92 (1H, t), 6.83 (1H, s), 6.69 (1H, t), 6.61 (1H, d), 5.18 (2H, s), 4.24 (2H, t), 4.16 (4H, m), 4.02 (1H, br s), 3.45 (2H, t), 2.80 (2H, t), 1.73 (2H, m), 1.01 (3H, t)

### Example 1-69

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-ylamine

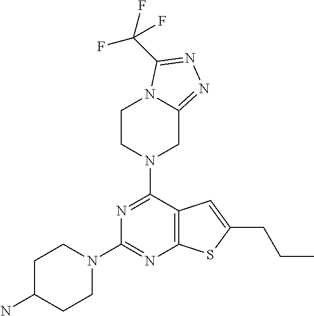

31 mg (66%) of the title compound was obtained according to the same method as Example 1-1 except that instead of piperazine-2-one, 40 mg (0.2 mmol) of piperidine-4-yl-carboxylic acid tert-butyl ester was used and treated with 1N HCl after reaction.

¹H NMR (400 MHz, CDCl₃+CD₃OD); δ 6.79 (1H, s), 5.16 (2H, s), 4.71 (2H, d), 4.35 (2H, t), 4.21 (2H, t), 3.07 (1H, m), 2.94 (2H, t), 2.79 (2H, t), 1.98 (2H, m), 1.72 (2H, m), 1.41 (2H, m), 1.00 (3H, t)

### Preparation Example 1-70-1

### {1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-carbamic acid tert-butyl ester

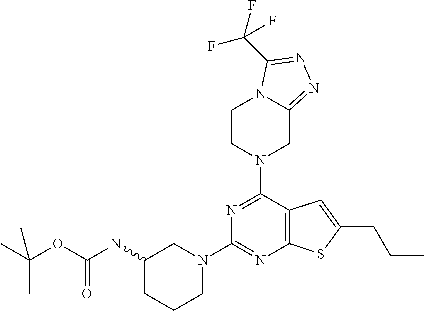

40 mg (0.1 mmol) of the compound obtained from Preparation Example 1-1-3 and 35 mg (0.2 mmol) of 3-aminopiperidine; 2 hydrochloride were dissolved in 2 mL of butanol, heated to 150° C. in a microwave reactor and stirred for 1 hour. The reaction solution was cooled to room temperature, distilled in vacuo and dissolved in 5 mL of methanol. To the solution was added 109 mg (0.5 mmol) of di-tert-butyl dicarbonate, and stirred for 16 hours. A solvent was removed by distillation in vacuo. The residue was purified by column chromatography using a mixed solution of methanol and dichloromethane in the ratio of 3:97 to obtain the title compound 46 mg (81%).

¹H NMR (400 MHz, CDCl₃); δ 6.79 (1H, s), 5.19 (2H, m), 4.48˜4.25 (4H, m), 4.20˜4.10 (2H, m), 3.63 (1H, m), 3.45 (1H, m), 3.30 (1H, m), 2.77 (2H, t), 1.91 (1H, m), 1.82˜1.53 (5H, m), 1.45 (9H, s), 1.00 (3H, t)

### Example 1-70

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine

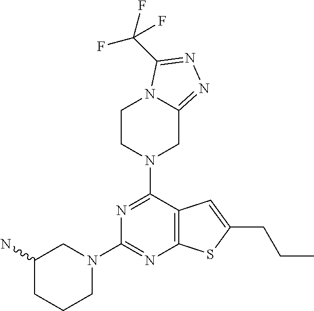

43 mg (98%) of the title compound was obtained according to the same method as Example 1-4 except that 46 mg (0.081 mmol) of the compound obtained from Preparation Example 1-70-1 was used instead of the compound obtained from Preparation Example 1-4-1.

¹H NMR (400 MHz, DMSO, d₆); δ 8.26 (3H, br s), 7.28 (1H, s), 5.17 (2H, m), 4.56 (1H, d), 4.37 (2H, t), 4.30-4.15 (5H, m), 3.69 (1H, m), 3.48 (1H, m), 3.37-3.10 (3H, m), 2.79 (2H, t), 2.01 (1H, m), 1.76 (1H, m), 1.73-1.58 (3H, m), 1.51 (3H, t)

### Example 1-71

### 7-[2-(2,3-Dihydro-indol-1-yl)-6-propyl-thieno[2,3-d]pyrimidin-4-yl]-3-trifluoromethyl-5,6,7,8-tetrahydro-[1,2,4]triazolo[4,3-a]pyrazine

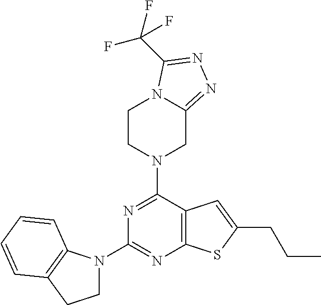

44 mg (90%) of the title compound was obtained according to the same method as Example 1-1 except that 24 mg (0.2 mmol) of indoline was used instead of piperazine-2-one.

¹H NMR (400 MHz, CDCl₃); δ 8.30 (1H, d), 7.26˜7.05 (2H, m), 6.92 (1H, t), 6.84 (1H, s), 5.23 (2H, s), 4.39 (2H, t), 4.30˜4.20 (4H, m), 3.18 (2H, t), 2.82 (2H, t), 1.75 (2H, m), 1.02 (3H, t)

### Example 1-72

### 7-[2-(1,3-Dihydro-isoindol-2-yl)-6-propyl-thieno[2,3-d]pyrimidin-4-yl]-3-trifluoromethyl-5,6,7,8-tetrahydro-[1,2,4]triazolo[4,3-a]pyrazine

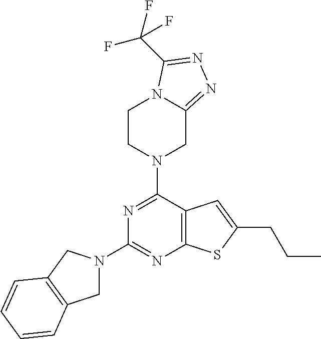

45 mg (92%) of the title compound was obtained according to the same method as Example 1-1 except that 24 mg (0.2 mmol) of isoindoline was used instead of piperazine-2-one.

¹H NMR (400 MHz, CDCl₃); δ 7.38˜7.26 (4H, m), 6.80 (1H, s), 5.24 (2H, s), 4.91 (4H, s), 4.39 (2H, t), 4.26 (2H, t), 2.79 (2H, t), 1.74 (2H, m), 1.01 (3H, t)

### Example 1-73

### 7-(2-Indol-1-yl-6-propyl-thieno[2,3-d]pyrimidin-4-yl)-3-trifluoromethyl-5,6,7,8-tetrahydro-[1,2,4]triazolo[4,3-a]pyrazine

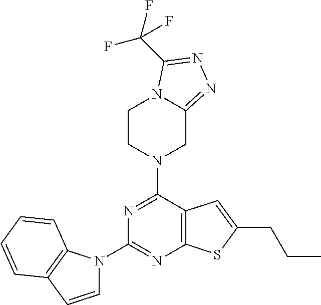

40 mg (77%) of the title compound was obtained according to the same method as Example 1-45 except that 23 mg (0.2 mmol) of indole was used instead of piperidine-3-carboxylic acid ethyl ester.

¹H NMR (400 MHz, CDCl₃); δ 8.72 (1H, d), 8.17 (1H, d), 7.61 (1H, d), 7.34 (1H, t), 7.22 (1H, t), 6.94 (1H, s), 6.65 (1H, d), 5.31 (2H, s), 4.41 (2H, t), 4.33 (2H, t), 2.88 (2H, t), 1.79 (2H, m), 1.05 (3H, t)

### Example 1-74

### 7,7′-(6-propylthieno[2,3-d]pyrimidine-2,4-diyl)bis[3-(trifluoromethyl)-5,6,7,8-tetrahydro[1,2,4]triazolo[4,3-a]pyrazine]

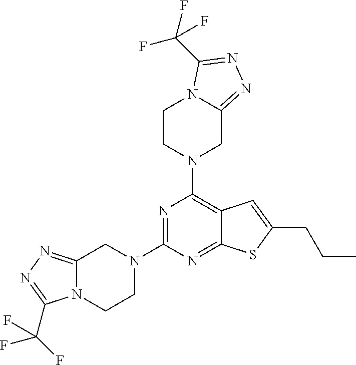

20 mg (36%) of the title compound was obtained according to the same method as Example 1-26 except that 46 mg (0.2 mmol) of the compound obtained from Preparation Example 1-1-2 was used instead of piperidine-4-ol; hydrochloride.

¹H NMR (400 MHz, CDCl₃); δ 6.87 (1H, s), 5.25 (2H, s), 4.40˜4.31 (4H, m), 4.28 (2H, t), 4.23 (2H, t), 2.81 (2H, t), 1.74 (2H, m), 1.01 (3H, t)

### Preparation Example 1-75-1

### 5,6,7,8-Tetrahydro-[1,2,4]triazolo[4,3-a]pyrazine

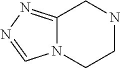

The title compound was synthesized according to the method in the known document (see Journal of Medicinal Chemistry 2005, 48(1), 141-151).

### Example 1-75

### 7-[2-(5,6-Dihydro-8H-[1,2,4]triazolo[4,3-a]pyrazin-7-yl)-6-propyl-thieno[2,3-d]pyrimidin-4-yl]-3-trifluoromethyl-5,6,7,8-tetrahydro-[1,2,4]triazolo[4,3-a]pyrazine

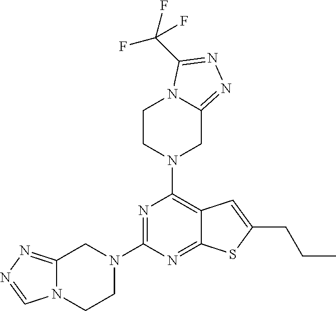

31 mg (63%) of the title compound was obtained according to the same method as Example 1-1 except that 25 mg (0.2 mmol) of the compound obtained from Preparation Example 1-75-1 was used instead of piperazine-2-one.

¹H NMR (400 MHz, CDCl₃); δ 8.15 (1H, s), 6.86 (1H, s), 5.24 (2H, s), 5.18 (2H, s), 4.37 (2H, t), 4.33˜4.24 (4H, m), 4.16 (2H, t), 2.81 (2H, t), 1.74 (2H, m), 1.01 (3H, t)

### Preparation Example 1-76-1

### 4,5,6,7-Tetrahydro-1H-pyrazolo[4,3-c]pyridine; dihydrochloride

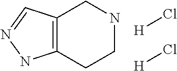

The title compound was synthesized according to the method in the known patent (see WO 2005/065779).

### Example 1-76

### 7-[6-Propyl-2-(1,4,6,7-tetrahydro-pyrazolo[4,3-c]pyridin-5-yl)-thieno[2,3-d]pyrimidin-4-yl]-3-trifluoromethyl-5,6,7,8-tetrahydro-[1,2,4]triazolo[4,3-a]pyrazine

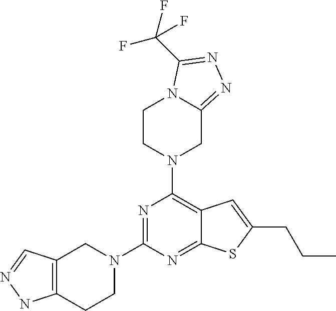

25 mg (51%) of the title compound was obtained according to the same method as Example 1-26 except that 39 mg (0.2 mmol) of the compound obtained from Preparation Example 1-76-1 was used instead of piperidine-4-ol; hydrochloride.

¹H NMR (400 MHz, CDCl₃); δ 7.43 (1H, s), 6.77 (1H, s), 5.18 (2H, s), 5.07 (2H, s), 4.35 (2H, t), 4.21 (2H, t), 4.14 (2H, t), 2.86 (2H, t), 2.78 (2H, t), 1.72 (2H, m), 0.99 (3H, t)

### Preparation Example 1-77-1

### 5,6,7,8-Tetrahydro-imidazo[1,2-a]pyrazine

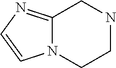

The title compound was synthesized according to the method in the known patent (see WO 03/004498).

### Example 1-77

### 7-[2-(5,6-Dihydro-8H-imidazo[1,2-a]pyrazin-7-yl)-6-propyl-thieno[2,3-d]pyrimidin-4-yl]-3-trifluoromethyl-5,6,7,8-tetrahydro-[1,2,4]triazolo[4,3-a]pyrazine

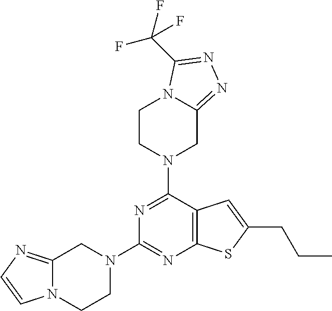

15 mg (31%) of the title compound was obtained according to the same method as Example 1-1 except that 24 mg (0.2 mmol) of the compound obtained from Preparation Example 1-77-1 was used instead of piperazine-2-one.

¹H NMR (400 MHz, CDCl₃); δ 7.05 (1H, s), 6.88 (1H, s), 6.84 (1H, s), 5.24 (2H, s), 5.03 (2H, s), 4.37 (2H, t), 4.32˜4.23 (4H, m), 4.08 (2H, t), 2.82 (2H, t), 1.73 (2H, m), 1.00 (3H, t)

### Preparation Example 1-78-1

### 2-Trifluoromethyl-5,6,7,8-tetrahydro-imidazo[1,2-a]pyrazine

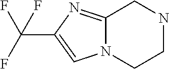

The title compound was synthesized according to the method in the known patent (see WO 03/004498).

### Example 1-78

### 7-[6-Propyl-2-(2-trifluoromethyl-5,6-dihydro-8H-imidazo[1,2-a]pyrazin-7-)-thieno[2,3-d]pyrimidin-4-yl]-3-trifluoromethyl-5,6,7,8-tetrahydro-[1,2,4]triazolo[4,3-a]pyrazine

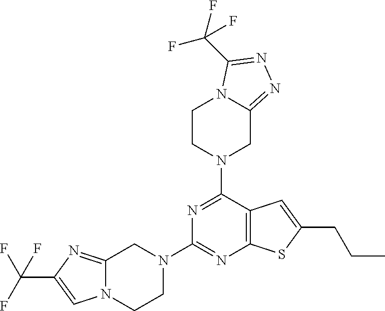

44 mg (79%) of the title compound was obtained according to the same method as Example 1-1 except that 38 mg (0.2 mmol) of the compound obtained from Preparation Example 1-78-1 was used instead of piperazine-2-one.

¹H NMR (400 MHz, CDCl₃); δ 7.23 (1H, s), 6.85 (1H, s), 5.24 (2H, s), 5.05 (2H, s), 4.36 (2H, t), 4.31 (2H, t), 4.26 (2H, t), 4.12 (2H, t), 2.80 (2H, t), 1.74 (2H, m), 1.00 (3H, t)

### Preparation Example 1-79-1

### 4,5,6,7-Tetrahydro-thieno[3,2-c]pyridine

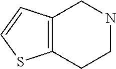

The title compound was synthesized according to the method in the known patent (see WO 2004/064778).

### Example 1-79

### 7-[2-(6,7-Dihydro-4H-thieno[3,2-c]pyridin-5-yl)-6-propyl-thieno[2,3-d]pyrimidin-4-yl]-3-trifluoromethyl-5,6,7,8-tetrahydro-[1,2,4]triazolo[4,3-a]pyrazine

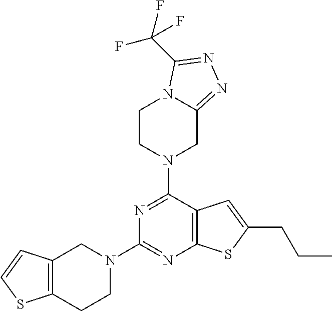

3.7 mg (9%) of the title compound was obtained according to the same method as Example 1-1 except that 25 mg (0.18 mmol) of the compound obtained from Preparation Example 1-79-1 was used instead of piperazine-2-one.

¹H NMR (500 MHz, CDCl₃); δ 7.12 (1H, d), 6.88 (1H, d), 6.76 (1H, s), 5.18 (2H, s), 4.84 (2H, s), 4.34 (2H, t), 4.20 (2H, t), 4.16 (2H, t), 2.93 (2H, m), 2.77 (2H, t), 1.73 (2H, m), 0.99 (3H, t)

### Preparation Example 1-80-1

### 2-Methyl-4-trifluoromethyl-5,6,7,8-tetrahydro-pyrido[3,4-d]pyrimidine; hydrochloride

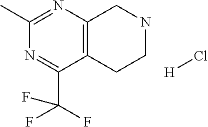

The title compound was synthesized according to the method in the known patent (see WO 2006/104356).

### Example 1-80

### 2-Methyl-7-[6-propyl-4-(3-trifluoromethyl-5,6-dihydro-8H-[1,2,4]triazolo[4,3-a]pyrazin-7-yl)-thieno[2,3-d]pyrimidin-2-yl]-4-trifluoromethyl-5,6,7,8-tetrahydro-pyrido[3,4-d]pyrimidine

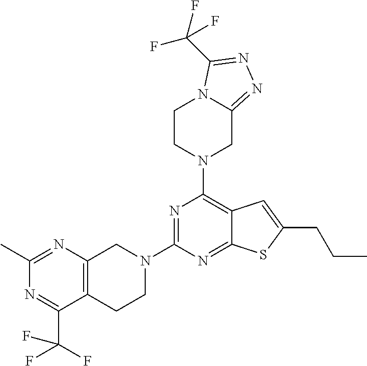

41 mg (71%) of the title compound was obtained according to the same method as Example 1-26 except that 51 mg (0.2 mmol) of the compound obtained from Preparation Example 1-80-1 was used instead of piperidine-4-ol; hydrochloride.

¹H NMR (400 MHz, CDCl₃); δ 6.82 (1H, s), 5.22 (2H, s), 5.04 (2H, s), 4.37 (2H, t), 4.25 (2H, t), 4.14 (2H, t), 3.06 (2H, t), 2.79 (2H, t), 2.78 (3H, s), 1.74 (2H, m), 1.00 (3H, t)

### Preparation Example 1-81-1

### 2-tert-Butoxycarbonylamino-3-(4-hydroxy-phenyl)-propionic acid methyl ester

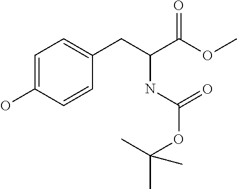

1.0 g (4.32 mmol) of 2-amino-3-(4-hydroxy-phenyl)-propionic acid methyl ester, 1.13 g (5.18 mmol) of di-tert-butyl dicarbonate and 1.5 mL (8.63 mmol) of diisopropylethylamine were dissolved in 30 mL of dichloromethane and stirred for 3 hours. The reaction mixture was distilled in vacuo and purified by column chromatography using a mixed solution of ethyl acetate and hexane in the ratio of 1:1 to obtain the title compound 1.1 g (86%).

¹H NMR (400 MHz, CDCl₃); δ 6.97 (2H, d), 6.73 (2H, d), 4.97 (1H, m), 4.53 (1H, m), 3.71 (3H, s), 3.00 (2H, m), 1.42 (9H, s)

### Preparation Example 1-81-2

### 3-(4-Acetoxy-phenyl)-2-tert-butoxycarbonylamino-propionic acid methyl ester

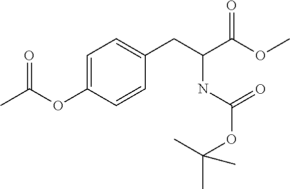

330 mg (1.12 mmol) of the compound obtained from Preparation Example 1-81-1 and 0.21 mL (2.23 mmol) of acetic anhydride were dissolved in 2 mL of pyridine and stirred for 16 hours. The reaction mixture was distilled in vacuo and purified by column chromatography using a mixed solution of ethyl acetate and hexane in the ratio of 1:1 to obtain the title compound 360 mg (96%).

¹H NMR (400 MHz, CDCl₃); δ 7.13 (2H, d), 7.01 (2H, d), 4.97 (1H, m), 4.57 (1H, m), 3.71 (3H, s), 3.09 (2H, m), 2.29 (3H, s), 1.42 (9H, s)

### Preparation Example 1-81-3

### 3-(4-Acetoxy-phenyl)-2-amino-propionic acid methyl ester; hydrochloride

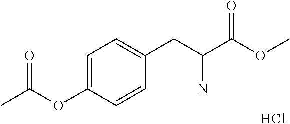

360 mg (1.07 mmol) of the compound obtained from Preparation Example 1-81-2 was dissolved in 2 mL of dichloromethane. To the solution was added 3 mL of 4.0 M HCl/dioxane solution and stirred at room temperature for 1 hour. To the reaction mixture was added 10 mL of diethyl ether. The obtained solid was filtered and dried to give the title compound 260 mg (89%).

¹H NMR (400 MHz, DMSO, d₆); δ 8.56 (3H, br, s), 7.26 (2H, d), 7.08 (2H, d), 4.30 (1H, t), 3.67 (3H, s), 3.13 (2H, m), 2.26 (3H, s)

### Preparation Example 1-81-4

### (3-(4-Acetoxy-phenyl)-2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid methyl ester

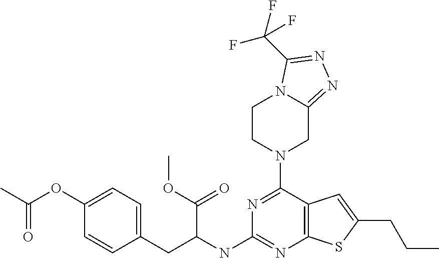

40 mg (53%) of the title compound was obtained according to a method similar to Example 1-45, using 50 mg (0.124 mmol) of the compound obtained from Preparation Example 1-1-3 and 67.95 mg (0.248 mmol) of the compound obtained from Preparation Example 1-81-3.

Mass: M+H 604

### Example 1-81

### 3-(4-Hydroxy-phenyl)-2-[6-propyl-4-(3-trifluoromethyl-5,6-dihydro-8H-[1,2,4]triazolo[4,3-a]pyrazin-7-yl)-thieno[2,3-d]pyrimidin-2-ylamino]-propionic acid

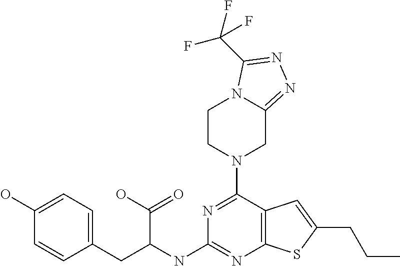

40 mg (0.066 mmol) of the compound obtained from Preparation Example 1-81-4 was dissolved in a mixed solution of tetrahydrofuran, water and methanol in the ratio of 5:3:1. To the solution was added 11.12 mg (0.265 mmol) of lithium hydroxide, and stirred for 6 hours at room temperature. The reaction mixture was distilled in vacuo and purified by column chromatography using ethyl acetate to obtain the title compound 10 mg (28%).

¹H NMR (400 MHz, MeOD); δ 7.08 (2H, d), 7.05 (1H, s), 6.67 (2H, d), 5.17 (2H, s), 4.67 (1H, s), 4.40 (2H, m), 4.23 (2H, m), 3.16 (1H, m), 2.98 (1H, m), 2.81 (2H, t), 1.73 (2H, m), 1.01 (3H, t)

### Example 1-82

### 2,2,2-Trifluoro-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-ethanone

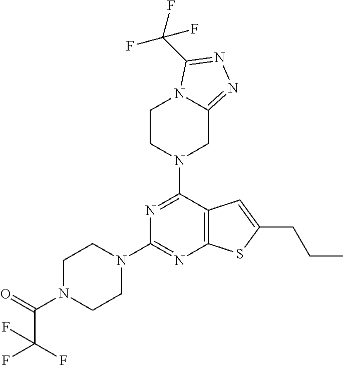

10 mg (24%) of the title compound was obtained according to a method similar to Example 1-1, using 30 mg (0.074 mmol) of the compound obtained from Preparation Example 1-1-3 and 32.56 mg (0.149 mmol) of 2,2,2-trifluoro-1-piperazine-1-yl-ethanone; hydrochloride.

¹H NMR (400 MHz, CDCl₃); δ 6.82 (1H, s), 5.20 (2H, s), 4.35 (2H, m), 4.23 (2H, m), 3.88 (4H, s), 2.79 (2H, t), 1.72 (2H, m), 1.00 (3H, t)

### Preparation Example 1-83-1

### 1-Benzhydryl-azetidin-3-ol

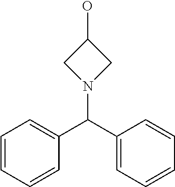

The title compound was synthesized according to the method in the known document (see J. Org. Chem., 1991, 56(24), 6729-6730).

### Preparation Example 1-83-2

### 3-Hydroxy-azetidine-1-carboxylic acid tert-butyl ester

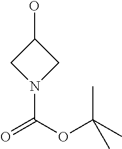

1.8 g (7.52 mmol) of the compound obtained from Preparation Example 1-83-1 was dissolved in 35 mL of methanol. To the solution was added 1.81 g (8.27 mmol) of di-tert-butyl dicarbonate, 0.8 g (7.9 mmol) of triethylamine, and palladium which is absorbed to active carbon (Pd/C) (10%, 0.18 g), and stirred under hydrogen condition for 16 hours. The reaction mixture was filtered through Celite, distilled in vacuo to remove a solvent and purified by column chromatography using a mixed solution of hexane and ethyl acetate in the ratio of 1:1 to obtain the title compound 1.06 g (80%).

¹H NMR (400 MHz, CDCl₃); δ 4.58 (1H, m), 4.15 (2H, dd), 3.80 (2H, dd), 2.08 (1H, d), 1.44 (9H, s)

### Preparation Example 1-83-3

### 3-Azido-azetidine-1-carboxylic acid tert-butyl ester

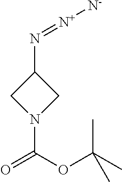

1.06 g (6.12 mmol) of the compound obtained from Preparation Example 1-83-2 and 2.0 g (7.65 mmol) of triphenylphosphine were dissolved in 40 mL of tetrahydrofuran. To the solution was added 1.61 g (7.96 mmol) of diisopropyl azodicarboxylate and 2.1 g (7.96 mmol) of diphenylphosphoryl azide, and stirred for 16 hours. The reaction mixture was distilled in vacuo to remove a solvent and purified by column chromatography using a mixed solution of hexane and ethyl acetate in the ratio of 9:1 to obtain the title compound 1.21 g (99%).

¹H NMR (400 MHz, CDCl₃); δ 4.25˜4.14 (3H, m), 3.89 (2H, m), 1.44 (9H, s)

### Preparation Example 1-83-4

### 3-Amino-azetidine-1-carboxylic acid tert-butyl ester

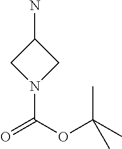

1.29 g (6.51 mmol) of the compound obtained from Preparation Example 1-83-3 was dissolved in 20 mL of methanol. To the solution was added palladium which is absorbed to active carbon (Pd/C) (10%, 0.13 g), and stirred under hydrogen condition for 3 hours. The reaction mixture was filtered through Celite, distilled in vacuo to remove a solvent and purified by column chromatography using a mixed solution of methanol and dichloromethane in the ratio of 10:90 to obtain the title compound 0.82 g (73%).

¹H NMR (400 MHz, CDCl₃); δ 4.14 (2H, dd), 3.76 (1H, m), 3.58 (2H, dd), 1.44 (9H, s)

### Preparation Example 1-83-5

### 3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-azetidine-1-carboxylic acid tert-butyl ester

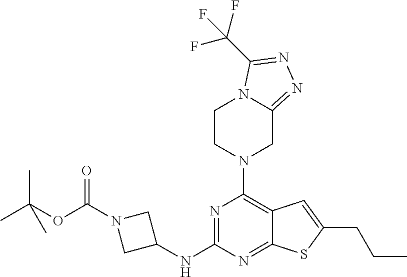

16 mg (30%) of the title compound was obtained according to the same method as Example 1-45 except that 34 mg (0.2 mmol) of the compound obtained from Preparation Example 1-83-4 was used instead of piperidine-3-carboxylic acid ethyl ester.

¹H NMR (400 MHz, CDCl₃); δ 6.96 (1H, s), 5.34 (1H, m), 5.30 (2H, s), 4.42-4.28 (6H, m), 4.05 (2H, dd), 2.84 (2H, t), 1.75 (2H, m), 1.45 (9H, s), 1.01 (3H, t)

### Example 1-83

### Azetidin-3-yl-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

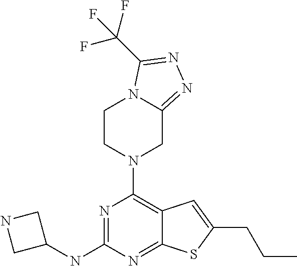

14 mg (93%) of the title compound was obtained according to the same method as Example 1-4 except that 16 mg (0.075 mmol) of the compound obtained from Preparation Example 1-83-5 was used instead of the compound obtained from Preparation Example 1-4-1.

¹H NMR (400 MHz, DMSO, d₆); δ 9.27 (1H, br s), 9.08 (1H, br s), 7.45 (1H, s), 5.39 (1H, m), 5.22 (2H, s), 4.46-4.26 (6H, m), 4.05 (1H, m), 2.85 (2H, t), 1.68 (2H, m), 0.95 (3H, t)

### Preparation Example 1-84-1

### 3-Acetoxy-azetidine-1-carboxylic acid tert-butyl ester

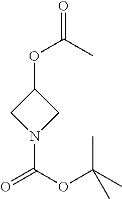

0.27 g (1.559 mmol) of the compound obtained from Preparation Example 1-83-2, 0.61 g (4.677 mmol) of diisopropylethylamine and a catalytic amount of 4-dimethylaminopyridine were dissolved in 7 mL of dichloromethane and cooled to 0° C. To the solution was added 0.32 g (2 mmol) of acetic anhydride and stirred at room temperature for 16 hours. The reaction mixture was distilled in vacuo to remove a solvent and purified by column chromatography using a mixed solution of hexane and ethyl acetate in the ratio of 3:1 to obtain the title compound 0.33 g (98%).

¹H NMR (400 MHz, CDCl₃); δ 5.12 (1H, m), 4.23 (2H, dd), 4.12 (2H, dd), 2.09 (3H, s), 1.44 (9H, s)

### Example 1-84

### Acetic Acid

### 1-[6-propyl-4-(3-trifluoromethyl-5,6-dihydro-8H-[1,2,4]triazolo[4,3-a]pyrazin-7-yl)-thieno[2,3-d]pyrimidin-2-yl]-azetidin-3-yl ester

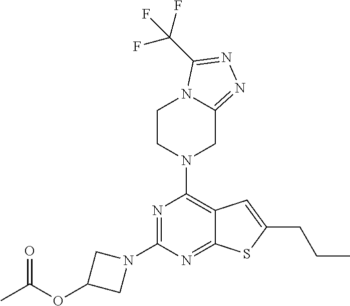

24 mg (50%) of the title compound was obtained according to the same method as Example 1-56 except that 43 mg (0.2 mmol) of the compound obtained from Preparation Example 1-84-1 was used instead of the compound obtained from Preparation Example 1-56-3.

¹H NMR (400 MHz, CDCl₃); δ 6.81 (1H, s), 5.29 (1H, m), 5.20 (2H, m), 4.46 (2H, dd), 4.35 (2H, t), 4.21 (2H, t), 4.07 (2H, dd), 2.78 (2H, t), 2.11 (3H, s), 1.72 (2H, m), 1.00 (3H, t)

### Example 1-85

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-azetidin-3-ol

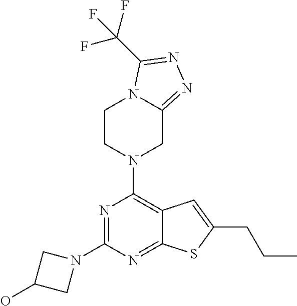

21 mg (0.044 mmol) of the compound obtained from Example 1-84 was dissolved in 3 mL of tetrahydrofuran and 0.5 mL of methane. To the solution was added 0.13 mL (0.13 mmol) of 1.0 M sodium hydroxide, and stirred for 1 hour. The reaction mixture was distilled in vacuo to remove a solvent and purified by column chromatography using a mixed solution of hexane and ethyl acetate in the ratio of 1:3 to obtain the title compound 15 mg (79%).

¹H NMR (400 MHz, CDCl₃); δ 6.79 (1H, s), 5.13 (2H, s), 4.77 (1H, m), 4.41˜4.30 (4H, m), 4.20 (2H, t), 3.98 (2H, dd), 2.77 (2H, t), 1.72 (2H, m), 0.99 (3H, t)

### Example 1-86

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid amide

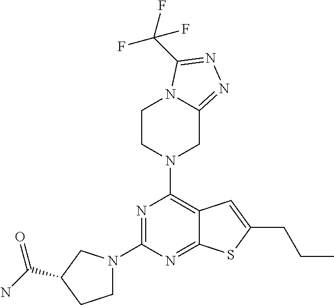

48 mg (0.10 mmol) of the compound obtained from Example 1-57, 6 mg (0.12 mmol) of ammonium chloride, 23 mg (0.12 mmol) of EDC and 20 mg (0.15 mmol) of HOBT were dissolved in 5 mL of N,N-dimethylformamide and cooled to 0° C. To the solution was added dropwise 65 mg (0.50 mmol) of diisopropylethylamine. After reaction at room temperature for 16 hours, the reaction mixture was distilled in vacuo, diluted with ethyl acetate and washed with water and brine. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 36 mg (75%).

¹H NMR (400 MHz, CDCl₃); δ 6.77 (1H, s), 5.71 (1H, br s), 5.65 (1H, br s), 5.17 (2H, s), 4.33 (2H, t), 4.20 (2H, t), 3.93˜3.72 (3H, m), 3.59 (1H, m), 3.01 (1H, m), 2.77 (2H, t), 2.27 (2H, m), 1.72 (2H, m), 0.99 (3H, t)

### Example 1-87

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid methylamide

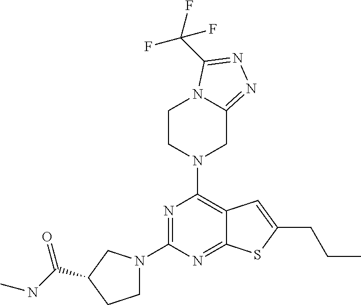

36 mg (73%) of the title compound was obtained according to the same method as Example 1-86 except that 8 mg (0.12 mmol) of methylamine; hydrochloride was used instead of ammonium chloride.

¹H NMR (400 MHz, CDCl₃); δ 6.76 (1H, s), 5.85 (1H, br s), 5.15 (2H, m), 4.33 (2H, t), 4.19 (2H, t), 3.90-3.67 (3H, m), 3.55 (1H, m), 2.96 (1H, m), 2.85 (3H, d), 2.76 (2H, t), 2.24 (2H, m), 1.71 (2H, m), 0.99 (3H, t)

### Example 1-88

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid dimethylamide

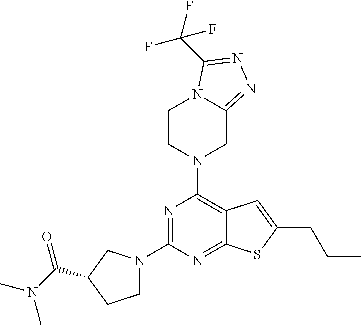

39 mg (76%) of the title compound was obtained according to the same method as Example 1-86 except that 10 mg (0.12 mmol) of methylamine; hydrochloride was used instead of ammonium chloride.

¹H NMR (400 MHz, CDCl₃); δ 6.77 (1H, s), 5.17 (2H, s), 4.33 (2H, t), 4.19 (2H, t), 3.90 (1H, m), 3.82 (1H, m), 3.72 (1H, m), 3.57 (1H, m), 3.36 (1H, m), 3.13 (3H, s), 2.99 (3H, s), 2.77 (2H, t), 2.32 (1H, m), 2.17 (1H, m), 1.72 (2H, m), 0.99 (3H, t)

### Example 1-89

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid hydroxyamide

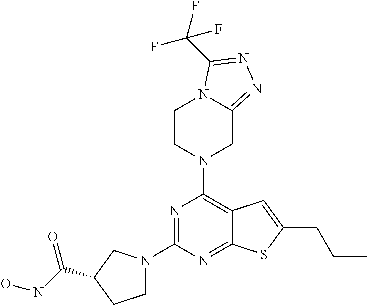

48 mg (0.10 mmol) of the compound obtained from Example 1-57, 10 mg (0.15 mmol) of hydroxylamine; hydrochloride and 57 mg (0.15 mmol) of HATU were dissolved in 5.0 mL of N,N-dimethylformamide. To the solution was added dropwise 65 mg (0.50 mmol) of diisopropylethylamine and stirred for 16 hours. The reaction mixture was distilled in vacuo, diluted with ethyl acetate and washed with water. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 23 mg (46%).

¹H NMR (400 MHz, CD₃OD); δ 7.05 (1H, s), 5.17 (2H, s), 4.41 (2H, s), 4.27 (2H, s), 3.82 (2H, m), 3.67 (1H, m), 3.56 (1H, m), 3.02 (1H, m), 2.82 (2H, m), 2.22 (2H, m), 1.73 (2H, m), 1.03 (3H, t)

### Example 1-90

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid (2-hydroxy-ethyl)-amide

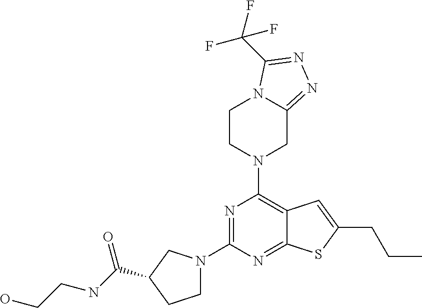

28 mg (54%) of the title compound was obtained according to the same method as Example 1-86 except that 7 mg (0.12 mmol) of ethanolamine was used instead of ammonium chloride.

¹H NMR (400 MHz, CDCl₃); δ 6.75 (1H, s), 6.60 (1H, t), 5.12 (2H, s), 4.31 (2H, t), 4.18 (2H, t), 3.88˜3.64 (5H, m), 3.57-3.36 (3H, m), 3.03 (1H, m), 2.75 (2H, t), 2.23 (2H, m), 1.71 (2H, m), 0.99 (3H, t)

### Example 1-91

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid (2-amino-ethyl)-amide

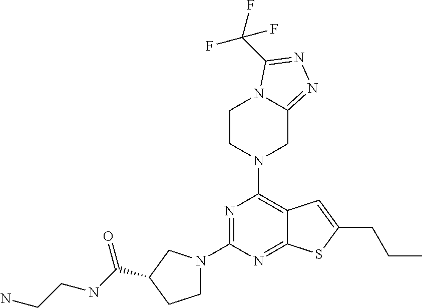

42 mg (0.087 mmol) of the compound obtained from Example 1-57, 14 mg (0.087 mmol) of (2-amino-ethyl)-carbamic acid tert-butyl ester, 20 mg (0.104 mmol) of EDC and 18 mg (0.131 mmol) of HOBT were dissolved in 5 mL of N,N-dimethylformamide and cooled to 0° C. To the solution was added dropwise 56 mg (0.435 mmol) of diiso-propylethylamine. After reaction at room temperature for 16 hours, the reaction mixture was distilled in vacuo, diluted with ethyl acetate and washed with water and brine. The organic layer was dried over anhydrous magnesium sulfate (MgSO₄) and distilled in vacuo. The residue was purified by column chromatography using a mixed solution of dichloromethane and methanol in the ratio of 90:10. To a solution of 42 mg of the purified compound in 1 mL of dichloromethane was added 3 mL of 4 M HCl/dioxane solution and stirred at room temperature for 30 minutes. The reaction mixture was dried in vacuo to obtain the title compound 40 mg (2-step 77%).

¹H NMR (400 MHz, DMSO, d₆); δ 8.43 (1H, s), 8.04 (3H, s), 7.33 (1H, s), 5.24 (2H, s), 4.40˜4.25 (4H, m), 3.88˜3.43 (4H, m), 3.36 (2H, m), 3.12 (1H, m), 2.89 (2H, m), 2.80 (2H, t), 2.20 (1H, m), 2.12 (1H, m), 1.67 (2H, m), 0.99 (3H, t)

### Example 1-92

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid amide

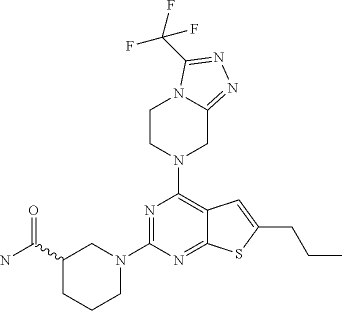

99 mg (0.20 mmol) of the compound obtained from Example 1-46, 13 mg (0.24 mmol) of ammonium chloride, 46 mg (0.24 mmol) of EDC and 41 mg (0.30 mmol) of HOBT were dissolved in 5 mL of N,N-dimethylformamide and cooled to 0° C. To the solution was added dropwise 65 mg (0.50 mmol) of diisopropylethylamine. After reaction at room temperature for 16 hours, the reaction mixture was distilled in vacuo, diluted with ethyl acetate and washed with water and brine. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 75 mg (76%).

¹H NMR (400 MHz, CDCl₃); δ 6.78 (1H, s), 6.41 (1H, br s), 5.75 (1H, br s), 5.15 (2H, m), 4.40˜4.10 (6H, m), 3.59 (1H, m), 3.40 (1H, m), 2.77 (2H, t), 2.40 (1H, m), 2.06˜1.87 (2H, m), 1.78-1.65 (2H, m), 1.53 (1H, m), 0.99 (3H, t)

### Example 1-93

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid methylamide

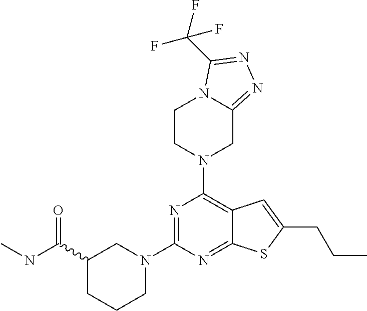

81 mg (79%) of the title compound was obtained according to the same method as Example 1-92 except that 16 mg (0.24 mmol) of methylamine; hydrochloride was used instead of ammonium chloride.

¹H NMR (400 MHz, CDCl₃); δ 6.77 (1H, s), 6.38 (1H, d), 5.14 (2H, m), 4.43-4.12 (6H, m), 3.45 (1H, m), 3.27 (1H, m), 2.81 (3H, d), 2.77 (2H, t), 2.31 (1H, m), 2.03˜1.85 (2H, m), 1.78˜1.64 (2H, m), 1.50 (1H, m), 0.99 (3H, t)

### Example 1-94

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid dimethylamide

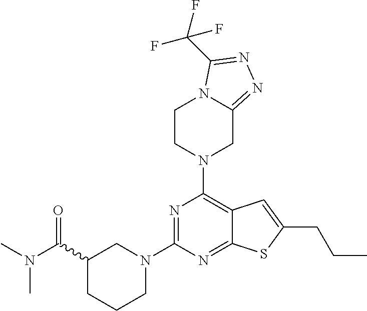

87 mg (83%) of the title compound was obtained according to the same method as Example 1-92 except that 20 mg (0.24 mmol) of dimethylamine; hydrochloride was used instead of ammonium chloride.

¹H NMR (400 MHz, CDCl₃); δ 6.77 (1H, s), 5.15 (2H, m), 4.79 (1H, d), 4.73 (1H, d), 4.35 (2H, t), 4.18 (2H, m), 3.12 (3H, s), 3.04 (1H, m), 2.98 (3H, s), 2.89 (1H, m), 2.77 (2H, t), 2.71 (1H, m), 2.00-1.66 (5H, m), 1.50 (1H, m), 1.00 (3H, t)

### Example 1-95

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidine-3-carboxylic acid hydroxyamide

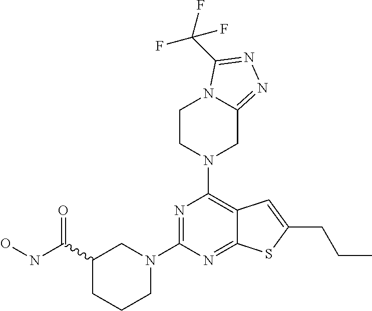

60 mg (0.12 mmol) of the compound obtained from Example 1-46, 10 mg (0.14 mmol) of hydroxylamine; hydrochloride and 68 mg (0.18 mmol) of HATU were dissolved in 5.0 mL of N,N-dimethylformamide. To the solution was added dropwise 78 mg (0.60 mmol) of diisopropylethylamine and stirred for 16 hours. The reaction mixture was distilled in vacuo, diluted with ethyl acetate and washed with water. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 23 mg (52%).

¹H NMR (400 MHz, CDCl₃); δ 10.5 (1H, br s), 6.75 (1H, s), 5.11 (2H, m), 4.27 (2H, s), 4.13 (2H, s), 3.96 (1H, m), 3.71 (1H, m), 3.51 (1H, m), 3.17 (1H, m), 2.75 (2H, t), 2.44 (1H, m), 2.07 (1H, m), 1.87 (1H, m), 1.68 (2H, m), 1.59 (1H, m), 1.48 (1H, m), 0.99 (3H, t)

### Example 1-96

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-aziridine-2-carboxylic acid methyl ester

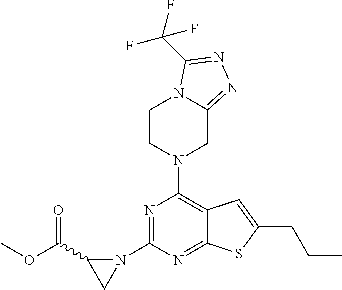

38 mg (41%) of the title compound was obtained according to the same method as Example 1-45 except that 30 mg (0.30 mmol) of aziridine-2-carboxylic acid methyl ester was used instead of piperidine-3-carboxylic acid ethyl ester.

¹H NMR (400 MHz, CDCl₃); δ 6.94 (1H, s), 5.27 (2H, s), 4.42˜4.27 (4H, m), 3.78 (3H, s), 3.22 (1H, dd), 2.84 (2H, t), 2.78 (1H, d), 2.67 (1H, d), 1.75 (2H, m), 1.01 (3H, t)

### Example 1-97

### Dimethyl-{1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amine

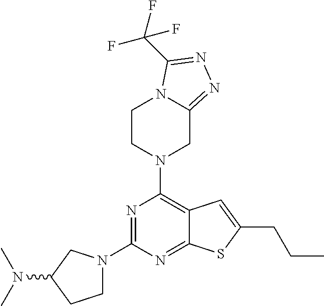

48 mg (100%) of the title compound was obtained according to the same method as Example 1-1 except that 23 mg (0.20 mmol) of 3-(dimethylamino)pyrrolidine was used instead of piperazine-2-one.

¹H NMR (400 MHz, CDCl₃); δ 6.76 (1H, s), 5.17 (2H, s), 4.35 (2H, t), 4.19 (2H, t), 3.92 (1H, m), 3.81 (1H, m), 3.50 (1H, m), 3.32 (1H, m), 2.79 (1H, m), 2.77 (2H, t), 2.33 (6H, s), 2.20 (1H, m), 1.90 (1H, m), 1.72 (2H, m), 0.99 (3H, t)

### Example 1-98

### [6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl-amine

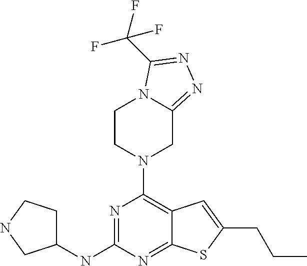

40 mg (0.10 mmol) of the compound obtained from Preparation Example 1-1-3, 37 mg (0.20 mmol) of 3-amino-1-N-Boc-pyrrolidine, 2.2 mg (0.01 mmol) of palladium-acetate (II), 7.5 mg (0.012 mmol) of BINAP and 49 mg (0.15 mmol) of cesium carbonate were dissolved in 5 mL of toluene, and stirred under reflux for 3 hours. The reaction solution was cooled to room temperature, filtered through Celite, distilled in vacuo to remove a solvent and purified by column chromatography using a mixed solution of hexane and ethyl acetate in the ratio of 1:2. To a solution of 29 mg of the purified compound in 1 mL of dichloromethane was added 3 mL of 4 M HCl/dioxane solution and stirred at room temperature for 30 minutes. The reaction mixture was dried in vacuo to obtain the title compound 26 mg (2-step 49%).

¹H NMR (400 MHz, DMSO, d₆); δ 9.34 (2H, br s), 7.68 (1H, br s), 7.31 (1H, s), 5.21 (2H, s), 4.56 (1H, m), 4.42˜4.24 (4H, m), 3.54˜3.07 (4H, m), 2.80 (2H, t), 2.20 (1H, m), 1.99 (1H, m), 1.67 (2H, m), 0.95 (3H, t)

### Preparation Example 1-99-1

### 2-(tert-Butyl-dimethyl-silanyloxy)-ethanol

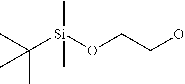

The title compound was synthesized according to the method in the known document (see J. Org. Chem., 51, 3388 (1986)).

### Preparation Example 1-99-2

### (tert-Butyl-dimethyl-silanyloxy)-acetaldehyde

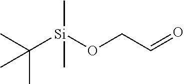

176 mg (1.0 mmol) of the compound obtained from Preparation Example 1-99-1 was dissolved in 10 mL of dichloromethane. To the solution was added 176 mg (1.5 mmol) of N-methylmorpholine N-oxide and 18 mg (0.05 mmol) of TPAP, and stirred for 30 minutes. The reaction mixture was distilled in vacuo to remove a solvent and purified by column chromatography using a mixed solution of hexane and ethyl acetate in the ratio of 9:1 to obtain the title compound 35 mg (20%).

¹H NMR (400 MHz, CDCl₃); δ 9.70 (1H, s), 4.21 (2H, s), 0.93 (9H, s), 0.11 (6H, s)

### Preparation Example 1-99-3

### [2-(tert-Butyl-dimethyl-silanyloxy)-ethyl]-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amine

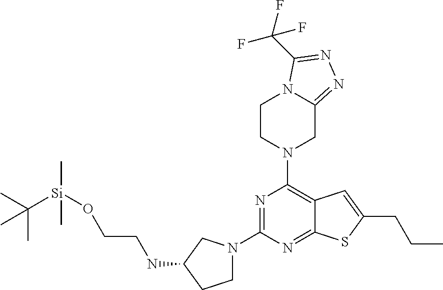

105 mg (0.20 mmol) of the compound obtained from Preparation Example 1-99-2 and 35 mg (0.20 mmol) of the compound obtained from Example 1-14 were dissolved in 5 mL of dichloroethane. To the solution was added dropwise 64 mg (0.30 mmol) of sodium triacetoxyborohydride and stirred for 16 hours. The reaction mixture was distilled in vacuo, diluted with dichloromethane and washed with water. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 32 mg (26%).

¹H NMR (400 MHz, CDCl₃); δ 6.69 (1H, s), 5.11 (2H, s), 4.28 (2H, t), 4.12 (2H, t), 3.77 (1H, m), 3.68 (3H, m), 3.52 (1H, m), 3.40 (1H, m), 3.31 (1H, m), 2.78˜2.64 (4H, m), 2.13 (1H, m), 1.78 (1H, m), 1.65 (2H, m), 0.93 (3H, t), 0.82 (9H, s), 0.01 (6H, s)

### Example 1-99

### 2-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamino}-ethanol

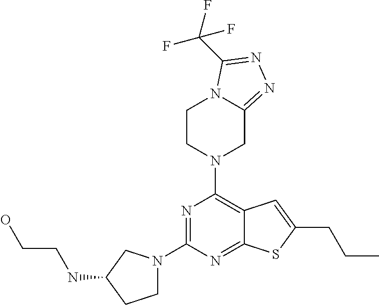

32 mg (0.052 mmol) of the compound obtained from Preparation Example 1-99-3 was dissolved in 3 mL of tetrahydrofuran. To the solution was added 0.16 mL (0.16 mmol) of 1 M tetrabutylammonium fluoride and stirred for 2 hours. The reaction mixture was diluted with dichloromethane and washed with water twice. The organic layer was dried over anhydrous magnesium sulfate (MgSO₄) and distilled in vacuo. The residue was purified by column chromatography using a mixed solution of dichloromethane and methanol in the ratio of 85:15 to obtain the title compound 7 mg (27%).

¹H NMR (400 MHz, CDCl₃); δ 6.76 (1H, s), 5.17 (2H, s), 4.33 (2H, t), 4.19 (2H, t), 3.88˜3.43 (7H, m), 2.90 (2H, t), 2.13 (1H, m), 2.77 (2H, t), 2.22 (1H, m), 1.94 (1H, m), 1.72 (2H, m), 0.99 (3H, t)

### Example 1-100

### 2-Hydroxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

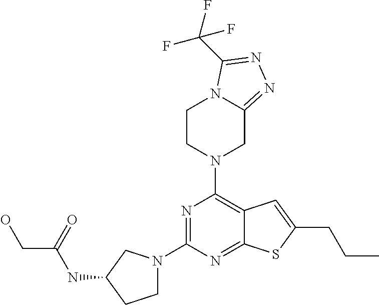

53 mg (0.10 mmol) of the compound obtained from Example 1-14, 9 mg (0.12 mmol) of glycolic acid, 23 mg (0.12 mmol) of EDC and 20 mg (0.15 mmol) of HOBT were dissolved in 5 mL of N,N-dimethylformamide and cooled to 0° C. To the solution was added dropwise 65 mg (0.50 mmol) of diisopropylethylamine. After reaction at room temperature for 16 hours, the reaction mixture was distilled in vacuo, diluted with ethyl acetate and washed with water and brine. The organic layer was dried over anhydrous magnesium sulfate (MgSO₄) and distilled in vacuo. The residue was purified by column chromatography using a mixed solution of dichloromethane and methanol in the ratio of 90:10 to obtain the title compound 39 mg (76%).

¹H NMR (400 MHz, CDCl₃); δ 6.86 (1H, d), 6.77 (1H, s), 5.15 (2H, m), 4.63 (1H, m), 4.31 (2H, t), 4.18 (2H, t), 4.12 (2H, s), 3.85 (1H, m), 3.74˜3.60 (2H, m), 3.50 (1H, m), 2.77 (2H, t), 2.30 (1H, m), 2.00 (1H, m), 1.72 (2H, m), 0.99 (3H, t)

### Example 1-101

### 2-Amino-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

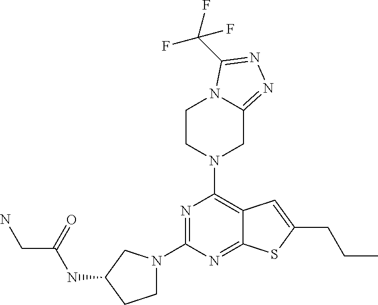

53 mg (0.10 mmol) of the compound obtained from Example 1-14, 21 mg (0.12 mmol) of Boc-glycine, 23 mg (0.12 mmol) of EDC and 20 mg (0.15 mmol) of HOBT were dissolved in 5 mL of N,N-dimethylformamide and cooled to 0° C. To the solution was added dropwise 65 mg (0.50 mmol) of diisopropylethylamine. After reaction at room temperature for 16 hours, the reaction mixture was distilled in vacuo, diluted with ethyl acetate and washed with water and brine. The organic layer was dried over anhydrous magnesium sulfate (MgSO₄), distilled in vacuo and purified by column chromatography using a mixed solution of dichloromethane and methanol in the ratio of 95:5. To a solution of 42 mg of the purified compound in 1 mL of dichloromethane was added 3 mL of 4 M HCl/dioxane solution and stirred at room temperature for 30 minutes. The reaction mixture was dried in vacuo to obtain the title compound 50 mg (2-step 86%).

¹H NMR (400 MHz, DMSO, d₆); δ 8.90 (1H, d), 8.14 (3H, br s), 6.77 (1H, s), 7.35 (1H, s), 5.24 (1H, s), 4.47˜4.27 (5H, m), 3.80-3.43 (4H, m), 2.81 (2H, t), 2.12 (1H, m), 1.97 (1H, m), 1.67 (2H, m), 0.95 (3H, t)

### Example 1-102

### 2-Methoxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

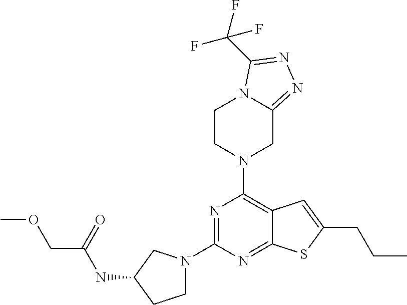

38 mg (73%) of the title compound was obtained according to the same method as Example 1-100 except that 11 mg (0.12 mmol) of methoxyacetic acid was used instead of glycolic acid.

¹H NMR (400 MHz, CDCl₃); δ 6.79 (1H, s), 6.65 (1H, d), 5.19 (2H, s), 4.64 (1H, m), 4.34 (2H, t), 4.21 (2H, t), 3.96˜3.83 (3H, m), 3.70 (2H, t), 3.50 (1H, m), 3.40 (3H, s), 2.78 (2H, t), 2.30 (1H, m), 1.99 (1H, m), 1.72 (2H, m), 1.00 (3H, t)

### Example 1-103

### 2-Cyano-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

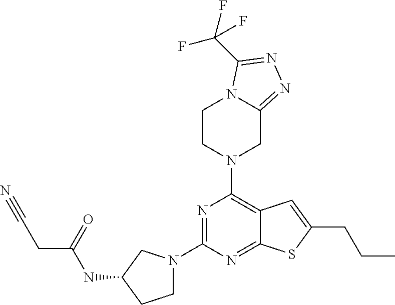

25 mg (48%) of the title compound was obtained according to the same method as Example 1-100 except that 10 mg (0.12 mmol) of cyanoacetic acid was used instead of glycolic acid.

¹H NMR (400 MHz, CDCl₃); δ 7.13 (1H, d), 6.72 (1H, s), 5.03 (2H, s), 4.59 (1H, m), 4.18 (2H, br s), 4.07 (2H, br s), 3.78 (1H, dd), 3.66 (2H, dd), 3.58 (1H, dd), 3.41 (2H, s), 2.78 (2H, t), 2.27 (1H, m), 2.08 (1H, m), 1.73 (2H, m), 1.01 (3H, t)

### Example 1-104

### 3,3,3-Trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

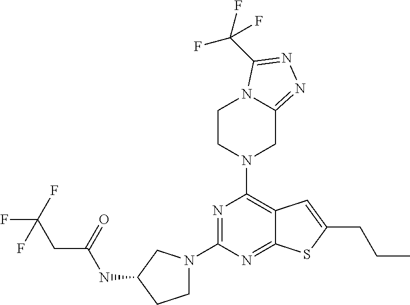

45 mg (80%) of the title compound was obtained according to the same method as Example 1-100 except that 15 mg (0.12 mmol) of 3,3,3-trifluoropropionic acid was used instead of glycolic acid.

¹H NMR (400 MHz, CDCl₃); δ 6.83 (1H, d), 6.72 (1H, s), 5.03 (2H, s), 4.62 (1H, m), 4.26˜4.01 (4H, m), 3.77 (1H, dd), 3.65 (2H, m), 3.54 (1H, dd), 3.10 (2H, q), 2.78 (2H, t), 2.26 (1H, m), 2.07 (1H, m), 1.73 (2H, m), 1.01 (3H, t)

### Example 1-105

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

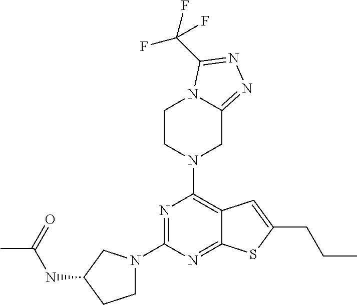

53 mg (0.10 mmol) of the compound obtained from Example 1-14 was dissolved in 5 mL of dichloromethane and cooled to 0° C. To the solution was added 20 mg (0.20 mmol) of triethylamine and 15 mg (0.15 mmol) of acetic anhydride. After adding a catalytic amount of 4-dimethylaminopyridine, the temperature was elevated to room temperature and stirred for 16 hours. The reaction mixture was diluted with dichloromethane and washed with water and brine. The reaction mixture distilled in vacuo and purified by column chromatography using a mixed solution of dichloromethane and methanol in the ratio of 95:5 to obtain the title compound 36 mg (73%).

¹H NMR (400 MHz, CDCl₃); δ 6.77 (1H, s), 5.96 (1H, d), 5.14 (2H, s), 4.58 (1H, m), 4.30 (2H, t), 4.17 (2H, m), 3.81 (1H, dd), 3.65 (2H, t), 3.48 (1H, dd), 2.77 (2H, t), 2.26 (1H, m), 2.00 (3H, s), 1.97 (1H, m), 1.72 (2H, m), 1.00 (3H, t)

### Example 1-106

### 2,2,2-Trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

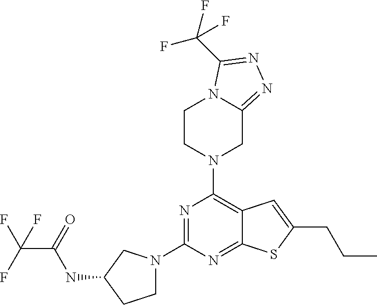

46 mg (84%) of the title compound was obtained according to the same method as Example 1-105 except that 32 mg (0.15 mmol) of trifluoroacetic anhydride was used instead of acetic anhydride.

¹H NMR (400 MHz, CDCl₃); δ 7.24 (1H, d), 6.74 (1H, s), 5.06 (2H, s), 4.64 (1H, m), 4.30˜4.00 (4H, m), 3.83 (1H, dd), 3.70 (2H, dd), 3.64 (1H, dd), 2.79 (2H, t), 2.33 (1H, m), 2.14 (1H, m), 1.73 (2H, m), 1.01 (3H, t)

### Example 1-107

### 2-Hydroxy-2-methyl-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

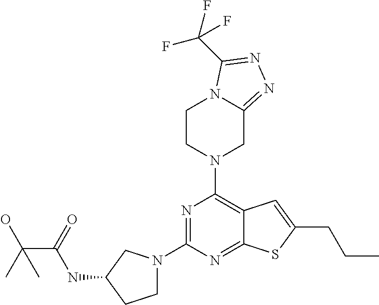

60 mg (0.115 mmol) of the compound obtained from Example 1-14, 18 mg (0.173 mmol) of 2-hydroxyisobutyric acid and 66 mg (0.173 mmol) of HATU were dissolved in 5.0 mL of N,N-dimethylformamide. To the solution was added 74 mg (0.575 mmol) of diisopropylethylamine and stirred for 16 hours. The reaction mixture was distilled in vacuo, diluted with ethyl acetate and washed with water. The organic layer was dried over anhydrous magnesium sulfate (MgSO₄) and distilled in vacuo. The residue was purified by column chromatography using a mixed solution of dichloromethane and methanol in the ratio of 92:8 to obtain the title compound 47 mg (76%).

¹H NMR (400 MHz, CDCl₃); δ 7.05 (1H, d), 6.80 (1H, s), 5.19 (2H, s), 4.59 (1H, m), 4.35 (2H, t), 4.23 (2H, m), 3.90 (1H, dd), 3.71 (2H, m), 3.48 (1H, dd), 2.80 (2H, t), 2.32 (1H, m), 2.00 (1H, m), 1.75 (2H, m), 1.50 (6H, m), 1.03 (3H, t)

### Example 1-108

### Cyclopropanecarboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

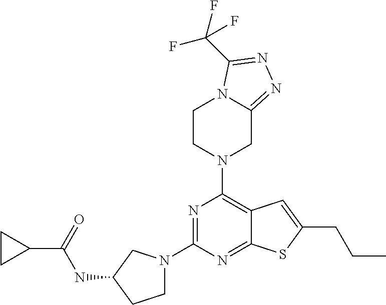

22 mg (37%) of the title compound was obtained according to the same method as Example 1-107 except that 15 mg (0.173 mmol) of cyclopropanecarboxylic acid was used instead of 2-hydroxyisobutyric acid.

¹H NMR (400 MHz, CDCl₃); δ 6.77 (1H, s), 6.16 (1H, d), 5.14 (2H, s), 4.61 (1H, m), 4.31 (2H, t), 4.16 (2H, m), 3.81 (1H, dd), 3.66 (2H, m), 3.50 (1H, dd), 2.77 (2H, t), 2.26 (1H, m), 1.99 (1H, m), 1.72 (2H, m), 1.37 (1H, m), 1.08˜0.92 (5H, m), 0.74 (3H, t)

### Example 1-109

### 3-Hydroxy-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

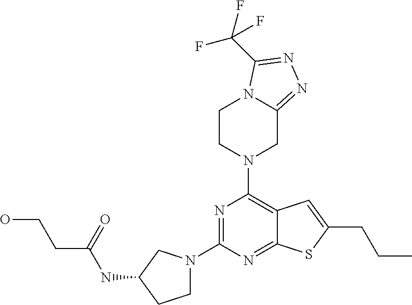

12 mg (23%) of the title compound was obtained according to the same method as Example 1-100 except that 11 mg (0.12 mmol) of 3-hydroxypropionic acid was used instead of glycolic acid.

¹H NMR (400 MHz, CDCl₃); δ 6.75 (1H, s), 6.54 (1H, d), 5.11 (2H, s), 4.58 (1H, m), 4.28 (2H, t), 4.15 (2H, m), 3.89 (2H, t), 3.82 (1H, dd), 3.65 (2H, m), 3.48 (1H, m), 3.30 (1H, br s), 2.77 (2H, t), 2.45 (2H, t), 2.26 (1H, m), 1.98 (1H, m), 1.72 (2H, m), 1.00 (3H, t)

### Example 1-110

### 3-Amino-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-propionamide

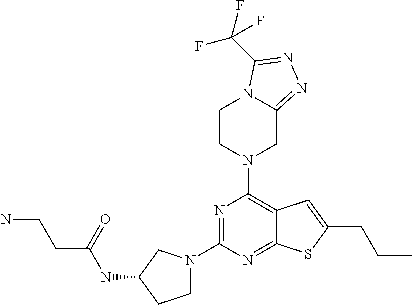

50 mg (2-step 23%) of the title compound was obtained according to the same method as Example 1-101 except that 23 mg (0.12 mmol) of 3-tert-butoxycarbonylaminopropionic acid was used instead of Boc-glycine.

¹H NMR (400 MHz, DMSO, d₆); δ 8.53 (1H, s), 7.94 (4H, br s), 7.34 (1H, d), 5.24 (2H, s), 4.43˜4.27 (5H, m), 3.83˜3.42 (4H, m), 2.98 (3H, m), 2.81 (2H, t), 2.61 (1H, t), 2.17 (1H, m), 1.95 (1H, m), 1.67 (2H, m), 0.95 (3H, t)

### Example 1-111

### Pyridine-2-carboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

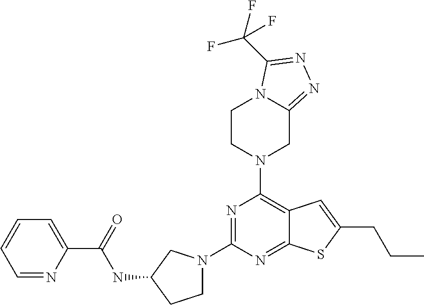

49 mg (88%) of the title compound was obtained according to the same method as Example 1-100 except that 15 mg (0.12 mmol) of picolinic acid was used instead of glycolic acid.

¹H NMR (400 MHz, CDCl₃); δ 8.51 (1H, d), 8.19 (2H, m), 7.85 (1H, t), 7.43 (1H, dd), 6.79 (1H, s), 5.20 (2H, s), 4.79 (1H, m), 4.35 (2H, t), 4.22 (2H, t), 3.97 (1H, dd), 3.75 (2H, m), 3.65 (1H, dd), 2.78 (2H, t), 2.39 (1H, m), 2.13 (1H, m), 1.72 (2H, m), 0.99 (3H, t)

### Example 1-112

### Furan-2-carboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

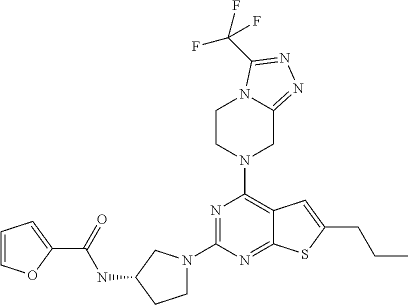

46 mg (84%) of the title compound was obtained according to the same method as Example 1-107 except that 17 mg (0.15 mmol) of 2-furoic acid was used instead of 2-hydroxyisobutyric acid.

¹H NMR (400 MHz, CDCl₃); δ 7.41 (1H, s), 7.12 (1H, d), 6.79 (1H, s), 6.55 (1H, d), 6.49 (1H, d), 5.18 (2H, s), 4.76 (1H, m), 4.34 (2H, t), 4.21 (2H, m), 3.93 (1H, dd), 3.73 (2H, m), 3.61 (1H, dd), 2.78 (2H, t), 2.36 (1H, m), 2.09 (1H, m), 1.72 (2H, m), 1.00 (3H, t)

### Example 1-113

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methanesulfonamide

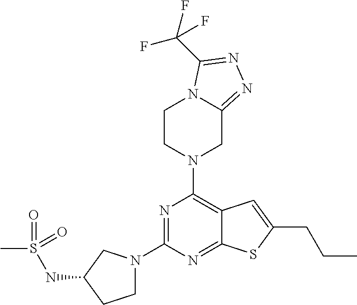

53 mg (0.10 mmol) of the compound obtained from Example 1-14 was dissolved in 3 mL of pyridine and cooled to 0° C. To the solution was added 14 mg (0.12 mmol) of methanesulfonyl chloride and the temperature was elevated to room temperature and stirred for 16 hours. The reaction mixture was diluted with ethyl acetate and washed with water and brine. The reaction mixture distilled in vacuo and purified by column chromatography using a mixed solution of dichloromethane and methanol in the ratio of 95:5 to obtain the title compound 31 mg (53%).

¹H NMR (400 MHz, CDCl₃); δ 6.78 (1H, s), 5.19 (1H, br s), 5.15 (2H, s), 4.29 (2H, t), 4.17 (2H, t), 4.13 (1H, m), 3.88 (1H, dd), 3.72 (1H, m), 3.67-53 (2H, m), 3.03 (3H, s), 2.77 (2H, t), 2.32 (1H, m), 2.05 (1H, m), 1.72 (2H, m), 1.00 (3H, t)

### Preparation Example 1-114-1

### 6-Oxa-3-aza-bicyclo[3.1.0]hexane-3-carboxylic acid tert-butyl ester

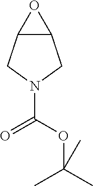

3.0 g (43.40 mmol) of 2,5-dihydro-1H-pyrrole and 14.21 g (65.10 mmol) of di tert-butyl dicarbonate were diluted with 10 mL of dichloromethane and stirred at room temperature for 2 hours. The reaction solution was distilled in vacuo and purified by column chromatography using a mixed solution of hexane and ethyl acetate in the ratio of 5:1. 7 g of the purified compound was dissolved in 20 mL of dichloromethane and cooled to 0° C. To the solution was added 14.98 g (86.80 mmol) of 3-chloro-benzenecarboperoxic acid and stirred at room temperature for 12 hours. The reaction mixture was distilled in vacuo, diluted with ethyl acetate and washed with water and brine. The organic layer was dried over anhydrous magnesium sulfate (MgSO₄), distilled in vacuo and purified by column chromatography using a mixed solution of hexane and ethyl acetate in the ratio of 5:1 to obtain the title compound 2.93 g (2-step 37%).

¹H NMR (400 MHz, CDCl₃); δ 3.80 (1H, d), 3.73 (1H, d), 3.66 (2H, d), 3.29 (2H, q), 1.44 (9H, s)

### Preparation Example 1-114-2

### 3-tert-Butoxycarbonylamino-4-hydroxy-pyrrolidine-1-carboxylic acid tert-butyl ester

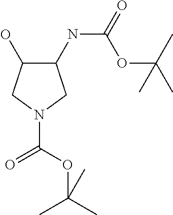

To 2.93 g (15.80 mmol) of the compound obtained from Preparation Example 1-114-1 was added 25 mL of ammonia water and stirred at 60° C. for 15 hours. The reaction solution was distilled in vacuo, diluted with dichloromethane and washed with water. The organic layer was dried over anhydrous magnesium sulfate (MgSO₄) and distilled in vacuo. To the residue was added 30 mL of dichloromethane and 14.21 g (65.10 mmol) of di tert-butyl dicarbonate and stirred for 4 hours. The reaction solution was distilled in vacuo and purified by column chromatography using a mixed solution of dichloromethane and methanol in the ratio of 95:5 to obtain the title compound 2.95 g (2-step 62%).

¹H NMR (400 MHz, CDCl₃); δ 4.67 (1H, br), 4.21 (1H, br), 3.91 (1H, br), 3.76 (2H, br), 3.27 (1H, br), 3.17 (1H, br), 1.44 (18H, s)

### Preparation Example 1-114-3

### 4-Amino-pyrrolidin-3-ol

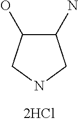

2.95 g (9.76 mmol) of the compound obtained from Preparation Example 1-114-2 was purified according to the same method as Example 1-4 to obtain the title compound 1.64 g (96%).

¹H NMR (400 MHz, DMSO, d₆); δ 8.75 (4H, br), 6.05 (1H, br), 4.38 (1H, br), 3.58 (1H, br), 3.54 (3H, d), 3.24 (1H, d), 3.08 (2H, d)

### Preparation Example 1-114-4

### {4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid tert-butyl ester

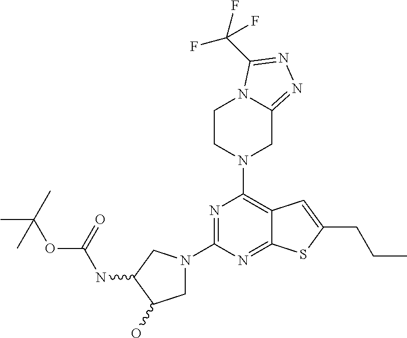

3.20 g (7.94 mmol) of the compound obtained from Preparation Example 1-1-3, 2.78 g (15.89 mmol) of the compound obtained from Preparation Example 1-114-3 and 5.14 g (39.68 mmol) of diisopropylethylamine were diluted with 40 mL of butanol, heated to 150° C. in a microwave reactor and stirred for 1 hour. The reaction solution was distilled in vacuo. To the reaction mixture was added 30 mL of dichloromethane and 2.60 g (11.90 mmol) of di tert-butyl dicarbonate and distilled in vacuo at 50° C. to complete the reaction. The reaction mixture was purified by column chromatography using a mixed solution of dichloromethane and ethyl acetate in the ratio of 4:1 to obtain the title compound 3.43 g (2-step 76%).

¹H NMR (400 MHz, CDCl₃); δ 6.78 (1H, s), 5.19 (2H, s), 4.81 (1H, br), 4.32 (2H, d), 4.19 (2H, d), 4.04 (2H, m), 3.93 (1H, m), 3.66 (1H, br), 3.53 (1H, m), 3.45 (1H, m), 2.76 (2H, t), 1.76 (2H, m), 1.53 (9H, s), 0.92 (3H, t)

### Example 1-114

### 4-Amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol

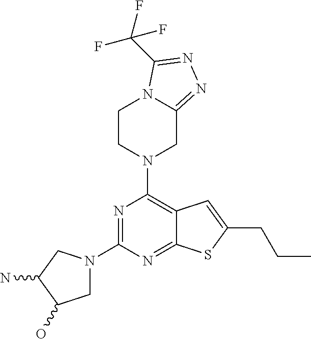

20 mg (0.035 mmol) of the compound obtained from Preparation Example 1-114-4 was purified according to the same method as Example 1-4 to obtain the title compound 17 mg (89%).

¹H NMR (400 MHz, DMSO, d₆); δ 7.27 (1H, s), 5.17 (2H, s), 4.52 (2H, br), 4.35 (2H, d), 4.25 (2H, br), 3.44 (1H, m), 3.34 (1H, m), 3.30 (1H, m), 3.11 (1H, m), 2.77 (2H, t), 2.17 (1H, m), 1.97 (1H, m), 1.63 (2H, m), 0.93 (3H, t)

### Preparation Example 1-115

### {4-Oxo-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid tert-butyl ester

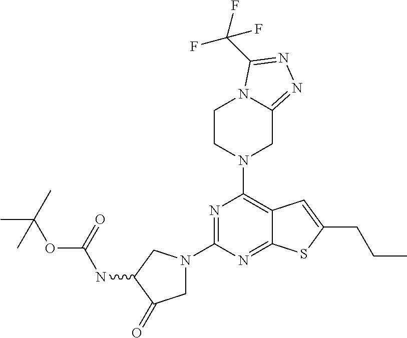

142 mg (0.035 mmol) of the compound obtained from Preparation Example 1-114-4 and 76 mg (0.75 mmol) of triethylamine were diluted with 3 mL of dimethylsulfoxide and cooled to 0° C. To the solution was added 72 mg (0.45 mmol) of sulfurtrioxide-pyridine and stirred at room temperature for 4 hours. The reaction solution was distilled in vacuo, diluted with ethyl acetate and washed with water and brine. The organic layer was dried over anhydrous magnesium sulfate (MgSO₄), distilled in vacuo and purified by column chromatography using a mixed solution of dichloromethane and methanol in the ratio of 95:5 to obtain the title compound 50 mg (35%).

¹H NMR (400 MHz, CDCl₃); δ 6.78 (1H, s), 5.23 (2H, s), 5.12 (1H, br), 4.82 (1H, t), 4.26 (4H, m), 4.25 (2H, d), 3.89 (1H, d), 3.37 (1H, t), 2.78 (2H, t), 1.69 (2H, m), 1.47 (9H, s), 0.99 (3H, t)

### Example 1-115

### 4-Amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one

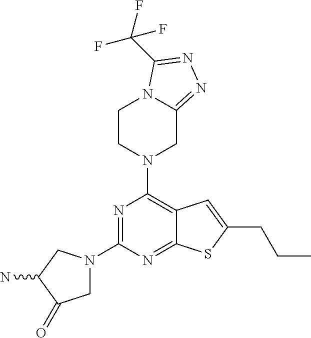

10 mg (0.018 mmol) of the compound obtained from Preparation Example 1-115 was purified according to the same method as Example 1-4 to obtain the title compound 8 mg (89%).

¹H NMR (400 MHz, DMSO, d₆); δ 8.75 (1.2H, br), 8.27 (0.8H, br), 7.27 (1H, d), 5.17 (2H, d), 4.53 (0.6H, t), 4.37 (3H, br), 4.27 (2.4H, br), 3.66 (2H, m), 3.57 (1.3H, m), 3.47 (0.7H, m), 2.79 (2H, m), 1.67 (2H, m), 0.94 (3H, m)

### Example 1-116

### 4-Amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one oxime

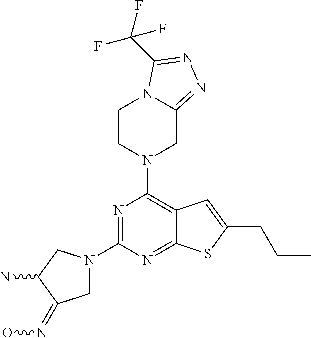

28 mg (0.05 mmol) of the compound obtained from Preparation Example 1-115, 4.1 mg (0.06 mmol) of hydroxyamine; hydrochloride and 3.2 mg (0.03 mmol) of sodium carbonate were diluted with 3 mL of a mixed solution of ethanol and water in the ratio of 2:1, and stirred for 15 hours. The reaction solution was distilled in vacuo, diluted with dichloromethane and washed with water and brine. The organic layer was dried over anhydrous magnesium sulfate (MgSO₄) and distilled in vacuo. The residue was purified by column chromatography using a mixed solution of dichloromethane and methanol in the ratio of 95:5. To a solution of 12 mg of the purified compound in 1 mL of dichloromethane was added 3 mL of 4M HCl/dioxane solution and stirred at room temperature for 30 minutes. The reaction mixture was dried in vacuo to obtain the title compound 9 mg (2-step 32%).

¹H NMR (400 MHz, DMSO, d₆); δ 8.64 (1.5H, br), 7.80 (0.5H, s), 7.55 (0.4H, br), 7.29 (0.6H, br), 5.33 (0.6H, s), 5.18 (1.4H, s), 4.39 (1H, br), 4.31 (3H, m), 4.27 (3H, m), 3.46 (1.4H, m), 3.37 (0.6H, m), 2.90 (0.6H, t), 2.78 (1.4H, t), 1.64 (2H, m), 0.93 (3H, m)

### Example 1-117

### 4-Amino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-one O-methyl-oxime

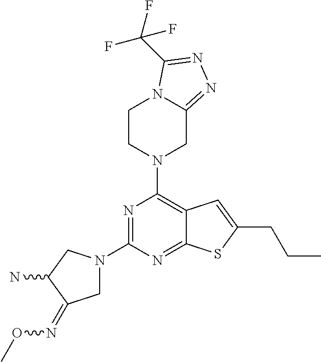

28 mg (0.05 mmol) of the compound obtained from Preparation Example 1-115, 5 mg (0.06 mmol) of methoxyamine; hydrochloride and 3.2 mg (0.03 mmol) of sodium carbonate were diluted with 3 mL of a mixed solution of ethanol and water in the ratio of 2:1, and stirred for 15 hours. The reaction solution was distilled in vacuo, diluted with dichloromethane and washed with water and brine. The organic layer was dried over anhydrous magnesium sulfate (MgSO₄) and distilled in vacuo. The residue was purified by column chromatography using a mixed solution of dichloromethane and methanol in the ratio of 95:5. To a solution of 15 mg of the purified compound in 1 mL of dichloromethane was added 3 mL of 4M HCl/dioxane solution and stirred at room temperature for 30 minutes. The reaction mixture was dried in vacuo to obtain the title compound 14 mg (2-step 49%).

¹H NMR (400 MHz, DMSO, d₆); δ 8.75 (1.5H, br), 7.55 (0.5H, s), 7.35 (0.4H, br), 7.29 (0.6H, br), 5.33 (0.5H, s), 5.18 (1.5H, s), 4.58 (1H, br), 4.38 (3H, m), 4.26 (3H, m), 3.57 (3H, s), 3.47 (1H, m), 2.90 (0.6H, t), 2.80 (1.4H, t), 1.66 (2H, m), 0.93 (3H, m)

### Preparation Example 1-118

### 4-[6-Propyl-4-(3-trifluoromethyl-5,6-dihydro-8H-[1,2,4]triazolo[4,3-a]pyrazin-7-yl)-thieno[2,3-d]pyrimidin-2-yl]-morpholin-2-one

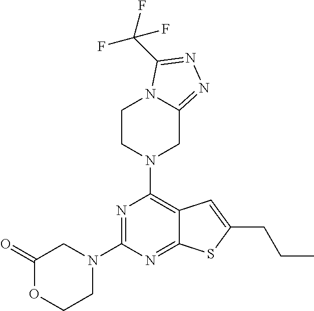

380 mg (0.806 mmol) of the compound obtained from Example 1-34, 28 mg (0.081 mmol) of TPAP and 378 mg (3.220 mmol) of 4-methylmorpholine N-oxide were dissolved in dichloromethane and stirred at room temperature for 5 hours. The reaction mixture was distilled in vacuo and purified by column chromatography using a mixed solution of ethyl acetate and hexane in the ratio of 2:1 to obtain the title compound 130 mg (35%).

¹H NMR (400 MHz, CDCl₃); δ 6.84 (1H, s), 5.23 (2H, s), 4.58 (2H, s), 4.53 (2H, t), 4.36 (2H, m), 4.25 (2H, m), 4.01 (2H, t), 2.80 (2H, t), 1.75 (2H, m), 1.01 (3H, t)

### Example 1-118

### {(2-Hydroxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-acetic acid

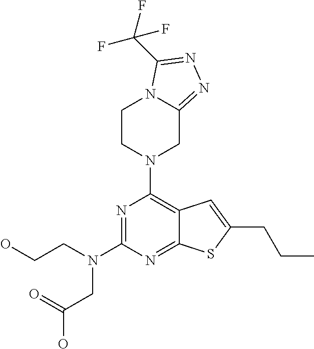

50 mg (0.107 mmol) of the compound obtained from Preparation Example 1-118 was dissolved in a 5:3:1 mixed solution of tetrahydrofuran, water and methanol. To the solution was added 8.98 mg (0.214 mmol) of lithium hydroxide and stirred at room temperature for 3 hours. The reaction mixture was distilled in vacuo and purified by column chromatography using a mixed solution of methanol and dichloromethane in the ratio of 1:5 to obtain the title compound 10 mg (19%).

¹H NMR (400 MHz, CDCl₃); δ 6.94 (1H, s), 5.14 (2H, br), 4.17 (2H, br), 3.92 (4H, br), 3.92 (4H, m), 2.76 (2H, m), 1.71 (2H, m), 1.01 (3H, t)

### Example 1-119

### 2-(2-Amino-thiazol-4-yl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

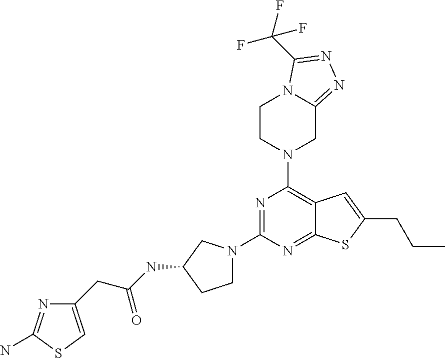

50 mg (0.110 mmol) of the compound obtained from Example 1-14 and 19 mg (0.122 mmol) of (2-aminothiazole-4-yl)acetic acid were dissolved in 5 mL of N,N-dimethylformamide. To the solution was added 63 mg (0.166 mmol) of HBTU and cooled to 0° C. To the reaction mixture was added dropwise 58 uL (0.331 mmol) of diisopropylethylamine and stirred at room temperature for 4 hours. The reaction mixture was diluted with ethyl acetate and washed with water and brine. The organic layer was dried over anhydrous magnesium sulfate (MgSO₄), distilled in vacuo and purified by column chromatography using a mixed solution of methylene chloride and methanol in the ratio of 1:9 to obtain the title compound 28 mg (43%).

¹H NMR (400 MHz, CDCl₃); δ 6.78 (1H, s), 5.18 (2H, m), 4.95 (2H, s), 4.56 (1H, m), 4.32 (2H, m), 4.20 (2H, m), 3.45˜3.49 (3H, m), 2.77 (2H, t), 2.26 (1H, m), 1.94 (1H, m), 1.73 (2H, m), 0.99 (3H, t)

### Example 1-120

### 2-(1H-Imidazol-4-yl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

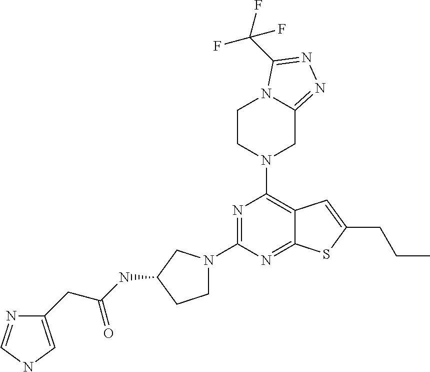

10 mg (16%) of the title compound was obtained according to a method similar to Example 1-119, using 50 mg (0.110 mmol) of the compound obtained from Example 1-14 and 20 mg (0.122 mmol) of (1H-imidazole-4-yl)acetic acid.

¹H NMR (400 MHz, MeOD); δ 7.92 (1H, s), 7.07 (1H, s), 7.00 (1H, s), 5.16 (1H, s), 4.46 (1H, m), 4.38 (2H, m), 4.26 (2H, m), 3.80 (1H, m), 3.64 (2H, m) 3.50 (3H, m), 2.81 (2H, t), 2.24 (1H, m), 2.00 (1H, m), 1.73 (2H, m), 1.00 (3H, t)

### Example 1-121

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-pyridin-2-yl-acetamide

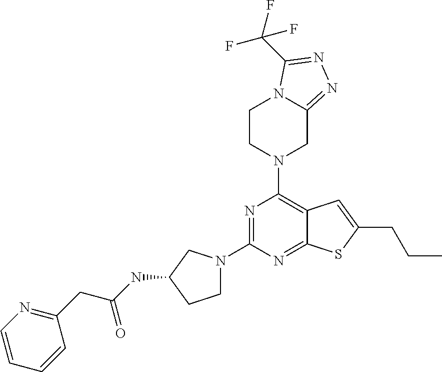

30 mg (47%) of the title compound was obtained according to a method similar to Example 1-119, using 50 mg (0.110 mmol) of the compound obtained from Example 1-14 and 23 mg (0.122 mmol) of pyridine-2-yl-acetic acid hydrochloride.

¹H NMR (400 MHz, CDCl₃); δ 8.46 (1H, d), 7.76 (1H, m), 7.62˜7.67 (1H, m), 7.23 (1H, m), 7.16 (1H, m), 6.77 (1H, s), 5.18 (2H, s), 4.57 (1H, m), 4.32 (2H, m), 4.18 (2H, m), 3.88 (1H, m), 3.71 (2H, s), 3.66 (2H, m), 3.44 (1H, m), 2.77 (2H, m), 2.26 (1H, m), 1.92 (1H, m), 1.71 (2H, m), 0.99 (3H, t)

### Example 1-122

### (2-Ethoxy-ethyl)-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

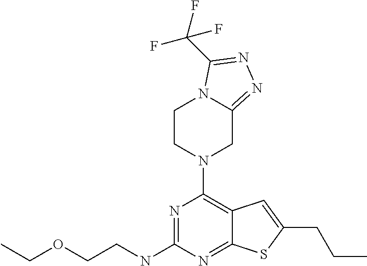

52 mg (92%) of the title compound was obtained according to a method similar to Example 1-1, using 50 mg (0.124 mmol) of the compound obtained from Preparation Example 1-1-3 and 33 mg (0.372 mmol) of 2-ethoxyethylamine.

¹H NMR (400 MHz, CDCl₃); δ 6.79 (1H, s), 5.25 (1H, br s), 5.18 (2H, s), 4.33 (2H, m), 4.19 (2H, m), 3.60 (4H, s), 3.52 (2H, q), 2.77 (2H, t), 1.71 (2H, m), 1.22 (3H, t), 1.00 (3H, t)

### Example 1-123

### 2-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethoxy}-ethanol

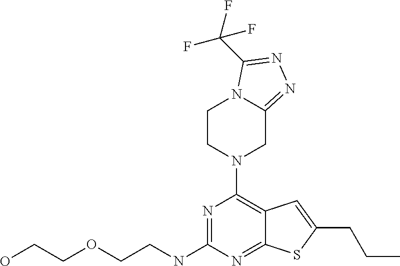

50 mg (85%) of the title compound was obtained according to a method similar to Example 1-1, using 50 mg (0.124 mmol) of the compound obtained from Preparation Example 1-1-3 and 39 mg (0.372 mmol) of 2-(2-aminoethoxy)-ethanol.

¹H NMR (400 MHz, CDCl₃); δ 6.79 (1H, s), 5.29 (1H, m), 5.18 (2H, s), 4.33 (2H, m), 4.18 (2H, m), 3.76 (2H, m), 3.60˜3.70 (6H, m), 2.77 (2H, t), 2.35 (1H, br s), 1.73 (2H, m), 1.00 (3H, t)

### Example 1-124

### 7-[2-(1,1-Dioxo-1lambda*6*-thiomorpholin-4-yl)-6-propyl-thieno[2,3-d]pyrimidin-4-yl]-3-trifluoromethyl-5,6,7,8-tetrahydro-[1,2,4]triazolo[4,3-a]pyrazine

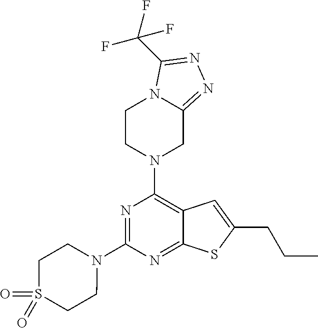

22 mg (35%) of the title compound was obtained according to a method similar to Example 1-1, using 50 mg (0.124 mmol) of the compound obtained from Preparation Example 1-1-3 and 50 mg (0.372 mmol) of thiomorpholine 1,1-dioxide.

¹H NMR (400 MHz, CDCl₃); δ 6.83 (1H, s), 5.18 (2H, s), 4.35 (6H, m), 4.22 (2H, t), 3.04 (4H, m), 2.81 (2H, t), 1.73 (2H, m), 1.01 (3H, t)

### Preparation Example 1-125

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-carbamic acid tert-butyl ester

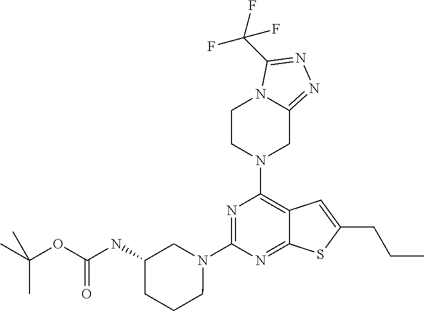

194 mg (69%) of the title compound was obtained according to a method similar to Example 1-1, using 200 mg (0.496 mmol) of the compound obtained from Preparation Example 1-1-3 and 199 mg (0.993 mmol) of (S)-piperidine-3-yl-carbamic acid tert-butyl ester.

¹H NMR (400 MHz, CDCl₃); δ 6.79 (1H, s), 5.19 (2H, m), 4.62 (1H, br), 4.36 (4H, m), 4.20˜4.10 (2H, m), 3.64 (1H, m), 3.46 (1H, m), 3.30 (1H, m), 2.77 (2H, t), 1.93 (1H, m), 1.82˜1.53 (5H, m), 1.45 (9H, s), 1.00 (3H, t)

### Example 1-125

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine

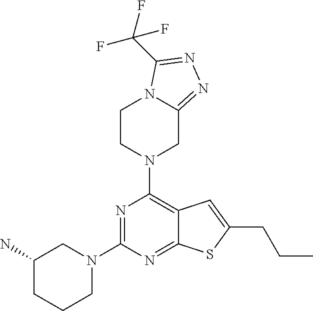

1.2 mg (97%) of the title compound was obtained according to a method similar to Example 1-4, using 1.3 g (2.29 mmol) of the compound obtained from Preparation Example 1-125.

¹H NMR (400 MHz, DMSO, d₆); δ 8.23 (3H, br s), 7.26 (1H, s), 5.17 (2H, m), 4.55 (1H, d), 4.36 (2H, m), 4.25 (3H, m), 3.17˜3.29 (3H, m), 2.79 (2H, t), 2.01 (1H, m), 1.76 (1H, m), 1.64˜1.69 (3H, m), 1.51 (1H, m), 0.94 (3H, t)

### Preparation Example 1-126

### (S)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidine-1-carboxylic acid tert-butyl ester

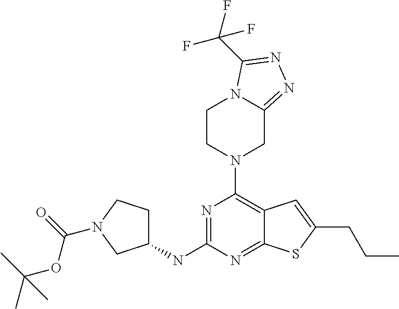

1.9 g (63%) of the title compound was obtained according to a method similar to Example 1-45, using 2.2 g (5.46 mmol) of the compound obtained from Preparation Example 1-1-3 and 2.0 g (10.95 mmol) of (S)-3-amino-pyrrolidin-1-carboxylic acid tert-butyl ester.

¹H NMR (400 MHz, CDCl₃); δ 6.80 (1H, s), 5.20 (2H, m), 4.92 (1H, d), 4.52 (1H, m), 4.32 (2H, m), 4.20 (2H, m), 3.70 (1H, m), 3.47 (2H, m), 3.22˜3.34 (1H, m), 2.78 (2H, t), 2.21 (1H, m), 1.85 (1H, m), 1.46 (9H, s), 1.00 (3H, t)

### Example 1-126

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(S)-pyrrolidin-3-yl-amine

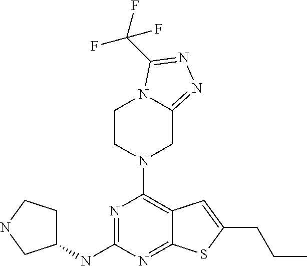

1.7 g (92%) of the title compound was obtained according to a method similar to Example 1-4, using 1.9 g (3.44 mmol) of the compound obtained from Preparation Example 1-126.

¹H NMR (400 MHz, DMSO, d₆); δ 9.26 (3H, br s), 7.65 (1H, br), 7.30 (1H, s), 5.19 (2H, s), 4.54 (1H, m), 4.35 (2H, m), 4.26 (2H, m), 3.45 (1H, m), 3.32 (1H, m), 3.25 (1H, m), 3.11 (1H, m), 2.77 (2H, t), 2.19 (1H, m), 1.99 (1H, m), 1.67 (2H, m), 0.94 (3H, t)

### Preparation Example 1-127-1

### Piperidine-1,4-dicarboxylic acid 1-tert-butyl ester 4-ethyl ester

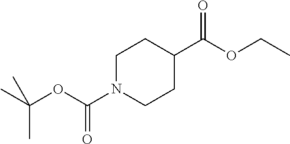

2.38 g (15.14 mmol) of piperidine-4-carboxylic acid ethyl ester was dissolved in 50 mL of dichloromethane. To the solution was added 3.30 g (15.14 mmol) of di tert-butyl dicarbonate and stirred at room temperature for 4 hours. The reaction mixture was distilled in vacuo to obtain the title compound 4.0 g (102%).

¹H NMR (400 MHz, CDCl₃); δ 4.14 (2H, q), 4.00 (2H, m), 2.83 (2H, t), 2.43 (1H, m), 1.89 (2H, m), 1.64 (2H, m), 1.45 (9H, s), 1.25 (3H, t)

### Preparation Example 1-127-2

### Piperidine-1,4-dicarboxylic acid mono-tert-butyl ester

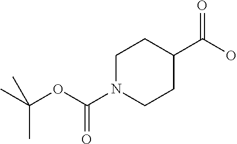

2.8 g (79%) of the title compound was obtained according to a method similar to Example 1-118, using 4.0 g (15.54 mmol) of the compound obtained from Preparation Example 1-127-1 and 978 mg (23.32 mmol) of lithium hydroxide.

### Preparation Example 1-127-3

### C-Phenyl-N-(piperidine-4-carbonyl)-methanesulfonamide; hydrochloride

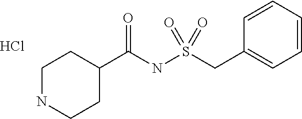

200 mg (0.872 mmol) of the compound obtained from Preparation Example 1-127-2 and 179 mg (1.05 mmol) of phenylmethanesulfonamide were dissolved in 10 mL of dichloromethane. To the solution was added 398 mg (1.05 mmol) of HBTU and cooled to 0° C. To the solution was added dropwise 0.76 mL (4.36 mmol) of diisopropylethylamine and stirred at room temperature for 16 hours. The reaction solution was distilled in vacuo and purified by column chromatography using a mixed solution of dichloromethane and methanol in the ratio of 95:5 to obtain 4-phenylmethanesulfonylaminocarbonyl-piperidine-1-carboxylic acid tert-butyl ester 400 mg (7637-20).

400 mg of the above-obtained compound was dissolved in 5 mL of 4.0 M HCl/dioxane solution and stirred for 1 hour. The reaction mixture was distilled in vacuo to remove a solvent, solidified and cleaned with diethyl ether to obtain the title compound 230 mg (2-step 83%).

¹H NMR (400 MHz, DMSO, d₆); δ 11.69 (1H, s), 8.82 (1H, br), 8.56 (1H, br), 7.39 (3H, m), 7.28 (2H, m), 4.71 (2H, s), 3.26 (2H, m), 2.83 (2H, m), 2.51 (1H, m), 1.85 (2H, m), 1.75 (2H, m)

### Example 1-127

### C-Phenyl-N-{1-[6-propyl-4-(3-trifluoromethyl-5,6-dihydro-8H-[1,2,4]triazolo[4,3-a]pyrazin-7-yl)-thieno[2,3-d]pyrimidin-2-yl]-piperidine-4-carbonyl}-methanesulfonamide

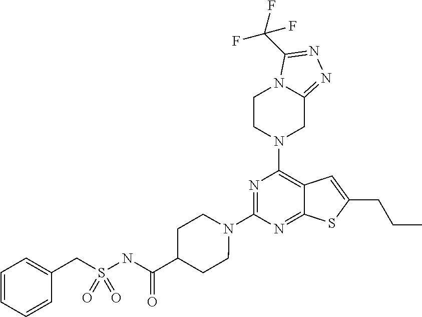

50 mg (62%) of the title compound was obtained according to a method similar to Example 1-1, using 50 mg (0.124 mmol) of the compound obtained from Preparation Example 1-1-3 and 77.66 mg (0.248 mmol) of the compound obtained from Preparation Example 1-127-3.

¹H NMR (400 MHz, CDCl₃); δ 7.59 (1H, m), 7.35˜7.39 (5H, m), 5.15 (2H, s), 4.71 (2H, d), 4.66 (2H, s), 4.33 (2H, m), 4.19 (2H, m), 2.90 (2H, m), 2.78 (2H, t), 2.38 (1H, m), 1.80 (2H, m), 1.65˜1.72 (4H, m), 1.00 (3H, t)

### Preparation Example 1-128-1

### [2-(4-Oxo-thiazolidin-3-yl)-ethyl]-carbamic acid tert-butyl ester

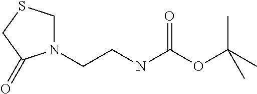

100 mg (0.624 mmol) of (2-aminoethyl)-carbamic acid tert-butyl ester, 37 mg (1.250 mmol) of mercaptoacetic acid and 0.13 mL (1.87 mmol) of para-formaldehyde were dissolved in 10 mL of toluene and stirred at room temperature for 24 hours. The reaction mixture was distilled in vacuo, dissolved in 50 mL of ethyl acetate and washed with water. The organic layer was dried over anhydrous magnesium sulfate (MgSO₄), distilled in vacuo and purified by column chromatography using ethyl acetate to obtain the title compound 77 mg (50%).

¹H NMR (400 MHz, CDCl₃); δ 4.80 (1H, br s), 4.47 (2H, s), 3.55 (2H, s), 3.48 (2H, m), 3.32 (2H, m), 1.44 (9H, s)

### Preparation Example 1-128-2

### 3-(2-Amino-ethyl)-thiazolidin-4-one; hydrochloride

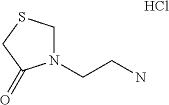

57 mg (100%) of the title compound was obtained according to a method similar to Example 1-4, using 77 mg (0.313 mmol) of the compound obtained from Preparation Example 1-128-1.

¹H NMR (400 MHz, MeOD); δ 4.52 (2H, s), 3.66 (2H, t), 3.58 (2H, s), 3.15 (2H, t)

### Example 1-128

### 3-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-thiazolidin-4-one

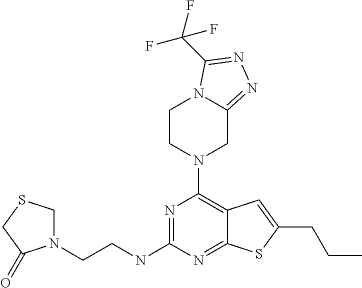

100 mg (77%) of the title compound was obtained according to a method similar to Example 1-1, using 145 mg (0.236 mmol) of the compound obtained from Preparation Example 1-128-2.

¹H NMR (400 MHz, CDCl₃); δ 6.81 (1H, s), 5.20 (2H, s), 5.15 (1H, br), 4.48 (2H, s), 4.32 (2H, m), 4.24 (2H, m), 3.64 (4H, m), 3.52 (2H, s), 2.78 (2H, t), 1.73 (2H, m), 1.00 (3H, t)

### Example 1-129

### (R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-ylamine

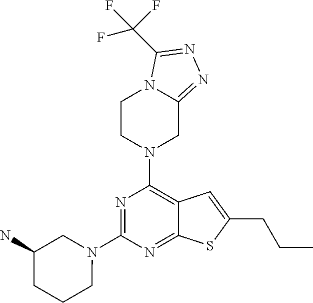

The compound (1.20 g, 2.98 mmol) obtained from Preparation Example 1-1-3, (R)-piperidin-3-ylamine (1.03 g, 5.96 mmol) and diisopropylethylamine (1.926 g, 14.88 mmol) were diluted in butanol (24 mL), and then heated to 150° C. in a microwave reactor and stirred for 1 hour. After distillation of the resulting solution under reduced pressure, dichloromethane (30 mL) and di-t-butyl dicarbonate (0.97 g, 4.46 mmol) were added to the reactant and distilled under reduced pressure to terminate the reaction. The resulting mixture was purified by column chromatography using the 4:1 mixture of dichloromethane and ethyl acetate. The purified compound (1.50 g) was dissolved in dichloromethane (20 mL) and 4M HCl/dioxane solution (10 mL) was added thereto. The resulting solution was stirred at room temperature for 30 minutes and then dried under reduced pressure to give 1.21 g of title compound (step 2; 75%).

¹H NMR (400 MHz, DMSO, d₆); δ 8.43 (2H, br), 7.31 (1H, s), 5.22 (2H, s), 4.58 (1H, d), 4.37 (2H, d), 4.32 (2H, d), 4.22 (1H, d), 3.36 (1H, m), 3.27 (1H, br), 3.17 (1H, br), 2.78 (2H, t), 2.03 (1H, br), 1.78 (1H, br), 1.68 (2H, m), 1.62 (1H, m), 1.47 (1H, br), 0.93 (3H, t)

### Example 1-130

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(R)-pyrrolidin-3-yl-amine

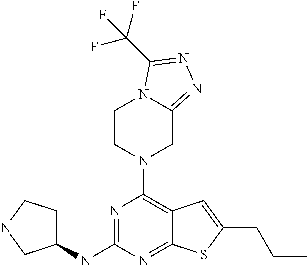

The reaction of the compound (1.40 g, 3.48 mmol) obtained from Preparation Example 1-1-3 and (R)-3-amino-pyrrolidin-1-carboxylic acid t-butyl ester (0.97 g, 5.21 mmol) was carried out according to the similar method with Example 1-45. The resulting mixture was purified by column chromatography using the 1:1 mixture of hexane and ethyl acetate. The purified compound (0.85 g) was dissolved in dichloromethane (20 mL), and 4M HCl/dioxane solution (10 mL) was added thereto. The solution was stirred at room temperature for 30 minutes and then dried under reduced pressure to give 0.50 g of title compound (step 2; 27%).

¹H NMR (400 MHz, DMSO, d₆); δ 7.27 (1H, s), 5.17 (2H, s), 4.52 (2H, br), 4.35 (2H, d), 4.25 (2H, br), 3.44 (1H, m), 3.34 (1H, m), 3.30 (1H, m), 3.11 (1H, m), 2.77 (2H, t), 2.17 (1H, m), 1.97 (1H, m), 1.63 (2H, m), 0.93 (3H, t)

### Example 1-131

### 2-[6-Propyl-4-(3-trifluoromethyl-5,6-dihydro-8H-[1,2,4]triazolo[4,3-a]pyrazin-7-yl)thieno[2,3-d]pyrimidin-2-ylamino]thiazole-4-carboxylic acid ethyl ester

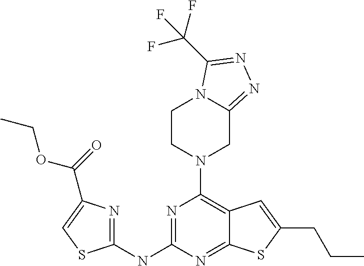

Palladium acetate (II) (5.21 mg, 0.023 mmol), BINAP 17.3 mg (0.028 mmol) and cesium carbonate (113.3 mg, 0.35 mmol) were added to the compound (93.4 mg, 0.23 mmol) obtained from Preparation Example 1-1-3 and 2-aminothiazole-4-carboxylic acid ethyl ester (47.9 mg, 0.28 mmol). The resulting mixture was diluted in toluene (9 mL) and then stirred for 16 hours with reflux. The resulting solution was cooled to room temperature, filtrated by using cellite and then solvent was removed by distillation under reduced pressure. The precipitant obtained by the addition of methanol was filtrated and then dried to give 30 mg of title compound (24%).

¹H NMR (400 MHz, CDCl₃); δ 8.47 (1H, s), 7.77 (1H, s), 6.94 (1H, s), 5.35 (2H, s), 4.43 (2H, d), 4.40 (2H, d), 4.36 (2H, q), 2.84 (2H, t), 1.74 (2H, m), 1.40 (3H, t), 1.00 (3H, t)

### Example 1-132

### 2-[6-Propyl-4-(3-trifluoromethyl-5,6-dihydro-8H-[1,2,4]triazolo[4,3-a]pyrazin-7-yl)thieno[2,3-d]pyrimidin-2-ylamino]thiazole-4-carboxylic acid

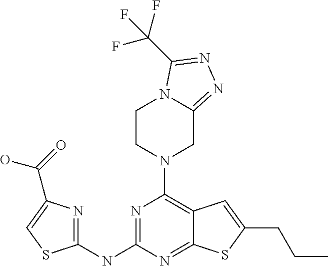

The compound (25.4 mg, 0.047 mmol) obtained from Example 1-131 was dissolved in tetrahydrofuran (1.5 mL), methanol (1 mL) and water (0.5 mL). Lithum hydroxide (3.0 mg, 0.071 mmol) was added thereto, and then reacted at room temperature for 4 hours. The resulting mixture was acidified (pH=3) with 1N HCl aqueous solution, distilled under reduced pressure and then diluted with ethyl acetate. The precipitant obtained by washing with water was filtrated and then dried to give 13.8 mg of title compound (57%).

¹H NMR (500 MHz, MeOD); δ 7.75 (1H, s); δ 7.22 (1H, s), 5.34 (2H, s), 4.49 (2H, d), 4.39 (2H, d), 2.88 (2H, t), 1.74 (2H, m), 1.00 (3H, t)

### Preparation Example 1-133

### 1,2,3,4-Tetrahydro-pyrido[3,4-b]pyrazine

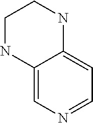

The title compound was prepared according to the known method (see Synthesis (8), 1185-1196, 2007).

### Example 1-133

### 6-[6-Propyl-4-(3-trifluoromethyl-5,6-dihydro-8H-[1,2,4]triazolo[4,3-a]pyrazin-7-yl)thieno[2,3-d]pyrimidin-2-yl]-2,3,4,6-tetrahydro-pyrido[3,4-b]pyrazine

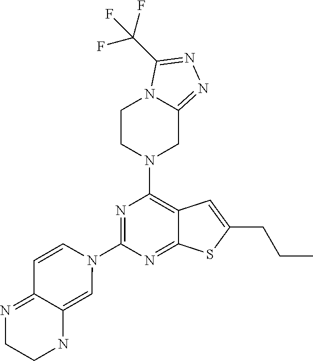

The compound (40.5 mg, 0.1 mmol) obtained from Preparation Example 1-1-3 and the compound (27.2 mg, 0.2 mmol) obtained from Preparation Example 1-133 were dissolved in butanol (2 mL), heated to 150° C. in a microwave reactor, and then the reaction was carried out for 2 hours. The resulting solution was cooled to room tem-perature and distilled under reduced pressure. The resulting mixture was purified by column chromatography using the 1:1 mixture of hexane and ethyl acetate to give 16 mg of title compound (32%).

¹H NMR (500 MHz, CDCl₃); δ 9.96 (1H, br), 9.38 (1H, s), 8.69 (1H, d), 7.19 (1H, d), 7.12 (1H, s), 5.42 (2H, s), 4.68 (2H, d), 4.54 (2H, d), 4.10 (2H, t), 3.28 (2H, t), 2.86 (2H, t), 1.73 (2H, m), 0.98 (3H, t)

### Example 1-134

### 1-[6-Propyl-4-(3-trifluoromethyl-5,6-dihydro-8H-[1,2,4]triazolo[4,3-a]pyrazin-7-yl)thieno[2,3-d]pyrimidin-2-yl]-1,2,3,4-tetrahydro-pyrido[2,3-b]pyrazine

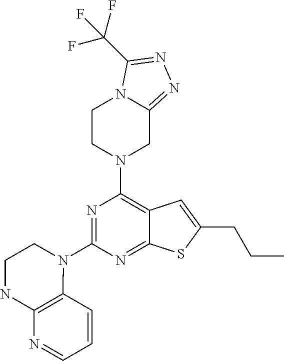

The compound (48.3 mg, 0.12 mmol) obtained from Preparation Example 1-1-3 and 1,2,3,4-tetrahydro-pyrido[2,3-b]pyrazine (32.4 mg, 0.24 mmol) were dissolved in butanol (2 mL), and then heated to 150° C. in a microwave reactor and then the reaction was carried out for 2 hours. The resulting solution was cooled to room temperature and distilled under reduced pressure. The resulting mixture was purified by column chromatography using the 95:5 mixture of dichloromethane and methanol to give 2.5 mg of title compound (4%).

¹H NMR (500 MHz, CDCl₃); δ 10.38 (1H, br), 7.54 (1H, d), 7.13 (1H, s), 6.96 (1H, d), 6.60 (1H, t), 5.43 (2H, s), 4.54 (4H, br), 3.89 (2H, br), 3.44 (2H, br), 2.90 (2H, t), 1.77 (2H, m), 1.00 (3H, t)

### Preparation Example 1-135-1

### 3,4-Dihydro-2H-pyrido[3,4-b]pyrazine-6-carboxylic acid tert-butyl ester

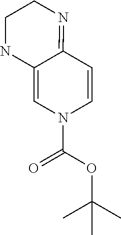

1,2,3,4-tetrahydro-pyrido[3,4-b]pyrazine (48.2 mg, 0.36 mmol) was dissolved in dichloromethane, and then t-butyl dicarbonate (85.6 mg, 0.39 mmol) was added dropwise thereto and stirred at room temperature for 16 hours. The resulting mixture was distilled under reduced pressure and then purified by column chromatography using the 90:10 mixture of dichloromethane and methanol to give 54.2 mg of title compound (65%).

Mass: M+H 235

### Preparation Example 1-135-2

### 6-tert-Butoxycarbonyl-4-[6-propyl-4-(3-trifluoromethyl-5,6-dihydro-8H-[1,2,4]triazolo[4,3-a]pyrazin-7-yl)thieno[2,3-d]pyrimidin-2-yl]-1,2,3,4-tetrahydro-pyrido[3,4-b]pyrazin-6-ium

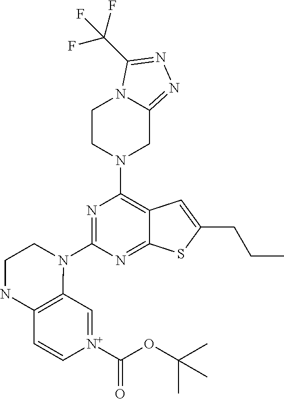

Palladium acetate (II) (4.3 mg, 0.02 mmol), BINAP (14.3 mg, 0.023 mmol) and cesium carbonate (93.8 mg, 0.29 mmol) were added to the compound (77.3 mg, 0.19 mmol) obtained from Preparation Example 1-1-3 and the compound (54.2 mg, 0.23 mmol) obtained from Preparation Example 1-135-1. The resulting mixture was diluted in toluene (6 mL) and then stirred for 16 hours with reflux. The resulting solution was cooled to room temperature, filtrated by using cellite, distilled under reduced pressure to remove solvent and then purified by column chromatography using the 95:5 mixture of dichloromethane and methanol to give 26.5 mg of title compound (23%).

¹H NMR (500 MHz, CDCl₃); δ 8.93 (1H, s), 8.17 (1H, d), 8.09 (1H, d), 6.89 (1H, s), 5.22 (2H, s), 4.18 (6H, m), 3.88 (2H, t), 2.79 (2H, t), 1.72 (2H, m), 1.53 (9H, s), 0.98 (3H, t)

### Example 1-135

### 4-[6-Propyl-4-(3-trifluoromethyl-5,6-dihydro-8H-[1,2,4]triazolo[4,3-a]pyrazin-7-yl)thieno[2,3-d]pyrimidin-2-yl]-1,2,3,4-tetrahydro-pyrido[2,3-b]pyrazine

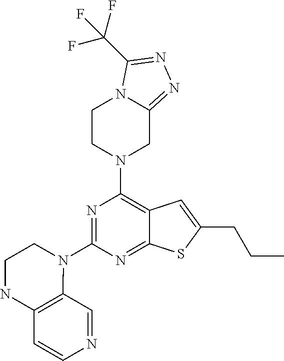

The compound (26.5 mg, 0.044 mmol) obtained from Preparation Example 1-135-2 was cooled to 0° C. 4N HCl/dioxane solution (4 mL) was added dropwise thereto and then stirred at room temperature for 2 hours. The resulting mixture was distilled under reduced pressure to give 23 mg of title compound (99%) without further purification process.

¹H NMR (400 MHz, MeOD); δ 8.91 (1H, s), 7.90 (1H, d), 7.24 (1H, s), 6.92 (1H, d), 5.21 (2H, s), 4.44 (2H, d), 4.36 (2H, d), 3.68 (2H, t), 3.60 (2H, t), 2.89 (2H, t), 1.78 (2H, m), 1.03 (3H, t)

### Preparation Example 2-1-1

### 2,4-Dichloro-6-methyl-thieno[2,3-d]pyrimidine

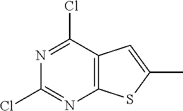

The title compound was prepared according to the known method (see WO 2006/079916).

### Preparation Example 2-1-2

### 7-(2-Chloro-6-methyl-thieno[2,3-d]pyrimidin-4-yl)-3-trifluoromethyl-5,6,7,8-tetrahydro-[1,2,4]triazolo[4,3-a]pyrazine

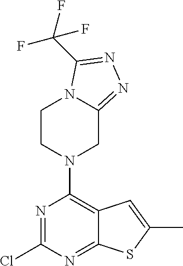

65 mg of the title compound (87%) was prepared according to the same method as Preparation Example 1-1-3 except that the compound (44 mg, 0.2 mmol) obtained from Preparation Example 2-1-1 was used instead of the compound obtained from Preparation Example 1-1-1.

¹H NMR (400 MHz, CDCl₃); δ 7.04 (1H, s), 5.35 (2H, s), 4.38 (4H, m), 2.61 (3H, s)

### Example 2-1

### 4-[6-Methyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

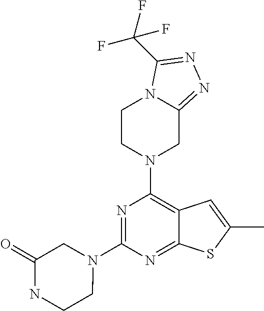

The compound (65 mg, 0.173 mmol) obtained from Preparation Example 2-1-2 and piperazin-2-one (35 mg, 0.346 mmol) were diluted in butanol (2 mL), and then heated to 150° C. in a microwave reactor and stirred for 2 hours. The resulting solution was cooled to room temperature and distilled under reduced pressure, and then diluted with dichloromethane and washed with water. The organic layer was dried with anhydrous magnesium sulfate, distilled under reduced pressure and then purified by column chromatography using the 92:8 mixture of dichloromethane and methanol to give 49 mg of title compound (64%).

¹H NMR (400 MHz, CDCl₃); δ 6.84 (1H, s), 5.21 (2H, s), 4.40 (2H, s), 4.36 (2H, t), 4.25 (2H, t), 3.46 (2H, t), 3.46 (3H, s)

### Preparation Example 2-2-1

### 2,4-Dichloro-6-ethyl-thieno[2,3-d]pyrimidine

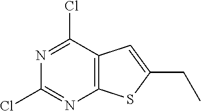

The title compound was prepared according to the known method (see WO 2006/079916).

### Preparation Example 2-2-2

### 7-(2-Chloro-6-ethyl-thieno[2,3-d]pyrimidin-4-yl)-3-trifluoromethyl-5,6,7,8-tetrahydro-[1,2,4]triazolo[4,3-a]pyrazine

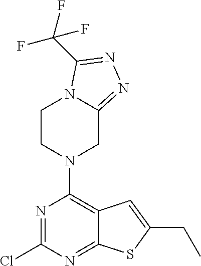

380 mg of the title compound (98%) was prepared according to the same method as Preparation Example 1-1-3 except that the compound (233 mg, 1.0 mmol) obtained from Preparation Example 2-2-1 was used instead of the compound obtained from Preparation Example 1-1-1.

¹H NMR (500 MHz, CDCl₃); δ 7.03 (1H, s), 5.36 (2H, s), 4.38 (4H, m), 2.94 (2H, q), 1.54 (3H, t)

### Example 2-2

### 4-[6-Ethyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

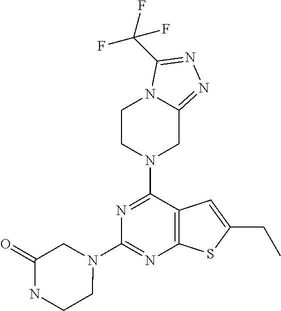

The compound (78 mg, 0.2 mmol) obtained from Preparation Example 2-2-2 and piperazin-2-one (40 mg, 0.4 mmol) were diluted in butanol (2 mL), and then heated to 150° C. in a microwave reactor and stirred for 2 hours. The resulting solution was cooled to room temperature and distilled under reduced pressure, and then diluted with dichloromethane and washed with water. The organic layer was dried with anhydrous magnesium sulfate, distilled under reduced pressure and then purified by column chromatography using the 95:5 mixture of dichloromethane and methanol to give 38 mg of title compound (41%).

¹H NMR (400 MHz, CDCl₃); δ 6.83 (1H, s), 6.18 (1H, s), 5.23 (2H, s), 4.43 (2H, s), 4.35 (2H, t), 4.24 (2H, t), 45 (2H, t), 3.48 (2H, m), 2.85 (2H, q), 1.35 (3H, t)

### Example 2-3

### 3-[6-Ethyl-4-(3-trifluoromethyl-5,6-dihydro-8H-[1,2,4]triazolo[4,3-a]pyrazin-7-yl)-thieno[2,3-d]pyrimidin-2-ylamino]-propane-1,2-diol

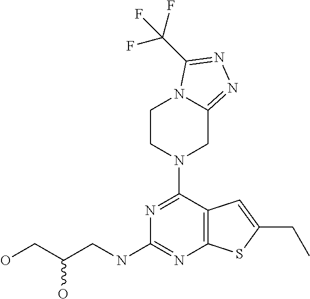

6 mg of the title compound (27%) was prepared according to the same method as Example 2-2 except that 3-amino-1,2-propanediol (9 mg, 0.1 mmol) was used instead of piperazin-2-one.

¹H NMR (400 MHz, CDCl₃); δ 6.80 (1H, s), 5.35 (1H, t), 5.19 (2H, s), 4.32 (2H, t), 4.21 (2H, t), 3.85 (1H, m), 3.70˜3.50 (4H, m), 2.84 (2H, q), 1.33 (3H, t)

### Preparation Example 2-4-1

### 2-{(3aR,4S,6R,6aS)-2,2-Dimethyl-6-[6-ethyl-4-(3-trifluoromethyl-5,6-dihydro-8H-[1,2,4]triazolo[4,3-a]pyrazin-7-yl)-thieno[2,3-d]pyrimidin-2-ylmethyl]-tetrahydro-cyclopenta[1,3]dioxol-4-yloxy}-ethanol

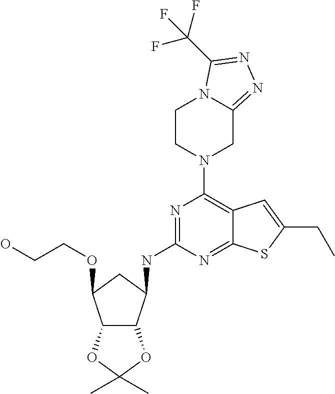

11 mg of the title compound (28%) was prepared according to the same method as Example 2-2 except that the compound (30 mg, 0.14 mmol) obtained from Preparation Example 1-33-1 was used instead of piperazin-2-one.

¹H NMR (400 MHz, CDCl₃); δ 6.79 (1H, s), 5.83 (1H, d), 5.18 (2H, dd), 4.62 (1H, d), 4.53 (1H, d), 4.48˜4.36 (4H, m), 4.09 (1H, m), 3.95 (1H, d), 3.80 (2H, t), 3.73˜3.59 (2H, m), 2.82 (2H, q), 2.27 (1H, m), 1.91 (1H, d), 1.46 (3H, s), 1.33 (3H, t), 1.26 (3H, s)

### Example 2-4

### (1S,2S,3R,5S)-3-[6-Ethyl-4-(3-trifluoromethyl-5,6-dihydro-8H-[1,2,4]triazolo[4,3-a]pyrazin-7-yl)-thieno[2,3-d]pyrimidin-2-ylamino]-5-(2-hydroxy-ethoxy)-cyclopentane-1,2-diol

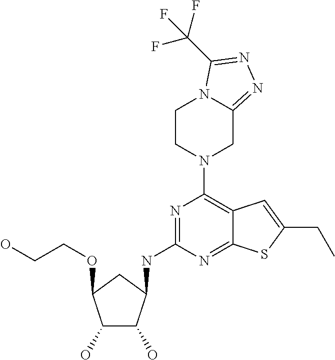

8 mg of the title compound (80%) was prepared according to the same method as Example 1-33 except that the compound (11 mg, 0.019 mmol) obtained from Preparation Example 2-4-1 was used instead of the compound obtained from Preparation Example 1-33-2.

¹H NMR (400 MHz, CDCl₃); δ 6.81 (1H, s), 5.19 (2H, dd), 4.32 (2H, t), 4.22 (2H, t), 4.09 (2H, m), 4.03 (1H, t), 3.91 (1H, t), 3.75 (2H, t), 3.73˜3.60 (2H, m), 2.83 (2H, q), 2.67 (1H, m), 1.61 (1H, m), 1.33 (3H, t)

### Example 2-5

### 2-[6-Ethyl-4-(3-trifluoromethyl-5,6-dihydro-8H-[1,2,4]triazolo[4,3-a]pyrazin-7-yl)-thieno[2,3-d]pyrimidin-2-ylamino]-ethanol

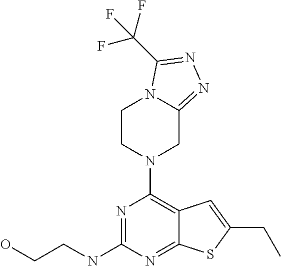

27 mg of the title compound (66%) was prepared according to the same method as Example 2-2 except that ethanolamine (12 mg, 0.2 mmol) was used instead of piperazin-2-one.

¹H NMR (400 MHz, CDCl₃); δ 6.81 (1H, s), 5.18 (2H, s), 4.34 (2H, t), 4.22 (2H, t), 3.77 (2H, t), 3.56 (2H, t), 2.84 (2H, q), 1.34 (3H, t)

### Example 2-6

### 2-[[6-Ethyl-4-(3-trifluoromethyl-5,6-dihydro-8H-[1,2,4]triazolo[4,3-a]pyrazin-7-yl)-thieno[2,3-d]pyrimidin-2-yl]-(2-hydroxy-ethyl)-amino]-ethanol

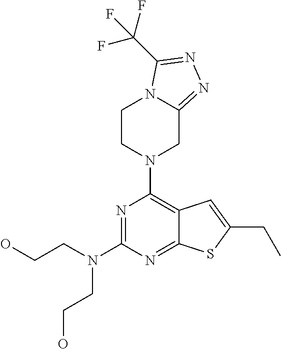

31 mg of the title compound (67%) was prepared according to the same method as Example 2-2 except that diethanolamine (21 mg, 0.2 mmol) was used instead of piperazin-2-one.

¹H NMR (400 MHz, CDCl₃); δ 6.78 (1H, s), 5.13 (2H, s), 4.31 (2H, t), 4.20 (2H, t), 3.89 (4H, t), 3.80 (4H, t), 2.83 (2H, q), 1.33 (3H, t)

### Preparation Example 2-7-1

### 4-[6-Ethyl-4-(3-trifluoromethyl-5,6-dihydro-8H-[1,2,4]triazolo[4,3-a]pyrazin-7-yl)-thieno[2,3-d]pyrimidin-2-yl]-piperazine-1-carboxylic acid tert-butyl ester

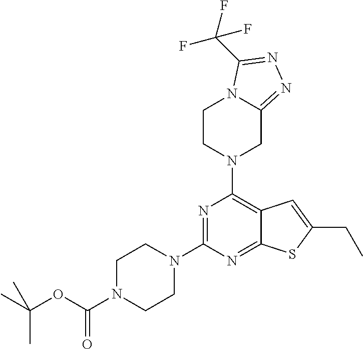

31 mg of the title compound (56%) was prepared according to the same method as Example 2-2 except that piperazin-1-carboxylic acid t-butyl ester (37 mg, 0.2 mmol) was used instead of piperazin-2-one.

¹H NMR (400 MHz, CDCl₃); δ 6.79 (1H, s), 5.18 (2H, s), 4.34 (2H, t), 4.21 (2H, t), 3.78 (2H, t), 3.50 (2H, t), 2.84 (2H, q), 1.49 (9H, s), 1.34 (3H, t)

### Example 2-7

### 7-(6-Ethyl-2-piperazin-1-yl-thieno[2,3-d]pyrimidin-4-yl)-3-trifluoromethyl-5,6,7,8-tetrahydro-[1,2,4]triazolo[4,3-a]pyrazine

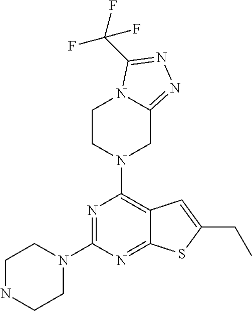

30 mg of the title compound (100%) was prepared according to the same method as Example 1-4 except that the compound (31 mg, 0.058 mmol) obtained from Preparation Example 2-7-1 was used instead of the compound obtained from Preparation Example 1-4-1.

¹H NMR (400 MHz, DMSO, d₆); δ 9.26 (2H, br s), 7.27 (1H, s), 5.16 (2H, s), 4.36 (2H, t), 4.24 (2H, t), 4.15˜3.90 (2H, m), 3.69 (1H, m), 3.48 (1H, m), 3.14 (2H, br s), 2.84 (2H, q), 1.28 (3H, t)

### Preparation Example 2-8-1

### {2-[6-Ethyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-carbamic acid tert-butyl ester

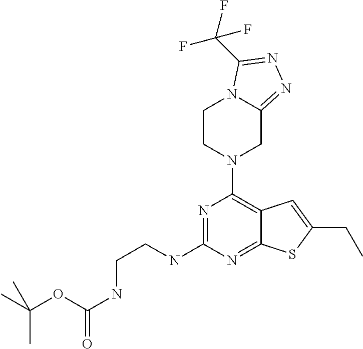

The compound (39 mg, 0.1 mmol) obtained from Preparation Example 2-2-2 and (2-amino-ethyl)-carbamic acid t-butyl ester (32 mg, 0.2 mmol) were diluted in butanol (2 mL), and then heated to 150° C. in a microwave reactor and stirred for 2 hours. The resulting solution was cooled to room temperature, distilled under reduced pressure and dissolved in methanol (5 mL). Di t-butyl dicarbonate (218 mg, 1.0 mmol) was added thereto and stirred for 16 hours. The resulting mixture was distilled under reduced pressure to remove solvent and then purified by column chromatography using the 95:5 mixture of dichloromethane and methanol to give 33 mg of title compound (65%).

¹H NMR (400 MHz, CDCl₃); δ 6.80 (1H, s), 5.25 (1H, t), 5.20 (2H, s), 5.04 (1H, br s), 4.34 (2H, t), 4.23 (2H, t), 3.53 (2H, q), 3.35 (2H, q), 2.83 (2H, q), 1.43 (9H, s), 1.33 (3H, t)

### Example 2-8

### N*1*-[6-Ethyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine

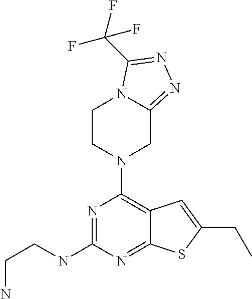

31 mg of the title compound (100%) was prepared according to the same method as Example 1-4 except that the compound (31 mg, 0.058 mmol) obtained from Preparation Example 2-8-1 was used instead of the compound obtained from Preparation Example 1-4-1.

¹H NMR (400 MHz, DMSO, d₆); δ 8.03 (2H, br s), 7.31 (1H, s), 5.23 (2H, s), 4.37 (2H, m), 3.75˜3.43 (4H, m), 3.00 (2H, m), 2.84 (2H, q), 1.28 (3H, t)

### Preparation Example 2-9-1

### [6-Ethyl-4-(3-trifluoromethyl-5,6-dihydro-8H-[1,2,4]triazolo[4,3-a]pyrazin-7-yl)-thieno[2,3-d]pyrimidin-2-ylamino]-acetic acid butyl ester

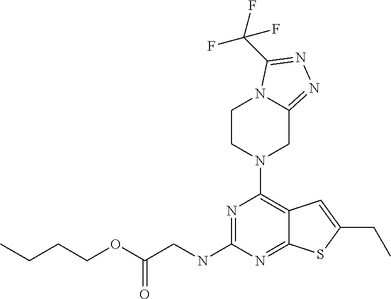

The compound (39 mg, 0.1 mmol) obtained from Preparation Example 2-2-2, hydrochloric acid salt of glycine methyl ester (25 mg, 0.2 mmol) and diisopropylethylamine (26 mg, 0.2 mmol) were diluted in butanol (2 mL), and then heated to 150° C. in a microwave reactor and stirred for 2 hours. The resulting mixture was distilled under reduced pressure to remove solvent and then purified by column chromatography using the 93:7 mixture of dichloromethane and methanol to give 16 mg of title compound (33%).

¹H NMR (400 MHz, CDCl₃); δ 6.81 (1H, s), 5.41 (1H, t), 5.20 (2H, s), 4.33 (2H, t), 4.27˜4.12 (4H, m), 2.84 (2H, q), 1.63 (2H, m), 1.38 (2H, m), 1.34 (3H, t), 0.92 (3H, t)

### Example 2-9

### [6-Ethyl-4-(3-trifluoromethyl-5,6-dihydro-8H-[1,2,4]triazolo[4,3-a]pyrazin-7-yl)-thieno[2,3-d]pyrimidin-2-ylamino]-acetic acid

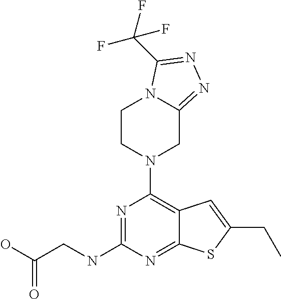

12 mg of the title compound (86%) was prepared according to the same method as Example 1-46 except that the compound (16 mg, 0.033 mmol) obtained from Preparation Example 2-9-1 was used instead of the compound obtained from Example 1-45.

¹H NMR (400 MHz, CD₃OD); δ 7.06 (1H, s), 5.19 (2H, s), 4.42 (2H, t), 4.27 (2H, t), 47 (2H, s), 2.88 (2H, q), 1.35 (3H, t)

### Example 2-10

### 7-[6-Ethyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine

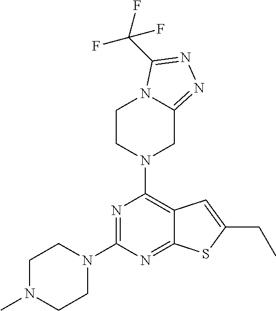

45 mg of the title compound (100%) was prepared according to the same method as Example 2-2 except that 1-methylpiperazin (20 mg, 0.2 mmol) was used instead of piperazin-2-one.

¹H NMR (400 MHz, CDCl₃); δ 6.81 (1H, s), 5.21 (2H, s), 4.38 (2H, t), 4.23 (2H, t), 3.86 (4H, t), 2.87 (2H, q), 2.51 (4H, t), 2.38 (3H, s), 1.37 (3H, t)

### Preparation Example 2-11-1

### 2,4-Dichloro-6-isopropyl-thieno[2,3-d]pyrimidine

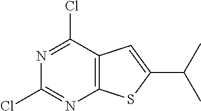

The title compound was prepared according to the known method (see WO 2006/079916).

### Preparation Example 2-11-2

### 7-(2-Chloro-6-isopropyl-thieno[2,3-d]pyrimidin-4-yl)-3-trifluoromethyl-5,6,7,8-tetrahydro-[1,2,4]triazolo[4,3-a]pyrazine

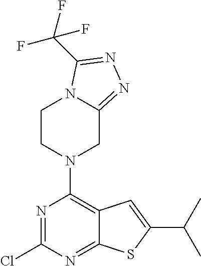

139 mg of the title compound (79%) was prepared according to the similar method with Preparation Example 1-1-3 by using the compound (108 mg, 0.437 mmol) obtained from Preparation Example 2-11-1 and the compound (100 mg, 0.437 mmol) obtained from Preparation Example 1-1-2.

¹H NMR (400 MHz, CDCl₃); δ 7.03 (1H, s), 5.36 (2H, s), 4.37 (4H, m), 3.25 (1H, m), 1.42 (3H, s), 1.40 (3H, s)

### Example 2-11

### 2-[6-Isopropyl-4-(3-trifluoromethyl-5,6-dihydro-8H-[1,2,4]triazolo[4,3-a]pyrazin-7-yl)-thieno[2,3-d]pyrimidin-2-ylamino]-ethanol

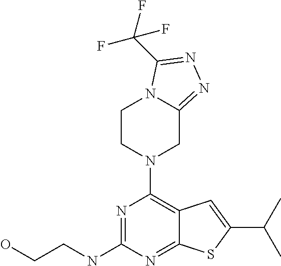

40 mg of the title compound (94%) was prepared according to the similar method with Example 1-1 by using the compound (40 mg, 0.099 mmol) obtained from Preparation Example 2-11-2 and 2-aminoethanol (18 mg, 0.298 mmol).

¹H NMR (500 MHz, CDCl₃); δ 6.78 (1H, s), 5.29 (1H, br s), 5.19 (2H, s), 4.31 (2H, s), 4.19 (2H, t), 3.81 (2H, m), 3.59 (1H, m), 1.35 (3H, s), 1.34 (3H, s)

### Example 2-12

### 4-[6-Isopropyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

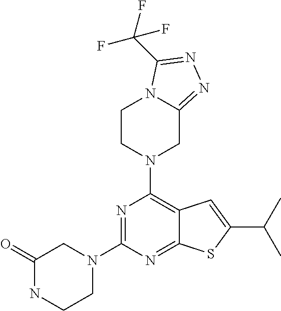

The compound (40 mg, 0.099 mmol) obtained from Preparation Example 2-11-2 and piperazin-2-one (30 mg, 0.298 mmol) were diluted in butanol (3 mL), and then heated to 150° C. in a microwave reactor and stirred for 1 hour. The resulting mixture was distilled under reduced pressure and then purified by column chromatography using the 9:1 mixture of dichloromethane and methanol to give 40 mg of 4-[6-isopropyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one. 40 mg of obtained 4-[6-isopropyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one was dissolved in dichloromethane (2 mL) and then 4M HCl/dioxane solution (3 mL) was added thereto and stirred at room temperature for 30 minutes. The resulting mixture was dried under reduced pressure to give 45 mg of title compound (step 2; 90%).

¹H NMR (500 MHz, DMSO, d₆); δ 8.05 (1H, br s), 7.19 (1H, s), 5.16 (2H, s), 4.37 (2H, m), 4.23 (2H, m), 4.18 (2H, s), 3.89 (2H, m), 3.27 (2H, m), 3.18 (1H, m), 1.32 (3H, s), 1.30 (3H, s)

### Example 2-13

### 7-[6-Isopropyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine

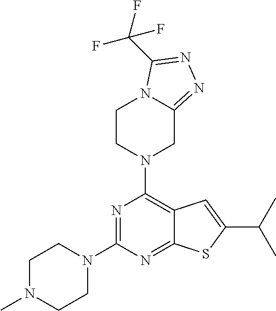

30 mg of the title compound (65%) was prepared according to the similar method with Example 1-1 by using the compound (40 mg, 0.099 mmol) obtained from Preparation Example 2-11-2 and 1-methylpiperazin (30 mg, 0.298 mmol).

¹H NMR (400 MHz, CDCl₃); δ 6.76 (1H, s), 5.17 (1H, br s), 4.34 (2H, t), 4.19 (2H, t), 3.82 (2H, m), 3.13 (1H, m), 2.47 (2H, m), 2.35 (3H, s), 1.36 (3H, s), 1.35 (3H, s)

### Preparation Example 2-14-1

### 6-Butyl-2,4-dichloro-thieno[2,3-d]pyrimidine

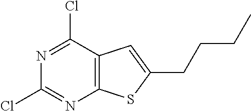

The title compound was prepared according to the known method (see WO 2006/079916).

### Preparation Example 2-14-2

### 7-(6-Butyl-2-chloro-thieno[2,3-d]pyrimidin-4-yl)-3-trifluoromethyl-5,6,7,8-tetrahydro-[1,2,4]triazolo[4,3-a]pyrazine

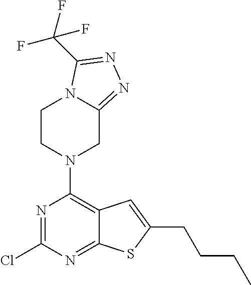

147 mg of the title compound (92%) was prepared according to the similar method with Preparation Example 1-1-3 by using the compound (100 mg, 0.383 mmol) obtained from Preparation Example 2-14-1 and the compound (88 mg, 0.459 mmol) obtained from Preparation Example 1-1-2.

¹H NMR (400 MHz, CDCl₃); δ 7.03 (1H, s), 5.36 (2H, s), 4.38 (4H, m), 2.91 (2H, t), 1.73 (2H, m), 1.44 (2H, m), 0.98 (3H, t)

### Example 2-14

### 4-[6-Butyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

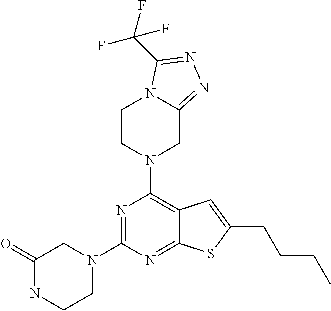

46 mg of the title compound (93%) was prepared according to the similar method with Example 2-12 by using the compound (40 mg, 0.096 mmol) obtained from Preparation Example 2-14-2 and piperazin-2-one (30 mg, 0.288 mmol).

¹H NMR (400 MHz, DMSO, d₆); δ 8.06 (1H, br s), 7.25 (1H, s), 5.15 (2H, s), 4.37 (2H, m), 4.23 (2H, m), 4.18 (2H, s), 3.89 (2H, m), 3.27 (2H, m), 2.81 (2H, t), 1.63 (2H, m), 1.35 (2H, m), 0.92 (3H, t)

### Example 2-15

### 2-[6-Butyl-4-(3-trifluoromethyl-5,6-dihydro-8H-[1,2,4]triazolo[4,3-a]pyrazin-7-yl)-thieno[2,3-d]pyrimidin-2-ylamino]-ethanol

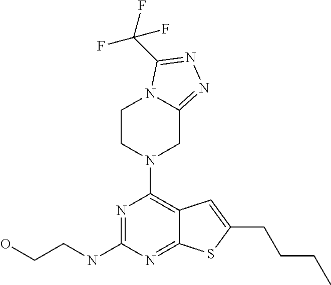

35 mg of the title compound (83%) was prepared according to the similar method with Example 1-1 by using the compound (40 mg, 0.096 mmol) obtained from Preparation Example 2-14-2 and 2-aminoethanol (17 mg, 0.288 mmol).

¹H NMR (400 MHz, CDCl₃); δ6.79 (1H, s), 5.21 (1H, m), 5.19 (2H, s), 4.32 (2H, m), 4.20 (2H, m), 3.83 (2H, m), 3.60 (2H, m), 2.80 (2H, t), 1.68 (2H, m), 1.40 (2H, m), 0.94 (3H, t)

### Example 2-16

### 7-[6-Butyl-2-(4-methyl-piperazin-1-yl)-thieno[2,3-d]pyrimidin-4-yl]-3-trifluoromethyl-5,6,7,8-tetrahydro-[1,2,4]triazolo[4,3-a]pyrazine

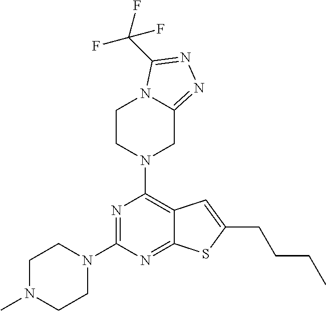

35 mg of the title compound (76%) was prepared according to the similar method with Example 1-1 by using the compound (40 mg, 0.096 mmol) obtained from Preparation Example 2-14-2 and 1-methylpiperazin (29 mg, 0.288 mmol).

¹H NMR (400 MHz, DMSO, d₆); δ 6.75 (1H, s), 5.17 (1H, s), 4.34 (2H, m), 4.19 (2H, m), 3.82 (4H, m), 2.80 (2H, t), 2.48 (4H, m), 2.35 (3H, s), 1.68 (2H, m), 1.42 (2H, m), 0.95 (3H, t)

### Preparation Example 2-17-1

### 2,4-Dichloro-6-isobutyl-thieno[2,3-d]pyrimidine

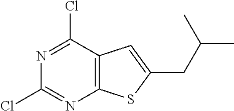

The title compound was prepared according to the known method (see WO 2006/079916).

### Preparation Example 2-17-2

### 7-(2-Chloro-6-isobutyl-thieno[2,3-d]pyrimidin-4-yl)-3-trifluoromethyl-5,6,7,8-tetrahydro-[1,2,4]triazolo[4,3-a]pyrazine

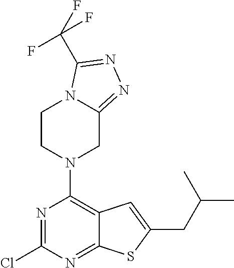

90 mg of the title compound (56%) was prepared according to the similar method with Preparation Example 1-1-3 by using the compound (100 mg, 0.383 mmol) obtained from Preparation Example 2-17-1 and the compound (105 mg, 0.459 mmol) obtained from Preparation Example 1-1-2.

¹H NMR (400 MHz, CDCl₃); δ 7.02 (1H, s), 5.36 (2H, s), 4.39 (4H, m), 2.76 (2H, d), 1.99 (1H, m), 1.01 (3H, s), 0.99 (3H, s)

### Example 2-17

### 4-[6-Isobutyl-4-(3-trifluoromethyl-5,6-dihydro-8H-[1,2,4]triazolo[4,3-a]pyrazin-7-yl)-thieno[2,3-d]pyrimidin-2-yl]-piperazin-2-one

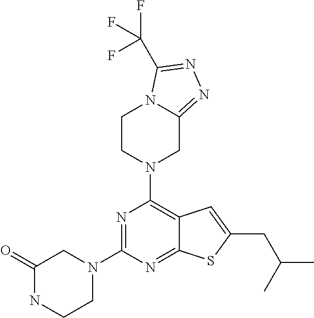

27 mg of the title compound (73%) was prepared according to the similar method with Example 2-12 by using the compound (30 mg, 0.072 mmol) obtained from Preparation Example 2-17-2 and piperazin-2-one (22 mg, 0.216 mmol).

¹H NMR (400 MHz, DMSO, d₆); δ 8.05 (1H, br s), 7.24 (1H, s), 5.15 (2H, s), 4.37 (2H, m), 4.22 (2H, m), 4.18 (2H, s), 3.88 (2H, m), 3.27 (2H, m), 2.67 (2H, d), 1.95 (1H, m), 0.94 (3H, s), 0.92 (3H, s)

### Example 2-18

### (R)-1-[6-Isobutyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ol

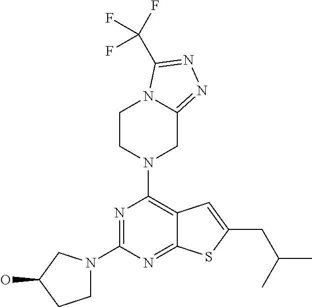

18 mg of the title compound (54%) was prepared according to the similar method with Example 1-1 by using the compound (30 mg, 0.072 mmol) obtained from Preparation Example 2-17-2 and (R)-pyrrolidin-3-ol (27 mg, 0.022 mmol).

¹H NMR (400 MHz, CDCl₃); δ 6.75 (1H, s), 5.18 (2H, s), 4.60 (1H, br 4.33 (2H, m), 4.20 (2H, m), 3.68˜3.74 (4H, m), 2.65 (2H, d), 2.20 (1H, m), 2.15 (1H, m), 1.95 (1H, m), 0.97 (3H, s), 0.96 (3H, s)

### Preparation Example 2-19-1

### 2,4-Dichloro-6-(3,3,3-trifluoropropyl)-thieno[2,3-d]pyrimidine

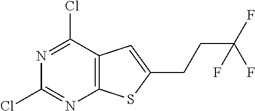

The title compound was prepared according to the known method (see WO 2006/079916).

### Preparation Example 2-19-2

### 7-[2-Chloro-6-(3,3,3-trifluoropropyl)-thieno[2,3-d]pyrimidin-4-yl]-3-trifluoromethyl-5,6,7,8-tetrahydro-[1,2,4]triazolo[4,3-a]pyrazine

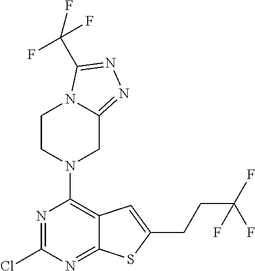

110 mg of the title compound (43%) was prepared according to the similar method with Preparation Example 1-1-3 by using the compound (100 mg, 0.332 mmol) obtained from Preparation Example 2-19-1 and the compound (94 mg, 0.399 mmol) obtained from Preparation Example 1-1-2.

¹H NMR (400 MHz, CDCl₃); δ 7.14 (1H, s), 5.36 (2H, s), 4.40 (4H, s), 3.20 (2H, m), 2.56 (2H, m)

### Example 2-19

### 4-[4-(3-Trifluoromethyl-5,6-dihydro-8H-[1,2,4]triazolo[4,3-a]pyrazin-7-yl)-6-(3,3,3-trifluoro-propyl)-thieno[2,3-d]pyrimidin-2-yl]-piperazin-2-one

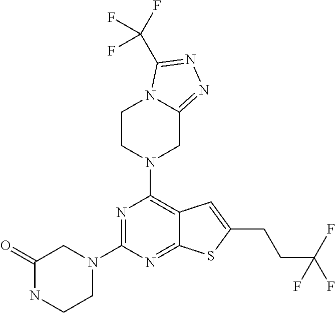

37 mg of the title compound (100%) was prepared according to the similar method with Example 2-12 by using the compound (30 mg, 0.066 mmol) obtained from Preparation Example 2-19-2 and piperazin-2-one (20 mg, 0.197 mmol).

¹H NMR (400 MHz, DMSO, d₆); δ 8.07 (1H, br s), 7.40 (1H, s), 5.17 (2H, s), 4.36 (2H, m), 4.24 (2H, m), 4.19 (2H, s), 3.88 (2H, m), 3.27 (2H, m), 3.06 (2H, m), 2.72 (2H, m)

### Example 2-20

### (R)-1-[4-(3-Trifluoromethyl-5,6-dihydro-8H-[1,2,4]triazolo[4,3-a]pyrazin-7-yl)-6-(3,3,3-trifluoro-propyl)-thieno[2,3-d]pyrimidin-2-yl]-pyrrolidin-3-ol

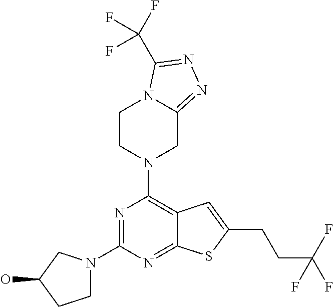

30 mg of the title compound (90%) was prepared according to the similar method with Example 1-1 by using the compound (30 mg, 0.066 mmol) obtained from Preparation Example 2-19-2 and (R)-pyrrolidin-3-ol (24 mg, 0.197 mmol).

¹H NMR (400 MHz, DMSO, d₆); δ 7.30 (1H, s), 5.09 (2H, s), 4.87 (2H, d), 4.32 (3H, m), 4.17 (2H, m), 3.51 (2H, m), 3.01 (2H, m), 2.68 (2H, m), 1.96 (1H, m), 1.84 (1H, m)

### Preparation Example 3-1-1

### 7-[2-(2,2-Dimethyl-[1,3]dioxolan-4-ylmethoxy)-6-propyl-thieno[2,3-d]pyrimidin-4-yl]-3-trifluoromethyl-5,6,7,8-tetrahydro-[1,2,4]triazolo[4,3-a]pyrazine

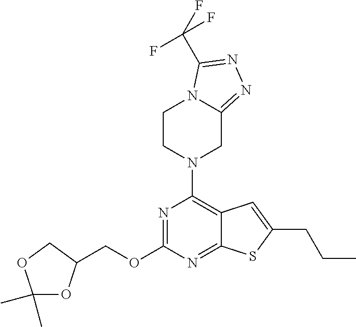

1.03 g of the title compound (83%) was prepared according to the same method as Example 1-45 except that (2,2-dimethyl-[1,3]dioxolan-4-yl)-methanol (0.66 g, 5.0 mmol) was used instead of piperidine-3-carboxylic acid ethyl ester.

¹H NMR (400 MHz, CDCl₃); δ 6.94 (1H, s), 5.30 (2H, s), 4.57˜4.28 (7H, m), 4.17 (1H, dd), 3.91 (1H, dd), 2.84 (2H, t), 1.75 (2H, m), 1.47 (3H, s), 1.39 (3H, s), 1.01 (3H, t)

### Example 3-1

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propane-1,2-diol

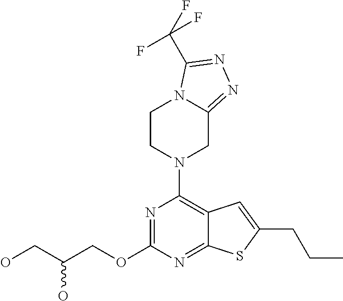

The compound (1.03 g, 2.07 mmol) obtained from Preparation Example 3-1-1 was dissolved in methanol (15 mL), and then concentrated hydrochloric acid aqueous solution (0.8 mL) was added thereto and stirred for 1 hour. The resulting solution was basicified with 10 M NaOH aqueous solution, distilled under reduced pressure, and then diluted with ethyl acetate and washed with water and salt water. The organic layer was dried with anhydrous magnesium sulfate, distilled under reduced pressure and then purified by column chromatography using the 90:10 mixture of dichloromethane and methanol to give 0.89 g of title compound (94%).

¹H NMR (400 MHz, CDCl₃); δ 6.95 (1H, s), 5.30 (2H, s), 4.48 (2H, m), 4.33 (4H, m), 4.12 (1H, m), 3.75 (2H, m), 3.47 (1H, br s), 2.83 (2H, t), 2.72 (1H, br s), 1.74 (2H, m), 1.01 (3H, t)

### Preparation Example 3-2-1

### (2,2-Dimethyl-[1,3]dioxan-5-yl)-methanol

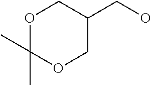

The title compound was prepared according to the known method (see WO 2006/079916).

### Preparation Example 3-2-2

### 7-[2-(2,2-Dimethyl-[1,3]dioxan-5-ylmethoxy)-6-propyl-thieno[2,3-d]pyrimidin-4-yl]-3-trifluoromethyl-5,6,7,8-tetrahydro-[1,2,4]triazolo[4,3-a]pyrazine

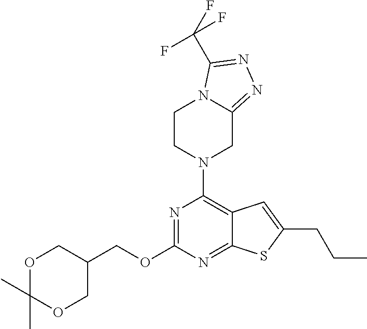

128 mg of the title compound (51%) was prepared according to the same method as Example 1-45 except that the compound (146 mg, 1.0 mmol) obtained from Preparation Example 3-2-1 was used instead of piperidine-3-carboxylic acid ethyl ester.

¹H NMR (500 MHz, CDCl₃); δ 6.93 (1H, s), 5.30 (2H, s), 4.47 (2H, d), 4.34 (4H, m), 4.08 (2H, dd), 3.86 (2H, dd), 2.82 (2H, t), 2.20 (1H, m), 1.74 (2H, m), 1.45 (3H, s), 1.42 (3H, s), 1.00 (3H, t)

### Example 3-2

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-propane-1,3-diol

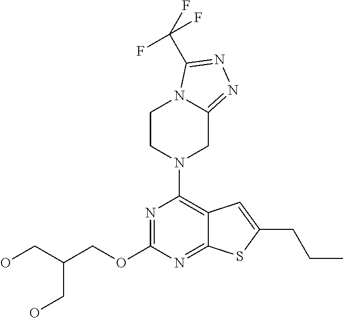

84 mg of the title compound (71%) was prepared according to the same method as Example 3-1 except that the compound (128 mg, 0.25 mmol) obtained from Preparation Example 3-2-2 was used instead of the compound obtained from Preparation Example 3-1-1.

¹H NMR (400 MHz, CDCl₃); δ 6.94 (1H, s), 5.30 (2H, s), 4.56 (2H, d), 4.35 (4H, m), 3.88 (4H, br s), 3.18 (2H, br s), 2.82 (2H, t), 2.23 (1H, m), 1.74 (2H, m), 1.01 (3H, t)

### Preparation Example 3-3-1

### 2-(tert-Butyl-dimethyl-silanyloxy)-ethanol

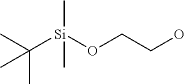

The title compound was prepared according to the known method (see J. Org. Chem., 51, 3388 (1986)).

### Preparation Example 3-3-2

### 7-{2-[2-(t-Butyl-dimethyl-silanyloxy)-ethoxy]-6-propyl-thieno[2,3-d]pyrimidin-4-yl}-3-trifluoromethyl-5,6,7,8-tetrahydro-[1,2,4]triazolo[4,3-a]pyrazine

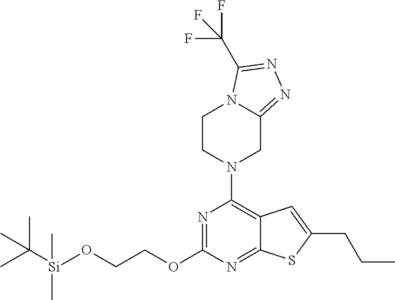

36 mg of the title compound (67%) was prepared according to the same method as Example 1-45 except that the compound (35 mg, 0.2 mmol) obtained from Preparation Example 3-3-1 was used instead of piperidine-3-carboxylic acid ethyl ester.

¹H NMR (500 MHz, CDCl₃); δ 6.92 (1H, s), 5.29 (2H, s), 4.43 (2H, t), 4.36 (2H, m), 4.29 (2H, m), 3.97 (2H, t), 2.81 (2H, t), 1.74 (2H, m), 1.00 (3H, t), 0.87 (9H, s), 0.06 (6H, s)

### Example 3-3

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethanol

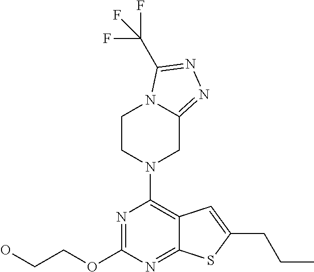

The compound (36 mg, 0.07 mmol) obtained from Preparation Example 3-3-2 was dissolved in tetrahydrofuran (3 mL), and then 1M tetrabutylammonium fluoride (0.25 mL, 0.25 mmol) was added thereto and stirred for 2 hours. Ethyl acetate (20 mL) was added thereto and washed with water (10 mL) twice. The organic layer was dried with anhydrous magnesium sulfate, distilled under reduced pressure and then purified by column chromatography using the 1:1 mixture of hexane and ethyl acetate to give 20 mg of title compound (71%).

¹H NMR (500 MHz, CDCl₃); δ 6.94 (1H, s), 5.31 (2H, s), 4.51 (2H, t), 4.37 (2H, m), 4.32 (2H, m), 3.98 (2H, t), 2.82 (2H, t), 1.75 (2H, m), 1.00 (3H, t)

### Example 3-4

### 7-[2-(2-Methoxy-ethoxy)-6-propyl-thieno[2,3-d]pyrimidin-4-yl]-3-trifluoromethyl-5,6,7,8-tetrahydro-[1,2,4]triazolo[4,3-a]pyrazine

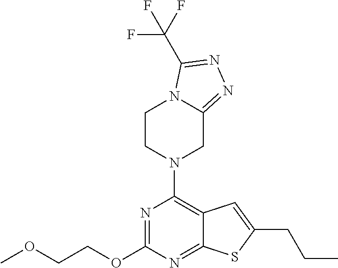

32 mg of the title compound (73%) was prepared according to the same method as Example 1-45 except that 2-methoxy-ethanol (39 mg, 0.51 mmol) was used instead of piperidine-3-carboxylic acid ethyl ester.

¹H NMR (500 MHz, CDCl₃); δ 6.93 (1H, s), 5.30 (2H, s), 4.53 (2H, t), 4.35 (2H, m), 4.32 (2H, m), 3.77 (2H, t), 3.43 (3H, s), 2.82 (2H, t), 1.75 (2H, m), 1.00 (3H, t)

### Example 3-5

### 7-[6-Propyl-2-(tetrahydro-furan-3-yloxy)-thieno[2,3-d]pyrimidin-4-yl]-3-trifluoromethyl-5,6,7,8-tetrahydro-[1,2,4]triazolo[4,3-a]pyrazine

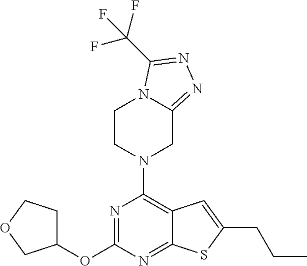

27 mg of the title compound (60%) was prepared according to the same method as Example 1-45 except that tetrahydrofuran-3-ol (18 mg, 0.2 mmol) was used instead of piperidine-3-carboxylic acid ethyl ester.

¹H NMR (400 MHz, CDCl₃); δ 6.94 (1H, s), 5.55 (1H, m), 5.29 (2H, s), 4.34 (4H, m), 4.10 (1H, dd), 4.07˜3.88 (3H, m), 2.84 (2H, t), 2.26 (2H, m), 1.75 (2H, m), 1.02 (3H, t)

### Example 3-6

### 7-[6-Propyl-2-(tetrahydro-furan-2-ylmethoxy)-thieno[2,3-d]pyrimidin-4-yl]-3-trifluoromethyl-5,6,7,8-tetrahydro-[1,2,4]triazolo[4,3-a]pyrazine

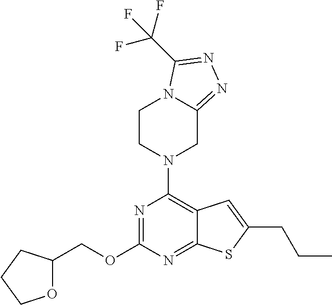

30 mg of the title compound (64%) was prepared according to the same method as Example 1-45 except that (tetrahydrofuran-2-yl)-methanol (20 mg, 0.2 mmol) was used instead of piperidine-3-carboxylic acid ethyl ester.

¹H NMR (400 MHz, CDCl₃); δ 6.93 (1H, s), 5.30 (2H, s), 4.40˜4.27 (7H, m), 3.94 (1H, dd), 3.82 (1H, dd), 2.83 (2H, t), 2.15˜1.70 (6H, m), 1.01 (3H, t)

### Example 3-7

### 7-[2-(1-Methyl-pyrrolidin-3-yloxy)-6-propyl-thieno[2,3-d]pyrimidin-4-yl]-3-trifluoromethyl-5,6,7,8-tetrahydro-[1,2,4]triazolo[4,3-a]pyrazine

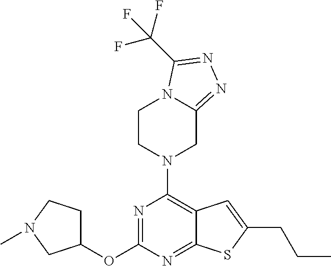

33 mg of the title compound (70%) was prepared according to the same method as Example 1-45 except that 1-methyl-pyrrolidin-3-ol (20 mg, 0.2 mmol) was used instead of piperidine-3-carboxylic acid ethyl ester.

¹H NMR (400 MHz, CDCl₃); δ 6.93 (1H, s), 5.44 (1H, m), 5.28 (2H, s), 4.33 (2H, m), 3.03 (1H, dd), 2.88˜2.72 (4H, m), 2.57 (1H, dd), 2.41 (3H, s), 2.37 (1H, m), 2.06 (1H, m), 1.75 (2H, m), 1.01 (3H, t)

### Example 3-8

### [6-Propyl-4-(3-trifluoromethyl-5,6-dihydro-8H-[1,2,4]triazolo[4,3-a]pyrazin-7-yl)-thieno[2,3-d]pyrimidin-2-yloxy]-acetic acid methyl ester

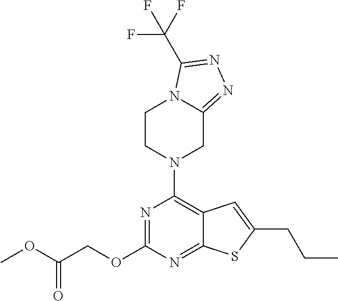

7 mg of the title compound (14%) was prepared according to the same method as Example 1-45 except that hydroxy-acetic acid methyl ester (19 mg, 0.21 mmol) was used instead of piperidine-3-carboxylic acid ethyl ester.

¹H NMR (500 MHz, CDCl₃); δ 6.94 (1H, s), 5.30 (2H, s), 4.92 (2H, s), 4.35 (2H, m), 4.30 (2H, m), 3.77 (3H, s), 2.82 (2H, t), 1.74 (2H, m), 1.00 (3H, t)

### Example 3-9

### [6-Propyl-4-(3-trifluoromethyl-5,6-dihydro-8H-[1,2,4]triazolo[4,3-a]pyrazin-7-yl)-thieno[2,3-d]pyrimidin-2-yloxy]-acetic acid

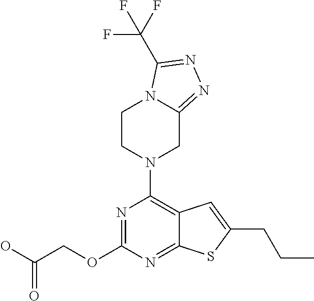

The compound (4.8 mg, 0.01 mmol) obtained from Example 3-8 was dissolved in tetrahydrofuran (1.5 mL) and methanol (0.5 mL), and then 1.0 N NaOH aqueous solution (0.1 mL, 0.1 mmol) was added thereto and stirred for 1 hour. The resulting solution was acidified with 6.0 N HCl aqueous solution, distilled under reduced pressure to remove solvent, and then diluted with ethyl acetate and washed with water and salt water. The organic layer was dried with anhydrous magnesium sulfate, distilled under reduced pressure and then purified by column chromatography using the 95:5 mixture of dichloromethane and methanol to give 3.6 mg of title compound (77%).

¹H NMR (500 MHz, CDCl₃); δ 6.80 (1H, s), 5.18 (2H, br s), 4.73 (2H, br s), 4.40-4.20 (4H, m), 2.71 (2H, t), 1.67 (2H, m), 1.00 (3H, t)

### Preparation Example 3-10-1

### 3-(tert-Butyl-dimethyl-silanyloxy)-propan-1-ol

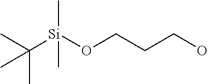

The title compound was prepared according to the known method (see J. Org. Chem., 51, 3388 (1986)).

### Preparation Example 3-10-2

### 7-{2-[3-(tert-Butyl-dimethyl-silanyloxy)-propoxy]-6-propyl-thieno[2,3-d]pyrimidin-4-yl}-3-trifluoromethyl-5,6,7,8-tetrahydro-[1,2,4]triazolo[4,3-a]pyrazine

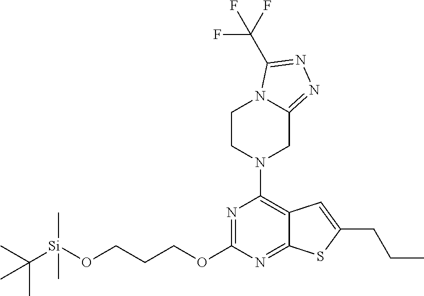

142 mg of the title compound (78%) was prepared according to the same method as Example 1-45 except that the compound (126 mg, 0.66 mmol) obtained from Preparation Example 3-10-1 was used instead of piperidine-3-carboxylic acid ethyl ester.

¹H NMR (500 MHz, CDCl₃); δ 6.93 (1H, s), 5.31 (2H, s), 4.46 (2H, t), 4.37 (2H, m), 4.33 (2H, m), 3.80 (2H, t), 2.83 (2H, t), 2.02 (2H, m), 1.74 (2H, m), 1.00 (3H, t), 0.88 (9H, s), 0.04 (6H, s)

### Example 3-10

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propan-1-ol

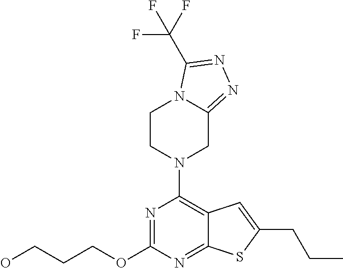

The compound (142 mg, 0.26 mmol) obtained from Preparation Example 3-10-2 was dissolved in tetrahydrofuran (7 mL), and then 1M tetrabutylammonium fluoride (0.4 mL, 0.4 mmol) was added thereto and stirred for 2 hours. Ethyl acetate (50 mL) was added thereto and washed with water (25 mL) twice. The organic layer was dried with anhydrous magnesium sulfate, distilled under reduced pressure and then purified by column chromatography using the 1:2 mixture of hexane and ethyl acetate to give 71 mg of title compound (60%).

¹H NMR (500 MHz, CDCl₃); δ 6.93 (1H, s), 5.30 (2H, s), 4.55 (2H, t), 4.36 (2H, m), 4.32 (2H, m), 3.78 (2H, t), 2.82 (2H, t), 2.04 (2H, m), 1.73 (2H, m), 1.00 (3H, t)

### Example 3-11

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid

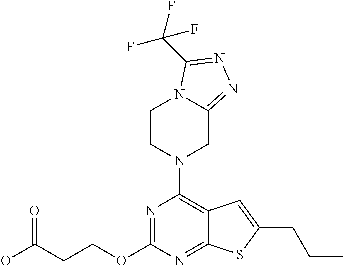

The compound (19 mg, 0.04 mmol) obtained from Example 3-10 was dissolved in acetonitrile (1.5 mL) and buffer (pH 6.7, 1 mL), and then 2,2,6,6-tetramethylpiperidine 1-oxy (2 mg), sodium chlorite (16 mg, 0.16 mmol) and sodium hypochlorite aqueous solution (0.05 mL) were added thereto and stirred for 72 hours. Ethyl acetate (15 mL) was added thereto and washed with water (5 mL) twice. The organic layer was dried with anhydrous magnesium sulfate, distilled under reduced pressure and then purified by column chromatography using ethyl acetate to give 13 mg of title compound (65%).

¹H NMR (500 MHz, CDCl₃); δ 6.94 (1H, s), 5.30 (2H, s), 4.64 (2H, t), 4.36 (2H, m), 4.30 (2H, m), 2.88 (2H, t), 2.82 (2H, t), 1.73 (2H, m), 0.99 (3H, t)

### Example 3-12

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid isopropyl ester

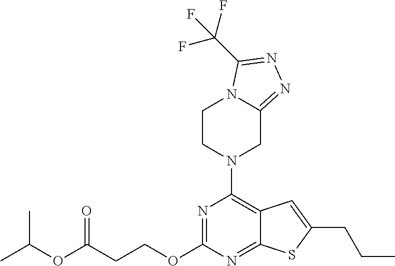

The compound (70 mg, 0.153 mmol) obtained from Example 3-11 was dissolved in isopropanol (10 mL), and then oxalyl chloride (0.5 L) was slowly added dropwise thereto and stirred for 4 hours with reflux. The resulting mixture was distilled under reduced pressure and then purified by column chromatography using the 1:4 mixture of ethyl acetate and dichloromethane to give 60 mg of title compound (78%).

¹H NMR (400 MHz, CDCl₃); δ 6.93 (1H, s), 5.30 (2H, s), 5.05 (1H, m), 4.64 (2H, t), 4.37 (2H, m), 4.30 (2H, m), 2.79˜2.85 (4H, m), 1.78 (2H, m), 1.26 (3H, s), 1.24 (3H, s), 1.01 (3H, t)

### Example 3-13

### 2,2-Dimethyl-propionic Acid

### 3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionyloxymethyl ester

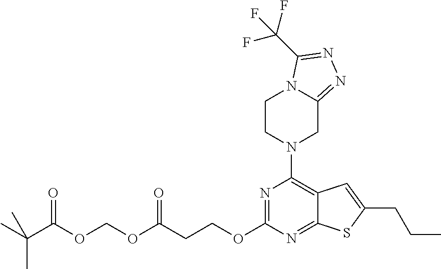

The compound (70 mg, 0.153 mmol) obtained from Example 3-11 and potassium carbonate (42 mg, 0.230 mmol) were diluted in N,N-dimethylformamide (5 mL), and then chloromethyl pivalate (33 μl, 0.230 mmol) was added thereto and stirred at 70° C. for 4 hours. Ethyl acetate (50 mL) was added thereto and washed with water (50 mL) twice. The resulting mixture was distilled under reduced pressure and then purified by column chromatography using the 1:4 mixture of dichloromethane and ethyl acetate to give 55 mg of title compound (63%).

¹H NMR (400 MHz, CDCl₃); δ 6.94 (1H, s), 5.79 (2H, s), 5.30 (2H, s), 4.66 (2H, t), 4.36 (2H, m), 4.31 (2H, m), 2.90 (2H, t), 2.83 (2H, t), 1.76 (2H, m), 1.20 (9H, s), 1.01 (3H, t)

### Preparation Example 3-14-1

### 3,3-Dimethoxy-propan-1-ol

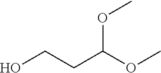

3,3-dimethoxy-propionic acid methyl ester (5 g, 33.7 mmol) was diluted in diethyl ether (100 mL) and then cooled to 0° C. Lithium aluminum hydride (1.41 g, 37.2 mmol) was slowly added thereto and stirred for 1 hour. Water (1.4 mL), 4 N NaOH aqueous solution (1.4 mL) and water (4.2 mL) were added thereto sequentially and then stirred for 1 hour. The produced solid was filtered and then washed with diethylether several times. The collected filtrate was distilled under reduced pressure to give 4.05 g of title compound (99%).

¹H NMR (500 MHz, CDCl₃); δ 4.54 (1H, t), 3.73 (2H, t), 3.36 (6H, s), 1.87 (2H, m)

### Example 3-14

### 7-[2-(3,3-Dimethoxy-propoxy)-6-propyl-thieno[2,3-d]pyrimidin-4-yl]-3-trifluoromethyl-5,6,7,8-tetrahydro-[1,2,4]triazolo[4,3-a]pyrazine

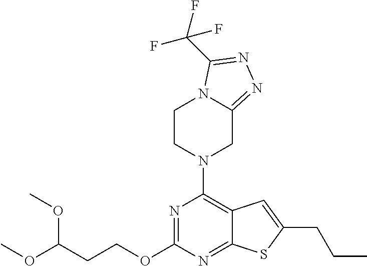

76 mg of the title compound (70%) was prepared according to the same method as Example 1-45 except that the compound (54 mg, 0.45 mmol) obtained from Preparation Example 3-14-1 was used instead of piperidine-3-carboxylic acid ethyl ester.

¹H NMR (500 MHz, CDCl₃); δ 6.92 (1H, s), 5.29 (2H, s), 4.65 (1H, t), 4.43 (2H, t), 4.37 (2H, m), 4.30 (2H, m), 3.36 (6H, s), 2.82 (2H, t), 2.13 (2H, m), 1.74 (2H, m), 1.00 (3H, t)

### Example 3-15

### 7-(2-Cyclopentyloxy-6-propyl-thieno[2,3-d]pyrimidin-4-yl)-3-trifluoromethyl-5,6,7,8-tetrahydro-[1,2,4]triazolo[4,3-a]pyrazine

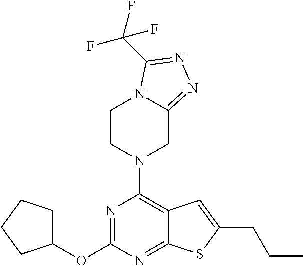

31 mg of the title compound (69%) was prepared according to the same method as Example 1-45 except that cyclopentanol (17 mg, 0.2 mmol) was used instead of piperidine-3-carboxylic acid ethyl ester.

¹H NMR (400 MHz, CDCl₃); δ 6.90 (1H, s), 5.39 (1H, m), 5.27 (2H, s), 4.36 (2H, t), 4.29 (2H, t), 2.81 (2H, t), 2.40˜1.58 (10H, m), 1.00 (3H, t)

### Example 3-16

### 7-(2-Benzyloxy-6-propyl-thieno[2,3-d]pyrimidin-4-yl)-3-trifluoromethyl-5,6,7,8-tetrahydro-[1,2,4]triazolo[4,3-a]pyrazine

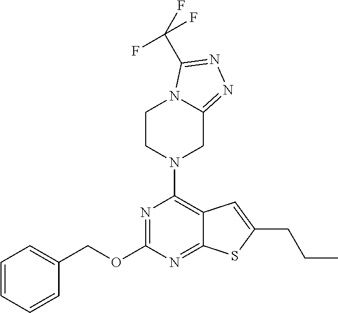

11 mg of the title compound (23%) was prepared according to the same method as Example 1-45 except that benzyl alcohol (22 mg, 0.2 mmol) was used instead of piperidine-3-carboxylic acid ethyl ester.

¹H NMR (500 MHz, CDCl₃); δ 7.46 (2H, d), 7.35 (2H, t), 7.29 (1H, t), 6.92 (1H, s), 5.43 (2H, s), 5.27 (2H, s), 4.25 (4H, s), 2.82 (2H, t), 1.74 (2H, m), 1.00 (3H, t)

### Example 3-17

### 7-(2-Butoxy-6-propyl-thieno[2,3-d]pyrimidin-4-yl)-3-trifluoromethyl-5,6,7,8-tetrahydro-[1,2,4]triazolo[4,3-a]pyrazine

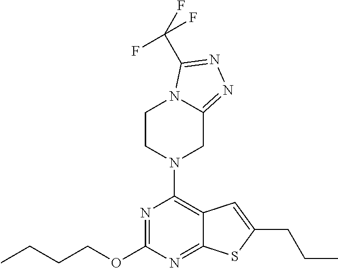

Butyl alcohol (2 mL) was added to the compound (49 mg, 0.12 mmol) obtained from Preparation Example 1-1-3 and sodium cyanide (11.9 mg, 0.24 mmol), heated to 150° C. in a microwave reactor, and the reaction was carried out for 1 hour. The resulting mixture was distilled under reduced pressure and then purified by column chromatography using the 2:1 mixture of hexane and ethyl acetate to give 6 mg of title compound (11%).

¹H NMR (500 MHz, CDCl₃); δ 6.92 (1H, s), 5.28 (2H, s), 4.36 (4H, m), 4.29 (2H, m), 2.82 (2H, t), 1.81 (2H, m), 1.75 (2H, m), 1.49 (2H, m), 1.00 (3H, t), 0.97 (3H, t)

### Preparation Example 3-18-1

### [2-((E)-Styryl)-oxazol-4-yl]-acetic acid ethyl ester

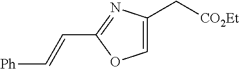

(E)-3-phenylacrylamide (25.5 g, 173 mmol) was added to ethyl 4-chloroacetoacetate (51.3 g, 311 mmol) and stirred at 100° C. for 16 hours. Ethyl acetate (300 mL) was added thereto, and the resulting mixture was extracted with ethyl acetate and water, and then distilled under reduced pressure to give 29.8 mg of title compound (67%).

¹H NMR (400 MHz, CDCl₃); δ 7.62 (s, 1H), 7.52 (m, 2H), 7.49 (d, 1H), 7.40˜7.33 (m, 3H), 6.92 (d, 1H), 4.37 (q, 2H), 3.64 (d, 2H), 1.29 (t, 3H)

### Preparation Example 3-18-2

### (2-Hydroxymethyl-oxazol-4-yl)-acetic acid ethyl ester

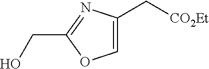

The compound (10.2 g, 39.6 mmol) obtained from Preparation Example 3-18-1 was dissolved in the 2:1 mixture (300 mL) of tetrahydrofuran and water, and then catalytic amount of osmium tetroxide (102 mg, 0.40 mmol) and sodium periodate (25.4 g, 119 mmol) were added thereto. The resulting mixture was stirred at room temperature for 16 hours and filtrated, and then washed with water and distilled under reduced pressure to obtain unpurified aldehyde compound. Ethanol (50 mL) was added to the obtained compound and then sodium borohydride (2.95 g, 79.3 mmol) was added thereto. The resulting mixture was stirred at room temperature for 5 hours, distilled under reduced pressure to remove ethanol and then washed with dichloromethane and water. The organic layer was dried with anhydrous magnesium sulfate, distilled under reduced pressure and then purified by column chromatography using the 95:5 mixture of dichloromethane and methanol to give 4.50 g of title compound (61%).

¹H NMR (400 MHz, CDCl₃); δ 7.62 (s, 1H), 4.71 (d, 2H), 4.20 (q, 2H), 3.60 (d, 2H), 3.07 (br s, 1H), 1.28 (t, 3H)

### Example 3-18

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-oxazol-4-yl}-acetic acid ethyl ester

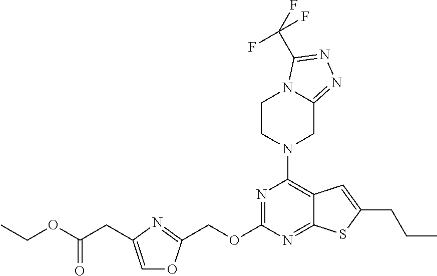

24 mg of the title compound (35%) was prepared according to the same method as Example 1-45 except that the compound (46 mg, 0.25 mmol) obtained from Preparation Example 3-18-2 was used instead of piperidine-3-carboxylic acid ethyl ester.

¹H NMR (500 MHz, CDCl₃); δ 7.63 (1H, s), 6.95 (1H, s), 5.46 (2H, s), 5.30 (2H, s), 4.34 (2H, m), 4.30 (2H, m), 4.17 (2H, q), 3.60 (2H, s), 2.82 (2H, t), 1.73 (2H, m), 1.27 (3H, t), 1.00 (3H, t)

### Example 3-19

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-oxazol-4-yl}-acetic acid

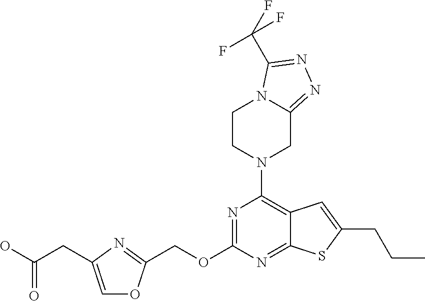

The compound (21 mg, 0.04 mmol) obtained from Example 3-18 was dissolved in tetrahydrofuran (3 mL) and methanol (1 mL), and then 1.0 N NaOH aqueous solution (0.1 mL, 0.1 mmol) was added thereto and stirred for 1 hour. The resulting solution was acidified with 6.0 N HCl aqueous solution, distilled under reduced pressure to remove solvent, and then diluted with ethyl acetate and washed with water and salt water. The organic layer was dried with anhydrous magnesium sulfate, distilled under reduced pressure and then purified by column chromatography using the 95:5 mixture of dichloromethane and methanol to give 9 mg of title compound (45%).

¹H NMR (500 MHz, CDCl₃); δ 7.78 (1H, s), 7.22 (1H, s), 5.45 (2H, s), 5.26 (2H, s), 4.39 (2H, m), 4.31 (2H, m), 3.55 (2H, s), 2.84 (2H, t), 1.73 (2H, m), 1.00 (3H, t)

### Example 3-20

### 7-[2-(Oxazol-4-ylmethoxy)-6-propyl-thieno[2,3-d]pyrimidin-4-yl]-3-trifluoromethyl-5,6,7,8-tetrahydro-[1,2,4]triazolo[4,3-a]pyrazine

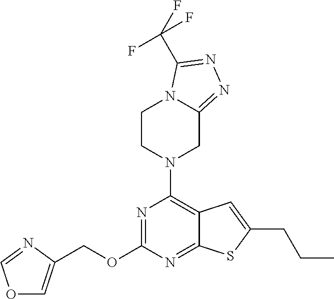

30 mg of the title compound (64%) was prepared according to the same method as Example 1-45 except that oxazole-4-yl-methanol (20 mg, 0.2 mmol) was used instead of piperidine-3-carboxylic acid ethyl ester.

¹H NMR (500 MHz, CDCl₃); δ 7.86 (1H, s), 7.76 (1H, s), 6.94 (1H, s), 5.38 (2H, s), 5.29 (2H, s), 4.32 (4H, m), 2.82 (2H, t), 1.74 (2H, m), 1.00 (3H, t)

### Example 3-21

### 7-{6-Propyl-2-[2-(2,3,5-trifluoro-phenyl)-ethoxy]-thieno[2,3-d]pyrimidin-4-yl}-3-trifluoromethyl-5,6,7,8-tetrahydro-[1,2,4]triazolo[4,3-a]pyrazine

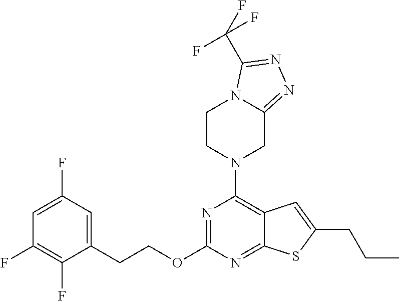

35 mg of the title compound (65%) was prepared according to the same method as Example 1-45 except that 2-(2,3,5-trifluoro-phenyl)-ethanol (35 mg, 0.2 mmol) was used instead of piperidine-3-carboxylic acid ethyl ester.

¹H NMR (500 MHz, CDCl₃); δ 7.13 (1H, d), 6.93 (1H, s), 6.89 (1H, m), 5.29 (2H, s), 4.54 (2H, t), 4.36 (2H, t), 4.30 (2H, t), 3.10 (2H, t), 2.82 (2H, t), 1.74 (2H, m), 1.00 (3H, t)

### Example 3-22

### 7-[2-(Indan-2-yloxy)-6-propyl-thieno[2,3-d]pyrimidin-4-yl]-3-trifluoromethyl-5,6,7,8-tetrahydro-[1,2,4]triazolo[4,3-a]pyrazine

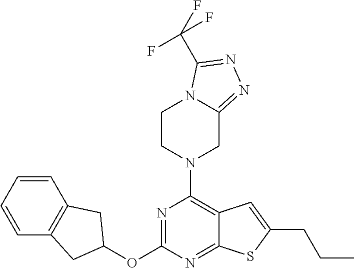

21 mg of the title compound (42%) was prepared according to the same method as Example 1-45 except that indan-2-ol (27 mg, 0.2 mmol) was used instead of piperidine-3-carboxylic acid ethyl ester.

¹H NMR (500 MHz, CDCl₃); δ 7.24˜7.15 (4H, m), 6.93 (1H, s), 5.79 (1H, m), 5.28 (2H, s), 4.34 (2H, t), 4.29 (2H, t), 3.43 (2H, dd), 3.20 (2H, dd), 2.83 (2H, t), 1.75 (2H, m), 1.01 (3H, t)

### Example 3-23

### 7-[6-Propyl-2-(pyridin-2-ylmethoxy)-thieno[2,3-d]pyrimidin-4-yl]-3-trifluoromethyl-5,6,7,8-tetrahydro-[1,2,4]triazolo[4,3-a]pyrazine

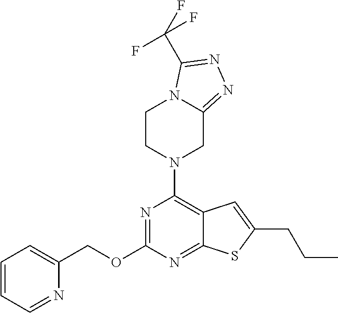

21 mg of the title compound (44%) was prepared according to the same method as Example 1-45 except that pyridin-2-yl-methanol (22 mg, 0.2 mmol) was used instead of piperidine-3-carboxylic acid ethyl ester.

¹H NMR (500 MHz, CDCl₃); δ 7.66 (1H, dd), 7.48 (1H, d), 7.20 (1H, dd), 6.93 (1H, s), 5.54 (2H, m), 5.28 (2H, s), 4.25 (2H, t), 4.18 (2H, t), 2.81 (2H, t), 1.74 (2H, m), 0.99 (3H, t)

### Example 3-24

### 7-[6-Propyl-2-(pyridin-3-ylmethoxy)-thieno[2,3-d]pyrimidin-4-yl]-3-trifluoromethyl-5,6,7,8-tetrahydro-[1,2,4]triazolo[4,3-a]pyrazine

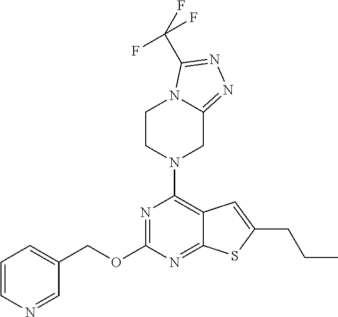

41 mg of the title compound (85%) was prepared according to the same method as Example 1-45 except that pyridin-3-yl-methanol (22 mg, 0.2 mmol) was used instead of piperidine-3-carboxylic acid ethyl ester.

¹H NMR (500 MHz, CDCl₃); δ 8.72 (1H, s), 8.54 (1H, d), 7.82 (1H, d), 7.29 (1H, dd), 6.94 (1H, s), 5.44 (1H, s), 5.28 (2H, s), 4.28 (4H, m), 2.82 (2H, t), 1.74 (2H, m), 1.00 (3H, t)

### Example 3-25

### 7-[6-Propyl-2-(pyridin-4-ylmethoxy)-thieno[2,3-d]pyrimidin-4-yl]-3-trifluoromethyl-5,6,7,8-tetrahydro-[1,2,4]triazolo[4,3-a]pyrazine

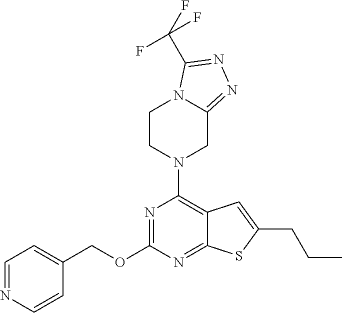

40 mg of the title compound (83%) was prepared according to the same method as Example 1-45 except that pyridin-4-yl-methanol (22 mg, 0.2 mmol) was used instead of piperidine-3-carboxylic acid ethyl ester.

¹H NMR (500 MHz, CDCl₃); δ 8.57 (2H, d), 7.36 (2H, d), 6.93 (1H, s), 5.43 (2H, s), 5.27 (2H, s), 4.29 (4H, m), 2.82 (2H, t), 1.73 (2H, m), 0.99 (3H, t)

### Preparation Example 3-26-1

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-azetidine-1-carboxylic acid tert-butyl ester

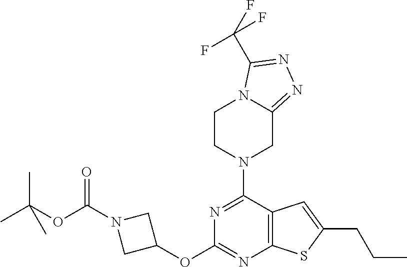

38 mg of the title compound (70%) was prepared according to the same method as Example 1-45 except that the compound (35 mg, 0.2 mmol) obtained from Preparation Example 1-83-2 was used instead of piperidine-3-carboxylic acid ethyl ester.

¹H NMR (400 MHz, CDCl₃); δ 6.96 (1H, s), 5.33 (1H, m), 5.30 (2H, s), 4.42˜4.28 (6H, m), 4.05 (2H, dd), 2.84 (2H, t), 1.75 (2H, m), 1.01 (3H, t)

### Example 3-26

### 7-[2-(Azetidin-3-yloxy)-6-propyl-thieno[2,3-d]pyrimidin-4-yl]-3-trifluoromethyl-5,6,7,8-tetrahydro-[1,2,4]triazolo[4,3-a]pyrazine

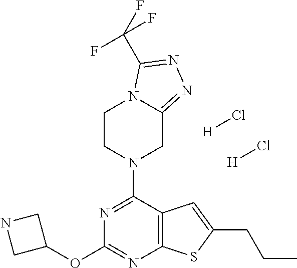

30 mg of the title compound (83%) was prepared according to the same method as Example 1-4 except that the compound (38 mg, 0.07 mmol) obtained from Preparation Example 3-26-1 was used instead of the compound obtained from Preparation Example 1-4-1.

¹H NMR (400 MHz, DMSO, d₆); δ 9.27 (1H, br s), 9.08 (1H, br s), 7.45 (1H, s), 5.39 (1H, m), 5.22 (2H, s), 4.46˜4.26 (6H, m), 4.05 (2H, m), 2.85 (2H, t), 1.68 (2H, m), 0.95 (3H, t)

### Example 3-27

### 6-Propyl-4-(3-trifluoromethyl-5,6-dihydro-8H-[1,2,4]triazolo[4,3-a]pyrazin-7-yl)-thieno[2,3-d]pyrimidin-2-ol

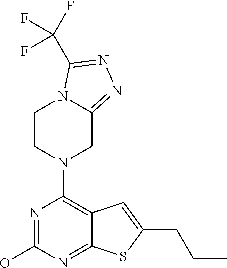

The compound (66 mg, 0.14 mmol) obtained from Example 3-14 was dissolved in dichloromethane (5 mL), and then p-toluenesulfonic acid hydrate (28 mg, 0.15 mmol) was added thereto and stirred for 1 hour. The resulting mixture was diluted with ethyl acetate (15 mL) and washed with water and salt water. The organic layer was dried with anhydrous magnesium sulfate, distilled under reduced pressure and then purified by column chromatography using the 1:2 mixture of hexane and ethyl acetate to give 3.1 mg of title compound (6%).

¹H NMR (500 MHz, CDCl₃); δ 6.88 (1H, s), 5.34 (2H, s), 4.39 (4H, m), 2.76 (2H, t), 1.70 (2H, m), 0.99 (3H, t)

### Example 3-28

### 7-(2-Phenoxy-6-propyl-thieno[2,3-d]pyrimidin-4-yl)-3-trifluoromethyl-5,6,7,8-tetrahydro-[1,2,4]triazolo[4,3-a]pyrazine

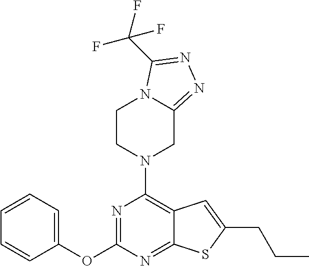

24 mg of the title compound (52%) was prepared according to the same method as Example 1-45 except that phenol (19 mg, 0.2 mmol) was used instead of piperidine-3-carboxylic acid ethyl ester.

### Example 3-29

### 7-[6-Propyl-2-(pyridin-3-yloxy)-thieno[2,3-d]pyrimidin-4-yl]-3-trifluoromethyl-5,6,7,8-tetrahydro-[1,2,4]triazolo[4,3-a]pyrazine

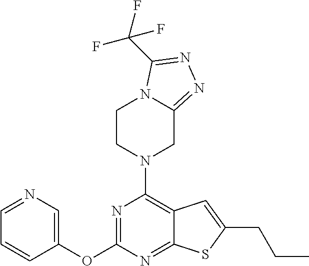

10 mg of the title compound (17%) was prepared according to the similar method with Example 1-45 by using the compound (50 mg, 0.124 mmol) obtained from Preparation Example 1-1-3 and 3-hydroxypyridin (11.8 mg, 0.124 mmol).

¹H NMR (400 MHz, CDCl₃); δ 8.56 (1H, m), 8.50 (1H, m), 7.55 (1H, m), 7.37 (1H, m), 6.99 (1H, s), 4.26 (2H, m), 4.21 (2H, m), 2.84 (2H, t), 1.75 (2H, m), 1.01 (3H, t)

### Example 3-30

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester

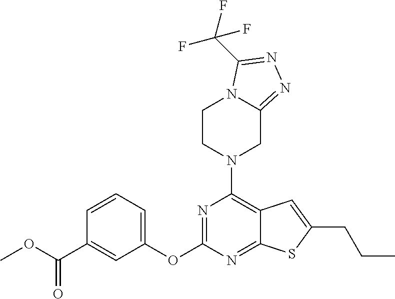

47 mg of the title compound (73%) was prepared according to the similar method with Example 1-45 by using the compound (50 mg, 0.124 mmol) obtained from Preparation Example 1-1-3 and 3-hydroxy-benzoic acid methyl ester (19.0 mg, 0.124 mmol).

¹H NMR (400 MHz, CDCl₃); δ 7.92 (1H, m), 7.89 (1H, m), 7.49 (1H, m), 7.38 (1H, m), 6.97 (1H, s), 5.28 (2H, s), 4.21 (2H, m), 4.17 (2H, m), 2.84 (2H, t), 1.75 (2H, m), 1.01 (3H, t)

### Example 3-31

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid

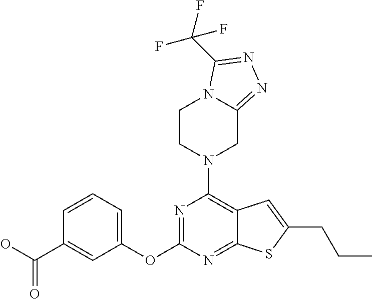

The compound (40 mg, 0.077 mmol) obtained from Example 3-30 was dissolved in the 5:3:1 mixture of tetrahydrofuran, water and methanol, and lithum hydroxide (6.5 mg, 0.154 mmol) was added thereto and stirred at room temperature for 4 hours. The resulting mixture was distilled under reduced pressure and then purified by column chromatography using the 1:1 mixture of ethyl acetate and dichloromethane to give 9 mg of title compound (23%).

¹H NMR (400 MHz, CDCl₃); δ 7.96 (2H, br), 7.48 (1H, br), 7.41 (1H, br), 6.98 (1H, s), 5.28 (2H, s), 4.24 (2H, m), 4.18 (2H, m), 2.83 (2H, t), 1.75 (2H, m), 1.01 (3H, t)

### Example 3-32

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester

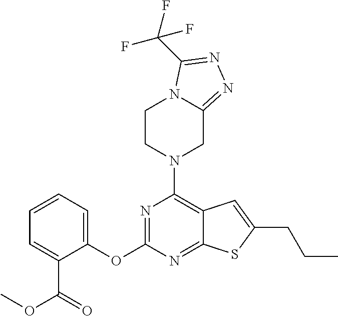

10 mg of the title compound (16%) was prepared according to the similar method with Example 1-45 by using the compound (50 mg, 0.124 mmol) obtained from Preparation Example 1-1-3 and 2-hydroxy-benzoic acid methyl ester (19.0 mg, 0.124 mmol).

¹H NMR (400 MHz, CDCl₃); δ 7.98 (1H, m), 7.59 (1H, m), 7.33 (1H, m), 7.22 (1H, m), 6.94 (1H, s), 5.23 (2H, s), 4.18 (2H, m), 4.12 (2H, m), 3.65 (3H, s), 2.824 (2H, t), 1.75 (2H, m), 1.01 (3H, t)

### Example 3-33

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid

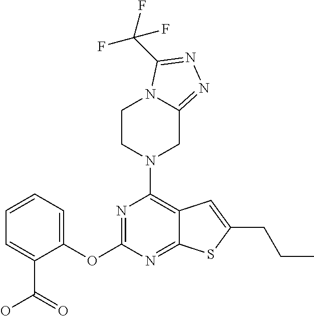

0.8 mg of the title compound (8%) was prepared according to the similar method with Example 3-31 by using the compound (10 mg, 0.019 mmol) obtained from Example 3-32.

¹H NMR (400 MHz, CDCl₃); δ 8.08 (1H, m), 7.62 (1H, m), 7.38 (1H, m), 7.26 (1H, m), 6.96 (1H, s), 5.24 (2H, s), 4.18 (2H, m), 4.16 (2H, m), 2.84 (2H, t), 1.75 (2H, m), 1.01 (3H, t)

### Example 3-34

### 4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid methyl ester

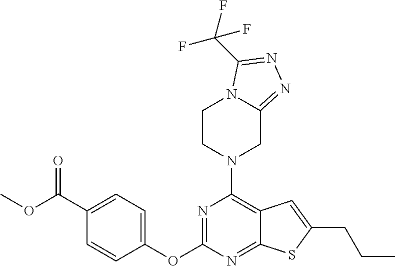

30 mg of the title compound (47%) was prepared according to the similar method with Example 1-45 by using the compound (50 mg, 0.124 mmol) obtained from Preparation Example 1-1-3 and 4-hydroxy-benzoic acid methyl ester (19.0 mg, 0.124 mmol).

¹H NMR (500 MHz, CDCl₃); δ 8.08 (2H, d), 7.23 (2H, d), 6.98 (1H, s), 5.28 (2H, s), 4.24 (2H, m), 4.19 (2H, m), 3.93 (3H, s), 2.84 (2H, t), 1.75 (2H, m), 1.01 (3H, t)

### Example 3-35

### 4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzoic acid

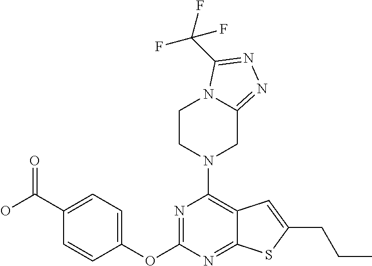

10 mg of the title compound (34%) was prepared according to the similar method with Example 3-31 by using the compound (30 mg, 0.058 mmol) obtained from Example 3-34.

¹H NMR (500 MHz, CDCl₃); δ 8.15 (2H, d), 7.07 (2H, d), 7.00 (1H, s), 5.31 (2H, s), 4.27 (2H, m), 4.24 (2H, m), 2.85 (2H, t), 1.75 (2H, m), 1.01 (3H, t)

### Example 3-36

### 7-[6-Propyl-2-(pyridin-2-yloxy)-thieno[2,3-d]pyrimidin-4-yl]-3-trifluoromethyl-5,6,7,8-tetrahydro-[1,2,4]triazolo[4,3-a]pyrazine

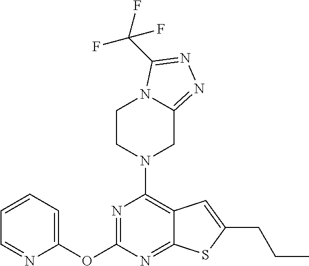

34 mg of the title compound (59%) was prepared according to the similar method with Example 1-45 by using the compound (50 mg, 0.124 mmol) obtained from Preparation Example 1-1-3 and 2-hydroxypyridin (11.8 mg, 0.124 mmol).

¹H NMR (400 MHz, CDCl₃); δ 8.32 (1H, m), 7.81 (1H, m), 7.18 (1H, m), 7.10 (1H, d), 6.98 (1H, m), 5.27 (2H, s), 4.26 (2H, m), 4.20 (2H, m), 2.85 (2H, t), 1.75 (2H, m), 1.01 (3H, t)

### Example 3-37

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzonitrile

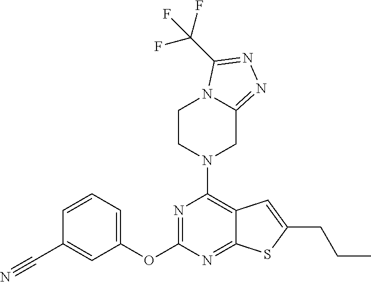

5 mg of the title compound (8%) was prepared according to the similar method with Example 1-45 by using the compound (50 mg, 0.124 mmol) obtained from Preparation Example 1-1-3 and 3-hydroxybenzonitrile (29.57 mg, 0.248 mmol).

¹H NMR (500 MHz, CDCl₃); δ 7.71 (1H, m), 7.64 (1H, m), 7.35 (1H, m), 7.29 (1H, m), 6.98 (1H, m), 5.25 (2H, s), 4.25 (4H, m), 2.84 (2H, t), 1.75 (2H, m), 1.01 (3H, t)

### Example 3-38

### 4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzonitrile

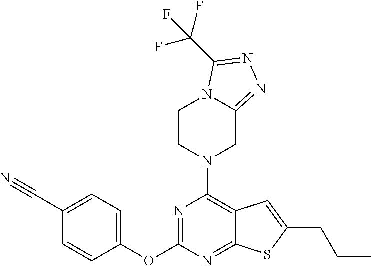

10 mg of the title compound (17%) was prepared according to the similar method with Example 1-45 by using the compound (50 mg, 0.124 mmol) obtained from Preparation Example 1-1-3 and 4-hydroxybenzonitrile (29.57 mg, 0.248 mmol).

¹H NMR (400 MHz, CDCl₃); δ 7.71 (2H, d), 7.30 (2H, d), 7.01 (1H, s), 5.31 (2H, s), 4.30 (2H, m), 4.24 (2H, m), 2.86 (2H, t), 1.77 (2H, m), 1.01 (3H, t)

### Example 3-39

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-acetic acid methyl ester

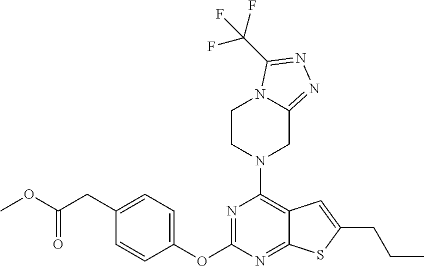

15 mg of the title compound (23%) was prepared according to the similar method with Example 1-45 by using the compound (50 mg, 0.124 mmol) obtained from Preparation Example 1-1-3 and (4-hydroxyphenyl)-acetic acid methyl ester (20.6 mg, 0.248 mmol).

¹H NMR (400 MHz, CDCl₃); δ 7.30 (2H, d), 7.13 (2H, d), 6.96 (1H, s), 5.26 (2H, s), 4.19 (2H, m), 4.14 (2H, m), 3.73 (3H, s), 3.67 (2H, s), 2.84 (2H, t), 1.74 (2H, m), 1.01 (3H, t)

### Example 3-40

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-acetic acid

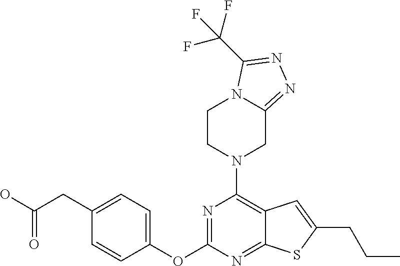

5 mg of the title compound (43%) was prepared according to the similar method with Example 3-31 by using the compound (12 mg, 0.023 mmol) obtained from Example 3-39.

¹H NMR (400 MHz, CDCl₃); δ 7.32 (2H, d), 7.10 (2H, d), 6.94 (1H, s), 5.16 (2H, s), 4.18 (2H, m), 4.12 (2H, m), 3.69 (2H, s), 2.83 (2H, t), 1.75 (2H, m), 1.00 (3H, t)

### Example 3-41

### 3-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-propionic acid methyl ester

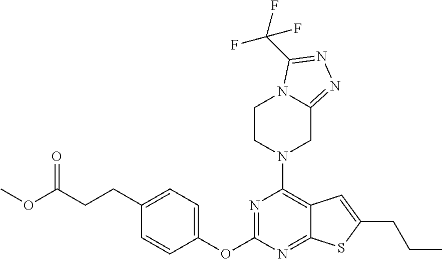

42 mg of the title compound (62%) was prepared according to the similar method with Example 1-45 by using the compound (50 mg, 0.124 mmol) obtained from Preparation Example 1-1-3 and 3-(4-hydroxyphenyl)-propionic acid methyl ester (44.7 mg, 0.248 mmol).

¹H NMR (500 MHz, CDCl₃); δ 7.21 (2H, d), 7.08 (2H, d), 6.94 (1H, s), 5.26 (2H, s), 4.22 (2H, m), 4.15 (2H, m), 3.68 (3H, s), 2.98 (2H, t), 2.82 (2H, t), 2.66 (2H, t), 1.76 (2H, m), 0.99 (3H, t)

### Example 3-42

### 3-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-propionic acid

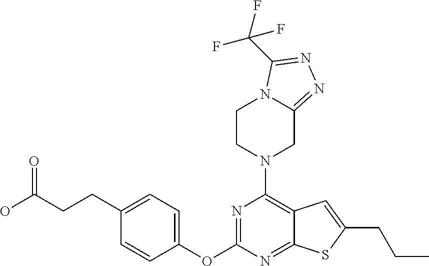

The compound obtained from Example 3-41 (35 mg, 0.064 mmol) was reacted according to the similar method to Example 3-31 to give the title compound (25 mg, 73%).

¹H NMR (400 MHz, CDCl₃); δ 7.30 (2H, d), 7.03 (2H, d), 6.86 (1H, s), 4.74 (2H, s), 4.31 (2H, m), 4.17 (2H, m), 3.03 (2H, t), 2.84 (2H, t), 2.76 (2H, t), 1.75 (2H, m), 1.01 (3H, t)

### Example 3-43

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenoxy}-acetic acid methyl ester

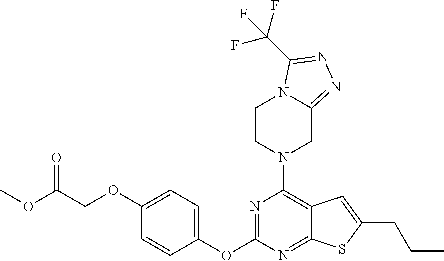

The compound obtained from Preparation Example 1-1-3 (50 mg, 0.124 mmol) and (4-hydroxyphenyl)-acetic acid methyl ester (45.2 mg, 0.248 mmol) were reacted according to the similar method to Example 1-45 to give the title compound (41 mg, 60%).

¹H NMR (500 MHz, CDCl₃); δ 7.09 (2H, d), 6.92 (3H, m), 5.26 (2H, s), 4.66 (2H, s), 4.22 (2H, m), 4.14 (2H, m), 3.82 (3H, s), 2.82 (2H, t), 1.73 (2H, m), 0.99 (3H, t)

### Example 3-44

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenoxy}-acetic acid

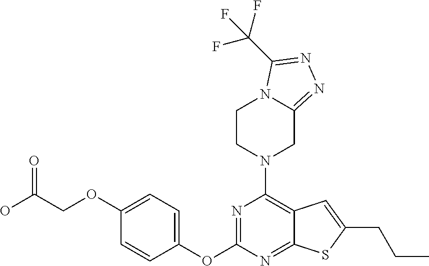

The compound obtained from Example 3-43 (35 mg, 0.064 mmol) was reacted according to the similar method to Example 3-31 to give the title compound (7 mg, 21%).

¹H NMR (400 MHz, CDCl₃); δ 6.96 (4H, br), 6.86 (1H, br), 4.90 (2H, br), 4.61 (2H, br), 4.26 (2H, br), 4.13 (2H, br), 2.79 (2H, br), 1.70 (2H, br), 0.98 (3H, t)

### Preparation Example 3-45-1

### 4-(3-Hydroxy-phenyl)-piperazine-1-carboxylic acid tert-butyl ester

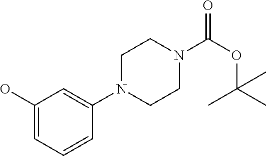

3-piperazine-1-yl-phenol (1.0 g, 5.61 mmol) and di-t-butyl dicarbonate (1.1 g, 5.05 mmol) were dissolved in dichloromethane (30 mL) and stirred for 4 hours at room temperature. The resulting mixture was washed with 1N aqueous hydrochloric acid and saturated aqueous sodium hydrogen carbonate solution, then dried with anhydrous sodium sulfate and distilled under reduced pressure to give the title compound (1.0 mg, 64%).

¹H NMR (400 MHz, CDCl₃); δ 7.11 (1H, t), 6.49 (1H, m), 6.40 (1H, m), 6.35 (1H, m), 5.25 (4H, m), 3.57 (4H, m), 3.11 (4H, m), 1.49 (9H, s)

### Example 3-45

### 7-[2-(3-piperazin-1-yl-phenoxy)-6-propyl-thieno[2,3-d]pyrimidin-4-yl]-3-trifluoromethyl-5,6,7,8-tetrahydro-[1,2,4]triazolo[4,3-a]pyrazine

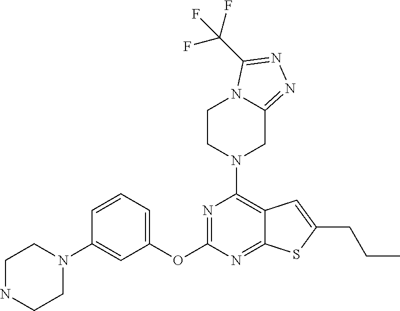

The compound obtained from Preparation Example 1-1-3 (50 mg, 0.124 mmol) and the compound obtained from Preparation Example 3-45-1 (69 mg, 0.248 mmol), palladium acetated (II) (2.79 mg, 0.012 mmol), BINAP (11.59 mg, 0.0186 mmol) and cesium carbonate (61 mg, 0.186 mmol) were diluted with toluene (5 mL) and then stirred under reflux for 3 hours. The resulting solution was cooled to room temperature, filtrated through Celite, distilled under reduced pressure to remove solvent, and then purified by column chromatography with the 1:1 mixture of hexane and ethyl acetate to give 4-{3-[6-propyl-4-(3-trifluoromethyl-5,6-dihydro-8H-[1,2,4]triazolo[4,3-a]pyrazine-7-yl)-thieno[2,3-d]pyrimidin-2-yloxy]-phenyl}-piperazine-1-carboxylic acid t-butyl ester (54 mg).

Thus obtained 4-{3-[6-propyl-4-(3-trifluoromethyl-5,6-dihydro-8H-[1,2,4]triazolo[4,3-a]pyrazine-7-yl)-thieno[2,3-d]pyrimidin-2-yloxy]-phenyl}-piperazine-1-carboxylic acid t-butyl ester (54 mg, 0.084 mmol) was dissolved in 4.0 M hydrochloric dioxan solution (5 mL) and stirred for 1 hours. The resulting mixture was distilled under reduced pressure and dried under vacuum to give the title compound (55 mg, step 2: 72%).

¹H NMR (500 MHz, DMSO, d₆); δ 8.95 (2H, br), 7.43 (1H, s), 7.25 (1H, t), 6.81 (1H, m), 6.79 (1H, m), 6.63 (1H, m), 5.12 (2H, s), 4.28 (2H, m), 4.21 (2H, m), 3.34 (4H, m), 3.16 (4H, m), 2.81 (2H, t), 1.63 (2H, m), 0.91 (3H, t)

### Preparation Example 3-46-1

### 4-(4-Hydroxy-phenyl)-piperazine-1-carboxylic acid tert-butyl ester

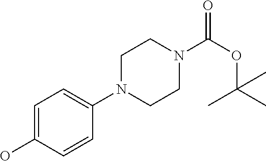

4-piperazine-1-yl-phenol (1.0 g, 5.61 mmol) and di-t-butyl dicarbonate (1.1 g, 5.05 mmol) were dissolved in dichloromethane (30 mL) and stirred for 4 hours at room temperature. The resulting mixture was washed with 1N aqueous hydrochloric acid and aqueous saturated sodium hydrogen carbonate solution, then dried with anhydrous sodium sulfate and distilled under reduced pressure to give the title compound (500 mg, 32%).

¹H NMR (400 MHz, CDCl₃); δ 6.84 (2H, d), 6.76 (1H, d), 4.87 (1H, s), 3.57 (4H, m), 2.99 (4H, m), 1.48 (9H, s)

### Example 3-46

### 7-[2-(4-piperazin-1-yl-phenoxy)-6-propyl-thieno[2,3-d]pyrimidin-4-yl]-3-trifluoromethyl-5,6,7,8-tetrahydro-[1,2,4]triazolo[4,3-a]pyrazine

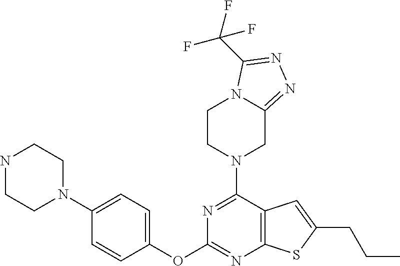

The compound obtained from Preparation Example 1-1-3 (50 mg, 0.124 mmol) and the compound obtained from Preparation Example 3-46-1 (69 mg, 0.248 mmol) were reacted according to the similar method to Example 3-45 to give the title compound (10 mg, step 2: 13%).

¹H NMR (500 MHz, DMSO, d₆); δ 8.93 (2H, br), 7.41 (1H, s), 7.05 (2H, d), 6.99 (2H, d), 5.11 (2H, s), 4.28 (2H, m), 4.21 (2H, m), 3.31 (4H, m), 3.22 (4H, m), 2.80 (2H, t), 1.64 (2H, m), 0.92 (3H, t)

### Example 3-47

### 4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-benzaldehyde

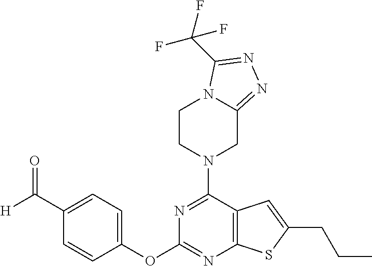

The compound obtained from Example 3-35 (30 mg, 0.059 mmol) was dissolved in tetrahydrofuran (5 mL) and lithium aluminum hydride (2.71 mg, 0.071 mmol) was added thereto in portion at 0° C. and then stirred for 1 hour at room temperature. Water (2.71 mL), 15% aqueous sodium hydroxide solution (2.71 mL) and water (2.13 mL) were added successively into the reactant and the reaction was stopped. Magnesium sulfate anhydrous was added thereto, and resulting mixture was stirred for 20 minutes, then filtrated and purified by column chromatography with ethyl acetate to give the title compound (2.9 mg, 10%).

¹H NMR (400 MHz, CDCl₃); δ 10.02 (1H, s), 7.95 (2H, d), 7.34 (2H, d), 7.01 (1H, s), 5.31 (2H, s), 4.28 (2H, m), 4.24 (2H, m), 2.86 (2H, t), 1.77 (2H, m), 1.02 (3H, t)

### Example 3-48

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-phenyl}-methanol

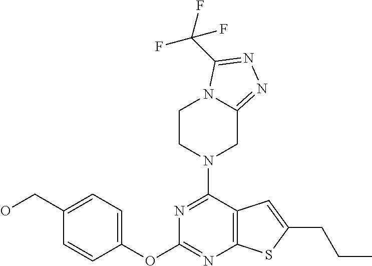

The compound obtained from Example 3-35 (30 mg, 0.059 mmol) was reacted according to the same method as Example 3-47 to give the title compound (3.8 mg, 13%).

¹H NMR (400 MHz, CDCl₃); δ 7.40 (2H, d), 7.16 (2H, d), 6.96 (1H, s), 5.25 (2H, s), 4.74 (2H, s), 4.24 (2H, m), 4.20 (2H, m), 2.84 (2H, t), 1.74 (2H, m), 1.01 (3H, t)

### Preparation Example 3-49-1

### 2-Chloro-6-ethyl-3H-thieno[2,3-d]pyrimidin-4-one

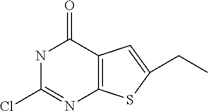

The compound obtained from Preparation Example 2-2-1 (500 mg, 2.14 mmol) was dissolved in the mixture (20 mL) of tetrahydrofuran and water (1:1), potassium hydroxide 488 mg (8.70 mmol) was added thereto and then stirred for 20 hours. The resulting mixture was distilled under reduced pressure to remove tetrahydrofuran and acidified with aqueous 1 N HCl (pH=4). Resulting solid was filtrated, washed with water and then dried to give the title compound (450 mg, 98%).

¹H NMR (500 MHz, CDCl₃); δ 11.93 (1H, br s), 7.18 (1H, s), 2.88 (2H, q), 1.36 (3H, t)

### Preparation Example 3-49-2

### 2-Allyloxy-6-ethyl-3H-thieno[2,3-d]pyrimidin-4-one

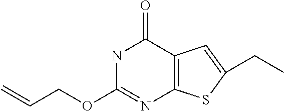

Anhydrous allyl alcohol (25 mL) was cooled to 0° C. and sodium (555 mg, 24.1 mmol) was cut and added thereto. After the sodium was completely dissolved, the compound obtained from Preparation Example 3-49-1 (690 mg, 3.21 mmol) was added thereto and then stirred under reflux for 12 hours. The resulting mixture was acidified with aqueous 1 N HCl (pH=4). Water 50 mL was added thereto and extracted twice with ethyl acetate 100 mL. Organic layer was dried with anhydrous magnesium sulfate and distilled under reduced pressure to give the title compound (750 mg, 98%).

¹H NMR (500 MHz, CDCl₃); δ 9.60 (1H, br s), 7.05 (1H, s), 6.03 (1H, m), 5.44 (1H, d), 5.32 (1H, d), 4.91 (2H, d), 2.82 (2H, q), 1.32 (3H, t)

### Preparation Example 3-49-3

### 2-(2,3-Dihydroxy-propoxy)-6-ethyl-3H-thieno[2,3-d]pyrimidin-4-one

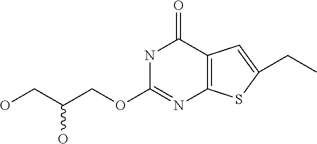

The compound obtained from Preparation Example 3-49-2 (57 mg, 0.24 mmol) was dissolved in the 1:1 mixture of tetrahydrofuran and water (6 mL), 4-methyl morpholine N-oxide (37 mg, 0.32 mmol) and osmium tetroxide (2.5 mg, 2.5 wt. % in t-butanol 100 mg, 0.01 mmol) was added thereto and then stirred for 3 hours. Ethyl acetate (15 mL) was added thereto and the resulting mixture was washed twice with water (5 mL). Organic layer was dried with anhydrous magnesium sulfate, distilled under reduced pressure, then purified by column chromatography with the 1:1 mixture of hexane and ethyl acetate to give the title compound (45 mg, 69%).

¹H NMR (500 MHz, CDCl₃); δ 6.97 (1H, s), 4.40 (2H, m), 3.97 (1H, m), 3.61 (2H, m), 3.35 (2H, m), 2.77 (2H, q), 1.27 (3H, t)

### Preparation Example 3-49-4

### Acetic Acid

### 1-acetoxymethyl-2-(6-ethyl-4-oxo-3,4-dihydro-thieno[2,3-d]pyrimidin-2-yloxy)-ethyl ester

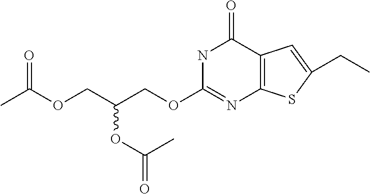

The compound obtained from Preparation Example 3-49-3 (45 mg, 0.17 mmol) was dissolved in N,N-dimethylformamide (4 mL), pyridine (0.2 mL, 2.47 mmol) and acetic anhydride (0.1 mL, 1.06 mmol) were added thereto and stirred for 12 hours. The resulting mixture was distilled under reduced pressure to remove N,N-dimethylformamide, ethyl acetate (15 mL) was added thereto and washed twice with water (5 mL). Organic layer was dried with anhydrous magnesium sulfate, distilled under reduced pressure and then purified by column chromatography with the 1:1 mixture of hexane and ethyl acetate to give the title compound (58 mg, 99%).

¹H NMR (500 MHz, CDCl₃); δ 11.14 (1H, br s), 7.04 (1H, s), 5.40 (1H, m), 4.67 (1H, dd), 4.48 (1H, dd), 4.39 (1H, dd), 4.24 (1H, dd), 2.82 (2H, q), 2.10 (6H, s), 1.31 (3H, t)

### Preparation Example 3-49-5

### Acetic acid 2-acetoxy-3-(4-chloro-6-ethyl-thieno[2,3-d]pyrimidin-2-yloxy)-propyl ester

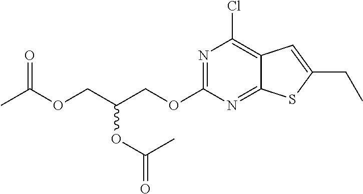

The compound obtained from Preparation Example 3-49-4 (58 mg, 0.16 mmol) was dissolved in acetonitrile (3 mL), N,N-dimethylaniline (0.02 mL, 0.16 mmol) and phosphorous oxychloride (0.09 mL, 0.98 mmol) were added thereto and then stirred at 70° C. for 12 hours. The resulting mixture was distilled under reduced pressure and then purified by column chromatography with the 5:1 mixture of hexane and ethyl acetate to give the title compound (51 mg, 82%).

¹H NMR (500 MHz, CDCl₃); δ 6.97 (1H, s), 5.40 (1H, m), 4.65 (1H, dd), 4.54 (1H, dd), 4.45 (1H, dd), 4.30 (1H, dd), 2.90 (2H, q), 2.08 (6H, s), 1.38 (3H, t)

### Preparation Example 3-49-6

### Acetic Acid

### 2-acetoxy-3-[6-ethyl-4-(3-trifluoromethyl-5,6-dihydro-8H-[1,2,4]triazolo[4,3-a]pyrazin-7-yl)-thieno[2,3-d]pyrimidin-2-yloxy]-propyl ester

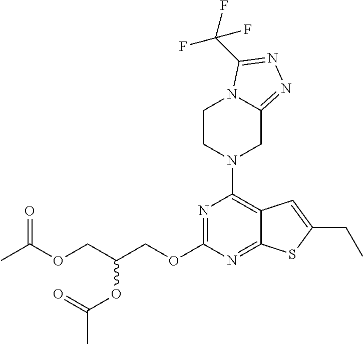

The compound obtained from Preparation Example 3-49-5 (28 mg, 0.075 mmol) and the compound obtained from Preparation Example 1-1-2 (21 mg, 0.09 mmol) were diluted in N,N-dimethylformamide (5 mL), then diisopropylethylamine (29 mg, 0.225 mmol) was added and stirred for 16 hours at 80° C. The resulting mixture was distilled under reduced pressure, then diluted with dichloromethane and washed with water. Organic layer was dried with anhydrous magnesium sulfate, distilled under reduced pressure and then purified by column chromatography with the 1:1 mixture of hexane and ethyl acetate to give the title compound (34 mg, 85%).

¹H NMR (400 MHz, CDCl₃); δ 6.95 (1H, s), 5.43 (1H, m), 5.32 (2H, s), 4.59˜4.26 (8H, m), 2.90 (2H, q), 2.10 (3H, s), 2.09 (3H, s), 1.37 (3H, t)

### Example 3-49

### 3-[6-Ethyl-4-(3-trifluoromethyl-5,6-dihydro-8H-[1,2,4]triazolo[4,3-a]pyrazin-7-yl)-thieno[2,3-d]pyrimidin-2-yloxy]-propane-1,2-diol

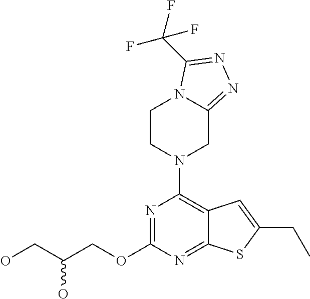

The compound obtained from Preparation Example 3-49-6 (11 mg, 0.022 mmol) was dissolved in tetrahydrofuran (3 mL) and methanol (0.5 mL), then 1.0 N aqueous sodium hydroxide solution (0.066 mL, 0.066 mmol) was added thereto and stirred for 1 hours. The resulting mixture was acidified with 1.0 N aqueous hydrochloric acid solution and distilled under reduced pressure to remove the solvent and then purified by column chromatography with the 15:85 mixture of methanol and dichloromethane to give the title compound (6 mg, 67%).

¹H NMR (400 MHz, CDCl₃); δ 6.95 (1H, s), 5.31 (2H, s), 4.49 (2H, m), 4.35 (4H, m), 4.12 (1H, m), 3.76 (2H, m), 3.37 (1H, br s), 2.90 (2H, q), 2.62 (1H, br s), 1.37 (3H, t)

### Preparation Example 3-50-1

### 6-Ethyl-2-(2-hydroxy-ethoxy)-3H-thieno[2,3-d]pyrimidin-4-one

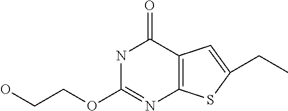

The compound obtained from Preparation Example 3-49-3 (30 mg, 0.11 mmol) was dissolved in the 1:1 mixture of tetrahydrofuran and water (3 mL), then sodium periodate (32 mg, 0.15 mmol) was added thereto and stirred for 3 hours. The resulting mixture was distilled under reduced pressure, then water was added thereto and was extracted twice with ethyl acetate (15 mL). Organic layer was dried with anhydrous magnesium sulfate and distilled under reduced pressure. The concentrate was dissolved in methanol (3 mL), sodium borohydride (13 mg, 0.33 mmol) was added thereto and then stirred for 30 minutes. 1N aqueous hydrochloric acid was added thereto, distilled under reduced pressure to remove methanol and water was added thereto. The resulting mixture was extracted twice by ethyl acetate (10 mL), then organic layer was dried with anhydrous magnesium sulfate and distilled under reduced pressure to give the title compound (24 mg, 90%).

¹H NMR (500 MHz, DMSO, d₆); δ 12.29 (1H, br s), 6.92 (1H, s), 4.86 (1H, br s), 4.32 (2H, t), 3.65 (2H, m), 2.75 (2H, q), 1.20 (3H, t)

### Preparation Example 3-50-2

### Acetic acid 2-(6-ethyl-4-oxo-3,4-dihydro-thieno[2,3-d]pyrimidin-2-yloxy)-ethyl ester

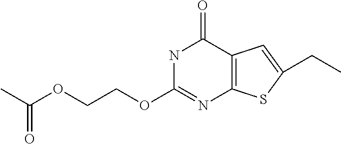

The compound obtained from Preparation Example 3-50-1 (24 mg, 0.01 mmol) was dissolved in N,N-dimethylformamide (2 mL), and pyridine (0.1 mL, 0.12 mmol) and acetic anhydride (0.05 mL, 0.06 mmol) was added thereto and then stirred for 12 hours. The resulting mixture was distilled under reduced pressure to remove N,N-dimethylformamide, ethyl acetate (10 mL) was added thereto and washed twice with water (5 mL). Organic layer was dried with anhydrous magnesium sulfate and distilled under reduced pressure to give the title compound (28 mg, 99%).

¹H NMR (500 MHz, CDCl₃); δ 9.28 (1H, br s), 7.05 (1H, s), 4.62 (1H, t), 4.41 (2H, t), 2.82 (2H, q), 2.09 (3H, s), 1.32 (3H, t)

### Preparation Example 3-50-3

### Acetic acid 2-(4-chloro-6-ethyl-thieno[2,3-d]pyrimidin-2-yloxy)-ethyl ester

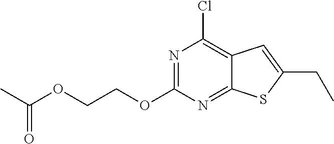

The compound obtained from Preparation Example 3-50-2 (28 mg, 0.01 mmol) was dissolved in acetonitrile (2 mL), N,N-dimethylaniline (0.01 mL, 0.01 mmol) and phosphorous oxychloride (0.03 mL, 0.04 mmol) were added thereto and then stirred for 12 hours at 70° C. The resulting mixture was distilled under reduced pressure and then purified by column chromatography with the 5:1 mixture of hexane and ethyl acetate to give the title compound (27 mg, 90%).

¹H NMR (500 MHz, CDCl₃); δ 6.95 (1H, s), 4.62 (1H, t), 4.43 (2H, t), 2.89 (2H, q), 2.07 (3H, s), 1.37 (3H, t)

### Preparation Example 3-50-4

### Acetic acid 2-[6-ethyl-4-(3-trifluoromethyl-5,6-dihydro-8H-[1,2,4]triazolo[4,3-a]pyrazin-7-yl)-thieno[2,3-d]pyrimidin-2-yloxy]-ethyl ester

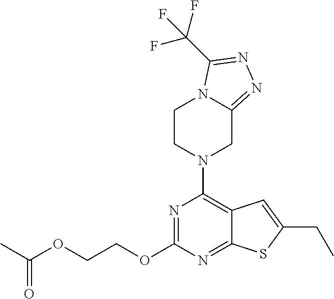

The compound obtained from Preparation Example 3-50-3 (27 mg, 0.09 mmol) and the compound obtained from Preparation Example 1-1-2 (26 mg, 0.135 mmol) were distilled in N,N-dimethylformamide (5 mL), then diisopropylethylamine (23 mg, 0.18 mmol) was added thereto, heated to 150° C. in a microwave reactor and stirred for 2 hours. The resulting mixture was cooled to the room temperature, distilled under reduced pressure and then purified by column chromatography with the 5:95 mixture of methanol and dichloromethane to give the title compound (18 mg, 44%).

¹H NMR (400 MHz, CDCl₃); δ 6.95 (1H, s), 5.31 (2H, s), 4.60 (2H, dd), 4.44 (2H, dd), 4.35 (4H, m), 2.90 (2H, q), 2.09 (3H, s), 1.37 (3H, t)

### Example 3-50

### 2-[6-Ethyl-4-(3-trifluoromethyl-5,6-dihydro-8H-[1,2,4]triazolo[4,3-a]pyrazin-7-yl)-thieno[2,3-d]pyrimidin-2-yloxy]-ethanol

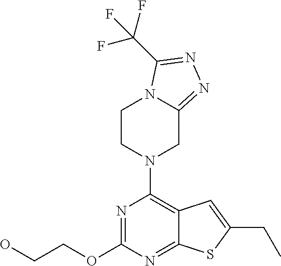

The compound obtained from Preparation Example 3-50-4 (18 mg, 0.039 mmol) was dissolved in tetrahydrofuran (3 mL) and methanol (0.5 mL), and then 1.0 N aqueous sodium hydroxide solution (0.078 mL, 0.078 mmol) was added thereto and stirred for 1 hours. The resulting mixture was acidified with 1.0 N aqueous hydrochloric acid solution, distilled under reduced pressure to remove the solvent, and then purified by column chromatography with the 7:93 mixture of methanol and dichloromethane to give the title compound (15 mg, 94%).

¹H NMR (400 MHz, CDCl₃); δ 6.95 (1H, s), 5.31 (2H, s), 4.52 (2H, dd), 4.37 (2H, dd), 4.32 (2H, dd), 3.99 (2H, dd), 2.90 (2H, q), 2.65 (1H, t), 1.37 (3H, t)

### Example 3-51

### 7-(2-Benzylsulfanyl-6-propyl-thieno[2,3-d]pyrimidin-4-yl)-3-trifluoromethyl-5,6,7,8-tetrahydro-[1,2,4]triazolo[4,3-a]pyrazine

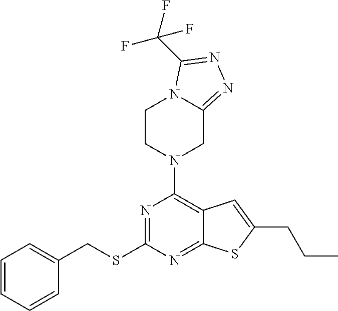

The compound obtained from Preparation Example 1-1-3 (40 mg, 0.1 mmol) and phenyl-methanthiol (19 mg, 0.15 mmol) were dissolved in N,N-dimethylformamide (2 mL), DBU (30 mg, 0.2 mmol) was added thereto and stirred for 16 hours. The resulting mixture was distilled under reduced pressure to remove the solvent, diluted with ethyl acetate and washed with water and salt water. Organic layer was dried with anhydrous magnesium sulfate, distilled under reduced pressure and then purified by column chromatography with the 1:1 mixture of hexane and ethyl acetate to give the title compound (16 mg, 33%).

¹H NMR (500 MHz, CDCl₃); δ 7.44 (2H, d), 7.30 (2H, t), 7.23 (1H, t), 6.92 (1H, s), 5.24 (2H, s), 4.42 (2H, s), 4.20 (2H, s), 2.84 (2H, t), 1.75 (2H, m), 1.01 (3H, t)

### Example 3-52

### 7-(2-Phenylsulfanyl-6-propyl-thieno[2,3-d]pyrimidin-4-yl)-3-trifluoromethyl-5,6,7,8-tetrahydro-[1,2,4]triazolo[4,3-a]pyrazine

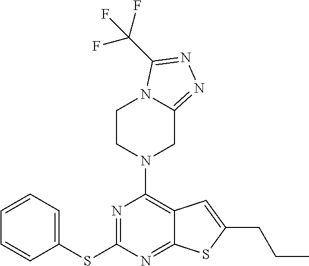

The compound obtained from Preparation Example 1-1-3 (50 mg, 0.124 mmol), benzenethiol (27 mg, 0.248 mmol) and DBU 56 uL (0.372 mmol) were reacted according to the similar method to Example 1-1 to give the title compound (17 mg, 29%).

¹H NMR (400 MHz, CDCl₃); δ 7.74 (2H, d), 7.35˜7.32 (3H, m), 6.61 (1H, s), 4.55 (2H, s), 3.92 (2H, t), 3.16 (2H, t), 2.64 (2H, t), 1.63 (2H, m), 1.01 (3H, t)

### Example 3-53

### 7-[6-Propyl-2-(pyrimidin-2-ylsulfanyl)-thieno[2,3-d]pyrimidin-4-yl]-3-trifluoromethyl-5,6,7,8-tetrahydro-[1,2,4]triazolo[4,3-a]pyrazine

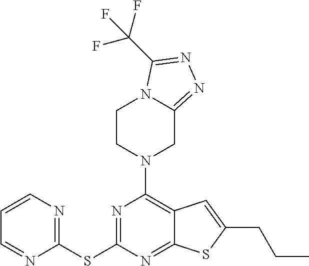

The compound obtained from Preparation Example 1-1-3 (50 mg, 0.124 mmol), pyrimidin-2-thiol (27 mg, 0.248 mmol) and DBU (56 uL, 0.372 mmol) were reacted according to the similar method to Example 1-1 to give the title compound (9 mg, 15%).

¹H NMR (400 MHz, CDCl₃); δ 8.61 (2H, d), 7.15 (1H, t), 7.01 (1H, s), 5.26 (2H, s), 4.28 (2H, m), 4.22 (2H, m), 2.89 (2H, t), 1.77 (2H, m), 1.01 (3H, t)

### Preparation Example 3-54-1

### [tert-Butoxycarbonyl-(2-hydroxy-ethyl)-amino]-acetic acid ethyl ester

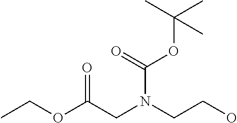

2-aminoethanol (5.0 g, 81.85 mmol) was dissolved in tetrahydrofuran (50 mL), ethyl bromoacetate (11.78 mL, 106.40 mmol) and triethylamine (17.11 mL, 122.77 mmol) were added thereto at 0° C., and then stirred at room temperature for 48 hours. The resulting mixture was filtrated and the filtrate was washed with tetrahydrofuran (20 mL) and then combined with initial filtrate. Di t-butyl dicarbonate (17.86 g, 81.85 mmol) was added to the combined solution and distilled under reduced pressure at 50° C. to complete the reaction. Toluene (200 mL) was added threrto, resulting mixture was washed with 1N aqueous hydrochloric acid solution, saturated aqueous sodium hydrogen carbonate solution and salt water. The resulting mixture was distilled under reduced pressure and purified by column chromatography with the 1:1 mixture of ethyl acetate and hexane to give the title compound (6.6 g, 33%).

¹H NMR (400 MHz, CDCl₃); δ 4.24 (2H, q), 3.92˜3.97 (2H, m), 3.68˜3.77 (2H, m), 3.36˜3.47 (3H, m), 1.43˜1.47 (9H, m), 1.29 (3H, t)

### Preparation Example 3-54-2

### (tert-Butoxycarbonyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-acetic acid ethyl ester

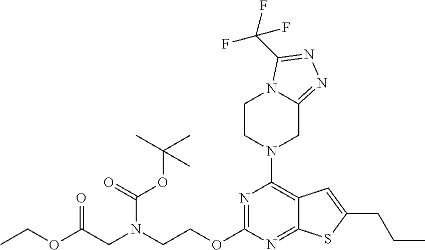

The compound obtained from Preparation Example 1-1-3 (300 mg, 0.745 mmol) and the compound obtained from Preparation Example 3-54-1 (276 mg, 1.120 mmol) were reacted according to the similat method to Example 1-45 to give the title compound (190 mg, 42%).

¹H NMR (400 MHz, CDCl₃); δ 6.94 (1H, s), 5.31 (2H, m), 4.48˜4.52 (2H, m), 4.29˜4.38 (4H, m), 4.04˜4.18 (4H, m), 3.70 (2H, m), 2.85 (2H, m), 1.74 (2H, m), 1.42˜1.62 (9H, m), 1.28 (3H, t)

### Example 3-54

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethylamino}-acetic acid ethyl ester

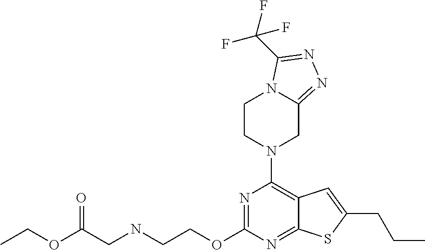

The compound obtained from Preparation Example 3-54-2 (145 mg, 0.236 mmol) was reacted according to the similar method to Example 1-4 to give the title compound (100 mg, 77%).

¹H NMR (400 MHz, DMSO, d₆); δ 9.41 (2H, br s), 7.45 (1H, s), 5.24 (2H, s), 4.60 (2H, m), 4.36 (2H, m), 4.31 (2H, m), 4.20 (2H, q), 4.06 (2H, m), 3.42 (2H, m), 2.85 (2H, t), 1.68 (2H, m), 1.24 (3H, t), 0.96 (3H, t)

### Preparation Example 3-55

### (tert-Butoxycarbonyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-acetic acid

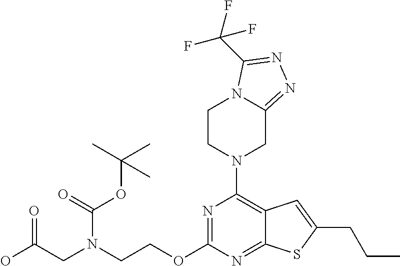

The compound obtained from Preparation Example 3-54-2 (45 mg, 0.073 mmol) was dissolved in the 5:3:1 mixture of tetrahydrofuran, water and methanol, lithium hydroxide (0.15 mg, 0.147 mmol) was added thereto and the resulting mixture was stirred at room temperature for 2 hours. The resulting mixture was distilled under reduced pressure and then purified by column chromatography with the 1:1 mixture of methanol and dichloromethane to give the title compound (20 mg, 47%).

¹H NMR (400 MHz, CDCl₃); δ 6.94 (1H, s), 5.30 (2H, s), 4.51 (2H, m), 4.36 (2H, m), 4.32 (2H, m), 4.11 (2H, m), 3.70 (2H, m), 2.82 (2H, t), 1.73 (2H, m), 1.43 (9H, m), 1.01 (3H, t)

### Example 3-55

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethylamino}-acetic acid

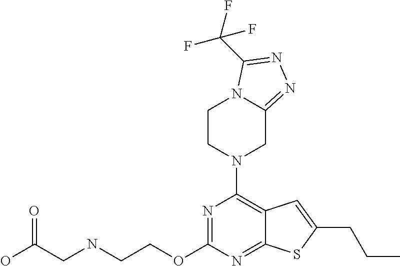

The compound obtained from Preparation Example 3-55 (20 mg, 0.034 mmol) was reacted according to the similar method to Example 1-4 to give the title compound (15 mg, 84%).

¹H NMR (400 MHz, DMSO, d₆); δ 9.21 (2H, br), 7.44 (1H, s), 5.24 (2H, s), 4.59 (2H, m), 4.37 (2H, m), 4.31 (2H, m), 3.96 (2H, m), 3.41 (2H, m), 2.83 (2H, t), 1.68 (2H, m), 0.96 (3H, t)

### Example 3-56

### (Cyclopropanecarbonyl-{2-[6-propyl-4-(3-trifluoromethyl-5,6-dihydro-8H-[1,2,4]triazolo[4,3-a]pyrazin-7-yl)-thieno[2,3-c]pyrimidin-2-yloxy]-ethyl}-amino)-acetic acid ethyl ester

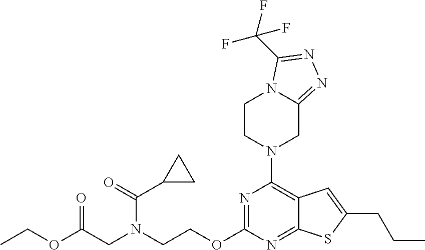

The compound obtained from Example 3-54 (50 mg, 0.091 mmol) and cyclopropane carboxylic acid (8.69 uL, 0.109 mmol) were dissolved in N,N-dimethylformamide (5 mL) and then HBTU (52 mg, 0.136 mmol) was added thereto. The resulting mixture was cooled to 0° C., diisopropylethylamine (47.46 uL, 0.272 mmol) was added dropwise thereto and then stirred at room temperature for 4 hours. The resulting mixture was diluted with ethyl acetate and washed with water and salt water. Organic layer was dried with anhydrous magnesium sulfate, distilled under reduced pressure and then purified by column chromatography with the 2:1 mixture of ethyl acetate and hexane to give the title compound (30 mg, 57%).

¹H NMR (400 MHz, CDCl₃); δ 6.95 (1H, m), 5.30 (2H, s), 4.61 (1.2H, t), 4.50 (0.8H, 0.8H), 4.41 (0.8H, s), 4.36 (2H, m), 4.31 (2H, m), 4.13˜4.19 (3.2H, m), 3.98 (1.2H, t), 3.84 (0.8H, t), 2.83 (2H, m), 1.99 (0.6H, m), 1.76 (2H, m), 1.52 (0.4H, m), 1.26 (3H, m), 1.01 (5H, m), 0.84 (1.2H, m), 0.75 (0.8H, m)

### Example 3-57

### (Cyclopropanecarbonyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-acetic acid

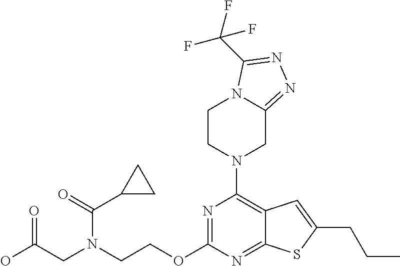

The compound obtained from Example 3-56 (30 mg, 0.052 mmol) and lithium hydroxide (4.33 mg, 0.103 mmol) were reacted according to the similar method to Preparation Example 3-55 to give the title compound (12 mg, 42%).

¹H NMR (400 MHz, CDCl₃); δ 6.94 (1H, s), 5.28 (2H, s), 4.60 (1.2H, m), 4.21˜4.46 (6.8H, m), 3.99 (1.2H, m), 3.84 (0.8H, m), 2.80 (2H, m), 1.84 (0.6H, m), 1.73 (2H, m), 1.59 (0.4H, m), 1.00 (5H, m), 0.77˜0.88 (2H, m)

### Preparation Example 3-58

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid tert-butyl ester

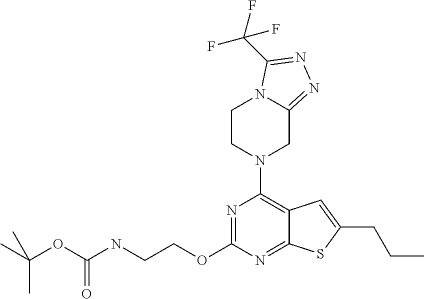

The compound obtained from Preparation Example 1-1-3 (100 mg, 0.248 mmol) and (2-hydroxyethyl)-carbamic acid t-butyl ester (80 mg, 0.496 mmol) were reacted according to the similar method to Example 1-45 to give the title compound (100 mg, 76%).

¹H NMR (400 MHz, CDCl₃); δ 6.95 (1H, s), 5.31 (2H, s), 5.06 (1H, br s), 4.42 (2H, t), 4.38 (2H, m), 4.33 (2H, m), 3.55 (2H, m), 2.83 (2H, t), 1.76 (2H, m), 1.01 (3H, t)

### Example 3-58

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethylamine

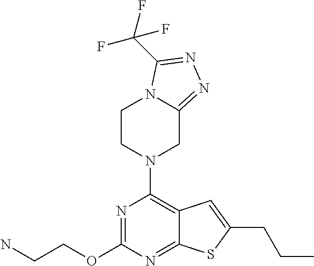

The compound obtained from Preparation Example 3-58 (140 mg, 0.265 mmol) was reacted according to the similar method to Example 1-4 to give the title compound (126 mg, 102%).

¹H NMR (400 MHz, DMSO, d₆); δ 8.18 (3H, br s), 7.44 (1H, s), 5.23 (2H, s), 4.05 (2H, m), 4.36 (2H, m), 4.31 (2H, m), 3.22 (2H, m), 2.85 (2H, t), 1.68 (2H, m), 0.96 (3H, t)

### Example 3-59

### Cyclopropanecarboxylic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

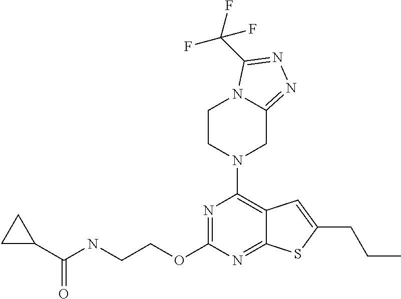

The compound obtained from Example 3-58 (50 mg, 0.108 mmol) and cyclopropane carboxylic acid (10.3 uL, 0.129 mmol) were reacted according to the similar method to Example 3-56 to give the title compound (40 mg, 75%).

¹H NMR (400 MHz, CDCl₃); δ 6.95 (1H, s), 6.21 (1H, br s), 5.31 (2H, s), 4.46 (2H, t), 4.37 (2H, m), 4.33 (2H, m), 3.71 (2H, m), 2.86 (2H, t), 1.74 (2H, m), 1.35 (1H, m), 1.01 (3H, t), 0.95 (2H, m), 0.76 (2H, m)

### Example 3-60

### 2-Hydroxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

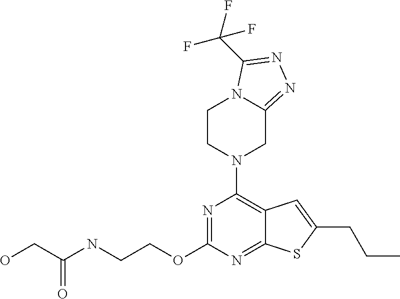

The compound obtained from Example 3-58 (50 mg, 0.108 mmol) and hydroxyacetic acid (9.84 mg, 0.129 mmol) were reacted according to the similar method to Example 3-56 to give the title compound (37 mg, 71%).

¹H NMR (500 MHz, CDCl₃); δ 6.97 (1H, br s), 6.93 (1H, s), 5.30 (2H, s), 4.49 (2H, t), 4.36 (2H, m), 4.32 (2H, m), 4.12 (2H, d), 3.76 (2H, q), 2.83 (2H, t), 2.52 (1H, t), 1.75 (2H, m), 1.00 (3H, t)

### Example 3-61

### 2,2,2-Trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

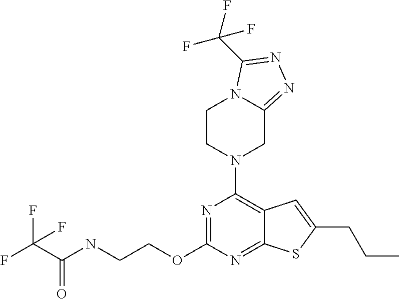

The compound obtained from Example 3-58 (50 mg, 0.108 mmol) and trifluoroacetic anhydride (30.0 uL, 0.216 mmol) were reacted according to the similar method to Example 3-56 to give the title compound (30 mg, 53%).

¹H NMR (400 MHz, CDCl₃); δ 7.43 (1H, br s), 6.97 (1H, s), 5.33 (2H, s), 4.57 (2H, t), 4.37 (2H, m), 4.34 (2H, m), 3.79 (2H, m), 2.85 (2H, t), 1.75 (2H, m), 1.02 (3H, t)

### Example 3-62

### 1-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-pyrrolidin-2-one

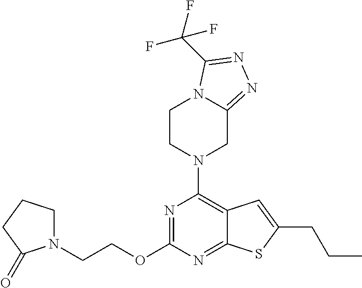

The compound obtained from Preparation Example 1-1-3 (50 mg, 0.124 mmol) and 1-(2-hydroxyethyl)-pyrrolidine-2-on (32 mg, 0.248 mmol) were reacted according to the similar method to Example 1-45 to give the title compound (28 mg, 46%).

¹H NMR (400 MHz, CDCl₃); δ 6.94 (1H, s), 5.29 (1H, br s), 4.51 (2H, t), 4.33˜4.37 (4H, m), 3.70 (2H, t), 3.58 (2H, t), 2.83 (2H, t), 2.37 (2H, t), 2.02 (2H, m), 1.76 (2H, m), 1.01 (3H, t)

### Example 3-63

### 2-Methoxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

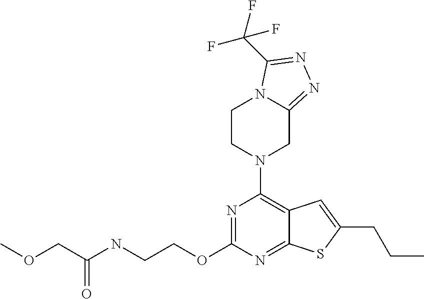

The compound obtained from Example 3-58 (50 mg, 0.108 mmol) and methoxy acetic acid (9.92 uL, 0.129 mmol) were reacted according to the similar method to Example 3-56 to give the title compound (20 mg, 37%).

¹H NMR (400 MHz, CDCl₃); δ 7.10 (1H, br s), 6.95 (1H, s), 5.31 (2H, s), 4.49 (2H, t), 4.37 (2H, m), 4.32 (2H, m), 3.90 (2H, s), 3.75 (2H, q), 3.39 (3H, s), 2.84 (2H, t), 1.76 (2H, m), 1.01 (3H, t)

### Example 3-64

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-methanesulfonamide

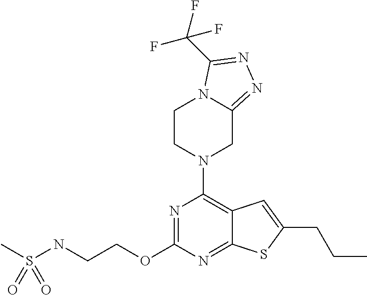

The compound obtained from Example 3-58 (50 mg, 0.108 mmol) and diisopropylethylamine (56 uL, 0.323 mmol) were dissolved in dichloromethane (5 mL), methanesulphonyl chloride (10.01 uL, 0.129 mmol) was added thereto at 0° C. and stirred at room temperature for 6 hours. The resulting mixture was washed with saturated aqueous sodium hydrogen carbonate, dried with anhydrous magnesium sulfate and distilled under reduced pressure. The resulting mixture was purified by column chromatography with the 20:1 mixture of dichloromethane and methanol to give the title compound (30 mg, 55%).

¹H NMR (400 MHz, DMSO, d₆); δ 7.41 (1H, s), 7.28 (1H, br s), 5.22 (2H, s), 4.35 (4H, m), 4.29 (2H, m), 3.34 (2H, m), 2.95 (3H, s), 2.84 (2H, t), 1.68 (2H, m), 0.96 (3H, t)

### Preparation Example 3-65

### ({2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethylcarbamoyl}-methyl)-carbamic acid tert-butyl ester

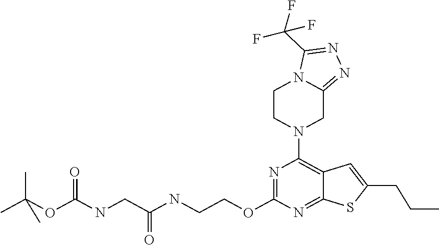

The compound obtained from Example 3-58 (50 mg, 0.108 mmol) and t-butoxycarbonylamino acetic acid (22.66 mg, 0.129 mmol) were reacted according to the similar method to Example 3-56 to give the title compound (42 mg, 67%).

¹H NMR (400 MHz, CDCl₃); δ 6.95 (1H, s), 6.51 (1H, br s), 5.31 (2H, s), 5.01 (1H, br s), 4.47 (2H, t), 4.37 (2H, m), 4.33 (2H, m), 3.79 (2H, d), 3.73 (2H, q), 2.83 (2H, t), 1.76 (2H, m), 1.42 (9H, s), 1.01 (3H, t)

### Example 3-65

### 2-Amino-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

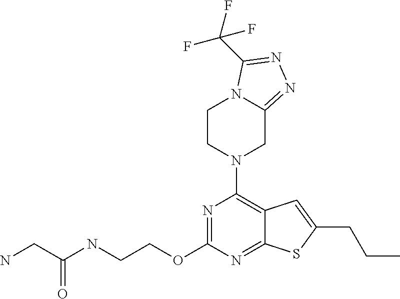

The compound obtained from Preparation Example 3-65 (42 mg, 0.718 mmol) was reacted according to the similar method to Example 1-4 to give the title compound (30 mg, 80%).

¹H NMR (400 MHz, DeOD); δ 7.42 (1H, s), 5.46 (2H, s), 4.60 (2H, m), 4.50 (4H, m), 3.71 (4H, m), 2.93 (2H, t), 1.76 (2H, m), 1.02 (3H, t)

### Example 3-66

### 2-Methanesulfonyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

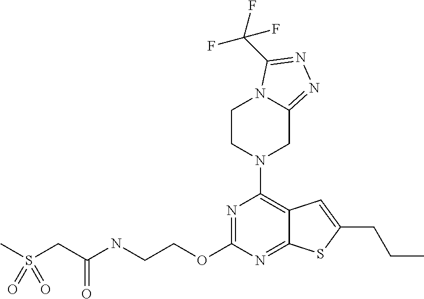

The compound obtained from Example 3-58 (50 mg, 0.108 mmol) and methanesulphonyl acetic acid (17.87 mg, 0.129 mmol) were reacted according to the similar method to Example 3-56 to give the title compound (26 mg, 44%).

¹H NMR (400 MHz, CDCl₃); δ 6.95 (1H, s), 6.85 (1H, br s), 5.31 (2H, s), 4.52 (2H, t), 4.36 (2H, m), 4.32 (2H, m), 3.85 (2H, s), 3.75 (2H, q), 3.08 (3H, s), 2.84 (2H, t), 1.76 (2H, m), 1.01 (3H, t)

### Example 3-67

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-4-sulfamoyl-butyramide

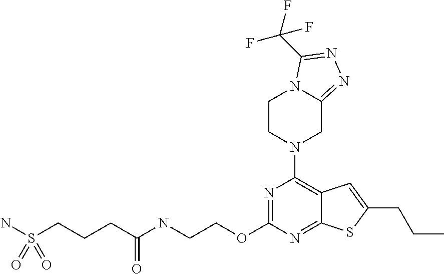

The compound obtained from Example 3-58 (50 mg, 0.108 mmol) and 4-sulfamoylbutyric acid (21.66 mg, 0.129 mmol) were reacted according to the similar method to Example 3-56 to give the title compound (30 mg, 48%).

¹H NMR (400 MHz, CDCl₃); δ 6.94 (1H, s), 6.39 (1H, br s), 5.28 (2H, s), 4.49 (2H, t), 4.36 (2H, m), 4.32 (2H, m), 3.66 (2H, q), 3.21 (2H, t), 2.83 (2H, t), 2.45 (2H, t), 2.22 (2H, m), 1.76 (2H, m), 1.01 (3H, t)

### Example 3-68

### Cyclopropanesulfonic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

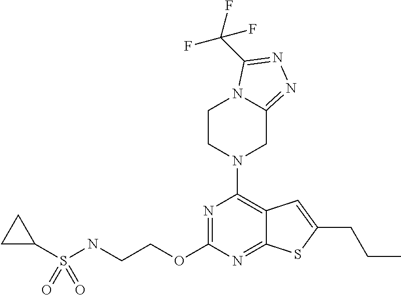

The compound obtained from Example 3-58 (50 mg, 0.108 mmol) and cyclopropanesulfonyl chloride (13 uL, 0.129 mmol) were reacted according to the similar method to Example 3-64 to give the title compound (33 mg, 58%).

¹H NMR (400 MHz, CDCl₃); δ 6.96 (1H, s), 5.31 (2H, s), 4.87 (1H, t), 4.53 (2H, t), 4.37 (2H, m), 4.33 (2H, m), 3.58 (2H, q), 2.84 (2H, t), 2.46 (1H, m), 1.75 (2H, m), 1.19 (2H, m), 1.01 (5H, m)

### Example 3-69

### C,C,C-Trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-methanesulfonamide

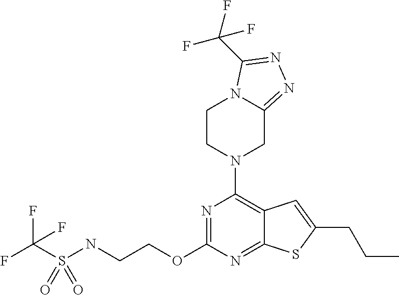

The compound obtained from Example 3-58 (50 mg, 0.108 mmol) and trifluoromethanesulphonyl chloride (13.77 uL, 0.129 mmol) were reacted according to the similar method to Example 3-64 to give the title compound (25 mg, 41%).

¹H NMR (400 MHz, CDCl₃); δ 6.97 (1H, s), 5.96 (1H, br s), 5.32 (2H, s), 4.55 (2H, t), 4.37 (2H, m), 4.33 (2H, m), 3.72 (2H, m), 2.84 (2H, t), 1.75 (2H, m), 1.19 (2H, m), 1.02 (3H, t)

### Example 3-70

### Pyridine-2-carboxylic acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

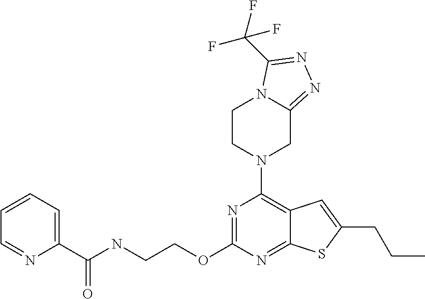

The compound obtained from Example 3-58 (50 mg, 0.108 mmol) and pyridine-2-carboxylic acid (15.92 mg, 0.129 mmol) were reacted according to the similar method to Example 3-56 to give the title compound (38 mg, 66%).

¹H NMR (400 MHz, CDCl₃); δ 8.51 (1H, br s), 8.51 (1H, d), 8.16 (1H, d), 7.82 (1H, m), 7.40 (1H, m), 6.94 (1H, s), 5.30 (2H, s), 4.60 (1H, t), 4.53 (2H, t), 4.37 (2H, m), 4.33 (2H, m), 3.92 (2H, q), 2.84 (2H, t), 1.76 (2H, m), 1.01 (3H, t)

### Preparation Example 4-1-1

### 2-Amino-5-propyl-thiophene-3-carboxylic acid methyl ester

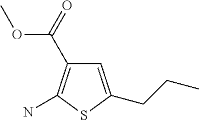

Methyl cyanoacetate (40.0 g, 404 mmol), sulfur (12.9 g, 404 mmol) and triethylamine (28.2 mL, 202 mmol) were dissolved in N,N-dimethylformamide (200 mL) and pentanal (35.0 g, 404 mmol) was slowly added dropwise thereto maintaining the reaction temperature 50° C. Ethyl acetate (200 mL) was added thereto and the resulting mixture was washed twice with water (200 mL). Organic layer was dried with anhydrous magnesium sulfate, distilled under reduced pressure and then purified by column chromatography with the 5:1 mixture of hexane and ethyl acetate to give the title compound (51.8 g, 64%).

¹H NMR (500 MHz, CDCl₃); δ 6.61 (1H, s), 5.77 (1H, br s), 3.78 (3H, s), 2.52 (3H, t), 1.58 (2H, m), 0.94 (3H, s)

### Preparation Example 4-1-2

### 4-Oxo-6-propyl-3,4-dihydrothieno[2,3-d]pyrimidine-2-carboxylic acid ethyl ester

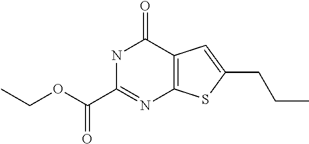

The compound obtained from Preparation Example 4-1-1 (10 g, 50.18 mmol) and ethyl cyanoformate (4.96 g, 50.18 mmol) were dissolved in dioxane (20 mL) and then cooled to 0° C. 4.0 M hydrochloric acid dioxane solution (100 mL) was slowly added dropwise and stirred ar room temperature for 16 hours. The resulting mixture was distilled under reduced pressure to remove the solvent, diluted with water, and then basified with saturated aqueous sodium hydrogen carbonate solution. Thus obtained solid compound was washed several times with hexane to give the title compound (8 g, 59%).

¹H NMR (400 MHz, CDCl₃); δ 10.22 (1H, br s), 7.28 (1H, s), 4.56 (2H, q), 2.87 (2H, t), 1.77 (2H, m), 1.48 (3H, t), 1.01 (3H, t)

### Preparation Example 4-1-3

### 4-Chloro-6-propyl-thieno[2,3-d]pyrimidine-2-carboxylic acid ethyl ester

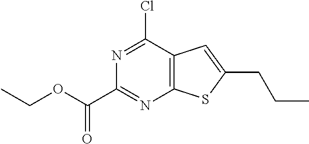

The compound obtained from Preparation Example 4-1-2 (8 g, 29.8 mmol) was suspended in phosphorous oxychloride (30 mL) and then stirred under reflux for 16 hours. The resulting mixture was distilled under reduced pressure, then extracted with dichloromethane and purified by column chromatography with the 1:1 mixture of hexane and ethyl acetate to give the title compound (8 g, 93%).

¹H NMR (500 MHz, CDCl₃); δ 7.19 (1H, s), 4.55 (2H, q), 2.97 (2H, t), 1.83 (2H, m), 1.46 (3H, t), 1.03 (3H, t)

### Example 4-1

### 6-Propyl-4-(3-trifluoromethyl-5,6-dihydro-8H-[1,2,4]triazolo[4,3-a]pyrazin-7-yl)thieno[2,3-d]pyrimidine-2-carboxylic acid ethyl ester

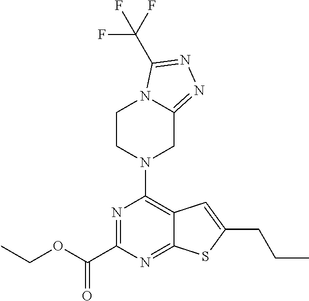

The compound obtained from Preparation Example 4-1-3 (2.04 g, 7.71 mmol) and the compound obtained from Preparation Example 1-1-2 (1.80 g, 7.87 mmol) were diluted with N,N-dimethylformamide (20 mL), then triethylamine (2.34 g, 23.1 mmol) was added thereto at 0° C. and stirred at room temperature for 16 hours. The resulting mixture was distilled under reduced pressure, then diluted with ethyl acetate and washed with water and salt water. Organic layer was dried with anhydrous magnesium sulfate, distilled under reduced pressure and then purified by column chromatography with the 1:1 mixture of hexane and ethyl acetate to give the title compound (2.56 g, 81%).

¹H NMR (500 MHz, CDCl₃); δ 7.11 (1H, s), 5.37 (2H, s), 4.50 (2H, q), 4.43 (4H, s), 2.93 (2H, t), 1.80 (2H, m), 1.45 (3H, t), 1.02 (3H, t)

### Example 4-2

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid

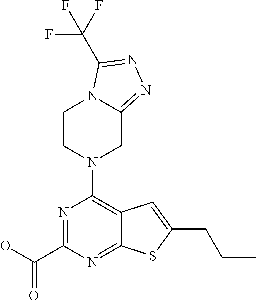

The compound obtained from Example 4-1 (100 mg, 0.23 mmol) was dissolved in tetrahydrofuran (3 mL), methanol (2 mL) and water (1 mL), then lithium hydroxide (14 mg, 0.33 mmol) was added thereto and reacted at room temperature for 4 hours. The resulting mixture was acidified with 1N aqueous hydrochloric acid solution (pH=3), distilled under reduced pressure, then diluted with ethyl acetate and washed with water and salt water. Organic layer was dried with anhydrous magnesium sulfate and distilled under reduced pressure to give the title compound (88.9 mg, 95%).

¹H NMR (500 MHz, CDCl₃); δ 7.19 (1H, s), 5.43 (2H, s), 4.50, 4.44 (4H, two m), 2.95 (2H, t), 1.81 (2H, m), 1.03 (3H, t)

### Example 4-3

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid amide

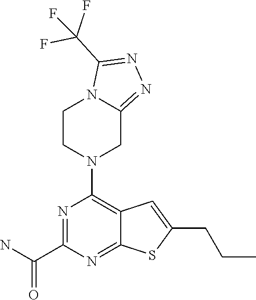

The compound obtained from Example 4-2 (48.7 mg, 0.12 mmol), ammonium chloride (6.95 mg, 0.13 mmol), EDC (27.2 mg, 0.14 mmol) and HOBT (23.9 mg, 0.18 mmol) were dissolved in N,N-dimethylformamide (4 mL), then cooled to 0° C. and diisopropylethylamine (76.3 mg, 0.6 mmol) was added dropwise. The resulting compound was reacted at room temperature for 16 hours, then distilled under reduced pressure, diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (29.9 mg, 62%).

¹H NMR (500 MHz, CDCl₃); δ 7.73 (1H, s), 7.10 (1H, s), 6.40 (1H, s), 5.32 (2H, s), 4.42 (4H, s), 2.91 (2H, t), 1.76 (2H, m), 1.00 (3H, t)

### Preparation Example 4-4-1

### 2-Amino-5-ethylthiophene-3-carboxylic acid methyl ester

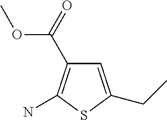

Methyl cyanoacetate (19.8 g, 200 mmol), sulfur (6.4 g, 200 mmol) and triethylamine (10.89 mL, 107.6 mmol) were dissolved in N,N-dimethylformamide (25 mL), heated maintaining the reaction temperature 50° C. and butylaldehyde (14.7 g, 204 mmol) was slowly added dropwise thereto. The resulting mixture was stirred at room temperature for 48 hours, ethyl acetate (100 mL) was added thereto and washed twice with water (100 mL). Organic layer was dried with anhydrous magnesium sulfate, distilled under reduced pressure and then purified by column chromatography with the 4:1 mixture of hexane and ethyl acetate to give the title compound (30.78 g, 83%).

¹H NMR (400 MHz, CDCl₃); δ 6.61 (1H, s), 5.83 (1H, br s), 3.79 (3H, s), 2.62 (3H, q), 1.23 (3H, t)

### Preparation Example 4-4-2

### 6-Ethyl-4-oxo-3,4-dihydrothieno[2,3-d]pyrimidine-2-carboxylic acid methyl ester

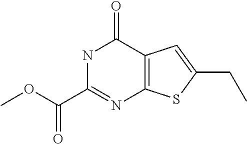

The compound obtained from Preparation Example 4-4-1 (0.43 g, 2.35 mmol) and methyl cyanoformate (0.2 g, 2.35 mmol) were dissolved in dioxane (5 mL) and then cooled to 0° C. 4.0 M hydrochloric acid dioxane solution (5 mL) was slowly added dropwise thereto and the resulting mixture was stirred at room temperature for 16 hours. The resulting mixture was poured to cooled water, then basified with ammonia water and extracted with dichloromethane. Organic layer was dried with anhydrous magnesium sulfate, distilled under reduced pressure and then purified by column chromatography with the 95:5 mixture of dichloromethane and methanol to give the title compound (0.4 g, 71%).

¹H NMR (400 MHz, CDCl₃); δ 10.50 (1H, br s), 7.27 (1H, s), 4.28 (3H, s), 2.94 (2H, q), 1.39 (3H, t)

### Preparation Example 4-4-3

### 4-Chloro-6-ethyl-thieno[2,3-d]pyrimidine-2-carboxylic acid methyl ester

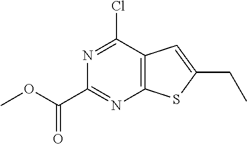

The compound obtained from Preparation Example 4-4-2 (0.4 g, 1.68 mmol) was suspended in phosphorous oxychloride (10 mL) and then stirred under reflux for 16 hours. The resulting mixture was cooled to room temperature, distilled under reduced pressure, extracted with dichloromethane and washed with water. Organic layer was dried with anhydrous magnesium sulfate, distilled under reduced pressure and then purified by column chromatography with the 2:1 mixture of hexane and ethyl acetate to give the title compound (0.36 g, 83%).

¹H NMR (400 MHz, CDCl₃); δ 7.22 (1H, s), 4.10 (3H, s), 3.05 (2H, q), 1.45 (3H, t)

### Preparation Example 4-4-4

### 6-Ethyl-4-(3-trifluoromethyl-5,6-dihydro-8H-[1,2,4]triazolo[4,3-a]pyrazin-7-yl)thieno[2,3-d]pyrimidine-2-carboxylic acid methyl ester

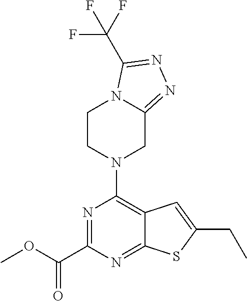

The compound obtained from Preparation Example 4-4-3 (100 mg, 0.39 mmol) and the compound obtained from Preparation Example 1-1-2 (98 mg, 0.42 mmol) were diluted in tetrahydrofuran (4 mL), then diisopropylethylamine (75 mg, 0.58 mmol) was added thereto at 0° C., heated and stirred under reflux for 16 hours. The resulting mixture was distilled under reduced pressure, then diluted with ethyl acetate and washed with water and salt water. Organic layer was dried with anhydrous magnesium sulfate distilled under reduced pressure and then purified by column chromatography with ethyl acetate to give the title compound (158 mg, 98%).

¹H NMR (400 MHz, CDCl₃); δ 7.14 (1H, s), 5.39 (2H, s), 4.45 (4H, s), 4.04 (3H, s), 3.01 (2H, q), 1.42 (3H, t)

### Preparation Example 4-4-5

### {[6-Ethyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid methyl ester

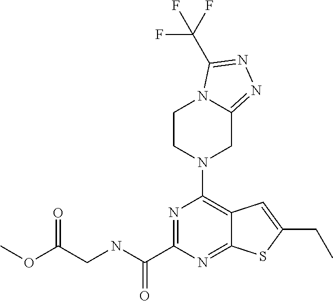

The compound obtained from Preparation Example 4-4-4 (59 mg, 0.14 mmol) was dissolved in the 1:1 mixture of tetrahydrofuran and water, concentrated hydrochloric acid (2 drops) was added thereto, heated and stirred under reflux for 48 hours. The resulting mixture was basified with saturated aqueous sodium hydrogen carbonate solution (pH=5) and then distilled under reduced pressure. Glycine methyl ester; hydrochloric acid salt (54 mg, 0.43 mmol), EDC (96 mg, 0.5 mmol) and HOBT (8 mg, 0.06 mmol) were added to the resulting mixture, tetrahydrofuran (4 mL) was added thereto and then reacted at room temperature for 48 hours. The resulting mixture was distilled under reduced pressure, then diluted with ethyl acetate and washed with water and salt water. Organic layer was dried with anhydrous magnesium sulfate, distilled under reduced pressure and then purified by column chromatography with ethyl acetate to give the title compound (55 mg, 82%).

¹H NMR (400 MHz, DMSO, d₆); δ 9.22 (1H, m), 7.60 (1H, s), 5.37 (2H, s), 4.41 (4H, m), 4.07 (2H, d), 3.67 (3H, s), 3.01 (2H, q), 1.34 (3H, t)

### Example 4-4

### {[6-Ethyl-4-(3-trifluoromethyl-5,6-dihydro-8H-[1,2,4]triazolo[4,3-a]pyrazin-7-yl)-thieno[2,3-d]pyrimidine-2-carbonyl]amino}acetic acid

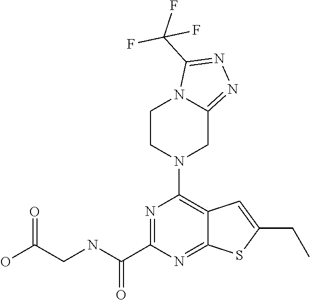

The compound obtained from Preparation Example 4-4-5 (50 mg, 0.106 mmol) was dissolved in tetrahydrofuran, concentrated hydrochloric acid (3 drops) was added thereto, then heated and stirred under reflux for 16 hours. The resulting mixture was basified with basified with saturated aqueous sodium hydrogen carbonate solution (pH=4), then distilled under reduced pressure and then purified by column chromatography with the 80:20 mixture of dichloromethane and methanol to give the title compound (46 mg, 95%).

¹H NMR (400 MHz, DMSO, d₆); δ 8.68 (1H, br s), 7.59 (1H, s), 5.32 (2H, s), 4.43, 4.38 (4H, dd), 3.55 (2H, d), 3.00 (2H, q), 1.34 (3H, t)

### Example 4-5

### 6-Ethyl-4-(3-trifluoromethyl-5,6-dihydro-8H-[1,2,4]triazolo[4,3-a]pyrazin-7-yl)thieno[2,3-d]pyrimidine-2-carboxylic acid (2-hydroxy-ethyl)amide

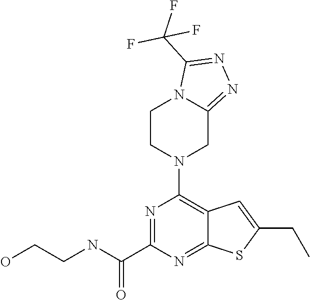

The compound obtained from Example 4-4 (30 mg, 0.066 mmol) and isobutyl chloroformate (10 mg, 0.073 mmol) were dissolved in tetrahydrofuran, N-methyl morpholine (8 mg, 0.079 mmol) was added thereto and stirred at room temperature for 1 hour. Sodium borohydride (3 mg, 0.073 mmol) and water of small quantity was added to the resulting mixture and the mixture was stirred at room temperature for 16 hours. The resulting mixture was distilled under reduced pressure and then purified by column chromatography with the 10:1 mixture of dichloromethane and methanol to give the title compound (4 mg, 14%).

¹H NMR (400 MHz, CDCl₃); δ 8.29 (1H, m), 7.10 (1H, s), 5.33 (2H, s), 4.43 (4H, m), 3.88 (2H, m), 3.69 (2H, m), 3.00 (2H, q), 1.42 (3H, t)

### Example 4-6

### 3-{[6-Propyl-4-(3-trifluoromethyl-5,6-dihydro-8H-[1,2,4]triazolo[4,3-a]pyrazin-7-yl)thieno[2,3-d]pyrimidine-2-carbonyl]amino}propionic acid ethyl ester

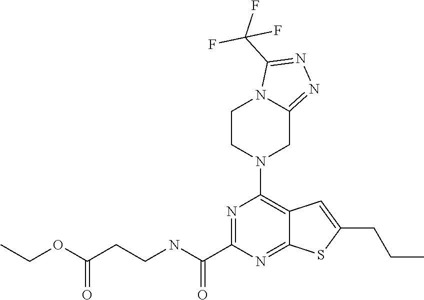

The compound obtained from Example 4-2 (20.5 mg, 0.05 mmol), 3-aminopropionic acid ethyl ester; hydrochloric acid salt (8.4 mg, 0.055 mmol) were dissolved in N,N-dimethylformamide (2 mL) and then HATU (24.6 mg, 0.065 mmol) was added thereto. The resulting mixture was cooled to 0° C., triethylamine (20.1 mg, 0.2 mmol) was added dropwise thereto and stirred at room temperatue for 16 hours. The resulting mixture was distilled under reduced pressure and then purified by column chromatography with the 95:5 mixture of dichloromethane and methanol to give the title compound (22.9 mg, 90%).

¹H NMR (500 MHz, CDCl₃); δ 8.57 (1H, m), 7.09 (1H, s), 5.34 (2H, s), 4.42 (4H, s), 4.16 (2H, q), 3.75 (2H, m), 2.91 (2H, t), 2.65 (2H, m), 1.79 (2H, m), 1.26 (3H, t), 1.01 (3H, t)

### Example 4-7

### 3-{[6-Propyl-4-(3-trifluoromethyl-5,6-dihydro-8H-[1,2,4]triazolo[4,3-a]pyrazin-7-yl)thieno[2,3-d]pyrimidine-2-carbonyl]amino}propionic acid

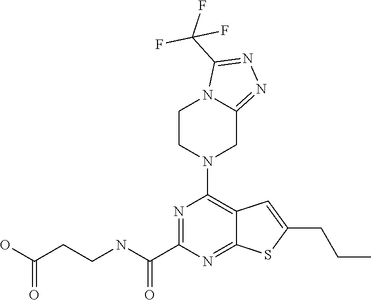

The compound obtained from Example 4-6 (20.7 mg, 0.04 mmol) was dissolved in tetrahydrofuran (3 mL), methanol (2 mL) and water (1 mL), then lithium hydroxide (2.55 mg, 0.06 mmol) was added thereto and reacted at room temperature for 4 hours. The resulting mixture was acidified with 1N aqueous hydrochloric acid solution (pH=3), distilled under reduced pressure, diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (9.8 mg, 50%).

¹H NMR (500 MHz, CDCl₃); δ 8.71 (1H, br s), 7.05 (1H, s), 5.28 (2H, s), 4.42, 4.38 (4H, two m), 3.75 (2H, m), 2.88 (2H, t), 2.70 (2H, m), 1.76 (2H, m), 1.00 (3H, t)

### Example 4-8

### {[6-Propyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid ethyl ester

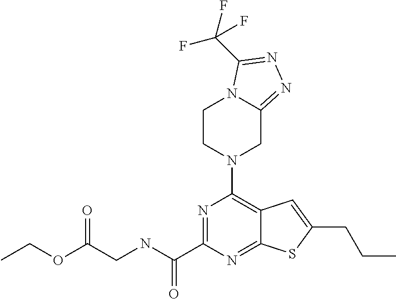

The compound obtained from Example 4-2 (32.5 mg, 0.08 mmol), glycine ethyl ester; hydrochloric acid salt (12.1 mg, 0.087 mmol) were dissolved in N,N-dimethylformamide (3 mL) and then HATU (39.0 mg, 0.1 mmol) was added thereto. The resulting mixture was cooled to 0° C., triethylamine (31.9 mg, 0.32 mmol) was added dropwise thereto and then stirred at room temperature for 16 hours. The resulting mixture was distilled under reduced pressure and then diluted with ethyl acetate and washed with water and salt water. Organic layer was dried with anhydrous magnesium sulfate, distilled under reduced pressure and then purified by column chromatography with the 95:5 mixture of dichloromethane and methanol to give the title compound (19.8 mg, 50%).

¹H NMR (500 MHz, CDCl₃); δ 8.47 (1H, m), 7.07 (1H, s), 5.32 (2H, s), 4.43 (4H, s), 4.26 (4H, m), 2.89 (2H, t), 1.78 (2H, m), 1.31 (3H, t), 1.02 (3H, t)

### Example 4-9

### {[6-Propyl-4-(3-trifluoromethyl-5,6-dihydro-8H-[1,2,4]triazolo[4,3-a]pyrazin-7-yl)thieno[2,3-d]pyrimidine-2-carbonyl]amino}acetic acid

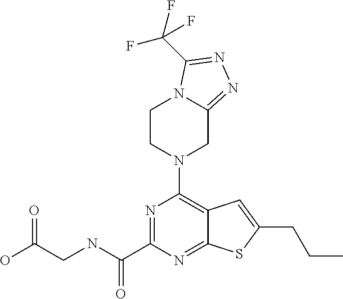

The compound obtained from Example 4-8 (17.8 mg, 0.036 mmol) was dissolved in tetrahydrofuran (3 mL), methanol (2 mL) and water (1 mL), then lithium hydroxide (2.25 mg, 0.054 mmol) was added thereto and reacted at room temperature for 3 hours. The resulting mixture was carefully acidified with 1N aqueous hydrochloric acid solution (pH=4-5), distilled under reduced pressure, diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (8.2 mg, 48%).

¹H NMR (500 MHz, CDCl₃); δ 8.95 (1H, m), 6.89 (1H, br s), 5.20 (2H, s), 4.42 (4H, s), 4.34 (2H, m), 4.18 (2H, m), 2.75 (2H, m), 1.67 (2H, m), 0.97 (3H, m)

### Example 4-10

### 6-Propyl-4-(3-trifluoromethyl-5,6-dihydro-8H-[1,2,4]triazolo[4,3-a]pyrazin-7-yl)thieno[2,3-d]pyrimidine-2-carboxylic acid (2,3-dihydroxy-propyl)amide

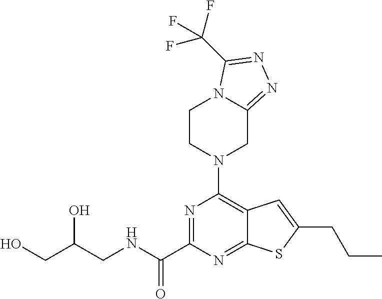

The compound obtained from Example 4-2 (40.5 mg, 0.098 mmol) and 3-amino-1,2-propanediol (8.1 mg, 0.089 mmol) were dissolved in N,N-dimethylformamide (4 mL) and then HATU (43.9 mg, 0.12 mmol) was added thereto. The resulting mixture was cooled to 0° C., triethylamine (36 mg, 0.36 mmol) was added dropwise thereto and then stirred at room temperature for 16 hours. The resulting mixture was distilled under reduced pressure, then diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (13.6 mg, 28%).

¹H NMR (500 MHz, CDCl₃); δ 8.43 (1H, m), 7.08 (1H, s), 5.31 (2H, m), 4.41 (4H, m), 3.93 (1H, m), 3.76˜3.36 (6H, m) 2.91 (2H, t), 1.80 (2H, m), 1.02 (3H, t)

### Example 4-11

### 6-Propyl-4-(3-trifluoromethyl-5,6-dihydro-8H-[1,2,4]triazolo[4,3-a]pyrazin-7-yl)thieno[2,3-d]pyrimidine-2-carboxylic acid (2-cyanoethyl)amide

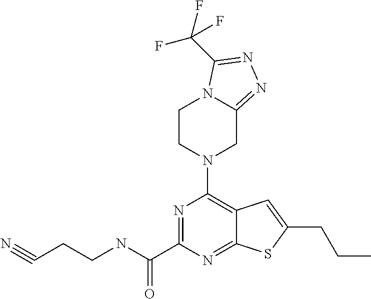

The compound obtained from Example 4-2 (60.2 mg, 0.15 mmol) and 3-amino-propionitrile (11.25 mg, 0.16 mmol) were dissolved in N,N-dimethylformamide (2 mL) and then HATU (72.15 mg, 0.19 mmol) was added thereto. The resulting mixture was cooled to 0° C., triethylamine (59.1 mg, 0.58 mmol) was added dropwise thereto and then stirred at room temperature for 7 hours. The resulting mixture was distilled under reduced pressure, then diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (58.8 mg, 86%).

¹H NMR (500 MHz, CDCl₃); δ 8.37 (1H, m), 7.10 (1H, s), 5.33 (2H, m), 4.42 (4H, m), 3.76 (2H, q), 2.92 (2H, t), 2.77 (2H, t), 1.78 (2H, m), 1.02 (3H, t)

### Example 4-12

### (3-Hydroxy-pyrrolidin-1-yl)-[6-propyl-4-(3-trifluoromethyl-5,6-dihydro-8H-[1,2,4]triazolo[4,3-a]pyrazin-7-yl)thieno[2,3-d]pyrimidin-2-yl]methanon

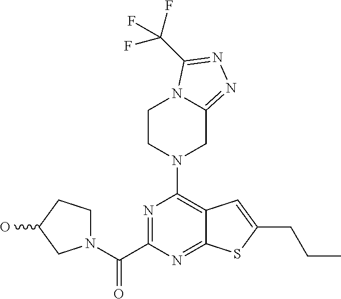

The compound obtained from Example 4-2 (40.0 mg, 0.097 mmol) and pyrrolidine-3-ol (7.68 mg, 0.088 mmol) were dissolved in N,N-dimethylformamide (3 mL) and then HATU (43.6 mg, 0.11 mmol) was added thereto. The resulting mixture was cooled to 0° C., triethylamine (35.7 mg, 0.35 mmol) was added dropwise thererto and then stirred at room temperature for 6 hours. The resulting mixture was distilled under reduced pressure, then diluted with ethyl acetate and washed with water and salt water. Organic layer was dried with anhydrous magnesium sulfate, distilled under reduced pressure and then 10:1 purified by column chromatography with the 10:1 mixture of dichloromethane and methanol to give the title compound (11.8 mg, 25%).

¹H NMR (500 MHz, CDCl₃); δ 7.07 (1H, s), 5.32 (2H, d), 4.58, 4.50 (1H, two m), 4.38 (4H, m), 3.83˜3.59 (4H, m), 2.90 (2H, t), 2.52 (1H, br s), 2.01 (2H, m), 1.77 (2H, m), 1.02 (3H, t)

### Example 4-13

### (4-Methyl-piperazin-1-yl)-[6-propyl-4-(3-trifluoromethyl-5,6-dihydro-8H-[1,2,4]triazolo[4,3-a]pyrazin-7-yl)thieno[2,3-d]pyrimidin-2-yl]methanone

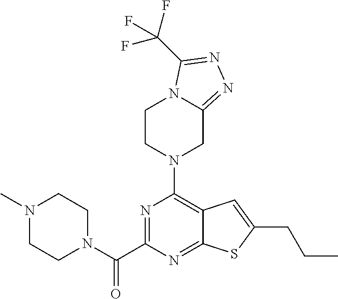

The compound obtained from Example 4-2 (30.5 mg, 0.074 mmol) and 1-methylpiperazine (6.7 mg, 0.067 mmol) were dissolved in N,N-dimethylformamide (2 mL) and then HATU (33.06 mg, 0.087 mmol) was added thereto. The resulting mixture was cooled to 0° C., triethylamine (27.1 mg, 0.27 mmol) was added dropwise thereto and then stirred at room temperature for 6 hours. The resulting mixture was distilled under reduced pressure and then diluted with ethyl acetate and washed with water and salt water. Organic layer was dried with anhydrous magnesium sulfate, distilled under reduced pressure and then purified by column chromatography with the 10:1 mixture of dichloromethane and methanol to give the title compound (6.3 mg, 17%).

¹H NMR (500 MHz, CDCl₃); δ 7.06 (1H, s), 5.33 (2H, s), 4.37 (4H, s), 3.86 (2H, m), 3.40 (2H, m), 2.90 (2H, m), 2.53 (2H, m), 2.38 (2H, m), 2.32 (3H, s), 1.80 (2H, m), 1.02 (3H, t)

### Example 4-14

### 1-[6-Propyl-4-(3-trifluoromethyl-5,6-dihydro-8H-[1,2,4]triazolo[4,3-a]pyrazin-7-yl)thieno[2,3-d]pyrimidine-2-carbonyl]piperidine-3-carboxylic acid ethyl ester

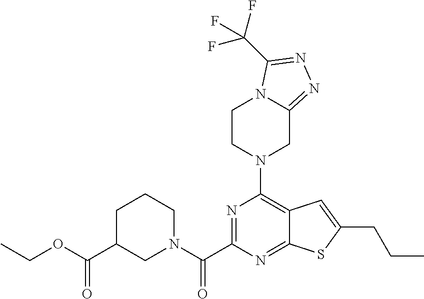

The compound obtained from Example 4-2 (30.4 mg, 0.074 mmol) and piperidine-3-carboxylic acid ethyl ester (10.54 mg, 0.067 mmol) were dissolved in N,N-dimethylformamide (3 mL) and then HATU (33.14 mg, 0.087 mmol) was added thereto. The resulting mixture was cooled to 0° C., triethylamine (27.1 mg, 0.27 mmol) was added dropwise thereto and then stirred at room temperature for 16 hours. The resulting mixture was distilled under reduced pressure, then diluted with ethyl acetate and washed with water and salt water. Organic layer was dried with anhydrous magnesium sulfate and distilled under reduced pressure and then purified by column chromatography with the 98:2 mixture of dichloromethane and methanol to give the title compound (30.6 mg, 75%).

¹H NMR (500 MHz, CDCl₃); δ 7.06 (1H, s), 5.31 (2H, m), 4.78, 4.50 (1H, two m), 4.35 (4H, m), 4.16 (1H, m), 4.02 (1H, m), 3.74, 3.51 (1H, two m), 3.21, 2.93 (2H, two m), 2.88 (2H, m), 2.61 (1H, m), 2.15 (1H, m), 1.85˜1.55 (5H, m), 1.26, 1.15 (3H, two t), 1.00 (3H, t)

### Example 4-15

### 1-[6-Propyl-4-(3-trifluoromethyl-5,6-dihydro-8H-[1,2,4]triazolo[4,3-a]pyrazin-7-yl)thieno[2,3-d]pyrimidine-2-carbonyl]piperidine-3-carboxylic acid

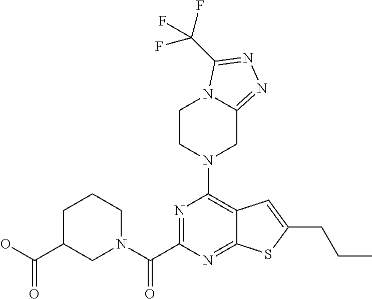

The compound obtained from Example 4-14 (25.6 mg, 0.046 mmol) was dissolved in tetrahydrofuran (3 mL), methanol (2 mL) and water (1 mL), lithium hydroxide (2.92 mg, 0.069 mmol) was added thereto and reacted at room temperature for 4 hours. The resulting mixture was acidified with 1N aqueous hydrochloric acid solution (pH=3), distilled under reduced pressure and then washed with ethyl acetate and salt water. Organic layer was dried with anhydrous magnesium sulfate and distilled under reduced pressure and then purified by column chromatography with the 10:1 mixture of dichloromethane and methanol to give the title compound (13.8 mg, 57%).

¹H NMR (500 MHz, CD₃OD); δ 7.41 (1H, s), 5.34 (2H, d), 4.78, 4.50 (1H, two m), 4.42 (4H, m), 3.73, 3.51 (1H, two m), 3.21, 2.93 (2H, two m), 2.96 (2H, m), 2.55 (1H, m), 2.16 (1H, m), 1.87-1.57 (5H, m), 1.02 (3H, t)

### Example 4-16

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid phenylamide

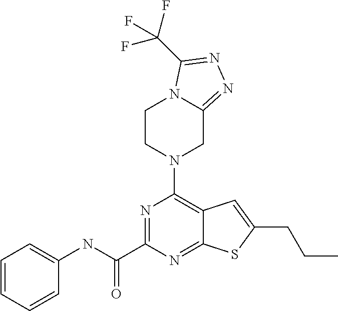

The compound obtained from Example 4-2 (24 mg, 0.06 mmol) and aniline (4.9 mg, 0.053 mmol) were dissolved in N,N-dimethylformamide (2 mL) and then HATU (26.2 mg, 0.069 mmol) was added thereto. The resulting mixture was cooled to 0° C., triethylamine (21.3 mg, 0.21 mmol) was added dropwise thereto and then stirred at room temperature for 16 hours. The resulting mixture was distilled under reduced pressure, then diluted with ethyl acetate and washed with water and salt water. Organic layer was dried with anhydrous magnesium sulfate and distilled under reduced pressure and then purified by column chromatography with the 95:5 mixture of dichloromethane and methanol to give the title compound (20.5 mg, 72%).

¹H NMR (500 MHz, CDCl₃); δ 9.82 (1H, s), 7.81 (2H, d), 7.41 (2H, t), 7.17 (1H, m), 7.14 s), 5.39 (2H, s), 4.50, 4.46 (4H, two m), 2.95 (2H, t), 1.81 (2H, m), 1.04 (3H, t)

### Example 4-17

### 6-Propyl-4-(3-trifluoromethyl-5,6-dihydro-8H-[1,2,4]triazolo[4,3-a]pyrazin-7-yl)thieno[2,3-d]pyrimidine-2-carboxylic acid benzylamide

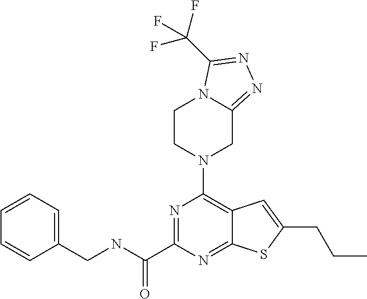

The compound obtained from Example 4-2 (25.1 mg, 0.061 mmol) and benzylamine (5.9 mg, 0.055 mmol) were dissolved in N,N-dimethylformamide (2 mL) and then HATU (27.3 mg, 0.072 mmol) was added thereto. The resulting mixture was cooled to 0° C., triethylamine (22.3 mg, 0.22 mmol) was added dropwise thereto and then stirred at room temperature for 16 hours. The resulting mixture was distilled under reduced pressure, then diluted with ethyl acetate and washed with water and salt water. Organic layer was dried with anhydrous magnesium sulfate, distilled under reduced pressure and then purified by column chromatography with the 95:5 mixture of dichloromethane and methanol to give the title compound (23.1 mg, 75%).

¹H NMR (500 MHz, CDCl₃); δ 8.18 (1H, m), 7.36 (4H, m), 7.29 (1H, m), 7.09 (1H, s), 5.34 (2H, s), 4.70 (2H, d), 4.42 (4H, m), 2.92 (2H, t), 1.79 (2H, m), 1.02 (3H, t)

### Example 4-18

### [6-Ethyl-4-(3-trifluoromethyl-5,6-dihydro-8H-[1,2,4]triazolo[4,3-a]pyrazin-7-yl)thieno[2,3-d]pyrimidin-2-yl]methanol

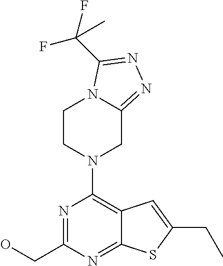

The compound obtained from Example 4-4-4 (97 mg, 0.235 mmol) was dissolved in tetrahydrofuran (10 mL), lithium aluminum hydride (10 mg, 0.258 mmol) was slowly added thereto at 0° C. and then stirred for 1 hours. Water (10 uL), 15% aqueous sodium hydroxide solution (10 uL), water (30 uL) were added to the reaction mixture at 0° C. and stirred for 30 minutes at room temperature. The reaction mixture was dried with anhydrous magnesium sulfate, then distilled under reduced pressure and purified by column chromatography with ethyl acetate to give the title compound (20 mg, 22%).

¹H NMR (400 MHz, CDCl₃); δ 7.03 (1H, s), 5.33 (2H, s), 4.74 (2H, s), 4.37 (2H, m), 4.33 (2H, m), 2.95 (2H, t), 1.40 (3H, t)

### Example 4-19

### [6-Propyl-4-(3-trifluoromethyl-5,6-dihydro-8H-[1,2,4]triazolo[4,3-a]pyrazin-7-yl)-thieno[2,3-d]pyrimidin-2-yl]methanol

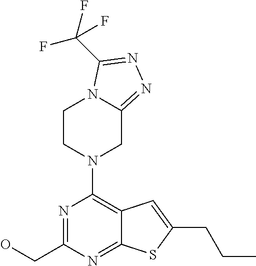

The compound obtained from Example 4-1 (370 mg, 0.84 mmol) was reacted according to the similar method to Example 4-18 to give the title compound (128 mg, 38%).

¹H NMR (500 MHz, CDCl₃); δ 7.02 (1H, s), 5.32 (2H, s), 4.72 (2H, d), 4.37 (2H, m), 4.33 (2H, m), 3.48 (1H, t), 2.89 (2H, t), 1.79 (2H, m), 1.02 (3H, t)

### Preparation Example 4-20-1

### 7-(2-Chloromethyl-6-ethyl-thieno[2,3-d]pyrimidin-4-yl)-3-trifluoromethyl-5,6,7,8-tetrahydro-[1,2,4]triazolo[4,3-a]pyrazine

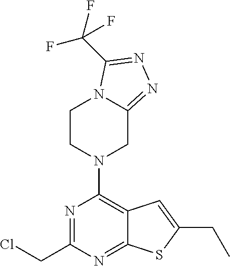

The compound obtained from Example 4-18 (48 mg, 0.125 mmol) was dissolved in dichloromethane (5 mL), then p-toluenesulfonyl chloride (59 mg, 0.150 mmol) and triethylamine (35 uL, 0.250 mmol) were added thereto and stirred for 48 hours. The resulting mixture was distilled under reduced pressure and then purified by column chromatography with the 1:1 mixture of ethyl acetate and hexane to give the title compound (40 mg, 77%).

¹H NMR (500 MHz, CDCl₃); δ 7.04 (1H, s), 5.33 (2H, s), 4.64 (2H, s), 4.40 (2H, m), 4.35 (2H, m), 2.95 (2H, t), 1.39 (3H, t)

### Example 4-20

### (R)-1-[6-Ethyl-4-(3-trifluoromethyl-5,6-dihydro-8H-[1,2,4]triazolo[4,3-a]pyrazin-7-yl)thieno[2,3-d]pyrimidin-2-ylmethyl]pyrrolidin-3-ol

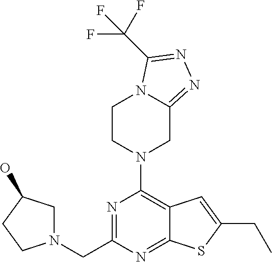

The compound obtained from Preparation Example 4-20-1 (40 mg, 0.099 mmol), (R)-pyrrolidine-3-ol; hydrochloric acid salt (18.41 mg, 0.149 mmol) and cesium carbonate (97 mg, 0.298 mmol) were dissolved in N,N-dimethylformamide (5 mL) and stirred at 60° C. for 4 hours. The resulting mixture was distilled under reduced pressure and purified by column chromatography with the 1:10 mixture of dichloromethane and methanol to give the title compound (10 mg, 22%).

¹H NMR (400 MHz, CDCl₃); δ 7.01 (1H, s), 5.30 (2H, s), 4.37 (5H, m), 3.92 (2H, dd), 3.15 (1H, m), 2.96 (4H, m), 2.80 (1H, m), 2.63 (1H, m), 2.17 (1H, m), 2.02 (1H, m), 1.85 (1H, m), 1.39 (3H, t)

### Preparation Example 4-21-1

### 7-(2-Chloromethyl-6-propyl-thieno[2,3-d]pyrimidin-4-yl)-3-trifluoromethyl-5,6,7,8-tetrahydro-[1,2,4]triazolo[4,3-a]pyrazine

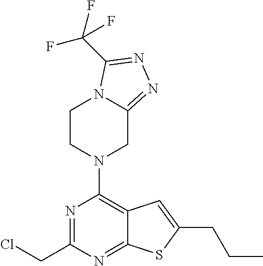

The compound obtained from Example 4-19 (128 mg, 0.321 mmol) and p-toluenesulfonyl chloride (74 mg, 0.386 mmol) were reacted according to the similar method to Preparation Example 4-20-1 to give the title compound (40 mg, 30%).

¹H NMR (500 MHz, CDCl₃); δ 7.04 (1H, s), 5.33 (2H, s), 4.64 (2H, s), 4.40 (2′, m), 4.35 (2H, m), 2.89 (2H, t), 1.78 (2H, m), 1.39 (3H, t)

### Example 4-21

### (R)-1-[6-Propyl-4-(3-trifluoromethyl-5,6-dihydro-8H-[1,2,4]triazolo[4,3-a]pyrazin-7-yl)thieno[2,3-d]pyrimidin-2-ylmethyl]pyrrolidin-3-ol

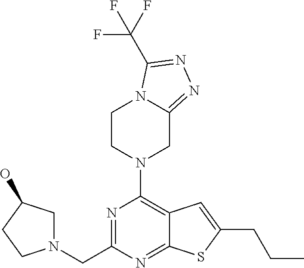

The compound obtained from Preparation Example 4-21-1 (40 mg, 0.096 mmol), (R)-pyrrolidine-3-ol; hydrochloric acid salt (63 mg, 0.192 mmol) were reacted according to the similar method to Example 4-20 to give the title compound (30 mg, 67%).

¹H NMR (400 MHz, CDCl₃); δ 7.01 (1H, s), 5.30 (2H, s), 4.36 (5H, m), 3.91 (2H, dd), 3.10 (1H, m), 2.84 (4H, m), 2.63 (1H, m), 2.21 (1H, m), 1.81 (3H, m), 1.02 (3H, t)

### Preparation Example 4-22-1

### Benzoic acid 4-oxo-6-propyl-3,4-dihydro-thieno[2,3-d]pyrimidin-2-ylmethyl ester

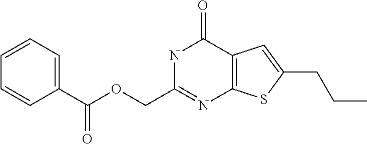

The compound obtained from Preparation Example 4-1-1 (5.0 g, 25.09 mmol) and benzoic acid cyanomethyl ester (4.45 g, 27.60 mmol) were diluted in dioxane (20 mL), 4.0 M hydrochloric acid dioxane solution (15 mL) was added thereto and stirred at room temperature for 16 hours. Thus obtained solid was filtrated, washed with water and hexane and then dried to give the title compound (6.0 g, 73%).

¹H NMR (400 MHz, CDCl₃); δ10.82 (1H, br s), 8.11 (2H, m), 7.61 (1H, m), 7.45 (2H, m), 6.97 (1H, s), 5.26 (2H, s), 2.80 (2H, t), 1.74 (2H, m), 0.99 (3H, t)

### Preparation Example 4-22-2

### Benzoic acid 4-chloro-6-propyl-thieno[2,3-d]pyrimidin-2-ylmethyl ester

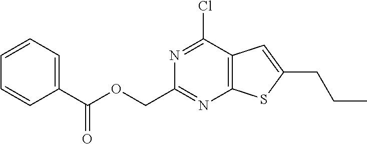

The compound obtained from Preparation Example 4-22-1 (3.0 g, 9.14 mmol) was supended in phosphorous oxychloride (30 mL) and stirred under reflux for 2 hours. The resulting mixture was distilled under reduced pressure and purified by column chromatography with dichloromethane to give the title compound (2.7 g, 85%).

¹H NMR (400 MHz, CDCl₃); δ8.13 (2H, m), 7.57 (1H, m), 7.45 (2H, m), 7.08 (1H, s), 5.58 (2H, s), 2.92 (2H, t), 1.79 (2H, m), 1.03 (3H, t)

### Example 4-22

### Benzoic Acid

### 6-propyl-4-(3-trifluoromethyl-5,6-dihydro-8H-[1,2,4]triazolo[4,3-a]pyrazin-7-yl)thieno[2,3-d]pyrimidin-2-ylmethyl ester

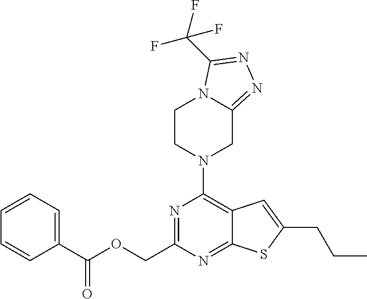

The compound obtained from Preparation Example 4-22-2 (800 mg, 2.44 mmol) and the compound obtained from Preparation Example 1-1-2 (613 mg, 2.68 mmol) were diluted in N,N-dimethylformamide (10 mL), then diisopropylethylamine (1.27 mL, 7.31 mmol) was added thereto and the resulting mixture was stirred at 70° C. for 4 hours. Ethyl acetate (30 mL) was added thereto and the resulting mixture was washed twice with water (30 mL) and then organic layer was dried with anhydrous magnesium sulfate. Organic layer was distilled under reduced pressure and purified by column chromatography with the 1:4 mixture of ethyl acetate and dichloromethane to give the title compound (880 mg, 72%).

¹H NMR (400 MHz, CDCl₃); δ 8.15 (2H, m), 7.62 (1H, m), 7.50 (2H, m), 7.00 (1H, s), 5.50 (2H, s), 5.27 (2H, s), 4.12 (2H, m), 4.02 (2H, m), 2.89 (2H, t), 1.77 (2H, m), 1.02 (3H, t)

### Example 4-23

### 7-(2-Phenoxymethyl-6-propyl-thieno[2,3-d]pyrimidin-4-yl)-3-trifluoromethyl-5,6,7,8-tetrahydro-[1,2,4]triazolo[4,3-a]pyrazine

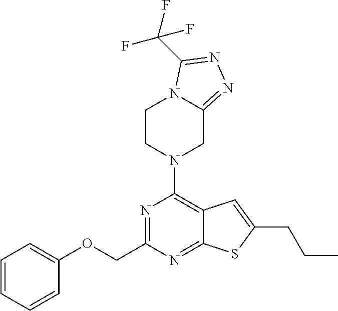

The compound obtained from Example 4-19 (50 mg, 0.126 mmol) was dissolved in tetrahydrofuran (5 mL), and phenol (15 mg, 0.163 mmol), triphenylphosphin (49 mg, 0.188 mmol) and DIAD (0.037 mL, 0.188 mmol) were added successively thereto at 0° C. and then stirred at room temperature for 16 hours. The resulting mixture was distilled under reduced pressure and purified by column chromatography with the 1:1 mixture of ethyl acetate and hexane to give the title compound (25 mg, 42%).

¹H NMR (500 MHz, CDCl₃); δ7.26 (2H, m), 7.01˜6.94 (4H, m), 5.28 (2H, s), 5.24 (2H, s), 4.18 (2H, m), 4.08 (2H, m), 2.88 (2H, t), 1.76 (2H, m), 1.01 (3H, t)

### Example 4-24

### 2-[6-Propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid methyl ester

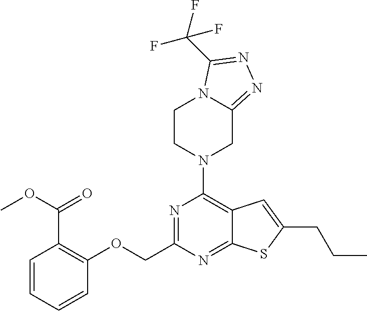

The compound obtained from Example 4-19 (50 mg, 0.126 mmol) and 2-hydroxy-benzoic acid methyl ester (25 mg, 0.163 mmol) were reacted according to the similar method to Example 4-23 to give the title compound (30 mg, 45%).

¹H NMR (400 MHz, CDCl₃); δ 7.77 (1H, m), 7.35 (1H, m), 7.01˜6.96 (4H, m), 5.37 (2H, s), 5.29 (2H, s), 4.16 (2H, m), 4.02 (2H, m), 3.92 (3H, s), 2.89 (2H, t), 1.77 (2H, m), 1.02 (3H, t)

### Example 4-25

### 2-[6-Propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid

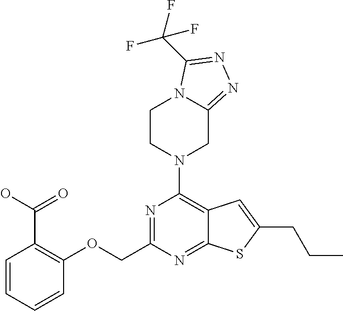

The compound obtained from Example 4-24 (30 mg, 0.056 mmol) was dissolved in the 5:3:1 mixture of tetrahydrofuran, water and methanol, then lithium hydroxide (4.73 mg, 0.112 mmol) was added thereto and the resulting mixture was stirred at room temperature for 6 hours. The resulting mixture was distilled under reduced pressure and then purified by column chromatography with the mixture of ethyl acetate and hexane to give the title compound (4.5 mg, 15%).

¹H NMR (400 MHz, CDCl₃); δ 8.19 (1H, m), 7.55 (1H, m), 7.17 (2H, m), 7.07 (1H, s), 5.42 (2H, s), 5.37 (2H, s), 4.37 (4H, m), 2.90 (2H, t), 1.79 (2H, m), 1.03 (3H, t)

### Preparation Example 4-26-1

### 3-[6-Propyl-4-(3-trifluoromethyl-5,6-dihydro-8H-[1,2,4]triazolo[4,3-a]pyrazin-7-yl)-thieno[2,3-d]pyrimidin-2-ylmethoxy]-benzoic acid methyl ester

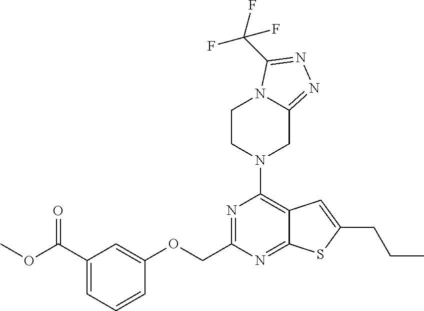

The compound obtained from Example 4-19 (50 mg, 0.126 mmol) and 3-hydroxy-benzoid acid methyl ester (25 mg, 0.163 mmol) were reacted according to the similar method to Example 4-23 to give the title compound (10 mg, 15%).

¹NMR (400 MHz, CDCl₃); δ 7.77 (1H, m), 7.62 (1H, m), 7.33 (1H, m), 7.19 (1H, m), 7.03 (1H, s), 5.30 (2H, s), 5.29 (2H, s), 4.23 (2H, m), 4.16 (2H, m), 3.89 (3H, s), 2.89 (2H, t), 1.78 (2H, m), 1.02 (3H, t)

### Example 4-26

### 3-[6-Propyl-4-(3-trifluoromethyl-5,6-dihydro-8H-[1,2,4]triazolo[4,3-a]pyrazin-7-yl)thieno[2,3-d]pyrimidin-2-ylmethoxy]benzoic acid

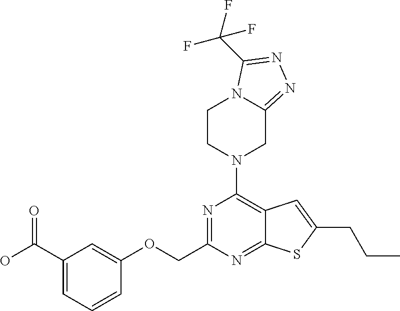

The compound obtained from Preparation Example 4-26-1 (10 mg, 0.019 mmol) was reacted according to the similar method to Example 4-25 to give the title compound (4.5 mg, 46%).

¹H NMR (400 MHz, CD₃OD); δ 7.69 (1H, s), 7.60 (1H, d), 7.35 (2H, m), 7.22 (1H, m), 5.30 (2H, s), 5.26 (2H, s), 4.32 (4H, t), 2.95 (2H, t), 1.85 (2H, m), 1.03 (3H, t)

### Preparation Example 4-27-1

### N-Hydroxy-propionamidine

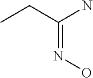

Hydroxylamine; hydrochloric acid salt (1.32 g, 19.1 mmol) was dissolved in methanol (20 mL), then sodium hydrogen carbonate (1.6 g, 19.1 mmol) was added thereto and stirred at room temperature for 20 minutes. Propionitrile (1 g, 18.2 mmol) was added thereto, heated, stirred under reflux for 16 hours and then cooled to room temperature. Thus obtained salt was removed by filtration and the filtrate was distilled under reduced pressure and then purified by column chromatography with the 10:1 mixture of dichloromethane and methanol to give the title compound (1.12 g, 70%).

¹H NMR (500 MHz, CDCl₃); δ 4.49 (2H, br s), 2.17 (2H, m), 1.14 (3H, t)

### Example 4-27

### 7-[2-(3-Ethyl-[1,2,4]oxadiazol-5-yl)-6-propyl-thieno[2,3-d]pyrimidin-4-yl]-3-trifluoromethyl-5,6,7,8-tetrahydro-[1,2,4]triazolo[4,3-a]pyrazine

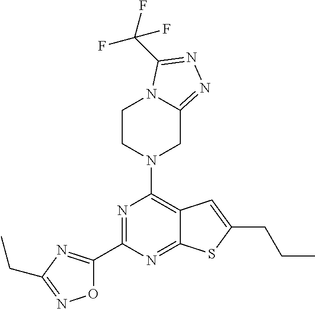

The compound (38.9 mg, 0.094 mmol) obtained from Example 4-2 was dissolved in dichloromethane 2 mL and oxalyl chloride (35.9 mg, 0.28 mmol) was added thereto. After the reaction mixture was cooled to 0° C., catalytic amounts of N,N-dimethylformamide were added thereto and stirred for 2 hours at room temperature. After distillation of the reaction mixture under reduced pressure and drying, the compound (9.14 mg, 0.10 mmol) obtained from Preparation Example 4-27-1 and pyridine 3 mL were added thereto, suspended and heated to 120° C. After stirring for 4 hours at 120° C., cooling to room temperature and distillation under reduced pressure, the title compound (7.5 mg, 17%) was obtained by column-chromatography using 95:5 mixture of dichloromethane and methanol.

¹H NMR (500M z, CDCl₃); δ 7.16 (1H, s), 5.42 (2H, s), 4.48 (4H, m), 2.95 (2H, t), 2.91 (2H, q), 1.82 (2H, m), 1.42 (3H, t), 1.04 (3H, t)

### Example 4-28

### 7-[2-(3-Phenyl-[1,2,4]oxadiazol-5-yl)-6-propyl-thieno[2,3-d]pyrimidin-4-yl)-3-trifluoromethyl-5,6,7,8-tetrahydro-[1,2,4]triazolo[4,3-a]pyrazine

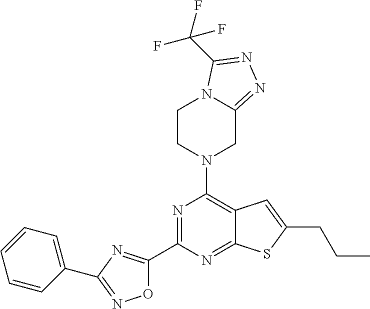

The compound (36.4 mg, 0.088 mmol) obtained from Example 4-2 was dissolved in dichloromethane 2 mL and oxalyl chloride (33.6 mg, 0.26 mmol) was added thereto. After the reaction mixture was cooled to 0° C., catalytic amounts of N,N-dimethylformamide were added thereto and stirred for 3 hours at room temperature. After distillation of the reaction mixture under reduced pressure and drying, N-hydroxybenzamidine (15.6 mg, 0.11 mmol) and pyridine 3 mL were added thereto, suspended and heated to 120° C. After stirring for 4 hours at 120° C., cooling to room temperature and distillation under reduced pressure, the title compound (8.6 mg, 19%) was obtained by column-chromatography using 10:1 mixture of dichloromethane and methanol.

¹H NMR (500 MHz, CDCl₃); δ 8.24 (2H, d), 7.51 (3H, m), 7.18 (1H, s), 5.45 (2H, s), 4.50 (4H, m), 2.96 (2H, t), 1.82 (2H, m), 1.05 (3H, t)

### Preparation Example 4-29-1

### N-Hydroxy-3-methoxypropionamidine

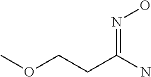

Hydrochloric acid salt of hydroxylamine (171.5 mg, 2.47 mmol) was dissolved in methanol 4 mL, sodium hydrogen carbonate (207.3 mg, 2.47 mmol) was added thereto and then, stirred for 20 minutes at room temperature. 3-methoxypropionitrile (200 mg, 2.35 mmol) was added thereto, heated, and stirred for 16 hours with reflux and then, cooled to room temperature. Formed salt was filtrated and removed. After distillation of the filtrate under reduced pressure, the title compound (164.3 mg, 59%) was obtained by column-chromatography using 10:1 mixture of dichloromethane and methanol.

Mass: M+H 119

### Example 4-29

### 7-{2-[3-(2-Methoxy-ethyl)-[1,2,4]oxadiazol-5-yl]-6-propyl-thieno[2,3-d]pyrimidin-4-yl}-3-trifluoromethyl-5,6,7,8-tetrahydro-[1,2,4]triazolo[4,3-a]pyrazine

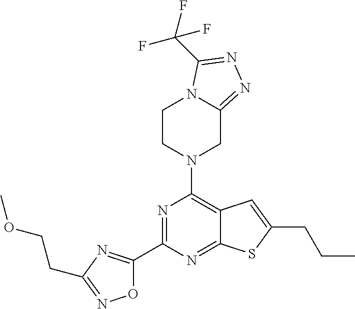

The compound (43.0 mg, 0.10 mmol) obtained from Example 4-2 was dissolved in dichloromethane 3 mL and oxalyl chloride (39.7 mg, 0.31 mmol) was added thereto. After the reaction mixture was cooled to 0° C., catalytic amounts of N,N-dimethylformamide were added thereto and stirred for 4 hours at room temperature. After distillation of the reaction mixture under reduced pressure and drying, the compound (16.0 mg, 0.14 mmol) obtained from Preparation Example 4-29-1 and pyridine 3 mL were added thereto, suspended and heated to 120° C. After stirring for 16 hours at 120° C., cooling to room temperature and distillation under reduced pressure, the title compound (8.1 mg, 16%) was obtained by column-chromatography using 95:5 mixture of dichloromethane and methanol.

¹H NMR (500 MHz, CDCl₃); δ 7.16 (1H, s), 5.40 (2H, s), 4.47 (4H, m), 3.88 (2H, t), 3.38 (3H, s), 3.15 (2H, t), 2.95 (2H, t), 1.81 (2H, m), 1.05 (3H, t)

### Example 4-30

### 2-{5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,2,4]oxadiazol-3-yl}ethanol

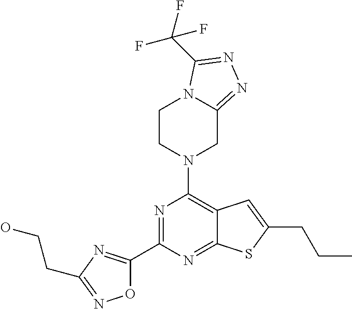

The compound (47.8 mg, 0.097 mmol) obtained from Example 4-29 was dissolved in distilled dichloromethane 4 mL, cooled to −78° C. under N₂ gas and then 1.0 M boron tribromide dichloromethane solution (0.18 mL, 0.18 mmol) was added thereto. The reaction mixture was slowly heated to room temperature, stirred and then cooled to 0° C., again. The reaction was terminated with saturated ammonium chloride aqueous solution. The reaction mixture was diluted with ethyl acetate and washed with brine. After the organic layer was dried with anhydrous magnesium sulfate and distilled under reduced pressure, the title compound (29.1 mg, 63%) was obtained by column-chromatography using 10:1 mixture of dichloromethane and methanol.

¹H NMR (500 MHz, CDCl₃); δ 7.17 (1H, s), 5.43 (2H, s), 4.48 (4H, m), 4.13 (2H, m), 3.15 (2H, t), 2.96 (2H, t), 2.21 (1H, m), 1.82 (2H, m), 1.04 (3H, t)

### Preparation Example 4-31-1

### N-Hydroxy-3,3-dimethoxypropionamidine

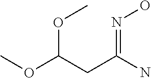

Hydrochloric acid salt of hydroxylamine (126.8 mg, 1.82 mmol) was dissolved in methanol 4 mL, sodium hydrogen carbonate (153.2 mg, 1.82 mmol) was added thereto and then, stirred for 20 minutes at room temperature. 3,3-dimethoxypropionitrile (200 mg, 1.74 mmol) was added thereto, heated, and stirred for 16 hours with reflux and then, cooled to room temperature. Formed salt was filtrated and removed. After distillation of the filtrate under reduced pressure, the title compound (224.1 mg, 87%) was obtained by column-chromatography using 95:5 mixture of dichloromethane and methanol.

Mass: M+H 149

### Example 4-31

### 7-{2-[3-(2,2-Dimethoxy-ethyl)-[1,2,4]oxadiazol-5-yl]-6-propyl-thieno[2,3-d]pyrimidin-4-yl}-3-trifluoromethyl-5,6,7,8-tetrahydro-[1,2,4]triazolo[4,3-a]pyrazine

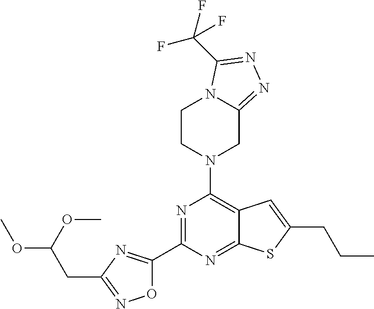

The compound (72.0 mg, 0.17 mmol) obtained from Example 4-2 was dissolved in dichloromethane 4 mL and oxalyl chloride (66.5 mg, 0.52 mmol) was added thereto. After the reaction mixture was cooled to 0° C., catalytic amounts of N,N-dimethylformamide were added thereto and stirred for 4 hours at room temperature. After distillation of the reaction mixture under reduced pressure and drying, the compound (33.6 mg, 0.23 mmol) obtained from Preparation Example 4-31-1 and pyridine 4 mL were added thereto, suspended and heated to 120° C. After stirring for 16 hours at 120° C., cooling to room temperature and distillation under reduced pressure, the title compound (4.2 mg, 5%) was obtained by column-chromatography using 1:1 mixture of hexane and ethyl acetate.

¹H NMR (500 MHz, CDCl₃); δ 7.16 (1H, s), 5.43 (2H, s), 5.05 (1H, t), 4.47 (4H, m), 3.39 (6H, s), 3.21 (2H, d), 2.95 (2H, t), 1.82 (2H, m), 1.04 (3H, t)

### Preparation Example 4-32-1

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid N′-acetyl-hydrazide

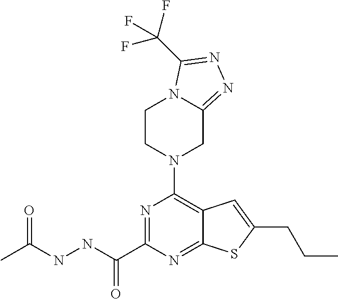

The compound (32.0 mg, 0.078 mmol) obtained from Example 4-2 and acetic acid hydrazide (6.32 mg, 0.085 mmol) were dissolved in N,N-dimethylformamide 2 mL and HATU (38.4 mg, 0.01 mmol) was added thereto. After the reaction mixture was cooled to 0° C., triethylamine (31.4 mg, 0.31 mmol) was added dropwise thereto and stirred for 5 hours at room temperature. After distillation of the reaction mixture under reduced pressure, the title compound (31.3 mg, 86%) was obtained by column-chromatography using 10:1 mixture of dichloromethane and methanol.

¹H NMR (500 MHz, CDCl₃); δ 7.00 (1H, s), 5.35 (2H, s), 4.57 (2H, m), 4.41 (2H, m), 2.76 (2H, t), 2.13 (3H, s), 1.82 (2H, m), 0.95 (3H, t)

### Example 4-32

### 7-[2-(5-Methyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine

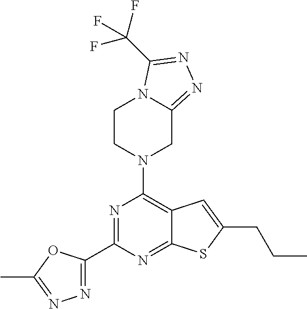

The compound (31.3 mg, 0.067 mmol) obtained from Preparation Example 4-32-1 was dissolved in dichloromethane 2 mL, 2-chloro-1,3-dimethylimidazolinium chloride (11.3 mg, 0.067 mmol) and triethylamine (13.5 mg, 0.13 mmol) were added thereto and then, stirred for 16 hours at room temperature. Since the reaction was not completed, 2-chloro-1,3-dimethylimidazolinium chloride (11.3 mg, 0.067 mmol) and triethylamine (13.5 mg, 0.13 mmol) were additionally added thereto, heated to 60° C. and stirred for 24 hours. After the reaction mixture was cooled to room temperature and distilled under reduced pressure, the title compound (2.4 mg, 8%) was obtained by column-chromatography using 95:5 mixture of dichloromethane and methanol.

¹H NMR (5031-7, CDCl₃); δ 7.16 (1H, s), 5.42 (2H, s), 4.48, 4.45 (4, two m), 2.94 (2H, t), 2.69 (3H, s), 1.81 (2H, m), 1.04 (3H, t)

### Preparation Example 4-33-1

### Benzoic Acid

### N′-[6-propyl-4-(3-trifluoromethyl-5,6-dihydro-8H-[1,2,4]triazolo[4,3-a]pyrazin-7-yl)-thieno[2,3-d]pyrimidine-2-carbonyl]hydrazide

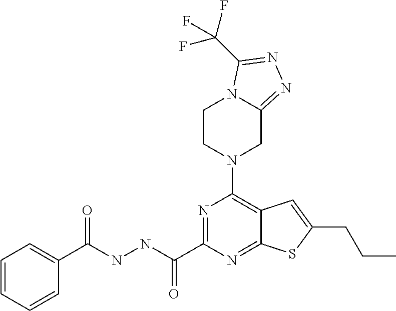

The compound (42.7 mg, 0.10 mmol) obtained from Example 4-2, benzoylhydrazine (16.9 mg, 0.12 mmol), EDC (25.8 mg, 0.13 mmol) and HOBT (18.2 mg, 0.13 mmol) were cooled to 0° C. and dissolved in N,N-dimethylformamide 4 mL. Triethylamine (41.9 mg, 0.41 mmol) was added dropwise thereto. After the reaction for 16 hours at room temperature and distillation under reduced pressure, the title compound (52.2 mg, 95%) was obtained by column-chromatography using 95:5 mixture of dichloromethane and methanol.

¹H NMR (500 MHz, CDCl₃); δ7.87 (2H, m), 7.51 (1H, m), 7.43 (3H, m), 6.98 (1H, s), 5.35 (2H, s), 4.58 (2H, m), 4.42 (3H, m), 2.74 (2H, m), 1.64 (2H, m), 0.90 (3H, t)

### Example 4-33

### 7-[2-(5-Phenyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine

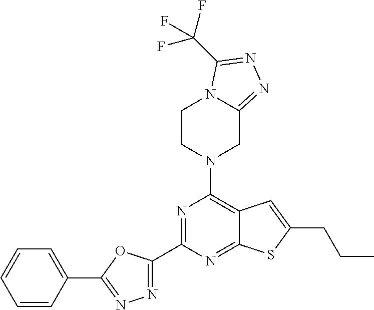

The compound (56.5 mg, 0.11 mmol) obtained from Preparation Example 4-33-1 was suspended in acetonitrile 4 mL. Phosphorous oxychloride (32.7 mg, 0.21 mmol) was added thereto, heated and stirred for 4 hours with reflux. After distillation of the reaction mixture under reduced pressure, the title compound (12.5 mg, 23%) was obtained by column-chromatography using 10:1 mixture of dichloromethane and methanol.

¹H NMR (500 MHz, CDCl₃); δ 8.21 (2H, dd), 7.56 (3H, m), 7.15 (1H, s), 5.43 (2H, s), 4.50 (4H, m), 2.94 (2H, t), 1.82 (2H, m), 1.04 (3H, t)

### Preparation Example 4-34-1

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid N′-(2-cyanoacetyl)hydrazide

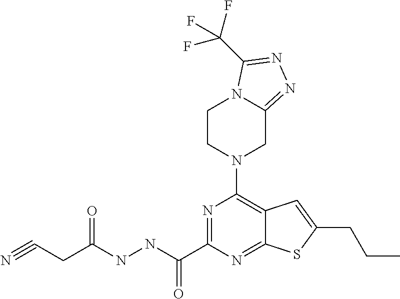

The compound (90.5 mg, 0.22 mmol) obtained from Example 4-2, cyanoacetic acid hydrazide (26.1 mg, 0.26 mmol), EDC (54.7 mg, 0.29 mmol) and HOBT (38.6 mg, 0.29 mmol) were cooled to 0° C. and dissolved in N,N-dimethylformamide 5 mL. Triethylamine (88.8 mg, 0.88 mmol) was added dropwise thereto. After the reaction for 16 hours at room temperature and distillation under reduced pressure, the reaction mixture was diluted with ethyl acetate and washed with water and brine. After the organic layer was dried with anhydrous magnesium sulfate and distilled under reduced pressure, the title compound (41.0 mg, 38%) was obtained by column-chromatography using 10:1 mixture of dichloromethane and methanol.

Mass: M+H 494

### Example 4-34

### {5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetonitrile

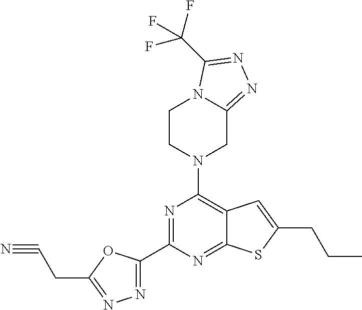

The compound (41 mg, 0.083 mmol) obtained from Preparation Example 4-34-1 was suspended in acetonitrile 4 mL. Phosphorous oxychloride (25.5 mg, 0.17 mmol) was added thereto, heated and stirred for 16 hours with reflux. After cooling of the reaction mixture to room temperature and distillation under reduced pressure, the title compound (3.2 mg, 8%) was obtained by column-chromatography using 10:1 mixture of dichloromethane and methanol.

¹H NMR (500 MHz, CDCl₃); δ 7.18 (1H, s), 5.44 (2H, s), 4.48 (4H, dd), 4.19 (2H, s), 2.96 (2H, t), 1.82 (2H, m), 1.04 (3H, t)

### Preparation Example 4-35-1

### N′-[6-Propyl-4-(3-trifluoromethyl-5,6-dihydro-8H-[1,2,4]triazolo[4,3-a]pyrazin-7-yl)thieno[2,3-d]pyrimidine-2-carbonyl]hydrazinecarboxylic acid tert-butyl ester

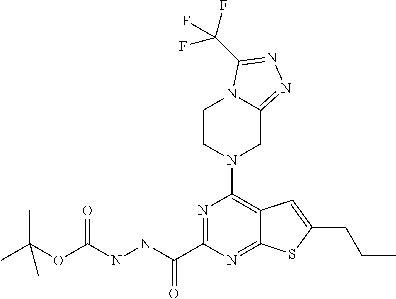

The compound (208.2 mg, 0.5 mmol) obtained from Example 4-2, t-butyl carbazate (80.1 mg, 0.61 mmol), EDC (125.8 mg, 0.66 mmol) and HOBT (88.7 mg, 0.66 mmol) were cooled to 0° C. and dissolved in N,N-dimethylformamide 10 mL. Triethylamine (204 mg, 2.02 mmol) was added dropwise thereto. After the reaction for 16 hours at room temperature and distillation under reduced pressure, the reaction mixture was diluted with ethyl acetate and washed with water and brine. After the organic layer was dried with anhydrous magnesium sulfate and distilled under reduced pressure, the title compound (244.3 mg, 92%) was obtained by column-chromatography using 10:1 mixture of dichloromethane and methanol.

¹H NMR (500 MHz, CDCl₃); δ 10.02 (1H, br s), 7.00 (1H, s), 6.69 (1H, br s), 5.31 (2H, s), 4.41 (4H, s), 2.86 (2H, t), 1.77 (2H, m), 1.53 (9H, s), 1.03 (3H, t)

### Preparation Example 4-35-2

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid N′-(2-methoxyacetyl)hydrazide

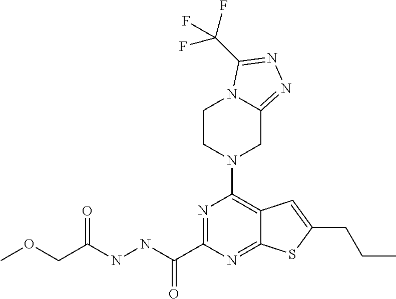

The compound (40 mg, 0.076 mmol) obtained from Preparation Example 4-35-1 was cooled to 0° C., suspended in 4.0 M hydrochloric acid dioxane solution 3 mL and stirred for 3 hours at room temperature. After the solvent was distilled under reduced pressure and dried, methoxyacetic acid (7.5 mg, 0.084 mmol) and HATU (37.6 mg, 0.099 mmol) were added thereto and dissolved in N,N-dimethylformamide 3 mL. The reaction mixture was cooled to 0° C. Triethylamine (30.7 mg, 0.30 mmol) was added dropwise thereto and stirred for 16 hours at room temperature. After distillation of the reaction mixture under reduced pressure, the title compound (36 mg, 95%) was obtained by column-chromatography using 10:1 mixture of dichloromethane and methanol.

Mass: M+H 499

### Example 4-35

### 7-[2-(5-Methoxymethyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine

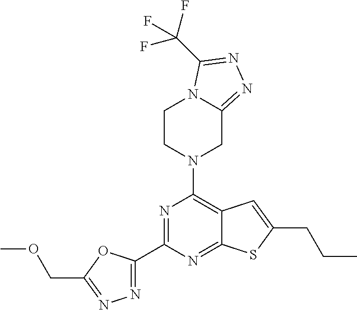

The compound (25.5 mg, 0.05 mmol) obtained from Preparation Example 4-35-2 was suspended in acetonitrile 3 mL. Phosphorous oxychloride (15.7 mg, 0.10 mmol) was added thereto, heated and stirred for 6 hours with reflux. After the reaction mixture was cooled to room temperature and distilled under reduced pressure, the title compound (6.6 mg, 27%) was obtained by column-chromatography using 10:1 mixture of dichloromethane and methanol.

¹H NMR (500 MHz, CDCl₃); δ 7.16 (1H, s), 5.42 (2H, s), 4.77 (2H, s), 4.48 (4H, dd), 3.51 (3H, s), 2.95 (2H, t), 1.81 (2H, m), 1.03 (3H, t)

### Example 4-36

### {5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}methanol

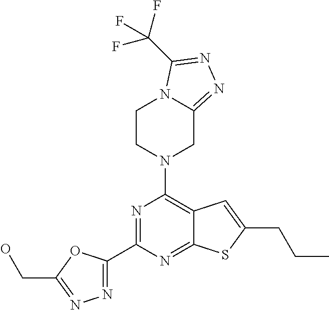

The compound (13.8 mg, 0.029 mmol) obtained from Example 4-35 was dissolved in distilled dichloromethane 3 mL, cooled to −78° C. under N₂ gas and then 1.0 M boron tribromide dichloromethane solution (0.055 mL, 0.055 mmol) was added thereto. The reaction mixture was slowly heated to room temperature, stirred and then cooled to 0° C., again. The reaction was terminated with saturated ammonium chloride aqueous solution. The reaction mixture was diluted with ethyl acetate and washed with brine. After the organic layer was dried with anhydrous magnesium sulfate and distilled under reduced pressure, the title compound (1.6 mg, 12%) was obtained by column-chromatography using 10:1 mixture of dichloromethane and methanol.

¹H NMR (500 MHz, CD₃OD); δ7.48 (1H, s), 5.43 (2H, s), 4.86 (2H, s), 4.49 (4H, s), 2.99 (2H, t), 1.81 (2H, m), 1.04 (3H, t)

### Preparation Example 4-37-1

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid N′-(3-methoxypropionyl)hydrazide

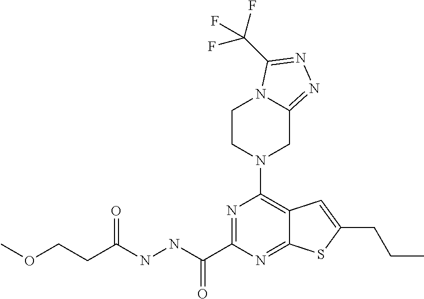

The compound (91.6 mg, 0.17 mmol) obtained from Preparation Example 4-35-1 was cooled to 0° C., suspended in 4.0 M hydrochloric acid dioxane solution 3 mL and stirred for 2 hours at room temperature. After the solvent was distilled under reduced pressure and dried, 3-methoxypropionic acid (19.9 mg, 0.19 mmol) and HATU (86 mg, 0.23 mmol) were added thereto. The reaction mixture was dissolved in N,N-dimethylformamide 4 mL and cooled to 0° C. Triethylamine (70.4 mg, 0.7 mmol) was added dropwise thereto and stirred for 16 hours at room temperature. After distillation of the reaction mixture under reduced pressure, the reaction mixture was diluted with ethyl acetate and washed with brine. After the organic layer was dried with anhydrous magnesium sulfate and distilled under reduced pressure, the title compound (46.3 mg, 52%) was obtained by column-chromatography using 10:1 mixture of dichloromethane and methanol.

Mass: M+H 513

### Example 4-37

### 7-{2-[5-(2-Methoxyethyl)-[1,3,4]oxadiazol-2-yl]-6-propyl-thieno[2,3-d]pyrimidin-4-yl}-3-trifluoromethyl-5,6,7,8-tetrahydro-[1,2,4]triazolo[4,3-a]pyrazine

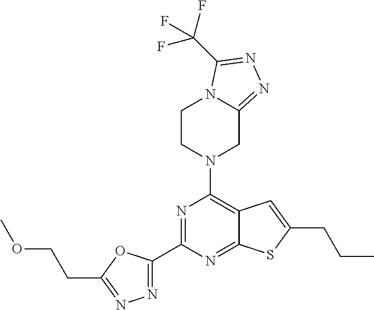

The compound (18.3 mg, 0.036 mmol) obtained from Preparation Example 4-37-1 was suspended in acetonitrile 3 mL. Phosphorous oxychloride (6.02 mg, 0.039 mmol) and N,N-dimethylaniline (4.8 mg, 0.039 mmol) were added thereto, heated and stirred for 16 hours at 80° C. with reflux. After cooling of the reaction mixture to room temperature and distillation of the reaction mixture under reduced pressure, the title compound (11.1 mg, 63%) was obtained by column-chromatography using 95:5 mixture of dichloromethane and methanol.

¹H NMR (500 MHz, CDCl₃); δ 7.14 (1H, s), 5.41 (2H, s), 4.47 (4H, dd), 3.89 (2H, t), 3.39 (3H, s), 3.27 (2H, t), 2.92 (2H, t), 1.80 (2H, m), 1.03 (3H, t)

### Example 4-38

### 2-{5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}ethanol

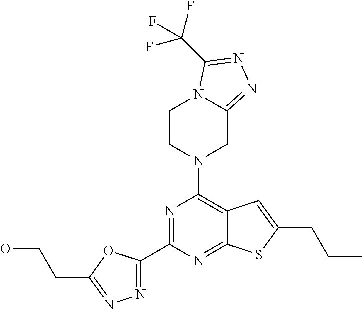

The compound (5.4 mg, 0.011 mmol) obtained from Example 4-37 was dissolved in distilled dichloromethane 3 mL, cooled to −78° C. under N₂ gas and then 1.0 M boron tribromide dichloromethane solution (0.021 mL, 0.021 mmol) was added thereto. The reaction mixture was slowly heated to room temperature, stirred and then cooled to 0° C., again. The reaction was terminated with saturated ammonium chloride aqueous solution. After distillation of the reaction mixture under reduced pressure, formed salt was suspended in 10:1 mixture of dichloromethane and methanol, filtered and removed. After the filtrate was distilled under reduced pressure, the title compound (1.8 mg, 35%) was obtained by column-chromatography using 10:1 mixture of dichloromethane and methanol.

¹H NMR (500 MHz, CDCl₃); δ 7.15 (1H, s), 5.43 (2H, s), 4.47 (4H, m), 4.17 (2H, m), 3.24 (2H, t), 2.95 (2H, t), 2.59 (1H, m), 1.81 (2H, m), 1.04 (3H, t)

### Preparation Example 4-39-1

### 2-({6-propyl-4-[3-(trifluoromethyl)-5,6-dihydro[1,2,4]triazolo[4,3-a]pyrazin-7(8H)-yl]thieno[2,3-d]pyrimidin-2-yl}carbonyl)hydrazinecarboxamide

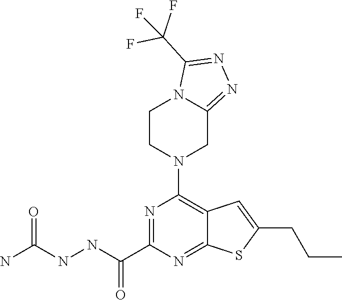

The compound (48.7 mg, 0.12 mmol) obtained from Example 4-2 and carbamic acid hydrazide; hydrochloric acid salt (11.97 mg, 0.107 mmol) were dissolved in N,N-dimethylformamide 4 mL and HATU (53.1 mg, 0.14 mmol) was added thereto. After the reaction mixture was cooled to 0° C., triethylamine (43.4 mg, 0.43 mmol) was added dropwise thereto and stirred for 4 hours at room temperature. After distillation of the reaction mixture under reduced pressure, the title compound (52.7 mg, 95%) was obtained by column-chromatography using 10:1 mixture of dichloromethane and methanol.

¹H NMR (500 MHz, CD₃OD); δ 8.52 (1H, d), 8.20 (1H, d), 7.31 (1H, s), 5.34 (2H, s), 4.47, 4.42 (4H, two m), 3.29 (2H, m), 2.88 (2H, t), 1.72 (2H, m), 0.99 (3H, t)

### Example 4-39

### 5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-ylamine

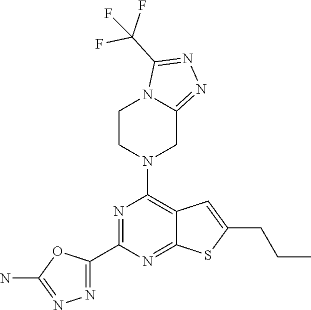

The compound (58 mg, 0.12 mmol) obtained from Preparation Example 4-39-1 was suspended in acetonitrile 4 mL. Phosphorous oxychloride (37.9 mg, 0.25 mmol) was added thereto, heated and stirred for 9 hours with reflux. After cooling of the reaction mixture to room temperature and distillation of the reaction mixture under reduced pressure, the reaction mixture was diluted with ethyl acetate and washed with brine. After the organic layer was dried with anhydrous magnesium sulfate and distilled under reduced pressure, the title compound (1.0 mg, 2%) was obtained by column-chromatography using 10:1 mixture of dichloromethane and methanol.

¹H NMR (500 MHz, CD₃OD); δ 7.44 (1H, s), 5.40 (2H, s), 4.95 (2H, s), 4.46 (4H, m), 2.98 (2H, t), 1.79 (2H, m), 1.02 (3H, t)

### Preparation Example 4-40-1

### 3-Oxo-3-{N′-[6-propyl-4-(3-trifluoromethyl-5,6-dihydro-8H-[1,2,4]triazolo[4,3-a]pyrazin-7-yl)thieno[2,3-d]pyrimidine-2-carbonyl]hydrazino}propionic acid ethyl ester

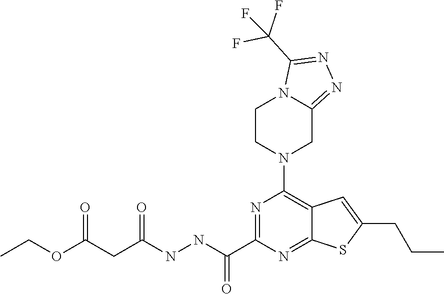

The compound (35.2 mg, 0.067 mmol) obtained from Preparation Example 4-35-1 was cooled to 0° C., suspended in 4.0 M hydrochloric acid dioxane solution 3 mL and stirred for 1 hour at room temperature. After the solvent was distilled under reduced pressure and dried, malonic acid monoethyl ester (9.7 mg, 0.074 mmol) and HATU (33 mg, 0.087 mmol) were added thereto and dissolved in N,N-dimethylformamide 3 mL. The reaction mixture was cooled to 0° C. Triethylamine (27.1 mg, 0.27 mmol) was added dropwise thereto and stirred for 16 hours at room temperature. After distillation of the reaction mixture under reduced pressure, the reaction mixture was diluted with ethyl acetate and washed with brine. After the organic layer was dried with anhydrous magnesium sulfate and distilled under reduced pressure, the title compound (24.2 mg, 67%) was obtained by column-chromatography using 10:1 mixture of dichloromethane and methanol.

Mass: M+H 541

### Example 4-40

### {5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetic acid ethyl ester

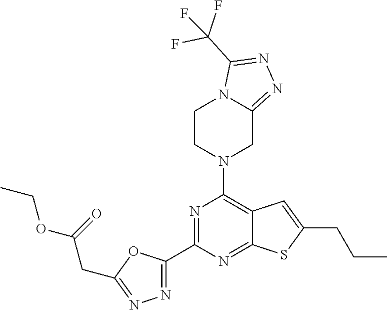

The compound (24.2 mg, 0.045 mmol) obtained from Preparation Example 4-40-1 was suspended in acetonitrile 3 mL. Phosphorous oxychloride (13.7 mg, 0.09 mmol) was added thereto, heated and stirred for 4 hours with reflux. After cooling of the reaction mixture to room temperature and distillation of the reaction mixture under reduced pressure, the title compound (8.0 mg, 34%) was obtained by column-chromatography using 10:1 mixture of dichloromethane and methanol.

¹H NMR (500 MHz, CDCl₃); δ7.15 (1H, s), 5.42 (2H, s), 4.49, 4.46 (4H, two m), 4.23 (2H, q), 4.10 (2H, s), 2.94 (2H, t), 1.81 (2H, m), 1.28 (3H, t), 1.04 (3H, t)

### Example 4-41

### {5-[6-Propyl-4-(3-trifluoromethyl-5,6-dihydro-8H-[1,2,4]triazolo[4,3-a]pyrazin-7-yl)thieno[2,3-d]pyrimidin-2-yl]-[1,3,4]oxadiazol-2-yl}acetic acid

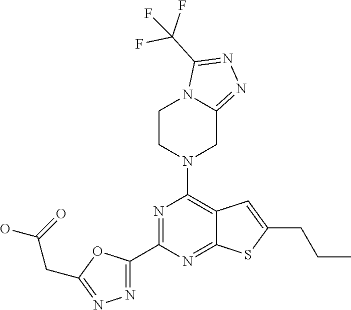

The compound (7.7 mg, 0.015 mmol) obtained from Example 4-40 was dissolved in tetrahydrofuran 1.5 mL, methanol 1 mL and water 0.5 mL. Lithium hydroxide (1.2 mg, 0.03 mmol) was added thereto and the reaction was carried out for 3 hours at room temperature. The reaction mixture was acidified (pH=3) with 1N hydrochloric acid aqueous solution, distilled under reduced pressure, diluted with ethyl acetate and washed with brine. After the organic layer was dried with anhydrous magnesium sulfate and distilled under reduced pressure, the title compound (4 mg, 55%) was obtained by column-chromatography using 85:15 mixture of dichloromethane and methanol.

¹H NMR (500 MHz, CD₃OD); δ 7.45 (1H, s), 5.42 (2H, s), 4.48 (4H, s), 4.02 (2H, s), 2.97 (2H, t), 1.79 (2H, m), 1.03 (3H, t)

### Preparation Example 4-42-1

### 6-Propyl-4-(3-trifluoromethyl-5,6-dihydro-8H-[1,2,4]triazolo[4,3-a]pyrazin-7-yl)-thieno[2,3-d]pyrimidine-2-carboxylic acid N′-phenylacetylhydrazide

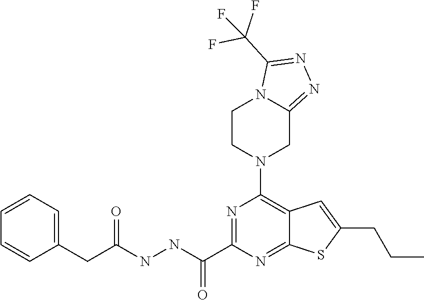

The compound (71.3 mg, 0.14 mmol) obtained from Preparation Example 4-35-1 was cooled to 0° C., suspended in 4.0 M hydrochloric acid dioxane solution 3 mL and stirred for 2 hours at room temperature. After the solvent was distilled under reduced pressure and dried, phenylacetic acid (20.3 mg, 0.15 mmol) and HATU (66.9 mg, 0.18 mmol) were added thereto and dissolved in N,N-dimethylformamide 3 mL. The reaction mixture was cooled to 0° C. Triethylamine (54.8 mg, 0.54 mmol) was added dropwise thereto and stirred for 16 hours at room temperature. The reaction mixture was distilled under reduced pressure, diluted with ethyl acetate and washed with brine. After the organic layer was dried with anhydrous magnesium sulfate and distilled under reduced pressure, the title compound (53.3 mg, 73%) was obtained by column-chromatography using 10:1 mixture of dichloromethane and methanol.

Mass: M+H 545

### Example 4-42

### 7-[2-(5-Benzyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine

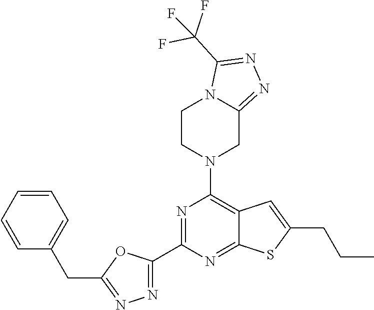

The compound (36 mg, 0.066 mmol) obtained from Preparation Example 4-42-1 was suspended in acetonitrile 4 mL. Phosphorous oxychloride (11.2 mg, 0.073 mmol) was added thereto, heated and stirred for 48 hours at 80° C. with reflux. After cooling of the reaction mixture to room temperature and distillation of the reaction mixture under reduced pressure, the title compound (3.9 mg, 11.2%) was obtained by column-chromatography using 10:1 mixture of dichloromethane and methanol.

¹H NMR (500 MHz, CDCl₃); δ 7.37 (3H, m), 7.29 (2H, m), 7.13 (1H, s), 5.40 (2H, s), 4.43 (4H, m), 4.35 (2H, s), 2.93 (2H, t), 1.80 (2H, m), 1.03 (3H, t)

### Preparation Example 4-43-1

### 6-Propyl-4-(3-trifluoromethyl-5,6-dihydro-8H-[1,2,4]triazolo[4,3-a]pyrazin-7-yl)thieno[2,3-d]pyrimidine-2-carboxylic acid N′-(2-cyclohexylacetyl)hydrazide

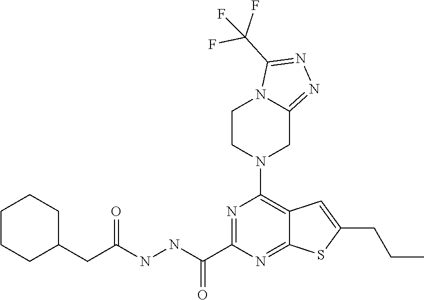

The compound (75.7 mg, 0.14 mmol) obtained from Preparation Example 4-35-1 was cooled to 0° C., suspended in 4.0 M hydrochloric acid dioxane solution 3 mL and stirred for 3 hours at room temperature. After the solvent was distilled under reduced pressure and dried, cyclohexylacetic acid (22.5 mg, 0.158 mmol) and HATU (71.1 mg, 0.19 mmol) were added thereto. The reaction mixture was dissolved in N,N-dimethylformamide 3 mL and cooled to 0° C. Triethylamine (58.2 mg, 0.58 mmol) was added dropwise thereto and stirred for 16 hours at room temperature. The reaction mixture was distilled under reduced pressure, diluted with ethyl acetate and washed with brine. After the organic layer was dried with anhydrous magnesium sulfate and distilled under reduced pressure, the title compound (65.1 mg, 83%) was obtained by column-chromatography using 10:1 mixture of dichloromethane and methanol.

Mass: M+H 551

### Example 4-43

### 7-[2-(5-Cyclohexylmethyl-[1,3,4]oxadiazol-2-yl)-6-propyl-thieno[2,3-d]pyrimidin-4-yl]-3-trifluoromethyl-5,6,7,8-tetrahydro-[1,2,4]triazolo[4,3-a]pyrazine

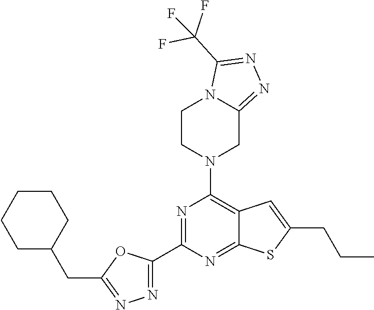

The compound (65.1 mg, 0.12 mmol) obtained from Preparation Example 4-43-1 was suspended in acetonitrile 4 mL. Phosphorous oxychloride (72.5 mg, 0.47 mmol) and N,N-dimethylaniline (57.3 mg, 0.47 mmol) were added thereto, heated and stirred for 16 hours at 80° C. with reflux. After cooling of the reaction mixture to room temperature and distillation of the reaction mixture under reduced pressure, the title compound (35.8 mg, 57%) was obtained by column-chromatography using 95:5 mixture of dichloromethane and methanol.

¹H NMR (500 MHz, CDCl₃); δ7.14 (1H, s), 5.42 (2H, s), 4.47 (4H, m), 2.94 (2H, t), 2.87 (2H, d), 1.95 (1H, m), 1.84˜1.71 (6H, m), 1.68 (1H, m), 1.29˜1.07 (5H, m), 1.04 (3H, t)

### Preparation Example 4-44-1

### 6-Propyl-4-(3-trifluoromethyl-5,6-dihydro-8H-[1,2,4]triazolo[4,3-a]pyrazin-7-yl)-thieno[2,3-d]pyrimidine-2-carbonitrile

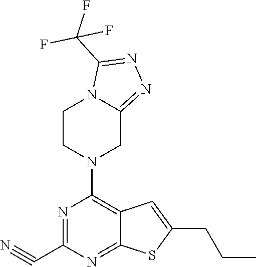

The compound (88 mg, 0.22 mmol) obtained from Preparation Example 1-1-3 and sodium cyanide (21.4 mg, 0.44 mmol) were dissolved in dimethyl sulfoxide 2 mL and water 2 mL. Catalytic amounts of 1,4-diazabicyclo[2,2,2]octane were added thereto, heated and stirred for 16 hours at 90° C. The reaction mixture was cooled to room temperature, diluted with ethyl acetate and washed with water and brine. After the organic layer was dried with anhydrous magnesium sulfate and distilled under reduced pressure, the title compound (68.2 mg, 79.4%) was obtained by column-chromatography using 1:1 mixture of hexane and ethyl acetate.

¹H NMR (500 MHz, CDCl₃); δ 7.15 (1H, s), 5.38 (2H, s), 4.40 (4H, s), 2.95 (2H, t), 1.80 (2H, m), 1.03 (3H, t)

### Example 4-44

### 7-[6-Propyl-2-(1H-tetrazol-5-yl)-thieno[2,3-d]pyrimidin-4-yl]-3-trifluoromethyl-5,6,7,8-tetrahydro-[1,2,4]triazolo[4,3-a]pyrazine

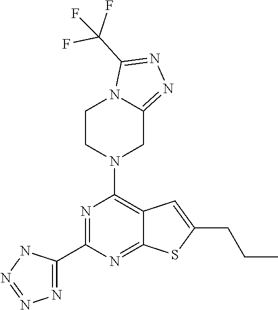

The compound (7.9 mg, 0.02 mmol) obtained from Preparation Example 4-44-1, ammonium chloride (1.1 mg, 0.02 mmol) and sodium azide were heated to 100° C. in microwave reactor. The reaction was carried out for 5 hours. The reaction mixture was cooled to room temperature and suspended in 10:1 mixture of dichloromethane and methanol and filtered to obtain the filtrate. The title compound (7.8 mg, 87%) was obtained by column-chromatography using 10:1 mixture of dichloromethane and methanol.

¹H NMR (500 MHz, CD₃OD); δ 7.36 (1H, s), 5.43 (2H, s), 4.50 (4H, s), 2.95 (2H, t), 1.80 (2H, m), 1.03 (3H, t)

### Preparation Example 4-45

### 6-Propyl-4-(3-trifluoromethyl-5,6-dihydro-8H-[1,2,4]triazolo[4,3-a]pyrazin-7-yl)-thieno[2,3-d]pyrimidine-2-carbothioic acid amide

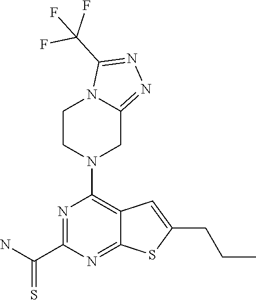

The compound (62.4 mg, 0.15 mmol) obtained from Example 4-3 and Lawesson's reagent (67.5 mg, 0.17 mmol) were suspended in benzene 4 mL, and stirred for 16 hours with reflux. After distillation under reduced pressure, the reaction mixture was diluted with ethyl acetate and washed with water and brine. After the organic layer was dried with anhydrous magnesium sulfate and distilled under reduced pressure, the title compound (39.5 mg, 61%) was obtained by column-chromatography using 90:10 mixture of dichloromethane and methanol.

¹H NMR (400 MHz, CDCl₃); δ 9.15 (1H, br), 7.73 (1H, br), 7.12 (1H, s), 5.36 (2H, s), 4.46 (4H, br), 2.93 (2H, t), 1.79 (2H, m), 1.03 (3H, t)

### Example 4-45

### 2-[6-Propyl-4-(3-trifluoromethyl-5,6-dihydro-8H-[1,2,4]triazolo[4,3-a]pyrazin-7-yl)thieno[2,3-d]pyrimidin-2-yl]thiazole-4-carboxylic acid ethyl ester

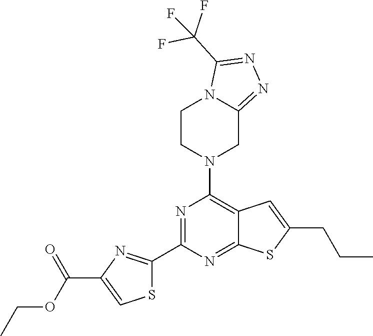

The compound (20.9 mg, 0.049 mmol) obtained from Preparation Example 4-45 was dissolved in dioxane 4 mL. 3-bromo-2-oxo-propionic acid ethyl ester (9.1 mg, 0.046 mmol) was added thereto, heated and stirred for 16 hours with reflux. After cooling of the reaction mixture to room temperature and distillation of the reaction mixture under reduced pressure, the title compound (14.5 mg, 57%) was obtained by column-chromatography using 90:10 mixture of dichloromethane and methanol.

¹H NMR (500M z, CDCl₃); δ 8.30 (1H, s), 7.07 (1H, s), 5.37 (2H, s), 4.45 (6H, m), 2.89 (2H, t), 1.77 (2H, m), 1.42 (3H, t), 1.01 (3H, t)

### Example 4-46

### 2-[6-Propyl-4-(3-trifluoromethyl-5,6-dihydro-8H-[1,2,4]triazolo[4,3-a]pyrazin-7-yl)thieno[2,3-d]pyrimidin-2-yl]thiazole-4-carboxylic acid

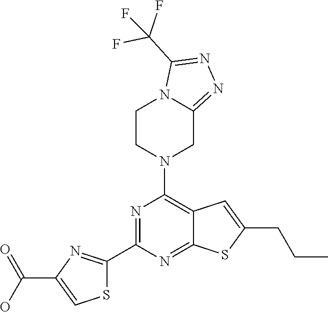

The compound (12.4 mg, 0.024 mmol) obtained from Example 4-45 was dissolved in tetrahydrofuran 1.5 mL, methanol 1 mL and water 0.5 mL. Lithium hydroxide (1.5 mg, 0.036 mmol) was added thereto and the reaction was carried out for 4 hours at room temperature. The reaction mixture was acidified (pH=3) with 1N hydrochloric acid aqueous solution, distilled under reduced pressure, diluted with ethyl acetate and washed with brine. After the organic layer was dried with anhydrous magnesium sulfate and distilled under reduced pressure, the title compound (7.3 mg, 62%) was obtained by column-chromatography using 85:15 mixture of dichloromethane and methanol.

¹H NMR (500 MHz, MeOD); δ 8.21 (1H, s), 7.36 (1H, s), 5.34 (2H, s), 4.45 (4H, br), 2.92 (2H, t), 1.76 (2H, m), 1.01 (3H, t)

### Preparation Example 4-47-1

### (R)-2-Amino-3-(4-methoxy-benzylsulfanyl)propionic acid methyl ester

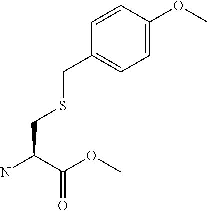

(R)-2-amino-3-(4-methoxy-benzylsulfanyl)propionic acid (270.1 mg, 1.12 mmol) was dissolved in methanol 5 mL and cooled to 0° C. Trimethylsilyl chloride (364.8 mg, 3.36 mmol) was slowly added dropwise thereto. The reaction mixture was stirred for 16 hours at room temperature, diluted with ethyl acetate and washed with saturated sodium hydrogen carbonate aqueous solution and brine. The title compound (178.8 mg, 63%) was obtained by drying the organic layer with anhydrous magnesium sulfate and distillation under reduced pressure.

¹H NMR (400M z, CDCl₃); δ 7.27 (2H, d), 6.88 (2H, d), 3.84 (3H, s), 3.77 (3H, s), 3.74 (2H, s), 3.63 (1H, q), 2.85 (1H, m), 2.68 (1H, m)

### Preparation Example 4-47-2

### (R)-3-(4-Methoxy-benzylsulfanyl)-2-{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}propionic acid methyl ester

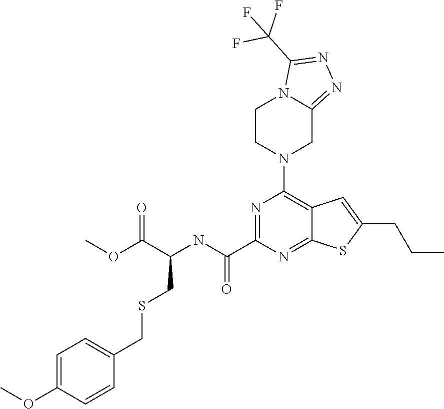

The compound (134.7 mg, 0.33 mmol) obtained from Example 4-2 and the compound (75.8 mg, 0.3 mmol) obtained from Preparation Example 4-47-1 were dissolved in N,N-dimethylformamide 4 mL. HATU (146.7 mg, 0.39 mmol) was added thereto and the reaction mixture was cooled to 0° C. Triethylamine (120.2 mg, 1.19 mmol) was added dropwise thereto and stirred for 16 hours at room temperature. After distillation under reduced pressure, the reaction mixture was diluted with ethyl acetate and washed with water and brine. After the organic layer was dried with anhydrous magnesium sulfate and distilled under reduced pressure, the title compound (191 mg, 99.0%) was obtained by column-chromatography using 93:7 mixture of dichloromethane and methanol.

¹H NMR (400 MHz, CDCl₃); δ 8.61 (1H, d), 7.17 (2H, d), 7.11 (1H, s), 6.74 (2H, d), 5.30 (2H, s), 4.97 (1H, m), 4.41 (4H, br), 3.77 (3H, s), 3.69 (5H, br), 3.03 (2H, m), 2.91 (2H, t), 1.75 (2H, m), 0.99 (3H, t)

### Example 4-47

### (R)-2-[6-Propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazole-4-carboxylic acid methyl ester

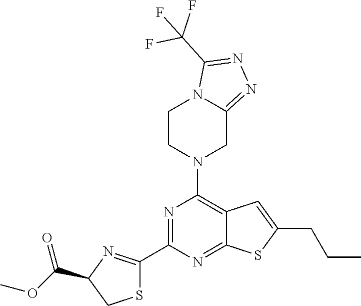

The compound (191 mg, 0.29 mmol) obtained from Preparation Example 4-47-2 was dissolved in dichloromethane 7 mL. Phosphorous pentachloride (122.4 mg, 0.59 mmol) was added dropwise thereto and stirred for 16 hours at room temperature. The reaction mixture was diluted with ethyl acetate and washed with water and brine. After the organic layer was dried with anhydrous magnesium sulfate and distilled under reduced pressure, the title compound (48.4 mg, 32.3%) was obtained by column-chromatography using 93:7 mixture of dichloromethane and methanol.

¹H NMR (500 MHz, CDCl₃); δ 7.07 (1H, s), 5.39 (1H, t), 5.34 (2H, s), 4.43 (2H, br), 4.37 (2H, d), 3.79 (3H, s), 3.67 (1H, t), 3.61 (1H, t), 2.89 (2H, t), 1.79 (2H, m), 1.00 (3H, t)

### Preparation Example 4-48-1

### (S)-2-tert-Butoxycarbonylamino-succinic acid 4-isopropyl ester 1-methyl ester

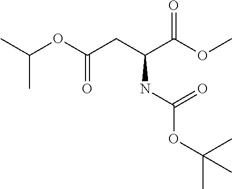

(S)-2-t-Butoxycarbonylamino-succinic acid 1-methyl ester (76.7 g, 310 mmol) was dissolved in dichloromethane 770 mL. Isopropyl alcohol (37.3 g, 620 mmol) and 4-N,N-dimethylaminopyridine (49.2 g, 403 mmol) were added thereto and cooled to 0° C. Then, EDC (71.3 g, 372 mmol) was added thereto, stirred for 16 hours at room temperature and washed two times with 0.5N hydrochloric acid. The title compound (88 g, 98%) was obtained by drying the organic layer with anhydrous magnesium sulfate and distillation under reduced pressure.

¹H NMR (400 MHz, CDCl₃); δ 5.50 (1H, br), 5.00 (1H, m), 4.58 (1H, br), 3.45 (3H, s), 2.90 (1H, q), 2.77 (1H, q), 1.47 (9H, s), 1.23 (6H, d)

### Preparation Example 4-48-2

### (S)-3-tert-Butoxycarbonylamino-4-hydroxy-butyric acid isopropyl ester

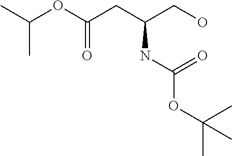

The compound (88 g, 304 mmol) obtained from Preparation Example 4-48-1 was dissolved in methanol 633 mL and cooled to 0° C. Sodium borohydride (23 g, 608 mmol) was slowly added thereto. After stirring for 30 minutes at 0° C., the reaction was terminated with 10% ammonium chloride aqueous solution and 1N hydrochloric acid was added thereto. The extraction was carried out two times using 1:3 mixture of toluene and ethyl acetate. After the organic layer was dried with anhydrous magnesium sulfate and distilled under reduced pressure, the title compound (60.6 mg, 76%) was obtained by column-chromatography using 1:1 mixture of hexane and ethyl acetate.

¹H NMR (400 MHz, CDCl₃); δ 5.34 (1H, br), 5.12 (1H, m), 4.11 (1H, m), 3.84 (2H, d), 2.69 (2H, d), 1.58 (9H, s), 1.38 (6H, d)

### Preparation Example 4-48-3

### (S)-3-tert-Butoxycarbonylamino-4-(4-methoxy-benzylsulfanyl)butyric acid isopropyl ester

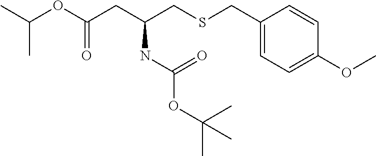

The compound (60.6 g, 232 mmol) obtained from Preparation Example 4-48-2 was dissolved in dichloromethane 600 mL and cooled to 0° C. Methanesulfanylchloride (29.2 g, 255 mmol) was slowly added thereto. After stirring for 1 hour at 0° C., the reaction mixture was diluted with dichloromethane and washed with saturated sodium hydrogen carbonate aqueous solution and brine. After the organic layer was dried with anhydrous magnesium sulfate, distilled under reduced pressure and added in the solution which was prepared by adding (4-methoxyphenyl)methanethiol (42.9 g, 278 mmol) in N,N-dimethylformamide 500 mL and adding sodium hydride (6.67 g, 278 mmol) at −20° C., the reaction mixture was stirred for 3 hours at room temperature and distilled under reduced pressure. Then, the reaction mixture was diluted with ethyl acetate and washed with saturated ammonium chloride aqueous solution. After the reaction mixture was dried with anhydrous magnesium sulfate and distilled under reduced pressure, the title compound (54.1 g, 59%) was obtained by column-chromatography using 5:1 mixture of hexane and ethyl acetate.

¹H NMR (400 MHz, CDCl₃); δ 7.25 (2H, d), 6.87 (2H, d), 5.13 (1H, br), 5.01 (1H, p), 4.14 (1H, q), 3.83 (3H, s), 3.74 (2H, s), 2.58 (4H, m), 1.49 (9H, s), 1.28 (3H, s), 1.26 (6H, d)

### Preparation Example 4-48-4

### (S)-3-Amino-4-(4-methoxy-benzylsulfanyl)butyric acid isopropyl ester; hydrochloride

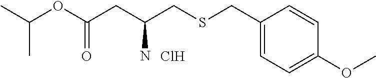

The compound (54.1 g, 136 mmol) obtained from Preparation Example 4-48-3 was dissolved in diethylether 150 mL and cooled to 0° C. 4.0 N hydrochloric acid diethyl ether 237 mL was added thereto. The title compound (43.2 g, 100%) was obtained by stirring for 1 hour at room temperature and distillation under reduced pressure.

Mass: M+H 333

### Preparation Example 4-48-5

### (R)-4-(4-Methoxy-benzylsulfanyl)-3-{[6-propyl-4-(3-trifluoromethyl-5,6-dihydro-8H-[1,2,4]triazolo[4,3-a]pyrazin-7-yl)thieno[2,3-d]pyrimidine-2-carbonyl]amino}-butyric acid isopropyl ester

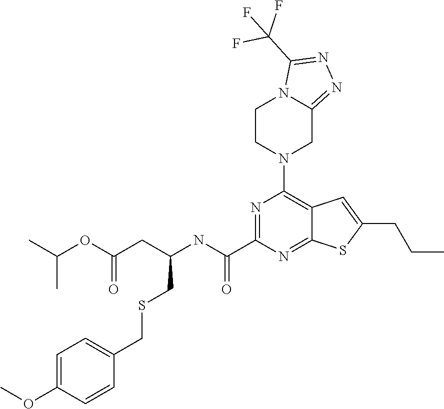

The compound (300 mg, 0.73 mmol) obtained from Example 4-2, the compound (220.8 mg, 0.66 mmol) obtained from Preparation Example 4-48-4, EDC (164.8 mg, 0.86 mmol) and HOBT 116.2 mg (0.86 mmol) were cooled to 0° C. and dissolved in N,N-dimethylformamide 10 mL. Triethylamine (267.7 mg, 2.65 mmol) was added thereto. The reaction mixture was stirred for 16 hours at room temperature, distilled under reduced pressure, diluted with ethyl acetate and successively washed with saturated sodium hydrogen carbonate aqueous solution, 1 N hydrochloric acid and brine. The title compound (438 g, 96%) was obtained by drying the organic layer with anhydrous magnesium sulfate and distillation under reduced pressure.

Mass: M+H 691

### Example 4-48

### {(R)-2-[6-Propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazol-4-yl}acetic acid isopropyl ester

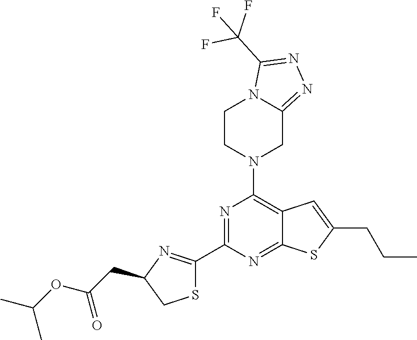

The compound (438 mg, 0.63 mmol) obtained from Preparation Example 4-48-5 was dissolved in dichloromethane 20 mL. Phosphorous pentachloride (263.7 mg, 1.27 mmol) was added dropwise thereto and stirred for 16 hours at room temperature. After the reaction mixture was poured into diethyl ether, formed solid compound was filtered and dried. The title compound (172.2 mg, 49%) was obtained by column-chromatography using 95:5 mixture of dichloromethane and methanol.

¹H NMR (500 MHz, CDCl₃); δ 7.04 (1H, s), 5.35 (2H, s), 5.07 (1H, m), 5.05 (1H, m), 4.44 (2H, d), 4.37 (2H, d), 3.61 (1H, t), 3.16 (2H, m), 2.90 (2H, t), 2.67 (1H, m), 1.78 (6H, m), 1.25 (2H, d), 1.02 (3H, t)

### Example 4-49

### {(R)-2-[6-Propyl-4-(3-trifluoromethyl-5,6-dihydro-8H-[1,2,4]triazolo[4,3-a]pyrazin-7-yl)thieno[2,3-d]pyrimidin-2-yl]-4,5-dihydro-thiazol-4-yl}acetic acid

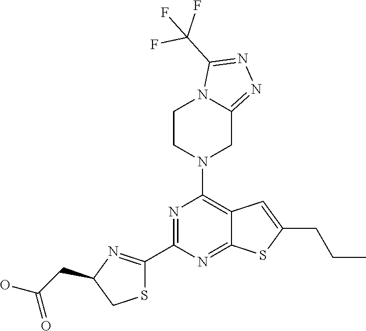

The compound (36.7 mg, 0.068 mmol) obtained from Example 4-48 was dissolved in tetrahydrofuran 1.5 mL, methanol 1 mL and water 0.5 mL. Lithium hydroxide (4.3 mg, 0.1 mmol) was added thereto and the reaction was carried out for 4 hours at room temperature. After the reaction mixture was acidified (pH=3) with 1 N hydrochloric acid aqueous solution and distilled under reduced pressure, the title compound (18 mg, 53%) was obtained by column-chromatography using 85:15 mixture of dichloromethane and methanol.

¹H NMR (500 MHz, MeOD); δ 7.42 (1H, s), 5.38 (2H, s), 5.22 (H, br), 4.45 (4H, br), 4.09 (1H, t), 3.50 (1H, t), 2.90 (2H, t), 2.85 (1H, br), 2.75 (1H, br), 1.71 (2H, m), 0.94 (3H, t)

### Example 4-50

### {2-[6-Propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}methanol

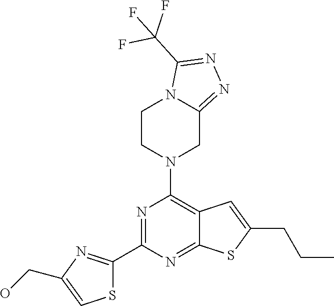

The compound (57.6 mg, 0.11 mmol) obtained from Example 4-45 was dissolved in distilled tetrahydrofuran 4 mL and cooled to 0° C. Lithium borohydride (2.0 M tetrahydrofuran solution) (0.11 mL, 0.22 mmol) was slowly added dropwise thereto and the reaction was carried out for 2 hours at room temperature. The reaction was terminated with saturated sodium hydrogen carbonate aqueous solution and the extraction was carried out with ethyl acetate, followed by washing with brine. After the organic layer was dried with anhydrous magnesium sulfate and distilled under reduced pressure, the title compound (4.6 mg, 9%) was obtained by column-chromatography using 95:5 mixture of dichloromethane and methanol.

¹H NMR (500 MHz, CDCl₃); δ 7.39 (1H, s), 7.06 (1H, s), 5.37 (2H, s), 4.90 (2H, d), 4.46 (2H, d), 4.40 (2H, d), 2.89 (2H, t), 2.40 (1H, t), 1.77 (2H, m), 1.01 (3H, t)

### Example 4-51

### {2-[6-Propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}acetic acid ethyl ester

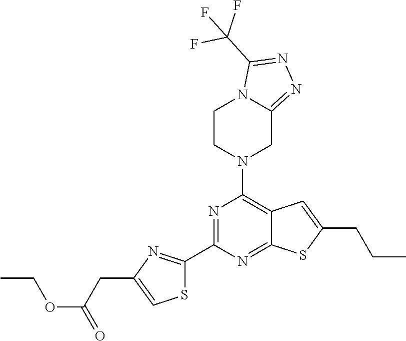

The compound (30.4 mg, 0.071 mmol) obtained from Preparation Example 4-45 was dissolved in ethanol 5 mL. Ethyl 4-chloroacetoacetate (11.8 mg, 0.071 mmol) was added thereto, heated and stirred for 16 hours with reflux. After cooling of the reaction mixture to room temperature and distillation of the reaction mixture under reduced pressure, the column-chromatography using 95:5 mixture of dichloromethane and methanol was carried out. The title compound (11.5 mg, 30%) was obtained by additional column-chromatography using 1:1 mixture of hexane and ethyl acetate.

¹H NMR (500 MHz, CDCl₃); δ 7.40 (1H, s), δ 7.03 (1H, s), 5.32 (2H, s), 4.44 (2H, d), 4.37 (2H, d), 4.18 (2H, q), 4.00 (2H, s), 2.87 (2H, t), 1.76 (2H, m), 1.26 (3H, t), 1.02 (3H, t)

### Example 4-52

### {2-[6-Propyl-4-(3-trifluoromethyl-5,6-dihydro-8H-[1,2,4]triazolo[4,3-a]pyrazin-7-yl)thieno[2,3-d]pyrimidin-2-yl]thiazol-4-yl}acetic acid

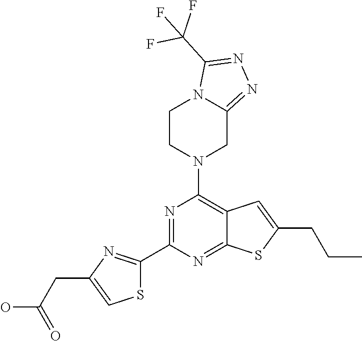

The compound (10.4 mg, 0.019 mmol) obtained from Example 4-51 was dissolved in tetrahydrofuran 1.5 mL, methanol 1 mL and water 0.5 mL. Lithium hydroxide (1.62 mg, 0.039 mmol) was added thereto and the reaction was carried out for 4 hours at room temperature. The reaction mixture was acidified (pH=3) with 1 N hydrochloric acid aqueous solution and distilled under reduced pressure. The title compound (8.3 mg, 84%) was obtained by column-chromatography using 85:15 mixture of dichloromethane and methanol.

¹H NMR (500 MHz, MeOD); δ 7.9 (1H, br), 7.34 (1H, s), 5.33 (2H, s), 4.40 (4H, br), 3.96 (2H, br), 2.84 (2H, br), 1.65 (2H, br), 0.95 (3H, br)

### Preparation Example 4-53-1

### (4-Hydroxy-6-propyl-thieno[2,3-d]pyrimidin-2-yl)acetic acid ethyl ester

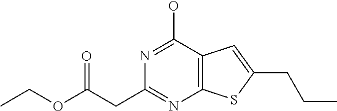

The compound (2.28 g, 11.4 mmol) obtained from Preparation Example 4-1-1 and ethyl cyanoacetate (1.29 g, 11.4 mmol) were dissolved in dioxane 10 mL and cooled to 0° C. 4.0 N hydrochloric acid dioxane solution 11 mL was slowly added dropwise thereto and stirred for 16 hours at room temperature. After the solvent was distilled under reduced pressure for removal, the remaining residue was diluted with water, basified with saturated sodium hydrogen carbonate aqueous solution and extracted several times with ethyl acetate. After the organic layer was dried with anhydrous magnesium sulfate and distilled under reduced pressure, the title compound (1.69 g, 53%) was obtained by column-chromatography using 7:3-1:1 mixture of hexane and ethyl acetate.

¹H NMR (400 MHz, CDCl₃); δ11.04 (1H, br), 7.14 (1H, s), 4.24 (2H, q), 3.80 (2H, s), 2.80 (2H, t), 1.70 (2H, m), 1.29 (3H, t), 0.98 (3H, t)

### Preparation Example 4-53-2

### (4-Chloro-6-propyl-thieno[2,3-d]pyrimidin-2-yl)acetic acid ethyl ester

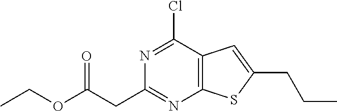

The compound (1.69 g, 6.03 mmol) obtained from Preparation Example 4-53-1 was suspended in phosphorous oxychloride 20 mL and stirred for 6 hours with reflux. After the reaction mixture was distilled under reduced pressure, the title compound (1.65 g, 92%) was obtained by column-chromatography using 7:3 mixture of hexane and ethyl acetate.

¹H NMR (400 MHz, CDCl₃); δ7.07 (1H, s), 4.19 (2H, q), 4.06 (2H, s), 2.90 (2H, t), 1.76 (2H, m), 1.24 (3H, t), 1.01 (3H, t)

### Preparation Example 4-53

### [6-Propyl-4-(3-trifluoromethyl-5,6-dihydro-8H-[1,2,4]triazolo[4,3-a]pyrazin-7-yl)thieno[2,3-d]pyrimidin-2-yl]acetic acid ethyl ester

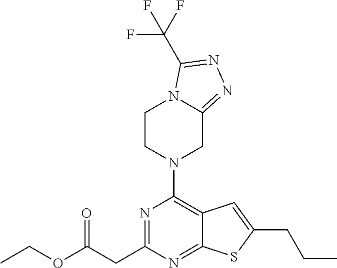

The compound (1.65 g, 5.51 mmol) obtained from Preparation Example 4-53-2 and the compound (1.26 g, 5.51 mmol) obtained from Preparation Example 1-1-2 were diluted with N,N-dimethylformamide 20 mL. Triethylamine (1.67 g, 16.5 mmol) was added thereto at 0° C. and stirred for 16 hours at room temperature. Since the reaction was not completed, the reaction was carried out for 4 days at 60° C., followed by cooling to room temperature and distillation under reduced pressure. The remaining residue was diluted with ethyl acetate and washed with water and brine. After the organic layer was dried with anhydrous magnesium sulfate and distilled under reduced pressure, the title compound (1.74 g, 70%) was obtained by column-chromatography using 1:1 mixture of hexane and ethyl acetate, and 95:5 mixture of dichloromethane and methanol.

¹H NMR (500 MHz, CDCl₃); δ 7.00 (1H, s), 5.29 (2H, s), 4.36 (2H, d), 4.30 (2H, d), 4.19 (2H, q), 3.88 (2H, s), 2.86 (2H, t), 1.75 (2H, m), 1.25 (3H, t), 1.00 (3H, t)

### Example 4-54

### [6-Propyl-4-(3-trifluoromethyl-5,6-dihydro-8H-[1,2,4]triazolo[4,3-a]pyrazin-7-yl)thieno[2,3-d]pyrimidin-2-yl]acetic acid

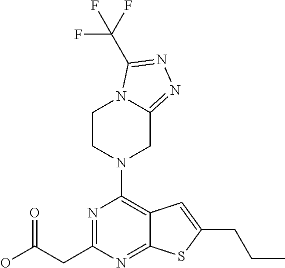

The compound (1.7 g, 3.75 mmol) obtained from Example 4-53 was dissolved in tetrahydrofuran 18 mL, methanol 12 mL and water 6 mL. Lithium hydroxide (315 mg, 7.5 mmol) was added thereto and the reaction was carried out for 4 hours at room temperature. The reaction mixture was distilled under reduced pressure, diluted with ethyl acetate and washed with water for removal of impurities. After the aqueous layer was acidified (pH=3) with 1 N hydrochloric acid aqueous solution, ethyl acetate was added thereto to form the precipitate. The title compound (1.41 g, 88%) was obtained by filtration and drying of formed precipitate.

¹H NMR (500 MHz, DMSO, d₆); δ 12.45 (1H, br) 7.46 (1H, s), 5.19 (2H, s), 4.34 (2H, d), 4.26 (2H, d), 3.71 (2H, s), 2.85 (2H, t), 1.66 (2H, m), 0.91 (3H, t)

### Example 4-55

### 2-[6-Propyl-4-(3-trifluoromethyl-5,6-dihydro-8H-[1,2,4]triazolo[4,3-a]pyrazin-7-yl)-thieno[2,3-d]pyrimidin-2-yl]-acetamide

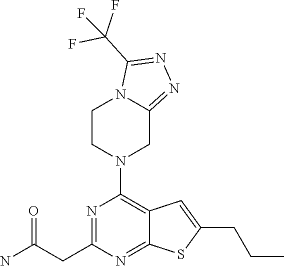

The compound (500 mg, 1.17 mmol) obtained from Example 4-54, ammonium chloride (70 mg, 1.29 mmol), EDC (270 mg, 1.41 mmol) and HOBT (238 mg, 0.18 mmol) was dissolved in N,N-dimethylformamide 40 mL and cooled to 0° C. Diisopropyl ethylamine (759 mg, 5.87 mmol) was added dropwise thereto. The reaction was carried out for 16 hours at room temperature, followed by distillation under reduced pressure. The remaining residue was diluted with ethyl acetate and washed with water and brine. After the organic layer was dried with anhydrous magnesium sulfate and distilled under reduced pressure, the title compound (430 mg, 86%) was obtained by column-chromatography using 95:5 mixture of dichloromethane and methanol.

¹H NMR (400 MHz, DMSO, d₆); δ 7.70 (1H, s), 7.40 (1H, br), 6.99 (1H, br), 5.23 (2H, s), 4.38 (2H, d), 4.30 (2H, d), 3.60 (2H, s), 2.88 (2H, t), 1.67 (2H, m), 0.94 (3H, t)

### Example 4-56

### 2-[6-Propyl-4-(3-trifluoromethyl-5,6-dihydro-8H-[1,2,4]triazolo[4,3-a]pyrazin-7-yl)-thieno[2,3-d]pyrimidin-2-yl]-thioacetamide

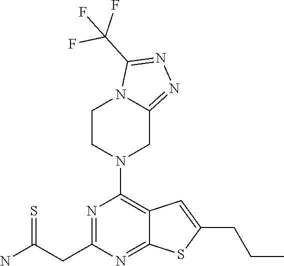

The compound (400 mg, 0.94 mmol) obtained from Example 4-55, Lawesson s reagent (457 mg, 1.13 mmol) were added in benzene 20 mL and stirred for 18 hours with reflux. The reaction solution was distilled under reduced pressure, diluted with ethyl acetate and washed with water. After the organic layer was dried with anhydrous magnesium sulfate and distilled under reduced pressure, the title compound (285 mg, 69%) was obtained by column-chromatography using 90:10 mixture of dichloromethane and methanol.

¹H NMR (400 MHz, MeOD); δ 7.35 (1H, s), 5.33 (2H, s), 4.45 (2H, d), 4.39 (2H, d), 3.80 (2H, s), 2.95 (2H, t), 1.79 (2H, m), 1.03 (3H, t)

### Example 4-57

### 2-[6-Propyl-4-(3-trifluoromethyl-5,6-dihydro-8H-[1,2,4]triazolo[4,3-a]pyrazin-7-yl)-thieno[2,3-d]pyrimidin-2-ylmethyl]-thiazole-4-carboxylic acid ethyl ester

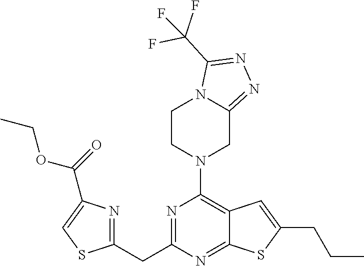

The compound (180 mg, 0.41 mmol) obtained from Example 4-56 and 3-bromo-2-oxo-propionic acid ethyl ester (79 mg, 0.41 mmol) was dissolved in dioxane 10 mL and stirred for 13 hours with reflux. The reaction solution was distilled under reduced pressure, diluted with ethyl acetate and washed with water and saturated sodium hydrogen carbonate aqueous solution. After the organic layer was dried with anhydrous magnesium sulfate and distilled under reduced pressure, the title compound (70 mg, 33%) was obtained by column-chromatography using 95:5 mixture of dichloromethane and methanol.

¹H NMR (400 MHz, CDCl₃); δ8.12 (1H, s), 7.01 (1H, s), 5.29 (2H, s), 4.67 (2H, s), 4.41 (2H, q), 4.29 (4H, br), 2.87 (2H, t), 1.73 (2H, m), 1.39 (3H, t), 1.01 (3H, t)

### Example 4-58

### 2-[6-Propyl-4-(3-trifluoromethyl-5,6-dihydro-8H-[1,2,4]triazolo[4,3-a]pyrazin-7-yl)-thieno[2,3-d]pyrimidin-2-ylmethyl]-thiazole-4-carboxylic acid

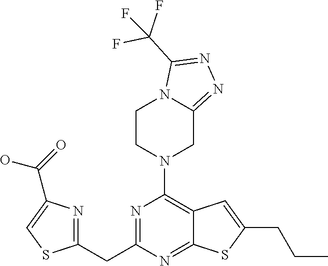

The compound (25 mg, 0.048 mmol) obtained from Example 4-57 was dissolved in 3:2:1 mixture of tetrahydrofuran, water and methanol. Lithium hydroxide (4 mg, 0.096 mmol) was added thereto and stirred for 5 hours at room temperature. The reaction mixture was acidified (pH=4) with 1 N hydrochloric acid aqueous solution and distilled under reduced pressure. The title compound (5 mg, 21%) was obtained by column-chromatography using 90:10 mixture of dichloromethane and methanol.

¹H NMR (400 MHz, MeOD); δ7.80 (1H, br), 7.21 (1H, s), 5.13 (2H, br), 4.57 (2H, br), 4.37 (2H, br), 4.25 (2H, br), 2.82 (2H, t), 1.67 (2H, m), 0.95 (3H, t)

### Example 4-59

### 2-{2-[6-Propyl-4-(3-trifluoromethyl-5,6-dihydro-8H-[1,2,4]triazolo[4,3-a]pyrazin-7-yl)-thieno[2,3-d]pyrimidin-2-yl]-thiazol-4-yl}-ethanol

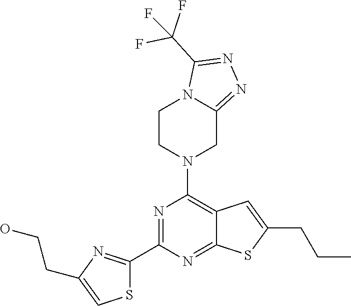

Similar to the method described in Example 4-50, the title compound (7 mg, 17%) was obtained by using the compound (45 mg, 0.084 mmol) obtained from Example 4-51 and lithium borohydride (2.0 M tetrahydrofuran solution) (0.084 mL, 0.17 mmol).

¹H NMR (400 MHz, DMSO, d₆); δ7.20 (1H, s), 7.03 (1H, s), 5.35 (2H, s), 4.45 (2H, d), 4.39 (2H, d), 4.03 (2H, t), 3.06 (2H, t), 2.85 (2H, t), 1.76 (2H, m), 1.02 (3H, t)

### Preparation Example 5-1-1

### Cyano-propionylamino-acetic acid ethyl ester

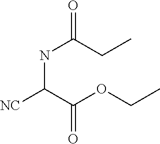

Cyano-hydroxyimino-acetic acid ethyl ester (20 g, 140 mmol) was dissolved in sodium hydrogen carbonate aqueous solution 160 mL and water 200 mL. Sodium thiosulfate (73.5 g, 422 mmol) was added thereto and stirred for 1 hour at 40° C. Brine 250 mL was added to the reaction mixture and the extraction was carried out 4 times with dichloromethane 500 mL. After the organic layer was dried with anhydrous magnesium sulfate and distilled under reduced pressure, amino-cyano-acetic acid ethyl ester (6.37 g, 35%) was obtained, dissolved in dichloromethane 150 mL and cooled to 0° C. Pyridine (3.93 g, 49.7 mmol) and propionyl chloride (4.6 g, 49.7 mmol) was added thereto and stirred for 12 hours at room temperature. Dichloromethane 250 mL was added in the reaction mixture and washed two times with water 200 mL. After the organic layer was dried with anhydrous magnesium sulfate and distilled under reduced pressure, the title compound (3.93 g, 43%) was obtained by recrystallization in the mixture of ether and anhydrous magnesium sulfate.

¹H NMR (400 MHz, CDCl₃); δ 6.33 (1H, d), 5.54 (1H, d), 4.37 (2H, g), 2.35 (2H, q), 1.37 (3H, t), 1.20 (3H, t)

### Preparation Example 5-1-2

### 5-Amino-2-ethyl-thiazole-4-carboxylic acid ethyl ester

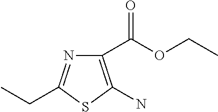

The compound (4.37 g, 23.7 mmol) obtained from Preparation Example 5-1-1 was dissolved in benzene 60 mL. Anhydrous Lawesson's reagent (4.8 g, 11.9 mmol) was added thereto and stirred for 12 hours with reflux. After the reaction mixture was distilled under reduced pressure, the title compound (2.61 g, 55%) was obtained by column-chromatography using 95:5 mixture of dichloromethane and methanol.

¹H NMR (400 MHz, CDCl₃); δ5.92 (2H, br s), 4.38 (2H, q), 2.87 (2H, q), 1.40 (3H, t), 1.30 (3H, t)

### Preparation Example 5-1-3

### 2-Ethyl-5-phenoxycarbonylamino-thiazole-4-carboxylic acid ethyl ester

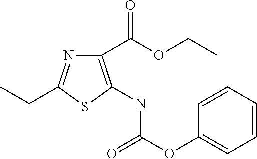

The compound (0.19 g, 0.95 mmol) obtained from Preparation Example 5-1-2 was dissolved in dichloromethane 10 mL. Pyridine (0.12 g, 1.42 mmol) and phenyl chloroformate (0.16 g, 1.04 mmol) were added thereto and stirred for 48 hours. The reaction mixture was added in water 15 mL and the extraction was carried out three times with dichloromethane 25 mL. After the organic layer was dried with anhydrous magnesium sulfate and distilled under reduced pressure, the title compound (265 mg, 87%) was obtained by column-chromatography using 90:10 mixture of dichloromethane and acetonitrile.

¹H NMR (400 MHz, CDCl₃); δ10.40 (1H, br s), 7.43-7.39 (2H, m), 7.29-7.20 (3H, m), 4.50 (2H, q), 2.99 (2H, q), 1.44 (3H, t), 1.37 (3H, t)

### Preparation Example 5-1-4

### 2-Ethyl-5-ureido-thiazole-4-carboxylic acid ethyl ester

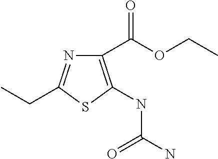

The compound (4.44 g, 13.86 mmol) obtained from Preparation Example 5-1-3 was dissolved in N,N-dimethylformamide 50 mL. Saturated ammonia 40 mL was added thereto and stirred for 12 hours. The reaction mixture was distilled under reduced pressure and solidified in 1:1 mixture of hexane and ethyl acetate. The solid obtained by filtration under reduced pressure was washed with 1:1 mixture of hexane and ethyl acetate and dried to give the title compound (2.85 g, 85%).

¹H NMR (400 MHz, DMSO, d₆); δ 7.15 (2H, br s), 4.31 (2H, q), 2.80 (2H, q), 1.31 (3H, t), 1.24 (3H, t)

### Preparation Example 5-1-5

### 2-Ethyl-4H-thiazolo[5,4-d]pyrimidine-5,7-dione

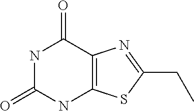

The compound (0.42 g, 1.74 mmol) obtained from Preparation Example 5-1-4 was added in sodium ethoxide solution which was prepared by dissolving sodium (0.08 g, 3.48 mol) in ethanol 15 mL. Formed solid was filtered at room temperature, dried, dissolved in water again and acidified (pH=3) with 6 N hydrochloric aqueous solution. The solid obtained by acidification was filtered, washed and dried to give the title compound (0.24 g, 70%).

¹H NMR (400 MHz, CDCl₃); δ 3.25 (2H, q), 1.51 (3H, t)

### Preparation Example 5-1-6

### 5,7-Dichloro-2-ethyl-thiazolo[5,4-d]pyrimidine

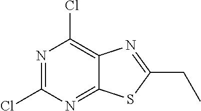

The compound (2.18 g, 11 mmol) obtained from Preparation Example 5-1-5 was suspended in phosphorous oxychloride 15 mL and stirred for 16 hours with reflux. After the reaction mixture was distilled under reduced pressure, the title compound (2.35 g, 91%) was obtained by column-chromatography using 5:1 mixture of hexane and ethyl acetate.

¹H NMR (400 MHz, CDCl₃); δ 3.25 (2H, q), 1.51 (3H, t)

### Preparation Example 5-1-7

### 5-Chloro-2-ethyl-7-(3-trifluoromethyl-5,6-dihydro-8H-[1,2,4]triazolo[4,3-a]pyrazin-7-yl)-thiazolo[5,4-d]pyrimidine

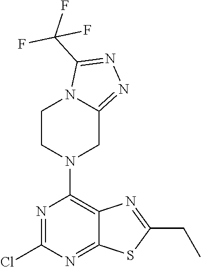

Except for using the compound (234 mg, 1 mmol) obtained from Preparation Example 5-1-6 instead of the compound obtained from Preparation Example 1-1-1, the title compound (333 mg, 85%) was obtained by the same method as that described in Preparation Example 1-1-3.

¹H NMR (400 MHz, CDCl₃); δ 5.90 (2H, br s), 4.77 (2H, br s), 4.35 (2H, t), 3.11 (2H, q), 1.46 (3H, t)

### Example 5-1

### 4-[2-Ethyl-7-(3-trifluoromethyl-5,6-dihydro-8H-[1,2,4]triazolo[4,3-a]pyrazin-7-yl)-thiazolo[5,4-d]pyrimidin-5-yl]-piperazin-2-one

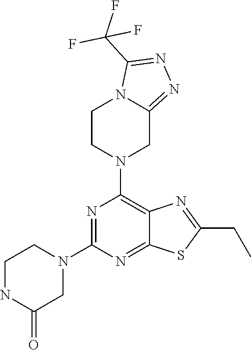

The compound (39 mg, 0.1 mmol) obtained from Preparation Example 5-1-7 and piperazin-2-one (20 mg, 0.2 mmol) was diluted with butanol 2 mL, heated to 150° C. in microwave reactor and stirred for 2 hours. The reaction solution was cooled to room temperature, distilled under reduced pressure, diluted with dichloromethane and washed with water. After the organic layer was dried with anhydrous magnesium sulfate and distilled under reduced pressure, the title compound (39 mg, 80%) was obtained by column-chromatography using 92:8 mixture of dichloromethane and methanol.

¹H NMR (400 MHz, CDCl₃); δ 6.56 (1H, br s), 5.70 (2H, br s), 4.77 (2H, br s), 4.43 (2H, s), 4.32 (2H, t), 4.05 (2H, t), 3.48 (2H, m), 3.01 (2H, q), 1.41 (3H, t)

### Example 5-2

### 2-[2-Ethyl-7-(3-trifluoromethyl-5,6-dihydro-8H-[1,2,4]triazolo[4,3-a]pyrazin-7-yl)-thiazolo[5,4-d]pyrimidin-5-ylamino]-ethanol

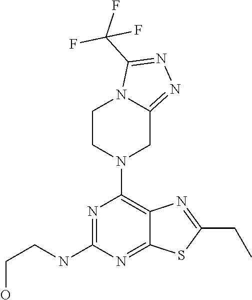

Except for using ethanolamine (12 mg, 0.2 mmol) instead of piperazin-2-one, the title compound (40 mg, 98%) was obtained by the same method as that described in Example 5-1.

¹H NMR (400 MHz, CDCl₃); δ 5.63 (2, br s), 5.44 (1H, t), 4.78 (2H, t), 4.30 (2H, t), 3.83 (2H, t), 3.60 (2H, dd), 2.98 (2H, q), 4.39 (3H, t)

### Example 5-3

### 2-Ethyl-5-(4-methyl-piperazin-1-yl)-7-(3-trifluoromethyl-5,6-dihydro-8H-[1,2,4]triazolo[4,3-a]pyrazin-7-yl)-thiazolo[5,4-d]pyrimidine

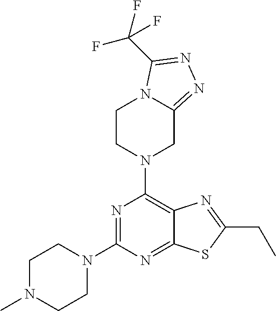

Except for using 1-methyl-piperazine (20 mg, 0.2 mmol) instead of piperazin-2-one, the title compound (44 mg, 98%) was obtained by the same method as that described in Example 5-1.

¹H NMR (400 MHz, CDCl₃); δ5.59 (2H, br s), 4.83 (2H, t), 4.31 (2H, t), 3.83 (4H, t), 2.99 (2H, q), 2.47 (4H, t), 2.35 (3H, s), 1.40 (3H, t)

### Preparation Example 5-4-1

### Butyrylamino-cyano-acetic acid ethyl ester

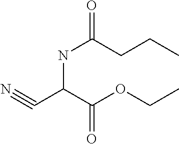

Except for using butyryl chloride (10.96 mL, 106 mmol) instead of propionyl chloride, the title compound (4.6 mg, 16%) was obtained by the same method as that described in Preparation Example 5-1-1.

¹H NMR (400 MHz, CDCl₃); δ 6.18 (1H, d), 5.54 (1H, d), 4.35 (2H, q), 2.28 (2H, t), 1.70 (2H, m), 1.36 (3H, t), 0.98 (3H, t)

### Preparation Example 5-4-2

### 5-Amino-2-propyl-thiazole-4-carboxylic acid ethyl ester

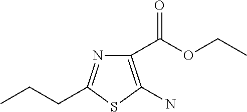

Except for using the compound (4.056 g, 20.46 mmol) obtained from Preparation Example 5-4-1 instead of the compound obtained from Preparation Example 5-1-1, the title compound (2.806 g, 64%) was obtained by the same method as that described in Preparation Example 5-1-2.

¹H NMR (400 MHz, CDCl₃); δ 5.87 (2H, br s), 4.37 (2H, q), 2.61 (2H, t), 1.72 (2H, m), 1.37 (3H, t), 0.97 (3H, t)

### Preparation Example 5-4-3

### 5-Phenoxycarbonylamino-2-propyl-thiazole-4-carboxylic acid ethyl ester

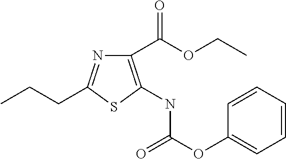

Except for using the compound (777 mg, 3.63 mmol) obtained from Preparation Example 5-4-2 instead of the compound obtained from Preparation Example 5-1-2, the title compound (788 mg, 64%) was obtained by the same method as that described in Preparation Example 5-1-3.

¹H NMR (400 MHz, CDCl₃); δ 10.40 (1H, br s), 7.42-7.39 (2H, m), 7.28-7.20 (3H, m), 4.48 (2H, q), 2.92 (2H, t), 1.78 (2H, m), 1.45 (3H, t), 1.00 (3H, t)

### Preparation Example 5-4-4

### 2-Propyl-5-ureido-thiazole-4-carboxylic acid ethyl ester

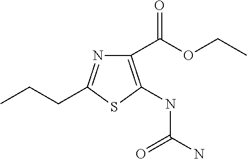

Except for using the compound (788 mg, 2.36 mmol) obtained from Preparation Example 5-4-3 instead of the compound obtained from Preparation Example 5-1-3, the title compound (521 mg, 86%) was obtained by the same method as that described in Preparation Example 5-1-4.

¹H NMR (400 MHz, DMSO, d₆); δ 10.01 (1H, s), 7.15 (2H, br s), 4.32 (2H, q), 2.77 (2H, t), 1.68 (2H, m), 1.33 (3H, t), 0.94 (3H, t)

### Preparation Example 5-4-5

### 2-Propyl-4H-thiazolo[5,4-d]pyrimidine-5,7-dione

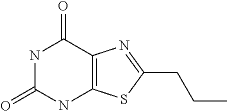

Except for using the compound (363 mg, 1.41 mmol) obtained from Preparation Example 5-4-4 instead of the compound obtained from Preparation Example 5-1-4, the title compound (221 mg, 76%) was obtained by the same method as that described in Preparation Example 5-1-5.

¹H NMR (400 MHz, CDCl₃); δ 11.73 (1H, br s), 11.19 (1H, s), 2.85 (2H, t), 1.67 (2H, m), 0.91 (3H, t)

### Preparation Example 5-4-6

### 5,7-Dichloro-2-propyl-thiazolo[5,4-d]pyrimidine

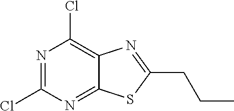

Except for using the compound (410 mg, 1.94 mmol) obtained from Preparation Example 5-4-5 instead of the compound obtained from Preparation Example 5-1-5, the title compound (481 mg, 94%) was obtained by the same method as that described in Preparation Example 5-1-6.

¹H NMR (400N Hz, CDCl₃); δ 3.16 (2H, t), 1.95 (2H, m), 1.09 (3H, t)

### Preparation Example 5-4-7

### 5-Chloro-2-propyl-7-(3-trifluoromethyl-5,6-dihydro-8H-[1,2,4]triazolo[4,3-a]pyrazin-7-yl)-thiazolo[5,4-d]pyrimidine

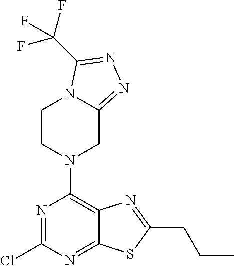

Similar to the method described in Preparation Example 1-1-3, the title compound (0.77 g, 60%) was obtained by using the compound (0.79 g, 3.18 mmol) obtained from Preparation Example 5-4-6 and the compound (0.734 g, 3.82 mmol) obtained from Preparation Example 1-1-2.

¹H NMR (400 MHz, CDCl₃); δ 5.89 (2H, br), 4.77 (2H, br), 4.33 (2H, t), 3.03 (2H, t), 1.84 (2H, m), 1.05 (3H, t)

### Example 5-4

### 4-[2-Propyl-7-(3-trifluoromethyl-5,6-dihydro-8H-[1,2,4]triazolo[4,3-a]pyrazin-7-yl)-thiazolo[5,4-d]pyrimidin-5-yl]-piperazin-2-one

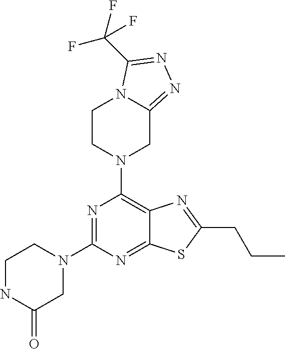

The compound (40 mg, 0.01 mmol) obtained from Preparation Example 5-4-7 and piperazin-2-one (20 mg, 0.2 mmol) was diluted with butanol 2 mL, heated to 150° C. and stirred for 2 hours. The reaction mixture was cooled to room temperature, distilled under reduced pressure, diluted with dichloromethane and washed with water. After the organic layer was dried with anhydrous magnesium sulfate and distilled under reduced pressure, the title compound (37 mg, 79%) was obtained by column-chromatography using 92:8 mixture of dichloromethane and methanol.

¹H NMR (400 MHz, CDCl₃); δ 6.60 (1H, br s), 5.70 (2H, br s), 4.77 (2H, br s), 4.43 (2H, s), 4.31 (2H, t), 4.05 (2H, t), 3.49 (2H, m), 2.95 (2H, t), 1.84 (2H, m), 1.05 (3H, t)

### Example 5-5

### (S)-1-[2-Propyl-7-(3-trifluoromethyl-5,6-dihydro-8H-[1,2,4]triazolo[4,3-a]pyrazin-7-yl)-thiazolo[5,4-d]pyrimidin-5-yl]-pyrrolidin-3-ylamine

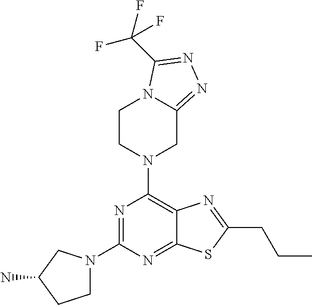

Except for using 3-(S)-Boc-amino pyrrolidine (37 mg, 0.2 mmol) instead of piperazin-2-one, the title compound (28 mg, 62%) was obtained by the same method as that described in Example 5-4.

¹H NMR (400 MHz, CDCl₃); δ 5.61 (2H, m), 4.79 (2H, m), 4.29 (2H, t), 3.88˜3.72 (3H, m), 3.61 (1H, m), 3.42 (1H, dd), 2.91 (2H, t), 2.22 (1H, m), 1.95˜1.76 (3H, m), 1.03 (3H, t)

### Example 5-6

### (S)-1-[2-Propyl-7-(3-trifluoromethyl-5,6-dihydro-8H-[1,2,4]triazolo[4,3-a]pyrazin-7-yl)-thiazolo[5,4-d]pyrimidin-5-yl]-piperidine-3-carboxylic acid ethyl ester

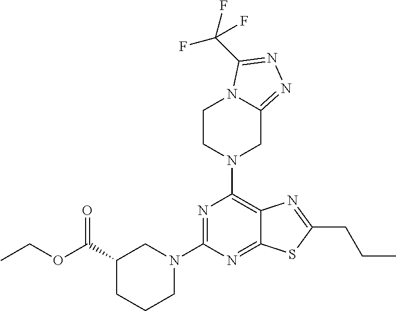

The compound (51 mg, 0.126 mmol) obtained from Preparation Example 5-4-7, piperidine-3-(S)-carboxylic acid ethyl ester (40 mg, 0.252 mmol), palladium acetate (II) (3 mg, 0.013 mmol), BINAP (9 mg, 0.015 mmol) and Cesium carbonate (62 mg, 0.189 mmol) were dissolved in toluene 5 mL and stirred for 5 hour with reflux. The reaction mixture was cooled to room temperature and filtered through Celite. The solvent was removed by distillation under reduced pressure. The title compound (16 mg, 24%) was obtained by column-chromatography using 1:1 mixture of hexane and ethyl acetate.

¹H NMR (400 MHz, CDCl₃); δ 5.62 (2H, m), 4.85˜4.67 (3H, m), 4.48 (1H, m), 4.32 (2H, t), 4.16 (2H, q), 3.24 (1H, dd), 3.07 (1H, m), 2.93 (2H, t), 2.51 (1H, m), 2.08 (1H, m), 1.88˜1.68 (4H, m), 1.54 (1H, m), 1.28 (3H, t), 1.04 (3H, t)

### Example 5-7

### (S)-1-[2-Propyl-7-(3-trifluoromethyl-5,6-dihydro-8H-[1,2,4]triazolo[4,3-a]pyrazin-7-yl)-thiazolo[5,4-d]pyrimidin-5-yl]-piperidine-3-carboxylic acid

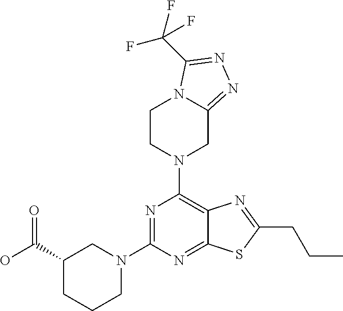

The compound (13 mg, 0.024 mmol) obtained from Example 5-6 was dissolved in tetrahydrofuran 3 mL and methanol 0.5 mL. 1.0 M sodium hydroxide aqueous solution (0.072 mL, 0.072 mmol) was added thereto and stirred for 16 hours. The reaction mixture was acidified with 1.0 M hydrochloric acid aqueous solution and distilled under reduced pressure to remove the solvent. The remaining mixture was diluted with ethyl acetate and washed with water and brine. After the organic layer was dried with anhydrous magnesium sulfate and distilled under reduced pressure, the title compound (9 mg, 75%) was obtained by column-chromatography using 98:2 mixture of dichloromethane and methanol.

¹H NMR (400 MHz, CDCl₃); δ5.62 (2H, m), 4.91˜4.68 (3H, m), 4.49 (1H, d), 4.32 (2H, t), 3.29 (1H, dd), 3.09 (1H, t), 2.92 (2H, t), 2.57 (1H, m), 2.14 (1H, m), 1.89˜1.72 (4H, m), 1.55 (1H, m), 1.04 (3H, t)

### Preparation Example 6-1-1

### 3-Pentafluoroethyl-5,6,7,8-tetrahydro-[1,2,4]triazolo[4,3-a]pyrazine

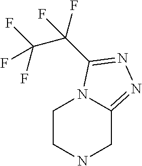

The title compound was synthesized by the method of a following refrerence (See, Journal of Medicinal Chemistry 2005, 48(1), 141-151).

### Preparation Example 6-1-2

### 7-(2-Chloro-6-ethyl-thieno[2,3-d]pyrimidin-4-yl)-3-pentafluoroethyl-5,6,7,8-tetrahydro-[1,2,4]triazolo[4,3-a]pyrazine

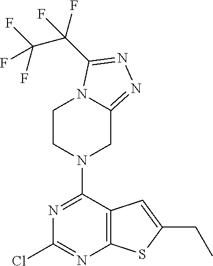

The compound (47 mg, 0.2 mmol) obtained from Preparation Example 2-2-1 and the compound (58 mg, 0.24 mmol) obtained from Preparation Example 6-1-1 were dissolved in N,N-dimethylformamide 5 mL. Diisopropyl amine (65 mg, 0.5 mmol) was added thereto and stirred for 16 hours. After the organic layer was dried with anhydrous magnesium sulfate and distilled under reduced pressure to solidify, the title compound (68 mg, 77%) was obtained by cleanse the solid with diethyl ether.

¹H NMR (400 MHz, CDCl₃); δ 7.05 (1H, s), 5.38 (2H, s), 4.40 (4H, m), 2.95 (2H, q), 1.39 (3H, t)

### Example 6-1

### 4-[6-Ethyl-4-(3-pentafluoroethyl-5,6-dihydro-8H-[1,2,4]triazolo[4,3-a]pyrazin-7-yl)-thieno[2,3-d]pyrimidin-2-yl]-piperazin-2-one

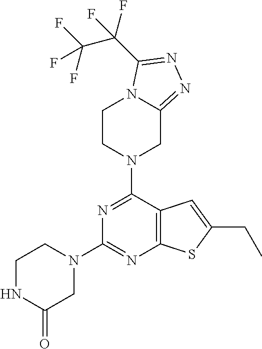

The compound (34 mg, 0.077 mmol) obtained from Preparation Example 6-1-2 and piperazin-2-one (15 mg, 0.154 mmol) was dissolved in butanol 2 mL, heated to 150° C. in microwave reactor and stirred for 2 hours. The reaction solution was cooled to room temperature, distilled under reduced pressure, diluted with dichloromethane and washed with water. After the organic layer was dried with anhydrous magnesium sulfate and distilled under reduced pressure, the title compound (23 mg, 59%) was obtained by column-chromatography using 92:8 mixture of dichloromethane and methanol.

¹H NMR (400 MHz, CDCl₃); δ 6.83 (2H, s), 5.24 (2H, s), 4.41 (2H, s), 4.39 (2H, t), 4.24 (2H, t), 4.05 (2H, t), 3.48 (2H, m), 2.85 (2H, q), 1.35 (3H, t)

### Example 6-2

### 3-[6-Ethyl-4-(3-pentafluoroethyl-5,6-dihydro-8H-[1,2,4]triazolo[4,3-a]pyrazin-7-yl)-thieno[2,3-d]pyrimidin-2-ylamino]-propane-1,2-diol

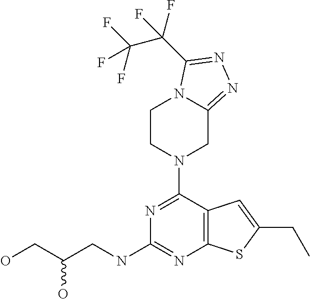

Except for using 3-amino-1,2-propanediol (14 mg, 0.15 mmol) instead of piperazin-2-one, the title compound (26 mg, 70%) was obtained by the same method as that described in Example 6-1.

¹H NMR (400 MHz, CDCl₃); δ 6.82 (1H, s), 5.22 (2H, s), 5.20 (1H, m), 4.36 (2H, t), 4.21 (2H, t), 3.84 (1H, m), 3.68˜3.52 (4H, m), 3.39 (1H, br s), 2.84 (2H, q), 1.34 (31-1,

### Preparation Example 6-3-1

### Acetic Acid

### 2-acetoxy-3-[6-ethyl-4-(3-pentafluoroethyl-5,6-dihydro-8H-[1,2,4]triazolo[4,3-a]pyrazin-7-yl)-thieno[2,3-d]pyrimidin-2-yloxy]-propyl ester

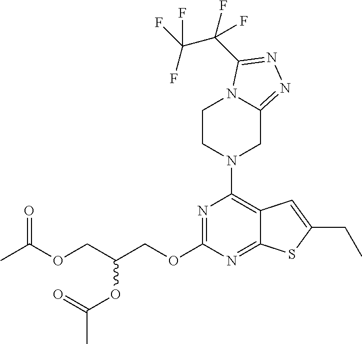

The compound (37 mg, 0.1 mmol) obtained from Preparation Example 3-49-5 and the compound (29 mg, 0.12 mmol) obtained from Preparation Example 6-1-1 were dissolved in butanol 5 mL. Diisopropylethylamine (32 mg, 0.25 mmol) was added thereto, heated to 160° C. and stirred for 16 hours. The reaction mixture was cooled to room temperature, distilled under reduced pressure, diluted with dichloromethane and washed with water. After the organic layer was dried with anhydrous magnesium sulfate and distilled under reduced pressure, the title compound (46 mg, 79%) was obtained by column-chromatography using 1:2 mixture of hexane and ethyl acetate.

¹H NMR (400 MHz, CDCl₃); δ 6.96 (1H, s), 5.43 (1H, m), 5.34 (2H, s), 4.60˜4.26 (8H, m), 2.90 (2H, q), 2.10 (3H, s), 2.09 (3H, s), 1.37 (3H, t)

### Example 6-3

### 3-[6-Ethyl-4-(3-pentafluoroethyl-5,6-dihydro-8H-[1,2,4]triazolo[4,3-a]pyrazin-7-yl)-thieno[2,3-d]pyrimidin-2-yloxy]-propane-1,2-diol

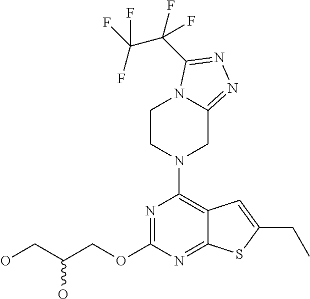

Except for using the compound (46 mg, 0.08 mmol) obtained from Preparation Example 6-3-1 instead of the compound obtained from Preparation Example 3-49-6, the title compound (46 mg, 96%) was obtained by the same method as that described in Example 3-49.

¹H NMR (400 MHz, CDCl₃); δ 6.97 (1H, s), 5.34 (2H, s), 4.50 (2H, m), 4.41 (2H, t), 4.31 (2H, t), 4.12 (1H, m), 3.75 (2H, m), 3.13 (1H, br s), 2.90 (2H, q), 2.33 (1H, br s), 1.37 (3H, t)

### Preparation Example 6-4-1

### 7-(2-Chloro-6-propyl-thieno[2,3-d]pyrimidin-4-yl)-3-pentafluoroethyl-5,6,7,8-tetrahydro-[1,2,4]triazolo[4,3-a]pyrazine

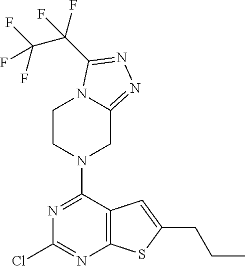

Except for using the compound (49 mg, 0.2 mmol) obtained from Preparation Example 1-1-1 instead of the compound obtained from Preparation Example 2-2-1, the title compound (89 mg, 98%) was obtained by the same method as that described in Preparation Example 6-1-2.

¹H NMR (400 MHz, CDCl₃); δ 7.05 (1H, s), 5.38 (2H, s), 4.40 (4H, m), 2.89 (2H, t), 1.77 (2H, m), 1.02 (3H, t)

### Example 6-4

### 4-[4-(3-Pentafluoroethyl-5,6-dihydro-8H-[1,2,4]triazolo[4,3-a]pyrazin-7-yl)-6-propyl-thieno[2,3-d]pyrimidin-2-yl]-piperazin-2-one

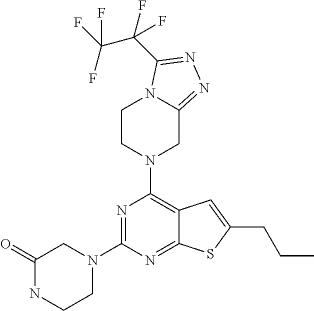

The compound (45 mg, 0.1 mmol) obtained from Preparation Example 6-4-1 and piperazine-2-one (20 mg, 0.2 mmol) were dissolved in butanol 2 mL. The reaction mixture was heated to 150° C. in microwave reactor and stirred for 2 hours. The reaction solution was cooled to room temperature, distilled under reduced pressure, diluted with dichloromethane and washed with water. After the organic layer was dried with anhydrous magnesium sulfate and distilled under reduced pressure, the title compound (34 mg, 65%) was obtained by column-chromatography using 95:5 mixture of dichloromethane and methanol.

¹H NMR (400 MHz, CDCl₃); δ 6.83 (1H, s), 6.70 (1H, s), 5.24 (2H, s), 4.42 (2H, s), 4.39 (2H, t), 4.24 (2H, t), 4.05 (2H, t), 3.48 (1H, m), 2.79 (2H, t), 1.73 (2H, m), 1.00 (3H, t)

### Preparation Example 6-5-1

### 4-[4-(3-Pentafluoroethyl-5,6-dihydro-8H-[1,2,4]triazolo[4,3-a]pyrazin-7-yl)-6-propyl-thieno[2,3-d]pyrimidin-2-yl]-piperazine-1-carboxylic acid tert-butyl ester

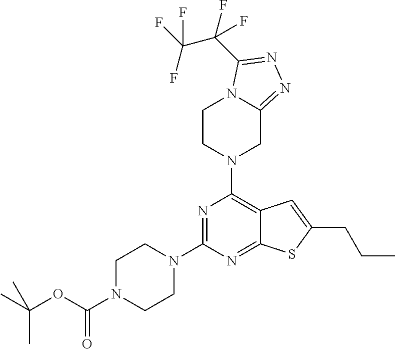

Except for using piperazine-1-carboxylic acid t-butyl ester (37 mg, 0.2 mmol) instead of piperazin-2-one, the title compound (40 mg, 67%) was obtained by the same method as that described in Example 6-4.

¹H NMR (400 MHz, CDCl₃); δ 6.79 (1H, s), 5.19 (2H, s), 4.38 (2H, t), 4.19 (2H, t), 3.79 (4H, t), 3.49 (4H, t), 2.78 (2H, t), 1.72 (2H, m), 1.49 (9H, s), 1.00 (3H, t)

### Example 6-5

### 3-Pentafluoroethyl-7-(2-piperazin-1-yl-6-propyl-thieno[2,3-d]pyrimidin-4-yl)-5,6,7,8-tetrahydro-[1,2,4]triazolo[4,3-a]pyrazine

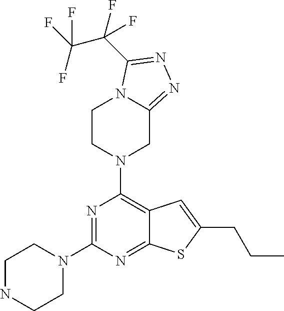

30 mg (79%) of the title compound was obtained by the same method of Example 1-4 except that 40 mg (0.066 mmol) of the compound obtained from Preparation Example 6-5-1 was used instead of the compound obtained from Preparation Example 1-4-1.

¹H NMR (400 MHz, DMSO, d₆); δ 9.01 (2H, br s), 7.28 (1H, s), 5.17 (2H, s), 4.39 (2H, t), 4.23 (2H, t), 3.94 (4H, t), 3.15 (4H, br s), 2.79 (2H, t), 1.66 (2H, m), 0.95 (3H, t)

### Example 6-6

### 7-[2-(4-Methyl-piperazin-1-yl)-6-propyl-thieno[2,3-d]pyrimidin-4-yl]-3-pentafluoroethyl-5,6,7,8-tetrahydro-[1,2,4]triazolo[4,3-a]pyrazine

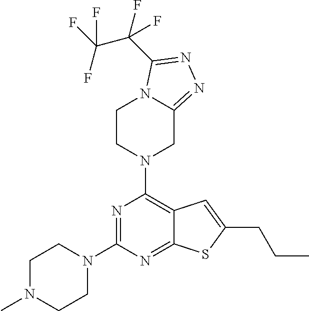

43 mg (93%) of the title compound was obtained by the same method of Example 6-4 except that 18 mg (0.2 mmol) of 1-methyl-piperazin was used instead of piperazin-2-one.

¹H NMR (400 MHz, CDCl₃); δ 6.77 (1H, s), 5.18 (2H, s), 4.38 (2H, t), 4.18 (2H, t), 3.82 t), 2.78 (2H, t), 2.47 (4H, t), 2.34 (3H, s), 1.72 (2H, m), 0.99 (3H, t)

### Example 6-7

### 2-[4-(3-Pentafluoroethyl-5,6-dihydro-8H-[1,2,4]triazolo[4,3-a]pyrazin-7-yl)-6-propyl-thieno[2,3-d]pyrimidin-2-ylamino]-ethanol

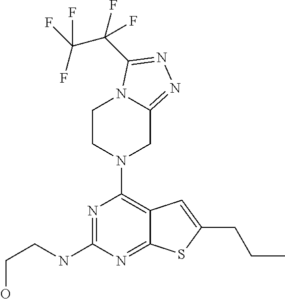

35 mg (81%) of the title compound was obtained by the same method of Example 6-4 except that 11 mg (0.2 mmol) of ethanolamine was used instead of piperazin-2-one.

¹H NMR (400 MHz, CDCl₃); δ 6.80 (1H, s), 5.37 (1H, t), 5.19 (2H, s), 4.36 (2H, t), 4.19 (2H, t), 3.82 (2H, t), 3.60 (2H dd), 2.77 (2H, t), 1.71 (2H, m), 0.99 (3H, t)

### Preparation Example 6-8-1

### 7-[2-(2,2-Dimethyl-[1,3]dioxolan-4-ylmethoxy)-6-propyl-thieno[2,3-d]pyrimidin-4-yl]-3-pentafluoroethyl-5,6,7,8-tetrahydro-[1,2,4]triazolo[4,3-a]pyrazine

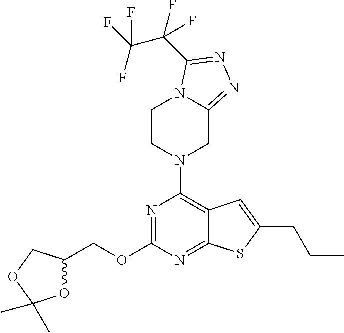

45 mg (0.1 mmol) of the compound obtained from Preparation Example 6-4-1, 26 mg (0.2 mmol) of (2,2-dimethyl-[1,3]dioxolan-4-yl)-methanol, 2.2 mg (0.01 mmol) of palladiumacetate(II), 7.5 mg (0.012 mmol) of BINAP and 49 mg (0.15 mmol) of cesium carbonate were diluted in 3 ml of toluene, and performed reflux with stirring for 3 hours. The reaction solution was cooled to room temperature and filtrated by using sellaite followed by removing the solvent by vacuum distillation. It was purified by column chromatography using a mixture of hexane and ethylacetate with the ratio of 3:2 to obtain 28 mg (51%) of the title compound.

¹H NMR (400 MHz, CDCl₃); δ 6.95 (1H, s), 5.33 (2H, s), 4.57˜4.27 (7H, m), 4.17 (1H, dd), 3.91 (1H, dd), 2.83 (2H, t), 1.75 (2H, m), 1.47 (3H, s), 1.39 (3H, s), 1.01 (3H, t)

### Example 6-8

### 3-[4-(3-Pentafluoroethyl-5,6-dihydro-8H-[1,2,4]triazolo[4,3-a]pyrazin-7-yl)-6-propyl-thieno[2,3-d]pyrimidin-2-yloxy]-propane-1,2-diol

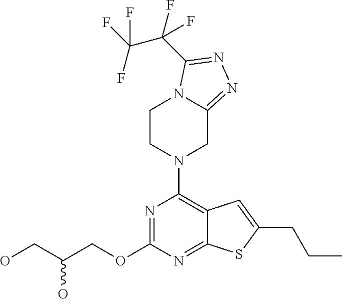

22 mg (85%) of the title compound was obtained by the same method of Example 3-1 except that 28 mg (0.051 mmol) of the compound obtained from Preparation Example 6-8-1 was used instead of the compound obtained from Preparation Example 3-1-1.

¹H NMR (400 MHz, CDCl₃); δ 6.95 (1H, s), 5.33 (2H, s), 4.48 (2H, m), 4.41 (2H, t), 4.32 (2H, t), 4.12 (1H, m), 3.77 (2H, m), 2.83 (2H, t), 1.74 (2H, m), 1.01 (3H, t)

### Preparation Example 6-9-1

### 7-{2-[2-(tert-Butyl-dimethyl-silanyloxy)-ethoxy]-6-propyl-thieno[2,3-d]pyrimidin-4-yl}-3-pentafluoroethyl-5,6,7,8-tetrahydro-[1,2,4]triazolo[4,3-a]pyrazine

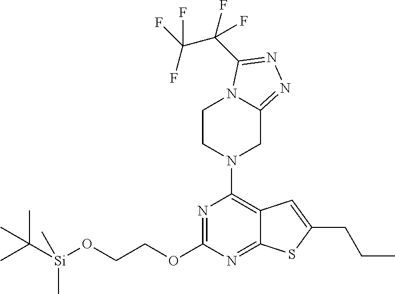

35 mg (59%) of the title compound was obtained by the same method of Preparation Example 6-8-1 except that 35 mg (0.2 mmol) of the compound obtained from Preparation Example 3-3-1 was used instead of (2,2-dimethyl-[1,3]dioxolan-4-yl)-methanol.

¹H NMR (500 MHz, CDCl₃); δ 6.86 (1H, s), 5.23 (2H, s), 4.36 (2H, t), 4.32 (2H, t), 4.21 (2H, t), 3.90 (2H, t), 2.74 (2H, t), 1.66 (2H, m), 0.96 (3H, t), 0.80 (9H, s), 0.01 (6H, s)

### Example 6-9

### 2-[4-(3-Pentafluoroethyl-5,6-dihydro-8H-[1,2,4]triazolo[4,3-a]pyrazin-7-yl)-6-propyl-thieno[2,3-d]pyrimidin-2-yloxy]-ethanol

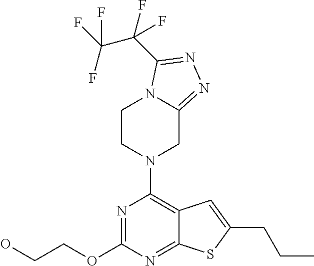

27 mg (96%) of the title compound was obtained by the same method of Example 3-3 except that 35 mg (0.059 mmol) of the compound obtained from Preparation Example 6-9-1 was used instead of the compound obtained from Preparation Example 3-3-2.

¹H NMR (500 MHz, CDCl₃); δ6.96 (1H, s), 5.33 (2H, s), 4.52 (2H, dd), 4.41 (2H, t), 4.32 (2H, t), 3.99 (2H, t), 2.83 (2H, t), 2.77 (1H, br s), 1.75 (2H, m), 1.01 (3H, t)

### Preparation Example 6-10-1

### 7-(2-Chloro-6-ethyl-thieno[2,3-d]pyrimidin-4-yl)-5,6,7,8-tetrahydro-[1,2,4]triazolo[4,3-a]pyrazine

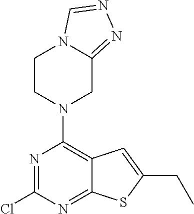

52 mg (81%) of the title compound was obtained by the same method of Preparation Example 6-1-2 except that 30 mg (0.24 mmol) of the compound obtained from Preparation Example 1-75-1 was used instead of the compound obtained from Preparation Example 6-1-1.

¹H NMR (400 MHz, CDCl₃); δ 8.25 (1H, s), 7.12 (1H, s), 5.38 (2H, s), 4.38 (4H, m), 2.98 (2H, q), 1.43 (3H, t)

### Example 6-10

### 4-[4-(5,6-Dihydro-8H-[1,2,4]triazolo[4,3-a]pyrazin-7-yl)-6-ethyl-thieno[2,3-d]pyrimidin-2-yl]-piperazin-2-one

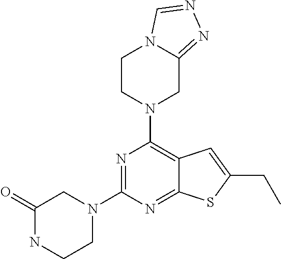

35 mg (56%) of the title compound was obtained by the same method of Example 6-1 except that 52 mg (0.162 mmol) of the compound obtained from Preparation Example 6-10-1 was used instead of the compound obtained from Preparation Example 6-1-2.

¹H NMR (400 MHz, CDCl₃); δ 8.18 (1H, s), 6.87 (2H, s), 6.21 (1H, br s), 5.21 (2H, s), 4.43 (2H, s), 4.25 (4H, m), 4.05 (2H, t), 3.48 (2H, m), 2.85 (2H, q), 1.34 (3H, t)

### Preparation Example 6-11-1

### Acetic Acid

### 2-acetoxy-3-[4-(5,6-dihydro-8H-[1,2,4]triazolo[4,3-a]pyrazin-7-yl)-6-ethyl-thieno[2,3-d]pyrimidin-2-yloxy]-propyl ester

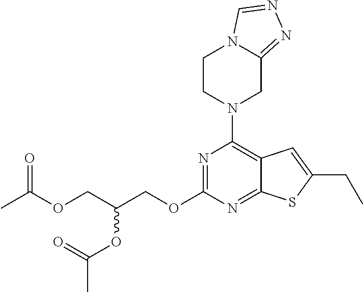

28 mg (61%) of the title compound was obtained by the same method of Preparation Example 6-3-1 except that 15 mg (0.12 mmol) of the compound obtained from Preparation Example 1-75-1 was used instead of the compound obtained from Preparation Example 6-1-1.

¹H NMR (400 MHz, CDCl₃); δ 8.19 (1H, s), 6.99 (1H, s), 5.43 (1H, m), 5.29 (2H, s), 4.60˜4.24 (8H, m), 2.89 (2H, q), 2.10 (3H, s), 2.08 (3H, s), 1.36 (3H, t)

### Example 6-11

### 3-[4-(5,6-Dihydro-8H-[1,2,4]triazolo[4,3-a]pyrazin-7-yl)-6-ethyl-thieno[2,3-d]pyrimidin-2-yloxy]-propane-1,2-diol

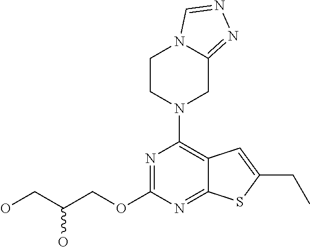

22 mg (96%) of the title compound was obtained by the same method of Example 3-49 except that 28 mg (0.061 mmol) of the compound obtained from Preparation Example 6-11-1 was used instead of the compound obtained from Preparation Example 3-49-6.

¹H NMR (400 MHz, CD₃OD); δ 8.53 (1H, s), 7.24 (1H, s), 5.27 (2H, s), 4.49 (1H, dd), 4.45˜4.30 (5H, m), 4.02 (1H, m), 3.75 (2H, m), 3.13 (1H, br s), 2.94 (2H, q), 1.38 (3H, t)

### Preparation Example 6-12-1

### 3-Methyl-5,6,7,8-tetrahydro-[1,2,4]triazolo[4,3-a]pyrazine

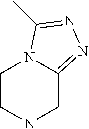

It was synthesized by the method of known patent (reference: WO 03/004498).

### Preparation Example 6-12-2

### 7-(2-Chloro-6-ethyl-thieno[2,3-d]pyrimidin-4-yl)-3-methyl-5,6,7,8-tetrahydro-[1,2,4]triazolo[4,3-a]pyrazine

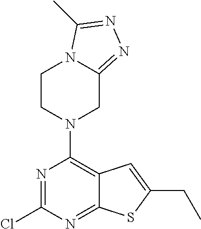

59 mg (88%) of the title compound was obtained by the same method of Preparation Example 6-1-2 except that 33 mg (0.24 mmol) of the compound obtained from Preparation Example 6-12-1 was used instead of the compound obtained from Preparation Example 6-1-1.

¹H NMR (400 MHz, CDCl₃); δ 7.06 (1H, s), 5.28 (2H, s), 4.34 (2H, t), 4.10 (2H, t), 2.93 (2H, q), 2.46 (3H, s), 1.38 (3H, t)

### Example 6-12

### 4-[6-Ethyl-4-(3-methyl-5,6-dihydro-8H-[1,2,4]triazolo[4,3-a]pyrazin-7-yl)-thieno[2,3-d]pyrimidin-2-yl]-piperazin-2-one

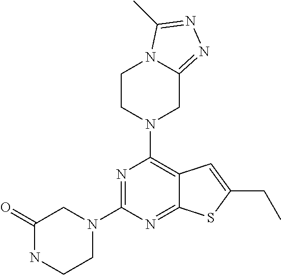

50 mg (71%) of the title compound was obtained by the same method of Example 6-1 except that 59 mg (0.176 mmol) of the compound obtained from Preparation Example 6-12-2 was used instead of the compound obtained from Preparation Example 6-1-2.

¹H NMR (400 MHz, CDCl₃); δ 6.87 (1H, s), 5.13 (2H, s), 4.40 (2H, s), 4.22 (2H, t), 4.08 (2H, t), 4.03 (2H, t), 3.46 (2H, t), 2.85 (2H, q), 1.34 (3H, t)

### Preparation Example 6-13-1

### Acetic Acid

### 2-acetoxy-3-[6-ethyl-4-(3-methyl-5,6-dihydro-8H-[1,2,4]triazolo[4,3-a]pyrazin-7-yl)-thieno[2,3-d]pyrimidin-2-yloxy]-propyl ester

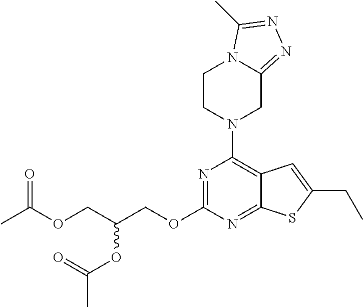

30 mg (64%) of the title compound was obtained by the same method of Preparation Example 6-3-1 except that 17 mg (0.12 mmol) of the compound obtained from Preparation Example 6-12-1 was used instead of the compound obtained from Preparation Example 6-1-1.

¹H NMR (400 MHz, CDCl₃); δ6.98 (1H, s), 5.43 (1H, m), 5.24 (2H, s), 4.53 (2H, m), 4.45 (1H, dd), 4.34˜4.26 (3H, m), 4.09 (2H, t), 2.88 (2H, q), 2.45 (3H, s), 2.10 (3H, s), 2.08 (3H, s), 1.36 (3H, t)

### Example 6-13

### 3-[6-Ethyl-4-(3-methyl-5,6-dihydro-8H-[1,2,4]triazolo[4,3-a]pyrazin-7-yl)-thieno[2,3-d]pyrimidin-2-yloxy]-propane-1,2-diol

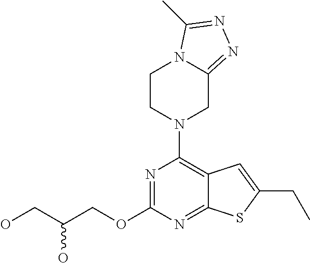

24 mg (96%) of the title compound was obtained by the same method of Example 3-49 except that 30 mg (0.063 mmol) of the compound obtained from Preparation Example 6-13-1 was used instead of the compound obtained from Preparation Example 3-49-6.

¹H NMR (400 MHz, CD₃OD); δ 6.99 (1H, s), 5.24 (2H, s), 4.50 (1H, m), 4.31 (2H, t), 4.09 (1H, m), 4.08 (2H, t), 3.73 (1H, m), 3.27 (1H, d), 2.89 (2H, q), 2.46 (1H, d), 2.44 (3H, s), 1.36 (3H, t)

### Preparation Example 6-14-1

### 7-(2-Chloro-6-propyl-thieno[2,3-d]pyrimidin-4-yl)-3-methyl-5,6,7,8-tetrahydro-[1,2,4]triazolo[4,3-a]pyrazine

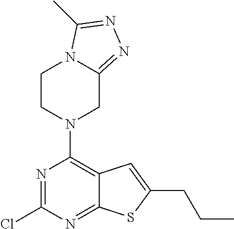

186 mg (87%) of the title compound was obtained by the same method of Preparation Example 1-1-3 except that 100 mg (0.72 mmol) of the compound obtained from Preparation Example 6-12-1 was used instead of the compound obtained from Preparation Example 1-1-2.

¹H NMR (500 MHz, CDCl₃); δ 7.06 (1H, s), 5.31 (2H, s), 4.40 (2H, t), 4.20 (2H, t), 2.87 (2H, t), 2.55 (3H, s), 1.76 (2H, m), 1.01 (3H, t)

### Example 6-14

### 4-[4-(3-Methyl-5,6-dihydro-8H-[1,2,4]triazolo[4,3-a]pyrazin-7-yl)-6-propyl-thieno[2,3-d]pyrimidin-2-yl]-piperazin-2-one

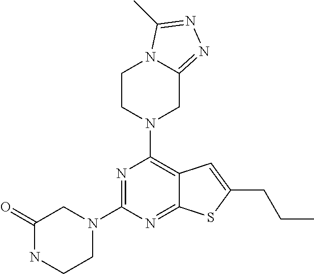

40 mg (0.115 mmol) of the compound obtained from Preparation Example 6-14-1 and 35 mg (0.35 mmol) of piperazin-2-one were diluted in 2 mL of butanol, and followed by heating to 150° C. and stirring for 2 hours in microwave reactor. The reaction solution was cooled to room temperature and vacuum distillation was performed. It was purified by column chromatography using a mixture of methanol and dichloromethane with the ratio of 5:59. The purified and isolated concentrate was diluted in 2 mL of dichloromethane, and 0.3 mL of 2N hydrochloric acid diethyl ether solution was added followed by stirring for 1 hour. The solvent was removed by vacuum distillation to be solid and washed with ether. 40 mg (85%) of the title compound was obtained.

¹H NMR (500 MHz, DMSO, d₆); δ 8.01 (1H, br s), 7.20 (1H, s), 5.15 (2H, s), 4.40 (2H, t), 4.20 (2H, t), 4.08 (2H, t), 4.03 (2H, t), 3.46 (2H, t), 2.75 (2H, t), 2.50 (3H, s), 1.65 (2H, m), 0.90 (3H, t)

### Preparation Example 6-15-1

### 7-[2-(2,2-Dimethyl-[1,3]dioxolan-4-ylmethoxy)-6-propyl-thieno[2,3-d]pyrimidin-4-yl]-3-methyl-5,6,7,8-tetrahydro-[1,2,4]triazolo[4,3-a]pyrazine

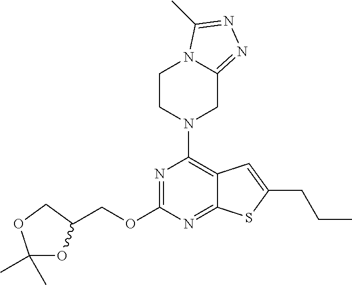

38 mg (75%) of the title compound was obtained by the same method of Preparation Example 6-8-1 except that 40 mg (0.11 mmol) of the compound obtained from Preparation Example 6-14-1 was used instead of the compound obtained from Preparation Example 6-4-1.

Mass: M+H 445

### Example 6-15

### 3-[4-(3-Methyl-5,6-dihydro-8H-[1,2,4]triazolo[4,3-a]pyrazin-7-yl)-6-propyl-thieno[2,3-d]pyrimidin-2-yloxy]-propane-1,2-diol

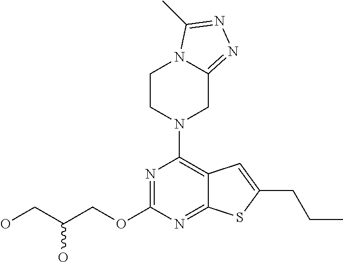

30 mg (79%) of the title compound was obtained by the same method of Example 3-1 except that 41 mg (0.092 mmol) of the compound obtained from Preparation Example 6-15-1 was used instead of the compound obtained from Preparation Example 3-1-1.

¹H NMR (400 MHz, CDCl₃); δ 6.95 (1H, s), 5.18 (2H, s), 4.47 (2H, m), 4.28 (2H, t), 4.12 (1H, m), 4.07 (2H, t), 3.76 (2H, m), 2.80 (2H, t), 2.42 (3H, s), 1.72 (2H, m), 1.00 (3H, t)

### Preparation Example 6-16-1

### 7-{2-[2-(tert-Butyl-dimethyl-silanyloxy)-ethoxy]-6-propyl-thieno[2,3-d]pyrimidin-4-yl}-3-methyl-5,6,7,8-tetrahydro-[1,2,4]triazolo[4,3-a]pyrazine

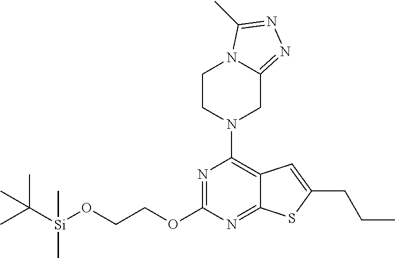

40 mg (0.12 mmol) of the compound obtained from Preparation Example 6-14-1, 40 mg (0.22 mmol) of the compound obtained from Preparation Example 3-3-1, 2 mg (0.009 mmol) of palladiumacetate(II), 7 mg (0.011 mmol) of BINAP and 56 mg (0.17 mmol) of cesium carbonate were diluted in 5 mL of toluene, and performed reflux with stirring for 3 hours. The reaction solution was cooled to room temperature and filtrated by using sellaite followed by removing the solvent by vacuum distillation. It was purified by column chromatography using a mixture of methanol and dichloromethane with the ratio of 10:90 to obtain 36 mg (64%) of the title compound.

¹H NMR (500 MHz, CDCl₃); δ 6.95 (1H, s), 5.21 (2H, s), 4.43 (2H, t), 4.27 (2H, t), 4.08 (2H, t), 3.97 (2H, t) 2.80 (2H, t), 2.44 (3H, s), 1.74 (2H, m), 0.99 (3H, t), 0.88 (9H, s), 0.06 (6H, s)

### Example 6-16

### 2-[4-(3-Methyl-5,6-dihydro-8H-[1,2,4]triazolo[4,3-a]pyrazin-7-yl)-6-propyl-thieno[2,3-d]pyrimidin-2-yloxy]-ethanol

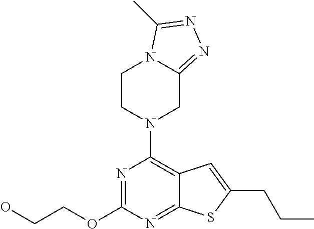

36 mg (0.074 mmol) of the compound obtained from Preparation Example 6-16-1 was dissolved in 5 mL of tetrahydropuran and added 0.37 mL (0.37 mmol) of 1M tetrabutylammonium fluoride followed by stirring for 1 hour. 20 mL of ethylacetate was added and the reaction solution was washed twice with 10 mL of water. The organic layer was dried with magnesium sulfate anhydrous and performed vacuum distillation. 15 mg (56%) of the title compound was obtained by column chromatography using a mixture of methanol and dichloromethane with the ratio of 7.5:92.5.

¹H NMR (500 MHz, CDCl₃); δ 6.97 (1H, s), 5.23 (2H, s), 4.51 (2H, t), 4.30 (2H, m), 4.11 (2H, m), 3.97 (2H, t), 2.81 (2H, t), 2.46 (3H, s), 1.72 (2H, m), 1.00 (3H, t)

### Preparation Example 6-17-1

### 7-{2-[3-(tert-Butyl-dimethyl-silanyloxy)-propoxy]-6-propyl-thieno[2,3-d]pyrimidin-4-yl}-3-methyl-5,6,7,8-tetrahydro-[1,2,4]triazolo[4,3-a]pyrazine

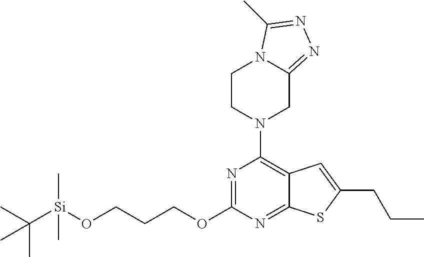

79 mg (78%) of the title compound was obtained by the same method of Preparation Example 6-16-1 except that 63 mg (0.43 mmol) of the compound obtained from Preparation Example 3-10-1 was used instead of the compound obtained from Preparation Example 3-3-1.

¹H NMR (500 MHz, CDCl₃); δ6.94 (1H, s), 5.30 (2H, s), 4.44 (2H, t), 4.39 (2H, t), 4.26 (2H, t), 3.80 (2H, t), 2.83 (2H, t), 2.66 (3H, s), 2.01 (2H, m), 1.74 (2H, m), 1.00 (3H, t), 0.88 (9H, s), 0.04 (6H, s)

### Example 6-17

### 3-[4-(3-Methyl-5,6-dihydro-8H-[1,2,4]triazolo[4,3-a]pyrazin-7-yl)-6-propyl-thieno[2,3-d]pyrimidin-2-yloxy]-propan-1-ol

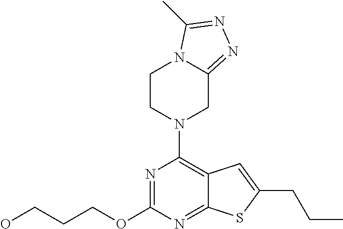

40 mg (66%) of the title compound was obtained by the same method of Example 6-16 except that 79 mg (0.16 mmol) of the compound obtained from Preparation Example 6-17-1 was used instead of the compound obtained from Preparation Example 6-16-1.

¹H NMR (400 MHz, CDCl₃); δ 7.00 (1H, s), 5.29 (2H, s), 4.58 (2H, t), 4.37 (2H, t), 4.17 (2H, t), 3.81 (2H, t), 2.85 (2H, t), 2.55 (3H, s), 2.06 (2H, m), 1.76 (2H, m), 1.04 (3H, t)

### Preparation Example 6-18-1

### 7-[2-(2,2-Dimethyl-[1,3]dioxan-5-ylmethoxy)-6-propyl-thieno[2,3-d]pyrimidin-4-yl]-3-methyl-5,6,7,8-tetrahydro-[1,2,4]triazolo[4,3-a]pyrazine

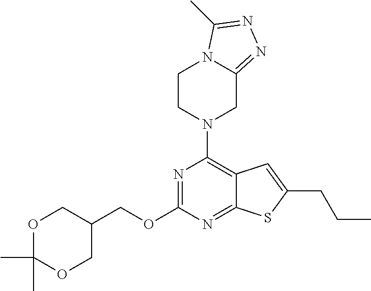

68 mg (69%) of the title compound was obtained by the same method of Preparation Example 6-16-1 except that 73 mg (0.38 mmol) of the compound obtained from Preparation Example 3-2-1 was used instead of the compound obtained from Preparation Example 3-3-1.

¹H NMR (500 MHz, CDCl₃); δ 6.95 (1H, s), 5.24 (2H, s), 4.44 (2H, d), 4.32 (2H, t), 4.14 (2H, t), 4.08 (2H, dd), 3.87 (2H, dd), 2.81 (2H, t), 2.50 (3H, s), 2.22 (1H, m), 1.74 (2H, m), 1.39 (6H, s), 1.00 (3H, t)

### Example 6-18

### 2-[4-(3-Methyl-5,6-dihydro-8H-[1,2,4]triazolo[4,3-a]pyrazin-7-yl)-6-propyl-thieno[2,3-d]pyrimidin-2-yloxymethyl]-propane-1,3-diol

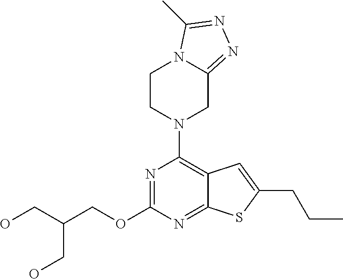

43 mg (69%) of the title compound was obtained by the same method of Example 3-1 except that 68 mg (0.15 mmol) of the compound obtained from Preparation Example 6-18-1 was used instead of the compound obtained from Preparation Example 3-1-1.

¹H NMR (500 MHz, DMSO, d₆); δ 7.33 (1H, s), 5.05 (2H, s), 4.49 (2H, t), 4.24 (2H, d), 4.19 (2H, t), 4.05 (2H, t), 3.48 (4H, m), 2.80 (2H, t), 2.29 (3H, s), 1.94 (1H, m), 1.64 (2H, m), 0.92 (3H, t)

### Preparation Example 6-19-1

### 3-Phenyl-5,6,7,8-tetrahydro-[1,2,4]triazolo[4,3-a]pyrazine

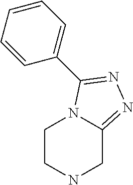

It was synthesized by the method of known publication (reference: Organic Letters 2005, 7(6), 1039-1042).

### Preparation Example 6-19-2

### 7-(2-Chloro-6-ethyl-thieno[2,3-d]pyrimidin-4-yl)-3-phenyl-5,6,7,8-tetrahydro-[1,2,4]triazolo[4,3-a]pyrazine

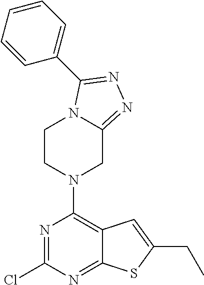

78 mg (99%) of the title compound was obtained by the same method of Preparation Example 6-1-2 except that 48 mg (0.24 mmol) of the compound obtained from Preparation Example 6-19-1 was used instead of the compound obtained from Preparation Example 6-1-1.

¹H NMR (400 MHz, CDCl₃); δ 7.74 (2H, m), 7.54 (3H, m), 7.12 (1H, s), 5.43 (2H, s), 4.34 (4H, s), 2.95 (2H, q), 1.40 (3H, t)

### Example 6-19

### 4-[6-Ethyl-4-(3-phenyl-5,6-dihydro-8H-[1,2,4]triazolo[4,3-a]pyrazin-7-yl)-thieno[2,3-d]pyrimidin-2-yl]-piperazin-2-one

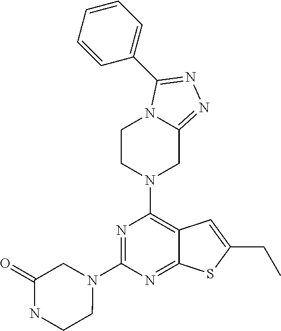

69 mg (75%) of the title compound was obtained by the same method of Example 6-1 except that 79 mg (0.2 mmol) of the compound obtained from Preparation Example 6-19-2 was used instead of the compound obtained from Preparation Example 6-1-2.

¹H NMR (400 MHz, CDCl₃); δ 7.73 (2H, m), 7.52 (3H, m), 6.90 (1H, s), 6.67 (1H, br s), 5.30 (2H, s), 4.43 (2H, s), 4.30 (2H, t), 4.21 (2H, t), 4.06 (2H, t), 3.49 (2H, m), 2.85 (2H, q), 1.35 (3H, t)

### Preparation Example 6-20-1

### 2-Chloro-4-(5,6-dihydro-8H-imidazo[1,2-a]pyrazin-7-yl)-6-ethyl-thieno[2,3-d]pyrimidine

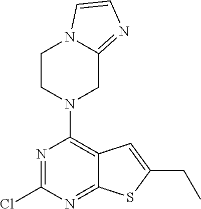

54 mg (84%) of the title compound was obtained by the same method of Preparation Example 6-1-2 except that 30 mg (0.24 mmol) of the compound obtained from Preparation Example 1-77-1 was used instead of the compound obtained from Preparation Example 6-1-1.

¹H NMR (400 MHz, CDCl₃); δ 7.11 (1H, s), 7.07 (1H, s), 6.92 (1H, s), 5.20 (2H, s), 4.36 (2H, t), 4.21 (2H, t), 2.91 (2H, q), 1.37 (3H, t)

### Example 6-20

### 4-[4-(5,6-Dihydro-8H-imidazo[1,2-a]pyrazin-7-yl)-6-ethyl-thieno[2,3-d]pyrimidin-2-yl]-piperazin-2-one

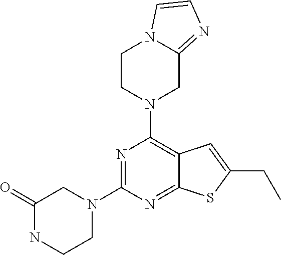

47 mg (69%) of the title compound was obtained by the same method of Example 6-1 except that 54 mg (0.169 mmol) of the compound obtained from Preparation Example 6-20-1 was used instead of the compound obtained from Preparation Example 6-1-2.

¹H NMR (400 MHz, CDCl₃); δ 7.04 (1H, s), 6.90 (1H, s), 6.89 (1H, s), 6.82 (1H, br s), 5.08 (2H, s), 4.43 (2H, s), 4.24 (2H, t), 4.18 (2H, t), 4.04 (2H, t), 3.47 (2H, m), 2.83 (2H, q), 1.33 (3H, t)

### Preparation Example 6-21-1

### Acetic Acid

### 2-acetoxy-3-[4-(5,6-dihydro-8H-imidazo[1,2-a]pyrazin-7-yl)-6-ethyl-thieno[2,3-d]pyrimidin-2-yloxy]-propyl ester

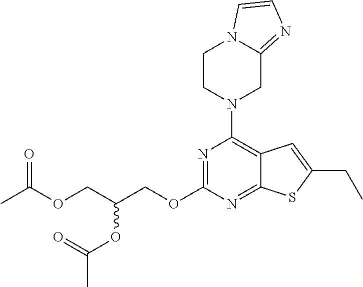

20 mg (43%) of the title compound was obtained by the same method of Preparation Example 6-3-1 except that 15 mg (0.12 mmol) of the compound obtained from Preparation Example 1-77-1 was used instead of the compound obtained from Preparation Example 6-1-1.

¹H NMR (400 MHz, CDCl₃); δ 7.06 (1H, s), 7.02 (1H, s), 6.91 (1H, s), 5.43 (1H, m), 5.16 (2H, s), 4.60˜4.26 (6H, m), 4.20 (2H, t), 2.87 (2H, q), 2.10 (3H, s), 2.08 (3H, s), 1.35 (3H, t)

### Example 6-21

### 3-[4-(5,6-Dihydro-8H-imidazo[1,2-a]pyrazin-7-yl)-6-ethyl-thieno[2,3-d]pyrimidin-2-yloxy]-propane-1,2-diol

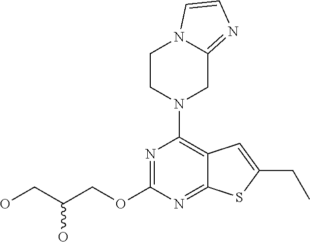

13 mg (76%) of the title compound was obtained by the same method of Example 3-49 except that 20 mg (0.044 mmol) of the compound obtained from Preparation Example 6-21-1 was used instead of the compound obtained from Preparation Example 3-49-6.

¹H NMR (400 MHz, CD₃OD); δ 7.22 (1H, s), 7.12 (1H, s), 7.01 (1H, s), 5.11 (2H, s), 4.48 (1H, dd), 4.40˜4.24 (5H, m), 4.02 (2H, m), 3.75˜3.62 (2H, m), 2.92 (2H, q), 1.37 (3H, t)

### Preparation Example 6-22-1

### 2-Chloro-6-ethyl-4-(2-trifluoromethyl-5,6-dihydro-8H-imidazo[1,2-a]pyrazin-7-yl)-thieno[2,3-d]pyrimidine

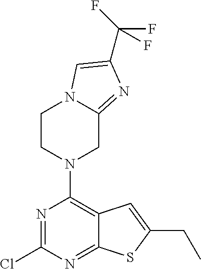

77 mg (99%) of the title compound was obtained by the same method of Preparation Example 6-1-2 except that 46 mg (0.24 mmol) of the compound obtained from Preparation Example 1-78-1 was used instead of the compound obtained from Preparation Example 6-1-1.

¹H NMR (400 MHz, CDCl₃); δ 7.27 (1H, s), 7.06 (1H, s), 5.22 (2H, s), 4.38 (2H, t), 4.26 (2H, t), 2.91 (2H, q), 1.37 (3H, t)

### Example 6-22

### 4-[6-Ethyl-4-(2-trifluoromethyl-5,6-dihydro-8H-imidazo[1,2-a]pyrazin-7-yl)-thieno[2,3-d]pyrimidin-2-yl]-piperazin-2-one

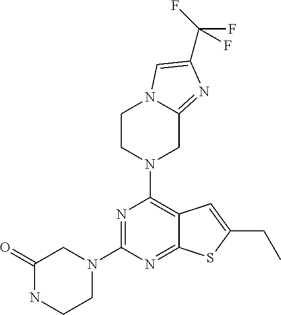

27 mg (77%) of the title compound was obtained by the same method of Example 6-1 except that 30 mg (0.077 mmol) of the compound obtained from Preparation Example 6-22-1 was used instead of the compound obtained from Preparation Example 6-1-2.

¹H NMR (400 MHz, CDCl₃); δ7.25 (1H, s), 6.86 (1H, s), 6.74 (1H, br s), 5.10 (2H, s), 4.42 (2H, s), 4.24 (4H, m), 4.04 (2H, t), 3.47 (2H, m), 2.82 (2H, q), 1.33 (3H, t)

### Preparation Example 6-23-1

### Acetic Acid

### 2-acetoxy-3-[6-ethyl-4-(2-trifluoromethyl-5,6-dihydro-8H-imidazo[1,2-a]pyrazin-7-yl)-thieno[2,3-d]pyrimidin-2-yloxy]-propyl ester

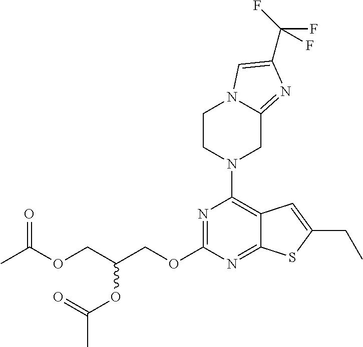

21 mg (75%) of the title compound was obtained by the same method of Preparation Example 6-3-1 except that 12 mg (0.065 mmol) of the compound obtained from Preparation Example 1-78-1 was used instead of the compound obtained from Preparation Example 6-1-1.

¹H NMR (400 MHz, CDCl₃); δ 7.27 (1H, s), 6.80 (1H, s), 5.43 (1H, m), 5.18 (2H, s), 4.60˜4.22 (8H, m), 2.87 (2H, q), 2.10 (3H, s), 2.08 (3H, s), 1.29 (3H, t)

### Example 6-23

### 3-[6-Ethyl-4-(2-trifluoromethyl-5,6-dihydro-8H-imidazo[1,2-a]pyrazin-7-yl)-thieno[2,3-d]pyrimidin-2-yloxy]-propane-1,2-diol

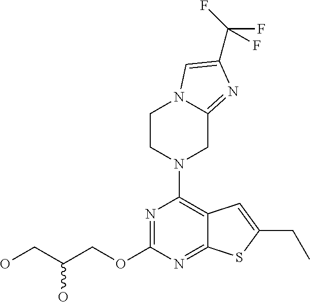

17 mg (94%) of the title compound was obtained by the same method of Example 3-49 except that 21 mg (0.04 mmol) of the compound obtained from Preparation Example 6-23-1 was used instead of the compound obtained from Preparation Example 3-49-6.

¹H NMR (400 MHz, CDCl₃); δ 7.21 (1H, s), 6.92 (1H, s), 5.11 (2H, s), 4.43 (2H, m), 4.28 (2H, t), 4.19 (2H, t), 4.08 (1H, m), 3.78˜3.64 (3H, m), 3.00 (1H, br s), 2.81 (2H, q), 1.29 (3H, t)

### Preparation Example 6-24-1

### 2-Chloro-6-ethyl-4-(1,4,6,7-tetrahydro-pyrazolo[4,3-c]pyridin-5-yl)-thieno[2,3-d]pyrimidine

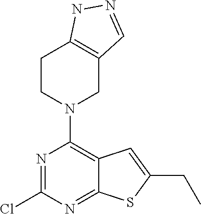

50 mg (78%) of the title compound was obtained by the same method of Preparation Example 6-1-2 except that 47 mg (0.24 mmol) of the compound obtained from Preparation Example 1-76-1 was used instead of the compound obtained from Preparation Example 6-1-1.

¹H NMR (400 MHz, CDCl₃); δ 7.43 (1H, m), 7.02 (1H, s), 4.93 (2H, s), 4.14 (2H, s), 3.10˜2.85 (4H, m), 1.37 (3H, t)

### Example 6-24

### 4-[6-Ethyl-4-(1,4,6,7-tetrahydro-pyrazolo[4,3-c]pyridin-5-yl)-thieno[2,3-d]pyrimidin-2-yl]-piperazin-2-one

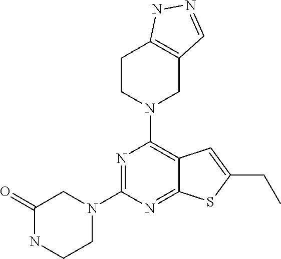

27 mg (45%) of the title compound was obtained by the same method of Example 6-1 except that 50 mg (0.156 mmol) of the compound obtained from Preparation Example 6-24-1 was used instead of the compound obtained from Preparation Example 6-1-2.

¹H NMR (400 MHz, CDCl₃); δ7.4 (1H, s), 6.86 (1H, s), 6.65 (1H, br s), 4.83 (2H, s), 4.47 (2H, s), 4.05 (4H, m), 3.48 (2H, m), 3.01 (2H, t), 2.84 (2H, q), 1.33 (3H, t)

### Preparation Example 6-25-1

### Acetic Acid

### 2-acetoxy-3-[6-ethyl-4-(1,4,6,7-tetrahydro-pyrazolo[4,3-c]pyridin-5-yl)-thieno[2,3-d]pyrimidin-2-yloxy]-propyl ester

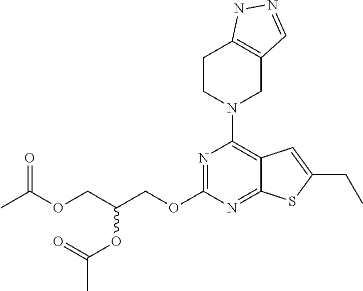

20 mg (43%) of the title compound was obtained by the same method of Preparation Example 6-3-1 except that 24 mg (0.12 mmol) of the compound obtained from Preparation Example 1-76-1 was used instead of the compound obtained from Preparation Example 6-1-1.

¹H NMR (400 MHz, CDCl₃); δ 7.44 (1H, s), 6.95 (1H, s), 5.43 (1H, m), 4.90 (2H, s), 4.63˜4.42 (3H, m), 4.30 (1H, dd), 4.12 (2H, t), 3.03 (2H, t), 2.88 (2H, q), 2.09 (3H, s), 2.08 (3H, s), 1.35 (3H, t)

### Example 6-25

### 3-[6-Ethyl-4-(1,4,6,7-tetrahydro-pyrazolo[4,3-c]pyridin-5-yl)-thieno[2,3-d]pyrimidin-2-yloxy]-propane-1,2-diol

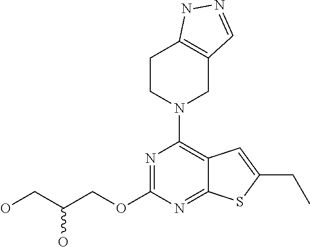

15 mg (88%) of the title compound was obtained by the same method of Example 3-49 except that 20 mg (0.044 mmol) of the compound obtained from Preparation Example 6-25-1 was used instead of the compound obtained from Preparation Example 3-49-6.

¹H NMR (400 MHz, CD₃OD); δ 7.50 (1H, s), 7.20 (1H, s), 4.96 (2H, s), 4.47 (1H, dd), 4.37 (1H, dd), 4.19 (2H, t), 4.02 (1H, m), 3.69 (2H, m), 2.99 (2H, t), 2.92 (2H, q), 1.37 (3H, t)

### Preparation Example 6-26-1

### 3-Trifluoromethyl-4,5,6,7-tetrahydro-1H-pyrazolo[4,3-c]pyridine; hydrochloride

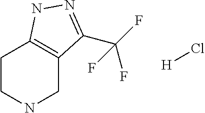

It was synthesized by the method of known patent (reference: WO 2004/064778).

### Preparation Example 6-26-2

### 2-Chloro-6-ethyl-4-(3-trifluoromethyl-1,4,6,7-tetrahydro-pyrazolo[4,3-c]pyridin-5-yl)-thieno[2,3-d]pyrimidine

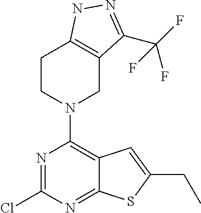

77 mg (99%) of the title compound was obtained by the same method of Preparation Example 6-1-2 except that 55 mg (0.24 mmol) of the compound obtained from Preparation Example 6-26-1 was used instead of the compound obtained from Preparation Example 6-1-1.

¹H NMR (400 MHz, CDCl₃); δ 10.30 (1H, br s), 7.00 (1H, s), 4.97 (2H, s), 4.18 (2H, t) 3.04 (2H, t), 2.93 (2H, q), 1.38 (3H, t)

### Example 6-26

### 4-[6-Ethyl-4-(3-trifluoromethyl-1,4,6,7-tetrahydro-pyrazolo[4,3-c]pyridin-5-yl)-thieno[2,3-d]pyrimidin-2-yl]-piperazin-2-one

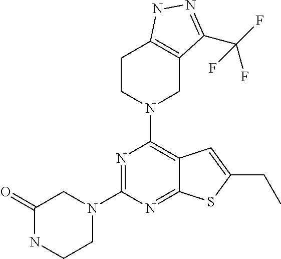

31 mg (69%) of the title compound was obtained by the same method of Example 6-1 except that 39 mg (0.1 mmol) of the compound obtained from Preparation Example 6-26-2 was used instead of the compound obtained from Preparation Example 6-1-2.

¹H NMR (400 MHz, CDCl₃); δ 11.37 (1H, br s), 6.84 (1H, s), 6.54 (1H, s), 4.88 (2H, s), 4.45 (2H, s), 4.05 (4H, m), 3.47 (1H, m), 2.99 (2H, t), 2.84 (2H, q), 1.33 (3H, t)

### Preparation Example 6-27-1

### 2-(2-Chloro-6-ethyl-thieno[2,3-d]pyrimidin-4-yl)-1,2,3,4-tetrahydro-isoquinoline

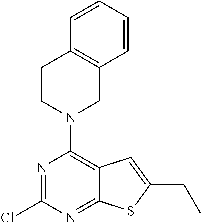

65 mg (98%) of the title compound was obtained by the same method of Preparation Example 6-1-2 except that 32 mg (0.24 mmol) of 1,2,3,4-tetrahydro-isoquinoline was used instead of the compound obtained from Preparation Example 6-1-1.

¹H NMR (400 MHz, CDCl₃); δ 7.30˜7.18 (4H, m), 7.09 (1H, s), 4.98 (2H, s), 4.09 (2H, t), 3.05 (2H, t), 2.90 (2H, q), 1.36 (3H, t)

### Example 6-27

### 4-[4-(3,4-Dihydro-1H-isoquinolin-2-yl)-6-ethyl-thieno[2,3-d]pyrimidin-2-yl]-piperazin-2-one

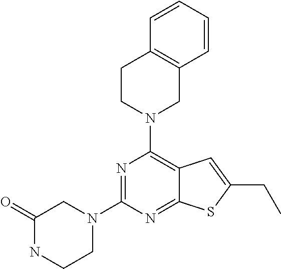

15 mg (63%) of the title compound was obtained by the same method of Example 6-1 except that 20 mg (0.06 mmol) of the compound obtained from Preparation Example 6-27-1 was used instead of the compound obtained from Preparation Example 6-1-2.

¹H NMR (400 MHz, CDCl₃); δ 7.26˜7.14 (4H, m), 6.93 (1H, s), 6.46 (1H, br s), 4.92 (2H, s), 4.49 (2H, s), 4.04 (4H, m), 3.47 (2H, m), 3.03 (2H, t), 2.83 (2H, q), 1.33 (3H, t)

### Preparation Example 6-28-1

### 2-(2-Chloro-6-ethyl-thieno[2,3-d]pyrimidin-4-yl)-6,7-dimethoxy-1,2,3,4-tetrahydro-isoquinoline

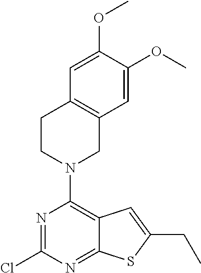

77 mg (99%) of the title compound was obtained by the same method of Preparation Example 6-1-2 except that 55 mg (0.24 mmol) of 6,7-dimethoxy-1,2,3,4-tetrahydro-isoquinoline was used instead of the compound obtained from Preparation Example 6-1-1.

¹H NMR (400 MHz, CDCl₃); δ 7.08 (1H, s), 6.73 (1H, s), 6.70 (1H, s), 4.93 (2H, s), 4.08 (2H, t), 3.88 (6H, s), 2.98 (2H, t), 2.92 (2H, q), 1.26 (3H, t)

### Example 6-28

### 4-[4-(6,7-Dimethoxy-3,4-dihydro-1H-isoquinolin-2-yl)-6-ethyl-thieno[2,3-d]pyrimidin-2-yl]-piperazin-2-one

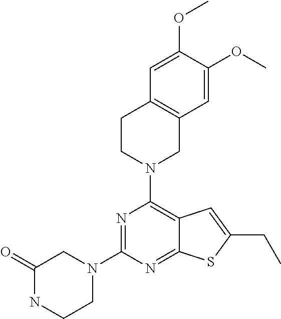

34 mg (76%) of the title compound was obtained by the same method of Example 6-1 except that 39 mg (0.1 mmol) of the compound obtained from Preparation Example 6-28-1 was used instead of the compound obtained from Preparation Example 6-1-2.

¹H NMR (400 MHz, CDCl₃); δ 6.92 (1H, s), 6.72 (1H, s), 6.68 (1H, s), 6.59 (1H, br s), 4.84 (2H, s), 4.49 (2H, s), 4.06 (2H, t), 4.00 (2H, t), 3.90 (3H, s), 3.88 (2H, t), 3.48 (2H, m), 2.95 (2H, t), 2.84 (2H, q), 1.33 (3H, t)

### Preparation Example 6-29-1

### 2-(2-Chloro-6-ethyl-thieno[2,3-d]pyrimidin-4-yl)-2,3,4,9-tetrahydro-1H-beta-carboline

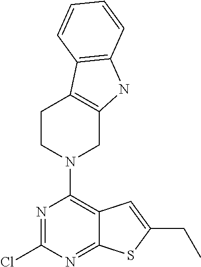

72 mg (97%) of the title compound was obtained by the same method of Preparation Example 6-1-2 except that 41 mg (0.24 mmol) of 2,3,4,9-tetrahydro-1H-beta-carboline was used instead of the compound obtained from Preparation Example 6-1-1.

¹H NMR (400 MHz, CDCl₃); δ 7.87 (1H, s), 7.52 (1H, d), 7.36 (1H, d), 7.20 (1H, t), 7.14 (1H, t), 7.07 (1H, s), 5.05 (2H, s), 4.20 (2H, t), 3.07 (2H, t), 2.94 (2H, q), 1.39 (3H, t)

### Example 6-29

### 4-[6-Ethyl-4-(1,3,4,9-tetrahydro-beta-carbolin-2-yl)-thieno[2,3-d]pyrimidin-2-yl]-piperazin-2-one

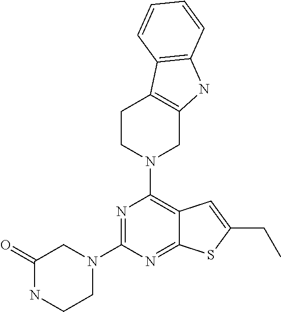

34 mg (40%) of the title compound was obtained by the same method of Example 6-1 except that 72 mg (0.195 mmol) of the compound obtained from Preparation Example 6-29-1 was used instead of the compound obtained from Preparation Example 6-1-2.

¹H NMR (400 MHz, CDCl₃); δ 8.18 (1H, s), 7.51 (1H, d), 7.36 (1H, d), 7.17 (1H, t), 7.12 (1H, t), 6.89 (1H, s), 6.55 (1H, s), 4.07 (2H, t), 4.04 (2H, t), 3.46 (2H, m), 2.84 (2H, q), 1.34 (3H, t)

### Preparation Example 6-30-1

### 4-Trifluoromethyl-5,6,7,8-tetrahydro-pyrido[3,4-d]pyrimidine; hydrochloride

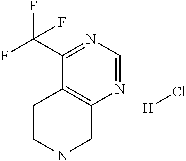

It was synthesized by the method of known patent (reference: WO 2006/104356).

### Preparation Example 6-30-2

### 7-(2-Chloro-6-ethyl-thieno[2,3-d]pyrimidin-4-yl)-4-trifluoromethyl-5,6,7,8-tetrahydro-pyrido[3,4-d]pyrimidine

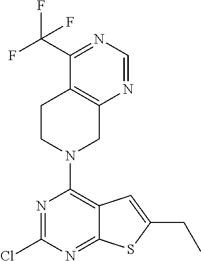

21 mg (30%) of the title compound was obtained by the same method of Preparation Example 6-1-2 except that 51 mg (0.214 mmol) of the compound obtained from Preparation Example 6-30-1 was used instead of the compound obtained from Preparation Example 6-1-1.

¹H NMR (400 MHz, CDCl₃); δ 9.21 (1H, s), 7.13 (1H, s), 5.23 (2H, s), 4.25 (2H, t), 3.30 (2H, t), 2.98 (2H, q), 1.43 (3H, t)

### Example 6-30

### 4-[6-Ethyl-4-(4-trifluoromethyl-5,8-dihydro-6H-pyrido[3,4-d]pyrimidin-7-yl)-thieno[2,3-d]pyrimidin-2-yl]-piperazin-2-one

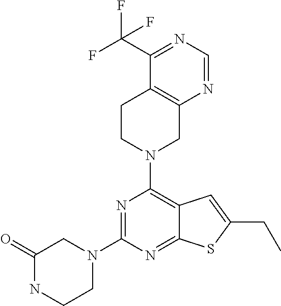

6 mg (24%) of the title compound was obtained by the same method of Example 6-1 except that 21 mg (0.053 mmol) of the compound obtained from Preparation Example 6-30-2 was used instead of the compound obtained from Preparation Example 6-1-2.

¹H NMR (400 MHz, CDCl₃); δ 9.08 (1H, s), 6.83 (1H, s), 6.27 (1H, br s), 5.01 (2H, s), 4.39 (2H, s), 4.03 (2H, t), 3.99 (2H, t), 3.42 (2H, m), 3.16 (2H, t), 2.78 (2H, q), 1.27 (3H, t)

### Preparation Example 6-31-1

### 2,4-Bis-trifluoromethyl-5,6,7,8-tetrahydro-pyrido[3,4-d]pyrimidine; hydrochloride

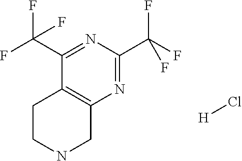

It was synthesized by the method of known patent (reference: WO 2006/104356).

### Preparation Example 6-31-2

### 7-(2-Chloro-6-ethyl-thieno[2,3-d]pyrimidin-4-yl)-2,4-bis-trifluoromethyl-5,6,7,8-tetrahydro-pyrido[3,4-d]pyrimidine

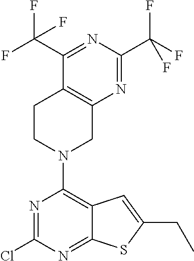

70 mg (74%) of the title compound was obtained by the same method of Preparation Example 6-1-2 except that 74 mg (0.24 mmol) of the compound obtained from Preparation Example 6-31-1 was used instead of the compound obtained from Preparation Example 6-1-1.

¹H NMR (400 MHz, CDCl₃); δ 7.09 (1H, s), 5.27 (2H, s), 4.23 (2H, t), 3.35 (2H, t), 2.96 (2H, q), 1.40 (3H, t)

### Example 6-31

### 4-[4-(2,4-Bis-trifluoromethyl-5,8-dihydro-6H-pyrido[3,4-d]pyrimidin-7-yl)-6-ethyl-thieno[2,3-d]pyrimidin-2-yl]-piperazin-2-one

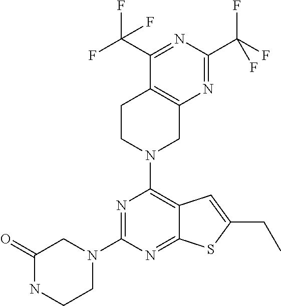

21 mg (57%) of the title compound was obtained by the same method of Example 6-1 except that 33 mg (0.07 mmol) of the compound obtained from Preparation Example 6-31-2 was used instead of the compound obtained from Preparation Example 6-1-2.

¹H NMR (400 MHz, CDCl₃); δ 6.88 (1H, s), 6.35 (1H, br s), 5.30 (2H, s), 4.46 (2H, s), 4.12 (2H, t), 4.06 (2H, t), 3.48 (2H, m), 3.31 (2H, t), 2.87 (2H, q), 1.35 (3H, t)

### Preparation Example 6-32-1

### 3-(4-Chloro-6-ethyl-thieno[2,3-d]pyrimidin-2-yloxy)-propane-1,2-diol

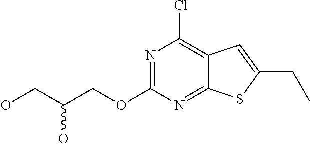

43 mg (55%) of the title compound was obtained by the same method of Example 3-49 except that 101 mg (0.271 mmol) of the compound obtained from Preparation Example 3-49-5 was used instead of the compound obtained from Preparation Example 3-49-6.

¹H NMR (400 MHz, CDCl₃); δ 6.96 (1H, s), 4.51 (2H, m), 4.18 (1H, m), 3.80 (2H, m), 3.20 (1H, br s), 2.91 (2H, q), 2.54 (1H, br s), 1.38 (3H, t)

### Example 6-32

### 3-[4-(2,4-Bis-trifluoromethyl-5,8-dihydro-6H-pyrido[3,4-d]pyrimidin-7-yl)-6-ethyl-thieno[2,3-d]pyrimidin-2-yloxy]-propane-1,2-diol

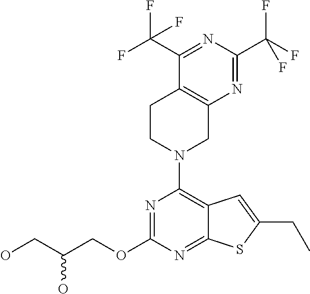

20 mg (0.07 mmol) of the compound obtained from Preparation Example 6-32-1 and 32 mg (0.105 mmol) of the compound obtained from Preparation Example 6-31-1 were dissolved in 2 mL of butanol and 27 mL (0.105 mmol) of diisopropylethylamine was added followed by stirring in microwave reactor for 2 hours at 150° C. The reaction solution was cooled to room temperature and vacuum distillation was performed. It is diluted with dichloromethane and washed with water. The organic layer was dried with magnesium sulfate anhydrous and vacuum distillation was performed. It was purified by column chromatography using a mixture of methanol and dichloromethane with the ratio of 8:92 to obtain 16 mg (43%) of the title compound.

¹H NMR (400 MHz, CDCl₃); δ 7.00 (1H, s), 5.24 (2H, s), 4.51 (2H, m), 4.20 (2H, t), 4.12 (1H, m), 3.76 (2H, m), 3.33 (2H, t), 2.91 (2H, q), 1.37 (3H, t)

### Preparation Example 6-33-1

### 7-(2-Chloro-6-propyl-thieno[2,3-d]pyrimidin-4-yl)-2,4-bis-trifluoromethyl-5,6,7,8-tetrahydro-pyrido[3,4-d]pyrimidine

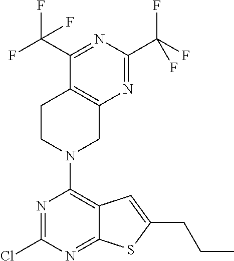

247 mg (1 mmol) of the compound obtained from Preparation Example 1-1-1 and 369 mg (1.2 mmol) of the compound obtained from Preparation Example 6-31-1 were dissolved in 5 mL of N,N-dimethylformamide, and 388 mL (3 mmol) of diisopropylethylamine was added followed by stirring for 16 hours. After performing vacuum distillation of the reaction solution, it was diluted with dichloromethane and washed with water. The organic layer was dried with magnesium sulfate anhydrous and vacuum distillation was performed. It was purified by column chromatography using a mixture of hexane and ethylacetate with the ratio of 5:1 to obtain 367 mg (76%) of the title compound.

¹H NMR (400 MHz, CDCl₃); δ 7.09 (1H, s), 5.27 (2H, s), 4.23 (2H, t), 3.34 (2H, t), 2.89 (2H, t), 1.78 (2H, m), 1.03 (3H, t)

### Example 6-33

### 4-[4-(2,4-Bis-trifluoromethyl-5,8-dihydro-6H-pyrido[3,4-d]pyrimidin-7-yl)-6-propyl-thieno[2,3-d]pyrimidin-2-yl]-piperazin-2-one

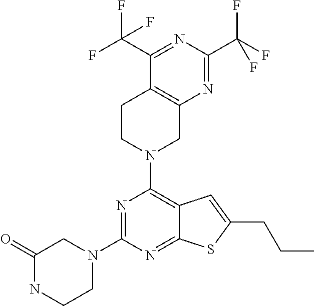

38 mg (69%) of the title compound was obtained by the same method of Example 6-1 except that 48 mg (0.1 mmol) of the compound obtained from Preparation Example 6-33-1 was used instead of the compound obtained from Preparation Example 6-1-2.

¹H NMR (400 MHz, CDCl₃); δ 6.88 (1H, s), 6.72 (1H, br s), 5.15 (2H, s), 4.45 (2H, s), 4.12 (2H, t), 4.05 (21-1H, t), 3.48 (2H, m), 3.31 (2H, t), 2.81 (2H, t), 1.74 (2H, m), 1.01 (3H, t)

### Preparation Example 6-34-1

### 7-(2-Chloro-6-ethyl-thieno[2,3-d]pyrimidin-4-yl)-2-methyl-4-trifluoromethyl-5,6,7,8-tetrahydro-pyrido[3,4-d]pyrimidine

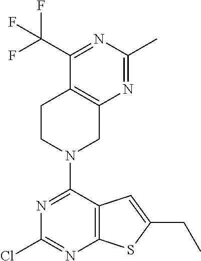

27 mg (57%) of the title compound was obtained by the same method of Preparation Example 6-1-2 except that 34 mg (0.136 mmol) of the compound obtained from Preparation Example 1-80-1 was used instead of the compound obtained from Preparation Example 6-1-1.

¹H NMR (400 MHz, CDCl₃); δ 7.10 (1H, s), 5.13 (2H, s), 4.19 (2H, t), 3.20 (2H, t), 2.93 (2H, q), 2.79 (3H, s), 1.38 (3H, t)

### Example 6-34

### 4-[6-Ethyl-4-(2-methyl-4-trifluoromethyl-5,8-dihydro-6H-pyrido[3,4-d]pyrimidin-7-yl)-thieno[2,3-d]pyrimidin-2-yl]-piperazin-2-one

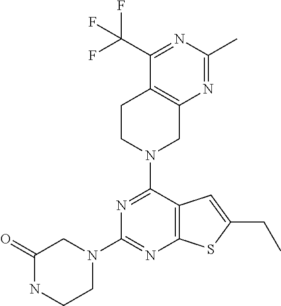

17 mg (55%) of the title compound was obtained by the same method of Example 6-1 except that 27 mg (0.065 mmol) of the compound obtained from Preparation Example 6-34-1 was used instead of the compound obtained from Preparation Example 6-1-2.

¹H NMR (400 MHz, CDCl₃); δ 6.90 (1H, s), 6.42 (1H, br s), 5.02 (2H, s), 4.46 (2H, s), 4.07 (4H, m), 3.48 (2H, m), 3.18 (2H, t), 2.85 (2H, q), 2.78 (3H, s), 1.34 (3H, t)

### Example 6-35

### 3-[6-Ethyl-4-(2-methyl-4-trifluoromethyl-5,8-dihydro-6H-pyrido[3,4-d]pyrimidin-7-yl)-thieno[2,3-d]pyrimidin-2-yloxy]-propane-1,2-diol

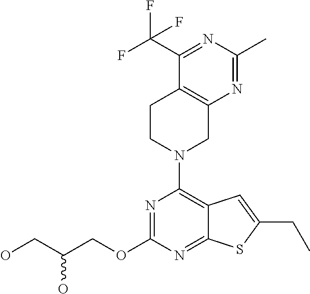

6 mg (21%) of the title compound was obtained by the same method of Example 6-32 except that 15 mg (0.06 mmol) of the compound obtained from Preparation Example 1-80-1 was used instead of the compound obtained from Preparation Example 6-31-1.

¹H NMR (400 MHz, CDCl₃); δ7.03 (1H, s), 5.11 (2H, s), 4.51 (2H, m), 4.15 (2H, t), 4.12 (1H, m), 3.74 (2H, m), 3.42 (1H, br s), 3.19 (2H, t), 2.89 (2H, q), 2.78 (3H, s), 2.57 (1H, br s), 1.36 (3H, t)

### Preparation Example 6-36-1

### 7-(2-Chloro-6-propyl-thieno[2,3-d]pyrimidin-4-yl)-2-methyl-4-trifluoromethyl-5,6,7,8-tetrahydro-pyrido[3,4-d]pyrimidine

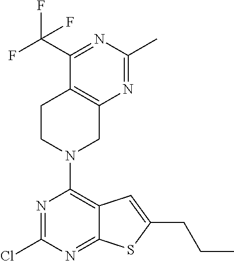

151 mg (88%) of the title compound was obtained by the same method of Preparation Example 6-33-1 except that 122 mg (0.48 mmol) of the compound obtained from Preparation Example 1-80-1 was used instead of the compound obtained from Preparation Example 6-31-1.

¹H NMR (400 MHz, CDCl₃); δ 7.11 (1H, s), 5.18 (2H, s), 4.19 (2H, t), 2.87 (2H, t), 2.79 (3H, s), 1.76 (2H, m), 1.02 (3H, t)

### Example 6-36

### 4-[4-(2-Methyl-4-trifluoromethyl-5,8-dihydro-6H-pyrido[3,4-d]pyrimidin-7-yl)-6-propyl-thieno[2,3-d]pyrimidin-2-yl]-piperazin-2-one

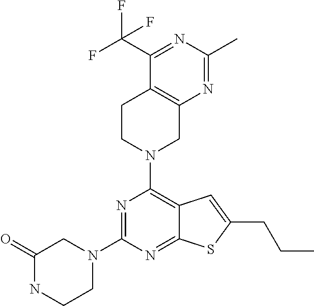

38 mg (78%) of the title compound was obtained by the same method of Example 6-1 except that 43 mg (0.1 mmol) of the compound obtained from Preparation Example 6-36-1 was used instead of the compound obtained from Preparation Example 6-1-2.

¹H NMR (400 MHz, CDCl₃); δ 6.90 (1H, s), 6.50 (1H, s), 5.02 (2H, s), 4.50 (2H, s), 4.07 (2H, q), 3.48 (2H, m), 3.18 (2H, t), 2.77 (2H, t), 2.77 (3H, s), 1.73 (2H, m), 1.00 (3H, t)

### Preparation Example 6-37-1

### 2-Phenyl-4-trifluoromethyl-5,6,7,8-tetrahydro-pyrido[3,4-d]pyrimidine; hydrochloride

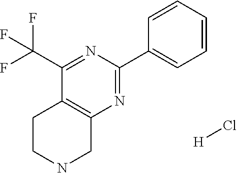

It was synthesized by the method of known patent (reference: WO 2006/104356).

### Preparation Example 6-37-2

### 7-(2-Chloro-6-ethyl-thieno[2,3-d]pyrimidin-4-yl)-2-phenyl-4-trifluoromethyl-5,6,7,8-tetrahydro-pyrido[3,4-d]pyrimidine

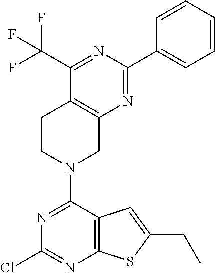

49 mg (64%) of the title compound was obtained by the same method of Preparation Example 6-1-2 except that 61 mg (0.193 mmol) of the compound obtained from Preparation Example 1-37-1 was used instead of the compound obtained from Preparation Example 6-1-1.

¹H NMR (400 MHz, CDCl₃); δ 8.49 (2H, m), 7.51 (3H, m), 7.13 (1H, s), 5.22 (2H, s), 4.22 (2H, t), 3.27 (2H, t), 2.96 (2H, q), 1.04 (3H, t)

### Example 6-37

### 4-[6-Ethyl-4-(2-phenyl-4-trifluoromethyl-5,8-dihydro-6H-pyrido[3,4-d]pyrimidin-7-yl)-thieno[2,3-d]pyrimidin-2-yl]-piperazin-2-one

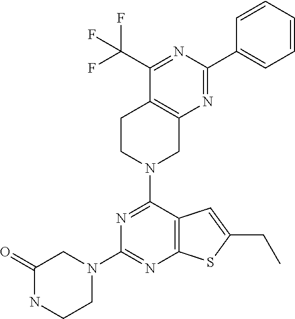

13 mg (45%) of the title compound was obtained by the same method of Example 6-1 except that 25 mg (0.053 mmol) of the compound obtained from Preparation Example 6-37-2 was used instead of the compound obtained from Preparation Example 6-1-2.

¹H NMR (400 MHz, CDCl₃); δ 8.56 (2H, m), 7.56 (3H, m), 6.97 (1H, s), 6.30 (1H, br s), 5.14 (2H, s), 4.55 (2H, s), 4.14 (4H, m), 3.53 (2H, m), 3.28 (2H, t), 2.92 (2H, q), 1.04 (3H, t)

### Preparation Example 6-38-1

### 7-(2-Chloro-6-propyl-thieno[2,3-d]pyrimidin-4-yl)-2-phenyl-4-trifluoromethyl-5,6,7,8-tetrahydro-pyrido[3,4-d]pyrimidine

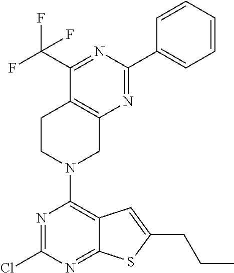

408 mg (83%) of the title compound was obtained by the same method of Preparation Example 6-33-1 except that 379 mg (1.2 mmol) of the compound obtained from Preparation Example 6-37-1 was used instead of the compound obtained from Preparation Example 6-31-1.

¹H NMR (400 MHz, CDCl₃); δ 8.50 (2H, m), 7.52 (3H, m), 7.12 (1H, s), 5.22 (2H, s), 4.22 (2H, t), 3.27 (2H, t), 2.90 (2H, t), 1.79 (2H, m), 1.04 (3H, t)

### Example 6-38

### 4-[4-(2-Phenyl-4-trifluoromethyl-5,8-dihydro-6H-pyrido[3,4-d]pyrimidin-7-yl)-6-propyl-thieno[2,3-d]pyrimidin-2-yl]-piperazin-2-one

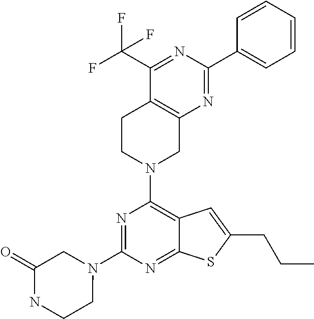

34 mg (62%) of the title compound was obtained by the same method of Example 6-1 except that 49 mg (0.1 mmol) of the compound obtained from Preparation Example 6-38-1 was used instead of the compound obtained from Preparation Example 6-1-2.

¹H NMR (400 MHz, CDCl₃+CD₃OD); δ8.50 (2H, m), 7.52 (3H, m), 6.95 (1H, s), 5.22 (2H, s), 4.22 (2H, t), 3.27 (2H, t), 2.90 (2H, t), 1.79 (2H, m), 1.04 (3H, t)

### Preparation Example 6-39-1

### 2-Furan-3-yl-4-trifluoromethyl-5,6,7,8-tetrahydro-pyrido[3,4-d]pyrimidine; hydrochloride

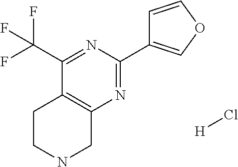

It was synthesized by the method of known patent (reference: WO 2006/104356).

### Preparation Example 6-39-2

### 7-(2-Chloro-6-ethyl-thieno[2,3-d]pyrimidin-4-yl)-2-furan-3-yl-4-trifluoromethyl-5,6,7,8-tetrahydro-pyrido[3,4-d]pyrimidine

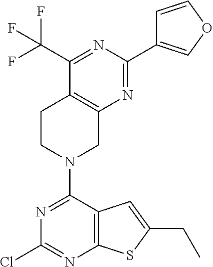

91 mg (98%) of the title compound was obtained by the same method of Preparation Example 6-1-2 except that 73 mg (0.24 mmol) of the compound obtained from Preparation Example 1-39-1 was used instead of the compound obtained from Preparation Example 6-1-1.

¹H NMR (400 MHz, CDCl₃); δ 8.30 (1H, s), 7.52 (1H, s), 7.11 (1H, s), 7.09 (1H, s), 5.15 (2H, s), 4.19 (2H, t), 3.23 (2H, t), 2.96 (2H, q), 1.40 (3H, t)

### Example 6-39

### 4-[6-Ethyl-4-(2-furan-3-yl-4-trifluoromethyl-5,8-dihydro-6H-pyrido[3,4-d]pyrimidin-7-yl)-thieno[2,3-d]pyrimidin-2-yl]-piperazin-2-one

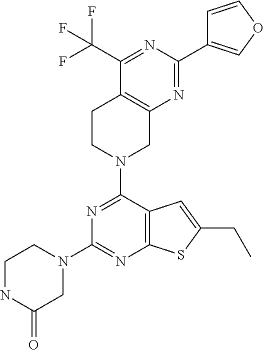

28 mg (0.06 mmol) of the compound obtained from Preparation Example 6-39-2 and 12 mg (0.12 mmol) of piperazine-2-one were diluted with 2 mL of butanol followed by heating to 150° C. and stirring for 2 hours in microwave reactor. The reaction solution was cooled to room temperature and vacuum distillation was performed. It was diluted with dichloromethane and washed with water. The organic layer was dried with magnesium sulfate anhydride and vacuum distillation was performed. It was purified by column chromatography using a mixture of methanol and dichloromethane with the ratio of 8:92 to obtain 12 mg (38%) of the title compound.

¹H NMR (400 MHz, CDCl₃); δ 8.32 (1H, s), 7.51 (1H, s), 7.11 (1H, s), 6.91 (1H, s), 6.28 (1H, br s), 5.02 (2H, s), 4.50 (2H, s), 4.08 (4H, t), 3.49 (2H, m), 3.20 (2H, t), 2.87 (2H, q), 1.36 (3H, t)

### Example 6-40

### 3-[6-Ethyl-4-(2-furan-3-yl-4-trifluoromethyl-5,8-dihydro-6H-pyrido[3,4-d]pyrimidin-7-yl)-thieno[2,3-d]pyrimidin-2-ylamino]-propane-1,2-diol

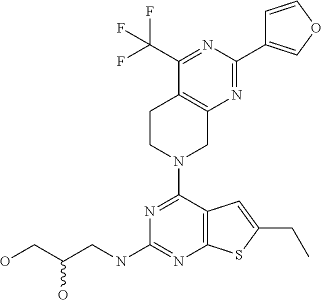

11 mg (35%) of the title compound was obtained by the same method of Example 6-39 except that 11 mg (0.12 mmol) of 3-amino-propane-1,2-diol was used instead of piperazine-2-one.

¹H NMR (400 MHz, CDCl₃); δ 8.29 (1H, s), 7.51 (1H, s), 7.08 (1H, s), 6.90 (1H, s), 5.24 (1H, t), 5.02 (2H, s), 4.07 (4H, t), 3.85 (1H, m), 3.62 (4H, m), 3.17 (2H, t), 2.86 (2H, q), 1.34 (3H, t)

### Preparation Example 6-41-1

### 3-Oxo-piperidine-1,4-dicarboxylic acid 1-tert-butyl ester 4-ethyl ester

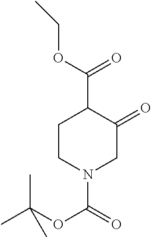

It was synthesized by the method of known publication (reference: J. Med. Chem. 2006, 49, 7843-7853)

### Preparation Example 6-41-2

### 2-Methyl-3-oxo-1,2,3,4,5,7-hexahydro-pyrazolo[3,4-c]pyridine-6-carboxylic acid tert-butyl ester

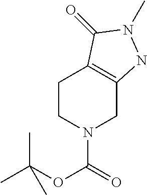

0.37 g (1.36 mmol) of the compound obtained from Preparation Example 6-41-1 was dissolved in 10 mL of toluene followed by adding 94 mg (2.04 mmol) of methylhydrazine and performing reflux with stirring for 16 hours. The reaction solution was cooled to room temperature and the solvent was removed by vacuum distillation. 0.26 g (74%) of the title compound was obtained by purifying with column chromatography using a mixture of methanol and dichloromethane with the ratio of 10:90.

¹H NMR (400 MHz, CD₃OD); δ 4.39 (2H, s), 3.63 (2H, s), 3.48 (3H, s), 2.40 (2H, t), 1.50 (9H, s)

### Preparation Example 6-41-3

### 2-Methyl-1,2,4,5,6,7-hexahydro-pyrazolo[3,4-c]pyridin-3-one; hydrochloride

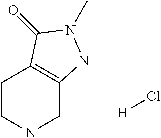

256 mg (1.01 mmol) of the compound obtained from Preparation Example 6-41-2 was dissolved in 10 mL of 4.0M hydrochloric acid dioxane solution followed by stirring for 1 hour. The solvent was removed by vacuum distillation to be a solid and washed with diethyl ether. 190 mg (99%) of the title compound was obtained.

¹H NMR (500 MHz, CD₃OD); δ 4.40 (2H, s), 3.75 (3H, s), 3.51 (2H, t), 2.82 (2H, t)

### Preparation Example 6-41-4

### 6-(2-Chloro-6-ethyl-thieno[2,3-d]pyrimidin-4-yl)-2-methyl-1,2,4,5,6,7-hexahydro-pyrazolo[3,4-c]pyridin-3-one

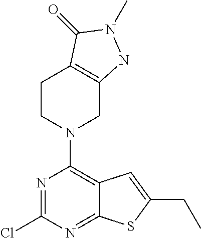

35 mg (0.15 mmol) of the compound obtained from Preparation Example 2-2-1 and 34 mg (0.18 mmol) of the compound obtained from Preparation Example 6-41-3 were diluted in 5 mL of N,N-dimethylformamide and 97 mg (0.75 mmol) of diiospropylethylamine was added followed by stirring for 4 hours. After performing vacuum distillation of the reaction solution, it was diluted with dichloromethane and washed with water. The organic layer was dried with magnesium sulfate anhydrous and vacuum distillation was performed. It was purified by column chromatography using a mixture of hexane and ethylacetate with the ratio of 1:1 to obtain 20 mg (38%) of the title compound.

¹H NMR (400 MHz, CDCl₃); δ 6.97 (1H, s), 4.82 (2H, s), 4.02 (2H, t), 3.59 (3H, s), 2.86 (2H, q), 2.67 (2H, t), 1.32 (3H, t)

### Example 6-41

### 6-[6-Ethyl-2-(3-oxo-piperazin-1-yl)-thieno[2,3-d]pyrimidin-4-yl]-2-methyl-1,2,4,5,6,7-hexahydro-pyrazolo[3,4-c]pyridin-3-one

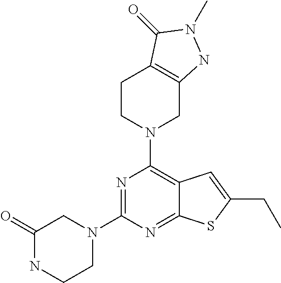

20 mg (0.057 mmol) of the compound obtained from Preparation Example 6-41-4 and 11 mg (0.114 mmol) of piperazine-2-one were diluted in 2 mL of butanol, and followed by heating to 150° C. and stirring for 2 hours in microwave reactor. The reaction solution was cooled to room temperature and vacuum distillation was performed. It was diluted with dichloromethane and washed with water. The organic layer was dried with magnesium sulfate anhydrous and vacuum distillation was performed. It was purified by column chromatography using a mixture of methanol and dichloromethane with the ratio of 15:85 to obtain 7 mg (29%) of the title compound.

¹H NMR (400 MHz, CDCl₃); δ 6.81 (1H, s), 6.72 (1H, br s), 4.67 (2H, s), 4.35 (2H, s), 3.98 (2H, t), 3.88 (2H, t), 3.57 (3H, s), 3.44 (2H, m), 2.80 (2H, q), 2.56 (2H, t), 1.30 (3H, t)

### Preparation Example 6-42-1

### Acetic Acid

### 2-acetoxy-3-[6-ethyl-4-(2-methyl-3-oxo-1,2,3,4,5,7-hexahydro-pyrazolo[3,4-c]pyridin-6-yl)-thieno[2,3-d]pyrimidin-2-yloxy]-propyl ester

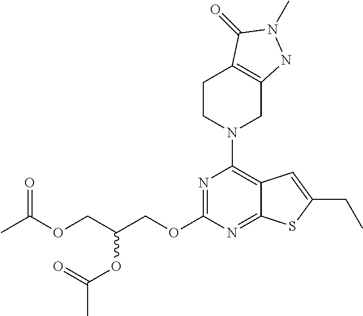

11 mg (37%) of the title compound was obtained by the same method of Preparation Example 6-3-1 except that 14 mg (0.072 mmol) of the compound obtained from Preparation Example 6-41-3 was used instead of the compound obtained from Preparation Example 6-1-1.

¹H NMR (400 MHz, CDCl₃); δ 6.90 (1H, s), 5.43 (1H, m), 4.77 (2H, m), 4.58˜4.42 (3H, m), 4.25 (1H, dd), 3.97 (2H, t), 3.54 (3H, s), 2.86 (2H, q), 2.63 (2H, t), 2.09 (3H, s), 2.08 (3H, s), 1.32 (3H, t)

### Example 6-42

### 6-[2-(2,3-Dihydroxy-propoxy)-6-ethyl-thieno[2,3-d]pyrimidin-4-yl]-2-methyl-1,2,4,5,6,7-hexahydro-pyrazolo[3,4-c]pyridin-3-one

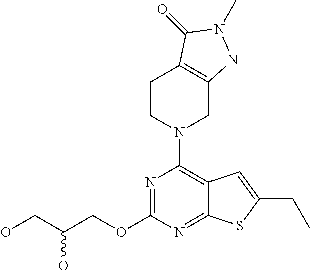

6 mg (67%) of the title compound was obtained by the same method of Example 3-49 except that 11 mg (0.022 mmol) of the compound obtained from Preparation Example 6-42-1 was used instead of the compound obtained from Preparation Example 3-49-6.

¹H NMR (400 MHz, CD₃OD); δ 7.14 (1H, s), 4.83 (2H, s), 4.46 (1H, dd), 4.35 (1H, dd), 4.08 (2H, t), 4.01 (2H, t), 3.69 (2H, m), 3.51 (3H, s), 2.92 (2H, q), 2.63 (2H, t), 1.36 (3H, t)

### Preparation Example 6-43-1

### 2-(2-Hydroxy-ethyl)-3-oxo-1,2,3,4,5,7-hexahydro-pyrazolo[3,4-c]pyridine-6-carboxylic acid tert-butyl ester

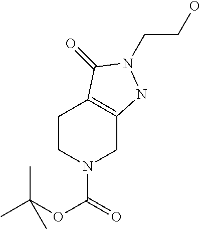

297 mg (75%) of the title compound was obtained by the same method of Preparation Example 6-41-2 except that 160 mg (2.1 mmol) of 2-hydrazino-ethanol was used instead of methyl-hydrazine.

¹H NMR (500 MHz, CD₃OD); δ 4.35 (2H, s), 3.98 (2H, br s), 3.90 (2H, m), 3.58 (2H, t), 2.40 (2H, br s), 1.46 (9H, s)

### Preparation Example 6-43-2

### 2-(2-Hydroxy-ethyl)-1,2,4,5,6,7-hexahydro-pyrazolo[3,4-c]pyridin-3-one; hydrochloride

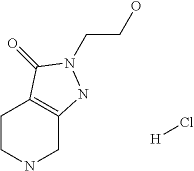

297 mg (75%) of the title compound was obtained by the same method of Preparation Example 6-41-3 except that 297 mg (1.05 mmol) of the compound obtained from Preparation Example 6-43-1 was used instead of the compound obtained from Preparation Example 6-41-2.

¹H NMR (400 MHz, CD₃OD); δ4.39 (2H, s), 4.19 (2H, t), 3.84 (2H, t), 3.51 (2H, t), 2.82 (2H, t)

### Preparation Example 6-43-3

### 6-(2-Chloro-6-ethyl-thieno[2,3-d]pyrimidin-4-yl)-2-(2-hydroxy-ethyl)-1,2,4,5,6,7-hexahydro-pyrazolo[3,4-c]pyridin-3-one

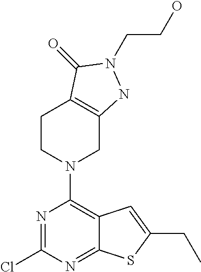

38 mg (67%) of the title compound was obtained by the same method of Preparation Example 6-41-4 except that 40 mg (0.18 mmol) of the compound obtained from Preparation Example 6-43-2 was used instead of the compound obtained from Preparation Example 6-41-3.

¹H NMR (400 MHz, CDCl₃); δ7.00 (1H, s), 4.82 (2H, s), 4.09 (2H, t), 4.00 (2H, t), 3.93 (2H, t), 2.88 (2H, q), 2.69 (2H, t), 1.33 (3H, t)

### Example 6-43

### 6-[6-Ethyl-2-(3-oxo-piperazin-1-yl)-thieno[2,3-d]pyrimidin-4-yl]-2-(2-hydroxy-ethyl)-1,2,4,5,6,7-hexahydro-pyrazolo[3,4-c]pyridin-3-one

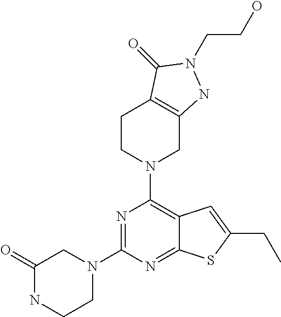

23 mg (52%) of the title compound was obtained by the same method of Example 6-1 except that 38 mg (0.1 mmol) of the compound obtained from Preparation Example 3-43-3 was used instead of the compound obtained from Preparation Example 6-1-2.

¹H NMR (400 MHz, CDCl₃+CD₃OD); δ 6.86 (1H, s), 4.68 (2H, s), 4.36 (2H, s), 4.04˜3.92 (6H, m), 3.85 (2H, t), 3.44 (2H, t), 2.84 (2H, q), 2.59 (2H, t), 1.32 (3H, t)

### Preparation Example 6-44-1

### 3-Oxo-2-phenyl-1,2,3,4,5,7-hexahydro-pyrazolo[3,4-c]pyridine-6-carboxylic acid tert-butyl ester

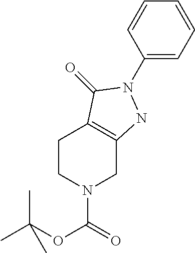

190 mg (43%) of the title compound was obtained by the same method of Preparation Example 6-41-2 except that 227 mg (2.1 mmol) of phenyl-hydrazine was used instead of methyl-hydrazine.

¹H NMR (500 MHz, CDCl₃); δ 7.86 (1H, d), 7.66 (1H, d), 7.40 (2H, t), 7.20 (1H, t), 4.42 (2H, s), 3.62 (2H, br s), 2.43 (2H, br s), 1.47 (9H, s)

### Preparation Example 6-44-2

### 2-Phenyl-1,2,4,5,6,7-hexahydro-pyrazolo[3,4-c]pyridin-3-one; hydrochloride

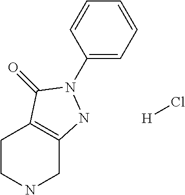

150 mg (99%) of the title compound was obtained by the same method of Preparation Example 6-41-3 except that 190 mg (0.6 mmol) of the compound obtained from Preparation Example 6-44-1 was used instead of the compound obtained from Preparation Example 6-41-2.

¹H NMR (400 MHz, CD₃OD); δ 7.63 (2H, d), 7.47 (2H, t), 7.36 (1H, t), 4.30 (2H, s), 3.50 (2H, t), 2.82 (2H, t)

### Preparation Example 6-44-3

### 6-(2-Chloro-6-ethyl-thieno[2,3-d]pyrimidin-4-yl)-2-phenyl-1,2,4,5,6,7-hexahydro-pyrazolo[3,4-c]pyridin-3-one

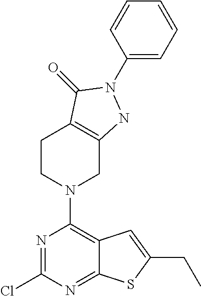

22 mg (35%) of the title compound was obtained by the same method of Preparation Example 6-41-4 except that 45 mg (0.18 mmol) of the compound obtained from Preparation Example 6-44-2 was used instead of the compound obtained from Preparation Example 6-41-3.

¹H NMR (500 MHz, CDCl₃); δ 7.70 (2H, d), 7.43 (2H, t), 7.26 (1H, t), 7.10 (1H, s), 4.91 (2H, s), 4.10 (2H, t), 2.93 (2H, q), 2.74 (2H, t), 1.37 (3H, t)

### Example 6-44

### 6-[6-Ethyl-2-(3-oxo-piperazin-1-yl)-thieno[2,3-d]pyrimidin-4-yl]-2-phenyl-1,2,4,5,6,7-hexahydro-pyrazolo[3,4-c]pyridin-3-one

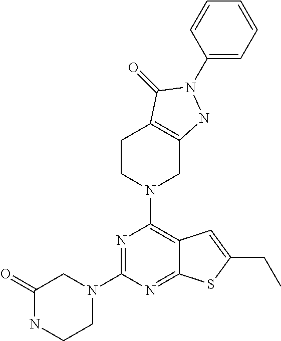

9 mg (36%) of the title compound was obtained by the same method of Example 6-1 except that 22 mg (0.053 mmol) of the compound obtained from Preparation Example 6-43-3 was used instead of the compound obtained from Preparation Example 6-1-2.

¹H NMR (500 MHz, CDCl₃); δ 7.79 (2H, d), 7.39 (2H, t), 7.18 (1H, t), 6.81 (1H, s), 6.72 (1H, br s), 4.69 (2H, br s), 4.30 (2H, s), 3.94 (2H, br s), 3.87 (2H, t), 3.39 (2H, br s), 2.82 (2H, q), 2.60 (2H, t), 1.30 (3H, t)

### Example 7-1

### Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide

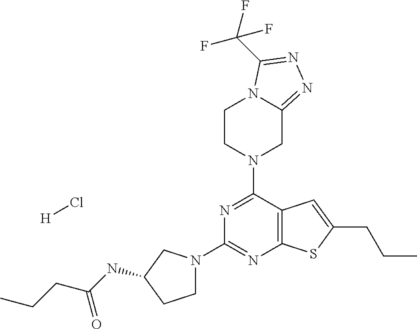

According to the same method to that described in Example 1-105, the reaction using the compound (100 mg, 0.190 mmol) obtained from Example 1-14 and butyric anhydride (0.062 ml, 0.381 mmol) was carried out. Then, the resulting mixture was treated by the same method to that described in Example 1-4 to give the title compound (71 mg, 67%).

¹H NMR (400 MHz, DMSO); δ 8.14 (1H, d), 7.36 (1H, s), 5.26 (2H, s), 4.37 (3H, br), 4.33 (3H, br), 3.67˜3.78 (3H, br), 3.44 (1H, br), 2.79 (2H, t), 2.16 (1H, m), 2.04 (2H, t), 1.90 (1H, m), 1.65 (2H, m), 1.47 (2H, m), 0.93 (3H, t), 0.83 (3H, t)

### Example 7-2

### Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide

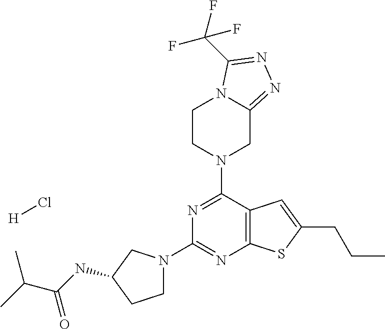

According to the same method to that described in Example 1-86, the reaction using the compound (100 mg, 0.190 mmol) obtained from Example 1-14 and isobutyric acid (0.021 ml, 0.228 mmol) was carried out. Then, the resulting mixture was treated by the same method to that described in Example 1-4 to give the title compound (71 mg, 67%).

¹H NMR (400 MHz, DMSO); δ 8.06 (1H, d), 7.34 (1H, s), 5.24 (2H, s), 4.36 (3H, br), 4.31 (3H, br), 3.64˜3.78 (3H, br), 3.39 (1H, br), 2.79 (2H, t), 2.33 (1H, m), 2.16 (1H, m), 1.88 (1H, m), 1.63 (2H, m), 0.98 (6H, d), 0.93 (3H, t)

### Example 7-3

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

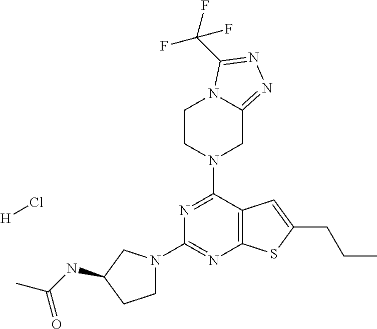

According to the same method to that described in Example 1-105, the reaction using the compound (100 mg, 0.190 mmol) obtained from Example 1-13 and acetic anhydride (0.036 ml, 0.380 mmol) was carried out. Then, the resulting mixture was treated by the same method to that described in Example 1-4 to give the title compound (75 mg, 74%).

¹H NMR (400 MHz, DMSO); δ 8.20 (1H, d), 7.36 (1H, s), 5.26 (2H, s), 4.37 (3H, br), 4.33 (3H, br), 3.65˜3.77 (3H, br), 3.39 (1H, br), 2.79 (2H, t), 2.16 (1H, m), 1.90 (1H, m), 1.82 (3H, s), 1.63 (2H, m), 0.93 (3H, t)

### Example 7-4

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide

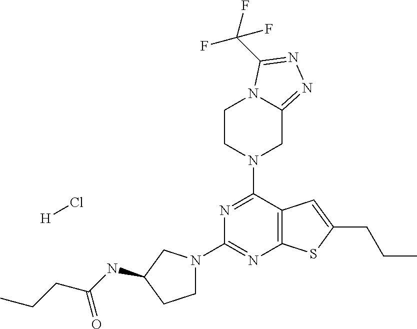

According to the same method to that described in Example 1-105, the reaction using the compound (100 mg, 0.190 mmol) obtained from Example 1-13 and butyric anhydride (0.062 ml, 0.381 mmol) was carried out. Then, the resulting mixture was treated by the same method to that described in Example 1-4 to give the title compound (69 mg, 65%).

¹H NMR (400 MHz, DMSO); δ 8.14 (1H, d), 7.36 (1H, s), 5.26 (2H, s), 4.37 (3H, br), 4.33 (3H, br), 3.67˜3.78 (3H, br), 3.44 (1H, br), 2.79 (2H, t), 2.16 (1H, m), 2.04 (2H, t), 1.90 (1H, m), 1.65 (2H, m), 1.47 (2H, m), 0.93 (3H, t), 0.83 (3H, t)

### Example 7-5

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide

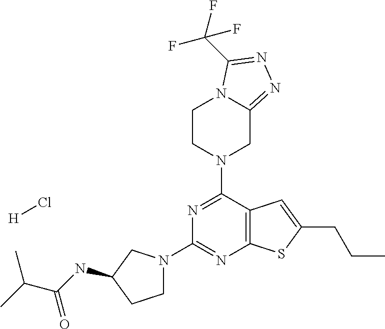

According to the same method to that described in Example 1-86, the reaction using the compound (100 mg, 0.190 mmol) obtained from Example 1-13 and isobutyric acid (0.021 ml, 0.228 mmol) was carried out. Then, the resulting mixture was treated by the same method to that described in Example 1-4 to give the title compound (71 mg, 67%).

¹H NMR (400 MHz, DMSO); δ 8.06 (1H, d), 7.34 (1H, s), 5.24 (2H, s), 4.36 (3H, br), 4.31 (3H, br), 3.64˜3.78 (3H, br), 3.39 (1H, br), 2.79 (2H, t), 2.33 (1H, m), 2.16 (1H, m), 1.88 (1H, m), 1.63 (2H, m), 0.98 (6H, d), 0.93 (3H, t)

### Example 7-6

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-acetamide

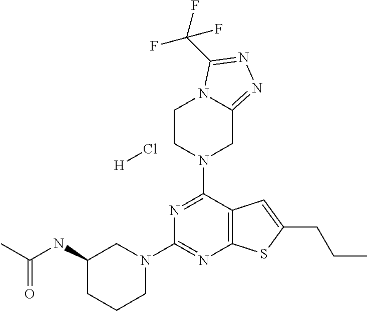

According to the same method to that described in Example 1-105, the reaction using the compound (102 mg, 0.190 mmol) obtained from Example 1-129 and acetic anhydride (0.036 ml, 0.380 mmol) was carried out. Then, the resulting mixture was treated by the same method to that described in Example 1-4 to give the title compound (75 mg, 72%).

¹H NMR (400 MHz, DMSO); δ 7.85 (1H, d), 7.26 (1H, s), 5.16 (2H, s), 4.35 (3H, br), 4.21 (3H, br), 3.66 (1H, br), 3.20 (1H, br), 3.04 (1H, br), 2.77 (2H, t), 1.76˜1.85 (5H, br), 1.62 (2H, m), 1.45 (2H, m), 0.93 (3H, t)

### Example 7-7

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-butyramide

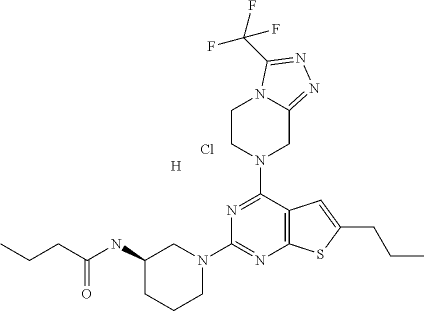

According to the same method to that described in Example 1-105, the reaction using the compound (102 mg, 0.190 mmol) obtained from Example 1-129 and butyric anhydride (0.062 ml, 0.381 mmol) was carried out. Then, the resulting mixture was treated by the same method to that described in Example 1-4 to give the title compound (70 mg, 64%).

¹H NMR (400 MHz, DMSO); δ 7.76 (1H, d), 7.25 (1H, s), 5.16 (2H, s), 4.36 (3H, br), 4.23 (3H, br), 3.68 (1H, br), 3.19 (1H, br), 3.05 (1H, br), 2.77 (2H, t), 2.02 (2H, t), 1.84 (1H, br), 1.77 (1H, br), 1.64 (2H, m), 1.47 (4H, m), 0.93 (3H, t), 0.82 (3H, t)

### Example 7-8

### Hydrochloric acid salt of N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-isobutyramide

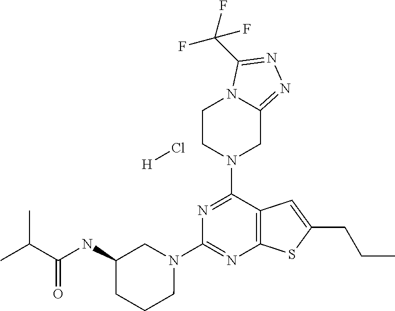

According to the same method to that described in Example 1-86, the reaction using the compound (102 mg, 0.190 mmol) obtained from Example 1-129 and isobutyric acid (0.021 ml, 0.228 mmol) was carried out. Then, the resulting mixture was treated by the same method to that described in Example 1-4 to give the title compound (20 mg, 18%).

¹H NMR (400 MHz, DMSO); δ 7.69 (1H, d), 7.26 (1H, s), 5.11 (2H, s), 4.37 (2H, br), 4.24 (4H, br), 3.67 (1H, br), 3.26 (1H, br), 3.14 (1H, br), 2.77 (2H, t), 2.34 (1H, m), 1.79 (2H, br), 1.64 (2H, m), 1.48 (2H, br), 0.93 (9H, m)

### Example 7-9

### Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-acetamide

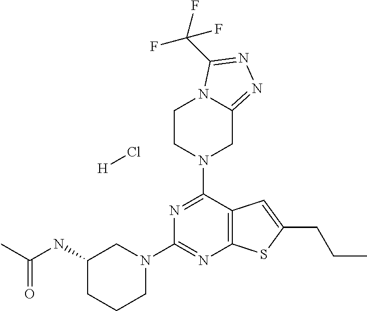

According to the same method to that described in Example 1-105, the reaction using the compound (102 mg, 0.190 mmol) obtained from Example 1-125 and acetic anhydride (0.036 ml, 0.380 mmol) was carried out. Then, the resulting mixture was treated by the same method to that described in Example 1-4 to give the title compound (73 mg, 71%).

¹H NMR (400 MHz, DMSO); δ 7.85 (1H, d), 7.26 (1H, s), 5.16 (2H, s), 4.35 (3H, br), 4.21 (3H, br), 3.66 (1H, br), 3.20 (1H, br), 3.04 (1H, br), 2.77 (2H, t), 1.76˜1.85 (5H, br), 1.62 (2H, m), 1.45 (2H, m), 0.93 (3H, t)

### Example 7-10

### Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-butyramide

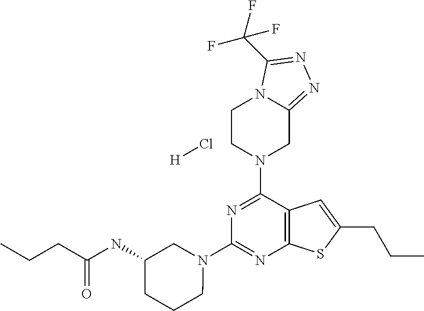

According to the same method to that described in Example 1-105, the title compound (68 mg, 62%) was obtained by using the compound (102 mg, 0.190 mmol) obtained from Example 1-125 and butyric anhydride (0.062 ml, 0.381 mmol).

¹H NMR (400 MHz, DMSO); δ 7.76 (1H, d), 7.25 (1H, s), 5.16 (2H, s), 4.36 (3H, br), 4.23 (3H, br), 3.68 (1H, br), 3.19 (1H, br), 3.05 (1H, br), 2.77 (2H, t), 2.02 (2H, t), 1.84 (1H, br), 1.77 (1H, br), 1.64 (2H, m), 1.47 (4H, m), 0.93 (3H, t), 0.82 (3H, t)

### Example 7-11

### Hydrochloric acid salt of N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-3-yl}-isobutyramide

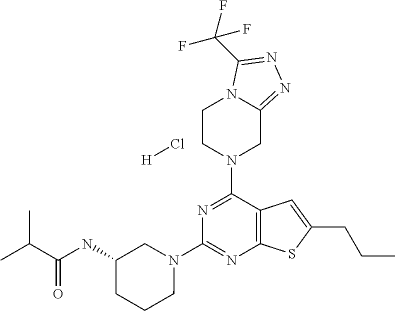

According to the same method to that described in Example 1-86, the title compound (27 mg, 25%) was obtained by using the compound (102 mg, 0.190 mmol) obtained from Example 1-125 and isobutyric acid (0.021 ml, 0.228 mmol).

¹H NMR (400 MHz, DMSO); δ 7.69 (1H, d), 7.26 (1H, s), 5.11 (2H, s), 4.37 (2H, br), 4.24 (4H, br), 3.67 (1H, br), 3.26 (1H, br), 3.14 (1H, br), 2.77 (2H, t), 2.34 (1H, m), 1.79 (2H, br), 1.64 (2H, m), 1.48 (2H, br), 0.93 (9H, m)

### Example 7-12

### Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-acetamide

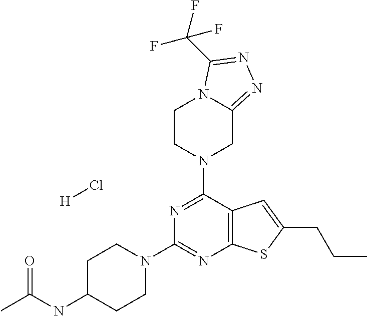

According to the same method to that described in Example 1-105, the reaction using the compound (102 mg, 0.190 mmol) obtained from Example 1-69 and acetic anhydride (0.036 ml, 0.380 mmol) was carried out. Then, the resulting mixture was treated by the same method to that described in Example 1-4 to give the title compound (85 mg, 81%).

¹H NMR (400 MHz, DMSO); δ 7.84 (1H, d), 7.29 (1H, s), 5.19 (2H, s), 4.46 (2H, d), 4.35 (2H, t), 4.27 (2H, t), 3.84 (1H, br), 3.11 (2H, t), 2.78 (2H, t), 1.80 (5H, d), 1.62 (2H, t), 1.30 (2H, m), 0.93 (3H, t)

### Example 7-13

### Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-butyramide

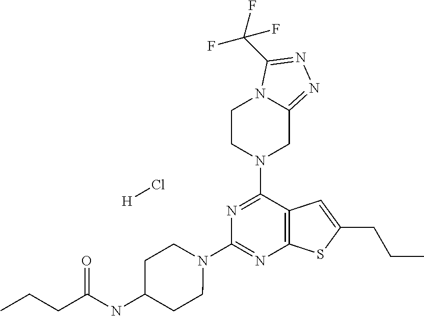

According to the same method to that described in Example 1-105, the reaction using the compound (102 mg, 0.190 mmol) obtained from Example 1-69 and butyric anhydride (0.062 ml, 0.381 mmol) was carried out. Then, the resulting mixture was treated by the same method to that described in Example 1-4 to give the title compound (66 mg, 61%).

¹H NMR (400 MHz, DMSO); δ 7.73 (1H, d), 7.27 (1H, s), 5.16 (2H, s), 4.46 (2H, d), 4.35 (2H, t), 4.24 (2H, t), 3.85 (1H, br), 3.07 (2H, t), 2.77 (2H, t), 2.01 (2H, t), 1.79 (2H, br), 1.62 (2H, m), 1.46 (2H, m), 1.29 (2H, br), 0.93 (3H, t), 0.83 (3H, t)

### Example 7-14

### Hydrochloric acid salt of N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperidin-4-yl}-isobutyramide

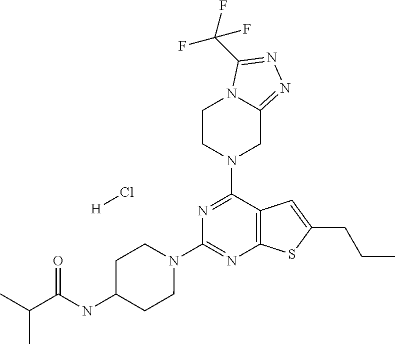

According to the same method to that described in Example 1-86, the reaction using the compound (102 mg, 0.190 mmol) obtained from Example 1-69 and isobutyric acid (0.021 ml, 0.228 mmol) was carried out. Then, the resulting mixture was treated by the same method to that described in Example 1-4 to give the title compound (26 mg, 24%).

¹H NMR (400 MHz, DMSO); δ 7.61 (1H, d), 7.23 (1H, s), 5.13 (2H, s), 4.46 (2H, d), 4.36 (2H, t), 4.21 (2H, t), 3.80 (1H, br), 3.02 (2H, t), 2.76 (2H, t), 2.28 (1H, m), 1.77 (2H, br), 1.63 (2H, m), 1.30 (2H, br), 0.98 (6H, d), 0.93 (3H, t)

### Example 7-15

### Hydrochloric acid salt of 1-{4-[6-Propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-butan-1-one

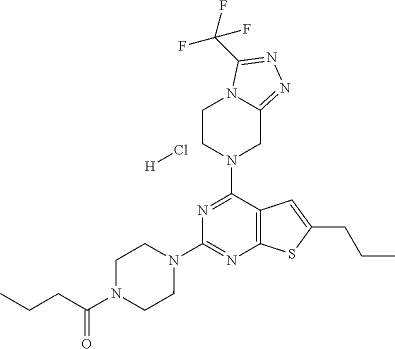

According to the same method to that described in Example 1-105, the reaction using the compound (100 mg, 0.190 mmol) obtained from Example 1-4 and butyric anhydride (0.036 ml, 0.380 mmol) was carried out. Then, the resulting mixture was treated by the same method to that described in Example 1-4 to give the title compound (30 mg, 28%).

¹H NMR (400 MHz, DMSO); δ 7.27 (1H, s), 5.17 (2H, s), 4.35 (2H, t), 4.25 (2H, t), 3.77 (2H, br), 3.72 (2H, br), 3.54 (4H, br), 2.77 (2H, t), 2.32 (2H, t), 1.64 (2H, m), 1.51 (2H, m), 0.89 (6H, m)

### Example 7-16

### Hydrochloric acid salt of 2-Methyl-1-{4-[6-propyl-4-(3-trifluoromethyl-5,6-dihydro-8H-[1,2,4]triazolo[4,3-a]pyrazin-7-yl)-thieno[2,3-d]pyrimidin-2-yl]-piperazin-1-yl}-propan-1-one;

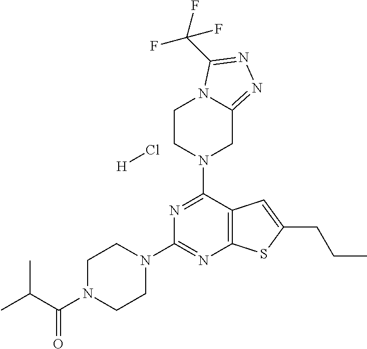

According to the same method to that described in Example 1-86, the reaction using the compound (100 mg, 0.190 mmol) obtained from Example 1-4 and isobutyric acid (0.021 ml, 0.228 mmol) was carried out. Then, the resulting mixture was treated by the same method to that described in Example 1-4 to give the title compound (29 mg, 27%).

¹H NMR (400 MHz, DMSO); δ 7.29 (1H, s), 5.19 (2H, s), 4.37 (2H, t), 4.26 (2H, t), 3.79 (2H, br), 3.74 (2H, br), 3.56 (4H, br), 2.90 (1H, m), 2.78 (2H, t), 1.64 (2H, m), 1.03 (6H, d), 0.93 (3H, t)

### Example 7-17

### N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

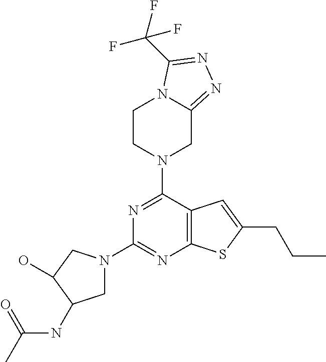

According to the same method to that described in Example 1-105, the title compound (30 mg, 16%) was obtained by using the compound (200 mg, 0.369 mmol) obtained from Example 1-114 and acetic anhydride (0.038 ml, 0.369 mmol).

¹H NMR (400 MHz, MeOD); δ 6.95 (1H, s), 5.08 (2H, s), 4.30 (2H, t), 4.18 (4H, br), 3.83 (1H, m), 3.72 (1H, m), 3.48 (2H, br), 2.74 (2H, t), 1.85 (3H, s), 1.66 (2H, m), 0.93 (3H, t)

### Example 7-18

### Acetic acid 4-acetylamino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl ester

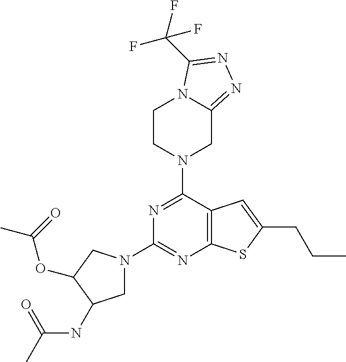

According to the same method to that described in Example 1-105, the title compound (35 mg, 17%) was obtained by using the compound (200 mg, 0.369 mmol) obtained from Example 1-114 and acetic anhydride (0.038 ml, 0.369 mmol).

¹H NMR (400 MHz, CDCl₃); δ 6.76 (1H, s), 6.23 (1H, br), 5.10 (2H, s), 4.50 (1H, d), 4.25 (4H, br), 4.13 (2H, br), 3.96 (2H, m), 3.63 (2H, m), 2.79 (2H, t), 2.06 (3H, s), 2.00 (3H, s), 1.72 (2H, m), 1.00 (3H, t)

### Example 7-19

### N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-butyramide

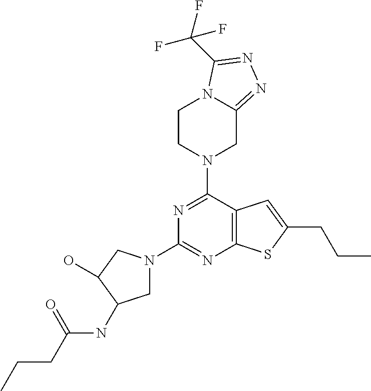

According to the same method to that described in Example 1-105, the title compound (30 mg, 15%) was obtained by using the compound (200 mg, 0.369 mmol) obtained from Example 1-114 and butyric anhydride (0.067 ml, 0.406 mmol).

¹H NMR (400 MHz, CDCl₃); δ 7.09 (1H, s), 5.21 (2H, s), 4.63 (1H, br), 4.43 (2H, t), 4.32 (4H, m), 3.96 (1H, q), 3.83 (1H, q), 3.60 (2H, br), 2.86 (2H, t), 2.21 (2H, t), 1.81 (2H, m), 1.70 (2H, m), 1.22 (3H, t), 0.97 (3H, t)

### Example 7-20

### N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-isobutyramide

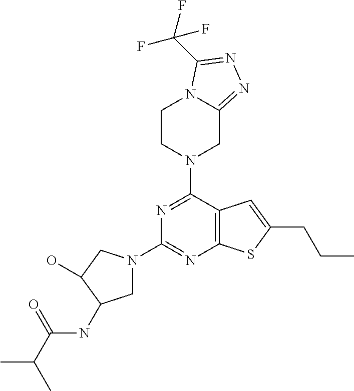

According to the same method to that described in Example 1-86, the title compound (90 mg, 45%) was obtained by using the compound (200 mg, 0.409 mmol) obtained from Example 1-114 and isobutyric acid (0.041 ml, 0.443 mmol).

¹H NMR (400 MHz, MeOD); δ 7.08 (1H, s), 5.21 (2H, s), 4.56 (1H, br), 4.43 (2H, t), 4.32 (4H, m), 3.97 (1H, q), 3.84 (1H, q), 3.62 (2H, br), 2.86 (2H, t), 2.51 (1H, m), 1.81 (2H, m), 1.20 (6H, m), 1.05 (3H, t)

### Example 7-21

### N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2,2-dimethyl-propionamide

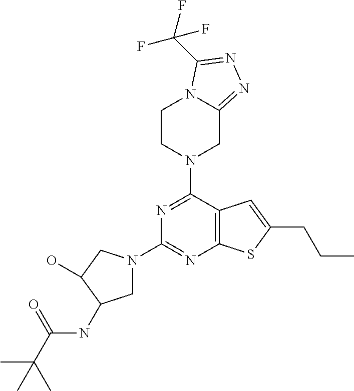

According to the same method to that described in Example 1-86, the title compound (30 mg, 15%) was obtained by using the compound (200 mg, 0.370 mmol) obtained from Example 1-114 and t-butyric acid (0.045 g, 0.443 mmol).

¹H NMR (400 MHz, CDCl₃); δ 6.78 (1H, s), 6.27 (1H, br), 5.17 (2H, s), 4.91 (1H, br), 4.34 (2H, br), 4.28 (2H, m), 4.16 (2H, br), 4.09 (1H, q), 3.95 (1H, q), 3.64 (1H, br), 3.54 (1H, br), 2.82 (2H, t), 1.79 (2H, m), 1.23 (9H, s), 1.07 (3H, t)

### Example 7-22

### 2-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

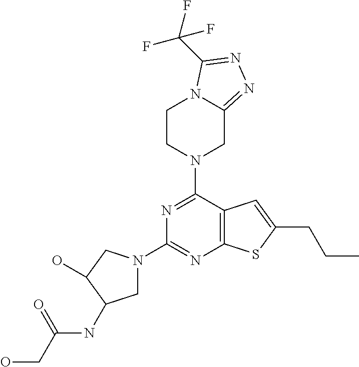

According to the same method to that described in Example 1-86, the title compound (25 mg, 13%) was obtained by using the compound (200 mg, 0.370 mmol) obtained from Example 1-114 and glycolic acid (0.034 g, 0.443 mmol).

¹H NMR (400 MHz, CDCl₃); δ 7.19 (1H, br), 6.74 (1H, s), 5.12 (2H, s), 4.60 (1H, br), 4.38 (4H, m), 4.17 (4H, br), 4.06 (1H, q), 3.95 (1H, q), 3.53 (3H, br), 2.76 (2H, t), 1.72 (2H, m), 1.00 (3H, t)

### Example 7-23

### 2-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-methyl-propionamide

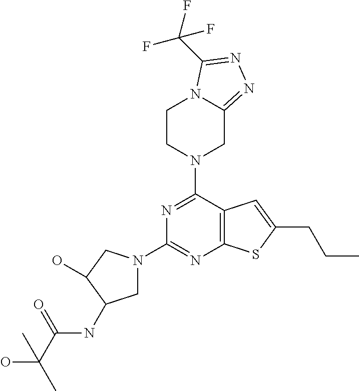

According to the same method to that described in Example 1-86, the title compound (42 mg, 20%) was obtained by using the compound (200 mg, 0.370 mmol) obtained from Example 1-114 and 2-hydroxyisobutyric acid (0.046 g, 0.443 mmol).

¹H NMR (400 MHz, CDCl₃); δ 7.30 (1H, d), 6.74 (1H, s), 5.11 (2H, s), 4.84 (1H, br), 4.35 (4H, m), 4.16 (2H, t), 4.06 (1H, q), 3.92 (1H, q), 3.84 (1H, br), 3.56 (1H, m), 2.76 (2H, t), 1.74 (2H, m), 1.44 (6H, s), 1.00 (3H, t)

### Example 7-24

### 3-Hydroxy-2-hydroxymethyl-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2-methyl-propionamide

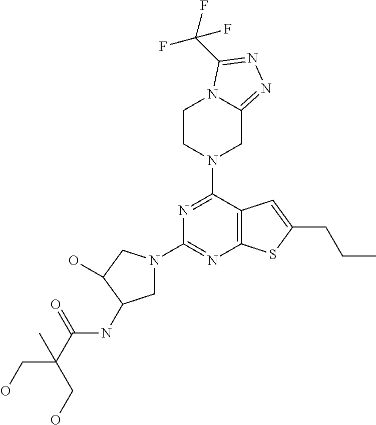

According to the same method to that described in Example 1-86, the title compound (37 mg, 17%) was obtained by using the compound (200 mg, 0.370 mmol) obtained from Example 1-114 and 2,2-bis-hydroxymethylpropionic acid (0.06 g, 0.443 mmol).

¹H NMR (400 MHz, CDCl₃); δ 7.80 (1H, br), 6.72 (1H, s), 5.19 (1H, br), 5.08 (2H, s), 4.56 (2H, br), 4.35 (2H, br), 4.26 (2H, br), 4.15 (2H, br), 4.01 (1H, br), 3.89 (1H, br), 3.77 (4H, br), 3.56 (2H, br), 2.77 (2H, t), 1.76 (2H, m), 1.11 (3H, t), 1.03 (3H, t)

### Example 7-25

### 3-Hydroxy-N-{4-hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-2,2-dimethyl-propionamide

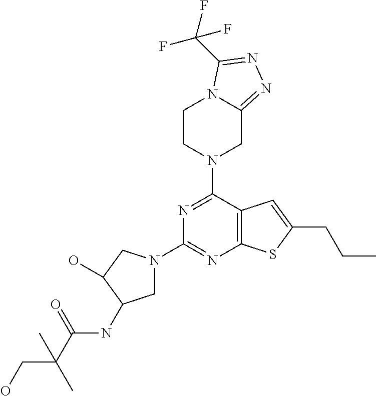

According to the same method to that described in Example 1-86, the title compound (45 mg, 21%) was obtained by using the compound (200 mg, 0.370 mmol) obtained from Example 1-114 and 2,2-dimethyl-3-hydroxypropionic acid (0.052 g, 0.443 mmol).

¹H NMR (400 MHz, CDCl₃); δ 7.11 (1H, d), 6.73 (1H, s), 5.09 (2H, s), 4.32 (4H, m), 4.11 (2H, br), 4.05 (1H, q), 3.92 (1H, q), 3.44˜3.59 (4H, m), 2.77 (2H, t), 1.75 (2H, m), 1.17 (6H, s), 1.01 (3H, t)

### Preparation Example 7-26-1

### Hydrochloric acid salt of Hexahydro-pyrrolo[3,4-d]oxazol-2-one

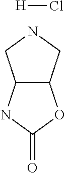

The compound (0.12 g, 0.593 mmol) obtained from Preparation Example 1-114-3 was dissolved in dichloroethane 30 mL. Diisopropylethylamine (0.31 mL, 1.78 mmol) and carbonyl diimidazole (0.192 g, 1.19 mmol) were added thereto and stirred for 16 hours. The reaction mixture was distilled under reduced pressure, diluted with ethyl acetate and washed with water and brine. The organic layer was dried with anhydrous magnesium sulfate to give the title compound (60 mg, 61.2%).

### Example 7-26

### (S)-5-[6-Propyl-4-(3-trifluoromethyl-5,6-dihydro-8H-[1,2,4]triazolo[4,3-a]pyrazin-7-yl)-thieno[2,3-d]pyrimidin-2-yl]-hexahydro-pyrrolo[3,4-d]oxazol-2-one

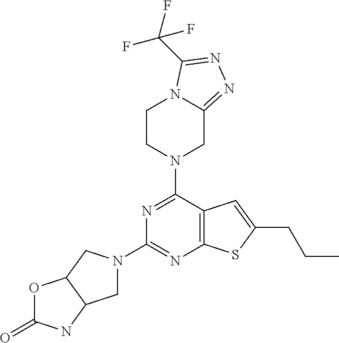

According to the same method to that described in Example 1-1, the title compound (9 mg, 5%) was obtained by using the compound (60 mg, 0.365 mmol) obtained from Preparation Example 7-26-1 and the compound (0.14 g, 0.365 mmol) obtained from Preparation Example 1-1-3.

¹H NMR (400 MHz, CDCl₃); δ 6.79 (1H, s), 6.08 (1H, br), 5.23 (1H, br), 5.11 (2H, d), 4.48 (1H, t), 4.31 (3H, m), 4.19 (2H, t), 4.10 (2H, d), 3.52 (1H, m), 3.39 (1H, m), 2.77 (2H, t), 1.68 (2H, m), 0.91 (3H, t)

### Preparation Example 7-27-1

### 3,4-Diazido-pyrrolidine-1-carboxylic acid tert-butyl ester

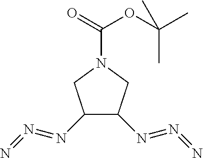

2,5-dihydro-1H-pyrrole (1.3 g, 18.70 mmol) and di-t-butyl dicarbonate (6.12 g, 28.05 mmol) were diluted in dichloromethane 30 mL and stirred for 2 hours at room temperature. The reaction mixture was distilled under reduced pressure and purified by column-chromatography using 5:1 mixture of hexane and ethyl acetate. The purified compound (3.0 g, 17.73 mmol) was dissolved in 3:1 mixture of tetrahydrofuran and water (60 ml). Osmium tetraoxide (3.61 ml, 2 mol %) and N-methylmorpholinoxide (2.91 g, 24.82 mmol) were added thereto and stirred for 16 hours at room temperature. The reaction solution was filtered through Celite and then, the filtrate was distilled under reduced pressure. The remaining residue was dissolved in dichloromethane 80 mL and cooled to 0° C. Diisopropylethylamine (9.77 ml, 56.09 mmol) and methanesulfonylchloride (3.18 ml, 41.13 mmol) was slowly added thereto. The reaction was carried out for 2 hours at room temperature, followed by washing with water and brine. After the organic layer was dried with anhydrous magnesium sulfate, distilled under reduced pressure and diluted with dimethylformamide 40 mL, sodium azide (7.29 g, 112.18 mmol) was added to the reaction solution and then, the reaction was carried out for 16 hours at 80° C. The reaction mixture was distilled under reduced pressure, diluted with ethyl acetate and washed with water and brine. After the organic layer was dried with anhydrous magnesium sulfate and distilled under reduced pressure, the title compound (2.6 g, step 4: 54%) was obtained by column-chromatography using 5:1 mixture of hexane and ethyl acetate.

¹H NMR (400 MHz, CDCl₃); δ 4.15 (2H, t), 3.75 (2H, t), 3.38˜3.50 (2H, m), 1.46 (9H, s)

### Preparation Example 7-27-2

### Tri-hydrochloric acid salt of 3,4-Diamino-pyrrolidine-1-carboxylic acid tert-butyl ester

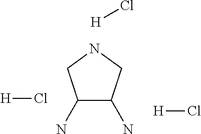

The compound (2.5 g, 9.716 mmol) obtained from Preparation Example 7-28-1 was dissolved in methanol 40 mL. Pd/C (palladium on charcoal) (0.25 g, 10 wt %) was added thereto for hydgrogenation. The reaction mixture was filtered through Celite and evaporated under reduced pressure. The remaining residue was diluted with dichloromethane 30 mL. 4.0 M hydrochloric acid dioxane solution 10 mL was added thereto and stirred for 1 hour. After the solvent was removed by distillation under reduced pressure and solidified, the title compound (1.5 g, 73%) was obtained by cleanse the solid with diethyl ether.

MS (M+1): 102.1

### Example 7-27

### Di-hydrochloric acid salt of

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3,4-diamine

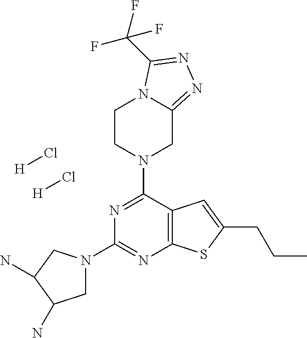

According to the same method to that described in Example 1-1, the reaction was carried out by using the compound (560 mg, 1.393 mmol) obtained from Preparation Example 1-1-3, the compound (440 mg, 2.09 mmol) obtained from Preparation Example 7-28-2 and diisobutylamine (1.21 ml, 6.965 mmol). After the reaction mixture was distilled under reduced pressure to removed the solvent, di-t-butyl dicarbonate (0.912 g, 4.179 mmol) was added thereto, diluted with dichlorometane 30 mL and stirred for 2 hours at room temperature. After the reaction solution was distilled under reduced pressure, the purification was carried out by column-chromatography using 4:1 mixture of dichloromethane and ethyl acetate. The remaing residue was diluted with dichloromethane 30 mL. 4.0 M hydrochloric acid dioxane solution 10 mL was added thereto and stirred for 1 hour. After the solvent was removed by distillation under reduced pressure and solidified, the title compound (230 mg, 31%) was obtained by cleanse the solid with diethyl ether.

¹H NMR (400 MHz, DMSO); δ 6.82 (1H, s), 5.22 (2H, s), 4.80 (2H, br), 4.38 (1H, br), 4.37 (2H, t), 4.25 (2H, t), 3.95 (2H, br), 3.50 (2H, br), 2.81 (2H, t), 1.76 (2H, m), 1.04 (3H, t)

### Example 7-28

### N-{4-Acetylamino-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

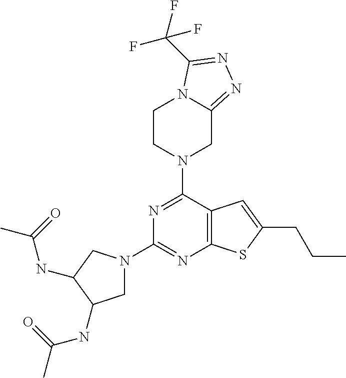

According to the same method to that described in Example 1-105, the title compound (50 mg, 33%) was obtained by using the compound (150 mg, 0.278 mmol) obtained from Example 7-28 and acetic anhydride (0.053 g, 0.556 mmol).

¹H NMR (400 MHz, CDCl₃); δ 7.46 (2H, br), 6.62 (1H, s), 4.78 (2H, br), 4.50 (2H, br), 3.90 (6H, br), 3.48 (2H, br), 2.82 (2H, t), 2.08 (6H, s), 1.78 (2H, m), 1.10 (3H, t)

### Example 7-29

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

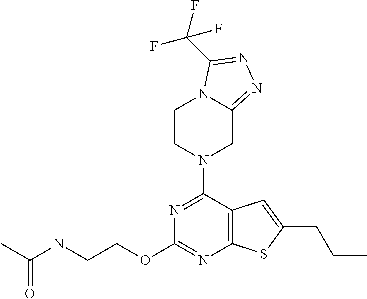

According to the same method to that described in Example 1-105, the title compound (51 mg, 57%) was obtained by using the compound (88 mg, 0.190 mmol) obtained from Example 3-58 and acetic anhydride (0.036 g, 0.380 mmol).

¹H NMR (400 MHz, CDCl₃); δ 6.95 (1H, s), 6.07 (1H, br), 5.31 (2H, s), 4.44 (2H, t), 4.37 (2H, t), 4.33 (2H, t), 3.66 (2H, m), 2.82 (2H, t), 1.99 (3H, s), 1.71 (2H, m), 1.00 (3H, t)

### Example 7-30

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-butyramide

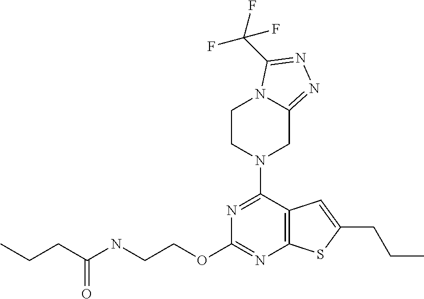

According to the same method to that described in Example 1-105, the title compound (61 mg, 65%) was obtained by using the compound (88 mg, 0.190 mmol) obtained from Example 3-58 and butyric anhydride (0.062 g, 0.380 mmol).

¹H NMR (400 MHz, CDCl₃); δ 6.95 (1H, s), 6.06 (1H, br), 5.31 (2H, s), 4.44 (2H, t), 4.37 (2H, t), 4.33 (2H, t), 3.68 (2H, m), 2.82 (2H, t), 2.14 (2H, t), 1.73 (2H, m), 1.61 (2H, m), 1.00 (3H, t), 0.91 (3H, t)

### Example 7-31

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-isobutyramide

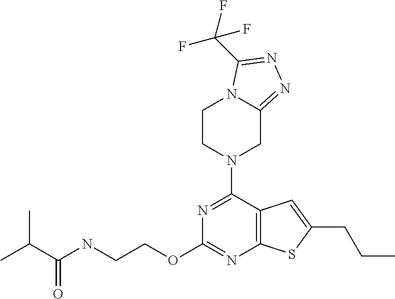

According to the same method to that described in Example 1-86, the title compound (10 mg, 11%) was obtained by using the compound (100 mg, 0.190 mmol) obtained from Example 3-58 and isobutyric acid (0.021 mL, 0.228 mmol).

¹H NMR (400 MHz, CDCl₃); δ 6.95 (1H, s), 6.09 (1H, br), 5.32 (2H, s), 4.45 (2H, t), 4.37 (2H, t), 4.33 (2H, t), 3.68 (2H, m), 2.82 (2H, t), 2.32 (1H, m), 1.73 (2H, m), 1.14 (6H, d), 0.87 (3H, t)

### Example 7-32

### 2-Hydroxy-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

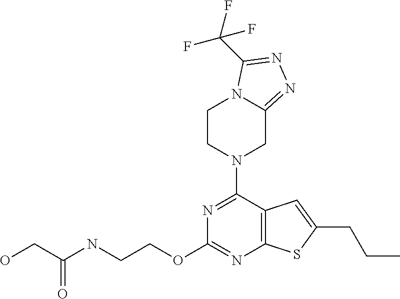

According to the same method to that described in Example 1-86, the title compound (40 mg, 40%) was obtained by using the compound (100 mg, 0.206 mmol) obtained from Example 3-58 and glycolic acid (0.019 g, 0.247 mmol).

¹H NMR (400 MHz, CDCl₃); δ 7.36 (1H, t), 6.92 (1H, s), 5.27 (2H, s), 4.48 (2H, t), 4.38 (5H, d), 4.12 (2H, br), 3.74 (2H, m), 2.83 (2H, t), 1.78 (2H, m), 1.02 (3H, t)

### Example 7-33

### 2-Hydroxy-2-methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide

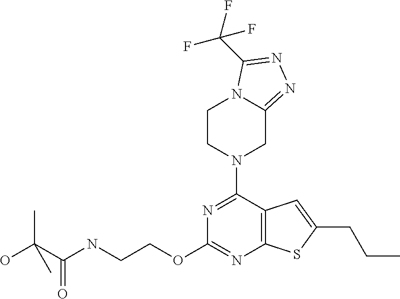

According to the same method to that described in Example 1-86, the title compound (65 mg, 61%) was obtained by using the compound (100 mg, 0.206 mmol) obtained from Example 3-58 and 2-hydroxyisobutyric acid (0.026 g, 0.247 mmol).

¹H NMR (400 MHz, CDCl₃); δ 7.41 (1H, br), 6.72 (1H, s), 5.29 (2H, s), 4.48 (2H, t), 4.38 (4H, m), 3.71 (2H, m), 3.14 (1H, br), 2.84 (2H, t), 1.79 (2H, m), 1.44 (6H, s), 0.85 (3H, t)

### Example 7-34

### 3-Hydroxy-2-hydroxymethyl-2-methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide

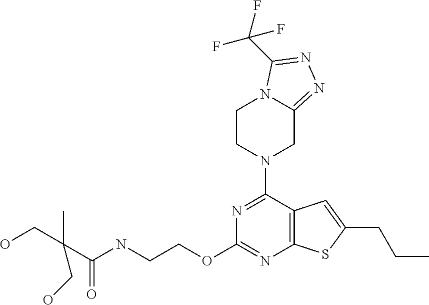

According to the same method to that described in Example 1-86, the title compound (30 mg, 27%) was obtained by using the compound (100 mg, 0.206 mmol) obtained from Example 3-58 and 2,2-bis-hydroxymethylpropionic acid (0.033 g, 0.247 mmol).

¹H NMR (400 MHz, CDCl₃); δ 7.46 (1H, br), 6.97 (1H, s), 5.29 (2H, s), 4.49 (2H, t), 4.37 (4H, m), 3.78 (6H, m), 3.64 (2H, br), 3.02 (1H, br), 2.84 (2H, t), 1.79 (2H, m), 1.06 (3H, s), 0.90 (3H, t)

### Example 7-35

### 3-Hydroxy-2,2-dimethyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-propionamide

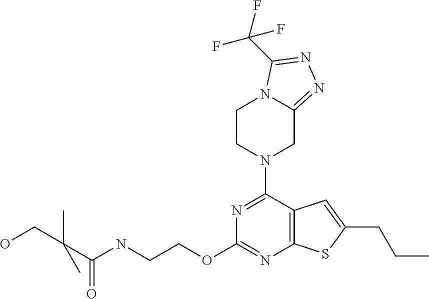

According to the same method to that described in Example 1-86, the title compound (70 mg, 64%) was obtained by using the compound (100 mg, 0.206 mmol) obtained from Example 3-58 and 2,2-dimethyl-3-hydroxypropionic acid (0.03 g, 0.247 mmol).

¹H NMR (400 MHz, CDCl₃); ¹H NMR (500 MHz, CDCl₃); δ 6.99 (1H, s), 6.68 (1H, br), 5.36 (2H, s), 4.54 (2H, t), 4.42 (4H, m), 3.75 (2H, q), 3.58 (2H, d), 3.16 (1H, t), 2.90 (2H, t), 1.82 (2H, m), 1.21 (6H, s), 0.90 (3H, t)

### Preparation Example 7-36-1

### (R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

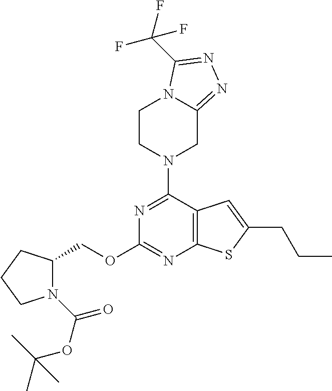

According to the same method to that described in Example 1-45, the title compound (424 mg, 14%) was obtained by using the compound (2.23 mg, 5.54 mmol) obtained from Preparation Example 1-1-3 and (R)-2-hydroxymethyl-pyrrolidine-1-carboxylic acid tert-butyl ester (2.23 g, 11.08 mmol).

¹H NMR (400 MHz, CDCl₃); δ 6.94 (1H, s), 5.32 (2H, s), 4.39 (4H, br), 4.22 (3H, br), 3.41 (2H, br), 2.81 (2H, t), 1.74˜1.96 (4H, br), 1.72 (2H, m), 1.46 (9H, s), 0.99 (3H, t)

### Example 7-37

### Hydrochloric acid salt of

### 7-[6-Propyl-2-((R)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine

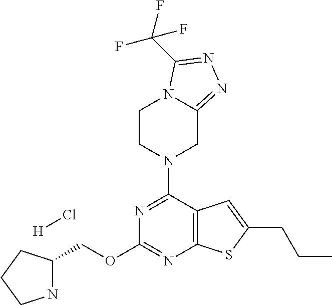

The compound (424 mg, 0.747 mmol) obtained from Preparation Example 7-36-1 was diluted in dichloromethane 30 mL. 4.0 M hydrochloric acid 4 mL was added thereto and stirred for 1 hour. After the solvent was removed by distillation under reduced pressure and solidified, the title compound (370 mg, 98%) was obtained by cleanse the solid with diethyl ether.

¹H NMR (400 MHz, DMSO); δ 9.40 (1H, br), 8.93 (1H, br), 7.42 (1H, s), 5.21 (2H, s), 4.53 (1H, m), 4.40 (1H, m), 4.33 (2H, t), 4.28 (2H, t), 3.89 (1H, br), 3.17 (2H, br), 2.80 (2H, t), 2.09 (1H, m), 1.88 (2H, m), 1.64 (3H, m), 0.91 (3H, t)

### Preparation Example 7-37-1

### (S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

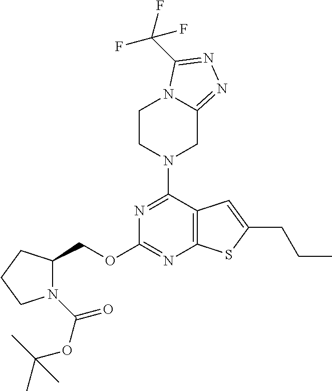

According to the same method to that described in Example 1-45, the title compound (1.2 g, 40%) was obtained by using the compound (2.13 mg, 5.29 mmol) obtained from Preparation Example 1-1-3 and (S)-2-hydroxymethyl-pyrrolidine-1-carboxylic acid tert-butyl ester (2.13 g, 10.59 mmol).

¹H NMR (400 MHz, CDCl₃); δ 6.94 (1H, s), 5.32 (2H, s), 4.39 (4H, br), 4.22 (3H, br), 3.41 (2H, br), 2.81 (2H, t), 1.74˜1.96 (4H, br), 1.72 (2H, m), 1.46 (9H, s), 0.99 (3H, t)

### Example 7-37

### Hydrochloric acid salt of

### 7-[6-Propyl-2-((S)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine

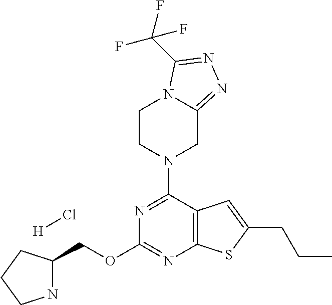

The compound (400 mg, 0.705 mmol) obtained from Preparation Example 7-37-1 was diluted in dichloromethane 30 mL. 4.0 M hydrochloric acid 4 mL was added thereto and stirred for 1 hour. After the solvent was removed by distillation under reduced pressure and solidified, the title compound (355 mg, 100%) was obtained by cleanse the solid with diethyl ether.

¹H NMR (400 MHz, DMSO); δ 9.40 (1H, br), 8.93 (1H, br), 7.42 (1H, s), 5.21 (2H, s), 4.53 (1H, m), 4.40 (1H, m), 4.33 (2H, t), 4.28 (2H, t), 3.89 (1H, br), 3.17 (2H, br), 2.80 (2H, t), 2.09 (1H, m), 1.88 (2H, m), 1.64 (3H, m), 0.91 (3H, t)

### Preparation Example 7-38-1

### {2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-carbamic acid tert-butyl ester

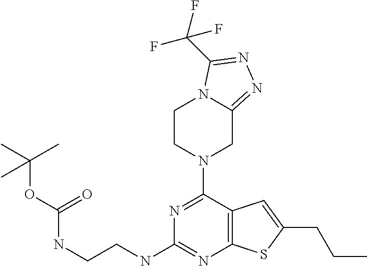

According to the same method to that described in Example 1-45, the title compound (650 mg, 16%) was obtained by using the compound (2.13 mg, 5.29 mmol) obtained from Example 1-1-3 and (2-amino-methyl)-carbamic acid tert-butyl ester (3.02 g, 7.49 mmol).

¹H NMR (400 MHz, CDCl₃); δ 6.79 (1H, s), 5.19 (2H, s), 4.33 (2H, t), 4.22 (2H, t), 3.51 (2H, t), 3.34 (2H, t), 2.76 (2H, t), 1.69 (2H, m), 1.42 (9H, s), 0.98 (3H, t)

### Example 7-38

### Hydrochloric acid salt of N-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine

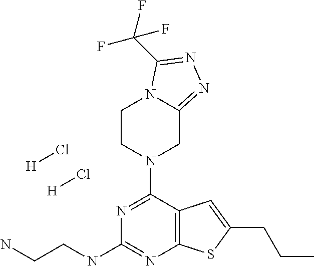

The compound (650 mg, 1.234 mmol) obtained from Preparation Example 7-38-1 was diluted in dichloromethane 30 mL. 4.0 M hydrochloric acid 4 mL was added thereto and stirred for 1 hour. After the solvent was removed by distillation under reduced pressure and solidified, the title compound (580 mg, 94%) was obtained by cleanse the solid with diethyl ether.

¹H NMR (400 MHz, DMSO); δ 8.17 (3H, br), 7.38 (1H, s), 5.22 (2H, s), 4.37 (4H, br), 3.57 (2H, br), 3.00 (2H, br), 2.80 (2H, t), 1.64 (2H, m), 0.93 (3H, t)

### Preparation Example 8-1-1

### (R)-3-Cyano-pyrrolidine-1-carboxylic acid tert-butyl ester

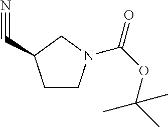

(S)-Pyrrolidin-3-ol (2 g, 22.96 mmol) and di-t-butyl dicarbonate (7.515 g, 34.44 mmol) were diluted in dichloromethane 50 mL and stirred for 2 hours at room temperature. After the reaction solution was distilled under reduced pressure, the column-chromatography using 20:1 mixture of dichloromethane and methanol was carried out for purification. The purified compound (4.3 g, 22.96 mmol) was dissolved in dichloromethane 70 mL and cooled to 0° C. Diisopropyl ethylamine (6 mL, 34 mmol) and methanesulfonylchloride (1.93 mL, 25 mmol) were slowly added thereto. The reaction was carried out for 2 hours at room temperature and the reaction mixture was washed with water and brine. After the organic layer was dried with anhydrous magnesium sulfate and distilled under reduced pressure, the remaining residue was diluted with dimethylformamide 70 mL. Sodium cyanide (3.38 g, 69 mmol) was added to the reaction solution and the reaction was carried out for 16 hours at 80° C. The resulting mixture was distilled under reduced pressure, diluted with ethyl acetate and washed with water and brine. After the organic layer was dried with anhydrous magnesium sulfate and distilled under reduced pressure, the title compound (2 g, step 3: 44%) was obtained by column-chromatography using 1:1 mixture of hexane and ethyl acetate.

¹H NMR (400 MHz, CDCl₃); δ 3.68 (1H, br), 3.58 (2H, br), 3.45 (1H, br), 3.12 (1H, m), 2.31 (1H, m), 2.22 (1H, br), 1.47 (9H, s)

### Preparation Example 8-1-2

### (R)-Pyrrolidine-1,3-dicarboxylic acid 1-tert-butyl ester 3-methyl ester

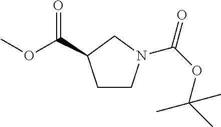

The compound (2 g, 10 mmol) obtained from Preparation Example 8-1-1 was dissolved in hydrochloric acid 10 mL and the reaction was carried out for 4 hours at 100° C. The remaining residue obtained by distillation of the reaction solution under reduced pressure was dissolved in methanol 20 mL and cooled to 0° C. Trimethylsilylchloride (5.17 mL, 40.8 mmol) was slowly added thereto. The reaction was carried out for 16 hours at room temperature and the solvent was removed by distillation under reduced pressure. The reaction mixture was diluted with dichloromethane 50 mL and diisopropylethylamine (14 mL, 80 mmol) and di-t-butyl dicarbonate (2.4 g, 11 mmol) was added thereto and stirred for 2 hours at room temperature. The reaction mixture was distilled under reduced pressure, diluted with ethyl acetate and washed with water and brine. After the organic layer was dried with anhydrous magnesium sulfate and distilled under reduced pressure, the title compound (1.5 g, step 3: 65%) was obtained by column-chromatography using 3:1 mixture of hexane and ethyl acetate.

¹H NMR (400 MHz, CDCl₃); δ 3.69 (3H, s), 3.45˜3.66 (3H, br), 3.33 (1H, br), 3.04 (1H, br), 2.11 (2H, br), 1.44 (9H, s)

### Preparation Example 8-1-3

### (R)-3-Hydroxymethyl-pyrrolidine-1-carboxylic acid tert-butyl ester

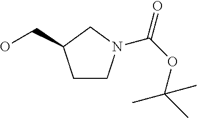

The compound (1.5 g, 6.542 mmol) obtained from Preparation Example 8-1-2 was dissolved in tetrahydrofuran 40 mL and cooled to 0° C. Litium borohydride 2.0 M tetrahydrofuran solution (6.54 mL, 13.08 mmol) was slowly added thereto and the reaction was carried out for 16 hours at room temperature. The reaction solution was neutralized with 1 N hydrochloric acid solution, distilled under reduced pressure, diluted with ethyl acetate and washed with water and brine. The organic layer was dried with anhydrous magnesium sulfate to give the title compound (1.0 g, 76%).

¹H NMR (400 MHz, CDCl₃); δ 3.65 (2H, br), 3.36˜3.53 (3H, br), 3.15 (1H, br), 2.43 (1H, br), 2.00 (2H, br), 1.65 (1H, br), 1.49 (9H, s)

### Preparation Example 8-1-4

### (S)-3-Aminomethyl-pyrrolidine-1-carboxylic acid tert-butyl ester

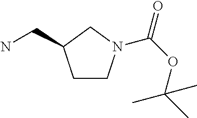

The compound (2.26 g, 11.23 mmol) obtained from Preparation Example 8-1-3 was dissolved in dichloromethane 80 mL and cooled to 0° C. Diisopropylamine (2.94 mL, 16.84 mmol) and methanesulfonylchloride (0.96 mL, 12.35 mmol) were slowly added thereto. The reaction was carried out for 2 hours at room temperature and the reaction mixture was washed with water and brine. After the organic layer was dried with anhydrous magnesium sulfate and distilled under reduced pressure, the remaining residue was diluted with dimethylformamide 50 mL. Sodium azide (2.19 g, 12.35 mmol) was added thereto and the reaction was carried out for 16 hours at 80° C. The reaction mixture was distilled under reduced pressure and diluted with ethyl acetate and washed with water and brine. After the organic layer was dried with anhydrous magnesium sulfate and distilled under reduced pressure, the column-chromatography using 5:1 mixture of hexane and ethyl acetate was carried out for purification. The purified compound (2 g, 8.84 mmol) was dissolved in methanol 20 mL and Pd/C (palladium on charcoal) (0.2 g, 10 wt %) was added thereto for hydrogenation. After the reaction mixture was filtered through Celite, the title compound (2 g, 89%) was obtained by evaporation under reduced pressure without further purification.

### Preparation Example 8-1-5

### Dihydrochloric acid salt of C—(S)-1-Pyrrolidin-3-yl-methylamine

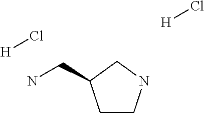

The compound 1.2 g (5.99 mmol) obtained from Preparation Example 8-1-4 was diluted with dichloromethane 30 mL. 4.0 M hydrochloric acid dioxane solution 10 mL was added thereto and stirred for 1 hour. After the solvent was removed by distillation under reduced pressure and solidified, the title compound (1.0 g, 96%) was obtained by cleanse the solid with diethyl ether.

MS (M+1): 101.2

### Preparation Example 8-1-6

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-carbamic acid tert-butyl ester

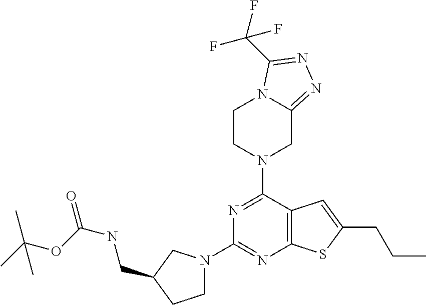

According to the same method to that described in Example 1-1, the reaction using the compound (1.3 g, 3.25 mmol) obtained from Preparation Example 1-1-3, the compound (1 g, 4.43 mmol) obtained from Preparation Example 8-1-5 and diisobutylamine (2.83 mL, 16.26 mmol) was carried out. After the reaction mixture was distilled under reduced pressure to remove the solvent, di t-butyl dicarbonate (1.42 g, 6.50 mmol) was added thereto, diluted with dichloromethane 30 mL and stirred for 2 hours. After the organic layer was dried with anhydrous magnesium sulfate and distilled under reduced pressure, the title compound (400 mg, step 2: 22%) was obtained by column-chromatography using 4:1 mixture of dichloromethane and ethyl acetate.

¹H NMR (400 MHz, DMSO); δ 6.76 (1H, s), 5.18 (2H, s), 4.68 (1H, br), 4.35 (2H, t), 4.19 (2H, t), 3.74 (2H, m), 3.55 (1H, m), 3.27 (2H, m), 3.19 (1H, m), 2.79 (2H, t), 2.50 (1H, m), 2.10 (1H, m), 1.75 (2H, m), 1.45 (9H, s), 1.01 (3H, t)

### Preparation Example 8-1-7

### Hydrochloric acid salt of C-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methylamine

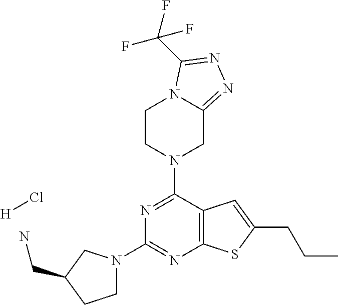

The compound (400 mg, 0.706 mmol) obtained from Preparation Example 8-1-6 was diluted with dichloromethane 30 mL. 4.0 M hydrochloric acid dioxane solution 4 mL was added thereto and stirred for 1 hour. After the solvent was removed by distillation under reduced pressure and solidified, the title compound (350 mg, 98%) was obtained by cleanse the solid with diethyl ether.

MS (M+1): 467.4

### Example 8-1

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-acetamide

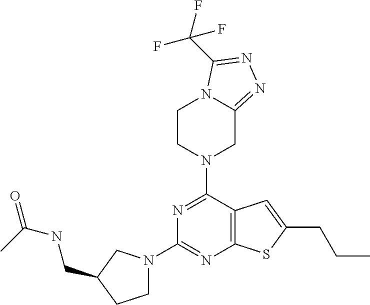

According to the same method to that described in Example 1-105, the title compound (60 mg, 57%) was obtained by using the compound (100 mg, 0.206 mmol) obtained from Preparation Example 8-1-7 and acetic anhydride (0.02 mL, 0.206 mmol).

¹H NMR (400 MHz, CDCl₃); δ 6.82 (1H, s), 6.30 (1H, br), 5.14 (2H, s), 4.35 (2H, t), 4.20 (2H, t), 3.76 (2H, m), 3.53 (1H, m), 3.43 (1H, m), 3.29 (2H, m), 2.78 (2H, t), 2.58 (1H, m), 2.18 (1H, m), 2.02 (3H, s), 1.77 (3H, m), 0.99 (3H, t)

### Example 8-2

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-butyramide

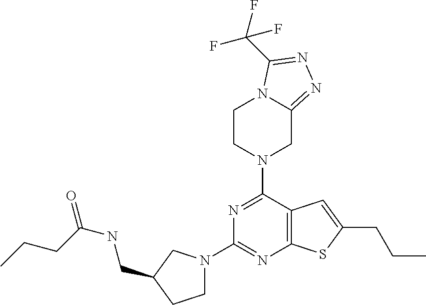

According to the same method to that described in Example 1-105, the title compound (45 mg, 41%) was obtained by using the compound (100 mg, 0.206 mmol) obtained from Preparation Example 8-1-7 and butyric anhydride (0.034 mL, 0.206 mmol).

¹H NMR (400 MHz, CDCl₃); δ 6.78 (1H, s), 5.92 (1H, br), 5.17 (2H, s), 4.36 (2H, t), 4.22 (2H, t), 3.78 (2H, m), 3.57 (2H, m), 3.31 (2H, m), 2.80 (2H, t), 2.58 (1H, m), 2.22 (2H, m), 2.14 (1H, m), 1.79 (5H, m), 1.03 (6H, m)

### Example 8-3

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-isobutyramide

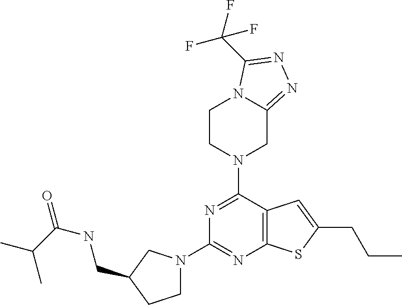

According to the same method to that described in Example 1-86, the title compound (50 mg, 45%) was obtained by using the compound (100 mg, 0.206 mmol) obtained from Preparation Example 8-1-7 and isobutyric acid (0.023 mL, 0.247 mmol).

¹H NMR (400 MHz, CDCl₃); δ 6.78 (1H, s), 5.88 (1H, br), 5.17 (2H, s), 4.36 (2H, t), 4.26 (2H, t), 3.78 (2H, m), 3.57 (2H, m), 3.31 (2H, m), 2.82 (2H, t), 2.60 (1H, m), 2.45 (1H, m), 2.16 (1H, m), 1.79 (3H, m), 1.20 (6H, d), 1.03 (3H, t)

### Preparation Example 8-4-1

### (S)-3-Cyano-pyrrolidine-1-carboxylic acid tert-butyl ester

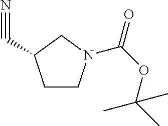

(R)-pyrrolidin-3-ol 7.7 g (62.31 mmol) and di-t-butyl dicarbonate (20.40 g, 93.46 mmol) were diluted with dichloromethane 50 mL and stirred for 2 hours at room temperature. After the reaction solution was distilled under reduced pressure, the column-chromatography using 20:1 mixture of dichloromethane and methanol was carried out for purification. The purified compound (11.4 g, 61.0 mmol) was dissolved in dichloromethane 70 mL and cooled to 0° C. Diisopropylethylamine (15.904 mL, 91.3 mmol) and methanesulfonylchloride (5.19 mL, 67 mmol) were slowly added thereto. The reaction was carried out for 2 hours at room temperature and the reaction mixture was washed with water and brine. After the organic layer was dried with anhydrous magnesium sulfate and distilled under reduced pressure, the remaining residue was diluted with dimethylformamide 100 mL. Sodium cyanide (8.97 g, 183 mmol) was added thereto and the reaction was carried out for 16 hours at 80° C. The reaction mixture was distilled under reduced pressure, diluted with ethyl acetate and washed with water and brine. After the organic layer was dried with anhydrous magnesium sulfate and distilled under reduced pressure, the title compound (3.7 g, step 3: 30.3%) was obtained by column-chromatography using 1:1 mixture of hexane and ethyl acetate.

¹H NMR (400 MHz, CDCl₃); δ 3.68 (1H, br), 3.58 (2H, br), 3.45 (1H, br), 3.12 (1H, m), 2.31 (1H, m), 2.22 (1H, br), 1.47 (9H, s)

### Preparation Example 8-4-2

### (S)-Pyrrolidine-1,3-dicarboxylic acid 1-tert-butyl ester 3-methyl ester

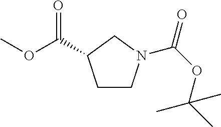

The compound (3 g, 15.29 mmol) obtained from Preparation Example 8-4-1 was dissolved in hydrochloric acid 18 mL and the reaction was carried out for 4 hours at 100° C. After the reaction solution was distilled under reduced pressure, the remaining residue was dissolved in methanol 20 mL and cooled to 0° C. Trimethylsilylchloride (7.76 mL, 61.15 mmol) was slowly added thereto. After the reaction was carried out for 16 hours at room temperature, the solvent was removed by distillation under reduced pressure. The reaction mixture was diluted with dichloromethane 50 mL. Diisopropylethylamine (21.30 mL, 122.3 mmol) and di t-butyl dicarbonate (3.67 g, 16.82 mmol) were added thereto and stirred for 2 hours at room temperature. The reaction mixture was distilled under reduced pressure, diluted with ethyl acetate and washed with water and brine. After the organic layer was dried with anhydrous magnesium sulfate and distilled under reduced pressure, the title compound (2.6 g, step 3: 74%) was obtained by column-chromatography using 3:1 mixture of hexane and ethyl acetate.

¹H NMR (400 MHz, CDCl₃); δ 3.69 (3H, s), 3.45˜3.66 (3H, br), 3.33 (1H, br), 3.04 (1H, br), 2.11 (2H, br), 1.44 (9H, s)

### Preparation Example 8-4-3

### (S)-3-Hydroxymethyl-pyrrolidine-1-carboxylic acid tert-butyl ester

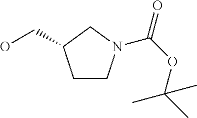

The compound (2.6 g, 11.34 mmol) obtained from Preparation Example 8-4-2 was dissolved in tetrahydrofuran 40 mL and cooled to 0° C. Lithium borohydride 2.0 M tetrahydrofuran solution (11.34 mL, 22.68 mmol) was slowly added thereto and the reaction was carried out for 16 hours at room temperature. The reaction solution was neutralized with 1N hydrochloric acid, distilled under reduced pressure, diluted with ethyl acetate and washed with water and brine. The organic layer was dried with anhydrous magnesium sulfate to give the title compound (1.1 g, 48%).

¹H NMR (400 MHz, CDCl₃); δ 3.65 (2H, br), 3.36˜3.53 (3H, br), 3.15 (1H, br), 2.43 (1H, br), 2.00 (2H, br), 1.65 (1H, br), 1.49 (9H, s)

### Preparation Example 8-4-4

### (R)-3-Aminomethyl-pyrrolidine-1-carboxylic acid tert-butyl ester

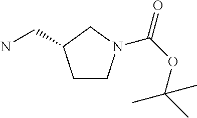

The compound (6.0 g, 29.81 mmol) obtained from Preparation Example 8-4-3 was dissolved in dichloromethane 100 mL and cooled to 0° C. Diisopropylethylamine (7.8 mL, 44.72 mmol) and methanesulfonylchloride (2.54 mL, 32.79 mmol) were slowly added thereto. The reaction was carried out for 2 hours at room temperature and then, the reaction mixture was washed with water and brine. After the organic layer was dried with anhydrous magnesium sulfate and distilled under reduced pressure, the remaining residue was diluted with dimethylformamide 100 mL. Sodium azide (5.814 g, 89.43 mmol) was added to the reaction solution and the reaction was carried out for 16 hours at 80° C. The reaction mixture was distilled under reduced pressure, diluted with ethyl acetate and washed with water and brine. After the organic layer was dried with anhydrous magnesium sulfate and distilled under reduced pressure, the column-chromatography using 5:1 mixture of hexane and ethyl acetate was carried out for purification. The purified compound (4.5 g, 19.9 mmol) was dissolved in methanol 50 mL. Pd/C (palladium on charcoal) (0.5 g, 10 wt %) was added thereto for hydrogenation. After the reaction mixture was filtered through Celite, the title compound (4 g, 67%) was obtained by evaporation under reduced pressure without additional purification.

### Preparation Example 8-4-5

### Dihydrochloric acid salt of C—(R)-1-Pyrrolidin-3-yl-methylamine

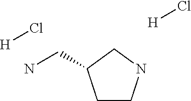

The compound (1.5 g, 7.49 mmol) obtained from Preparation Example 8-4-4 was dissolved in dichloromethane 30 mL. 4.0 M hydrochloric acid dioxane solution 10 mL was added thereto and stirred for 1 hour. After the solvent was removed by the distillation under reduced pressure and solidified, the title compound (1.0 g, 77%) was obtained by cleanse the solid with diethyl ether.

MS (M+1): 101.2

### Preparation Example 8-4-6

### {(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-carbamic acid tert-butyl ester

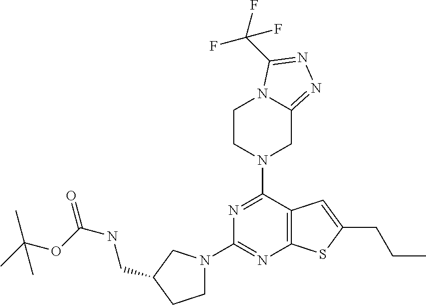

According to the same method to that described in Example 1-1, the reaction using the compound (1.2 g, 2.96 mmol) obtained from Preparation Example 1-1-3, the compound (1 g, 4.43 mmol) obtained from Preparation Example 8-4-5 and diisobutylamine (2.6 mL, 14.78 mmol) was carried out. After the reaction mixture was distilled under reduced pressure to remove the solvent, di t-butyl dicarbonate (1.29 g, 5.91 mmol) was added thereto, diluted with dichloromethane 30 mL and stirred for 2 hours at room temperature. The reaction mixture was distilled under reduced pressure, diluted with ethyl acetate and washed with water and brine. After the organic layer was dried with anhydrous magnesium sulfate and distilled under reduced pressure, the title compound (250 mg, step 2: 15%) was obtained by column-chromatography using 4:1 mixture of dichloromethane and ethyl acetate.

¹H NMR (400 MHz, DMSO); δ 6.76 (1H, s), 5.18 (2H, s), 4.68 (1H, br), 4.35 (2H, t), 4.19 (2H, t), 3.74 (2H, m), 3.55 (1H, m), 3.27 (2H, m), 3.19 (1H, m), 2.79 (2H, t), 2.50 (1H, m), 2.10 (1H, m), 1.75 (2H, m), 1.45 (9H, s), 1.01 (3H, t)

### Preparation Example 8-4-7

### Hydrochloric acid salt of C-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-methylamine

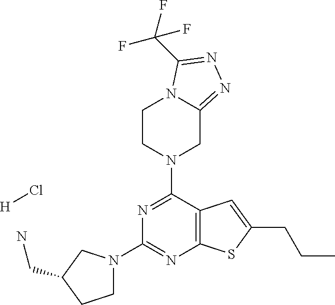

The compound (250 mg, 0.441 mmol) obtained from Preparation Example 8-4-6 was diluted with dichloromethane 30 mL. 4.0 M hydrochloric acid dioxane solution 4 mL was added thereto and stirred for 1 hour. After the solvent was removed by distillation under reduced pressure and solidified, the title compound (235 mg, 99%) was obtained by cleanse the solid with diethyl ether.

MS (M+1): 467.4

### Example 8-4

### N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-acetamide

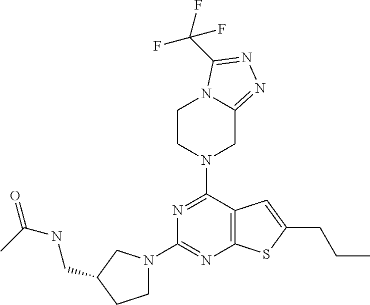

According to the same method to that in Example 1-105, the title compound (20 mg, 24%) was obtained by using the compound (80 mg, 0.165 mmol) obtained from Preparation Example 8-4-7 and acetic anhydride (0.016 mL, 0.165 mmol).

¹H NMR (400 MHz, CDCl₃); δ 6.82 (1H, s), 6.30 (1H, br), 5.14 (2H, s), 4.35 (2H, t), 4.20 (2H, t), 3.76 (2H, m), 3.53 (1H, m), 3.43 (1H, m), 3.29 (2H, m), 2.78 (2H, t), 2.58 (1H, m), 2.18 (1H, m), 2.02 (3H, s), 1.77 (3H, m), 0.99 (3H, t)

### Example 8-5

### N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-butyramide

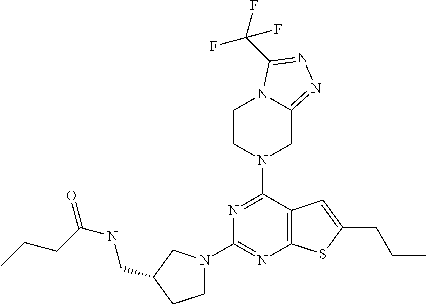

According to the same method to that in Example 1-105, the title compound (25 mg, 28%) was obtained by using the compound (80 mg, 0.165 mmol) obtained from Preparation Example 8-4-7 and butyric anhydride (0.027 mL, 0.165 mmol).

¹H NMR (400 MHz, CDCl₃); δ 6.78 (1H, s), 5.92 (1H, br), 5.17 (2H, s), 4.36 (2H, t), 4.22 (2H, t), 3.78 (2H, m), 3.57 (2H, m), 3.31 (2H, m), 2.80 (2H, t), 2.58 (1H, m), 2.22 (2H, m), 2.14 (1H, m), 1.79 (5H, m), 1.03 (6H, m)

### Example 8-6

### N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylmethyl}-isobutyramide

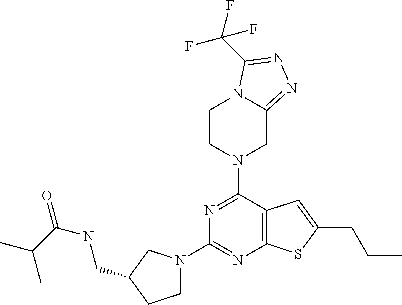

According to the same method to that in Example 1-86, the title compound (30 mg, 34%) was obtained by using the compound (80 mg, 0.165 mmol) obtained from Preparation Example 8-4-7 and isobutyric anhydride (0.018 mL, 0.198 mmol).

¹H NMR (400 MHz, CDCl₃); δ 6.78 (1H, s), 5.88 (1H, br), 5.17 (2H, s), 4.36 (2H, t), 4.26 (2H, t), 3.78 (2H, m), 3.57 (2H, m), 3.31 (2H, m), 2.82 (2H, t), 2.60 (1H, m), 2.45 (1H, m), 2.16 (1H, m), 1.79 (3H, m), 1.20 (6H, d), 1.03 (3H, t)

### Preparation Example 8-7-1

### {2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-carbamic acid tert-butyl ester

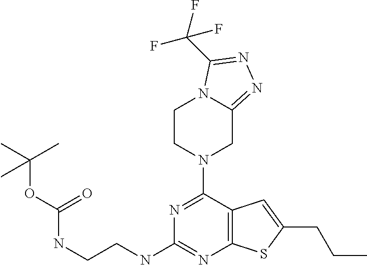

According to the same method to that in Example 1-45, the title compound (650 mg, 16%) was obtained by using the compound (2.13 g, 5.29 mmol) obtained from Preparation Example 1-1-3 and (2-amino-ethyl)-carbamic acid t-butyl ester (3.02 g, 7.49 mmol).

¹H NMR (400 MHz, CDCl₃); δ 6.79 (1H, s), 5.19 (2H, s), 4.33 (2H, t), 4.22 (2H, t), 3.51 (2H, t), 3.34 (2H, t), 2.76 (2H, t), 1.69 (2H, m), 1.42 (9H, s), 0.98 (3H, t)

### Preparation Example 8-7-2

### Hydrochloric acid salt of N-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine

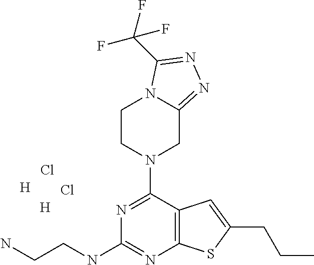

The compound (650 mg, 1.234 mmol) obtained from Preparation Example 8-7-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (580 mg, 94%) was obtained by cleanse the solid with diethyl ether.

¹H NMR (500 MHz, DMSO); δ 8.17 (3H, br), 7.38 (1H, s), 5.22 (2H, s), 4.37 (4H, br), 3.57 (2H, br), 3.00 (2H, br), 2.80 (2H, t), 1.64 (2H, m), 0.93 (3H, t)

### Example 8-7

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-acetamide

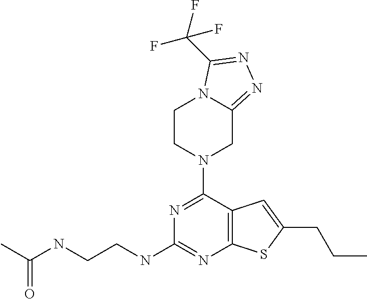

According to the same method to that in Example 1-105, the title compound (55 mg, 62%) was obtained by using the compound (95 mg, 0.190 mmol) obtained from Preparation Example 8-7-2 and acetic anhydride (0.036 mL, 0.380 mmol).

¹H NMR (400 MHz, CDCl₃); δ 6.82 (1H, s), 5.24 (1H, br), 5.20 (2H, s), 4.32 (2H, t), 4.22 (2H, t), 3.54 (2H, t), 3.43 (2H, t), 2.77 (2H, t), 1.97 (3H, s), 1.68 (2H, m), 0.98 (3H, t)

### Example 8-8

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-butyramide

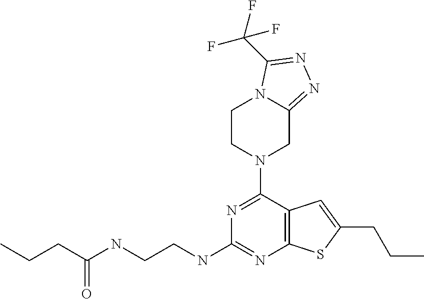

According to the same method to that in Example 1-105, the title compound (63 mg, 67%) was obtained by using the compound (95 mg, 0.190 mmol) obtained from Preparation Example 8-7-2 and butyric anhydride (0.062 mL, 0.380 mmol).

¹H NMR (400 MHz, CDCl₃); δ 6.82 (1H, s), 5.24 (1H, br), 5.20 (2H, s), 4.32 (2H, t), 4.22 (2H, t), 3.56 (2H, t), 3.45 (2H, t), 2.77 (2H, t), 2.12 (2H, t), 1.70 (2H, m), 1.60 (2H, m), 0.98 (3H, t), 0.88 (3H, t)

### Example 8-9

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-isobutyramide

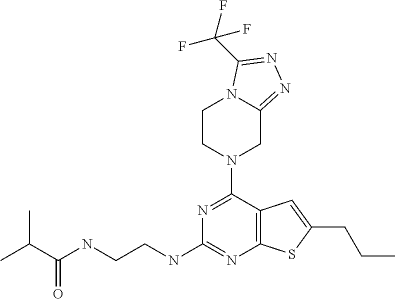

According to the same method to that in Example 1-86, the title compound (70 mg, 74%) was obtained by using the compound (95 mg, 0.190 mmol) obtained from Preparation Example 8-7-2 and isobutyric anhydride (0.021 mL, 0.228 mmol).

¹H NMR (400 MHz, CDCl₃); δ 6.81 (1H, s), 5.20 (2H, s), 4.32 (2H, t), 4.21 (2H, t), 3.55 (2H, t), 3.45 (2H, t), 2.77 (2H, t), 2.30 (1H, m), 1.70 (2H, m), 1.11 (6H, d), 0.98 (3H, t)

### Example 8-10

### 1-{3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-ethanone

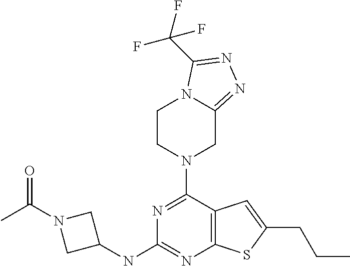

According to the same method to that in Example 1-105, the title compound (45 mg, 49%) was obtained by using the compound (97 mg, 0.190 mmol) obtained from Example 1-83 and acetic anhydride (0.036 mL, 0.380 mmol).

¹H NMR (500 MHz, CDCl₃); δ 6.82 (1H, s), 5.19 (2H, s), 4.70 (1H, m), 4.48 (1H, t), 4.39 (1H, t), 4.31 (2H, t), 4.20 (2H, t), 3.97 (1H, m), 3.90 (1H, m), 2.77 (2H, t), 1.90 (3H, s), 1.69 (2H, m), 0.98 (3H, t)

### Example 8-11

### 1-{3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-butan-1-one

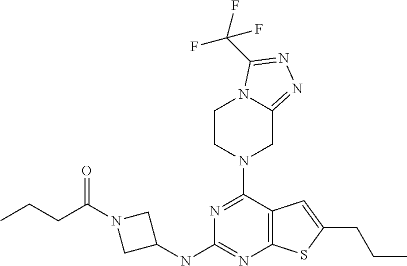

According to the same method to that in Example 1-105, the title compound (61 mg, 63%) was obtained by using the compound (97 mg, 0.190 mmol) obtained from Example 1-83 and butyric anhydride (0.062 mL, 0.380 mmol).

¹H NMR (500 MHz, CDCl₃); δ 6.82 (1H, s), 5.18 (2H, s), 4.68 (1H, m), 4.48 (1H, t), 4.3d7 (1H, t), 4.31 (2H, t), 4.20 (2H, t), 3.95 (1H, m), 3.89 (1H, m), 2.77 (2H, t), 2.07 (2H, t), 1.71 (2H, m), 1.65 (2H, m), 0.99 (3H, t), 0.95 (3H, t)

### Example 8-12

### 2-Methyl-1-{3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-azetidin-1-yl}-propan-1-one

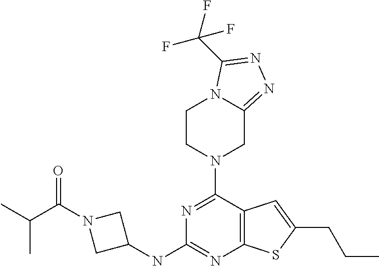

According to the same method to that in Example 1-86, the title compound (2 mg, 10%) was obtained by using the compound (20 mg, 0.039 mmol) obtained from Example 1-83 and isobutyric anhydride (0.004 mL, 0.047 mmol).

¹H NMR (400 MHz, CDCl₃); δ 6.82 (1H, s), 5.23 (1H, t), 5.19 (2H, d), 4.71 (1H, m), 4.50 (1H, t), 4.38 (1H, m), 4.32 (2H, t), 4.21 (2H, t), 3.97 (1H, m), 3.87 (1H, m), 2.77 (2H, t), 2.43 (1H, m), 1.70 (2H, m), 1.10 (6H, d), 0.98 (3H, t)

### Example 8-13

### 1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-ethanone

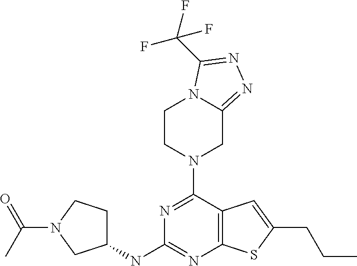

According to the same method to that in Example 1-105, the title compound (95 mg, 47%) was obtained by using the compound (200 mg, 0.409 mmol) obtained from Example 1-126 and acetic anhydride (0.043 mL, 0.45 mmol).

¹H NMR (400 MHz, DMSO); δ 7.21 (1H, s), 7.19 (1H, br), 5.13 (2H, s), 4.50 (1H, m), 4.37 (2H, br), 4.21 (2H, br), 3.77 (1H, m), 3.63 (1H, m), 3.53 (1H, m), 3.36 (1H, m), 2.80 (2H, t), 2.21 (1H, m), 1.95 (4H, m), 1.71 (2H, m), 0.98 (3H, t)

### Example 8-14

### 1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-butan-1-one

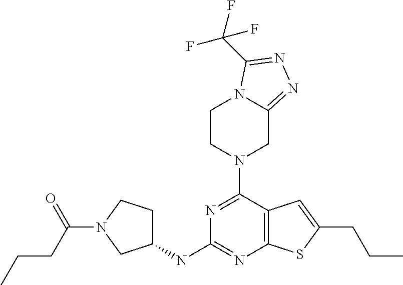

According to the same method to that in Example 1-105, the title compound (83 mg, 39%) was obtained by using the compound (200 mg, 0.409 mmol) obtained from Example 1-126 and butyric anhydride (0.074 mL, 0.45 mmol).

¹H NMR (400 MHz, DMSO); δ 7.21 (1H, s), 7.19 (1H, br), 5.14 (2H, s), 4.45 (1H, m), 4.37 (2H, br), 4.22 (2H, br), 3.76 (1H, m), 3.63 (1H, m), 3.49 (1H, m), 3.32 (1H, m), 2.80 (2H, t), 2.24 (3H, t), 1.96 (1H, m), 1.71 (2H, m), 1.56 (2H, m), 0.98 (3H, t), 0.89 (3H, t)

### Example 8-15

### 2-Methyl-1-{(S)-3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-propan-1-one

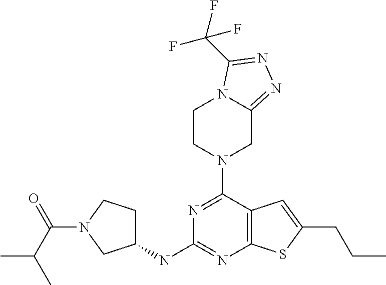

According to the same method to that in Example 1-86, the title compound (55 mg, 26%) was obtained by using the compound (200 mg, 0.409 mmol) obtained from Example 1-126 and isobutyric acid (0.046 mL, 0.491 mmol).

¹H NMR (400 MHz, DMSO); δ 7.21 (1H, s), 7.18 (1H, br), 5.12 (2H, s), 4.45 (1H, m), 4.36 (2H, t), 4.21 (2H, t), 3.81 (1H, m), 3.69 (2H, m), 3.26 (1H, m), 3.32 (1H, m), 2.79 (2H, t), 2.69 (1H, m), 2.21 (1H, m), 1.97 (1H, m), 1.70 (2H, m), 1.01 (3H, t), 0.96 (6H, d)

### Example 8-16

### 1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-ethanone

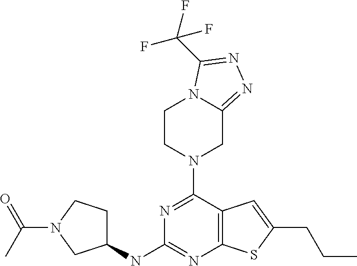

According to the same method to that in Example 1-105, the title compound (10 mg, 20%) was obtained by using the compound (50 mg, 0.102 mmol) obtained from Example 1-130 and acetic anhydride (0.01 mL, 0.112 mmol).

¹H NMR (400 MHz, DMSO); δ 7.21 (1H, s), 7.19 (1H, br), 5.13 (2H, s), 4.50 (1H, m), 4.37 (2H, br), 4.21 (2H, br), 3.77 (1H, m), 3.63 (1H, m), 3.53 (1H, m), 3.36 (1H, m), 2.80 (2H, t), 2.21 (1H, m), 1.95 (4H, m), 1.71 (2H, m), 0.98 (3H, t)

### Example 8-17

### 1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-butan-1-one

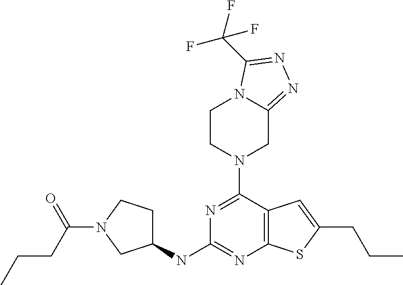

According to the same method to that in Example 1-105, the title compound (27 mg, 51%) was obtained by using the compound (50 mg, 0.102 mmol) obtained from Example 1-130 and butyric anhydride (0.02 mL, 0.112 mmol).

¹H NMR (400 MHz, DMSO); δ 7.21 (1H, s), 7.19 (1H, br), 5.14 (2H, s), 4.45 (1H, m), 4.37 (2H, br), 4.22 (2H, br), 3.76 (1H, m), 3.63 (1H, m), 3.49 (1H, m), 3.32 (1H, m), 2.80 (2H, t), 2.24 (3H, t), 1.96 (1H, m), 1.71 (2H, m), 1.56 (2H, m), 0.98 (3H, t), 0.89 (3H, t)

### Example 8-18

### 2-Methyl-1-{(R)-3-[6-propyl-4-(3-trifluoromethyl-5,6-dihydro-8H-[1,2,4]triazolo[4,3-a]pyrazin-7-yl)-thieno[2,3-d]pyrimidin-2-ylamino]-pyrrolidin-1-yl}-propan-1-one

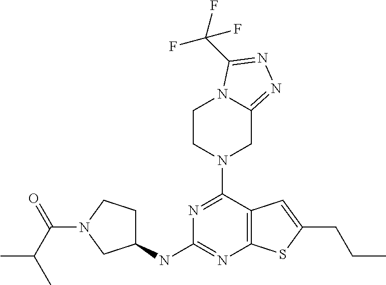

According to the same method to that in Example 1-86, the title compound (21 mg, 39%) was obtained by using the compound (50 mg, 0.102 mmol) obtained from Example 1-130 and isobutyric acid (0.012 mL, 0.123 mmol).

¹H NMR (400 MHz, DMSO); δ 7.21 (1H, s), 7.18 (1H, br), 5.12 (2H, s), 4.45 (1H, m), 4.36 (2H, t), 4.21 (2H, t), 3.81 (1H, m), 3.69 (2H, m), 3.26 (1H, m), 3.32 (1H, m), 2.79 (2H, t), 2.69 (1H, m), 2.21 (1H, m), 1.97 (1H, m), 1.70 (2H, m), 1.01 (3H, t), 0.96 (6H, d)

### Preparation Example 8-19-1

### (S)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidine-1-carboxylic acid tert-butyl ester

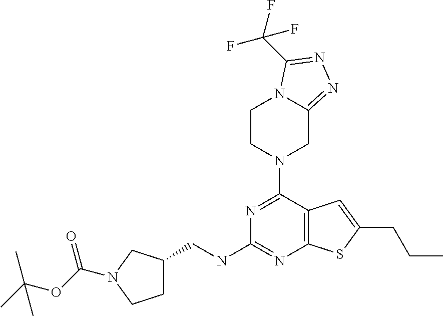

According to the same method to that in Example 1-45, the title compound (200 mg, 13%) was obtained by using the compound (1.1 g, 2.72 mmol) obtained from Preparation Example 1-1-3 and the compound (0.6 g, 3.00 mmol) obtained from Preparation Example 8-1-4.

¹H NMR (400 MHz, DMSO); δ 6.79 (1H, s), 5.18 (2H, s), 4.95 (1H, br), 4.35 (2H, t), 4.21 (2H, t), 3.46 (4H, m), 3.14˜3.33 (2H, m), 2.80 (2H, t), 2.51 (1H, m), 2.00 (1H, br), 1.77 (3H, m), 1.46 (9H, s), 1.02 (3H, t)

### Preparation Example 8-19-2

### Hydrochloric acid salt of

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(R)-1-pyrrolidin-3-ylmethyl-amine

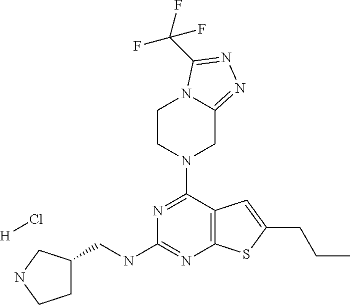

The compound (200 mg, 0.353 mmol) obtained from Preparation Example 8-19-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (150 mg, 85%) was obtained by cleanse the solid with diethyl ether.

MS (M+1): 467.4

### Example 8-19

### 1-((S)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-ethanone

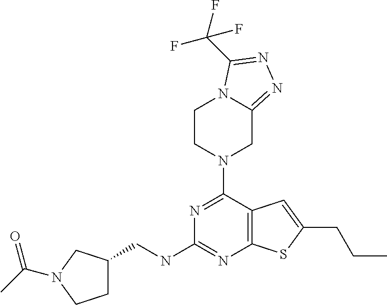

According to the same method to that in Example 1-105, the title compound (18 mg, 32%) was obtained by using the compound (50 mg, 0.111 mmol) obtained from Preparation Example 8-19-2 and acetic anhydride (0.01 mL, 0.111 mmol).

¹H NMR (400 MHz, CDCl₃); δ 6.82 (1H, s), 5.19 (2H, s), 4.36 (2H, t), 4.24 (2H, t), 3.70 (2H, m), 3.41˜3.57 (3H, m), 3.28 (1H, m), 2.80 (2H, t), 2.65 (1H, m), 2.19 (1H, m), 2.04 (3H, d), 1.84 (1H, m), 1.73 (2H, m), 1.32 (1H, m), 1.00 (3H, t)

### Example 8-20

### 1-((S)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-butan-1-one

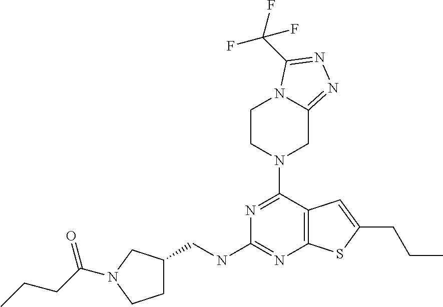

According to the same method to that in Example 1-105, the title compound (20 mg, 33%) was obtained by using the compound (50 mg, 0.111 mmol) obtained from Preparation Example 8-19-2 and butyric anhydride (0.02 mL, 0.111 mmol).

¹H NMR (400 MHz, CDCl₃); δ 6.83 (1H, s), 5.21 (2H, s), 4.38 (2H, t), 4.25 (2H, t), 3.74 (2H, m), 3.44˜3.53 (3H, m), 3.24˜3.29 (1H, m), 2.82 (2H, t), 2.51˜2.65 (1H, m), 2.28 (2H, q), 2.07˜2.17 (1H, m), 1.64˜1.85 (5H, m), 1.04 (6H, m)

### Example 8-21

### 2-Methyl-1-((S)-3-{[6-propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-propan-1-one

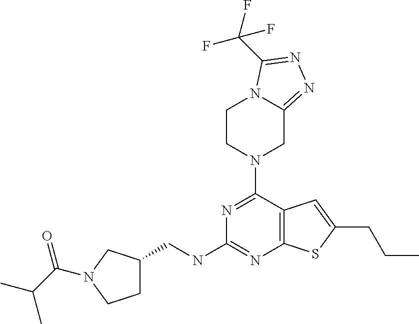

According to the same method to that in Example 1-86, the title compound (20 mg, 33%) was obtained by using the compound (50 mg, 0.111 mmol) obtained from Preparation Example 8-19-2 and isobutyric acid (0.012 mL, 0.133 mmol).

¹H NMR (400 MHz, CDCl₃); δ 6.80 (1H, s), 5.18 (2H, s), 4.35 (2H, t), 4.21 (2H, t), 3.69 (2H, m), 3.45˜3.52 (2H, m), 3.31 (1H, m), 2.80 (2H, t), 2.50˜2.65 (2H, m), 2.15 (1H, m), 1.66˜1.83 (4H, m), 1.13 (6H, d), 1.00 (3H, t)

### Preparation Example 8-22-1

### (R)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidine-1-carboxylic acid tert-butyl ester

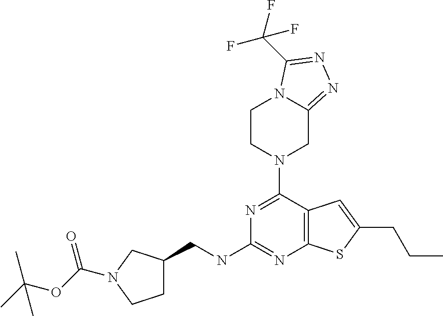

According to the same method to that in Example 1-45, the title compound (700 mg, 30%) was obtained by using the compound (1.68 g, 4.16 mmol) obtained from Preparation Example 1-1-3 and the compound (1.0 g, 4.993 mmol) obtained from Preparation Example 8-4-4

¹H NMR (400 MHz, DMSO); δ 6.79 (1H, s), 5.18 (2H, s), 4.95 (1H, br), 4.35 (2H, t), 4.21 (2H, t), 3.46 (4H, m), 3.14˜3.33 (2H, m), 2.80 (2H, t), 2.51 (1H, m), 2.00 (1H, br), 1.77 (3H, m), 1.46 (9H, s), 1.02 (3H, t)

### Preparation Example 8-22-2

### Hydrochloric acid salt of

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(S)-1-pyrrolidin-3-ylmethyl-amine

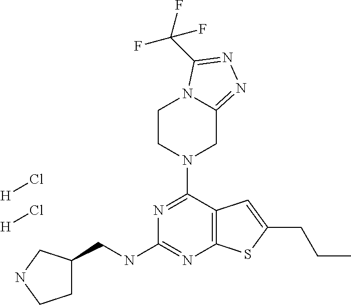

The compound (700 mg, 1.235 mmol) obtained from Preparation Example 8-22-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (600 mg, 97%) was obtained by cleanse the solid with diethyl ether.

MS (M+1): 467.4

### Example 8-22

### 1-((R)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-ethanone

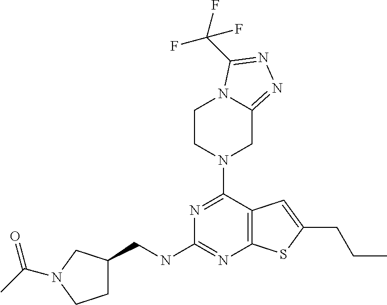

According to the same method to that in Example 1-105, the title compound (35 mg, 31%) was obtained by using the compound (100 mg, 0.222 mmol) obtained from Preparation Example 8-22-2 and acetic anhydride (0.02 mL, 0.222 mmol).

¹H NMR (400 MHz, CDCl₃); δ 6.82 (1H, s), 5.19 (2H, s), 4.36 (2H, t), 4.24 (2H, t), 3.70 (2H, m), 3.41˜3.57 (3H, m), 3.28 (1H, m), 2.80 (2H, t), 2.65 (1H, m), 2.19 (1H, m), 2.04 (3H, d), 1.84 (1H, m), 1.73 (2H, m), 1.32 (1H, m), 1.00 (3H, t)

### Example 8-23

### 1-((R)-3-{[6-Propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-butan-1-one

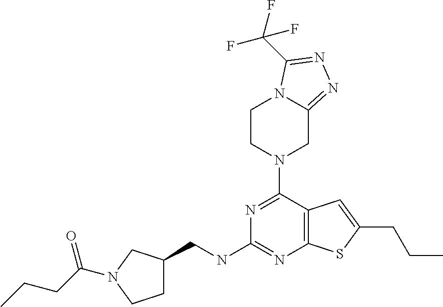

According to the same method to that in Example 1-105, the title compound (40 mg, 33%) was obtained by using the compound (100 mg, 0.222 mmol) obtained from Preparation Example 8-22-2 and butyric anhydride (0.04 mL, 0.222 mmol).

¹H NMR (400 MHz, CDCl₃); δ 6.83 (1H, s), 5.21 (2H, s), 4.38 (2H, t), 4.25 (2H, t), 3.74 (2H, m), 3.44˜3.53 (3H, m), 3.24˜3.29 (1H, m), 2.82 (2H, t), 2.51˜2.65 (1H, m), 2.28 (2H, q), 2.07˜2.17 (1H, m), 1.64˜1.85 (5H, m), 1.04 (6H, m)

### Example 8-24

### 2-Methyl-1-((R)-3-{[6-propyl-4-(3-trifluoromethyl-5,6-dihydro-8H-[1,2,4]triazolo[4,3-a]pyrazin-7-yl)-thieno[2,3-d]pyrimidin-2-ylamino]-methyl}-pyrrolidin-1-yl)-propan-1-one

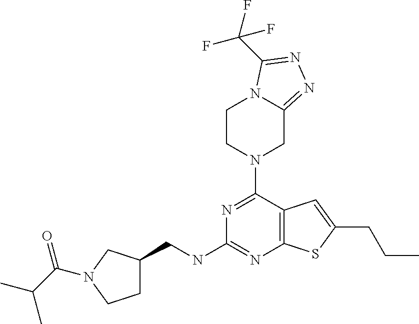

According to the same method to that in Example 1-86, the title compound (37 mg, 31%) was obtained by using the compound (100 mg, 0.223 mmol) obtained from Preparation Example 8-22-2 and isobutyric acid (0.025 mL, 0.267 mmol).

¹H NMR (400 MHz, CDCl₃); δ 6.80 (1H, s), 5.18 (2H, s), 4.35 (2H, t), 4.21 (2H, t), 3.69 (2H, m), 3.45˜3.52 (2H, m), 3.31 (1H, m), 2.80 (2H, t), 2.50-2.65 (2H, m), 2.15 (1H, m), 1.66˜1.83 (4H, m), 1.13 (6H, d), 1.00 (3H, t)

### Example 8-25

### 1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-ethanone

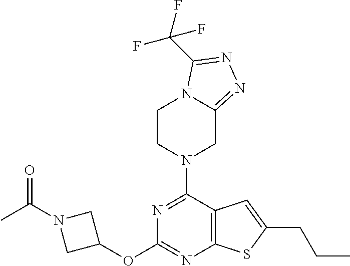

According to the same method to that in Example 1-105, the title compound (40 mg, 44%) was obtained by using the compound (90 mg, 0.190 mmol) obtained from Example 3-26 and acetic anhydride (0.036 mL, 0.380 mmol).

¹H NMR (400 MHz, CDCl₃); δ 6.96 (1H, s), 5.38 (1H, m), 5.30 (2H, d), 4.53 (1H, m), 4.31˜4.35 (5H, m), 4.22 (1H, m), 4.12 (1H, m), 2.83 (2H, t), 1.91 (3H, s), 1.73 (2H, m), 1.00 (3H, t)

### Example 8-26

### 1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-butan-1-one

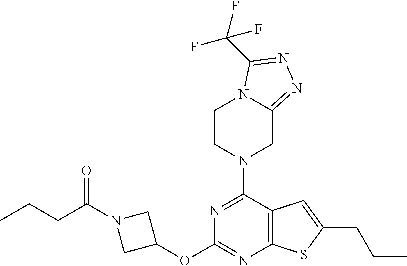

According to the same method to that in Example 1-105, the title compound (50 mg, 52%) was obtained by using the compound (90 mg, 0.190 mmol) obtained from Example 3-26 and butyric anhydride (0.062 mL, 0.380 mmol).

¹H NMR (400 MHz, CDCl₃); δ 6.96 (1H, s), 5.38 (1H, m), 5.31 (2H, d), 4.53 (1H, m), 4.31˜4.42 (5H, m), 4.23 (1H, m), 4.14 (1H, m), 2.83 (2H, t), 2.08 (2H, t), 1.73 (2H, m), 1.63 (2H, m), 1.00 (3H, t), 0.94 (3H, t)

### Example 8-27

### 2-Methyl-1-{3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-azetidin-1-yl}-propan-1-one

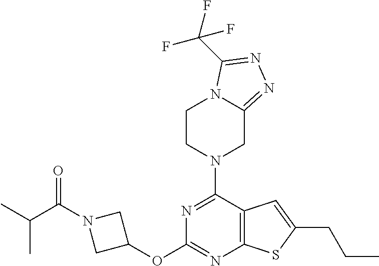

According to the same method to that in Example 1-86, the title compound (65 mg, 67%) was obtained by using the compound (90 mg, 0.190 mmol) obtained from Example 3-26 and isobutyric acid (0.021 mL, 0.228 mmol).

¹H NMR (400 MHz, CDCl₃); δ 6.96 (1H, s), 5.39 (1H, m), 5.31 (2H, d), 4.54 (1H, m), 4.31˜4.44 (5H, m), 4.26 (1H, m), 4.09 (1H, m), 2.83 (2H, t), 2.43 (1H, m), 1.73 (2H, m), 1.10 (6H, d), 1.00 (3H, t)

### Preparation Example 8-28-1

### (R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

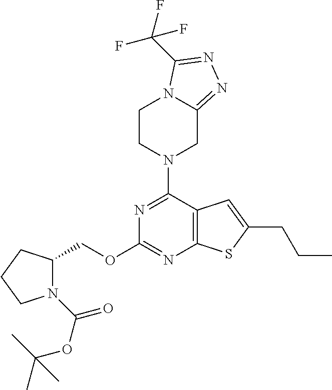

According to the same method to that in Example 1-45, the title compound (424 mg, 14%) was obtained by using the compound (2.23 g, 5.54 mmol) obtained from Preparation Example 1-1-3 and (R)-2-hydroxymethyl-pyrrolidine-1-carboxylic acid t-butyl ester (2.23 g, 11.08 mmol).

¹H NMR (400 MHz, CDCl₃); δ 6.94 (1H, s), 5.32 (2H, s), 4.39 (4H, br), 4.22 (3H, br), 3.41 (2H, br), 2.81 (2H, t), 1.74˜1.96 (4H, br), 1.72 (2H, m), 1.46 (9H, s), 0.99 (3H, t)

### Preparation Example 8-28-2

### Hydrochloride acid salt of

### 7-[6-Propyl-2-((R)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine

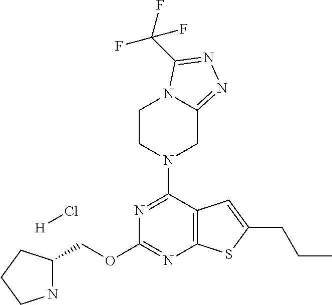

The compound (424 mg, 0.747 mmol) obtained from Preparation Example 8-28-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (370 mg, 98%) was obtained by cleanse the solid with diethyl ether.

¹H NMR (400 MHz, DMSO); δ 9.40 (1H, br), 8.93 (1H, br), 7.42 (1H, s), 5.21 (2H, s), 4.53 (1H, m), 4.40 (1H, m), 4.33 (2H, t), 4.28 (2H, t), 3.89 (1H, br), 3.17 (2H, br), 2.80 (2H, t), 2.09 (1H, m), 1.88 (2H, m), 1.64 (3H, m), 0.91 (3H, t)

### Example 8-28

### -{(R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone

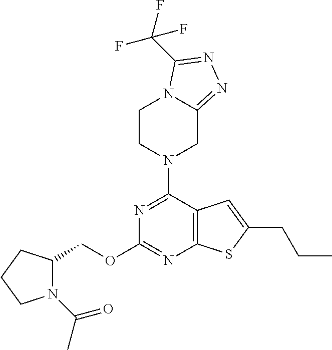

According to the same method to that in Example 1-105, the title compound (50 mg, 52%) was obtained by using the compound (96 mg, 0.190 mmol) obtained from Preparation Example 8-28-2 and acetic anhydride (0.036 mL, 0.380 mmol).

¹H NMR (400 MHz, CDCl₃); δ 6.94 (1H, s), 5.31 (2H, s), 4.62 (1H, m), 4.26˜4.50 (6H, m), 3.44˜3.57 (2H, m), 2.80 (2H, t), 2.12 (2H, m), 2.06 (3H, s), 1.97 (1H, br), 1.70 (3H, m), 0.99 (3H, t)

### Example 8-29

### 1-{(R)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one

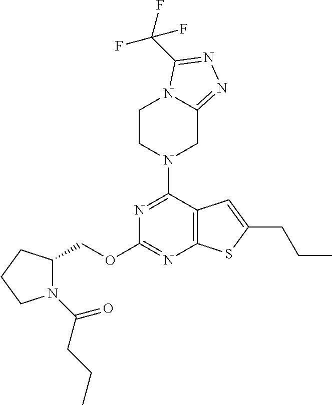

According to the same method to that in Example 1-105, the title compound (30 mg, 29%) was obtained by using the compound (96 mg, 0.190 mmol) obtained from Preparation Example 8-28-2 and butyric anhydride (0.062 mL, 0.380 mmol).

¹H NMR (400 MHz, CDCl₃); δ 6.94 (1H, s), 5.32 (2H, s), 4.64 (1H, m), 4.24˜4.51 (6H, m), 3.45˜3.54 (2H, m), 2.80 (2H, t), 2.49 (0.4H, t), 2.22 (1.6H, t), 2.12 (2H, br), 1.98 (2H, br), 1.70 (2H, m), 1.62 (2H, m), 1.00 (3H, t), 0.93 (3H, t)

### Example 8-30

### 2-Methyl-1-{(R)-2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one

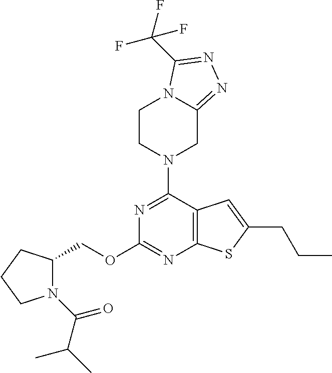

According to the same method to that in Example 1-86, the title compound (60 mg, 59%) was obtained by using the compound (96 mg, 0.190 mmol) obtained from Preparation Example 8-28-2 and isobutyric acid (0.021 mL, 0.228 mmol).

¹H NMR (400 MHz, CDCl₃); δ 6.93 (1H, s), 5.31 (2H, s), 4.60 (1H, m), 4.25˜4.50 (6H, m), 3.48˜3.59 (2H, m), 2.80 (2H, t), 2.62 (1H, t), 2.12 (1.7H, m), 1.99 (2.3H, br), 1.72 (2H, m), 1.06 (6H, m) 0.99 (3H, t)

### Preparation Example 8-31-1

### (S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

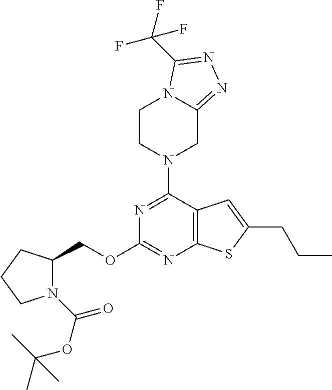

According to the same method to that in Example 1-45, the title compound (1.2 g, 40%) was obtained by using the compound (2.13 g, 5.29 mmol) obtained from Preparation Example 1-1-3 and (S)-2-hydroxymethyl-pyrrolidine-1-carboxylic acid t-butyl ester (2.13 g, 10.59 mmol).

¹H NMR (500 MHz, CDCl₃); δ 6.94 (1H, s), 5.32 (2H, s), 4.39 (4H, br), 4.22 (3H, br), 3.41 (2H, br), 2.81 (2H, t), 1.74˜1.96 (4H, br), 1.72 (2H, m), 1.46 (9H, s), 0.99 (3H, t)

### Preparation Example 8-31-2

### Hydrochloric acid salt of

### 7-[6-Propyl-2-((S)-1-pyrrolidin-2-ylmethoxy)-thieno[2,3-d]pyrimidin-4-yl]-3-trifluoromethyl-5,6,7,8-tetrahydro-[1,2,4]triazolo[4,3-a]pyrazine

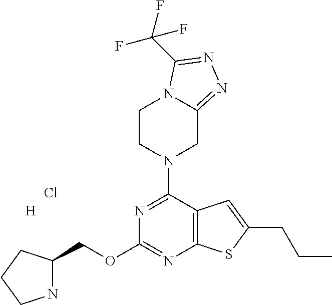

The compound (400 mg, 0.705 mmol) obtained from Preparation Example 8-31-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (355 mg, 100%) was obtained by cleanse the solid with diethyl ether.

### Example 8-31

### 1-{(S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone

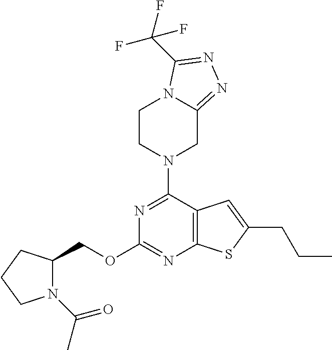

According to the same method to that in Example 1-105, the title compound (46 mg, 47%) was obtained by using the compound (96 mg, 0.190 mmol) obtained from Preparation Example 8-31-2 and acetic anhydride (0.036 mL, 0.380 mmol).

¹H NMR (400 MHz, CDCl₃); δ 6.94 (1H, s), 5.31 (2H, s), 4.62 (1H, m), 4.26˜4.50 (6H, m), 3.44˜3.57 (2H, m), 2.80 (2H, t), 2.12 (2H, m), 2.06 (3H, s), 1.97 (1H, br), 1.70 (3H, m), 0.99 (3H, t)

### Example 8-32

### 1-{(S)-2-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one

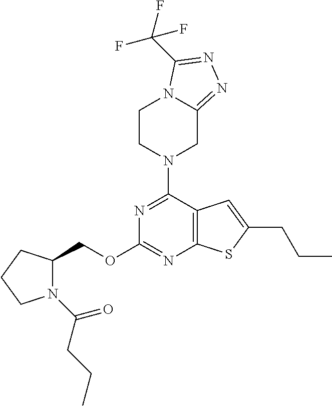

According to the same method to that in Example 1-105, the title compound (70 mg, 68%) was obtained by using the compound (96 mg, 0.190 mmol) obtained from Preparation Example 8-31-2 and butyric anhydride (0.062 mL, 0.380 mmol).

¹H NMR (400 MHz, CDCl₃); δ 6.94 (1H, s), 5.32 (2H, s), 4.64 (1H, m), 4.24˜4.51 (6H, m), 3.45˜3.54 (2H, m), 2.80 (2H, t), 2.49 (0.4H, t), 2.22 (1.6H, t), 2.12 (2H, br), 1.98 (2H, br), 1.70 (2H, m), 1.62 (2H, m), 1.00 (3H, t), 0.93 (3H, t)

### Example 8-33

### 2-Methyl-1-{(S)-2-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one

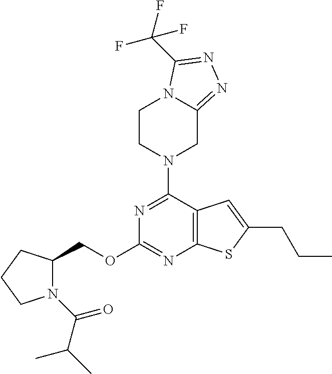

According to the same method to that in Example 1-86, the title compound (50 mg, 49%) was obtained by using the compound (96 mg, 0.190 mmol) obtained from Preparation Example 8-31-2 and isobutyric acid (0.021 mL, 0.380 mmol).

¹H NMR (400 MHz, CDCl₃); δ 6.93 (1H, s), 5.31 (2H, s), 4.60 (1H, m), 4.25˜4.50 (6H, m), 3.48˜3.59 (2H, m), 2.80 (2H, t), 2.62 (1H, t), 2.12 (1.7H, m), 1.99 (2.3H, br), 1.72 (2H, m), 1.06 (6H, m) 0.99 (3H, t)

### Preparation Example 8-34-1

### (R)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

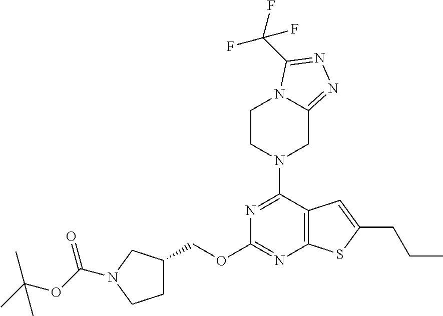

According to the same method to that in Example 1-45, the title compound (600 mg, 26%) was obtained by using the compound (1.64 g, 4.065 mmol) obtained from Preparation Example 1-1-3 and the compound (0.9 g, 4.472 mmol) obtained from Preparation Example 8-1-3.

¹H NMR (500 MHz, DMSO); δ 7.08 (1H, s), 5.34 (2H, s), 4.43 (2H, t), 4.36 (2H, t), 3.67 (2H, m), 3.14˜3.53 (4H, m), 2.88 (2H, t), 2.78 (1H, br), 2.13 (1H, br), 2.00 (1H, br), 1.77 (3H, m), 1.50 (9H, s), 1.07 (3H, t)

### Preparation Example 8-34-2

### Hydrochloric acid salt of

### 7-[6-Propyl-2-((R)-1-pyrrolidin-3-ylmethoxy)-thieno[2,3-d]pyrimidin-4-yl]-3-trifluoromethyl-5,6,7,8-tetrahydro-[1,2,4]triazolo[4,3-a]pyrazine

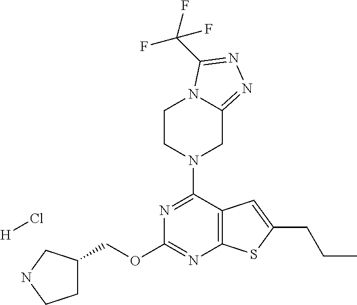

The compound (600 mg, 1.06 mmol) obtained from Preparation Example 8-34-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (500 mg, 94%) was obtained by cleanse the solid with diethyl ether.

MS (M+1): 467.4

### Example 8-34

### 1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone

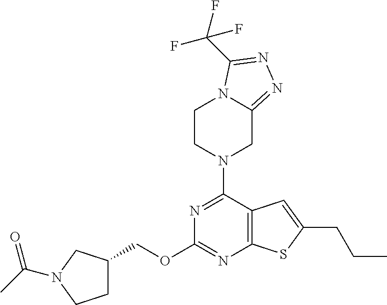

According to the same method to that in Example 1-105, the title compound (45 mg, 40%) was obtained by using the compound (100 mg, 0.222 mmol) obtained from Preparation Example 8-34-2 and acetic anhydride (0.02 mL, 0.222 mmol).

¹H NMR (500 MHz, CDCl₃); δ 6.96 (1H, s), 5.30 (2H, s), 4.26˜4.46 (6H, m), 3.74 (2H, m), 3.36˜3.51 (2H, m), 2.86 (2H, t), 2.80 (1H, m), 2.25 (1H, m), 2.06 (3H, s), 1.98 (1H, m), 1.80 (1H, m), 1.78 (2H, m), 1.03 (3H, t)

### Example 8-35

### 1-{(R)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one

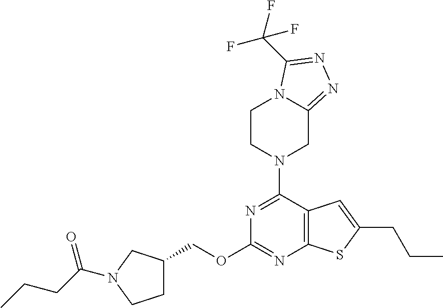

According to the same method to that in Example 1-105, the title compound (32 mg, 27%) was obtained by using the compound (100 mg, 0.222 mmol) obtained from Preparation Example 8-34-2 and butyric anhydride (0.04 mL, 0.222 mmol).

¹H NMR (500 MHz, CDCl₃); δ 6.96 (1H, s), 5.31 (2H, s), 4.23˜4.56 (6H, m), 3.68˜3.77 (2H, m), 3.34˜3.50 (2H, m), 2.86 (2H, t), 2.75 (1H, m), 2.34 (1H, m), 2.23 (2H, t), 1.95˜2.20 (2H, m), 1.62˜1.81 (4H, m), 1.03 (3H, t), 0.96 (3H, m)

### Example 8-36

### 2-Methyl-1-{(R)-3-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one

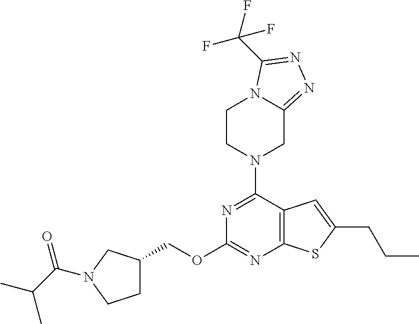

According to the same method to that in Example 1-86, the title compound (80 mg, 67%) was obtained by using the compound (100 mg, 0.222 mmol) obtained from Preparation Example 8-34-2 and isobutyric acid (0.025 mL, 0.267 mmol).

¹H NMR (500 MHz, CDCl₃); δ 6.94 (1H, s), 5.30 (2H, s), 4.25˜4.46 (6H, m), 3.66˜3.80 (2H, m), 3.34˜3.56 (2H, m), 2.85 (2H, t), 2.82 (1H, m), 2.68 (1H, m), 2.09˜2.23 (1H, m), 1.76˜1.98 (1H, m), 1.78 (2H, m), 1.14 (6H, d), 1.03 (3H, t)

### Preparation Example 8-37-1

### (S)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidine-1-carboxylic acid tert-butyl ester

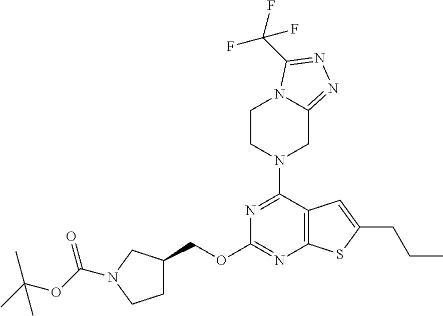

According to the same method to that in Example 1-45, the title compound (500 mg, 39%) was obtained by using the compound (0.91 g, 2.258 mmol) obtained from Preparation Example 1-1-3 and the compound (0.5 g, 2.484 mmol) obtained from Preparation Example 8-4-3.

¹H NMR (500 MHz, DMSO); δ 7.08 (1H, s), 5.34 (2H, s), 4.43 (2H, t), 4.36 (2H, t), 3.67 (2H, m), 3.14˜3.53 (4H, m), 2.88 (2H, t), 2.78 (1H, br), 2.13 (1H, br), 2.00 (1H, br), 1.77 (3H, m), 1.50 (9H, s), 1.07 (3H, t)

### Preparation Example 8-37-2

### Hydrochloric acid salt of

### 7-[6-Propyl-2-((S)-1-pyrrolidin-3-ylmethoxy)-thieno[2,3-d]pyrimidin-4-yl]-3-trifluoromethyl-5,6,7,8-tetrahydro-[1,2,4]triazolo[4,3-a]pyrazine

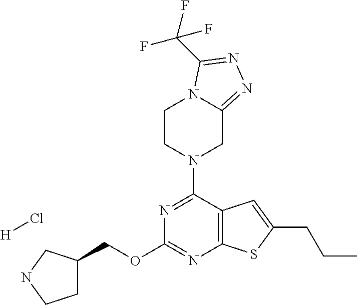

The compound (500 mg, 0.881 mmol) obtained from Preparation Example 8-37-1 was diluted with dichloromethane 30 mL. 4.0 M hydrochloric acid dioxane solution 4 mL was added thereto and stirred for 1 hour. After the solvent was removed by distillation under reduced pressure and solidified, the title compound (420 mg, 95%) was obtained by cleanse the solid with diethyl ether.

MS (M+1): 467.4

### Example 8-37

### 1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-ethanone

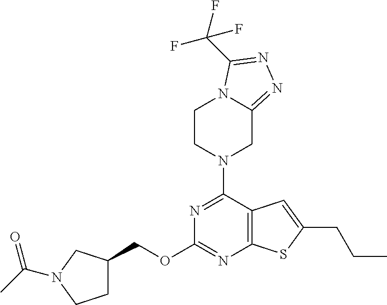

According to the same method to that in Example 1-105, the title compound (30 mg, 27%) was obtained by using the compound (100 mg, 0.222 mmol) obtained from Preparation Example 8-37-2 and acetic anhydride (0.02 mL, 0.222 mmol).

¹H NMR (500 MHz, CDCl₃); δ 6.96 (1H, s), 5.30 (2H, s), 4.26˜4.46 (6H, m), 3.65˜3.77 (2H, m), 3.33˜3.53 (2H, m), 2.86 (2H, t), 2.80 (1H, m), 2.25 (1H, m), 2.06 (3H, s), 1.98 (1H, m), 1.80 (1H, m), 1.78 (2H, m), 1.03 (3H, t)

### Example 8-38

### 1-{(S)-3-[6-Propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-butan-1-one

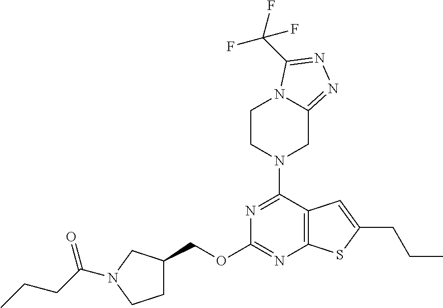

According to the same method to that in Example 1-105, the title compound (35 mg, 29%) was obtained by using the compound (100 mg, 0.222 mmol) obtained from Preparation Example 8-37-2 and butyric anhydride (0.04 mL, 0.222 mmol).

¹H NMR (500 MHz, CDCl₃); δ 6.96 (1H, s), 5.31 (2H, s), 4.23˜4.56 (6H, m), 3.68˜3.77 (2H, m), 3.34˜3.50 (2H, m), 2.86 (2H, t), 2.75 (1H, m), 2.34 (1H, m), 2.23 (2H, t), 1.95˜2.20 (2H, m), 1.62˜1.81 (4H, m), 1.04 (3H, t), 0.96 (3H, m)

### Example 8-39

### 2-Methyl-1-{(S)-3-[6-propyl-4-(3-trifluoromethyl-5,6-dihydro-8H-[1,2,4]triazolo[4,3-a]pyrazin-7-yl)-thieno[2,3-d]pyrimidin-2-yloxymethyl]-pyrrolidin-1-yl}-propan-1-one

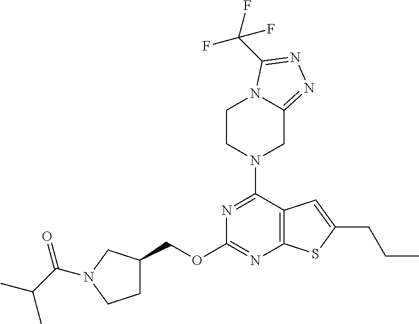

According to the same method to that in Example 1-86, the title compound (40 mg, 34%) was obtained by using the compound (100 mg, 0.222 mmol) obtained from Preparation Example 8-37-2 and isobutyric acid (0.025 mL, 0.267 mmol).

¹H NMR (500 MHz, CDCl₃); δ 6.94 (1H, s), 5.30 (2H, s), 4.25˜4.46 (6H, m), 3.66˜3.80 (2H, m), 3.34˜3.56 (2H, m), 2.85 (2H, t), 2.82 (1H, m), 2.68 (1H, m), 2.09˜2.23 (1H, m), 1.76˜1.98 (1H, m), 1.78 (2H, m), 1.14 (6H, d), 1.03 (3H, t)

### Example 9-1

### {4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid ethyl ester

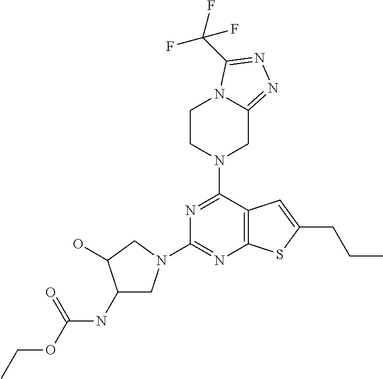

The compound (200 mg, 0.369 mmol) obtained from Example 1-114 was dissolved in dichloromethane 30 mL and cooled to 0° C. Triethylamine (0.15 mL, 1.108 mmol) and ethylchloroformate (0.035 mL, 0.369 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (130 mg, 65.3%) was obtained by column-chromatography using 20:1 mixture of dichloromethane and methanol.

¹H NMR (400 MHz, CDCl₃); δ 7.38 (1H, br), 7.21 (1H, s), 5.29 (1H, d), 5.14 (2H, s), 4.36 (2H, t), 4.20 (2H, t), 4.11 (1H, br), 3.99 (2H, q), 3.89 (1H, br), 3.73 (2H, br), 3.42 (2H, br), 2.76 (2H, t), 1.63 (2H, m), 1.16 (3H, t), 0.94 (3H, t)

### Example 9-2

### {4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester

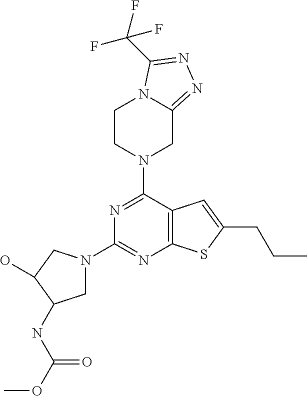

The compound (800 mg, 1.478 mmol) obtained from Example 1-114 was dissolved in dichloromethane 100 mL and cooled to 0° C. Triethylamine (0.62 mL, 4.433 mmol) and methylchloroformate (0.126 mL, 1.625 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (540 mg, 69.4%) was obtained by column-chromatography using 20:1 mixture of dichloromethane and methanol.

¹H NMR (400 MHz, CDCl₃); δ 6.75 (1H, s), 5.52 (1H, br), 5.11 (2H, s), 4.37 (1H, br), 4.27 (2H, t), 4.14 (2H, t), 4.09 (1H, t), 3.91 (1H, q), 3.86 (1H, q), 3.67 (3H, s), 3.49 (2H, m), 2.74 (2H, t), 1.62 (2H, m), 0.93 (3H, t)

### Example 9-3

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid ethyl ester

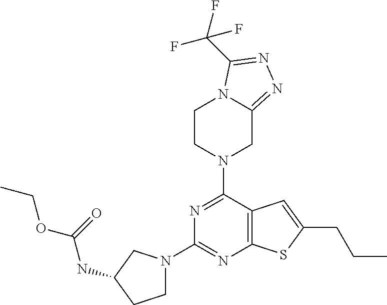

The compound (200 mg, 0.381 mmol) obtained from Example 1-14 was dissolved in dichloromethane 30 mL and cooled to 0° C. Triethylamine (0.16 mL, 1.142 mmol) and ethylchloroformate (0.04 mL, 0.381 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (56 mg, 28%) was obtained by column-chromatography using 20:1 mixture of dichloromethane and methanol.

¹H NMR (400 MHz, CDCl₃); δ 6.78 (1H, s), 5.19 (2H, s), 4.77 (1H, br), 4.36 (1H, br), 4.33 (2H, t), 4.19 (2H, t), 4.12 (2H, m), 3.83 (1H, m), 3.65 (2H, m), 3.48 (1H, q), 2.76 (2H, t), 2.23 (1H, m), 1.94 (1H, m), 1.69 (2H, m), 1.25 (3H, t), 0.98 (3H, t)

### Example 9-4

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid 4-fluoro-phenyl ester

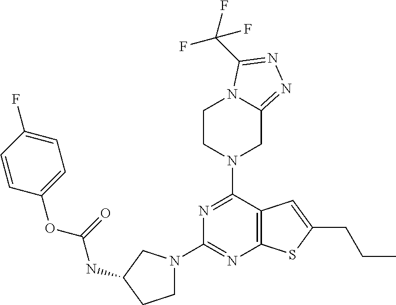

The compound (200 mg, 0.381 mmol) obtained from Example 1-14 was dissolved in dichloromethane 30 mL and cooled to 0° C. Triethylamine (0.16 mL, 1.142 mmol) and 4-fluorophenylchloroformate (0.05 mL, 0.381 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (200 mg, 88.9%) was obtained by column-chromatography using 20:1 mixture of dichloromethane and methanol.

¹H NMR (400 MHz, CDCl₃); δ 7.06˜7.15 (4H, m), 6.83 (1H, s), 5.24 (2H, s), 4.49 (1H, m), 4.37 (2H, t), 4.25 (2H, t), 3.92 (1H, m), 3.74 (2H, m), 3.63 (1H, m), 2.81 (2H, t), 2.35 (1H, m), 2.09 (1H, m), 1.72 (2H, m), 1.02 (3H, t)

### Example 9-5

### Cyclopentanecarboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

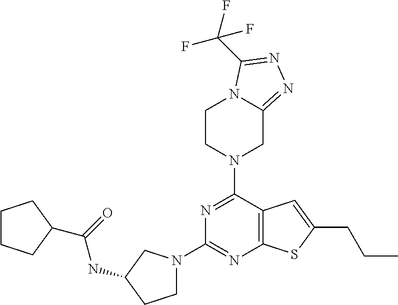

According to the same method to that described in Example 1-86, the title compound (65 mg, 31.1%) was obtained by using the compound (200 mg, 0.381 mmol) obtained from Example 1-14 and cyclopentanecarboxylic acid (0.05 mL, 0.456 mmol).

¹H NMR (400 MHz, CDCl₃); δ 6.82 (1H, s), 5.62 (1H, d), 5.23 (2H, s), 4.64 (1H, m), 4.37 (2H, t), 4.24 (2H, t), 3.88 (1H, m), 3.69 (2H, t), 3.47 (1H, q), 2.80 (2H, t), 2.49 (1H, m), 2.28 (1H, m), 1.97 (1H, m), 1.64˜1.88 (8H, m), 1.61 (2H, m), 1.02 (3H, t)

### Example 9-6

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid ethyl ester

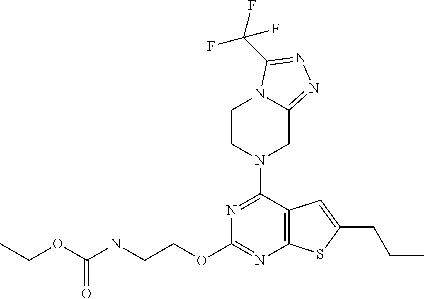

The compound (200 mg, 0.411 mmol) obtained from Example 3-58 was dissolved in dichloromethane 30 mL and cooled to 0° C. Triethylamine (0.17 mL, 1.142 mmol) and ethylchloroformate (0.04 mL, 0.411 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (120 mg, 58.4%) was obtained by column-chromatography using 20:1 mixture of dichloromethane and methanol.

¹H NMR (400 MHz, CDCl₃); δ 6.99 (1H, s), 5.35 (2H, s), 5.20 (1H, br), 4.48 (2H, t), 4.41 (2H, t), 4.36 (2H, t), 4.13 (2H, q), 3.65 (2H, q), 2.86 (2H, t), 1.75 (2H, m), 1.26 (3H, t), 1.04 (3H, t)

### Example 9-7

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid 4-fluoro-phenyl ester

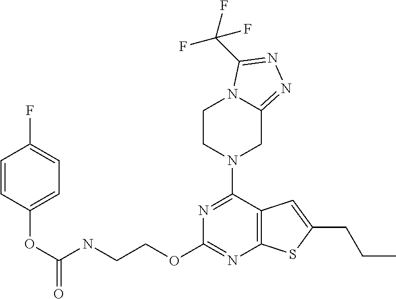

The compound (200 mg, 0.411 mmol) obtained from Example 3-58 was dissolved in dichloromethane 30 mL and cooled to 0° C. Triethylamine (0.17 mL, 1.234 mmol) and 4-fluorophenylchloroformate (0.06 mL, 0.411 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (130 mg, 55.9%) was obtained by column-chromatography using 20:1 mixture of dichloromethane and methanol.

¹H NMR (400 MHz, CDCl₃); δ 6.99 (1H, s), 5.35 (2H, s), 5.20 (1H, br), 4.48 (2H, t), 4.41 (2H, t), 4.36 (2H, t), 4.13 (2H, q), 3.65 (2H, q), 2.86 (2H, t), 1.75 (2H, m), 1.26 (3H, t), 1.04 (3H, t)

### Example 9-8

### Cyclopentanecarboxylic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

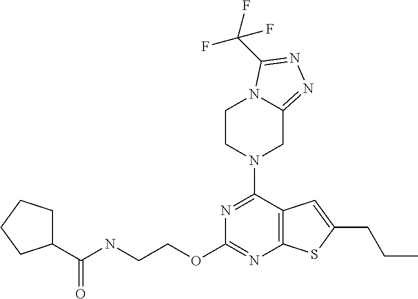

According to the same method to that described in Example 1-86, the title compound (65 mg, 30.2%) was obtained by using the compound (200 mg, 0.411 mmol) obtained from Example 3-58 and cyclopentanecarboxylic acid (0.054 mL, 0.494 mmol).

¹H NMR (400 MHz, CDCl₃); δ 6.96 (1H, s), 6.24 (1H, br), 5.30 (2H, s), 4.44 (2H, t), 4.38 (2H, t), 4.34 (2H, t), 3.66 (2H, q), 2.79 (2H, t), 2.49 (1H, m), 1.63˜1.88 (8H, m), 1.51 (2H, m), 0.96 (3H, t)

### Example 9-9

### Cyclohexanecarboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

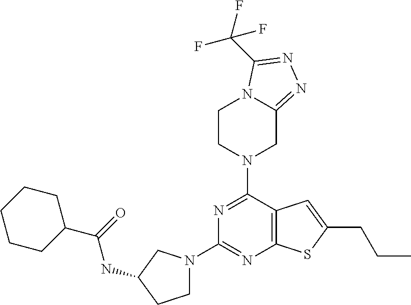

According to the same method to that described in Example 1-86, the title compound (80 mg, 37.4%) was obtained by using the compound (200 mg, 0.381 mmol) obtained from Example 1-14 and cyclohenxanecarboxylic acid (0.06 mL, 0.456 mmol).

¹H NMR (400 MHz, CDCl₃); δ 6.77 (1H, s), 6.32 (1H, d), 5.09 (2H, s), 4.54 (1H, m), 4.28 (2H, t), 4.08 (2H, t), 3.79 (1H, m), 3.57 (2H, m), 3.41 (1H, q), 2.75 (2H, t), 2.19 (1H, m), 2.07 (1H, m), 1.92 (1H, m), 1.61˜1.86 (7H, m), 1.38 (2H, m), 1.16˜1.35 (5H, m), 0.98 (3H, t)

### Example 9-10

### Cyclohexanecarboxylic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

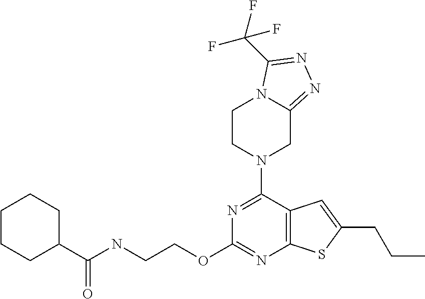

According to the same method to that described in Example 1-86, the title compound (43 mg, 19.5%) was obtained by using the compound (200 mg, 0.411 mmol) obtained from Example 3-58 and cyclohenxanecarboxylic acid (0.06 mL, 0.494 mmol).

¹H NMR (400 MHz, CDCl₃); δ 6.97 (1H, s), 6.22 (1H, br), 5.32 (2H, s), 4.45 (2H, t), 4.39 (2H, t), 4.36 (2H, t), 3.67 (2H, q), 2.83 (2H, t), 2.05 (1H, m), 1.65˜1.86 (7H, m), 1.14˜1.47 (5H, m), 0.98 (3H, t)

### Example 9-11

### 5-Chloro-thiophene-2-carboxylic Acid

### {(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-amide

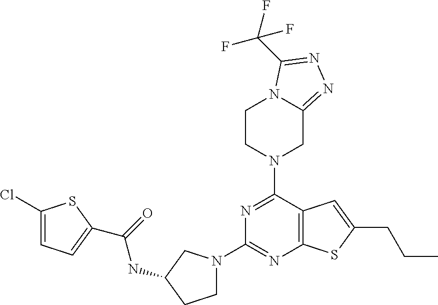

According to the same method to that described in Example 1-86, the title compound (150 mg, 66.0%) was obtained by using the compound (200 mg, 0.381 mmol) obtained from Example 1-14 and 5-chlorothiophene-2-carboxylic acid (0.074 g, 0.456 mmol).

¹H NMR (400 MHz, CDCl₃); δ 7.56 (1H, d), 7.49 (1H, m), 6.79 (1H, d), 6.70 (1H, s), 4.94 (2H, s), 4.72 (1H, br), 4.11 (2H, br), 3.98 (2H, br), 3.75 (1H, m), 3.60 (3H, br), 2.78 (2H, t), 2.23 (1H, m), 2.13 (1H, m), 1.70 (2H, m), 1.01 (3H, t)

### Example 9-12

### 5-Chloro-thiophene-2-carboxylic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

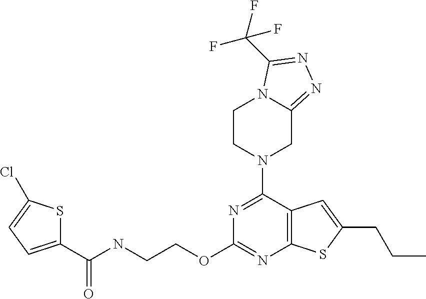

According to the same method to that described in Example 1-86, the title compound (64 mg, 27.2%) was obtained by using the compound (200 mg, 0.411 mmol) obtained from Example 3-58 and 5-chlorothiophene-2-carboxylic acid (0.08 g, 0.494 mmol).

¹H NMR (400 MHz, CDCl₃); δ 7.28 (1H, s), 6.94 (2H, br), 6.84 (1H, d), 5.29 (2H, s), 4.54 (2H, t), 4.37 (2H, t), 4.31 (2H, t), 3.80 (2H, q), 2.80 (2H, t), 1.70 (2H, m), 0.96 (3H, t)

### Example 9-13

### 3,4,5-Trifluoro-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-benzamide

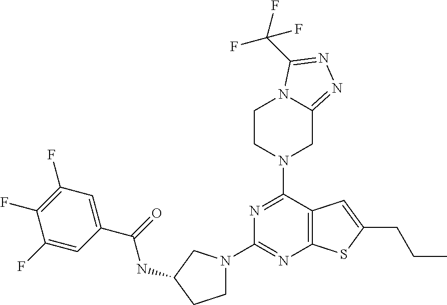

According to the same method to that described in Example 1-86, the title compound (125 mg, 53.9%) was obtained by using the compound (200 mg, 0.381 mmol) obtained from Example 1-14 and 3,4,5-trifluorobenzoic acid (0.08 g, 0.456 mmol).

¹H NMR (400 MHz, CDCl₃); δ 7.47 (2H, m), 6.75 (1H, s), 6.61 (1H, br), 5.09 (2H, s), 4.77 (1H, br), 4.21 (2H, t), 4.12 (2H, t), 3.85 (1H, m), 3.62 (3H, m), 2.78 (2H, t), 2.31 (1H, m), 2.12 (1H, m), 1.69 (2H, m), 0.99 (3H, t)

### Example 9-14

### 3,4,5-Trifluoro-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-benzamide

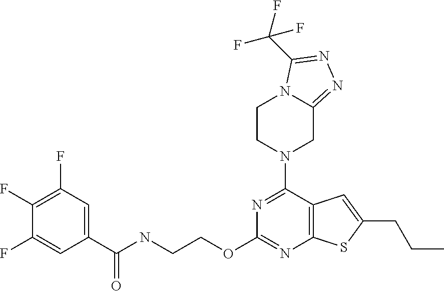

According to the same method to that described in Example 1-86, the title compound (66 mg, 27.4%) was obtained by using the compound (200 mg, 0.411 mmol) obtained from Example 3-58 and 3,4,5-trifluorobenzoic acid (0.087 g, 0.494 mmol).

¹H NMR (400 MHz, CDCl₃); δ 7.44 (3H, m), 6.95 (1H, s), 5.29 (2H, s), 4.57 (2H, t), 4.37 (2H, t), 4.34 (2H, t), 3.83 (2H, q), 2.80 (2H, t), 1.70 (2H, m), 0.99 (3H, t)

### Preparation Example 9-15-1

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid 4-nitro-phenyl ester

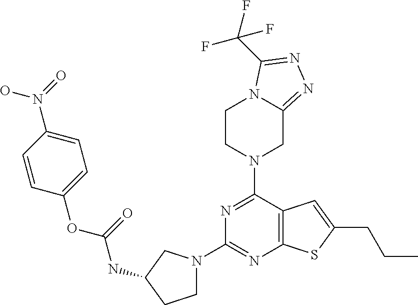

The compound (1 g, 1.900 mmol) obtained from Example 1-114 was dissolved in dichloromethane 100 mL and cooled to 0° C. Triethylamine (0.8 mL, 5.710 mmol) and 4-nitrophenylchloroformate (0.422 g, 2.094 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (600 mg, 51.3%) was obtained by column-chromatography using 3:1 mixture of dichloromethane and ethyl acetate.

¹H NMR (400 MHz, CDCl₃); δ 8.24 (2H, d), 7.32 (2H, d), 6.79 (1H, s), 5.31 (1H, br), 5.30 (2H, s), 4.46 (1H, br), 4.33 (2H, t), 4.21 (2H, t), 3.89 (1H, q), 3.72 (2H, t), 3.62 (1H, q), 2.77 (2H, t), 2.33 (1H, m), 2.05 (1H, m), 1.69 (2H, m), 0.98 (3H, t)

### Example 9-15

### 1-Cyclopentyl-3-{(S)-1-[6-propyl-4-(3-trifluoromethyl-5,6-dihydro-8H-[1,2,4]triazol o[4,3-a]pyrazin-7-yl)-thieno[2,3-d]pyrimidin-2-yl]-pyrrolidin-3-yl}-urea

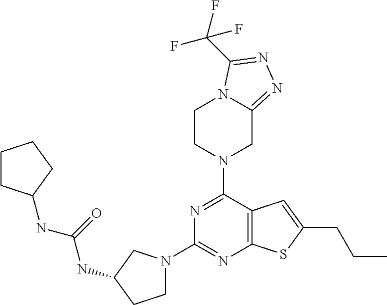

The compound (100 mg, 0.177 mmol) obtained from Preparation Example 9-15-1 was dissolved in dichloromethane 30 mL and cooled to 0° C. Triethylamine (0.075 mL, 0.5322 mmol) and cyclopentylamine (0.017 mL, 0.1774 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (70 mg, 70.0%) was obtained by column-chromatography using 20:1 mixture of dichloromethane and methanol.

¹H NMR (400 MHz, CDCl₃); δ 6.73 (1H, s), 5.58 (1H, br), 5.26 (1H, br), 5.06 (2H, br), 4.42 (1H, m), 4.28 (2H, t), 4.10 (2H, m), 4.00 (1H, m), 3.77 (1H, q), 3.53 (2H, m), 3.36 (1H, br), 2.73 (2H, t), 2.18 (1H, m), 1.87 (3H, m), 1.70 (2H, t), 1.53˜1.69 (4H, m), 1.35 (2H, m), 0.98 (3H, t)

### Example 9-16

### 1-(3,4-Difluoro-phenyl)-3-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-urea

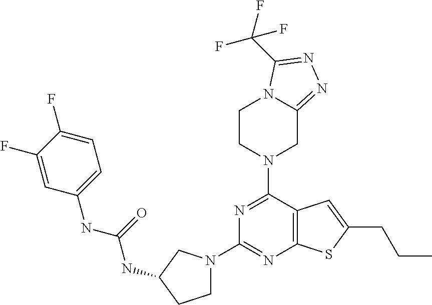

The compound (100 mg, 0.177 mmol) obtained from Preparation Example 9-15-1 was dissolved in dichloromethane 30 mL and cooled to 0° C. Triethylamine (0.075 mL, 0.5322 mmol) and 3,4-difluorophenylamine (0.018 mL, 0.1774 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (62 mg, 57.4%) was obtained by column-chromatography using 20:1 mixture of dichloromethane and methanol.

¹H NMR (400 MHz, CDCl₃); δ 7.98 (1H, d), 7.34 (1H, m), 6.86 (2H, m), 6.69 (1H, s), 6.43 (1H, br), 5.00 (2H, br), 4.45 (1H, br), 4.24 (2H, t), 4.06 (2H, m), 3.71 (1H, q), 3.57 (2H, m), 3.45 (1H, m), 2.70 (2H, t), 2.17 (1H, m), 1.95 (1H, m), 1.63 (2H, m), 0.97 (3H, t)

### Example 9-17

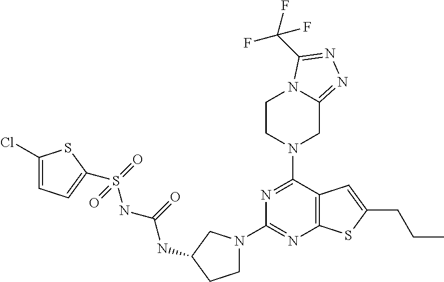

The compound (100 mg, 0.177 mmol) obtained from Preparation Example 9-15-1 was dissolved in dichloromethane 30 mL and cooled to 0° C. Triethylamine (0.075 mL, 0.5322 mmol) and 5-chlorothiophene-2-sulfonic acid amide (0.035 g, 0.1774 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (60 mg, 50.0%) was obtained by column-chromatography using 20:1 mixture of dichloromethane and methanol.

¹H NMR (400 MHz, CDCl₃); δ 7.45 (1H, d), 6.76 (3H, br), 5.07 (2H, s), 4.37 (1H, br), 4.29 (2H, br), 4.13 (2H, br), 3.72 (1H, m), 3.58 (2H, br), 3.46 (1H, br), 2.70 (2H, t), 2.17 (1H, m), 1.93 (1H, m), 1.63 (2H, m), 0.94 (3H, t)

### Example 9-18

### {(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester

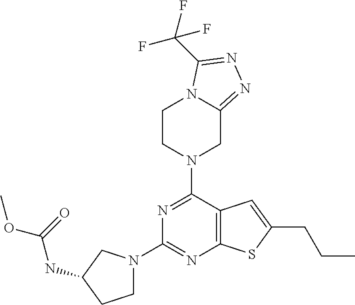

The compound (200 mg, 0.381 mmol) obtained from Example 1-14 was dissolved in dichloromethane 30 mL and cooled to 0° C. Triethylamine (0.16 mL, 1.142 mmol) and methylchloroformate (0.03 mL, 0.381 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (155 mg, 80.0%) was obtained by column-chromatography using 20:1 mixture of dichloromethane and methanol.

¹H NMR (400 MHz, CDCl₃); δ 6.78 (1H, s), 5.52 (1H, br), 5.30 (2H, s), 4.50 (1H, br), 4.31 (2H, br), 4.17 (2H, t), 3.80 (1H, m), 3.59 (5H, m), 3.52 (1H, br), 2.75 (2H, t), 2.20 (1H, m), 1.95 (1H, m), 1.67 (2H, m), 0.97 (3H, t)

### Example 9-19

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester

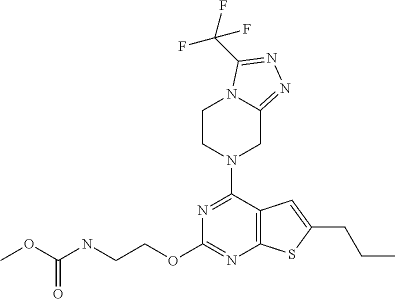

The compound (160 mg, 0.329 mmol) obtained from Example 3-58 was dissolved in dichloromethane 30 mL and cooled to 0° C. Triethylamine (0.17 mL, 1.234 mmol) and methylchloroformate (0.025 mL, 0.411 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (100 mg, 50.0%) was obtained by column-chromatography using 20:1 mixture of dichloromethane and methanol.

¹H NMR (400 MHz, CDCl₃); δ 6.96 (1H, s), 5.40 (1H, br), 5.29 (2H, s), 4.38 (2H, t), 4.33 (2H, t), 4.34 (2H, t), 3.62 (3H, s), 3.53 (2H, m), 2.81 (2H, t), 1.70 (2H, m), 0.99 (3H, t)

### Example 9-20

### {(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester

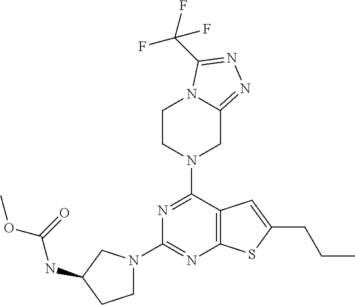

The compound (100 mg, 0.190 mmol) obtained from Example 1-13 was dissolved in dichloromethane 30 mL and cooled to 0° C. Triethylamine (0.08 mL, 0.571 mmol) and methylchloroformate (0.02 mL, 0.190 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (80 mg, 82.0%) was obtained by column-chromatography using 20:1 mixture of dichloromethane and methanol.

¹H NMR (400 MHz, CDCl₃); δ 6.78 (1H, s), 5.52 (1H, br), 5.30 (2H, s), 4.50 (1H, br), 4.31 (2H, br), 4.17 (2H, t), 3.80 (1H, m), 3.59 (5H, m), 3.52 (1H, br), 2.75 (2H, t), 2.20 (1H, m), 1.95 (1H, m), 1.67 (2H, m), 0.97 (3H, t)

### Example 9-21

### N-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide

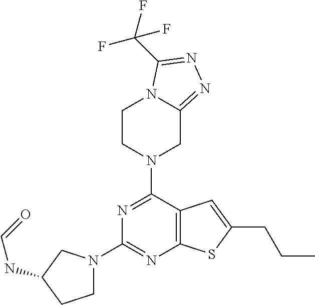

According to the same method to that described in Example 1-86, the title compound (37 mg, 40.4%) was obtained by using the compound (100 mg, 0.190 mmol) obtained from Example 1-14 and formic acid (0.01 mL, 0.228 mmol).

¹H NMR (400 MHz, CDCl₃); δ 8.17 (1H, s), 6.77 (1H, s), 6.21 (1H, br), 5.14 (2H, s), 4.63 (1H, br), 4.26 (2H, t), 4.17 (2H, t), 3.81 (1H, m), 3.62 (2H, t), 3.51 (1H, m), 2.75 (2H, t), 2.25 (1H, m), 1.98 (1H, m), 1.67 (2H, m), 0.98 (3H, t)

### Example 9-22

### N-{(R)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide

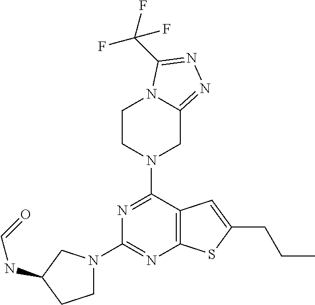

According to the same method to that described in Example 1-86, the title compound (41 mg, 45.0%) was obtained by using the compound (100 mg, 0.190 mmol) obtained from Example 1-13 and formic acid (0.01 mL, 0.228 mmol).

¹H NMR (400 MHz, CDCl₃); δ 8.17 (1H, s), 6.77 (1H, s), 6.21 (1H, br), 5.14 (2H, s), 4.63 (1H, br), 4.26 (2H, t), 4.17 (2H, t), 3.81 (1H, m), 3.62 (2H, t), 3.51 (1H, m), 2.75 (2H, t), 2.25 (1H, m), 1.98 (1H, m), 1.67 (2H, m), 0.98 (3H, t)

### Example 9-23

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide

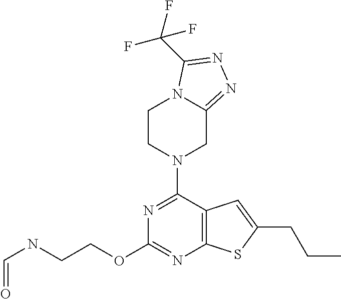

According to the same method to that described in Example 1-86, the title compound (55 mg, 60.0%) was obtained by using the compound (100 mg, 0.206 mmol) obtained from Example 3-58 and formic acid (0.01 mL, 0.247 mmol).

¹H NMR (400 MHz, CDCl₃); δ 8.22 (1H, s), 6.95 (1H, s), 6.14 (1H, br), 5.31 (2H, s), 4.47 (2H, t), 4.36 (2H, t), 4.33 (2H, t), 3.73 (2H, q), 2.82 (2H, t), 1.71 (2H, m), 1.00 (3H, t)

### Preparation Example 9-24-1

### Hydrochloric acid salt of N-Methyl-N—(S)-pyrrolidin-3-yl-acetamide

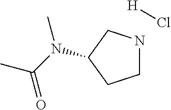

(S)-3-amino-pyrrolidine-1-carboxylic acid t-butyl ester (200 mg, 1.074 mmol) and acetic anhydride (0.1 mL, 1.074 mmol) were diluted with dichloromethane 30 mL. Triethylamine (0.75 mL, 5.369 mmol) was slowly added thereto and stirred for 16 hours. After the reaction mixture was distilled under reduced pressure, dissolved in dimethylformamide 20 mL and cooled to 0° C., sodium hydride (26.0 mg, 0.651 mmol) was added thereto and the reaction was carried out for 30 minutes. Iodomethane (0.041 mL, 0.651 mmol) was added to the reaction mixture and the reaction was carried out for 16 hours. The resulting solution was distilled under reduced pressure, diluted with ethyl acetate and washed with water and brine. After the organic layer was dried with anhydrous magnesium sulfate and distilled under reduced pressure, the column-chromatography using 3:1 mixture of hexane and ethyl acetate was carried out for purification. The purified compound was diluted with dichloromethane 30 mL. 4.0 M hydrochloric acid dioxane solution 1 mL was added thereto and stirred for 1 hour. After the solvent was removed by distillation under reduced pressure and solidified, the title compound (70 mg, 96%) was obtained by cleanse the solid with diethyl ether.

MS (M+1): 143.5

### Example 9-24

### N-Methyl-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

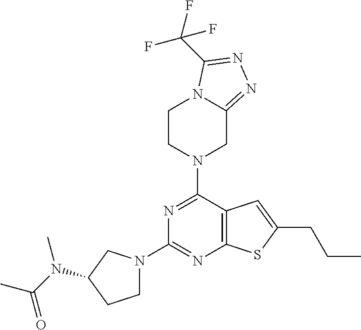

According to the same method to that in Example 1-1, the title compound (30 mg, 15%) was obtained by using the compound (70 mg, 0.392 mmol) obtained from Preparation Example 9-24-1, the compound (160 mg, 0.392 mmol) obtained from Preparation Example 1-1-3 and diisopropylethylamine (0.14 mL, 16.26 mmol).

¹H NMR (400 MHz, CDCl₃); δ 6.79 (1H, s), 5.36 (1H, br), 5.19 (2H, s), 4.52 (1H, br), 4.33 (2H, br), 4.19 (2H, t), 3.78 (2H, m), 3.41 (2H, m), 2.89 (3H, d), 2.72 (2H, t), 2.10˜2.21 (4H, m), 2.00 (1H, m), 1.68 (2H, m), 0.91 (3H, t)

### Preparation Example 9-25-1

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid 4-nitro-phenyl ester

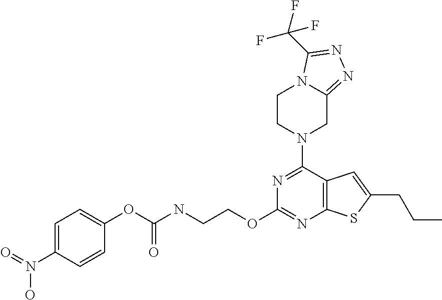

The compound (300 mg, 0.617 mmol) obtained from Example 3-58 was dissolved in dichloromethane 50 mL and cooled to 0° C. Triethylamine (0.26 mL, 1.851 mmol) and 4-nitorphenylchloroformate (0.137 g, 0.679 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (300 mg, 90.0%) was obtained by column-chromatography using 2:1 mixture of dichloromethane and ethyl acetate.

¹H NMR (400 MHz, CDCl₃); δ 8.23 (2H, d), 7.30 (2H, d), 6.96 (1H, s), 5.74 (1H, t), 5.32 (2H, s), 4.53 (1H, t), 4.37 (2H, t), 4.33 (2H, t), 3.71 (2H, q), 2.83 (2H, t), 1.72 (2H, m), 1.00 (3H, t)

### Example 9-25

### 1-Methyl-3-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-urea

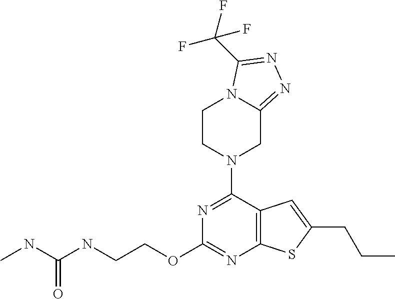

The compound (70 mg, 0.130 mmol) obtained from Preparation Example 9-25-1 was dissolved in dichloromethane 30 mL and cooled to 0° C. Triethylamine (0.06 mL, 0.390 mmol) and 2M methylamine tetrahydrofuran solution (0.06 mL, 0.130 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (60 mg, 95.0%) was obtained by column-chromatography using 20:1 mixture of dichloromethane and methanol.

¹H NMR (400 MHz, CDCl₃); δ 6.94 (1H, s), 5.31 (2H, s), 4.85 (1H, br), 4.44 (2H, t), 4.36 (2H, t), 4.32 (2H, t), 3.61 (2H, q), 2.82 (2H, t), 2.78 (3H, d), 1.72 (2H, m), 1.00 (3H, t)

### Example 9-26

### Pyrrolidine-1-carboxylic Acid

### {2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amide

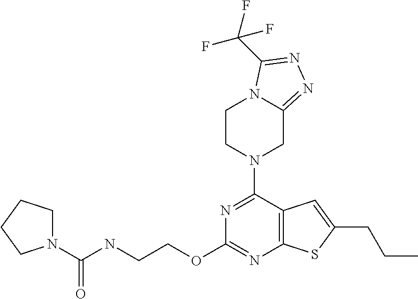

The compound (50 mg, 0.093 mmol) obtained from Preparation Example 9-25-1 was dissolved in dichloromethane 30 mL and cooled to 0° C. Triethylamine (0.04 mL, 0.279 mmol) and pyrrolidine (0.01 mL, 0.093 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (50 mg, 100.0%) was obtained by column-chromatography using 20:1 mixture of dichloromethane and methanol.

¹H NMR (400 MHz, CDCl₃); δ 6.95 (1H, s), 5.30 (2H, s), 4.95 (1H, br), 4.46 (2H, t), 4.34 (2H, t), 4.32 (2H, t), 3.67 (2H, q), 3.31 (4H, br), 2.81 (2H, t), 1.87 (4H, br), 1.70 (2H, m), 0.99 (3H, t)

### Example 9-27

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-urea

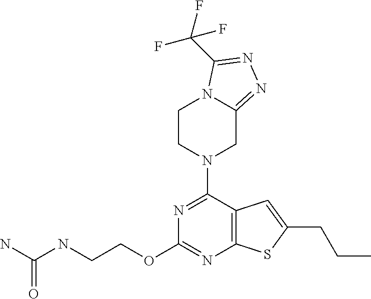

The compound (150 mg, 0.279 mmol) obtained from Preparation Example 9-25-1 was dissolved in dichloromethane 30 mL and cooled to 0° C. Triethylamine (0.04 mL, 0.836 mmol) and 7N ammonia methanol solution (0.1 mL, 0.4178 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (46 mg, 35.0%) was obtained by column-chromatography using 20:1 mixture of dichloromethane and methanol.

¹H NMR (400 MHz, CDCl₃); δ 6.90 (1H, s), 5.76 (1H, br), 5.26 (2H, s), 4.41 (2H, t), 4.36 (2H, t), 4.30 (2H, t), 3.57 (2H, q), 2.78 (2H, t), 1.68 (2H, m), 0.98 (3H, t)

### Example 9-28

### N-{4-Hydroxy-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-formamide

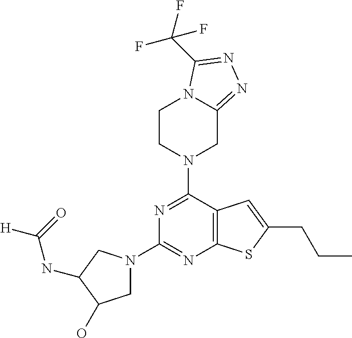

According to the same method to that described in Example 1-86, the title compound (270 mg, 30.0%) was obtained by using the compound (1 g, 1.847 mmol) obtained from Example 1-114 and formic acid (0.09 mL, 2.216 mmol).

¹H NMR (400 MHz, CDCl₃); δ 8.22 (1H, s), 6.95 (1H, s), 6.14 (1H, br), 5.31 (2H, s), 4.47 (2H, t), 4.36 (2H, t), 4.33 (2H, t), 3.73 (2H, q), 2.82 (2H, t), 1.71 (2H, m), 1.00 (3H, t)

¹H NMR (400 MHz, CDCl₃); δ 8.14 (1H, s), 6.83 (1H, br), 6.74 (1H, s), 5.09 (2H, s), 4.33 (2H, m), 4.24 (2H, t), 4.11 (2H, t), 3.99 (1H, m), 3.89 (1H, m), 3.51 (2H, m), 2.73 (2H, t), 1.66 (2H, m), 0.97 (3H, t)

### Example 9-29

### N-{4-Chloro-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

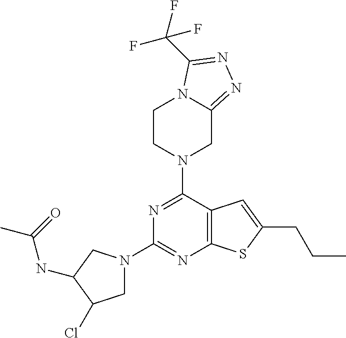

The compound (60 mg, 0.131 mmol) obtained from Example 7-17 was dissolved in toluene 20 mL. Phosphorous oxychloride (0.05 mL, 0.526 mmol) was added thereto and the reaction was carried out for 3 hours at 100° C. The reaction mixture was cooled to room temperature, distilled under reduced pressure, diluted with ethyl acetate and washed with water and brine. After the organic layer was dried with anhydrous magnesium sulfate and distilled under reduced pressure, the title compound (6.4 mg, 10%) was obtained by column-chromatography using 20:1 mixture of dichloromethane and methanol.

¹H NMR (400 MHz, CDCl₃); δ 6.77 (1H, s), 6.15 (1H, br), 5.10 (2H, s), 4.54 (1H, br), 4.43 (1H, br), 4.22 (2H, br), 4.01˜4.15 (5H, m), 3.88 (1H, m), 3.60 (1H, d), 2.78 (2H, t), 2.02 (3H, s), 1.70 (2H, m), 1.00 (3H, t)

### Example 9-30

### 7-[2-(2-Methyl-3a,4,6,6a-tetrahydro-pyrrolo[3,4-d]oxazol-5-yl)-6-propyl-thieno[2,3-d]pyrimidin-4-yl]-3-trifluoromethyl-5,6,7,8-tetrahydro-[1,2,4]triazolo[4,3-a]pyrazine

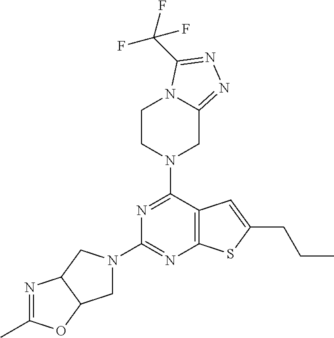

The compound (130 mg, 0.285 mmol) obtained from Example 7-17 was dissolved in dichloromethane 50 mL and cooled to 0° C. Diisopropylethylamine (0.075 mL, 0.427 mmol) and methanesulfonylchloride (0.025 mL, 0.313 mmol) were slowly added thereto. After the reaction was carried out for 1 hours at room temperature, additional reaction was carried out for 3 hours with addition of 1,8-diazabicyclo[5,4,0]undec-7-ene and then, the reaction mixture was washed with water and brine. After the organic layer was dried with anhydrous magnesium sulfate and distilled under reduced pressure, the title compound (52 mg, 37.1%) was obtained by column-chromatography using 20:1 mixture of dichloromethane and methanol.

¹H NMR (400 MHz, CDCl₃); δ 6.80 (1H, s), 5.19 (2H, s), 5.09 (1H, m), 4.71 (1H, t), 4.34 (2H, t), 4.19 (2H, t), 4.08 (1H, d), 3.92 (1H, d), 3.50 (2H, m), 2.76 (2H, t), 1.96 (3H, s), 1.65 (2H, m), 0.97 (3H, t)

### Example 9-31

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-formamide

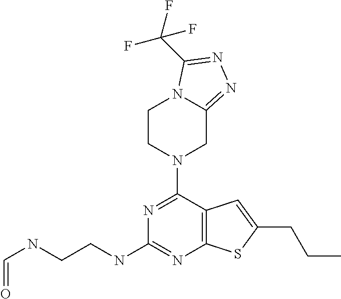

According to the same method to that described in Example 1-86, the title compound (19 mg, 23.2%) was obtained by using the compound (90 mg, 0.180 mmol) obtained from Example 7-38 and formic acid (0.008 mL, 0.216 mmol).

¹H NMR (500 MHz, CDCl₃); δ 8.15 (1H, s), 6.78 (1H, d), 5.43 (1H, br), 5.11 (2H, d), 4.30 (2H, t), 4.20 (2H, t), 3.55 (2H, t), 3.46 (2H, t), 2.73 (2H, q), 1.67 (2H, m), 0.96 (3H, t)

### Example 9-32

### {2-[6-Propyl-4-(3-trifluoromethyl-5,6-dihydro-8H-[1,2,4]triazolo[4,3-a]pyrazin-7-yl)-thieno[2,3-d]pyrimidin-2-ylamino]-ethyl}-carbamic acid methyl ester

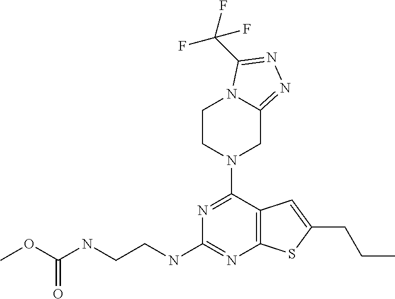

The compound (90 mg, 0.180 mmol) obtained from Example 7-38 was dissolved in dichloromethane 20 mL and cooled to 0° C. Triethylamine (0.075 mL, 0.180 mmol) and methylchloroformate (0.014 mL, 0.180 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (66 mg, 76.0%) was obtained by column-chromatography using 20:1 mixture of dichloromethane and methanol.

¹H NMR (500 MHz, CDCl₃); δ 6.98 (1H, s), 5.67 (2H, br), 5.25 (2H, s), 4.33 (2H, t), 4.14 (2H, t), 3.64 (3H, s), 3.53 (2H, m), 3.39 (2H, q), 2.74 (2H, t), 1.66 (2H, m), 0.94 (3H, t)

### Example 9-33

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-imidazolidin-2-one

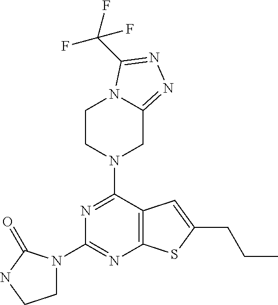

The compound (100 mg, 0.206 mmol) obtained from Example 7-38 was dissolved in dichloromethane 20 mL and cooled to 0° C. Triethylamine (0.086 mL, 0.618 mmol) and 4-nitrophenylchloroformate (0.046 g, 0.269 mmol) were added thereto and stirred for 3 hours at room temperature. The reaction solution was cooled to 0° C. Triethylamine (0.086 mL, 0.618 mmol) and 7 N ammonia methanol solution (0.05 mL, 0.309 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (45 mg, 46.4%) was obtained by column-chromatography using 20:1 mixture of dichloromethane and methanol.

¹H NMR (500 MHz, DMSO); δ 7.39 (1H, s), 5.67 (2H, br), 5.25 (2H, s), 4.39 (2H, t), 4.29 (2H, t), 3.98 (2H, t), 3.38 (2H, m), 2.83 (2H, t), 1.67 (2H, m), 0.94 (3H, t)

### Example 9-34

### 7-[6-Propyl-2-(3a,4,6,6a-tetrahydro-pyrrolo[3,4-d]oxazol-5-yl)-thieno[2,3-d]pyrimidin-4-yl]-3-trifluoromethyl-5,6,7,8-tetrahydro-[1,2,4]triazolo[4,3-a]pyrazine

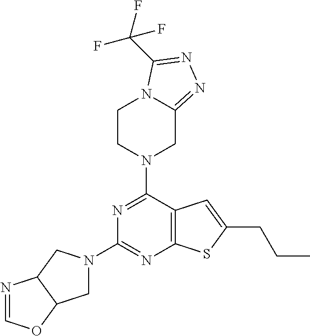

According to the same method to that described in Example 9-30, the title compound (9 mg, 9.0%) was obtained by using the compound (120 mg, 0.242 mmol) obtained from Example 9-28.

¹H NMR (400 MHz, CDCl₃); δ 8.26 (1H, s), 6.79 (1H, br), 6.72 (1H, s), 5.19 (1H, br), 5.01 (2H, s), 4.59 (1H, br), 3.92˜4.16 (7H, m), 3.71 (1H, br), 2.78 (2H, t), 1.70 (2H, m), 0.97 (3H, t)

### Example 9-35

### 7-(2-Methoxy-6-propyl-thieno[2,3-d]pyrimidin-4-yl)-3-trifluoromethyl-5,6,7,8-tetrahydro-[1,2,4]triazolo[4,3-a]pyrazine

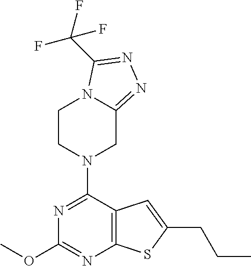

According to the same method to that described in Example 1-45, the title compound (200 mg, 41%) was obtained by using the compound (500 mg, 1.241 mmol) obtained from Preparation Example 1-1-3 and methanol (0.08 mL, 1.862 mmol).

¹H NMR (500 MHz, CDCl₃); δ 6.93 (1H, s), 5.29 (2H, s), 4.36 (2H, t), 4.29 (2H, t), 4.00 (3H, s), 2.81 (2H, t), 1.72 (2H, m), 0.99 (3H, t)

### Example 9-36

### 7-(2-Ethoxy-6-propyl-thieno[2,3-d]pyrimidin-4-yl)-3-trifluoromethyl-5,6,7,8-tetrahydro-[1,2,4]triazolo[4,3-a]pyrazine

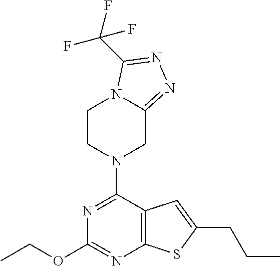

According to the same method to that described in Example 1-45, the title compound (61 mg, 30%) was obtained by using the compound (200 mg, 0.497 mmol) obtained from Preparation Example 1-1-3 and ethanol (0.044 mL, 0.745 mmol).

¹H NMR (500 MHz, CDCl₃); δ 6.93 (1H, s), 5.29 (2H, s), 4.40 (2H, t), 4.36 (2H, t), 4.29 (2H, t), 2.81 (2H, t), 1.71 (2H, m), 1.30 (3H, t), 1.00 (3H, t)

### Example 9-37

### 7-(2-Azido-6-propyl-thieno[2,3-d]pyrimidin-4-yl)-3-trifluoromethyl-5,6,7,8-tetrahydro-[1,2,4]triazolo[4,3-a]pyrazine

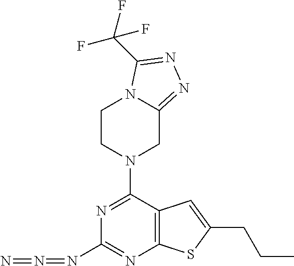

The compound (2.0 g, 4.965 mmol) obtained from Preparation Example 1-1-3 was dissolved in N-methyl-2-pyrrolidinone 30 mL and water 10 mL. Sodium azide (1.614 g, 24.824 mmol) was added thereto and the reaction was carried out for 24 hours at 150° C. The reaction solution was distilled under reduced pressure, diluted with ethyl acetate and washed with water and brine. After the organic layer was dried with anhydrous magnesium sulfate and distilled under reduced pressure, the title compound (1.2 g, 59%) was obtained by column-chromatography using 2:1 mixture of hexane and ethyl acetate.

¹H NMR (500 MHz, CDCl₃); δ 6.99 (1H, s), 5.30 (2H, s), 4.38 (2H, t), 4.33 (2H, t), 2.85 (2H, t), 1.72 (2H, m), 1.00 (3H, t)

### Example 9-38

### 6-Propyl-4-(3-trifluoromethyl-5,6-dihydro-8H-[1,2,4]triazolo[4,3-a]pyrazin-7-yl)-thieno[2,3-d]pyrimidin-2-ylamine

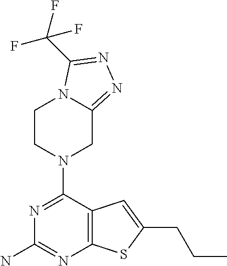

The compound (310 mg, 0.757 mmol) obtained from Example 9-37 was dissolved in tetrahydrofuran 30 mL and cooled to 0° C. Trimethylphosphine 1M tetrahydrofuran solution (1.04 mL, 0.91 mmol) was slowly added thereto and the reaction was carried out for 16 hours. The reaction solution was distilled under reduced pressure, diluted with 20:1 mixture of dichloromethane and methanol, and washed with water and brine. The organic layer was dried with anhydrous magnesium sulfate and distilled under reduced pressure to give the title compound (270 mg, 93%).

¹H NMR (500 MHz, DMSO); δ 7.14 (1H, s), 6.31 (2H, s), 5.06 (2H, s), 4.31 (2H, t), 4.14 (2H, t), 2.70 (2H, t), 1.61 (2H, m), 0.91 (3H, t)

### Example 9-39

### N-[6-Propyl-4-(3-trifluoromethyl-5,6-dihydro-8H-[1,2,4]triazolo[4,3-a]pyrazin-7-yl)-thieno[2,3-d]pyrimidin-2-yl]-acetamide

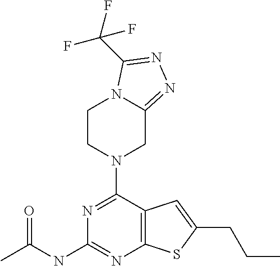

The compound (300 mg, 0.783 mmol) obtained from Example 9-38 was dissolved in pyridine 30 mL and cooled to 0° C. Acetyl chloride (0.11 mL, 1.565 mmol) was slowly added thereto and the reaction was carried out for 16 hours. The reaction solution was distilled under reduced pressure, diluted with dichloromethane and washed with water and brine. After the organic layer was dried with anhydrous magnesium sulfate and distilled under reduced pressure, the title compound (47 mg, 14.2%) was obtained by column-chromatography using 2:1 mixture of dichloromethane and ethyl acetate.

¹H NMR (500 MHz, DMSO); δ 6.96 (1H, s), 5.29 (2H, s), 4.39 (2H, t), 4.30 (2H, t), 2.84 (2H, t), 2.49 (3H, s), 1.69 (2H, m), 0.98 (3H, t)

### Preparation Example 9-40-1

### (2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-carbamic acid tert-butyl ester

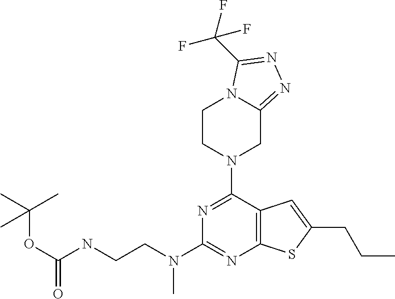

The compound (2 g, 4.965 mmol) obtained from Preparation Example 1-1-3 and

N*1*-methyl-ethane-1,2-diamine (0.73 g, 9.93 mmol) were dissolved in n-butanol 50 mL. The reaction was carried out for 16 hours at 150° C. After the reaction solution was distilled under reduced pressure, the remaining residue was diluted with dichloromethane 40 mL. Triethylamine (2.1 mL, 14.89 mmol) and di t-butyl dicarbonate (3.25 g, 14.89 mmol) were added thereto and then, the reaction was carried out for 3 hours. After the reaction solution was distilled under reduced pressure, the title compound (1.54 g, 57.5%) was obtained by column-chromatography using 3:1 mixture of hexane and ethyl acetate.

¹H NMR (500 MHz, CDCl₃); δ 6.77 (1H, s), 5.17 (2H, s), 5.04 (1H, br), 4.33 (2H, t), 4.22 (2H, t), 3.70 (2H, t), 3.31 (2H, q), 3.17 (3H, s), 2.75 (2H, t), 1.69 (2H, m), 1.38 (9H, s), 0.97 (3H, t)

### Preparation Example 9-40-2

### Hydrochloric Acid Salt of

### N*1*-Methyl-N*1*-[6-propyl-4-(3-trifluoromethyl-5,6-dihydro-8H-[1,2,4]triazolo[4,3-a]pyrazin-7-yl)-thieno[2,3-d]pyrimidin-2-yl]-ethane-1,2-diamine

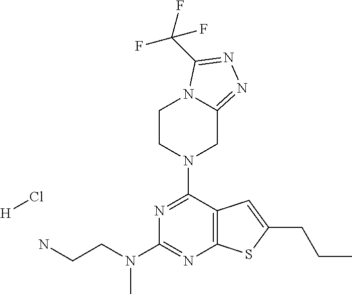

According to the same method to that described in Example 1-4, the title compound (1.21 g, 90%) was obtained by using the compound (1.54 g, 2.849 mmol) obtained from Preparation Example 9-40-1.

MS (M+1): 441.2

### Example 9-40

### N-(2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-acetamide

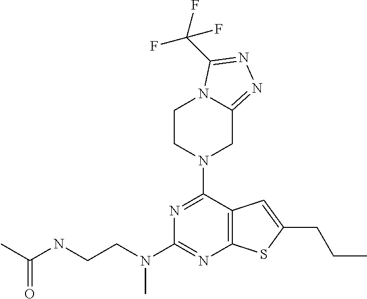

According to the same method to that described in Example 1-105, the title compound (80 mg, 40%) was obtained by using the compound (200 mg, 0.419 mmol) obtained from Preparation Example 9-40-2 and acetic anhydride (0.053 mL, 0.567 mmol).

¹H NMR (500 MHz, CDCl₃); δ 7.01 (1H, s), 5.16 (2H, s), 4.32 (2H, t), 4.23 (2H, t), 3.72 (2H, t), 3.40 (2H, q), 3.18 (3H, s), 2.77 (2H, t), 1.87 (3H, s), 1.68 (2H, m), 0.98 (3H, t)

### Example 9-41

### N-(2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-formamide

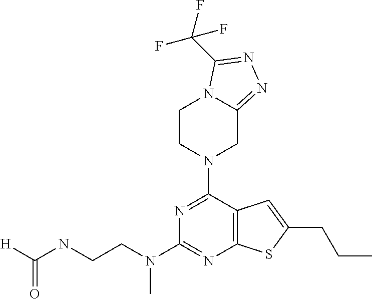

According to the same method to that described in Example 1-86, the title compound (57 mg, 30%) was obtained by using the compound (200 mg, 0.419 mmol) obtained from Preparation Example 9-40-2 and formic acid (0.036 mL, 0.946 mmol).

¹H NMR (500 MHz, CDCl₃); δ 8.14 (1H, s), 6.77 (1H, s), 5.15 (2H, s), 4.30 (2H, t), 4.23 (2H, t), 3.75 (2H, t), 3.48 (2H, q), 3.18 (3H, s), 2.76 (2H, t), 1.68 (2H, m), 0.99 (3H, t)

### Example 9-42

### (2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethyl)-carbamic acid methyl ester

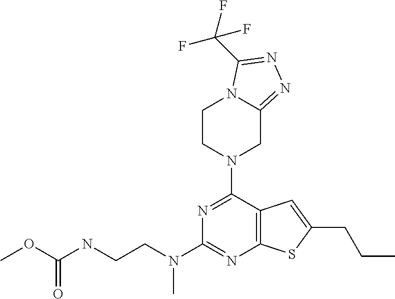

The compound (200 mg, 0.419 mmol) obtained from Preparation Example 9-40-2 was dissolved in dichloromethane 30 mL and cooled to 0° C. Triethylamine (0.198 mL, 1.258 mmol) and methylchloroformate (0.044 mL, 0.567 mmol) were added thereto and stirred for 3 hours at room temperature. The reaction solution was distilled under reduced pressure, diluted with dichloromethane and washed with water and brine. After the organic layer was dried with anhydrous magnesium sulfate and distilled under reduced pressure, the title compound (130 mg, 62.2%) was obtained by column-chromatography using 20:1 mixture of dichloromethane and methanol.

¹H NMR (500 MHz, CDCl₃); δ 6.78 (1H, s), 5.17 (2H, s), 4.31 (2H, t), 4.23 (2H, t), 3.73 (2H, t), 3.63 (3H, s), 3.38 (2H, q), 3.18 (3H, s), 2.76 (2H, t), 1.68 (2H, m), 0.98 (3H, t)

### Example 9-43

### 7-(6-Propyl-2-[1,2,4]triazol-1-yl-thieno[2,3-d]pyrimidin-4-yl)-3-trifluoromethyl-5,6,7,8-tetrahydro-[1,2,4]triazolo[4,3-a]pyrazine

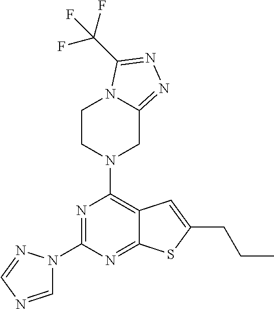

The compound (0.30 g, 0.74 mmol) obtained from Preparation Example 1-1-3 and 1,2,4-triazole (0.07 g, 1.04 mmol) were dissolved in toluene 5 mL. Sodium carbonate (0.11 g, 1.04 mmol), bis(dibenzylideneacetone) dipalladium (40 mg, 0.044 mmol) and 4,5-bis(diphenylphosphino)-9,9-dimethylxanthine (40 mg, 0.066 mmol) were added thereto and stirred for 4 hours with reflux. After the reaction was completed, methylene dichloride 20 mL was added thereto and filtered through Celite to remove the solid. The filtered solid was washed with water 10 mL. The concentration procedure was followed by the column-chromatography (developing solution: 5% methanol/methylene dichloride) to give the title compound (0.22 g, 0.50 mmol, yield: 68%) of white solid form.

¹H NMR (400 MHz, CDCl₃); δ 9.16 (1H, s), 8.16 (1H, s), 7.10 (1H, s), 5.42 (2H, s), 4.47 (4H, br), 2.92 (2H, t), 2.79 (2H, t), 1.82 (2H, m), 1.05 (3H, t)

### Preparation Example 9-44-1

### 2-[1,2,4]-Triazol-1-yl-ethanol

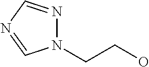

Sodium 1,2,4-triazole salt (1.50 g, 16.47 mmol) and 2-chloroethanol (2.65 g, 32.95 mmol) were dissolved in methanol 30 mL and stirred for 8 hours with refulx. After the reaction was completed, the solvent was removed. The remaining residue was diluted with ethyl acetate and washed with water. After the reaction mixture was dried with magnesium sulfate, the title compound (0.45 g, 3.98 mmol, yield: 24%) of yellow liquid form was obtained by the concentration procedure.

¹H NMR (400 MHz, CDCl₃); δ 8.15 (1H, s), 7.95 (1H, s), 4.34 (2H, t), 4.11 (2H, m), 2.28 (1H, br)

### Example 9-44

### 7-[6-Propyl-2-(2-[1,2,4]triazol-1-yl-ethoxy)-thieno[2,3-d]pyrimidin-4-yl]-3-trifluoromethyl-5,6,7,8-tetrahydro-[1,2,4]triazolo[4,3-a]pyrazine

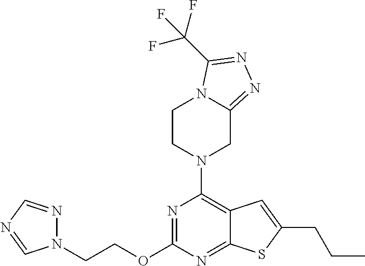

According to the same method to that described in Example 9-43, the reaction using the compound (0.30 g, 0.74 mmol) obtained from Preparation Example 1-1-3 and 2-[1,2,4]triazol-1-yl-ethanol (117 mg, 1.04 mmol) obtained from Preparation Example 9-44-1 instead of 1,2,4-triazole was carried out and then, the title compound (160 mg, 0.33 mmol, yield: 45%) of white solid form was obtained by the same treatment procedure to that described in Example 9-43.

¹H NMR (400 MHz, CDCl₃); δ 8.20 (1H, s), 7.95 (1H, s), 6.94 (1H, s), 5.30 (2H, s), 4.77 (2H, t), 4.61 (2H, t), 4.36 (2H, t) 4.32 (2H, t), 2.83 (2H, t), 1.78 (2H, m), 1.01 (3H, t)

### Example 9-45

### [2-(1H-Imidazol-4-yl)-ethyl]-[6-propyl-4-(3-trifluoromethyl-5,6-dihydro-8H-[1,2,4]triazolo[4,3-a]pyrazin-7-yl)-thieno[2,3-d]pyrimidin-2-yl]-amine

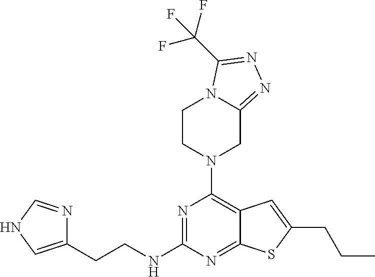

According to the same method to that described in Example 9-43, the reaction using the compound (0.30 g, 0.74 mmol) obtained from Preparation Example 1-1-3 and 2-(1H-imidazol-4-yl)-ethylamine (115 mg, 1.04 mmol) obtained from Preparation Example 9-44-1 instead of 1,2,4-triazole was carried out and then the title compound (107 mg, 0.22 mmol, yield: 30%) of white solid form was obtained by the same treatment procedure to that described in Example 9-43.

¹H NMR (400 MHz, CDCl₃); δ 7.61 (1H, s), 6.85 (1H, s), 6.79 (1H, s), 5.31 (1H, m), 5.16 (2H, s), 4.31 (2H, t), 4.21 (2H, t), 3.66 (2H, m), 2.90 (2H, t), 2.77 (2H, t), 1.75 (2H, m), 1.01 (3H, t)

### Example 9-46

### 2-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-imidazol-4-yl}-ethylamine

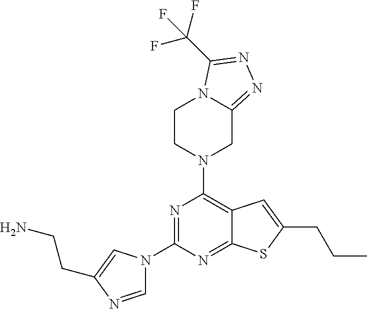

According to the same method to that described in Example 9-43, the reaction using the compound (0.30 g, 0.74 mmol) obtained from Preparation Example 1-1-3 and 2-(1H-imidazol-4-yl)-ethylamine (115 mg, 1.04 mmol) obtained from Preparation Example 9-44-1 instead of 1,2,4-triazole was carried out and then the title compound (125 mg, 0.25 mmol, yield: 35%) of white solid form was obtained by the same treatment procedure to that described in Example 9-43.

¹H NMR (400 MHz, CDCl₃); δ 8.40 (1H, s), 7.64 (1H, s), 6.97 (1H, s), 5.30 (2H, s), 4.41 (2H, t), 4.39 (2H, t), 3.32 (2H, t), 2.95 (2H, m), 2.86 (2H, t), 2.77 (2H, t), 1.75 (2H, m), 1.01 (3H, t)

### Example 9-47

### 7-[2-(3-Nitro-pyrrole-1-yl)-6-propyl-thieno[2,3-d]pyrimidin-4-yl]-3-trifluoromethyl-5,6,7,8-tetrahydro-[1,2,4]triazolo[4,3-a]pyrazine

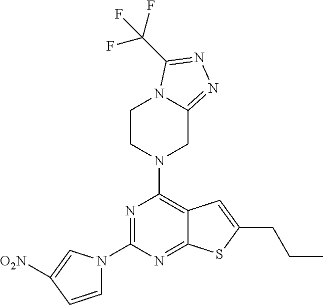

According to the same method to that described in Example 9-43, the reaction using the compound (0.30 g, 0.74 mmol) obtained from Preparation Example 1-1-3 and 3-nitropyrrole (117 mg, 1.04 mmol) obtained from Preparation Example 9-44-1 instead of 1,2,4-triazole was carried out and then the title compound (150 mg, 0.31 mmol, yield: 42%) of white solid form was obtained by the same treatment procedure to that described in Example 9-43.

¹H NMR (400 MHz, CDCl₃); δ 8.56 (1H, s), 7.73 (1H, s), 7.07 (1H, s), 6.87 (1H, s), 5.41 (2H, s), 4.47 (4H, br), 2.91 (2H, t), 1.79 (2H, m), 1.04 (3H, t)

### Example 9-48

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-ylamine

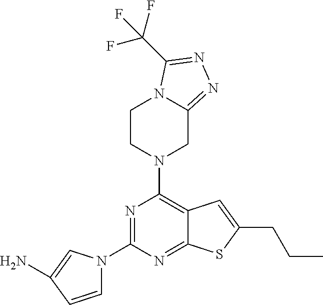

The compound (50 mg, 0.10 mmol) obtained from Example 9-47 was dissolved in ethyl acetate 10 mL. Tin chloride hydrate (0.10 g, 0.40 mmol) and a small amount of concentrated hydrochloric acid were added thereto and stirred at room temperature. After the reaction was completed, the resulting mixture was diluted ethyl acetate, washed with water and sodium hydrogen carbonate, dried and concentrated. The title compound (10 mg, 0.02 mmol, yield: 22%) was obtained by Prep. HPLC and freeze drying.

¹H NMR (400 MHz, DMSO-d₆, TFA salt); δ 7.80 (2H, m), 7.54 (1H, s), 6.33 (1H, m), 5.34 (2H, s), 4.40 (4H, br), 2.90 (2H, t), 1.73 (2H, m), 0.98 (3H, t)

### Example 9-49

### N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-yl}-acetamide

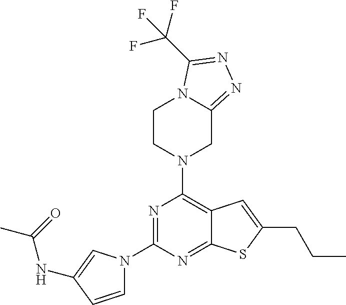

The compound (0.25 g, 0.56 mmol) obtained from Example 9-48 was dissolved in dichloromethane 5 mL. Triethylamine (0.16 g, 1.66 mmol) was added thereto and acetic anhydride (57 mg, 0.56 mmol) was slowly added dropwise thereto at 0° C. After the reaction was completed, the resulting mixture was diluted with dichloromethane, washed with sodium hydrogen carbonate aqueous solution and brine and dried with magnesium sulfate. After the solvent was removed, the title compound (20 mg, 0.04 mmol, yield: 7%) of white solid form was obtained by the column-chromatography (developing solution; 2:1 mixture of ethyl acetate and normal hexane).

¹H NMR (400 MHz, DMSO-d₆); δ 9.92 (1H, s), 7.98 (1H, d), 7.66 (1H, m), 7.48 (1H, s), 5.37 (2H, s), 4.40 (2H, m), 4.38 (2H, m), 2.89 (2H, t), 1.73 (2H, m), 0.97 (3H, t)

### Preparation Example 9-50-1

### 2-(Pyrazin-2-ylamino)-ethanol

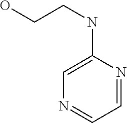

2-Chloropyrazine (1.0 g, 8.73 mmol) was added to monoethanol amine (1.08 g, 17.46 mmol) and stirred with reflux under the absence of solvent. After the reaction was completed, the concentratin procedure was followed by the column-chromatography (developing solution; 10% methanol/dichloromethane) to give the title compound (1.21 g, 8.70 mmol, yield: 99%) of yellow liquid form.

¹H NMR (400 MHz, CDCl₃); δ 7.95 (2H, s), 7.82 (1H, s), 4.99 (1H, br), 3.85 (2H, t), 3.58 (2H, m), 3.16 (1H, br)

### Example 9-50

### 2-{[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrazin-2-yl-amino}-ethanol

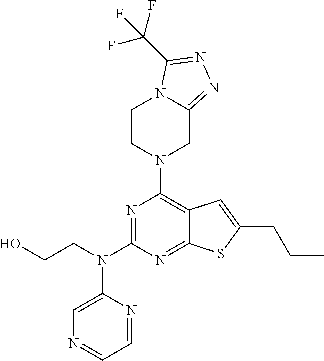

According to the same method to that described in Example 9-43, the reaction using the compound (0.30 g, 0.74 mmol) obtained from Preparation Example 1-1-3 and 2-(pyrazin-2-ylamino)-ethanol (145 mg, 1.04 mmol) obtained from Preparation Example 9-50-1 instead of 1,2,4-triazole was carried out and then the title compound (150 mg, 0.30 mmol, yield: 41%) of white solid form was obtained by the same treatment procedure to that described in Example 9-43.

¹H NMR (400 MHz, CDCl₃); δ 9.05 (1H, s), 8.30 (1H, m), 8.24 (1H, d), 6.93 (1H, s), 5.21 (2H, s), 4.92 (1H, m), 4.46 (2H, m), 4.29 (2H, m), 4.19 (2H, m), 3.92 (2H, m), 2.84 (2H, t), 1.73 (2H, m), 1.04 (3H, t)

### Preparation Example 9-51-1

### 3-(2-Hydroxy-ethyl)-imidazolidine-2,4-dione

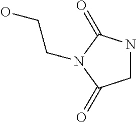

Hydantoin (3.0 g, 29.98 mmol) was dissolved in dimethylformamide 30 mL. 2-Chloroethanol (2.65 g, 32.97 mmol) and potassium carbonate (4.56 g, 32.97 mmol) were added thereto and stirred with heating to 100° C. After the reaction was completed, the solvent was removed. The remaining residue was diluted with ethyl acetate, washed with 1N hydrochloric acid aqueous solution and water and dried with magnesium sulfate. The title compound (0.5 g, 3.47 mmol, yield: 12%) of yellow liquid form was obtained by the concentration procedure.

MS (M+1): 145.2

### Example 9-51

### 3-(2-Hydroxy-ethyl)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-imidazolidine-2,4-dione

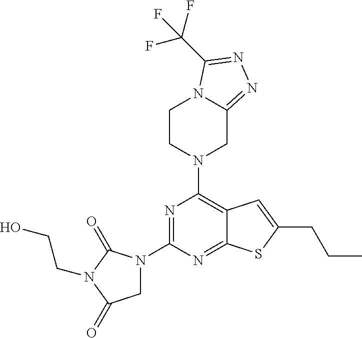

According to the same method to that described in Example 9-43, the reaction using the compound (0.30 g, 0.74 mmol) obtained from Preparation Example 1-1-3 and 3-(2-hydroxy-ethyl)-imidazoline-2,4-dione (150 mg, 1.04 mmol) obtained from Preparation Example 9-51-1 instead of 1,2,4-triazole was carried out and then the title compound (230 mg, 0.45 mmol, yield: 61%) of white solid form was obtained by the same treatment procedure to that described in Example 9-43.

¹H NMR (400 MHz, DMSO-d₆); δ 7.48 (1H, s), 5.32 (2H, s), 4.83 (1H, t), 4.53 (2H, s), 4.41 (2H, m), 4.36 (2H, m), 3.56 (4H, m), 2.87 (2H, t), 1.72 (2H, m), 0.98 (3H, t)

### Example 9-52

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-ylamine

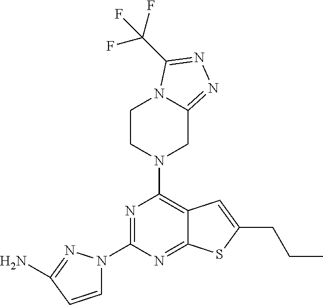

According to the same method to that described in Example 9-43, the reaction using the compound (0.30 g, 0.74 mmol) obtained from Preparation Example 1-1-3 and 3-aminopyrazole (86 mg, 1.04 mmol) instead of 1,2,4-triazole was carried out and then the title compound (60 mg, 0.13 mmol, yield: 18%) of white solid form was obtained by the same treatment procedure to that described in Example 9-43.

¹H NMR (400 MHz, CDCl₃); δ 8.27 (1H, d), 6.97 (1H, s), 5.97 (1H, d), 5.32 (2H, s), 4.40 (2H, m), 4.37 (2H, m), 4.03 (2H, br), 2.86 (2H, t), 1.78 (2H, m), 1.04 (3H, t)

### Example 9-53

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(1H-pyrazol-3-yl)-amine

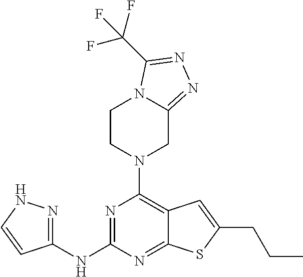

According to the same method to that described in Example 9-43, the reaction using the compound (0.30 g, 0.74 mmol) obtained from Preparation Example 1-1-3 and 3-aminopyrazole (86 mg, 1.04 mmol) instead of 1,2,4-triazole was carried out and then the title compound (40 mg, 0.09 mmol, yield: 12%) of white solid form was obtained by the same treatment procedure to that described in Example 9-43.

¹H NMR (400 MHz, DMSO-d₆); δ 12.16 (1H, s), 9.34 (1H, br), 7.59 (1H, br), 6.56 (1H, br), 5.18 (2H, s), 4.39 (2H, m), 4.24 (2H, m), 2.80 (2H, t), 1.67 (2H, m), 0.98 (3H, t)

### Example 9-54

### N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-3-yl}-acetamide

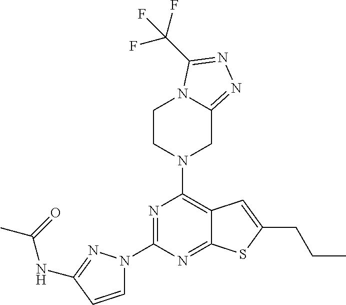

According to the same method to that described in Example 9-49, the title compound (10 mg, 0.02 mmol, yield: 3%) of white solid form was obtained by using the compound (0.25 g, 0.56 mmol) obtained from Example 9-52.

¹H NMR (400 MHz, CDCl₃); δ 8.18 (1H, d), 7.52 (1H, s), 7.17 (1H, d), 6.89 (1H, s), 5.25 (2H, s), 4.39 (2H, m), 4.28 (2H, m), 2.83 (2H, t), 1.74 (2H, m), 1.04 (3H, t)

### Example 9-55

### 7-[2-(4-Methyl-pyrazol-1-yl)-6-propyl-thieno[2,3-d]pyrimidin-4-yl]-3-trifluoromethyl-5,6,7,8-tetrahydro-[1,2,4]triazolo[4,3-a]pyrazine

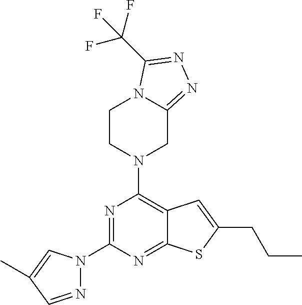

According to the same method to that described in Example 9-43, the reaction using the compound (1.0 g, 2.48 mmol) obtained from Preparation Example 1-1-3 and 4-methylpyrazole (285 mg, 3.48 mmol) instead of 1,2,4-triazole was carried out and then the title compound (860 mg, 1.91 mmol, yield: 77%) of white solid form was obtained by the same treatment procedure to that described in Example 9-43.

¹H NMR (400 MHz, CDCl₃); δ 8.26 (1H, s), 7.62 (1H, s), 7.01 (1H, s), 5.35 (2H, s), 4.42 (2H, br), 2.89 (2H, t), 1.81 (2H, m), 1.05 (3H, t)

### Example 9-56

### 1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-4-ylamine

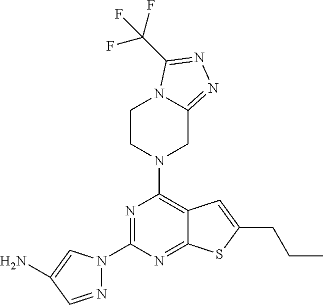

According to the same method to that described in Example 9-43, the reaction using the compound (1.0 g, 2.48 mmol) obtained from Preparation Example 1-1-3 and 4-amino-1H-pyrazole (289 mg, 3.48 mmol) instead of 1,2,4-triazole was carried out and then the title compound (100 mg, 0.22 mmol, yield: 9%) of white solid form was obtained by the same treatment procedure to that described in Example 9-43.

¹H NMR (400 MHz, DMSO-d₆); δ 7.91 (1H, s), 7.49 (1H, s), 7.36 (1H, s), 5.31 (2H, s), 4.41 (2H, m), 4.38 (2H, m), 4.32 (2H, br), 2.90 (2H, t), 1.71 (2H, m), 0.98 (3H, t)

### Example 9-57

### N-{1-[6-Propyl-4-(3-trifluoromethyl-5,6-dihydro-8H-[1,2,4]triazolo[4,3-a]pyrazin-7-yl)-thieno[2,3-d]pyrimidin-2-yl]-1H-pyrazol-4-yl}-acetamide

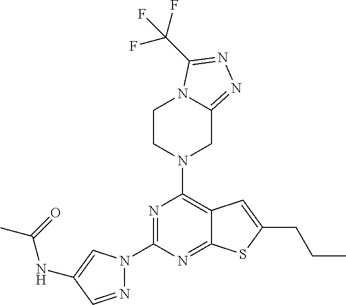

According to the same method to that described in Example 9-49, the title compound (20 mg, 0.04 mmol, yield: 18%) of white solid form was obtained by using the compound (0.10 g, 0.22 mmol) obtained from Example 9-56.

¹H NMR (400 MHz, DMSO-d₆); δ 10.20 (1H, s), 8.76 (1H, s), 7.79 (1H, s), 7.54 (1H, s), 5.34 (2H, s), 4.42 (2H, m), 4.40 (2H, m), 2.91 (2H, t), 2.04 (3H, s), 1.74 (2H, m), 0.98 (3H, t)

### Example 9-58

### [6-Propyl-4-(3-trifluoromethyl-5,6-dihydro-8H-[1,2,4]triazolo[4,3-a]pyrazin-7-yl)-thieno[2,3-d]pyrimidin-2-yl]-(1H-pyrazol-4-yl)-amine

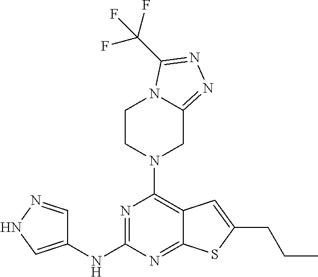

According to the same method to that described in Example 9-43, the reaction using the compound (1.0 g, 2.48 mmol) obtained from Preparation Example 1-1-3 and 4-amino-1H-pyrazole (289 mg, 3.48 mmol) instead of 1,2,4-triazole was carried out and then the title compound (75 mg, 0.17 mmol, yield: 7%) of white solid form was obtained by the same treatment procedure to that described in Example 9-43.

¹H NMR (400 MHz, DMSO-d₆); δ 12.45 (1H, br), 9.14 (1H, s), 7.84 (1H, br), 7.51 (1H, br), 7.24 (1H, s), 5.16 (2H, s), 4.41 (2H, m), 4.38 (2H, m), 2.81 (2H, t), 1.69 (2H, m), 0.98 (3H, t)

### Example 9-59

### 3-[4-(8-Oxo-3-trifluoromethyl-5,6-dihydro-8H-[1,2,4]triazolo[4,3-a]pyrazin-7-yl)-6-propyl-thieno[2,3-d]pyrimidin-2-yloxy]-propionic acid

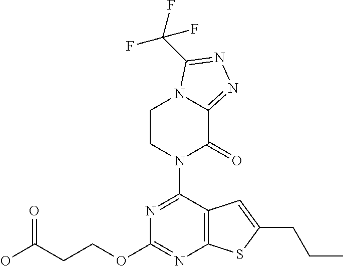

The compound (100 mg, 0.226 mmol) obtained from Example 3-10 was dissolved in acetonitrile 3 mL and pH 6.7 buffer solution 2 mL. 2,2,6,6-tetramethylpiperidin-1-oxy 10 mg, sodium chloride (61 mg, 0.678 mmol) and sodium hypochloride aqueous solution 0.05 mL were added thereto and stirred for 24 hours. Ethyl acetate 15 mL was added to the resulting mixture and washed two times with water 5 mL. After the organic layer was dried with anhydrous magnesium sulfate and distilled under reduced pressure, the title compound (22 mg, 21%) was obtained by column-chromatography using ethyl acetate.

¹H NMR (400 MHz, CDCl₃); δ 6.73 (1H, s), 4.70-4.63 (6H, m), 2.89-2.79 (4H, m), 1.77-1.68 (2H, m), 1.01-0.96 (3H, m)

### Example 9-60

### 7-{6-Propyl-2-[(S)-3-(pyrrolidine-1-carbonyl)-pyrrolidin-1-yl]-thieno[2,3-d]pyrimidin-4-yl}-3-trifluoromethyl-6,7-dihydro-5H-[1,2,4]triazolo[4,3-a]pyrazin-8-one

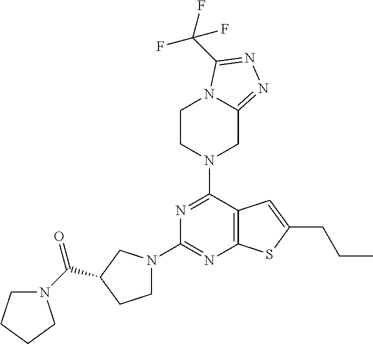

According to the same method to that described in Example 1-86, the title compound (15 mg, 11%) was obtained by using the compound (130 mg, 0.27 mmol) obtained from Example 1-57 and pyrrolidine (0.038 ml, 0.54 mmol).

¹H NMR (400 MHz, MeOD); δ 6.93 (1H, s), 5.05 (2H, s), 4.30-4.14 (4H, m), 3.75-3.26 (9H, m), 2.72-2.68 (2H, m), 2.17-2.04 (2H, m), 1.95-1.80 (4H, m), 1.78-1.57 (2H, m), 0.90 (3H, t)

### Example 9-61

### 3-[6-Propionyl-4-(3-trifluoromethyl-5,6-dihydro-8H-[1,2,4]triazolo[4,3-a]pyrazin-7-yl)-thieno[2,3-d]pyrimidin-2-yloxy]-propionic acid

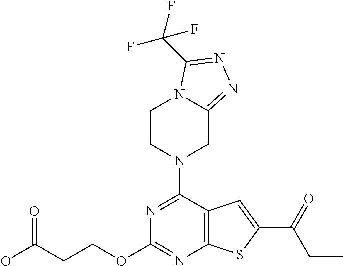

According to the same method to that described in Example 9-59, the title compound (35 mg, 33%) was obtained by using the compound (100 mg, 0.226 mmol) obtained from Example 3-10.

¹H NMR (400 MHz, CDCl₃); δ 8.04 (1H, s), 5.41 (2H, s), 4.67 (2H, t), 4.44 (4H, s), 3.06-3.00 (2H, m), 2.86-2.82 (2H, m), 1.27 (3H, t)

### Example 9-62

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid butylamide

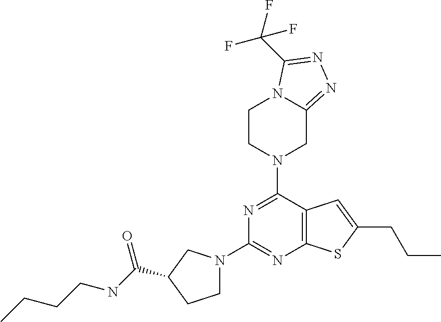

According to the same method to that described in Example 1-86, the title compound (60 mg, 53%) was obtained by using the compound (100 mg, 0.21 mmol) obtained from Example 1-57 and butylamine (0.031 ml, 0.42 mmol).

¹H NMR (400 MHz, CDCl₃); δ 6.76 (1H, s), 5.62 (2H, br), 5.22-5.12 (2H, m), 4.34-4.18 (4H, m), 3.90-3.89 (2H, m), 3.74-3.69 (1H, m), 3.59-3.49 (1H, m), 3.31-3.23 (2H, m), 2.98-2.91 (1H, m), 2.80-2.75 (2H, m), 2.32-2.22 (2H, m), 1.76-1.67 (2H, m), 1.54-1.47 (2H, m), 1.40-1.34 (2H, m), 1.01-0.91 (6H, m)

### Example 9-63

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid butyl-methyl-amide

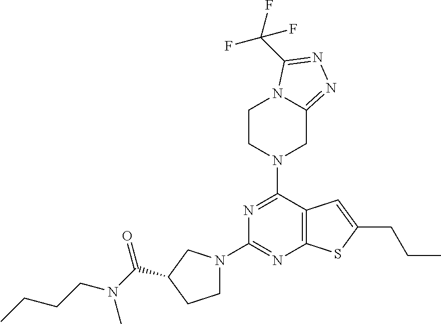

According to the same method to that described in Example 1-86, the title compound (41 mg, 36%) was obtained by using the compound (100 mg, 0.21 mmol) obtained from Example 1-57 and N-methylbutylamine (0.052 ml, 0.42 mmol).

¹H NMR (400 MHz, CDCl₃); δ 6.78 (1H, s), 5.20-5.11 (2H, m), 4.35-4.18 (4H, m), 3.91-3.57 (4H, m), 3.41-2.74 (8H, m), 2.31-2.15 (2H, m), 1.74-1.29 (6H, m), 1.01-0.91 (6H, m)

### Example 9-64

### (S)-1-[6-Propyl-4-(3-trifluoromethyl-5,6-dihydro-8H-[1,2,4]triazolo[4,3-a]pyrazin7-yl)-thieno[2,3-d]pyrimidin-2-yl]-pyrrolidine-3-carboxylic acid cyclopentylamide

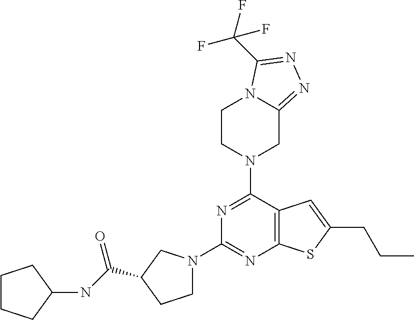

According to the same method to that described in Example 1-86, the title compound (46 mg, 40%) was obtained by using the compound (100 mg, 0.21 mmol) obtained from Example 1-57 and cyclopentylamine (0.042 ml, 0.42 mmol).

¹H NMR (400 MHz, CDCl₃); δ 6.76 (1H, s), 5.74 (2H, d), 5.21-5.11 (2H, m), 4.35-4.32 (2H, m), 4.25-4.18 (3H, m), 3.89-3.78 (2H, m), 3.71-3.67 (1H, m), 3.57-3.50 (1H, m), 2.95-2.90 (1H, m), 2.76 (2H, t), 2.29-2.18 (2H, m), 2.04-1.97 (2H, m), 1.74-1.58 (6H, m), 1.43-1.36 (2H, m), 0.99 (3H, t)

### Example 9-65

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid cyclohexylmethyl-amide

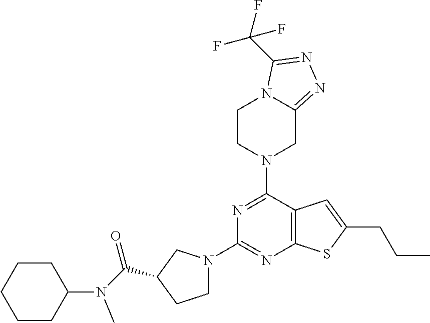

According to the same method to that described in Example 1-86, the title compound (96 mg, 79%) was obtained by using the compound (100 mg, 0.21 mmol) obtained from Example 1-57 and N-methylcyclohexylamine (0.049 ml, 0.42 mmol).

¹H NMR (400 MHz, CDCl₃); δ 6.77 (1H, s), 5.20-5.11 (2H, m), 4.45 (1H, br), 4.34 (2H, t), 4.19 (3H, t), 3.93-3.57 (4H, m), 3.34-3.30 (1H, m), 2.95 (1.6H, s), 2.84 (1.3H, s), 2.76 (2H, t), 2.34-2.12 (2H, m), 1.91-1.53 (8H, m), 1.45-1.34 (3H, m), 1.21-1.08 (1H, m), 0.99 (3H, t)

### Example 9-66

### N-Methyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide

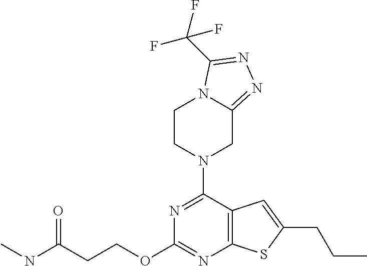

According to the same method to that described in Example 1-86, the title compound (19 mg, 60%) was obtained by using the compound (30 mg, 0.066 mmol) obtained from Example 3-11 and 2M methylamine (0.043 mL, 0.086 mmol) included in tetrahydrofuran.

¹H NMR (400 MHz, CDCl₃); δ 6.97 (1H, s), 6.69 (1H, br), 5.30 (2H, s), 4.62 (2H, t), 4.41-4.33 (4H, m), 2.86-2.80 (5H, m), 2.70-2.66 (2H, t), 1.80-1.71 (2H, m), 1.01 (3H, t)

### Example 9-67

### N,N-Dimethyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide

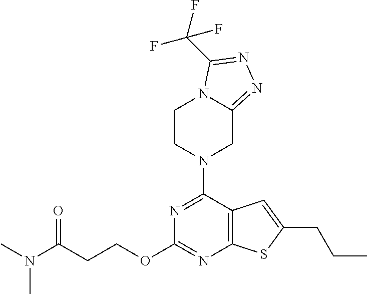

According to the same method to that described in Example 1-86, the title compound (45 mg, 42%) was obtained by using the compound (100 mg, 0.22 mmol) obtained from Example 3-11 and 2M dimethylamine (0.14 mL, 0.29 mmol) included in tetrahydrofuran.

¹H NMR (400 MHz, CDCl₃); δ 6.95 (1H, s), 5.29 (2H, s), 4.70 (2H, t), 4.39-4.31 (4H, m), 3.05 (2H, s), 2.96 (2H, s), 2.88-2.79 (4H, m), 1.79-1.70 (2H, m), 1.01 (3H, t)

### Example 9-68

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-1-pyrrolidin-1-yl-propan-1-one

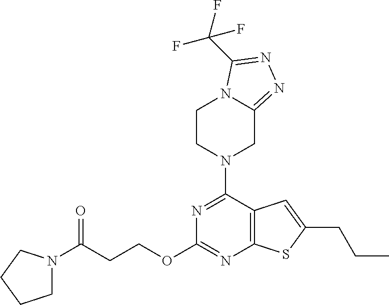

According to the same method to that described in Example 1-86, the title compound (70 mg, 65%) was obtained by using the compound (100 mg, 0.22 mmol) obtained from Example 3-11 and pyrrolidine (0.029 ml, 0.29 mmol).

¹H NMR (400 MHz, CDCl₃); δ 6.95 (1H, s), 5.29 (2H, s), 4.71 (2H, t), 4.39-4.31 (4H, m), 3.47 (4H, t), 2.84-2.79 (4H, m), 2.02-1.72 (6H, m), 1.01 (3H, t)

### Example 9-69

### N-Cyclopentyl-3-[6-propyl-4-(3-trifluoromethyl-5,6-dihydro-8H-[1,2,4]triazolo[4,3-a]pyrazin-7-yl)-thieno[2,3-d]pyrimidin-2-yloxy]-propionamide

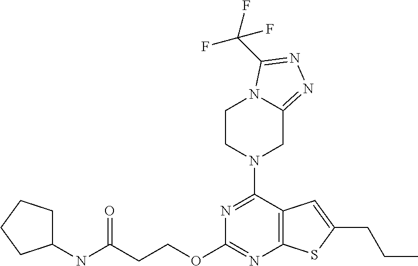

According to the same method to that described in Example 1-86, the title compound (70 mg, 61%) was obtained by using the compound (100 mg, 0.22 mmol) obtained from Example 3-11 and cyclopentylamine (0.042 ml, 0.42 mmol).

¹H NMR (400 MHz, CDCl₃); δ 6.95 (1H, s), 5.97-5.95 (1H, m), 5.29 (2H, s), 4.64 (2H, t), 4.38-4.32 (4H, m), 4.23-4.15 (1H, m), 2.85 (3H, t), 2.65 (3H, t), 2.01-1.92 (3H, m), 1.79-1.53 (6H, m), 1.42-1.34 (2H, m), 1.01 (3H, t)

### Example 9-70

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionamide

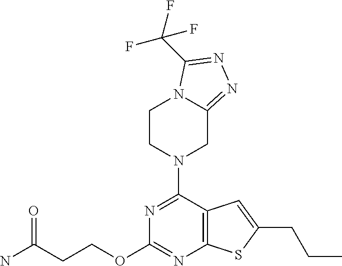

According to the same method to that described in Example 1-86, the title compound (32 mg, 61%) was obtained by using the compound (50 mg, 0.11 mmol) obtained from Example 3-11 and ammonium chloride (12 mg, 0.22 mmol).

¹H NMR (400 MHz, CDCl₃); δ 7.01 (1H, s), 5.31 (2H, s), 4.63 (2H, t), 4.41-4.34 (4H, m), 2.87-2.71 (4H, m), 1.81-1.68 (2H, m), 1.04-0.96 (3H, m)

### Example 9-71

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionitrile

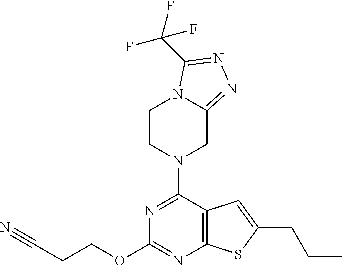

The compound (120 mg, 0.263 mmol) obtained from Example 9-70 was diluted with chloromethane 5 mL. Trifluoroacetic acid (74 μl, 0.527 mmol) was added thereto at 0° C. and stirred for 1 hour. The reaction mixture was diluted with ethyl acetate and washed with sodium hydrogen carbonate solution and brine. After the organic layer was dried with anhydrous magnesium sulfate and distilled under reduced pressure, the title compound (66 mg, 57%) was obtained by column-chromatography using 3:97 mixture of methanol and dichloromethane.

¹H NMR (400 MHz, CDCl₃); δ 6.98 (1H, s), 5.30 (2H, s), 4.62-4.59 (2H, m), 4.39-4.34 (4H, m), 3.00-2.68 (4H, m), 1.80-1.67 (2H, m), 1.04-0.95 (3H, m)

### Preparation Example 9-72-1

### N′-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carbonyl}-hydrazinecarboxylic acid tert-butyl ester

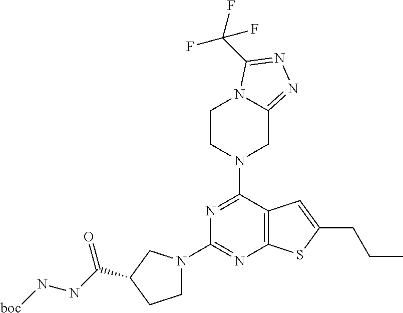

According to the same method to that described in Example 1-86, the title compound (70 mg, 28%) was obtained by using the compound (200 mg, 0.415 mmol) obtained from Example 1-57 and t-butyl carbazide (82 mg, 0.623 mmol).

¹H NMR (400 MHz, CDCl₃); δ 8.14 (1H, br), 6.76 (2H, s), 5.15-5.14 (2H, m), 4.34-4.17 (4H, m), 3.90-3.74 (3H, m), 3.58-3.56 (1H, m), 3.10-3.06 (1H, m), 2.76 (2H, t), 2.30-2.23 (2H, m), 1.73-1.68 (2H, m), 1.46 (9H, s), 0.99 (3H, t)

### Example 9-72

### Hydrochloric Acid Salt of

### (S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidine-3-carboxylic acid hydrazide

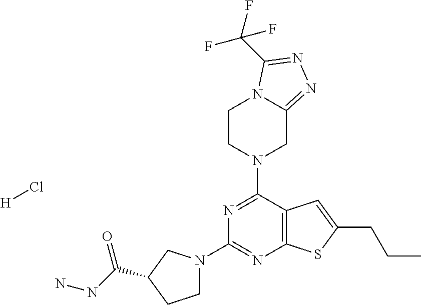

The compound (70 mg, 0.118 mmol) obtained from Preparation Example 9-72-1 was treated by the same method to that described in Example 1-4 to give the title compound (50 mg, 79%).

¹H NMR (400 MHz, DMSO); δ 11.28 (1H, s), 7.30 (1H, s), 5.22-5.20 (2H, m), 4.37-4.27 (4H, m), 3.70-3.17 (8H, m), 2.81-2.78 (2H, m), 2.33-2.13 (2H, m), 1.70-1.64 (2H, m), 0.95 (3H, t)

### Preparation Example 9-73-1

### N′-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionyl}-hydrazinecarboxylic acid tert-butyl ester

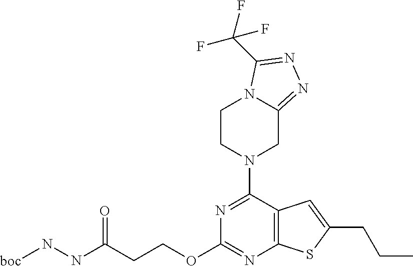

According to the same method to that described in Example 1-86, the title compound (90 mg, 48%) was obtained by using the compound (150 mg, 0.329 mmol) obtained from Example 3-11 and t-butyl carbazide (67 mg, 0.494 mmol).

¹H NMR (400 MHz, CDCl₃); δ 8.49 (1H, s), 6.94 (1H, s), 6.89 (1H, s) 5.28 (2H, s), 4.66 (2H, t), 4.37-4.32 (4H, m), 2.96-2.77 (4H, m), 1.82-1.69 (2H, m), 1.44 (9H, s), 1.03 (3H, t)

### Example 9-73

### Hydrochloric Acid Salt of

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propionic acid hydrazide

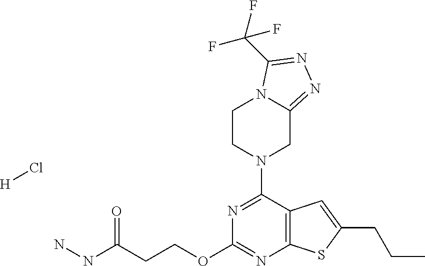

The compound (90 mg, 0.158 mmol) obtained from Preparation Example 9-73-1 was treated by the same method to that described in Example 1-4 to give the title compound (20 mg, 25%).

¹H NMR (400 MHz, MeOD); δ 7.61 (1H, s), 5.66 (2H, s), 5.20-4.95 (2H, m), 4.71-4.60 (4H, m), 2.99 (4H, br), 1.81 (2H, br), 1.06 (3H, br))

### Preparation Example 9-74-1

### (2-Hydroxy-propyl)-carbamic acid tert-butyl ester

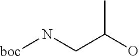

DL-1-amino-2-propanol (5 g, 66.57 mmol) was diluted with tetrahydrofuran 150 mL. Di t-butyl dicarbonate (18.9 g, 86.6 mmol) was added thereto and stirred for 24 hours at room temperature. The reaction mixture was distilled under reduced pressure, diluted with ethyl acetate and washed with water and brine. After the organic layer was dried with anhydrous magnesium sulfate and distilled under reduced pressure, the title compound (11 g, 63%) was obtained by column-chromatography using 1:4 mixture of hexane and ethyl acetate.

¹H NMR (400 MHz, CDCl₃); δ 4.97 (1H, br), 3.91-3.88 (1H, m), 3.29-3.24 (1H, m) 3.04-2.97 (1H, m), 2.48 (1H, br), 1.45 (9H, s), 1.18 (3H, d)

### Preparation Example 9-74-2

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid tert-butyl ester

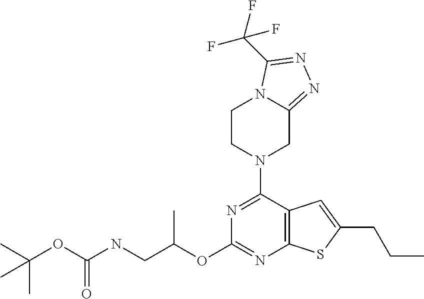

According to the same method to that described in Example 1-45, the title compound (2.48 g, 61%) was obtained by using the compound (3 g, 7.54 mmol) obtained from Preparation Example 1-1-3 and the compound (1.96 g, 11.18 mmol) obtained from Preparation Example 9-74-1.

¹H NMR (400 MHz, CDCl₃); δ 6.94 (1H, s), 5.30-5.25 (3H, m), 4.98 (1H, br), 4.37-4.34 (4H, m) 3.51-3.35 (2H, m), 2.83 (2H, t), 1.80-1.70 (2H, m), 1.42 (9H, s), 1.37 (3H, d), 1.01 (3H, t)

### Preparation Example 9-74-3

### Hydrochloric Acid Salt of

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propylamine

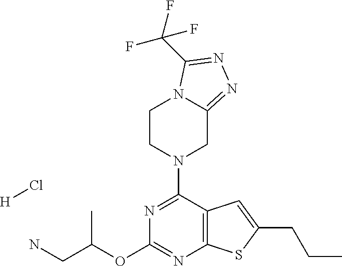

The compound (2.48 g, 4.58 mmol) obtained from Preparation Example 9-74-2 was treated by the same method to that described in Example 1-4 to give the title compound (2.19 g, 100%).

¹H NMR (400 MHz, DMSO); δ 8.37-8.28 (3H, m), 7.44 (1H, s), 5.42-5.38 (1H, m), 5.25 (2H, s) 4.44-4.28 (4H, m), 3.17-3.08 (2H, m), 2.87-2.83 (2H, m), 1.74-1.65 (2H, m), 1.36-1.31 (3H, m), 0.96 (3H, t)

### Example 9-74

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-formamide

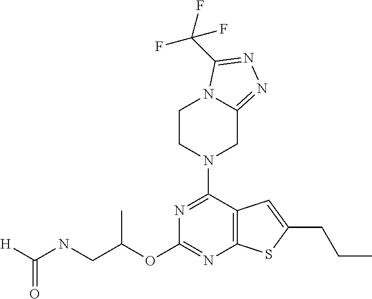

The title compound (22 mg, 11%) was obtained by treating the compound (200 mg, 0.42 mmol) obtained from Preparation Example 9-74-3 and formic acid (25 mg, 0.53 mmol) according to the same method to that described in Example 1-86.

¹H NMR (400 MHz, CDCl₃); δ 8.20 (2H, d), 6.95 (1H, s), 6.41 (1H, br), 5.37-5.16 (3H, m), 4.50-4.30 (4H, m), 3.75-3.69 (1H, m), 3.55-3.43 (1H, m), 2.85-2.83 (2H, m) 1.79-1.68 (2H, m), 1.39 (3H, d), 1.01 (3H, t)

### Preparation Example 9-75-1

### (2-Hydroxy-1-methyl-ethyl)-carbamic acid tert-butyl ester

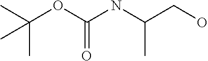

DL-1-amino-2-propanol (5 g, 66.57 mmol) was diluted with tetrahydrofuran 150 mL. Di t-butyl dicarbonate (18.9 g, 86.6 mmol) was added thereto and stirred for 24 hours at room temperature. The reaction mixture was distilled under reduced pressure, diluted with ethyl acetate and washed with water and brine. After the organic layer was dried with anhydrous magnesium sulfate and distilled under reduced pressure, the title compound (11 g, 63%) was obtained by column-chromatography using 1:4 mixture of hexane and ethyl acetate.

¹H NMR (400 MHz, CDCl₃); δ 4.71 (1H, br), 3.77 (1H, br), 3.66-3.61 (1H, m), 3.53-3.48 (1H, m) 2.81 (1H, br), 1.45 (9H, s), 1.16 (3H, d)

### Preparation Example 9-75-2

### {1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid tert-butyl ester

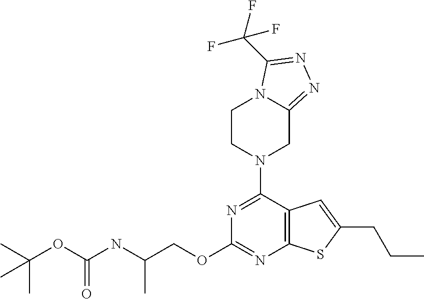

According to the same method to that described in Example 1-45, the title compound (2.9 g, 72%) was obtained by using the compound (3 g, 7.54 mmol) obtained from Preparation Example 1-1-3 and the compound (1.96 g, 11.18 mmol) obtained from Preparation Example 9-75-1.

¹H NMR (400 MHz, CDCl₃); δ 6.94 (1H, s), 5.31 (2H, s), 4.38-4.35 (5H, m), 4.28-4.24 (1H, m) 4.15-4.09 (1H, m), 2.83 (2H, t), 1.78-1.72 (2H, m), 1.43 (9H, s), 1.30-1.24 (3H, m), 1.01 (3H, t)

### Preparation Example 9-75-3

### Hydrochloric Acid Salt of

### 1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethylamine

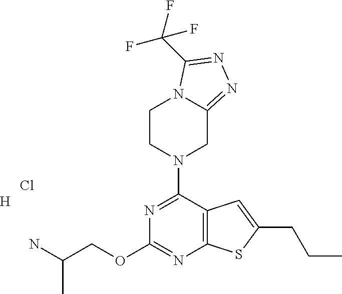

The title compound (2.56 g, 100%) was obtained by treating the compound (2.9 g, 5.35 mmol) obtained from Preparation Example 9-75-2 according to the same method to that described in Example 1-4.

¹H NMR (400 MHz, DMSO); δ 8.38 (1H, br), 7.45 (1H, s), 5.26 (2H, s), 4.50-4.29 (2H, m) 3.71-3.63 (1H, m), 2.85 (2H, t), 1.74-1.65 (2H, m), 1.32 (3H, d), 0.96 (3H, t)

### Example 9-75

### N-{1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide

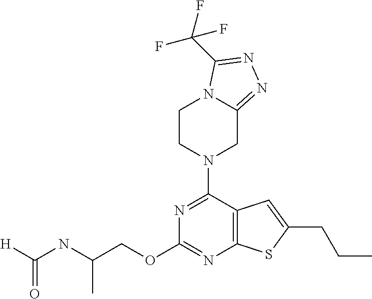

The title compound (59 mg, 30%) was obtained by treating the compound (200 mg, 0.42 mmol) obtained from Preparation Example 9-75-3 and formic acid (25 mg, 0.53 mmol) according to the same method to that described in Example 1-86.

¹H NMR (400 MHz, CDCl₃); δ 8.19-8.16 (1H, m), 6.95 (1H, s), 6.03 (1H, d), 5.35-5.27 (2H, m), 4.51-4.33 (7H, m), 2.85-2.82 (2H, m), 1.79-1.71 (2H, m), 1.35 (3H, d), 1.01 (3H, t)

### Example 9-76

### N-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-acetamide

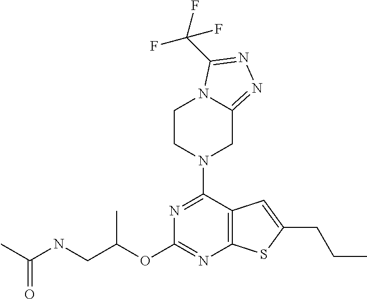

The title compound (148 mg, 73%) was obtained by treating the compound (200 mg, 0.42 mmol) obtained from Preparation Example 9-74-3 according to the same method to that described in Example 1-22.

¹H NMR (400 MHz, CDCl₃); δ 6.98 (1H, s), 6.54-6.51 (1H, m), 5.35-5.26 (3H, m), 4.38-4.32 (4H, m), 3.70-3.64 (1H, m), 3.47-3.49 (1H, m), 2.83 (2H, t) 1.98-1.97 (3H, m), 1.81-1.70 (2H, m), 1.39-1.30 (3H, m), 1.01 (3H, t)

### Example 9-77

### N-{1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

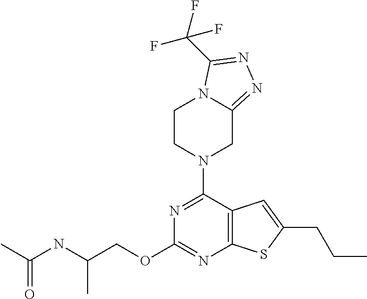

The title compound (139 mg, 68%) was obtained by treating the compound (200 mg, 0.42 mmol) obtained from Preparation Example 9-75-3 according to the same method to that described in Example 1-22.

¹H NMR (400 MHz, CDCl₃); δ 6.98 (1H, s), 6.46 (1H, d), 5.35-5.26 (2H, m), 4.40-4.22 (7H, m), 2.83 (2H, t), 1.99 (3H, s), 1.79-1.70 (2H, m), 1.31-1.30 (m, 3H), 1.01 (3H, t)

### Preparation Example 9-78-1

### (2-Hydroxy-1,1-dimethyl-ethyl)-carbamic acid tert-butyl ester

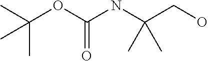

The title compound (4.8 g, 75%) was obtained by treating 2-amino-2-methyl-1-propanol (3 g, 33.65 mmol) according to the same method to that described in Preparation Example 9-75-1.

¹H NMR (400 MHz, CDCl₃); δ 4.73 (1H, br), 4.15-4.10 (1H, br), 3.58-3.57 (2H, m), 1.43 (9H, s), 1.28-1.25 (6H, m)

### Preparation Example 9-78-2

### {1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid tert-butyl ester

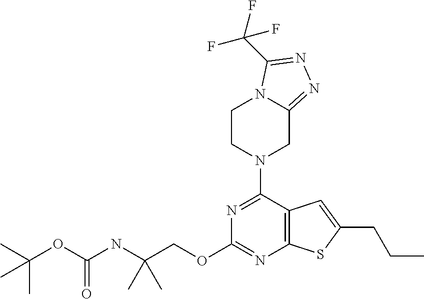

According to the same method to that described in Example 1-45, the title compound (2.44 g, 59%) was obtained by using the compound (3 g, 7.54 mmol) obtained from Preparation Example 1-1-3 and the compound (2.11 g, 11.18 mmol) obtained from Preparation Example 9-78-1.

¹H NMR (400 MHz, DMSO); δ 7.39 (1H, s), 6.62 (1H, br), 5.22 (2H, s), 4.39-4.28 (6H, m) 2.84 (2H, t), 1.73-1.64 (2H, m), 1.34 (9H, s), 1.27 (6H, s), 0.96 (3H, t)

### Preparation Example 9-78-3

### Hydrochloric Acid Salt of

### 1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethylamine

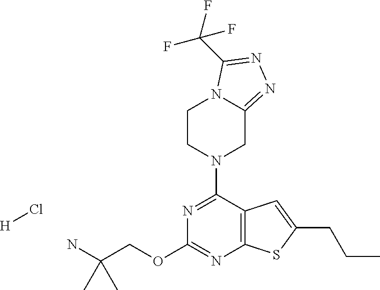

The title compound (2.16 g, 100%) was obtained by treating the compound (2.44 g, 4.39 mmol) obtained from Preparation Example 9-78-2 according to the same method to that described in Example 1-4.

¹H NMR (400 MHz, DMSO); δ 8.38 (1H, s), 7.45 (1H, s), 5.26 (2H, s), 4.44-4.33 (6H, m) 2.85 (2H, t), 1.75-1.65 (2H, m), 1.37 (6H, s), 0.96 (3H, t)

### Example 9-78

### N-{1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide

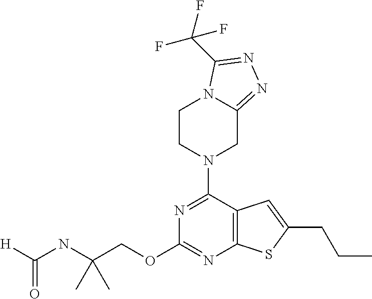

The title compound (177 mg, 60%) was obtained by treating the compound (300 mg, 0.61 mmol) obtained from Preparation Example 9-78-3 and formic acid (84 mg, 1.83 mmol) according to the same method to that described in Example 1-86.

¹H NMR (400 MHz, CDCl₃); δ 6.79 (1H, s), 5.16 (2H, s), 5.08 (1H, s), 4.34-4.32 (2H, m), 4.21-4.18 (2H, m), 3.71 (2H, s), 2.76 (2H, t), 1.75-1.66 (2H, m), 1.39 (1H, s), 1.01 (3H, t)

### Example 9-79

### N-{1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

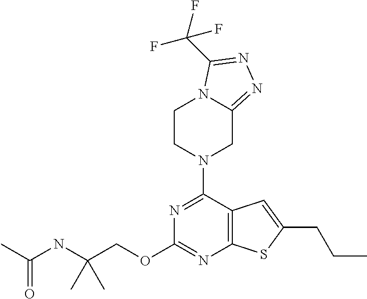

The title compound (58 mg, 28%) was obtained by treating the compound (200 mg, 0.41 mmol) obtained from Preparation Example 9-78-3 according to the same method to that described in Example 1-22.

¹H NMR (400 MHz, CDCl₃); δ 6.99 (1H, s), 6.31 (1H, s), 5.32 (2H, s), 4.46 (2H, s), 4.41-4.37 (4H, m), 2.83 (2H, t), 1.92 (3H, s), 1.80-1.70 (2H, m), 1.47 (6H, s), 1.01 (3H, t)

### Example 9-80

### {2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid methyl ester

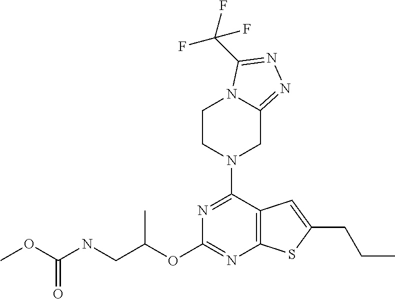

The compound (200 mg, 0.42 mmol) obtained from Preparation Example 9-78-3 was diluted with dichloromethane 5 mL. Triethylamine (0.3 mL, 0.21 mmol) and methylchloroformate (80 μl, 0.84 mmol) was added thereto at 0° C. and stirred for 3 hours. The reaction mixture was diluted with ethyl acetate and washed with water and brine. After the organic layer was dried with anhydrous magnesium sulfate and distilled under reduced pressure, the title compound (119 mg, 57%) was obtained by column-chromatography using 5:95 mixture of methanol and dichloromethane.

¹H NMR (400 MHz, CDCl₃); δ 6.95 (1H, s), 5.46-5.10 (4H, m), 4.39-4.30 (4H, m), 3.64 (3H, s), 3.58-3.36 (2H, m), 2.83 (2H, t), 1.80-1.70 (2H, m), 1.39-1.31 (3H, m), 1.01 (3H, t)

### Example 9-81

### {1-Methyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester

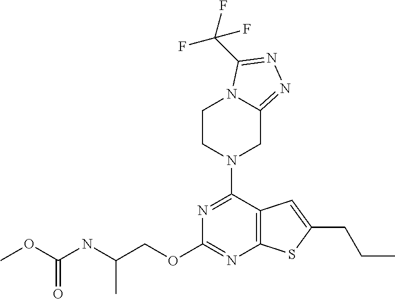

The title compound (88 mg, 42%) was obtained by treating the compound (200 mg, 0.42 mmol) obtained from Preparation Example 9-75-3 according to the same method to that described in Example 9-80.

¹H NMR (400 MHz, CDCl₃); δ 6.95 (1H, s), 5.34-5.26 (2H, m), 5.16 (1H, br), 4.39-4.30 (6H, m), 4.13 (1H, br), 3.65-3.63 (3H, m), 2.83 (2H, t), 1.80-1.70 (2H, m), 1.31 (3H, d), 1.01 (3H, t)

### Example 9-82

### {1,1-Dimethyl-2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester

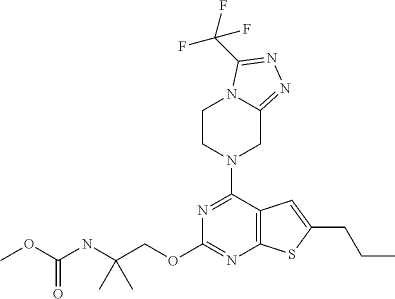

The title compound (38 mg, 18%) was obtained by treating the compound (200 mg, 0.42 mmol) obtained from Preparation Example 9-78-3 according to the same method to that described in Example 9-80.

¹H NMR (400 MHz, CDCl₃); δ 6.94 (1H, s), 5.30 (2H, s), 5.16 (1H, s), 4.40-4.30 (6H, m), 3.59 (3H, s), 2.85-2.79 (2H, m), 1.80-1.70 (2H, m), 1.45 (6H, s), 1.01 (3H, t)

### Example 9-83

### 7-[2-(2-Fluoro-ethoxy)-6-propyl-thieno[2,3-d]pyrimidin-4-yl]-3-trifluoromethyl-5,6,7,8-tetrahydro-[1,2,4]triazolo[4,3-a]pyrazine

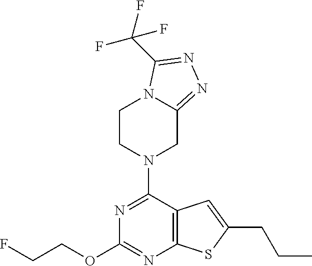

According to the same method to that described in Example 1-45, the title compound (144 mg, 27%) was obtained by using the compound (500 mg, 1.24 mmol) obtained from Preparation Example 1-1-3 and 2-fluoroethanol (119 mg, 1.86 mmol).

¹H NMR (400 MHz, CDCl₃); δ 6.95 (1H, s), 5.31 (2H, s), 4.85-4.83 (1H, m), 4.73-4.71 (1H, m), 4.68-4.66 (1H, m), 4.61-4.59 (1H, m), 4.38-4.32 (4H, m), 2.86-2.82 (2H, t), 1.80-1.70 (2H, m), 1.02 (3H, t)

### Example 9-84

### 7-[6-Propyl-2-(2,2,2-trifluoro-ethoxy)-thieno[2,3-d]pyrimidin-4-yl]-3-trifluoromethyl-5,6,7,8-tetrahydro-[1,2,4]triazolo[4,3-a]pyrazine

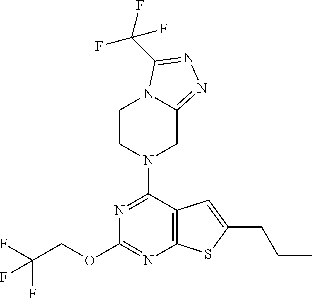

According to the same method to that described in Example 1-45, the title compound (240 mg, 42%) was obtained by using the compound (500 mg, 1.24 mmol) obtained from Preparation Example 1-1-3 and 2,2,2-trifluoroethanol (0.2 mL, 1.86 mmol).

¹H NMR (400 MHz, CDCl₃); δ 6.98 (1H, s), 5.33 (2H, s), 4.84-4.78 (2H, m), 4.37-4.36 (4H, m), 2.87-2.83 (2H, m), 1.79-1.73 (2H, m), 1.02 (3H, t)

### Example 9-85

### 1-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-pyrrolidin-2-one

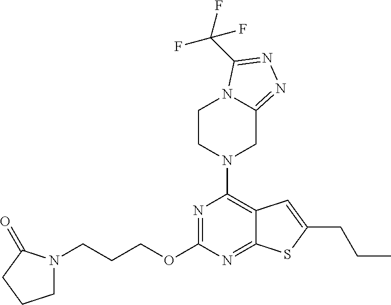

According to the same method to that described in Example 1-45, the title compound (1.09 g, 86%) was obtained by using the compound (1 g, 2.48 mmol) obtained from Preparation Example 1-1-3 and 1-(3-hydroxypropyl)-2-pyrrolidone (390 mg, 2.73 mmol).

¹H NMR (400 MHz, CDCl₃); δ 6.93 (1H, s), 5.30 (2H, s), 4.39 (4H, m), 4.32 (2H, m), 3.46 (4H, m), 2.83 (2H, t), 2.38 (2H, t), 2.08 (4H, m), 1.76 (2H, m), 1.03 (3H, t)

### Preparation Example 9-86-1

### (3-Hydroxy-propyl)-carbamic acid tert-butyl ester

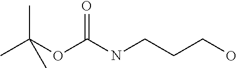

Amino-1-propanol (1 g, 13.3 mmol) and di t-butyl dicarbonate (2.9 g, 13.3 mmol) were diluted with dichloromethane 10 mL and stirred for 2 hours at room temperature. After the reaction mixture was distilled under reduced pressure, the title compound (2.26 g, 97%) was obtained by column-chromatography using 1:1 mixture of hexane and acetate.

¹H NMR (400 MHz, CDCl₃); δ 3.66 (2H, m), 3.29 (2H, m), 2.89 (1H, m), 1.66 (2H, m), 1.45 (2H, m)

### Preparation Example 9-86-2

### {3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid tert-butyl ester

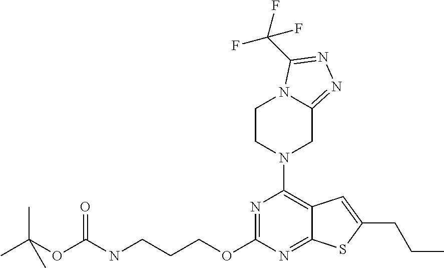

According to the same method to that described in Example 1-45, the title compound (3 g, 58%) was obtained by using the compound (4.59 g, 11.4 mmol) obtained from Preparation Example 1-1-3 and the compound (2.2 g, 12.5 mmol) obtained from Preparation Example 9-86-1.

MS (M+1): 456.7

### Preparation Example 9-86-3

### Hydrochloric Acid Salt of

### 3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propylamine

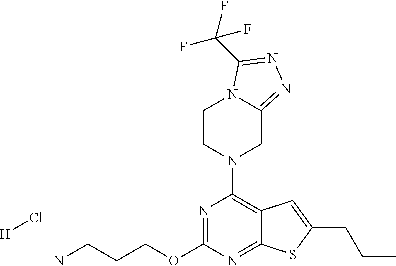

The title compound (1.0 g, 96%) was obtained by treating the compound (1 g, 2.19 mmol) obtained from Preparation Example 9-86-2 according to the same method to that described in Example 1-4.

MS (M+1): 478.9

### Example 9-86

### {3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-carbamic acid methyl ester

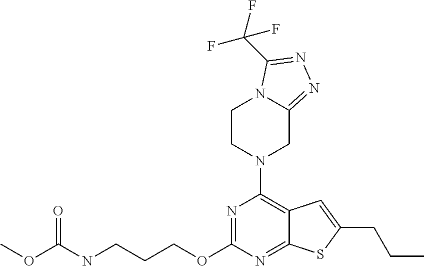

The compound (100 mg, 0.21 mmol) obtained from Preparation Example 9-86-3, methyl chloroformate (0.016 mL, 0.21 mmol) and triethylamine were diluted with dichloromethane 3 mL and stirred for 16 hours at room temperature. After the reaction mixture was distilled under reduced pressure, the title compound (56 mg, 54%) was obtained by column-chromatography using a mixture of methanol and dichloromethane.

¹H NMR (400 MHz, CDCl₃); δ 6.94 (1H, s), 5.30 (2H, s), 4.45 (2H, t), 4.38 (2H, t), 4.32 (2H, t), 3.67 (3H, s) 3.38 (2H, m), 2.83 (2H, t), 2.38 (2H, t), 2.02 (2H, m), 1.75 (2H, m), 1.03 (3H, t)

### Example 9-87

### N-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-acetamide

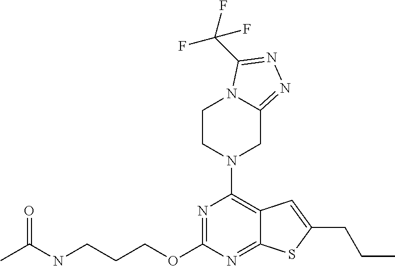

According to the same method to that described in Example 1-105, the title compound (20 mg, 24%) was obtained by using the compound (80 mg, 0.165 mmol) obtained from Preparation Example 9-86-3 and acetic anhydride (0.016 mL, 0.165 mmol).

¹H NMR (400 MHz, CDCl₃); δ 6.94 (1H, s), 5.30 (2H, s), 4.45 (2H, t), 4.36 (2H, t), 4.32 (2H, t), 3.42 (2H, m), 2.85 (2H, t), 2.02 (5H, m), 1.85 (2H, m), 1.03 (3H, t)

### Preparation Example 9-88-1

### (4-Hydroxy-butyl)-carbamic acid tert-butyl ester

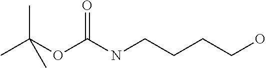

According to the same method to that described in Preparation Example 9-86-1, the title compound (2.05 g, 97%) was obtained by using 4-amino-1-butanol (1 g, 11.2 mmol) and t-butyl dicarbonate (2.45 g, 11.2 mmol).

¹H NMR (500 MHz, CDCl₃); δ 3.66 (2H, m), 3.15 (2H, m), 1.58 (4H, m), 1.43 (9H, s)

### Preparation Example 9-88-2

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-carbamic acid tert-butyl ester

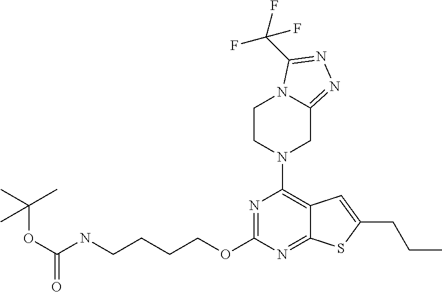

According to the same method to that described in Example 1-45, the title compound (900 mg, 38%) was obtained by using the compound (1.89 g, 4.7 mmol) obtained from Preparation Example 1-1-3 and the compound (980 mg, 5.18 mmol) obtained from Preparation Example 9-88-1.

¹H NMR (400 MHz, CDCl₃); δ 6.94 (1H, s), 5.30 (2H, s), 4.59 (2H, t), 4.15 (2H, t), 3.19 (2H, m), 3.67 (3H, s) 2.84 (2H, t), 1.86 (2H, m), 1.78 (2H, m), 1.67 (2H, m), 1.43 (9H, s) 1.03 (3H, t)

### Preparation Example 9-88-3

### Hydrochloric Acid Salt of

### 4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butylamine

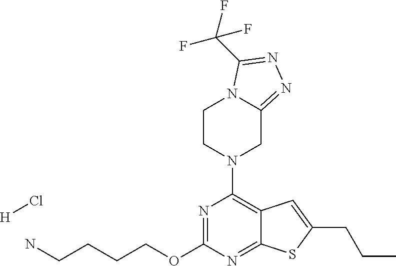

The title compound (765 mg, 96%) was obtained by treating the compound (900 mg, 1.62 mmol) obtained from Preparation Example 9-88-2 according to the same method to that described in Example 1-4.

MS (M+1): 491.97

### Example 9-88

### {4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-carbamic acid methyl ester

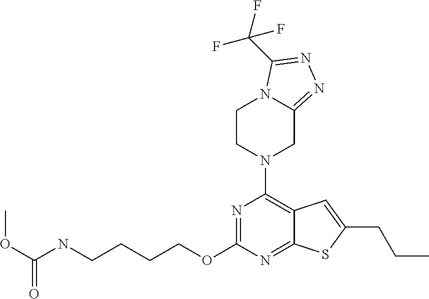

According to the same method to that described in Example 9-86, the title compound (50 mg, 49%) was obtained by using the compound (100 mg, 0.20 mmol) obtained from Preparation Example 9-88-3 and methyl chloroformate (0.015 mL, 0.20 mmol).

¹H NMR (400 MHz, CDCl₃); δ 6.94 (1H, s), 5.29 (2H, s), 4.37 (4H, m), 4.32 (2H, t), 3.66 (3H, s), 3.27 (2H, m), 2.85 (2H, t), 1.85 (2H, m), 1.76 (2H, m), 1.03 (3H, t)

### Example 9-89

### N-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-acetamide

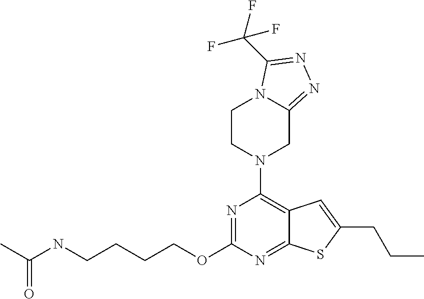

According to the same method to that described in Example 1-105, the title compound (45 mg, 47%) was obtained by using the compound (100 mg, 0.20 mmol) obtained from Preparation Example 9-88-3 and acetic anhydride (0.016 mL, 0.20 mmol).

¹H NMR (500 MHz, CDCl₃); δ 6.93 (1H, s), 5.28 (2H, s), 4.38 (4H, m), 4.36 (2H, t), 3.34 (2H, m), 2.83 (2H, t), 1.97 (3H, s), 1.73 (2H, m), 1.71 (4H, m) 1.03 (3H, t)

### Example 9-90

### N-{3-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-propyl}-formamide

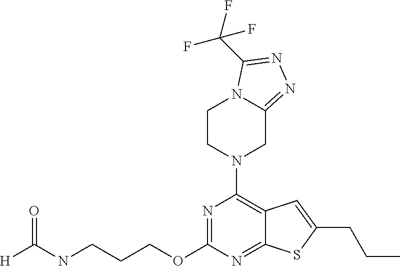

According to the same method to that described in Example 1-86, the title compound (10 mg, 11%) was obtained by using the compound (100 mg, 0.21 mmol) obtained from Preparation Example 9-86-3 and formic acid (0.009 mL, 0.23 mmol).

¹H NMR (400 MHz, CDCl₃); δ 8.20 (1H, s) 6.94 (1H, s), 5.29 (2H, s), 4.47 (2H, t), 4.37 (2H, t), 4.33 (2H, t), 3.49 (2H, m), 2.85 (2H, t), 2.05 (2H, m), 1.76 (2H, m), 1.03 (3H, t)

### Example 9-91

### N-{4-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-butyl}-formamide

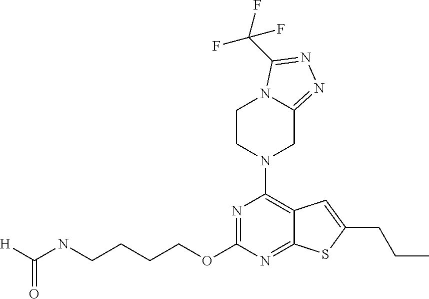

According to the same method to that described in Example 1-86, the title compound (10 mg, 10.4%) was obtained by using the compound (100 mg, 0.20 mmol) obtained from Preparation Example 9-86-3 and formic acid (0.009 mL, 0.20 mmol).

¹H NMR (400 MHz, CDCl₃); δ 8.19 (1H, s), 6.93 (1H, s), 5.30 (2H, s), 4.39 (4H, m), 4.32 (2H, t), 3.40 (2H, m), 2.83 (2H, t), 1.87 (2H, m), 1.74 (4H, m), 1.03 (3H, t)

### Example 9-92

### 7-[6-Propyl-2-(3-pyrrol-1-yl-propoxy)-thieno[2,3-d]pyrimidin-4-yl]-3-trifluoromethyl-5,6,7,8-tetrahydro-[1,2,4]triazolo[4,3-a]pyrazine

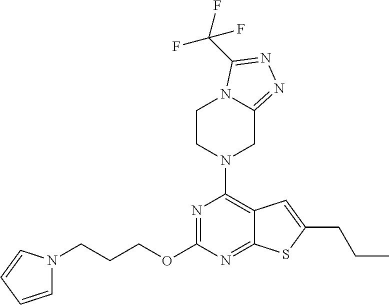

According to the same method to that described in Example 1-45, the title compound (100 mg, 30%) was obtained by using the compound (290 mg, 0.72 mmol) obtained from Preparation Example 1-1-3 and 1-(3-hydroxypropyl)-pyrrole (390 mg, 0.8 mmol).

¹H NMR (400 MHz, CDCl₃); δ 6.94 (1H, s), 6.68 (2H, m), 6.13 (2H, m) 5.29 (2H, s), 4.32 (6H, m), 4.12 (2H, t), 3.46 (4H, m), 2.85 (2H, t), 2.25 (2H, m), 2.08 (4H, m), 1.79 (2H, m), 1.03 (3H, t)

### Preparation Example 9-93-1

### [2-(tert-Butyl-dimethyl-silanyloxy)-ethyl]-methyl-carbamic acid tert-butyl ester

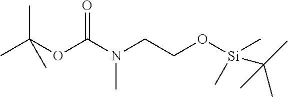

[2-(t-butyl-dimethyl-silanyloxy)-ethyl]-carbamic acid t-butyl ester (10 g, 36 mmol) was diluted in dimethyl formamide. Sodium hydride (2.16 g, 54 mmol) was added thereto at 0° C. and stirred for 30 minutes. Then, iodomethane (2.5 mL, 40 mmol) was added thereto at 0° C. and stirred. After the reaction mixture was distilled under reduced pressure, the title compound (7.2 g, 70%) was obtained by column-chromatography using 1:1 mixture of hexane and acetate.

¹H NMR (400 MHz, CDCl₃); δ 3.65 (2H, m), 3.25 (2H, m), 1.56 (9H, s), 0.84 (2H, s), 0 (6H, s),

### Preparation Example 9-93-2

### (2-Hydroxy-ethyl)-methyl-carbamic acid tert-butyl ester

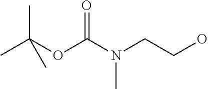

After the compound (7.2 g, 25 mmol) was treated by the same method to that described in Example 1-4, the title compound (2.8 g, 70%) was obtained by using t-butyl dicarbonate (11.5 g, 25 mmol) according to the same method to that described in Preparation Example 9-86-1.

¹H NMR (400 MHz, CDCl₃); δ 3.15 (2H, d), 3.09 (2H, d), 2.93 (3H, s), 1.62 (9H, s)

### Preparation Example 9-93-3

### Methyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid tert-butyl ester

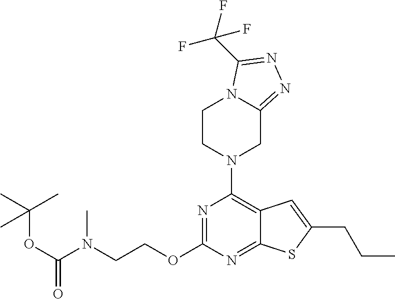

The title compound (3.8 g, 60%) was obtained by using the compound (1.9 g, 11.7 mmol) obtained from Preparation Example 9-93-2 and the compound (4.3 g, 10.7 mmol) obtained from Preparation Example 1-1-3.

¹H NMR (400 MHz, CDCl₃); δ 6.95 (1H, s), 5.31 (2H, s), 4.49 (2H, m), 4.37 (4H, m), 3.63 (2H, m), 2.97 (3H, s), 2.83 (2H, t), 1.76 (2H, m), 1.47 (9H, s), 1.03 (3H, t)

### Preparation Example 9-93-4

### Hydrochloric acid salt of Methyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amine

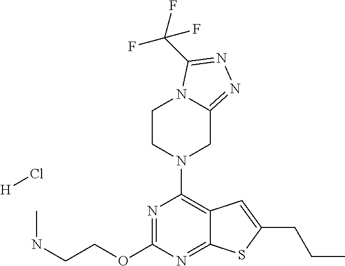

The compound (3.8 g, 7.0 mmol) obtained from Preparation Example 9-93-3 was treated by the same method to that described in Example 1-4 to give the title compound (2.67 g, 80%).

MS (M+1): 478.94

### Example 9-93

### N-Methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide

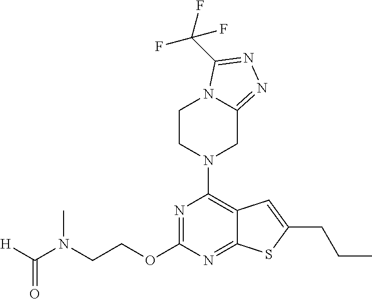

According to the same method to that described in Example 1-86, the title compound (5 mg, 25%) was obtained by using the compound (22 mg, 0.043 mmol) obtained from Preparation Example 9-93-4 and formic acid (0.009 mL, 0.052 mmol).

¹H NMR (400 MHz, CDCl₃); δ 8.08 (1H, d) 6.95 (1H, s), 5.31 (2H, s), 4.52 (4H, m), 4.33 (4H, m), 3.77 (1H, t), 3.67 (1H, t), 3.01 (1H, d), 2.83 (2H, t), 1.76 (2H, m), 1.03 (3H, t)

### Preparation Example 9-94-1

### (S)-3-Ethoxycarbonylamino-pyrrolidine-1-carboxylic acid benzyl ester

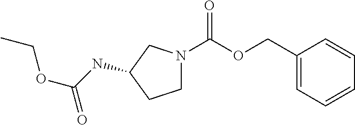

The title compound (2 g, 9.9 mmol) obtained by treatment of (S)-3-t-buthoxycarbonyl amino-pyrrolidine-1-carboxylic acid benzyl ester according to the same method to that described in Example 1-4, was diluted with tetrahydrofuran. Then, the reaction mixture was stirred with ethyl bromoacetate (1.6 g, 9.9 mmol) and triethylamine. After the reaction mixture was distilled under reduced pressure, the title compound (1.52 g, 70%) was obtained by column-chromatography using 1:2 mixture of hexane and acetate.

MS (M+1): 306.36

### Preparation Example 9-94-2

### (S)-3-[tert-Butoxycarbonyl-(2-hydroxy-ethyl)-amino]-pyrrolidine-1-carboxylic acid (E)-((Z)-2-propenyl)-penta-2,4-dienyl ester

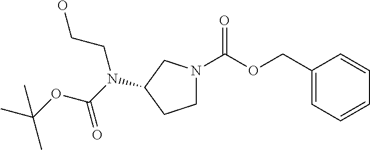

The compound (1.52 g, 4.96 mmol) obtained from Preparation Example 9-94-1 was diluted in tetrahydrofuran and stirred with lithium borohydride (2.0 M in tetrahydrofuran) (3.72 mL, 7.44 mmol.) at 0° C. According to the same method to that described in Preparation Example 9-86-1, the title compound (850 mg, 54%) was obtained by using t-butyl dicarbonate (957 mg, 4.38 mmol).

¹H NMR (400 MHz, CDCl₃); δ 7.36 (5H, m), 5.13 (2H, s), 4.50 (1H, m), 3.69 (5H, m), 3.35 (5H, m), 1.46 (9H, s)

### Preparation Example 9-94-3

### (2-Hydroxy-ethyl)-(S)-pyrrolidin-3-yl-carbamic acid tert-butyl ester

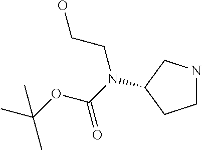

The compound (850 mg, 1.37 mmol) obtained from Preparation Example 9-94-2 was diluted in methanol and stirred with Pd/C (palladium on charcoal) (85 mg, 0.137 mmol) for hydrogenation. After removing Pd/C, the title compound (400 mg, 75%) was obtained by distillation of the reaction mixture under reduced pressure.

MS (M+1): 231.31

### Preparation Example 9-94-4

### (2-Hydroxy-ethyl)-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid tert-butyl ester

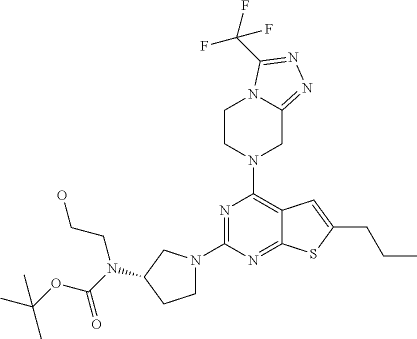

According to the same method to that described in Example 1-1, the compound (400 mg, 1.73 mmol) obtained from Preparation Example 9-94-3 was diluted in n-butanol with the compound (699 mg, 1.73 mmol) obtained from Preparation Example 1-1-3 and triethylamine, and stirred for 16 hours at 120° C. with reflux. The title compound (720 mg, 72%) was obtained by distillation of the reaction mixture under reduced pressure.

¹H NMR (400 MHz, CDCl₃); δ 6.77 (1H, s), 5.23 (2H, s), 5.17 (1H, m), 4.68 (2H, m), 4.19 (2H, m), 3.79 (4H, m), 3.43 (4H, m), 2.78 (2H, t), 2.18 (1H, m), 1.75 (2H, q), 1.69 (9H, s), 1.01 (3H, t)

### Preparation Example 9-94-5

### Hydrochloric Acid Salt of

### 2-{(S)-1-[6-Propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-ylamino}-ethanol

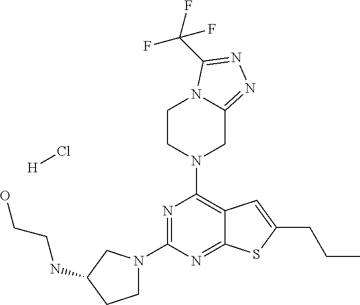

The title compound (600 mg, 94%) was obtained by treating the compound (720 mg, 1.2 mmol) obtained from Preparation Example 9-94-4 according to the same method to that described in Example 1-4.

MS (M+1): 534.02

### Example 9-94

### (2-Hydroxy-ethyl)-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-carbamic acid methyl ester

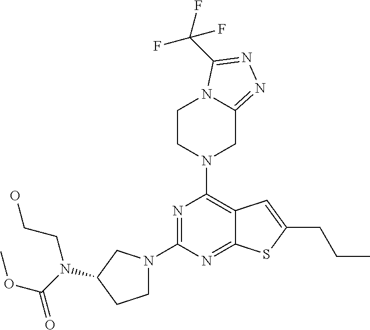

According to the same method to that described in Example 9-86, the title compound (20 mg, 20%) was obtained by using the compound (100 mg, 0.19 mmol) obtained from Preparation Example 9-94-5 and methyl chloroformate (0.015 mL, 0.19 mmol).

¹H NMR (400 MHz, CDCl₃); δ 6.98 (1H, s), 5.17 (2H, s), 4.68 (1H, m), 4.33 (2H, m), 4.20 (2H, m), 3.77 (7H, m), 3.46 (4H, m), 2.76 (2H, t), 2.18 (2H, m), 1.73 (2H, m), 0.99 (3H, t)

### Example 9-95

### Methyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester

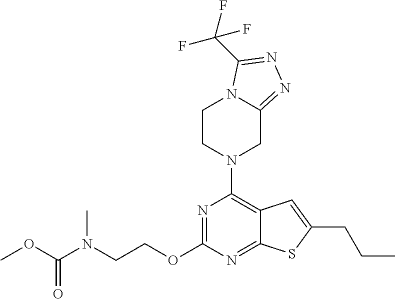

According to the same method to that described in Example 9-86, the title compound (10 mg, 10%) was obtained by using the compound (100 mg, 0.19 mmol) obtained from Preparation Example 9-93-4 and methyl chloroformate (0.018 mL, 0.19 mmol).

¹H NMR (500 MHz, CDCl₃); δ 6.93 (1H, s), 5.29 (2H, s), 4.37 (2H, m), 4.35 (4H, m), 3.68 (3H, s), 3.65 (2H, m), 3.01 (3H, d), 2.83 (2H, t), 1.76 (2H, m), 1.02 (3H, t)

### Example 9-96

### N-(2-Hydroxy-ethyl)-N-{(S)-1-[6-propyl-4-(3-trifluoromethyl-5,6-dihydro-8H-[1,2,4]triazolo[4,3-a]pyrazin-7-yl)-thieno[2,3-d]pyrimidin-2-yl]-pyrrolidin-3-yl}-acetamide

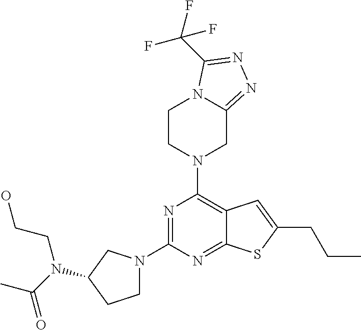

According to the same method to that described in Example 1-105, the title compound (10 mg, 10%) was obtained by using the compound (100 mg, 0.20 mmol) obtained from Preparation Example 9-94-5 and acetic anhydride (0.016 mL, 0.20 mmol).

¹H NMR (500 MHz, CDCl₃); δ 6.79 (1H, s), 5.20 (2H, s), 4.50 (2H, m), 4.33 (4H, m), 3.89 (2H, m), 3.50 (4H, m), 2.78 (2H, t), 2.76 (2H, t), 2.17 (3H, m), 2.03 (2H, m), 1.73 (2H, m), 1.02 (3H, t)

### Example 9-97

### N-Methyl-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

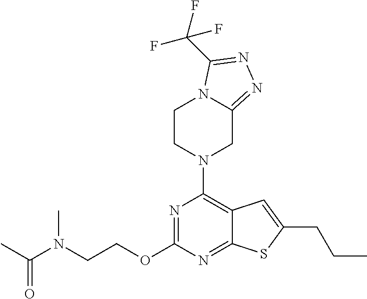

According to the same method to that described in Example 1-105, the title compound (10 mg, 10%) was obtained by using the compound (100 mg, 0.20 mmol) obtained from Preparation Example 9-93-4 and acetic anhydride (0.018 mL, 0.20 mmol).

¹H NMR (500 MHz, CDCl₃); δ 6.93 (1H, s), 5.28 (2H, s), 4.49 (2H, m), 4.33 (4H, m), 3.74 (2H, m), 3.65 (2H, m), 3.05 (3H, d), 2.83 (2H, m), 2.05 (3H, m) 1.73 (2H, m), 1.02 (3H, t)

### Preparation Example 9-98-1

### [2-(tert-Butyl-dimethyl-silanyloxy)-ethyl]-(2-hydroxy-ethyl)-carbamic acid tert-butyl ester

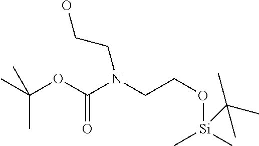

The title compound (9.6 g, 99%) obtained by using di-ethanol amine (5 g, 47 mmol) and t-butyl dicarbonate (10.25 g, 47 mmol) according to the same method to that described in Preparation Example 9-86-1, was diluted in dichloromethane. Then, the reaction mixture was stirred with t-butyl dimethylsilylchloride (7.1 g, 47 mmol) and imidazole (6.4 g, 94 mmol) at 0° C. After the reaction mixture was distilled under reduced pressure, the title compound (12 g, 80%) was obtained by column-chromatography using 1:1 mixture of hexane and acetate.

MS (M+1): 320.52

### Preparation Example 9-98-2

### [2-(tert-Butyl-dimethyl-silanyloxy)-ethyl]-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl})-carbamic acid tert-butyl ester

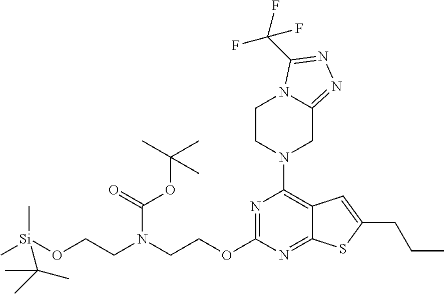

According to the same method to that described in Example 1-45, the title compound (1.3 g, 43%) was obtained by using the compound (1.43 g, 4.5 mmol) obtained from Preparation Example 9-98-1 and the compound (2 g, 4.96 mmol) obtained from Preparation Example 1-1-3.

¹H NMR (400 MHz, CDCl₃); δ 6.9 (1H, s), 5.27 (2H, s), 4.45 (2H, m), 4.32 (4H, m), 3.67 (4H, m), 3.38 (2H, m), 2.83 (2H, t), 1.72 (2H, m), 1.41 (9H, s), 1.02 (3H, t), 0.96 (9H, s), 0 (6H, s)

### Preparation Example 9-98-3

### Hydrochloric Acid Salt of

### 2-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethylamino}-ethanol

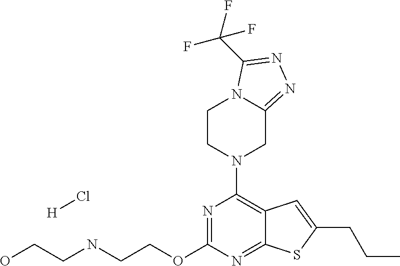

The title compound (900 mg, 90%) was obtained by treating the compound (1.3 g, 7.0 mmol) obtained from Preparation Example 9-98-2 according to the same method to that described in Example 1-4.

MS (M+1): 508.97

### Example 9-98

### (2-Hydroxy-ethyl)-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-carbamic acid methyl ester

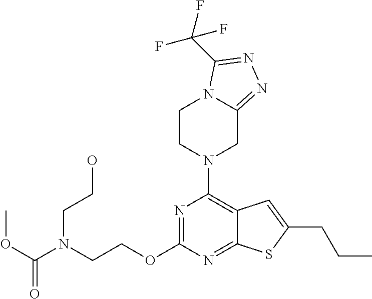

According to the same method to that described in Example 9-86, the title compound (20 mg, 20%) was obtained by using the compound (100 mg, 0.19 mmol) obtained from Preparation Example 9-98-3 and methyl chloroformate (0.015 mL, 0.19 mmol).

¹H NMR (400 MHz, CDCl₃); δ 6.95 (1H, s), 5.30 (2H, s), 4.54 (2H, m), 4.32 (4H, m), 3.80 (2H, m), 3.70 (3H, s), 3.56 (2H, m), 2.83 (2H, t), 1.76 (2H, m), 1.02 (3H, t)

### Preparation Example 9-99-1

### 3-(2-Hydroxy-ethyl)-oxazolidin-2-one

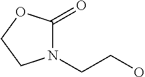

Di-ethanol amine (6 g) was diluted in tetrahydrofuran and stirred with sodium hydride and t-butyldimethylsilylchloride at 0° C. The title compound (2.0 g, 90%) was obtained by the treatment according to the same method to that described in Example 1-4

MS (M+1): 132.13

### Example 9-99

### 3-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-oxazolidin-2-one

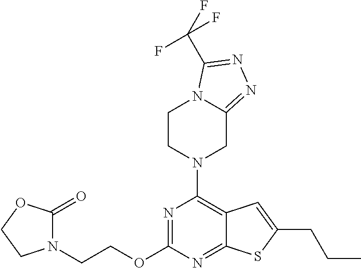

According to the same method to that described in Example 1-45, the title compound (130 mg, 40%) was obtained by using the compound (179 mg, 1.37 mmol) obtained from Preparation Example 9-99-1 and the compound (0.5 g, 1.24 mmol) obtained from Preparation Example 1-1-3.

¹H NMR (400 MHz, CDCl₃); δ 6.95 (1H, s), 5.31 (2H, s), 4.56 (2H, t), 4.33 (6H, m), 3.78 (2H, t), 3.69 (2H, t), 2.88 (2H, t), 1.76 (2H, m), 1.02 (3H, t)

### Example 9-100

### Acetic Acid

### 2-(acetyl-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-amino)-ethyl ester

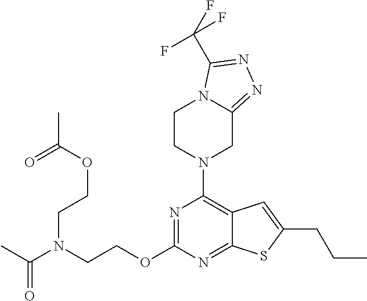

According to the same method to that described in Example 1-105, the title compound (20 mg, 18%) was obtained by using the compound (100 mg, 0.20 mmol) obtained from Preparation Example 9-98-3 and acetic anhydride (0.018 mL, 0.20 mmol).

¹H NMR (400 MHz, CDCl₃); δ 6.97 (1H, s), 5.31 (2H, s), 4.54 (2H, m), 4.36 (4H, m), 4.24 (2H, t), 3.78 (2H, m), 3.69 (2H, m), 2.20-2.14 (3H, d), 1.76 (2H, m), 1.02 (3H, t)

### Example 9-101

### N-(2-Hydroxy-ethyl)-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-formamide

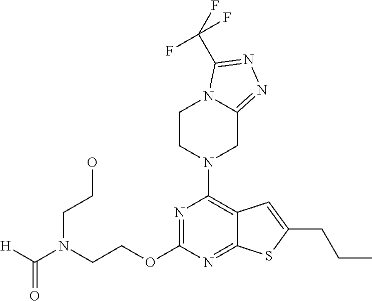

According to the same method to that described in Example 1-86, the title compound (5 mg, 5%) was obtained by using the compound (100 mg, 0.20 mmol) obtained from Preparation Example 9-98-3 and formic acid (0.008 mL, 0.22 mmol).

¹H NMR (400 MHz, CDCl₃); δ 8.15 (1H, d), 6.96 (1H, s), 5.30 (2H, s), 4.58 (2H, m), 4.32 (4H, m), 3.83 (2H, t), 3.75 (2H, m), 2.88 (2H, t), 1.75 (2H, m), 1.02 (3H, t)

### Example 9-102

### N-(2-Hydroxy-ethyl)-N-{2-[6-propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-acetamide

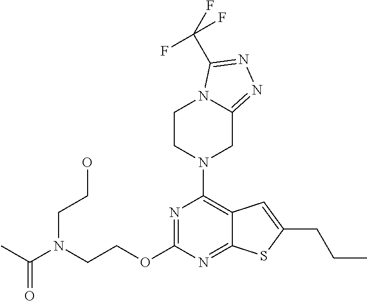

According to the same method to that described in Example 1-105, the title compound (5 mg, 5%) was obtained by using the compound (100 mg, 0.20 mmol) obtained from Preparation Example 9-98-3 and acetic anhydride (0.018 mL, 0.20 mmol).

¹H NMR (400 MHz, CDCl₃); δ 6.97 (1H, d), 5.31 (2H, s), 4.60 (1H, t), 4.54 (1H, t), 4.37 (2H, d), 4.32 (2H, d), 3.80 (4H, m), 3.71 (1H, t), 3.63 (1H, t), 2.86 (2H, m) 2.16 (3H, d), 1.75 (2H, m), 1.02 (3H, t)

### Example 9-103

### 3-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-propionitrile

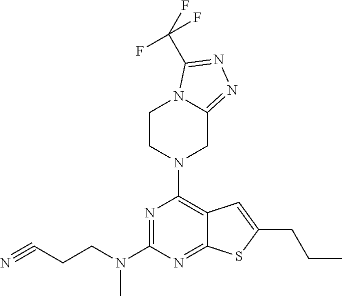

According to the same method to that described in Example 1-1, the title compound (300 mg, 27%) was obtained by using the compound (1.0 g, 2.48 mmol) obtained from Preparation Example 1-1-3 and N-methyl-β ?alaninenitrile (0.46 mL, 4.96 mmol).

¹H NMR (400 MHz, CDCl₃); δ 6.80 (1H, s), 5.18 (2H, s), 4.34 (2H, m), 4.22 (2H, m), 3.92 (2H, t), 3.27 (3H, s), 2.79 (2H, t), 2.70 (2H, t), 1.72 (2H, m), 1.00 (3H, t)

### Preparation Example 9-104-1

### 3-Methylamino-propionamide

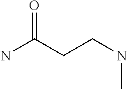

Potassium hydroxide (6.0 g, 107.14 mmol) was dissolved in t-butanol 15 mL. N-methyl-β-alaninenitrile was slowly added dropwise thereto and the reaction was carried out for 1 hour at 90° C. After the solid material was removed from the reaction solution, the reaction solution was cooled to −15° C. and water 5 mL was added thereto. The title compound (3.03 g, 81.6%) was obtained by distillation of the reaction solution under reduced pressure.

¹H NMR (400 MHz, CDCl₃); δ 7.53 (1H, br), 5.52 (1H, br), 2.84 (2H, t), 2.44 (3H, s), 2.37 (2H, t)

### Example 9-104

### 3-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-propionamide

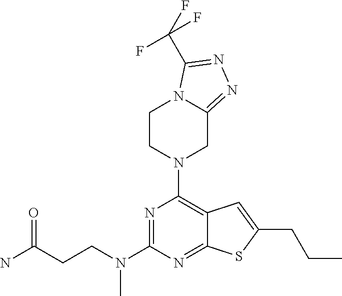

According to the same method to that described in Example 1-1, the title compound (396 mg, 68.1%) was obtained by using the compound (0.5 g, 1.24 mmol) obtained from Preparation Example 1-1-3 and the compound (253 mg, 2.48 mmol) obtained from Preparation Example 9-104-1.

¹H NMR (400 MHz, CDCl₃); δ 6.79 (1H, s), 6.58 (1H, br), 5.30 (1H, br), 5.17 (2H, s), 4.25 (4H, m), 3.88 (2H, t), 3.17 (3H, s), 2.78 (2H, t), 2.50 (2H, t), 1.71 (2H, m), 1.00 (3H, t)

### Preparation Example 9-105-1

### 2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethanol

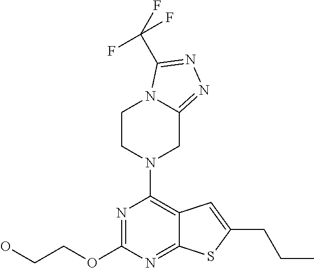

According to the same method to that described in Example 1-45, the title compound (1.30 g, 61.2%) was obtained by using ethylene glycol (0.55 mL, 9.92 mmol) and the compound (2.0 g, 4.96 mmol) obtained from Preparation Example 1-1-3.

¹H NMR (400 MHz, CDCl₃); δ 6.95 (1H, s), 5.31 (2H, s), 4.77 (1H, br), 4.51 (2H, t), 4.37 (2H, t), 4.30 (2H, t), 3.97 (2H, t), 2.80 (2H, t), 1.72 (2H, m), 1.00 (3H, t)

### Example 9-105

### 3-{2-[6-Propyl-4-(3-trifluoromethyl-5,6-dihydro-8H-[1,2,4]triazolo[4,3-a]pyrazin-7-yl)-thieno[2,3-d]pyrimidin-2-yloxy]-ethyl}-imidazolidine-2,4-dione

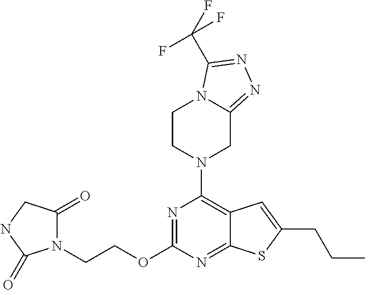

The compound (1.3 g, 3.03 mmol) obtained from Preparation Example 9-105-1 was dissolved in dichloromethane 50 mL. Triethylamine (0.84 mL, 6.06 mmol) was added thereto and cooled to 0° C. Methanesulfonylchloride (0.26 mL, 3.33 mmol) was slowly added to the reaction solution and the reaction was carried out for 30 minutes. Then, the reaction solution was washed with water and brine, dried with anhydrous magnesium sulfate and evaporated under reduced pressure. The remaining residue was dissolved in dimethylformamide 30 mL. Then, hydantoin (129 mg, 1.29 mmol) and potassium carbonate (150.65 mg, 1.09 mmol) were added thereto and the reaction was carried out for 16 hours at 70° C. The reaction solution was distilled under reduced pressure, diluted with ethyl acetate and washed with water and sodium hydrogen carbonate aqueous solution. After drying with magnesium sulfate and distillation under reduced pressure, the title compound (208.1 mg, 41.2%) was obtained by column-chromatography using 9:1 mixture of dichloromethane and methanol.

¹H NMR (400 MHz, DMSO); δ 8.01 (1H, s), 7.31 (1H, s), 5.15 (2H, s), 4.33 (2H, m), 4.24 (2H, m), 4.06 (2H, m), 3.75 (4H, s), 2.79 (2H, t), 1.63 (2H, m), 0.94 (3H, t)

### Example 9-106

### 2-{Methyl-[6-propyl-4-(3-trifluoromethyl-5,6-dihydro-8H-[1,2,4]triazolo[4,3-a]pyrazin-7-yl)-thieno[2,3-d]pyrimidin-2-yl]-amino}-ethanol

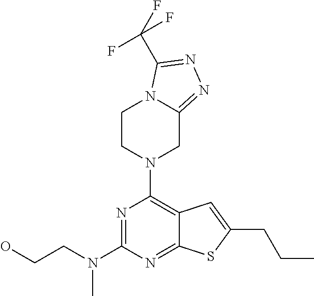

According to the same method to that described in Example 1-1, the title compound (3.12 g, 94.8%) was obtained by using the compound (3.0 g, 7.45 mmol) obtained from Preparation Example 1-1-3 and 2-(methylamino)ethanol (1.2 mL, 14.90 mmol).

¹H NMR (400 MHz, CDCl₃); δ 3.77 (1H, s), 5.18 (2H, s), 4.34 (2H, m), 4.21 (2H, m), 3.86 (2H, m), 3.79 (2H, m), 3.22 (3H, s), 2.77 (2H, t), 1.71 (2H, m), 0.98 (3H, t)

### Example 9-107

### [6-Propyl-4-(3-trifluoromethyl-5,6-dihydro-8H-[1,2,4]triazolo[4,3-a]pyrazin-7-yl)-thieno[2,3-d]pyrimidin-2-yl]-carbamic acid methyl ester

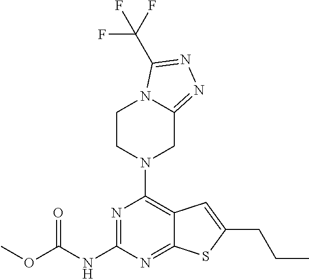

The compound (1 g, 2.61 mmol) obtained from Example 9-38 and triethylamine (0.79 g, 7.83 mmol) were dissolved in dichloromethane 20 mL. Phosgene (0.52 g, 5.21 mmol) (20% toluene solution) was slowly added dropwise thereto and stirred for 1 hour at room temperature. After the solvent was removed under reduced pressure, the remaining residue was dissolved in methanol 10 mL. Sodium methoxide (0.46 g, 2.61 mmol) was added thereto and stirred for 3 hours with reflux. After the reaction was completed, the solvent was removed under reduced pressure. The title compound (0.13 g, 0.29 mmol, yield: 11%) of yellow solid form was obtained by column-chromatography (developing solution: 5% methanol/methylene dichloride).

¹H NMR (400 MHz, CDCl₃); δ 7.37 (1H, s), 6.97 (1H, s), 5.32 (2H, s), 4.43 (2H, m), 4.38 (2H, m), 3.81 (3H, s), 2.85 (2H, t), 1.75 (2H, m), 1.01 (3H, t)

### Experiment 1

### Inhibition of Platelet Aggregation

Platelet Preparation for Aggregation Studies

Human platelet aggregation reaction test is a functional experiment method to verify whether a test compound is an agonist or an antagonist to ADP receptor. The test compound efficiently inhibited platelet aggregation reaction caused by ADP which is a well-known agonist. The platelet product isolated from whole blood was purchased from Chung-Nam blood center (Dae-joen, South Korea). The platelet was isolated from 400 mL of whole blood containing 56 mL of CPDA-1 anticoagulant. To isolate the platelet product, 400 mL of whole blood was centrifuged at 260×g for 5 minutes, and then obtained PRP (platelet rich plasma) was secondary centrifuged at 370×g for 6 minutes. Platelet pellet (10⁹/mL) was suspended with 45 mL of plasma and then agitated over 1 hour at 22° C. blood reservoir to stabilize it. The stabilized platelet product was used in the experiment within 24 hours. PCs (Platelet Concentrates) were gently transferred to a new 50 ml-falcon tube. The number of platelet was counted by using animal blood counter (ABC Vet, ABX diagnostics, France). The volume of 30 mL of PCs collected from same donor was centrifuged at 1500 g for 10 min at room temperature to prepare platelet-poor plasma (PPP). The platelet count was adjusted to platelet number of 3˜4×10⁸/mL by diluting with PPP.

Evaluation on Inhibitory Effect of ADP-Induced Aggregation

Inhibition of ADP-induced aggregation was determined in 96-well flat-bottom plates (Costar, USA) at room temperature. The diluted platelet was aliquoted into each well of 96 well plate in the volume of 188 μl. The volume of 2 μl of test compound dissolved in DMSO solution was added into each well to make the final concentration of 0.01, 0.03, 0.1, 0.3, 1, 3, 10, 30 μM. The platelet including test compounds was immediately mixed on a shaker at 1050 rpm for 1 min and incubated at room temperature for 20 min. Then, 10 μL volume of 400 μM ADP was added: the final concentration of 20 μM ADP in the total reaction volume of 0.2 mL/well. After shaking of the plate at room temperature for 3 min, the absorption of the samples was determined at 595 nm using a microtiter plate reader (Softmax, Molecular Devices).

Calculation of Ki

A dose-dependent curve on serial dilutions of ADP was accomplished by adding vehicle (DMSO) instead of antagonists in order to calculate EC₅₀ of ADP. Inhibition of aggregation was calculated from the increase of absorption at 595 nm at 3 min of the reaction incubation, compared to results of absorption between the 0 and 20 μM ADP control without antagonists. Sigmoidal dose-response curves and Ki were derived by non-linear regression analysis using the Prism software (GraphPad, San Diego Calif.). Some of measured efficacies (Mimic-human PRP, Ki) are shown in the following Table 1.

TABLE 1

| | Mimic-human PRP |
|---|---|
| Example No. | (Ki, μM) |
| 1-1 | 0.13 |
| 1-2 | NT |
| 1-3 | 0.164 |
| 1-4 | 0.3 |
| 1-5 | 0.21 |
| 1-5 | 0.16 |
| 1-7 | 0.77 |
| 1-8 | 0.3 |
| 1-9 | 0.5 |
| 1-10 | 0.178 |
| 1-11 | 1.378 |
| 1-12 | 0.226 |
| 1-13 | 0.09 |
| 1-14 | 0.034 |
| 1-15 | 0.095 |
| 1-16 | 0.045 |
| 1-17 | 0.08 |
| 1-18 | 0.034 |
| 1-19 | 0.056 |
| 1-20 | 0.19 |
| 1-21 | 0.22 |
| 1-22 | 0.13 |
| 1-23 | 0.094 |
| 1-24 | 0.088 |
| 1-25 | 0.1 |
| 1-26 | 1.2 |
| 1-27 | 0.072 |
| 1-28 | 0.176 |
| 1-29 | 0.06 |
| 1-30 | 0.09 |
| 1-31 | 7.6 |
| 1-32 | >4 |
| 1-33 | 0.25 |
| 1-34 | 0.081 |
| 1-35 | 0.054 |
| 1-36 | 1.2 |
| 1-37 | 0.85 |
| 1-38 | 0.18 |
| 1-39 | 0.55 |
| 1-40 | 0.175 |
| 1-41 | 1.6 |
| 1-42 | 7.3 |
| 1-43 | 0.67 |
| 1-44 | 0.26 |
| 1-45 | 1 |
| 1-46 | 0.058 |
| 1-47 | 1.9 |
| 1-48 | 1.3 |
| 1-49 | 0.23 |
| 1-50 | 0.04 |
| 1-51 | 0.8 |
| 1-52 | >4 |
| 1-53 | NT |
| 1-54 | 0.13 |
| 1-55 | 0.027 |
| 1-56 | NT |
| 1-57 | 0.054 |
| 1-58 | 0.7 |
| 1-59 | 1.45 |
| 1-60 | NT |
| 1-61 | 7.3 |
| 1-62 | 0.2 |
| 1-63 | 1 |
| 1-64 | >4 |
| 1-65 | 0.26 |
| 1-66 | 1.4 |
| 1-67 | >4 |
| 1-68 | 0.43 |
| 1-69 | 0.3 |
| 1-70 | 0.11 |
| 1-71 | >4 |
| 1-72 | >4 |
| 1-73 | >4 |
| 1-74 | 0.04 |
| 1-75 | 0.007 |
| 1-76 | NT |
| 1-77 | 0.06 |
| 1-78 | 0.2 |
| 1-79 | 1.25 |
| 1-80 | NT |
| 1-81 | 2.3 |
| 1-82 | >4 |
| 1-83 | 0.007 |
| 1-84 | 0.073 |
| 1-85 | 0.014 |
| 1-86 | 0.011 |
| 1-87 | 0.011 |
| 1-88 | 0.016 |
| 1-89 | 0.012 |
| 1-90 | 0.05 |
| 1-91 | 0.14 |
| 1-92 | 0.03 |
| 1-93 | 0.204 |
| 1-94 | 0.029 |
| 1-95 | 0.011 |
| 1-96 | 0.021 |
| 1-97 | 0.013 |
| 1-98 | 0.032 |
| 1-99 | 0.043 |
| 1-100 | 0.006 |
| 1-101 | 0.0035 |
| 1-102 | 0.0086 |
| 1-103 | 0.0066 |
| 1-104 | 0.086 |
| 1-105 | 0.0044 |
| 1-106 | 0.016 |
| 1-107 | 0.019 |
| 1-108 | 0.0093 |
| 1-109 | 0.026 |
| 1-110 | 0.06 |
| 1-111 | 0.59 |
| 1-112 | 0.26 |
| 1-113 | 0.036 |
| 1-114 | 0.0038 |
| 1-115 | 0.016 |
| 1-116 | 0.028 |
| 1-117 | 0.042 |
| 1-118 | 0.034 |
| 1-119 | 0.0072 |
| 1-120 | 0.016 |
| 1-121 | 0.019 |
| 1-122 | 0.467 |
| 1-123 | 0.367 |
| 1-124 | 0.0039 |
| 1-125 | 0.045 |
| 1-126 | 0.029 |
| 1-127 | 0.9 |
| 1-128 | 0.061 |
| 1-129 | NT |
| 1-130 | 0.132 |
| 1-131 | NT |
| 1-132 | 0.1 |
| 1-133 | 0.145 |
| 1-134 | 0.869 |
| 1-135 | 0.027 |
| 2-1 | NT |
| 2-2 | 0.832 |
| 2-3 | NT |
| 2-4 | NT |
| 2-5 | 0.857 |
| 2-6 | 0.715 |
| 2-7 | 0.669 |
| 2-8 | NT |
| 2-9 | NT |
| 2-10 | 0.773 |
| 2-11 | NT |
| 2-12 | 2.9 |
| 2-13 | NT |
| 2-14 | 0.329 |
| 2-15 | 0.223 |
| 2-16 | 0.817 |
| 2-17 | 0.44 |
| 2-18 | 0.31 |
| 2-19 | 0.55 |
| 2-20 | 0.04 |
| 3-1 | 0.031 |
| 3-2 | 0.017 |
| 3-3 | 0.03 |
| 3-4 | 0.23 |
| 3-5 | 0.026 |
| 3-6 | 0.14 |
| 3-7 | 0.3 |
| 3-8 | 0.61 |
| 3-9 | 3.7 |
| 3-10 | 0.064 |
| 3-11 | 0.07 |
| 3-12 | 0.05 |
| 3-13 | 0.43 |
| 3-14 | 1.1 |
| 3-15 | 0.3 |
| 3-16 | 27.6 |
| 3-17 | 3.2 |
| 3-18 | 4.3 |
| 3-19 | >4 |
| 3-20 | 0.84 |
| 3-21 | 0.39 |
| 3-22 | NT |
| 3-23 | >4 |
| 3-24 | 1.48 |
| 3-25 | 0.93 |
| 3-26 | 0.025 |
| 3-27 | 8.8 |
| 3-28 | 9.7 |
| 3-29 | 0.15 |
| 3-30 | >4 |
| 3-31 | 2 |
| 3-32 | >4 |
| 3-33 | >4 |
| 3-34 | >4 |
| 3-35 | 1.4 |
| 3-36 | 0.94 |
| 3-37 | 1.3 |
| 3-38 | 2.2 |
| 3-39 | 2.4 |
| 3-40 | 1.2 |
| 3-41 | 1.5 |
| 3-42 | 0.66 |
| 3-43 | >4 |
| 3-44 | 0.5 |
| 3-45 | >4 |
| 3-46 | >4 |
| 3-47 | >4 |
| 3-48 | >4 |
| 3-49 | NT |
| 3-50 | 0.141 |
| 3-51 | 1.17 |
| 3-52 | NT |
| 3-53 | 0.76 |
| 3-54 | 0.206 |
| 3-55 | 0.025 |
| 3-56 | 0.075 |
| 3-57 | 0.023 |
| 3-58 | 0.021 |
| 3-59 | 0.0015 |
| 3-60 | 0.0007 |
| 3-61 | 0.0054 |
| 3-62 | 0.0019 |
| 3-63 | 0.007 |
| 3-64 | 0.15 |
| 3-65 | 0.013 |
| 3-66 | 0.07 |
| 3-67 | 0.017 |
| 3-68 | 0.12 |
| 3-69 | 4.1 |
| 3-70 | 0.007 |
| 4-1 | NT |
| 4-2 | NT |
| 4-3 | 0.12 |
| 4-4 | NT |
| 4-5 | NT |
| 4-6 | 1.2 |
| 4-7 | 0.32 |
| 4-8 | 0.55 |
| 4-9 | 5.1 |
| 4-10 | 0.2 |
| 4-11 | 0.6 |
| 4-12 | 0.095 |
| 4-13 | 1.8 |
| 4-14 | 0.46 |
| 4-15 | 0.69 |
| 4-16 | >4 |
| 4-17 | 1.6 |
| 4-18 | 1.6 |
| 4-19 | 0.25 |
| 4-20 | NT |
| 4-21 | 0.14 |
| 4-22 | 1.6 |
| 4-23 | 6.85 |
| 4-24 | 8.1 |
| 4-25 | 1.9 |
| 4-26 | 2.1 |
| 4-27 | 1.5 |
| 4-28 | 0.67 |
| 4-29 | 0.48 |
| 4-30 | 0.83 |
| 4-31 | >4 |
| 4-32 | 0.15 |
| 4-33 | 0.1 |
| 4-34 | 0.013 |
| 4-35 | 0.03 |
| 4-36 | 0.3 |
| 4-37 | 0.25 |
| 4-38 | 0.25 |
| 4-39 | 0.16 |
| 4-40 | 0.27 |
| 4-41 | 0.7 |
| 4-42 | 0.16 |
| 4-43 | 2.7 |
| 4-44 | >4 |
| 4-45 | NT |
| 4-46 | 0.025 |
| 4-47 | 0.013 |
| 4-48 | 0.51 |
| 4-49 | 0.011 |
| 4-50 | 0.034 |
| 4-51 | 0.036 |
| 4-52 | 0.038 |
| 4-53 | 5.25 |
| 4-54 | 0.47 |
| 4-55 | 0.0042 |
| 4-56 | 0.0064 |
| 4-57 | 0.28 |
| 4-58 | 0.243 |
| 4-59 | 0.0084 |
| 5-1 | NT |
| 5-2 | NT |
| 5-3 | NT |
| 5-4 | 0.057 |
| 5-5 | 0.025 |
| 5-6 | >4 |
| 5-7 | 0.038 |
| 6-1 | NT |
| 6-2 | NT |
| 6-3 | 0.395 |
| 6-4 | 0.308 |
| 6-5 | 0.94 |
| 6-6 | 49%@40 μM* |
| 6-7 | 0.407 |
| 6-8 | 0.095 |
| 6-9 | 0.03 |
| 6-10 | NT |
| 6-11 | NT |
| 6-12 | 0.809 |
| 6-13 | 0.716 |
| 6-14 | 0.26 |
| 6-15 | 0.19 |
| 6-16 | 0.061 |
| 6-17 | 0.4 |
| 6-18 | 0.3 |
| 7-1 | 0.011 |
| 7-2 | 0.079 |
| 7-3 | 0.026 |
| 7-4 | 0.075 |
| 7-5 | 0.032 |
| 7-6 | 0.018 |
| 7-7 | 0.063 |
| 7-8 | 0.15 |
| 7-9 | 0.59 |
| 7-10 | 0.98 |
| 7-11 | 0.28 |
| 7-12 | 0.035 |
| 7-13 | 0.076 |
| 7-14 | 0.12 |
| 7-15 | 0.6 |
| 7-16 | 0.59 |
| 7-17 | 0.0028 |
| 7-18 | 0.0065 |
| 7-19 | 0.005 |
| 7-20 | 0.0048 |
| 7-21 | 0.021 |
| 7-22 | 0.004 |
| 7-23 | 0.012 |
| 7-24 | 0.015 |
| 7-25 | 0.018 |
| 7-26 | 0.0021 |
| 7-27 | 0.017 |
| 7-28 | 0.042 |
| 7-29 | 0.0031 |
| 7-30 | 0.0072 |
| 7-31 | 0.012 |
| 7-32 | 0.0008 |
| 7-33 | 0.0067 |
| 7-34 | 0.016 |
| 7-35 | 0.01 |
| 7-36 | 0.1 |
| 7-37 | 0.28 |
| 7-38 | 0.05 |
| 8-1 | 0.035 |
| 8-2 | 0.11 |
| 8-3 | 0.33 |
| 8-4 | 0.014 |
| 8-5 | NT |
| 8-6 | 0.12 |
| 8-7 | 0.032 |
| 8-8 | 0.085 |
| 8-9 | 0.15 |
| 8-10 | 0.11 |
| 8-11 | NT |
| 8-12 | NT |
| 8-13 | 0.003 |
| 8-14 | 0.003 |
| 8-15 | 0.007 |
| 8-16 | 0.035 |
| 8-17 | 0.035 |
| 8-18 | 0.041 |
| 8-19 | 0.15 |
| 8-20 | 0.41 |
| 8-21 | 0.27 |
| 8-22 | 0.22 |
| 8-23 | 0.57 |
| 8-24 | 1.4 |
| 8-25 | NT |
| 8-26 | NT |
| 8-27 | 0.26 |
| 8-28 | 0.045 |
| 8-29 | 0.17 |
| 8-30 | 0.14 |
| 8-31 | 0.019 |
| 8-32 | 0.062 |
| 8-33 | 0.043 |
| 8-34 | 0.045 |
| 8-35 | 0.049 |
| 8-36 | 0.045 |
| 8-37 | 0.043 |
| 8-38 | 0.11 |
| 8-39 | 0.076 |
| 9-1 | 0.018 |
| 9-2 | 0.008 |
| 9-3 | NT |
| 9-4 | 0.28 |
| 9-5 | 0.13 |
| 9-6 | NT |
| 9-7 | NT |
| 9-8 | NT |
| 9-9 | 0.3 |
| 9-10 | NT |
| 9-11 | 0.048 |
| 9-12 | NT |
| 9-13 | 0.3 |
| 9-14 | NT |
| 9-15 | 0.042 |
| 9-16 | 0.88 |
| 9-17 | 0.019 |
| 9-18 | 0.010 |
| 9-19 | 0.003 |
| 9-20 | 0.006 |
| 9-21 | 0.006 |
| 9-22 | 0.015 |
| 9-23 | 0.003 |
| 9-24 | 0.31 |
| 9-25 | 0.0057 |
| 9-26 | 0.016 |
| 9-27 | 0.0065 |
| 9-28 | 0.00016 |
| 9-29 | 0.0022 |
| 9-30 | 0.0003 |
| 9-31 | 0.087 |
| 9-32 | 0.029 |
| 9-33 | 0.041 |
| 9-34 | 0.015 |
| 9-35 | 0.22 |
| 9-36 | 0.17 |
| 9-37 | 0.20 |
| 9-38 | 0.049 |
| 9-39 | NT |
| 9-40 | 0.022 |
| 9-41 | 0.037 |
| 9-42 | 0.027 |
| 9-43 | 0.33 |
| 9-44 | 0.0098 |
| 9-45 | 0.038 |
| 9-46 | 0.53 |
| 9-47 | 3.9 |
| 9-48 | 0.051 |
| 9-49 | 0.05 |
| 9-50 | 0.23 |
| 9-51 | 0.19 |
| 9-52 | 0.016 |
| 9-53 | 0.031 |
| 9-54 | 0.13 |
| 9-55 | 0.34 |
| 9-56 | 0.03 |
| 9-57 | 0.41 |
| 9-58 | 0.022 |
| 9-59 | >10 |
| 9-60 | 1.2 |
| 9-61 | >10 |
| 9-62 | 0.4 |
| 9-63 | 2 |
| 9-64 | 1.4 |
| 9-65 | 9.3 |
| 9-66 | 0.0052 |
| 9-67 | 0.097 |
| 9-68 | 0.26 |
| 9-69 | 0.55 |
| 9-70 | 0.006 |
| 9-71 | 0.0007 |
| 9-72 | 0.0006 |
| 9-73 | 0.016 |
| 9-74 | 0.0038 |
| 9-75 | 0.003 |
| 9-76 | 0.022 |
| 9-77 | 0.006 |
| 9-78 | 0.025 |
| 9-79 | 0.0034 |
| 9-80 | 0.015 |
| 9-81 | 0.007 |
| 9-82 | 0.005 |
| 9-83 | 0.0003 |
| 9-84 | 0.68 |
| 9-85 | 2 |
| 9-86 | 0.69 |
| 9-87 | 0.43 |
| 9-88 | 0.42 |
| 9-89 | 0.29 |
| 9-90 | 0.11 |
| 9-91 | 0.19 |
| 9-92 | 1.3 |
| 9-93 | 0.052 |
| 9-94 | 0.28 |
| 9-95 | 0.0072 |
| 9-96 | 0.078 |
| 9-97 | 0.006 |
| 9-98 | 0.023 |
| 9-99 | 0.004 |
| 9-100 | 0.038 |
| 9-101 | 0.012 |
| 9-102 | 0.008 |
| 9-103 | 0.07 |
| 9-104 | 0.014 |
| 9-105 | >3 |
| 9-106 | 0.14 |
| 9-107 | 0.17 |

NT: Not Tested
*“A %@B μM” means A % inhibition at B μM concentration.

### Experiment 2

### Human P2Y12 Receptor Cell Membrane Binding Assay with [³H] 2-Mes-ADP

Whether test compounds bind to P2Y12 receptor is measured by [3H]-2MesADP binding in HEK cells expressing P2Y12 receptor. The efficacy according to the amount of test compound can be known through the above test, competitive binding test to P2Y12 receptor, in which the binding material (2-Mes-ADP) is labeled with radioactivity isotope and then the extent of inhibition against the binding of radioactively labeled material is measured.

Preparation of P2Y12 Membrane Protein Fraction

Harvested P2Y12 cells were resuspended with Hypotonic buffer (10 mM HEPES, 10 mM NaCl, 1 mM EDTA, 1 mM EGTA pH 7.4) containing protease inhibitor cocktail. Following the incubation on ice for 15 min, cells were homogenized on ice (Glass/Teflon homogenizer; 1,000 rpm, 10 strokes). The supernant was taken from centrifugation at 500×g for 15 min and then recentrifuged the supernant at 30,000 rpm for 30 min. The membrane pellet was adjusted to 1 mg/mL concentration and stored at −80° C.

P2Y12 Receptor Cell Membrane Binding Assay with [3H] 2-Mes-ADP

The volume of 100 μL of 200 μg membrane protein was aliquoted into 96 well plate. 50 μL volume of test compounds and [³H] 2-Mes-ADP (final: 1 nM) were added into each well. The total reaction of 0.2 ml/well was incubated for 1 hour at R.T. The reaction was terminated by filtration through GF/B filter using a 96-well cell harvester. The filter was washed ten times with ice cold wash buffer (10 mM HEPES, pH 7.4, 138 mM NaCl). Radioactivity bound to P2Y12 receptor membrane was counted in a scintillation counter. Total binding was determined with CPM of test group having no compound and non-specific binding was determined with CPM of test group containing 10 μM 2-Mes-ADP. Binding activities (μM) of each tested compound are shown in the following Table 2. Binding inhibition percent was calculated as follows: [100−(specific binding in the presence of test material/specific binding in the absence of test material×100)].

TABLE 2

| | Binding activity |
|---|---|
| Example No. | (IC50, μM) |
| 1-1 | 0.02 |
| 1-2 | 0.16 |
| 1-3 | 0.092 |
| 1-4 | 0.064 |
| 1-5 | 0.096 |
| 1-5 | 0.1 |
| 1-7 | 0.22 |
| 1-8 | 0.056 |
| 1-9 | 0.07 |
| 1-10 | 0.11 |
| 1-11 | 0.26 |
| 1-12 | 0.084 |
| 1-13 | 0.083 |
| 1-14 | 0.06 |
| 1-15 | 0.062 |
| 1-16 | 0.036 |
| 1-17 | 0.041 |
| 1-18 | 0.054 |
| 1-19 | 0.055 |
| 1-20 | 0.163 |
| 1-21 | 0.452 |
| 1-22 | 0.065 |
| 1-23 | 0.09 |
| 1-24 | 0.054 |
| 1-25 | 0.032 |
| 1-26 | 0.06 |
| 1-27 | 0.056 |
| 1-28 | 0.04 |
| 1-29 | 0.05 |
| 1-30 | 0.066 |
| 1-31 | 0.11 |
| 1-32 | 0.095 |
| 1-33 | 0.78 |
| 1-34 | 0.143 |
| 1-35 | 0.135 |
| 1-36 | 0.17 |
| 1-37 | 0.55 |
| 1-38 | 0.1 |
| 1-39 | 0.14 |
| 1-40 | 0.2 |
| 1-41 | 0.536 |
| 1-42 | 1.17 |
| 1-43 | 0.134 |
| 1-44 | 0.059 |
| 1-45 | 0.34 |
| 1-46 | 0.021 |
| 1-47 | 0.28 |
| 1-48 | 0.4 |
| 1-49 | 0.098 |
| 1-50 | 0.11 |
| 1-51 | 0.22 |
| 1-52 | 1.03 |
| 1-53 | NT |
| 1-54 | 0.143 |
| 1-55 | 0.06 |
| 1-56 | NT |
| 1-57 | 0.015 |
| 1-58 | 0.19 |
| 1-59 | 0.44 |
| 1-60 | NT |
| 1-61 | 0.011 |
| 1-62 | 0.32 |
| 1-63 | 0.52 |
| 1-64 | 0.17 |
| 1-65 | 0.257 |
| 1-66 | 0.2 |
| 1-67 | 0.643 |
| 1-68 | 0.1 |
| 1-69 | 0.082 |
| 1-70 | 0.167 |
| 1-71 | 0.526 |
| 1-72 | 0.517 |
| 1-73 | 0.36 |
| 1-74 | 0.033 |
| 1-75 | 0.032 |
| 1-76 | 0.028 |
| 1-77 | 0.047 |
| 1-78 | 0.11 |
| 1-79 | 0.37 |
| 1-80 | 0.043 |
| 1-81 | 0.32 |
| 1-82 | 14.3 |
| 1-83 | 0.32 |
| 1-84 | 0.16 |
| 1-85 | 0.096 |
| 1-86 | 0.024 |
| 1-87 | 0.067 |
| 1-88 | NT |
| 1-89 | 0.055 |
| 1-90 | 0.11 |
| 1-91 | 0.19 |
| 1-92 | NT |
| 1-93 | NT |
| 1-94 | NT |
| 1-95 | NT |
| 1-96 | 0.63 |
| 1-97 | 0.14 |
| 1-98 | 0.47 |
| 1-99 | 0.13 |
| 1-100 | NT |
| 1-101 | NT |
| 1-102 | NT |
| 1-103 | 0.05 |
| 1-104 | 0.08 |
| 1-105 | NT |
| 1-106 | NT |
| 1-107 | NT |
| 1-108 | NT |
| 1-109 | 0.08 |
| 1-110 | 0.67 |
| 1-111 | 0.15 |
| 1-112 | 0.05 |
| 1-113 | 0.032 |
| 1-114 | NT |
| 1-115 | 0.11 |
| 1-116 | 0.07 |
| 1-117 | 0.22 |
| 1-118 | NT |
| 1-119 | NT |
| 1-120 | 0.038 |
| 1-121 | NT |
| 1-122 | 0.22 |
| 1-123 | 0.26 |
| 1-124 | NT |
| 1-125 | 0.15 |
| 1-126 | 0.13 |
| 1-127 | 0.1 |
| 1-128 | 0.1 |
| 1-129 | NT |
| 1-130 | 0.32 |
| 1-131 | 1.93 |
| 1-132 | 0.047 |
| 1-133 | NT |
| 1-134 | 1 |
| 1-135 | 0.067 |
| 2-1 | 1.1 |
| 2-2 | 0.097 |
| 2-3 | 0.847 |
| 2-4 | 8.92 |
| 2-5 | 0.71 |
| 2-6 | 0.835 |
| 2-7 | 0.21 |
| 2-8 | 2.28 |
| 2-9 | 2.17 |
| 2-10 | 0.22 |
| 2-11 | 1.82 |
| 2-12 | 0.46 |
| 2-13 | 1.1 |
| 2-14 | 0.1 |
| 2-15 | 0.24 |
| 2-16 | 0.188 |
| 2-17 | 0.367 |
| 2-18 | 0.33 |
| 2-19 | 0.362 |
| 2-20 | 0.26 |
| 3-1 | 0.14 |
| 3-2 | 0.2 |
| 3-3 | 0.113 |
| 3-4 | 0.17 |
| 3-5 | 0.091 |
| 3-6 | 0.69 |
| 3-7 | 0.54 |
| 3-8 | 0.331 |
| 3-9 | 0.826 |
| 3-10 | 0.14 |
| 3-11 | 0.072 |
| 3-12 | 0.16 |
| 3-13 | 0.41 |
| 3-14 | 0.24 |
| 3-15 | 0.053 |
| 3-16 | 0.039 |
| 3-17 | 0.11 |
| 3-18 | 0.4 |
| 3-19 | 0.17 |
| 3-20 | 0.084 |
| 3-21 | 0.11 |
| 3-22 | 0.13 |
| 3-23 | 0.37 |
| 3-24 | 0.16 |
| 3-25 | 0.17 |
| 3-26 | 0.149 |
| 3-27 | 5.38 |
| 3-28 | 0.179 |
| 3-29 | 0.22 |
| 3-30 | 0.28 |
| 3-31 | 0.196 |
| 3-32 | 0.65 |
| 3-33 | 2.22 |
| 3-34 | 0.473 |
| 3-35 | 0.099 |
| 3-36 | 0.26 |
| 3-37 | 0.198 |
| 3-38 | 0.179 |
| 3-39 | 0.3 |
| 3-40 | 0.23 |
| 3-41 | 0.39 |
| 3-42 | 0.25 |
| 3-43 | 0.65 |
| 3-44 | 0.18 |
| 3-45 | 1.9 |
| 3-46 | 2.33 |
| 3-47 | 0.375 |
| 3-48 | 0.34 |
| 3-49 | 0.406 |
| 3-50 | 0.596 |
| 3-51 | 0.7 |
| 3-52 | 20%@30 μM* |
| 3-53 | 0.57 |
| 3-54 | NT |
| 3-55 | 0.15 |
| 3-56 | NT |
| 3-57 | NT |
| 3-58 | 0.4 |
| 3-59 | NT |
| 3-60 | NT |
| 3-61 | NT |
| 3-62 | NT |
| 3-63 | 0.043 |
| 3-64 | 0.11 |
| 3-65 | 0.18 |
| 3-66 | 0.15 |
| 3-67 | 0.09 |
| 3-68 | 0.14 |
| 3-69 | 0.18 |
| 3-70 | 0.017 |
| 4-1 | 0.24 |
| 4-2 | 3.05 |
| 4-3 | 0.63 |
| 4-4 | 1 |
| 4-5 | 1.58 |
| 4-6 | 2.89 |
| 4-7 | 0.62 |
| 4-8 | 0.47 |
| 4-9 | 1.1 |
| 4-10 | 1.55 |
| 4-11 | 0.66 |
| 4-12 | 1.1 |
| 4-13 | 4.92 |
| 4-14 | 1.29 |
| 4-15 | 0.63 |
| 4-16 | 0.65 |
| 4-17 | 0.21 |
| 4-18 | 1.53 |
| 4-19 | 0.5 |
| 4-20 | 4.21 |
| 4-21 | 1.11 |
| 4-22 | 0.85 |
| 4-23 | 0.42 |
| 4-24 | 0.7 |
| 4-25 | 0.353 |
| 4-26 | 0.16 |
| 4-27 | 0.13 |
| 4-28 | 0.33 |
| 4-29 | 0.33 |
| 4-30 | 6.5 |
| 4-31 | 0.42 |
| 4-32 | 0.23 |
| 4-33 | 0.075 |
| 4-34 | 0.068 |
| 4-35 | 0.13 |
| 4-36 | 0.25 |
| 4-37 | 0.192 |
| 4-38 | 0.32 |
| 4-39 | 0.1 |
| 4-40 | 0.23 |
| 4-41 | 0.41 |
| 4-42 | 0.029 |
| 4-43 | 0.147 |
| 4-44 | 1.6 |
| 4-45 | NT |
| 4-46 | NT |
| 4-47 | 0.024 |
| 4-48 | 0.21 |
| 4-49 | 0.033 |
| 4-50 | 0.057 |
| 4-51 | 0.04 |
| 4-52 | 0.014 |
| 4-53 | 0.3 |
| 4-54 | 0.37 |
| 4-55 | 0.174 |
| 4-56 | 0.17 |
| 4-57 | 0.24 |
| 4-58 | 0.3 |
| 4-59 | 0.04 |
| 5-1 | 0.68 |
| 5-2 | 2.32 |
| 5-3 | 1.15 |
| 5-4 | 0.072 |
| 5-5 | 0.17 |
| 5-6 | 0.63 |
| 5-7 | 0.032 |
| 6-1 | 0.133 |
| 6-2 | 1.2 |
| 6-3 | 0.62 |
| 6-4 | 0.128 |
| 6-5 | 0.266 |
| 6-6 | 0.315 |
| 6-7 | 0.49 |
| 6-8 | 0.13 |
| 6-9 | 0.12 |
| 6-10 | 7 |
| 6-11 | 39.3%@30 μM* |
| 6-12 | 0.76 |
| 6-13 | 2.46 |
| 6-14 | 0.322 |
| 6-15 | 0.4 |
| 6-16 | 0.31 |
| 6-17 | 0.75 |
| 6-18 | 0.67 |
| 6-19 | 1.95 |
| 6-20 | 7.64 |
| 6-21 | 14.5 |
| 6-22 | 10 |
| 6-23 | 12 |
| 6-24 | 30 |
| 6-25 | 38.6%@30 μM* |
| 6-26 | 16.4 |
| 6-27 | 10 |
| 6-28 | 5.6 |
| 6-29 | 29.8 |
| 6-30 | 1.04 |
| 6-31 | 4.66 |
| 6-32 | 4.64 |
| 6-33 | 2.37 |
| 6-34 | 0.12 |
| 6-35 | 2.3 |
| 6-36 | 0.64 |
| 6-37 | 0.4 |
| 6-38 | 4.42 |
| 6-39 | 1.5 |
| 6-40 | 1.95 |
| 6-41 | 23.6%@30 μM* |
| 6-42 | 26.4%@30 μM* |
| 6-43 | 46.4 |
| 6-44 | 21.5 |

NT: Not Tested
*“A %@B μM” means A % inhibition at B μM concentration.

1. A compound having the following formula 1 or pharmaceutically acceptable salt thereof: 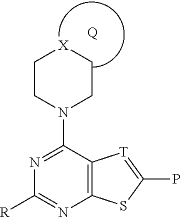
wherein
X represents N or C,
T represents N or C,
the ring Q represents a 3- to 7-membered aromatic ring which comprises 0 to 3 nitrogen atoms as ring members and is optionally benzo-fused, wherein the aromatic ring may be optionally substituted with oxo; alkyl; halogenoalkyl; hydroxyalkyl; alkoxy; aryl; or 3- to 7-membered heteroaryl comprising 1 to 3 heteroatoms selected from nitrogen atom, oxygen atom and sulfur atom,
P represents alkyl being optionally substituted with halogen, and
R represents a group selected from the following groups:
(i) -alkyl-R¹
wherein R¹ is selected from hydroxy; carboxy; carbamoyl; thiocarbamoyl; alkoxycarbonyl; aryloxy being optionally substituted with carboxy or alkoxycarbonyl; arylcarbonyloxy; 3- to 7-membered heteroaryl comprising 1 to 3 heteroatoms selected from nitrogen atom, oxygen atom and sulfur atom, and being optionally substituted with carboxy or alkoxycarbonyl; and 3- to 7-membered heterocycle comprising 1 to 3 heteroatoms selected from nitrogen atom, oxygen atom and sulfur atom, and being optionally substituted with hydroxy.
(ii) —NR²R³
wherein each of R² and R³ is independently selected from hydrogen; alkyl being optionally substituted with amino (said amino is optionally substituted with formyl, alkylcarbonyl, alkoxycarbonyl or carbamoyl), cyano, carbamoyl, hydroxy, carboxy, hydroxyaryl, alkoxy, alkoxycarbonyl, hydroxyalkoxy, 3- to 7-membered heterocycle comprising 1 to 3 heteroatoms selected from nitrogen atom, oxygen atom and sulfur atom (said heterocycle is optionally substituted with oxo, aralkyl, alkylcarbonyl or alkoxycarbonyl), or 3- to 7-membered heteroaryl comprising 1 to 3 heteroatoms selected from nitrogen atom, oxygen atom and sulfur atom; alkylcarbonyl; formyl; alkoxycarbonyl; carbamoyl; cycloalkyl being optionally substituted with hydroxy or hydroxyalkoxy; 3- to 7-membered heterocycle comprising 1 to 3 heteroatoms selected from nitrogen atom, oxygen atom and sulfur atom (said heterocycle is optionally substituted with alkylcarbonyl); aryl; aralkyl; 3- to 7-membered heteroaryl comprising 1 to 3 heteroatoms selected from nitrogen atom, oxygen atom and sulfur atom, and being optionally substituted with carboxy or alkoxycarbonyl.
(iii) —O—R⁴
wherein R⁴ is selected from the following groups:
(a) hydrogen,
(b) alkyl being optionally substituted with hydroxy; alkoxy; amino(said amino is optionally substituted with alkyl, hydroxyalkyl, alkylcarbonyloxyalkyl, formyl, alkylcarbonyl, carbamoyl, alkylaminocarbonyl or alkoxycarbonyl); halogen; cyano; carbamoyl; hydrazidocarbonyl; carboxy; oxo; alkylcarbonyloxyalkoxy; aryl being optionally substituted with halogen; 3- to 7-membered heteroaryl comprising 1 to 3 heteroatoms selected from nitrogen atom, oxygen atom and sulfur atom, and being optionally substituted with carboxyalkyl or alkoxycarbonylalkyl; 3- to 7-membered heterocycle comprising 1 to 3 heteroatoms selected from nitrogen atom, oxygen atom and sulfur atom, and being optionally substituted with oxo, alkylcarbonyl or alkoxycarbonyl; 3- to 7-membered heteroarylcarbonylamino comprising 1 to 3 heteroatoms selected from nitrogen atom, oxygen atom and sulfur atom in the heteroaryl, and being optionally substituted with halogen; 3- to 7-membered heterocyclylcarbonyl comprising 1 to 3 heteroatoms selected from nitrogen atom, oxygen atom and sulfur atom in the heterocycle; 3- to 7-membered heterocyclylcarbonylamino comprising 1 to 3 heteroatoms selected from nitrogen atom, oxygen atom and sulfur atom in the heterocycle; aryloxycarbonylamino being optionally substituted with halogen; cycloalkylaminocarbonyl; or arylcarbonylamino being optionally substituted with halogen,
(c) cycloalkyl being optionally benzo-fused,
(d) alkylaminoalkyl being optionally substituted with alkoxycarbonyl or carboxy,
(e) cycloalkylcarbonylaminoalkyl,
(f) cycloalkylsulfonylaminoalkyl,
(g) alkylcarbonylaminoalkyl being optionally substituted with hydroxy, halogen, amino, alkoxy, alkylsulfonyl or aminosulfonyl,
(h) alkylsulfonylaminoalkyl being optionally substituted with halogen,
(i) aryl being optionally substituted with cyano; formyl; carboxy; alkoxycarbonyl; hydroxyalkyl; carboxyalkyl; alkoxycarbonylalkyl; carboxyalkoxy; alkoxycarbonylalkoxy; or 3- to 7-membered heterocycle comprising 1 to 3 heteroatoms selected from nitrogen atom, oxygen atom and sulfur atom,
(j) 3- to 7-membered heterocycle comprising 1 to 3 heteroatoms selected from nitrogen atom, oxygen atom and sulfur atom, and being optionally substituted with alkyl or alkylcarbonyl,
(k) 3- to 7-membered heteroaryl comprising 1 to 3 heteroatoms selected from nitrogen atom, oxygen atom and sulfur atom.
(iv) —S—R⁵
wherein R⁵ is selected from aryl, aralkyl or 3- to 7-membered heteroaryl comprising 1 to 3 heteroatoms selected from nitrogen atom, oxygen atom and sulfur atom.
(v) —C(═O)—R⁶
wherein R⁶ is selected from hydroxy; alkoxy; amino; alkylamino being optionally substituted with cyano, hydroxy, carboxy, alkoxycarbonyl or aryl; arylamino; and 3- to 7-membered heterocycle comprising 1 to 3 heteroatoms selected from nitrogen atom, oxygen atom and sulfur atom, and being optionally substituted with hydroxy, carboxy, alkyl or alkoxycarbonyl.
(vi) 3- to 7-membered heteroaryl comprising 1 to 4 heteroatoms selected from nitrogen atom, oxygen atom and sulfur atom, said heteroaryl being optionally substituted with one or more substituents selected from alkyl; amino; alkoxy; alkoxycarbonyl; aryl; carboxy; and nitro where the substituent is unsubstituted or mono- or disubstituted with hydroxy, cyano, carboxy, alkoxy, formyl, alkylcarbonyl, alkoxycarbonyl, cycloalkyl, aryl or amino.
(vii) saturated or partially unsaturated, single or fused 3- to 10-membered heterocycle comprising 1 to 4 heteroatoms selected from nitrogen atom, oxygen atom and sulfur atom, said heterocycle being connected to the backbone through a ring member nitrogen and being optionally substituted with one or more substituents selected from the following groups:
(a) hydroxy, halogen, oxo, cyano, carboxy, hydroxyimino, hydrazidocarbonyl,
(b) amino being unsubstituted or independently mono- or disubstituted with alkyl (said alkyl is optionally substituted with hydroxy), formyl, alkylcarbonyl or alkoxycarbonyl,
(c) carbamoyl being unsubstituted or mono- or disubstituted with alkyl, cycloalkyl, hydroxy, hydroxyalkyl, aminoalkyl or aralkylsulfonyl,
(d) alkoxyimino being optionally substituted with aryl,
(e) alkyl being optionally substituted with hydroxy, halogen or amino (said amino is optionally substituted with alkylcarbonyl or alkoxycarbonyl),
(f) alkoxy,
(g) alkylcarbonyl being optionally substituted with hydroxy or halogen,
(h) alkoxycarbonyl being optionally substituted with alkylcarbonyloxy,
(i) alkylsulfonyl,
(j) alkylcarbonyloxy,
(k) alkylcarbonylamino being optionally substituted with hydroxy; amino; cyano; halogen; alkoxy; or 3- to 7-membered heteroaryl comprising 1 to 3 heteroatoms selected from nitrogen atom, oxygen atom and sulfur atom, and being optionally substituted with amino,
(l) cycloalkylcarbonylamino,
(m) 3- to 7-membered heteroarylcarbonylamino comprising 1 to 3 heteroatoms selected from nitrogen atom, oxygen atom and sulfur atom in the heteroaryl, and being optionally substituted with halogen,
(n) alkylsulfonylamino,
(o) aryl being optionally substituted with hydroxy,
(p) cycloalkyl,
(q) cycloalkylalkyl,
(r) aryloxycarbonylamino being optionally substituted with halogen,
(s) arylcarbonylamino being optionally substituted with halogen,
(t) cycloalkylaminocarbonylamino,
(u) arylaminocarbonylamino being optionally substituted with halogen,
(v) 3- to 7-membered heteroarylsulfonylaminocarbonylamino comprising 1 to 3 heteroatoms selected from nitrogen atom, oxygen atom and sulfur atom in the heteroaryl, and being optionally substituted with halogen, and
(w) 3- to 7-membered heterocyclylcarbonyl comprising 1 to 3 heteroatoms selected from nitrogen atom, oxygen atom and sulfur atom in the heterocycle;
(viii) azido.

2. The compound having the formula 1 according to claim 1, or pharmaceutically acceptable salt thereof, wherein P, Q, R, T and X are defined as follows:
X represents N or C,
T represents N or C,
the ring Q represents a 3- to 7-membered aromatic ring which comprises 0 to 3 nitrogen atoms as ring members and is optionally benzo-fused, wherein the aromatic ring may be optionally substituted with oxo; alkyl; halogenoalkyl; hydroxyalkyl; alkoxy; aryl; or 3- to 7-membered heteroaryl comprising 1 to 3 heteroatoms selected from nitrogen atom, oxygen atom and sulfur atom,
P represents C₁-C₆-alkyl being optionally substituted with halogen, and
R represents a group selected from the following groups:
(i) —C₁-C₆-alkyl-R¹
wherein R¹ is selected from hydroxy; carboxy; carbamoyl; thiocarbamoyl; C₁-C₆-alkoxycarbonyl; C₆-C₁₀-aryloxy being optionally substituted with carboxy or C₁-C₆-alkoxycarbonyl; C₆-C₁₀-arylcarbonyloxy; 5- to 6-membered heteroaryl comprising 1 to 2 heteroatoms selected from nitrogen atom and sulfur atom, and being optionally substituted with carboxy or C₁-C₆-alkoxycarbonyl; and 5- to 6-membered heterocycle comprising 1 to 2 nitrogen atoms, and being optionally substituted with hydroxy.
(ii) —NR²R³
wherein each of R² and R³ is independently selected from hydrogen; C₁-C₆-alkyl being optionally substituted with amino (said amino is optionally substituted with carbamoyl), hydroxy, carboxy, hydroxy-C₆-C₁₀-aryl, C₁-C₆-alkoxy, C₁-C₆-alkoxycarbonyl, hydroxyl-C₁-C₆-alkoxy, or 5- to 6-membered heterocycle comprising 1 to 2 heteroatoms selected from nitrogen atom and sulfur atom (said heterocycle is optionally substituted with oxo or C₆-C₁₀-aryl-C₁-C₆-alkyl); C₃-C₆-cycloalkyl being optionally substituted with hydroxy or hydroxy-C₁-C₆-alkoxy; 4- to 6-membered heterocycle comprising 1 to 2 nitrogen atoms; C₆-C₁₀-aryl; C₆-C₁₀-aryl-C₁-C₆-alkyl; 5- to 6-membered heteroaryl comprising 1 to 2 heteroatoms selected from nitrogen atom and sulfur atom, and being optionally substituted with carboxy or C₁-C₆-alkoxycarbonyl.
(iii) —O—R⁴
wherein R⁴ is selected from the following groups:
(a) hydrogen,
(b) C₁-C₆-alkyl being optionally substituted with hydroxy; C₁-C₆-alkoxy; amino (said amino is optionally substituted with formyl or C₁-C₆-alkylcarbonyl); oxo; C₁-C₆-alkylcarbonyloxy-C₁-C₆-alkoxy; C₆-C₁₀-aryl being optionally substituted with halogen; 5- to 6-membered heteroaryl comprising 1 to 2 heteroatoms selected from nitrogen atom and oxygen, and being optionally substituted with carboxy-C₁-C₆-alkyl or C₁-C₆-alkoxycarbonyl-C₁-C₆-alkyl; 4- to 6-membered heterocycle comprising 1 to 2 heteroatoms selected from nitrogen atom and oxygen atom, and being optionally substituted with oxo; or 5- to 6-membered heteroarylcarbonylamino comprising 1 to 2 nitrogen atoms in the heteroaryl,
(c) C₃-C₆-cycloalkyl being optionally benzo-fused,
(d) C₁-C₆-alkylamino-C₁-C₆-alkyl being optionally substituted with C₁-C₆-alkoxycarbonyl or carboxy,
(e) C₃-C₆-cycloalkylcarbonylamino-C₁-C₆-alkyl,
(f) C₃-C₆-cycloalkylsulfonylamino-C₁-C₆-alkyl,
(g) C₁-C₆-alkylcarbonylamino-C₁-C₆-alkyl being optionally substituted with hydroxy, halogen, amino, C₁-C₆-alkoxy, C₁-C₆-alkylsulfonyl or aminosulfonyl,
(h) C₁-C₆-alkylsulfonylamino-C₁-C₆-alkyl being optionally substituted with halogen,
(i) C₆-C₁₀-aryl being optionally substituted with cyano; formyl; carboxy; C₁-C₆-alkoxycarbonyl; hydroxyl-C₁-C₆-alkyl; carboxy-C₁-C₆-alkyl; C₁-C₆-alkoxycarbonyl-C₁-C₆-alkyl; carboxy-C₁-C₆-alkoxy; C₁-C₆-alkoxycarbonyl-C₁-C₆-alkoxy; or 5- to 6-membered heterocycle comprising 1 to 2 nitrogen atoms,
(j) 4- to 6-membered heterocycle comprising 1 to 2 heteroatoms selected from nitrogen atom and oxygen atom, and being optionally substituted with C₁-C₆-alkyl,
(k) 5- to 6-membered heteroaryl comprising 1 to 2 nitrogen atoms.
(iv) —S—R⁵
wherein R⁵ is selected from C₆-C₁₀-aryl, C₆-C₁₀-aryl-C₁-C₆-alkyl or 5- to 6-membered heteroaryl comprising 1 to 2 nitrogen atoms.
(v) —C(═O)—R⁶
wherein R⁶ is selected from hydroxy; C₁-C₆-alkoxy; amino; C₁-C₆-alkylamino being optionally substituted with cyano, hydroxy, carboxy, C₁-C₆-alkoxycarbonyl or C₆-C₁₀-aryl; C₆-C₁₀-arylamino; and 5- to 6-membered heterocycle comprising 1 to 2 nitrogen atoms, and being optionally substituted with hydroxy, carboxy, C₁-C₆-alkyl or C₁-C₆-alkoxycarbonyl.
(vi) 5- to 6-membered heteroaryl comprising 2 to 4 heteroatoms selected from nitrogen atom, oxygen atom and sulfur atom, said heteroaryl being optionally substituted with one or more substituents selected from C₁-C₆-alkyl; amino; carboxy; C₁-C₆-alkoxy; C₁-C₆-alkoxycarbonyl; and C₆-C₁₀-aryl where the substituent is unsubstituted or mono- or disubstituted with hydroxy, cyano, carboxy, C₁-C₆-alkoxy, C₁-C₆-alkoxycarbonyl, C₃-C₆-cycloalkyl or C₆-C₁₀-aryl.
(vii) saturated or partially unsaturated, single or fused 3- to 10-membered heterocycle comprising 1 to 4 heteroatoms selected from nitrogen atom, oxygen atom and sulfur atom, said heterocycle being connected to the backbone through a ring member nitrogen and being optionally substituted with one or more substituents selected from the following groups:
(a) hydroxy, oxo, cyano, carboxy, hydroxyimino,
(b) amino being unsubstituted or mono- or disubstituted with C₁-C₆-alkyl (said alkyl is optionally substituted with hydroxy) or C₁-C₆-alkoxycarbonyl,
(c) carbamoyl being unsubstituted or mono- or disubstituted with C₁-C₆-alkyl, hydroxy, hydroxy-C₁-C₆-alkyl, amino-C₁-C₆-alkyl or C₆-C₁₀-aryl-C₁-C₆-alkylsulfonyl,
(d) C₁-C₆-alkoxyimino being optionally substituted with C₆-C₁₀-aryl,
(e) C₁-C₆-alkyl being optionally substituted with hydroxy, halogen or amino,
(f) C₁-C₆-alkoxy,
(g) C₁-C₆-alkylcarbonyl being optionally substituted with hydroxy or halogen,
(h) C₁-C₆-alkoxycarbonyl being optionally substituted with C₁-C₆-alkylcarbonyloxy,
(i) C₁-C₆-alkylsulfonyl,
(j) C₁-C₆-alkylcarbonyloxy,
(k) C₁-C₆-alkylcarbonylamino being optionally substituted with hydroxy; amino; cyano; halogen; C₁-C₆-alkoxy; or 5- to 6-membered heteroaryl comprising 1 to 2 heteroatoms selected from nitrogen atom and sulfur atom, and being optionally substituted with amino,
(l) C₃-C₆-cycloalkylcarbonylamino,
(m) 5- to 6-membered heteroarylcarbonylamino comprising 1 to 2 heteroatoms selected from nitrogen atom and oxygen atom in the heteroaryl,
(n) C₁-C₆-alkylsulfonylamino,
(o) C₆-C₁₀-aryl being optionally substituted with hydroxy,
(p) C₃-C₆-cycloalkyl, and
(q) C₃-C₆-cycloalkyl-C₁-C₆-alkyl.

3. The compound having the formula 1 according to claim 1, or pharmaceutically acceptable salt thereof, wherein:
T represents N or C,
P represents C₁-C₄-alkyl being optionally substituted with fluorine,
The substituent 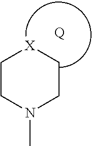
is optionally substituted with 1 to 2 substituents selected from the group consisting of oxo; C₁-C₄-alkyl being optionally substituted with fluorine; hydroxy-C₁-C₄-alkyl; C₁-C₄-alkoxy; phenyl; and furyl, and represents a heterocycle selected from the following structures: 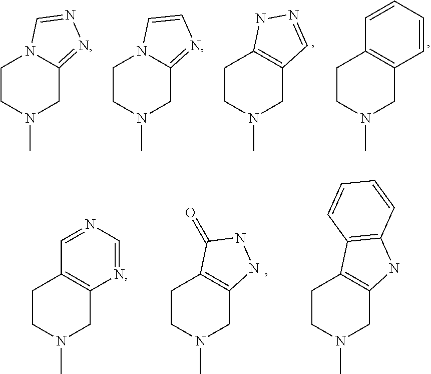
and
R represents a group selected from the following groups:
(i) —C₁-C₄-alkyl-R¹
wherein R¹ is selected from hydroxy; carboxy; carbamoyl; thiocarbamoyl; C₁-C₄-alkoxycarbonyl; phenyloxy being optionally substituted with carboxy or C₁-C₄-alkoxycarbonyl; benzoyloxy; thiazolyl comprising 1 to 2 heteroatoms selected from nitrogen atom and sulfur atom, and being optionally substituted with carboxy or C₁-C₄-alkoxycarbonyl; and pyrrolidinyl being optionally substituted with hydroxy.
(ii) —NR²R³
wherein each of R² and R³ is independently selected from hydrogen; C₁-C₄-alkyl being optionally substituted with amino (said amino is optionally substituted with carbamoyl), hydroxy, carboxy, hydroxyphenyl, C₁-C₄-alkoxy, C₁-C₄-alkoxycarbonyl, hydroxy-C₁-C₄-alkoxy, or pyrrolidinyl or thiazolidinyl being optionally substituted with oxo or benzyl; C₃-C₆-cycloalkyl being optionally substituted with hydroxy or hydroxy-C₁-C₄-alkoxy; 4- to 5-membered heterocycle comprising 1 nitrogen atom; pyrazolyl; phenyl; benzyl; pyrimidinyl; and thiazolyl being optionally substituted with carboxy or C₁-C₄-alkoxycarbonyl.
(iii) —O—R⁴
wherein R⁴ is selected from the following groups:
(a) hydrogen,
(b) C₁-C₄-alkyl being optionally substituted with hydroxy; C₁-C₄-alkoxy; amino (said amino is optionally substituted with formyl or C₁-C₄-alkylcarbonyl); oxo; C₁-C₄-alkylcarbonyloxy-C₁-C₄-alkoxy; phenyl being optionally substituted with halogen; pyridyl; oxazolyl being optionally substituted with carboxy-C₁-C₄-alkyl or C₁-C₄-alkoxycarbonyl-C₁-C₄-alkyl; 5-membered heterocycle comprising 1 heteroatom selected from nitrogen atom and oxygen atom, and being optionally substituted with oxo; or pyridylcarbonylamino,
(c) C₅-C₆-cycloalkyl being optionally benzo-fused,
(d) C₁-C₄-alkylamino-C₁-C₄-alkyl being optionally substituted with C₁-C₄-alkoxycarbonyl or carboxy,
(e) C₅-C₆-cycloalkylcarbonylamino-C₁-C₄-alkyl,
(f) C₅-C₆-cycloalkylsulfonylamino-C₁-C₄-alkyl,
(g) C₁-C₄-alkylcarbonylamino-C₁-C₄-alkyl being optionally substituted with hydroxy, halogen, amino, C₁-C₄-alkoxy, C₁-C₄-alkylsulfonyl or aminosulfonyl,
(h) C₁-C₄-alkylsulfonylamino-C₁-C₄-alkyl being optionally substituted with halogen,
(i) phenyl being optionally substituted with cyano; formyl; carboxy; C₁-C₄-alkoxycarbonyl; hydroxy-C₁-C₄-alkyl; carboxy-C₁-C₄-alkyl; C₁-C₄-alkoxycarbonyl-C₁-C₄-alkyl; carboxy-C₁-C₄-alkoxy; C₁-C₄-alkoxycarbonyl-C₁-C₄-alkoxy; or piperazinyl,
(j) tetrahydrofuryl; pyrrolidinyl being optionally substituted with C₁-C₄-alkyl; or acetidinyl,
(k) pyridyl.
(iv) —S—R⁵
wherein R⁵ is selected from phenyl, benzyl and pyrimidinyl.
(v) —C(═O)—R⁶
wherein R⁶ is selected from hydroxy; C₁-C₄-alkoxy; amino; C₁-C₄-alkylamino being optionally substituted with cyano, hydroxy, carboxy, C₁-C₄-alkoxycarbonyl or phenyl; phenylamino; and pyrrolidinyl, piperidinyl and piperazinyl being optionally substituted with hydroxy, carboxy, C₁-C₄-alkyl or C₁-C₄-alkoxycarbonyl.
(vi) oxadiazolyl, isoxadiazolyl, tetrazolyl, thiazolyl or pyrazolyl being optionally substituted with one or more substituents selected from C₁-C₄-alkyl; amino; carboxy; C₁-C₄-alkoxy; C₁-C₄-alkoxycarbonyl; and phenyl where the substituent is unsubstituted or mono- or disubstituted with hydroxy, cyano, carboxy, C₁-C₄-alkoxy, C₁-C₄-alkoxycarbonyl, C₃-C₆-cycloalkyl or phenyl.
(vii) heterocycle selected from the following structures and optionally substituted with one or more substituents selected from the groups (a) to (q): 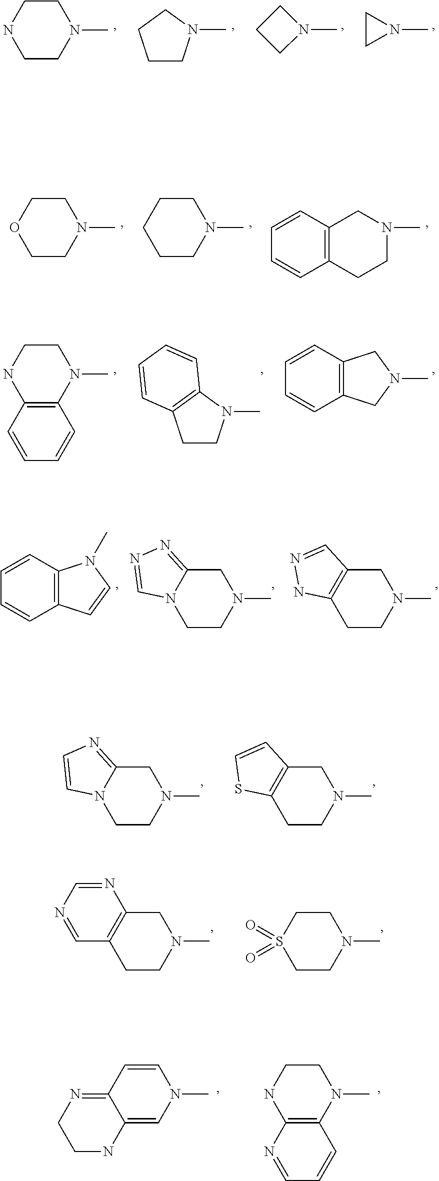
(a) hydroxy, oxo, cyano, carboxy, hydroxyimino,
(b) amino being unsubstituted or mono- or disubstituted with C₁-C₄-alkyl (said alkyl is optionally substituted with hydroxy) or C₁-C₄-alkoxycarbonyl,
(c) carbamoyl being unsubstituted or mono- or disubstituted with C₁-C₄-alkyl, hydroxy, hydroxy-C₁-C₄-alkyl, amino-C₁-C₄-alkyl or benzylsulfonyl,
(d) C₁-C₄-alkoxyimino being optionally substituted with phenyl,
(e) C₁-C₄-alkyl being optionally substituted with hydroxy, halogen or amino,
(f) C₁-C₄-alkoxy,
(g) C₁-C₄-alkylcarbonyl being optionally substituted with hydroxy or halogen,
(h) C₁-C₄-alkoxycarbonyl being optionally substituted with C₁-C₄-alkylcarbonyloxy,
(i) C₁-C₄-alkylsulfonyl,
(j) C₁-C₄-alkylcarbonyloxy,
(k) C₁-C₆-alkylcarbonylamino being optionally substituted with hydroxy; amino; cyano; halogen; C₁-C₄-alkoxy; or thiazolyl, imidazolyl or pyridyl being optionally substituted with amino,
(l) C₃-C₆-cycloalkylcarbonylamino,
(m) pyridylcarbonylamino or furylcarbonylamino,
(n) C₁-C₄-alkylsulfonylamino,
(o) phenyl being optionally substituted with hydroxy,
(p) C₃-C₆-cycloalkyl, and
(q) C₃-C₆-cycloalkyl-C₁-C₄-alkyl.

4. A pharmaceutical composition for preventing and treating vascular disease, comprising the compound having the formula 1 according to claim 1 or pharmaceutically acceptable salt thereof as active ingredient, and pharmaceutically acceptable carrier.

5. The pharmaceutical composition according to claim 4 wherein the composition is used for inhibition of circulatory disease related to thrombus formation resulting from platelet aggregation; acceleration of platelet separation; antithrombotic; reconstructive surgery including skins and muscle flap; or mechanically induced platelet activation in the organism.

6. The pharmaceutical composition according to claim 4 wherein the vascular disease is selected from stable or unstable angina pectoris, primary arterial thrombotic complication of atherosclerosis, transient ischemic attack, peripheral vascular disease, myocardial infarction with or without thrombolytic agent, arterial complication resulting from the involvement of atherosclerotic disease, thrombotic complication of surgery or mechanical injury, disseminated intravascular coagulation, thrombotic thrombocytopenic purpura, hemolytic uremic syndrome, thrombotic complication of sepsis, adult respiratory distress syndrome, heparin induced thrombocytopenia, preeclampsia, eclampsia, deep venous thrombosis, intravenous thrombosis, thrombocytopenia, myeloproliferative disorder, drepanocytemia, shunt occlusion, vasculitis, arteritis, glomerulonephritis, secondary thrombosis of vascular injury or inflammation, hemicranicus, Raynaud's phenomenon, platelet atheroma plague formation and progression, coarctation and restenosis, and inflammation, asthma, central nervous disease, or tumor growth and extension associated with platelet and platelet-induced factors.

7. The pharmaceutical composition according to claim 4 wherein the vascular disease is selected from phlebothrombosis, thrombophlebitis, arterial embolism, coronary artery and cerebral artery thrombosis, myocardial infarction, stroke, cerebral embolism, kidney embolism, pulmonary embolism, thrombotic apoplexy, transient ischemic attack, peripheral vascular disease and stable and unstable angina pectoris.
